(19) United States
(12) Patent Application Publication
(10) Pub. No.: US 2006/0287337 A1
(43) Pub. Date: Dec. 21, 2006

(54) Trans-golgi network-associated processes, methods and compositions related thereto

(75) Inventors: Yuval Reiss, Kiriat-Ono (IL); Iris Alroy, Ness-Ziona (IL); Shmuel Tuvia, Netanya (IL); Liora Yaar, Raanana (IL); Daniel N. Taglicht, Lapid (IL)

Correspondence Address: FISH & NEAVE IP GROUP;ROPES & GRAY LLP, ONE INTERNATIONAL PLACE, BOSTON, MA, 02110-2624, US

(73) Assignee: Proteologics, Inc., Orangeburg, NY (US)

(21) Appl. No.: 11/127,559

(22) Filed: May 11, 2005

Related U.S. Application Data
(63) Continuation of application No. PCT/US03/35712, filed on Nov. 10, 2003.
Application No. PCT/US03/35712 is a continuation-in-part of application No. 10/293,965, filed on Nov. 12, 2002.
(60) Provisional application No. 60/345,846, filed on Nov. 9, 2001.
(60) Provisional application No. 60/364,530, filed on Mar. 15, 2002.
(60) Provisional application No. 60/443,495, filed on Jan. 29, 2003.
(60) Provisional application No. 60/475,387, filed on Jun. 2, 2003.
(60) Provisional application No. 60/491,891, filed on Aug. 1, 2003.

(30) Foreign Application Priority Data
Nov. 11, 2002 (EP) 02257796.9
Nov. 12, 2002 (WO) PCT/US02/36366

(51) Int. Cl. A61K 31/513; A61K 31/496; A61K 31/4166; A61K 31/44; A61K 31/4015; A61K 31/13

(52) U.S. Cl. 514/255.01; 514/270; 514/352; 514/389; 514/425; 514/641

(57) ABSTRACT

The application discloses methods and compositions related to the modulation of trans-Golgi network-associated processes, including methods and compositions for the modulation of trans-Golgi network-associated proteins and methods for the treatment of disorders associated with trans-Golgi network processes.

### RELATED APPLICATIONS

This application is a continuation of International Patent Application PCT/US03/35712, filed on Nov. 10, 2003, designating the U.S., which claims the benefit of the filing date of European Patent Application No. 02257796.9, filed Nov. 11, 2002, U.S. patent application Ser. No. 10/293,965, filed Nov. 12, 2002, PCT International Application US02/36366, filed Nov. 12, 2002, and U.S. Provisional Application Nos. 60/443,495, filed Jan. 29, 2003, 60/475,387, filed Jun. 2, 2003, 60/491,891, filed Aug. 1, 2003, the specifications of which are hereby incorporated by reference in their entirety. International Application PCT/US03/35712 was published under PCT Article 21(2) in English.

### BACKGROUND

Potential drug target validation involves determining whether a DNA, RNA or protein molecule is implicated in a disease process and is therefore a suitable target for development of new therapeutic drugs. Drug discovery, the process by which bioactive compounds are identified and characterized, is a critical step in the development of new treatments for human diseases. The landscape of drug discovery has changed dramatically due to the genomics revolution. DNA and protein sequences are yielding a host of new drug targets and an enormous amount of associated information.

The identification of genes and proteins involved in various disease states or key biological processes, such as inflammation and immune response, is a vital part of the drug design process. Many diseases and disorders could be treated or prevented by decreasing the expression of one or more genes involved in the molecular etiology of the condition if the appropriate molecular target could be identified and appropriate antagonists developed. For example, cancer, in which one or more cellular oncogenes become activated and result in the unchecked progression of cell cycle processes, could be treated by antagonizing appropriate cell cycle control genes. Furthermore many human genetic diseases, such as Huntington's disease, and certain prion conditions, which are influenced by both genetic and epigenetic factors, result from the inappropriate activity of a polypeptide as opposed to the complete loss of its function. Accordingly, antagonizing the aberrant function of such mutant genes would provide a means of treatment. Additionally, infectious diseases such as HIV have been successfully treated with molecular antagonists targeted to specific essential retroviral proteins such as HIV protease or reverse transcriptase. Drug therapy strategies for treating such diseases and disorders have frequently employed molecular antagonists which target the polypeptide product of the disease gene(s). However the discovery of relevant gene or protein targets is often difficult and time consuming.

The vesicular trafficking systems are the major pathways for the distribution of proteins among cell organelles, the plasma membrane and the extracellular medium. A central vesicular trafficking system involves the manipulation and passage of nascent proteins from the endoplasmic reticulum to the Golgi complex as well as the trafficking of protein complexes, including protein-nucleic acid complexes (e.g., protein-RNA, protein-DNA). The Golgi complex represents a major processing and sorting compartment for proteins destined for secretion or delivery to the cell surface or to lysosomes. A key component of this vesicular trafficking pathway is the trans-Golgi network (“TGN”). The Golgi complex is made up of multiple membrane-bound, flattened cisternae, and the TGN comprises the most distal of these. The individual cisternae or pairs of adjacent cisternae of the Golgi complex contain distinct sets of proteins. For example, the oligosaccharide-modification enzyme, galactosyltransferase, is selectively found within the trans-Golgi compartments. Additionally, the lipid composition changes from one side of the Golgi stack of cisternae to the other.

The TGN is a tubular-reticular structure that is thought to be responsible for sorting secretory proteins to their final destinations. Distinct membrane vesicles, both clathrin-coated vesicles and vesicles comprising a “lace-like” coat, form and bud from specific tubules of the TGN. Involved in the formation of these coats at the TGN are GGA proteins (for Golgi-localized, gamma-ear containing, ADP-ribosylation factor-binding proteins). GGA proteins are involved in trafficking of proteins from the TGN to endosomes. They are involved in protein cargo selection, membrane localization, binding to clathrin, and accessory protein recruitment. GGA proteins are recruited to the TGN by a member of the ARF family of small GTPases. ARF is a protein that is required for clathrin-coated vesicle formation, but not for the formation of lacy-coated vesicles of the TGN.

The major vesicle trafficking systems in eukaryotic cells include those systems that are mediated by clathrin-coated vesicles and coatomer-coated vesicles. Clathrin-coated vesicles are generally involved in transport, such as from the TGN to endosomes as well as in the case of receptor mediated endocytosis, between the plasma membrane and the early endosomes. Coatomer-coated vesicles include coat protein I (COP-I) coated vesicles and COP-II coated vesicles, both of which tend to mediate transport of a variety of molecules between the ER and Golgi cisternae. In each case, a vesicle is formed by budding out from a portion of membrane that is coated with coat proteins, and the vesicle sheds its coat prior to fusing with the target membrane.

Clathrin coats assemble on the cytoplasmic face of a membrane, forming pits that ultimately pinch off to become vesicles. Clathrin itself is composed of two subunits, the clathrin heavy chain and the clathrin light chain, that form the clathrin triskelion. Clathrins associate with a host of other proteins, including the assembly protein, AP180, the adaptor complexes (AP1, AP2, AP3 and AP4), beta-arrestin, arrestin 3, auxilin, epsin, Eps15, v-SNAREs, amphiphysins, dynamin, synaptojanin and endophilin. The adaptor complexes promote clathrin cage formation, and help connect clathrin up to the membrane, membrane proteins, and many of the preceding components. AP1 associates with clathrin coated vesicles derived from the trans-Golgi network and contains γ, β1, μ1 and σ1 polypeptide chains. AP2 associates with endocytic clathrin coated vesicles and contains α, β2, σ2, and σ2 polypeptides. Interactions between the clathrin complex and other proteins are mediated by a variety of domains found in the complex proteins, such as SH3 (Src homology 3) domains, PH (pleckstrin homology) domains, EH domains and NPF domains. (Marsh et al. (1999) Science 285:215-20; Pearse et al. (2000) Curr Opin Struct Biol 10(2):220-8).

Coatomer-coated vesicle formation is initiated by recruitment of a small GTPase (e.g., ARF or SAR) by its cognate guanine nucleotide exchange factor (e.g., SEC12, GEA1, GEA2). The initial complex is recognized by a coat protein complex (COPI or COPII). The coat then grows across the membrane, and various cargo proteins become entrapped in the growing network. The membrane ultimately bulges and becomes a vesicle. The coat proteins stimulate the GTPase activity of the GTPase, and upon hydrolysis of the GTP, the coat proteins are released from the complex, uncoating the vesicle. Other proteins associated with coatomer coated vesicles include v-SNAREs, Rab GTPases and various receptors that help recruit the appropriate cargo proteins. (Springer et al. (1999) Cell 97:145-48).

The vesicular environment of the TGN contributes to the presence of subdomains within the TGN compartments. Clathrin-coated vesicles form specifically from the last cisternal stack of the TGN, and clathrin-coated microdomain formation in the TGN may involve the action of TGN-resident guanine nucleotide exchange factors requisite for ARF recruitment to the TGN. These exchange factors are varied and demonstrate specificity for different ARF subtypes. They are localized uniquely within the TGN, and the different localizations of each type of exchange factor may represent specific subdomain formations within the TGN. Protein kinase D represents another protein, the presence of which is associated with what may be classified as a subdomain of the TGN. Protein kinase D is involved in the formation of vesicles from the TGN that comprise lace-like coats, and is involved in the regulated transport of vesicles from the TGN to the plasma membrane.

In addition to the involvement of proteins in the manipulation and vesicular transport of proteins from the TGN, the role of lipids is also important in TGN function and composition. The secretory pathway involves various forms of phosphoinositides and the hydrolyzed products from phosphatidylcholine, such as phosphatidic acid. The production and balance of these lipids are controlled by various lipid kinases and lipid transfer proteins, which tightly regulate membrane transport, including membrane transport to and from the TGN.

Manipulation and transport of proteins by the TGN involves proteins targeted to the TGN. The polypeptide ubiquitin is involved in certain of these TGN trafficking events. For instance, the transport of amino acid permeases, which are involved in the transport of amino acids into cells from the extracellular environment, is mediated by the TGN. The transport of amino acid permeases to either the plasma membrane or to the lysosome is determined in the TGN, and ubiquitination of these permeases has been implicated in their targeted transport from the TGN to the lysosome for degradation.

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, DNA repair, and intracellular transport. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and generally also requires auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin may then be transferred to a specific cysteine on one of several E2 enzymes. Finally, these E2 enzymes donate ubiquitin to protein substrates, typically with the assistance of a C3 protein, also known as a ubiquitin enzyme. In certain instances, substrates are recognized directly by the ubiquitin-conjugated E2 enzyme.

It is also known that the ubiquitin system plays a role in a wide range of cellular processes including cell cycle progression, apoptosis, and turnover of many membrane receptors. Additionally, ubiquitin has been tied to neuropathologic conditions. In viral infections, the ubiquitin system is involved not only with assembly, budding and release, but also with repression of host proteins such as p53, which may lead to a viral-induced neoplasm. The HIV Vpu protein interacts with an E3 protein that regulates IκB degradation, and is thought to promote apoptosis of infected cells by indirectly inhibiting NF-κB activity (Bour et al. (2001) J Exp Med 194:1299-311; U.S. Pat. No. 5,932,425). The ubiquitin system regulates protein function by both mono-ubiquitination and poly-ubiquitination, and poly-ubiquitination is primarily associated with protein degradation.

One area of particular interest is the identification of host genes and proteins that are co-opted by viruses during the viral life cycle. The serious and incurable nature of many viral diseases, coupled with the high rate of mutations found in many viruses, makes the identification of antiviral agents a high priority for the improvement of world health. Genes and proteins involved in a viral life cycle are also appealing as a subject for investigation because such genes and proteins will typically have additional activities in the host cell and may play a role in other non-viral disease states.

Viral maturation involves the proteolytic processing of the Gag proteins, organization of viral proteins and RNA to form a ribonucleoparticle, and the activity of various host proteins. It is believed that cellular machineries for exo/endocytosis and for ubiquitin conjugation may be involved in the maturation. In particular, the assembly, budding and subsequent release of retroid viruses, RNA viruses and envelop viruses, such as various retroviruses, rhabdoviruses, lentiviruses, and filoviruses may involve the Gag polyprotein. After its synthesis, Gag is targeted to the plasma membrane where it induces budding of nascent virus particles.

The role of ubiquitin in virus assembly was suggested by Dunigan et al. (1988, Virology 165, 310, Meyers et al. 1991, Virology 180, 602), who observed that mature virus particles were enriched in unconjugated ubiquitin. More recently, it was shown that proteasome inhibitors suppress the release of HIV-1, HIV-2 and virus-like particles derived from SIV and RSV Gag. Also, inhibitors affect Gag processing and maturation into infectious particles (Schubert et al 2000, PNAS 97, 13057, Harty et al. 2000, PNAS 97, 13871, Strack et al. 2000, PNAS 97, 13063, Patnaik et al. 2000, PNAS 97, 13069).

The vesicular trafficking systems may be directly or indirectly involved in a variety of disease states. Many degenerative neurological pathologies, for instance, involve the ectopic presence or accumulation of proteins transited through vesicular trafficking systems. Likewise, a number of cancers involve proteins that are transported intracellularly and secreted into the extracellular environment.

It would be beneficial to identify proteins involved in one or more of these processes for use in, among other things, drug screening methods.

### SUMMARY

In certain embodiments, the present invention relates to inhibiting a process associated with the trans-Golgi network (TGN) by modulating a TGN-associated polypeptide. A TGN-associated polypeptide may be one or more of a POSH polypeptide, a POSH-pathway polypeptide (a polypeptide involved in a signaling pathway associated with a POSH polypeptide), or a POSH-associated polypeptide (“POSH-AP”). POSH may participate in biological processes independently of any or all POSH-APs. Likewise, POSH-APs, though associated with POSH in certain biological contexts, may participate in biological processes independently of POSH.

TGN-associated processes include the processing of proteins. Processing of these proteins includes post-translational modification of the protein, such as, e.g., gylcosylation, sulfation, cleavage of one or more amino acids, and assembly of the protein into a protein complex. TGN-associated processes also include the localization and trafficking of proteins through the TGN, including the trafficking of proteins from the endoplasmic reticulum (ER) to the plasma membrane or to lysosomes, both enterograde and retrograde. Proteins processed by the TGN include viral, bacterial, parasitic, and other microbial proteins. TGN-associated processes also include the processing of protein complexes, including protein complexes comprising nucleic acid, such as protein-RNA or protein-DNA complexes. TGN-associated processes further include the assembly and/or targeting of ribonucleoparticles. In certain aspects, the application relates to modulation of a TGN-associated protein complex. In certain embodiments, the TGN-associated protein complex comprises at least one TGN-associated protein. In further embodiments, the TGN-associated protein complex further comprises nucleic acid.

TGN-associated processes also encompass processing, including aberrant processing, of proteins that are associated with neurological disorders. Aberrant processing of proteins associated with neurological disorders includes increased or decreased amounts of the protein in the TGN. Aberrant processing also includes incomplete processing of the protein as well as improper glycosylation of the protein. Proteins aberrantly processed by the TGN that are associated with one or more neurological disorders are encompassed in methods of the invention. Neurological disorders include Alzheimer's disease, Parkinson's disease, Huntington's disease, schizophrenia, and prion-associated diseases.

TGN-associated processes further include the processing, including the aberrant processing, of proteins that are associated with neoplastic conditions. Proteins incorrectly processed include proteins associated with neoplastic conditions such as cancers, which include thyroid carcinoma, liver cancer, lung cancer, cervical cancer, colorectal cancer, ovarian cancer, renal cell carcinoma, lymphoma, osteosarcoma, prostate cancer, liposarcoma, leukemia, breast carcinoma, and breast adeno-carcinoma. Aberrant processing of the protein includes mislocalization of the protein. Aberrant processing also encompasses changes, such as an increase or a decrease, in the transport of the protein from the TGN to the plasma membrane when compared to the level of transport of the protein under physiological conditions. Aberrant processing further includes increased or decreased secretion of the protein.

TGN-associated processes additionally include aberrant processing of proteins associated with immunological disorders. Immunological disorders include inflammatory diseases and autoimmune diseases. TGN-associated processes further include aberrant processing of proteins associated with cardiovascular disorders.

In certain aspects, the application relates to inhibiting the secretion of proteins. In one embodiment, the application relates to regulating the intracellular transport of myristoylated proteins from the TGN. Exemplary myristoylated proteins include Gag, Src, HIV-1 Nef, and Rapsyn. In one embodiment, the application relates to a method of inhibiting the secretion of proteins comprising modulating one or more of a TGN-associated polypeptide. Exemplary TGN-associated polypeptides include the POSH-APs HERPUD1, GOCAP1, GOSR2, PKA and Cbl-b. An exemplary TGN-associated polypeptide that is a POSH-pathway polypeptide is phospholipase D (“PLD”).

In additional embodiments of the present application, the application relates to methods of modulating the activity of a TGN-associated protein. An embodiment of the application relates to a method of modulating the activity of a TGN-associated protein comprising modulating the activity of one or more of a POSH, POSH-pathway, and/or a POSH-AP polypeptide.

In certain aspects, the application relates to TGN-associated proteins that comprise a TGN-localization domain. A TGN-localization domain localizes a protein to the TGN and/or is associated with the protein's retention in the TGN. TGN-localization domains include dileucine motifs; the tyrosine-based motif, YXXO, wherein the O is an amino acid comprising a bulky, hydrophobic group; an acidic amino acid cluster; a casein kinase II phosphorylation site; a VHS domain; a GAT domain; an ear domain; a GRIP domain; an ENTH domain; a cysteine rich domain; and/or a granin motif.

In additional embodiments, the application relates to methods of screening for agents possessing anti-viral activity. In one embodiment, the application relates to a method of screening for an anti-viral agent comprising (a) contacting a cell with a test agent; and (b) evaluating assembly or transport of a viral protein or nucleic acid (e.g., RNA) in the TGN in the presence of the agent; and (c) comparing the assembly or transport of the viral protein in the absence of the agent, wherein inhibition or disruption of the assembly or transport is indicative of an agent having anti-viral activity. In particularly preferred embodiments, a test agent will have a lesser effect on the trafficking of host cell proteins.

The application further relates to a method of screening for an agent that is capable of inhibiting the progression of a neurological disorder. In certain embodiments, the method comprises (a) contacting a cell with a test agent; and (b) evaluating aberrant processing of a protein in the TGN in the presence of the agent; and (c) comparing the processing of the protein in the absence of the agent, wherein inhibition or disruption of the aberrant processing is indicative of an agent capable of inhibiting the progression of a neurological disorder.

In certain aspects, the application relates to a method of screening for an agent possessing anti-proliferative or anti-apoptotic activity. In certain embodiments, the method comprises (a) contacting a cell with a test agent; and (b) evaluating aberrant processing of a protein in the TGN in the presence of the agent; and (c) comparing the processing of the protein in the absence of the agent, wherein inhibition or disruption of the aberrant processing is indicative of an agent possessing anti-proliferative or anti-apoptotic activity.

In certain aspects the application provides methods for inhibiting the maturation of a virus by affecting the assembly and/or movement of viral proteins in the secretory pathway of an infected cell.

In certain embodiments, the application provides methods for inhibiting maturation or abrogating infectivity of a virus, such as a lentivirus (e.g., HIV) or a flavivirus (e.g., West Nile virus), comprising inhibiting the transport of a viral protein or nucleic acid, such as RNA, assembly from the trans Golgi network to the plasma membrane. In certain embodiments, the application provides methods for inhibiting the maturation or abrogating infectivity of a virus, such as a lentivirus (e.g., HIV) or a flavivirus (e.g., West Nile virus), the method comprising disrupting the formation of a viral protein assembly in the trans Golgi network. In certain embodiments, the transport of a viral protein assembly from the trans Golgi network to the plasma membrane may be inhibitied by modulating an activity of a POSH polypeptide, a POSH-pathway polypeptide or POSH-AP.

In certain embodiments, the application provides methods for selecting host cell polypeptide targets for antiviral therapy by selecting host cell polypeptides that are localized, or known to be localized, to the trans Golgi (though the polypeptide need not be exclusively localized in the trans Golgi). A method may further comprise impairing the function of the selected polypeptide and evaluating the effect on maturation of a virus or virus-like particle, such as an HIV virus or virus-like particle. In certain embodiments, a decrease in the function of a good target for antiviral therapy causes the accumulation of intracellular viral aggregates (e.g., virus-like particles). Optionally, a decrease in the function of the test polypeptide is achieved by decreasing expression through a knockdown by, e.g., RNAi, or by knockout (conditional or complete) of the endogenous gene.

In some aspects, the invention provides TGN-associated nucleic acid sequences and proteins encoded thereby, as well as oligonucleotides derived from the nucleic acid sequences, antibodies directed to the encoded proteins, screening assays to identify agents that modulate TGN-associated polypeptides, and diagnostic methods for detecting cells infected with a virus, preferably an envelop virus, an RNA virus and particularly a retroidvirus.

In an additional aspect, the invention provides nucleic acid therapies for manipulating TGN-associated proteins, such as POSH, POSH-pathway, or POSH-AP polypeptides. In one embodiment, the invention provides a ribonucleic acid comprising between 5 and 1000 consecutive nucleotides of a nucleic acid sequence that is at least 90%, 95%, 98%, 99% or optionally 100% identical to a sequence of SEQ ID NO:1 and/or 3 or a complement thereof. Optionally the ribonucleic acid comprises at least 10, 15, 20, 25, or 30 consecutive nucleotides, and no more than 1000, 750, 500 and 250 consecutive nucleotides of a POSH nucleic acid. In certain embodiments the ribonucleic acid is an RNAi oligomer or a ribozyme. Preferably, the ribonucleic acid decreases the level of a TGN-associated polypeptide mRNA. Preferred ribonucleic acids comprise a sequence selected from any of SEQ ID NOs: 15, 16, 18, 19,21, 22, 24 and 25.

In certain embodiments, the application provides methods employing a ribonucleic acid comprising between 5 and 1000 consecutive nucleotides of a nucleic acid sequence that is at least 90%, 95%, 98%, 99% or optionally 100% identical to a sequence of SEQ ID NOS: 37-74, 98-115 or a complement thereof.

The invention also features transgenic non-human animals, e.g., mice, rats, rabbits, goats, sheep, dogs, cats, cows, or non-human primates, having a transgene, e.g., animals which include (and preferably express) a heterologous form a TGN-associated polypeptide gene. Such a transgenic animal can serve as an animal model for studying viral infections such as HIV infection or for use in drug screening for viral infections. Such a transgenic animal can serve as an animal model for studying the progression of neurological disorders such as Alzheimer's disease or for use in drug screening for treatments for Alzheimer's disease.

In further aspects, the invention provides compositions for the delivery of a nucleic acid therapy, such as, for example, compositions comprising a liposome and/or a pharmaceutically acceptable excipient or carrier.

In further aspects, the application provides an isolated, purified or recombinant complex comprising a TGN-associated polypeptide. In certain aspects, the application provides methods for identifying a test agent having antiviral or anti-apoptotic activities by identifying a test agent that disrupts a complex described above. In certain aspects, the application provides methods for identifying a test agent having activity against the onset or progression of a neurological disorder by identifying a test agent that disrupts a complex described above.

In certain aspects the application provides an isolated antibody, or fragment thereof, specifically immunoreactive with an epitope of a sequence selected from the group consisting of SEQ ID NO: 2, SEQ ID NO: 26, and SEQ ID NO:30, which antibody disrupts the interaction of a TGN-associated polypeptide. In certain embodiments, the application provides an isolated antibody, or fragment thereof, specifically immunoreactive with an epitope of a sequence selected from the group consisting of SEQ ID NOS: 75-97 and 116-125, which antibody disrupts the interaction of a TGN-associated polypeptide.

In certain aspects, the application provides for the use of an agent that inhibits a TGN-associated protein of the host cell for making a medicament for inhibiting the processing of a viral, bacterial, parasitic or microbial protein. Optionally, the TGN-associated protein is selected from among: POSH, a POSH-pathway polypeptide, a POSH-AP and a POSH binding protein. Examples of such TGN-associated proteins are: Cbl-b, HERPUD1, GOCAP1, GOSR2, a PKA subunit, DDEF1, ARHV (CHP), SPG20, CENTB1, dynaminII, and RALA. In certain embodiments, processing of the viral protein comprises transport of the viral proteins from the TGN to the plasma membrane. Processing of the viral proteins may comprise formation of a viral protein assembly in the TGN. A virus may be, for example, a lentivirus or a flavivirus.

In certain aspects, the application provides for the use of an agent that modulates a TGN-associated polypeptide for making a medicament for inhibiting aberrant processing of a protein associated with a neurological disorder. Optionally, the TGN-associated polypeptide is selected from among: POSH, a POSH-pathway polypeptide, a POSH-AP and a POSH binding protein. In a preferred embodiment, the neurological disorder is Alzheimer's disease and the agent inhibits POSH or HERPUD1.

In certain aspects, the application provides for the use of an agent that inhibits a TGN-associated polypeptide for making a medicament for inhibiting the aberrant processing of a protein, the aberrant processing of which is associated with a proliferative disorder. Optionally, the TGN-associated protein is selected from among: POSH, a POSH pathway polypeptide, a POSH-AP and a POSH binding protein. In a preferred embodiment, the proliferative disorder is breast cancer or colorectal cancer.

In certain aspects, the application provides for the use of an agent that modulates a TGN-associated protein for making a medicament for inhibiting the aberrant processing of a protein, wherein the aberrantly processed protein is associated with an immunological disorder. Optionally, the TGN-associated protein is selected from among: POSH, a POSH-pathway polypeptide, a POSH-AP and a POSH binding protein. In certain embodiments, the immune disorder is selected from among: an inflammatory disease and an autoimmune disease.

In certain aspects, the application provides for the the use of an agent that inhibits a TGN-associated polypeptide for making a medicament for inhibiting the secretion of a protein. Optionally, the TGN-associated polypeptide is selected from among: POSH, a POSH-pathway polypeptide, a POSH-AP and a POSH binding protein. Examples of such polypeptides include: Cbl-b, HERPUD1, GOCAP1, GOSR2, a PKA subunit, DDEF1, ARHV (CHP), SPG20, CENTB1, dynaminII, and RALA. In certain embodiments, the medicament is for inhbiting the secretion of a viral protein and/or a myristoylated protein, such as Gag, Src, Rapsyn, or HIV-1 Nef.

In certain aspects, the invention provides agents that may be used in any of the various methods for affecting a TGN-related process. In certain embodiments, an agent is an RNAi construct, an antisense construct, an antibody or a small molecule. Optionally, an RNAi or antisense construct inhibits expression of a polypeptide selected from among the following: POSH, Cbl-b, HERPUD1, GOCAP1, GOSR2, a PKA subunit, DDEF1, ARHV (CHP), SPG20, CENTB1, dynaminII, and RALA. Optionally, a small molecule is employed, such as

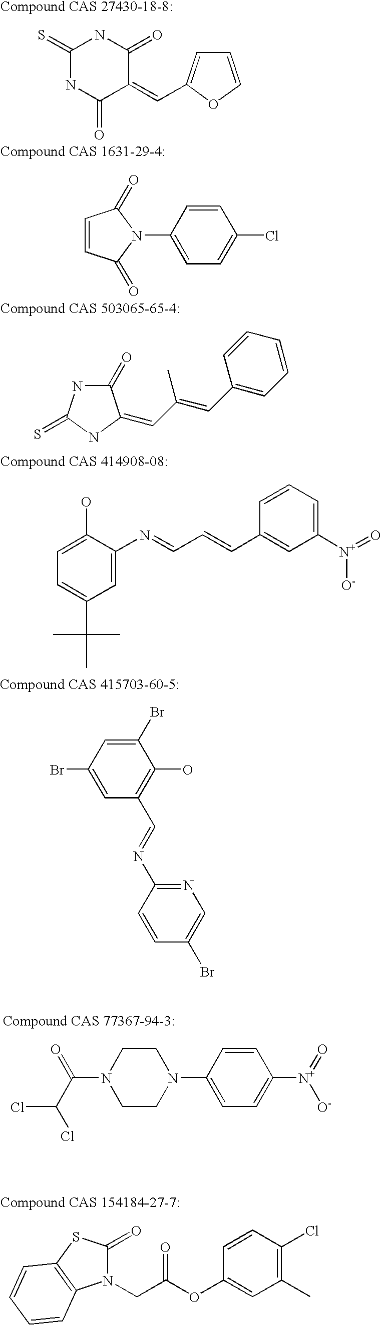

In certain embodiments, compounds useful in the instant compositions and methods include heteroarylmethylene-dihydro-2,4,6-pyrimidinetriones and their thione analogs. Preferred heteroaryl moieties include 5-membered rings such as thienyl, furyl, pyrrolyl, oxazolyl, thiazolyl, and imidazolyl moieties.

In certain embodiments, compounds useful in the instant compositions and methods include N-arylmaleimides, especially N-phenylmaleimides, in which the phenyl group may be substituted or unsubstituted.

In certain embodiments, compounds useful in the instant compositions and methods include arylallylidene-2,4-imidazolidinediones and their thione analogs. Preferred aryl groups are phenyl groups, and both the aryl and allylidene portions of the molecule may be substituted or unsubstituted.

In certain embodiments, compounds useful in the instant compositions and methods include substituted distyryl compounds and aza analogs thereof such as substituted 1,4-diphenylazabutadiene compounds.

In certain other embodiments, compounds useful in the instant compositions and methods include substituted styrenes and aza analogs thereof, such as 1,2-diphenylazaethylenes and 1-phenyl-2-pyridyl-azaethelenes.

In yet other embodiments, compounds useful in the instant compositions and methods include N-aryl-N′-acylpiperazines. In such compounds, the aryl ring, the acyl substituent, and/or the piperazine ring may be substituted or unsubstituted.

In additional embodiments, compounds useful in the instant compositions and methods include aryl esters of (2-oxo-benzooxazol-3-yl)-acetic acid, and analogs thereof in which one or more oxygen atoms are replaced by sulfur atoms.

Optionally, a small molecule inhibits the ubiquitin ligase activity of an E3 enzyme, such as POSH or Cbl-b.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1: Knockdown of POSH mRNA by siRNA duplexes. HeLa SS-6 cells were transfected with siRNA against Lamin A/C (lanes 1, 2) or POSH (lanes 3-10). POSH siRNA was directed against the coding region (153—lanes 3,4; 155—lanes 5,6) or the 3′UTR (157—lanes 7, 8; 159—lanes 9, 10). Cells were harvested 24 hours post-transfection, RNA extracted, and POSH mRNA levels compared by RT-PCR of a discrete sequence in the coding region of the POSH gene. GAPDH is used an RT-PCR control in each reaction.

FIG. 2: POSH affects the release of VLP from cells. A) Phosphohimages of SDS-PAGE gels of immunoprecipitations of ³⁵S pulse-chase labeled Gag proteins are presented for cell and viral lysates from transfected HeLa cells that were either untreated or treated with POSH RNAi (50 nM for 48 hours). The time during the chase period (1,2,3,4 and 5 hours after the pulse) are presented from left to right for each image.

FIG. 3: Release of VLP from cells at steady state. Hela cells were transfected with an HIV-encoding plasmid and siRNA. Lanes 1, 3 and 4 were transfected with wild-type HIV-encoding plasmid. Lane 2 was transfected with an HIV-encoding plasmids which contains a point mutation in p6 (PTAP to ATAP). Control siRNA (lamin A/C) was transfected to cells in lanes 1 and 2. siRNA to Tsg101 was transfected in lane 4 and siRNA to POSH in lane 3.

FIG. 4: Partial knockdown of human POSH results in four logs reduction of HIV1 infectivity. The results from infectivity assay are presented are presented in the diagram. The vertical axis shows the percentage of target cells infected, and the horizontal axis shows the fold dilution of virus stocks used (see Example 4 for details of the experiment). The light squares (top line) indicate the results from the control, and the dark squares (bottom line) indicate the results from transfecting cells with RNAi to POSH.

FIG. 5: Human POSH has ubiquitin ligase activity

FIG. 6: Human POSH co-immunoprecipitates with RAC1

FIG. 7: Knock-down of human POSH entraps HIV virus particles in intracellular vesicles. HIV virus release was analyzed by electron microscopy following siRNA and full-length HIV plasmid transfection. Mature viruses were secreted by cells transfected with HIV plasmid and non-relevant siRNA (control, bottom panel). Knockdown of Tsg101 protein resulted in a budding defect, the viruses that were released had an immature phenotype (top panel). Knockdown of hPOSH levels resulted in accumulation of viruses inside the cell in intracellular vesicles (middle panel).

FIG. 8 shows micrographs obtained by fluorescence microscopy of Hela SS6 cells that were either untreated (control), treated with Brefeldin A (BFA), subjected to siRNA depletion of POSH (POSH siRNA) or infected with an HIV construct. Cells were stained with anti-POSH and anti-phospholipase D (PLD) antibodies. PLD is a protein that is secreted through the trans Golgi network.

FIG. 9 shows micrographs obtained by fluorescence microscopy of Hela SS6 cells that were either untreated (control), treated with Brefeldin A (BFA), subjected to siRNA depletion of POSH (POSH siRNA) or infected with an HIV construct. Cells were stained with anti-POSH and anti-GM130 antibodies. GM130 is a marker for the cis-Golgi network.

FIG. 10. POSH knockdown results in decreased secretion of phospholipase D (“PLD”).

FIG. 11. POSH knockdown decreases the release of extracellular MMuLV particles.

FIG. 12. POSH is localized to the trans-Golgi (TGN). HeLa SS6 cells were incubated for 1 h at 40° C. to block vesicular transport. Cells were than fixed in 3.7% paraformaldehyde and permeablized. Cells were co-stained with sheep anti-TGN46 antibody (green) and with rabbit anti-POSH antibody (red).

FIGS. 13A-B. (A) Growth curve of HeLa SS cells as a function of time. Control siRNA (triangles), POSH siRNA (squares). (B) Levels of POSH expression as a function of time after POSH siRNA transfection.

FIGS. 14A-B. (A) Expression profile of POSH protein in different cell lines. (B) Tissue-specific expression of POSH protein.

FIG. 15. POSH expression in thyroid carcinoma.

FIGS. 16A-D show the immunohistochemistry of human tumor tissue sections: (A) Lymphoma (B) osteosacoma (C) liposarcoma (D) normal lung (left panel), lung carcinoma (right panel). POSH expression is detected in lymphoma, osteosarcoma, liposarcoma, and lung carcinoma.

FIG. 17. Effect of HPOSH on Gag-EGFP intracellular distribution.

FIG. 18. POSH is associated peripherally with the TGN membrane.

FIG. 19. Intracellular distribution of HIV-1 Nef in hPOSH-depleted cells.

FIG. 20. Intracellular distribution of Src in hPOSH-depleted cells.

FIG. 21. Intracellular distribution of Rapsyn in hPOSH-depleted cells.

FIG. 22. POSH reduction by siRNA abrogates West Nile virus infectivity.

FIG. 23. siRNA-mediated reduction of MSTP028 expression inhibits HIV virus-like particle production (Experiment 1).

FIG. 24. siRNA-mediated reduction of MSTP028 expression inhibits HIV virus-like particle production (Experiment 2).

### DETAILED DESCRIPTION OF THE INVENTION

1. Definitions

The term “aberrant process” refers to a process that is altered, modified, or different from the normal physiological process occurring in a host cell.

The term “binding” refers to a direct association between two molecules, due to, for example, covalent, electrostatic, hydrophobic, ionic and/or hydrogen-bond interactions under physiological conditions.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution“refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “envelop virus” as used herein refers to any virus that uses cellular membrane and/or any organelle membrane in the viral release process.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to“give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “intron” refers to a portion of nucleic acid that is intially transcribed into RNA but later removed such that it is not, for the most part, represented in the processed mRNA. Intron removal occurs through reactions at the 5′ and 3′ ends, typically referred to as 5′ and 3′ splice sites, respectively. Alternate use of different splice sites results in splice variants. An intron is not necessarily situated between two “exons”, or portions that code for amino acids, but may instead be positioned, for example, between the promoter and the first exon. An intron may be self-splicing or may require cellular components to be spliced out of the mRNA. A “heterologous intron” is an intron that is inserted into a coding sequence that is not naturally associated with that coding sequence. In addition, a heterologous intron may be a genrally natural intron wherein one or both of the splice sites have been altered to provide a desired quality, such as increased or descreased splice efficiency. Heterologous introns are often inserted, for example, to improve expression of a gene in a heterologous host, or to increase the production of one splice variant relative to another. As an example, the rabbit beta-globin gene may be used, and is commercially available on the pCI vector from Promega Inc. Other exemplary introns are provided in Lacy-Hulbert et al. (2001) Gene Ther 8(8):649-53.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

Lentiviruses include primate lentiviruses, e.g., human immunodeficiency virus types 1 and 2 (HIV-1/HIV-2); simian immunodeficiency virus (SIV) from Chimpanzee (SIVcpz), Sooty mangabey (SIVsmm), African Green Monkey (SIVagm), Syke's monkey (SIVsyk), Mandrill (SIVmnd) and Macaque (SIVmac). Lentiviruses also include feline lentiviruses, e.g., Feline immunodeficiency virus (FIV); Bovine lentiviruses, e.g., Bovine immunodeficiency virus (B); Ovine lentiviruses, e.g., Maedi/Visna virus (MVV) and Caprine arthritis encephalitis virus (CAEV); and Equine lentiviruses, e.g., Equine infectious anemia virus (EIAV). All lentiviruses express at least two additional regulatory proteins (Tat, Rev) in addition to Gag, Pol, and Env proteins. Primate lentiviruses produce other accessory proteins including Nef, Vpr, Vpu, Vpx, and Vif. Generally, lentiviruses are the causative agents of a variety of disease, including, in addition to immunodeficiency, neurological degeneration, and arthritis. Nucleotide sequences of the various lentiviruses can be found in Genbank under the following Accession Nos. (from J. M. Coffin, S. H. Hughes, and H. E. Varmus, “Retroviruses” Cold Spring Harbor Laboratory Press, 199,7 p 804): 1) HIV-1: K03455, M19921, K02013, M3843 1, M38429, K02007 and M17449; 2) HIV-2: M30502, J04542, M30895, J04498, M15390, M31113 and L07625; 3) SIV:M29975, M30931, M58410, M66437, L06042, M33262, M19499, M32741, M31345 and L03295; 4) FIV: M25381, M36968 and Ul 1820; 5)BIV. M32690; 6)EIAV: M16575, M87581 and U01866; 6)Visna: M10608, M51543, L06906, M60609 and M60610; 7) CAEV: M33677; and 8) Ovine lentivirus M31646 and M34193. Lentiviral DNA can also be obtained from the American Type Culture Collection (ATCC). For example, feline immunodeficiency virus is available under ATCC Designation No. VR-2333 and VR-3112. Equine infectious anemia virus A is available under ATCC Designation No. VR-778. Caprine arthritis-encephalitis virus is available under ATCC Designation No. VR-905. Visna virus is available under ATCC Designation No. VR-779. As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “maturation” as used herein refers to the production, post-translational processing, assembly and/or release of proteins that form a viral particle. Accrodingly, this includes the processing of viral proteins leading to the pinching off of nascent virion from the cell membrane.

A “membrane associated protein” is meant to include proteins that are integral membrane proteins as well as proteins that are stably associated with a membrane.

The term “p6” or p6gag” is used herein to refer to a protein comprising a viral L domain. Antibodies that bind to a p6 domain are referred to as “anti-p6 antibodies”. p6 also refers to proteins that comprise artificially engineered L domains including, for example, L domains comprising a series of L motifs.

The term “Gag protein” or “Gag polypeptide” refers to a polypeptide having Gag activity and preferably comprising an L (or late) domain. Exemplary Gag proteins include a motif such as PXXP, PPXY, RXXPXXP, RPDPTAP (SEQ ID NO: 163), RPLPVAP, (SEQ ID NO: 164), RPEPTAP (SEQ ID NO: 165), YEDL (SEQ ID NO: 166), PTAPPEY (SEQ ID NO: 167) and/or RPEPTAPPEE (SEQ ID NO: 168). HIV p24 is an exemplary Gag polypeptide. Gag-Pol proteins, such as the HIV p160 Gag-Pol are also Gag proteins

A “POSH nucleic acid” is a nucleic acid comprising a sequence as represented in any of SEQ ID NOS:1, 3, 4, 6, 8, and 10 as well as any of the variants described herein.

A “POSH polypeptide” or “POSH protein” is a polypeptide comprising a sequence as represented in any of SEQ ID NOS: 2, 5, 7, 9 and 11 as well as any of the variations described herein.

A “POSH-associated protein” or “POSH-AP” refers to a protein capable of interacting with and/or binding to a POSH polypeptide. Generally, the POSH-AP may interact directly or indirectly with the POSH polypeptide. Exemplary POSH-APs are provided throughout.

A “POSH binding protein” refers to a protein that binds to a POSH polypeptide. Examples of POSH binding proteins are Cbl-b, HERPUD1, GOCAP1, GOSR2, PRKAR1A, PKAR1, DDEF1, ARHV (CHP), SPG20, CENTB1, dynaminII, and RALA. Other POSH binding proteins are described throughout. Many POSH binding proteins bind to POSH in a yeast two-hybrid assay.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “receptor” or “protein having a receptor function” is a protein that interacts with an extracellular ligand or a ligand that is within the cell but in a space that is topologically equivalent to the extracellular space (eg. inside the Golgi, inside the endoplasmic reticulum, inside the nuclear membrane, inside a lysosome or transport vesicle, etc.). Exemplary receptors are identified herein by annotation as such in various public databases. Receptors often have membrane domains.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed-protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Certain RING domains are represented as amino acid sequences that are at least 80% identical to amino acids 12-52 of SEQ ID NO: 2 and is set forth in SEQ ID No: 26. Preferred RING domains are 85%, 90%, 95%, 98% and, most preferably, 100% identical to the amino acid sequence of SEQ ID NO: 26. Preferred RING domains of the invention bind to various protein partners to form a complex that has ubiquitin ligase activity. RING domains preferably interact with at least one of the following protein types: F box proteins, E2 ubiquitin conjugating enzymes and cullins.

The term “RNA interference” or “RNAi” refers to any method by which expression of a gene or gene product is decreased by introducing into a target cell one or more double-stranded RNAs which are homologous to the gene of interest (particularly to the messenger RNA of the gene of interest).

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

An “SH3” or “Src Homology 3” domain is a protein domain of generally about 60 amino acid residues first identified as a conserved sequence in the non-catalytic part of several cytoplasmic protein tyrosine kinases (e.g., Src, Abl, Lck). SH3 domains mediate assembly of specific protein complexes via binding to proline-rich peptides. Exemplary SH3 domains are represented by amino acids 137-192, 199-258, 448-505 and 832-888 of SEQ ID NO:2 and are set forth in SEQ ID Nos: 27-30. In certain embodiments, an SH3 domain interacts with a consensus sequence of RXaaXaaPXaaX6P (where X6, as defined in table 1 below, is a hydrophobic amino acid). In certain embodiments, an SH3 domain interacts with one or more of the following sequences: P(T/S)AP (SEQ ID NO: 169), PFRDY (SEQ ID NO: 170), RPEPTAP (SEQ ID NO: 165), RQGPKEP (SEQ ID NO: 171), RQGPKEPFR (SEQ ID NO: 172), RPEPTAPEE (SEQ ID NO: 168) and RPLPVAP (SEQ ID NO: 164).

As used herein, the term “specifically hybridizes” refers to the ability of a nucleic acid probe/primer of the invention to hybridize to at least 12, 15, 20, 25, 30, 35, 40, 45, 50 or 100 consecutive nucleotides of a POSH sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the POSH gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

“TGN-associated protein” as used herein includes a protein or polypeptide that localizes to the TGN; a protein or polypeptide that interacts either directly or indirectly with a protein or polypeptide that localizes to the TGN; and a protein or polypeptide that modulates the activity of a protein or polypeptide that localizes to the TGN.

“TGN-associated process” as used herein includes processing of proteins that takes place in the TGN; localization of proteins to the TGN; and trafficking or transport of proteins through the TGN.

“Disorders Associated with TGN-Associated Processes” as used herein include diseases which result from the aberrant processing of proteins at the TGN; aberrant localization of proteins to and/or from the TGN; and aberrant trafficking or transport of proteins through the TGN.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

As used herein, a “transgenic animal” is any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant human POSH protein. The “non-human animals” of the invention include vertebrates such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant is expressed in some but not all cells of the animal. The term “tissue specific chimeric animal” indicates that the recombinant human POSH genes is present and/or expressed in some tissues but not others.

As used herein, the term “transgene” means a nucleic acid sequence (encoding, e.g., human POSH polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity.

A “virion” is a complete viral particle; nucleic acid and capsid (and a lipid envelope in some viruses.

TABLE 1
Abbreviations for classes of amino acids*

| | | Amino Acids |
|---|---|---|
| Symbol | Category | Represented |
| X1 | Alcohol | Ser, Thr |
| X2 | Aliphatic | Ile, Leu, Val |
| Xaa | Any | Ala, Cys, Asp, Glu, Phe, |
| | | Gly, His, Ile, Lys, Leu, |
| | | Met, Asn, Pro, Gln, Arg, |
| | | Ser, Thr, Val, Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, |
| | | Ile, Lys, Leu, Met, Thr, |
| | | Val, Trp, Tyr |
| X7 | Negative | Asp, Glu |
| X8 | Polar | Cys, Asp, Glu, His, Lys, |
| | | Asn, Gln, Arg, Ser, Thr |
| X9 | Positive | His, Lys, Arg |
| X10 | Small | Ala, Cys, Asp, Gly, Asn, |
| | | Pro, Ser, Thr, Val |
| X11 | Tiny | Ala, Gly, Ser |
| X12 | Turnlike | Ala, Cys, Asp, Glu, Gly, |
| | | His, Lys, Asn, Gln, Arg, |
| | | Ser, Thr |
| X13 | Asparagine-Aspartate | Asn, Asp |

*Abbreviations as adopted from http://smart.emblheidelberg.de/SMART_DATA/alignments/consensus/grouping.html.

2. Overview

In certain embodiments, the present application relates to modulating one or more processes associated with the trans-Golgi network (TGN) by modulating a TGN-associated polypeptide. A TGN-associated polypeptide as described above includes within its scope a protein or polypeptide that localizes to the TGN; a protein or polypeptide or nucleic acid that interacts either directly or indirectly with a protein or polypeptide that localizes to the TGN; and a protein or polypeptide that modulates the activity of a protein or polypeptide that localizes to the TGN.

In certain embodiments, the TGN-associated protein may be one or more of a POSH polypeptide, a POSH-pathway polypeptide (a polypeptide involved in a signaling pathway associated with a POSH polypeptide), or a POSH-associated polypeptide (“POSH-AP”).

TGN-associated processes include the processing of proteins. Processing of these proteins includes post-translational modification of the protein, such as, e.g., gylcosylation, myristoylation, cleavage of one or more amino acids, and assembly of the protein into a protein complex. TGN-associated processes also include the localization and trafficking of proteins through the TGN, including the trafficking of proteins from the endoplasmic reticulum (ER) to the plasma membrane or to lysosomes, both enterograde and retrograde. Proteins processed by the TGN include viral, bacterial, parasitic, and other microbial proteins. In certain aspects, the application relates to modulation of a TGN-associated protein complex. In certain embodiments, the TGN-associated protein complex comprises at least one TGN-associated protein. In further embodiments, the TGN-associated protein complex further comprises nucleic acid.

In certain aspects, the application relates to modulation of intracellular transport of myristoylated proteins from the TGN. N-myristoylation is an acylation process, which results in covalent attachment of myristate, a 14-carbon saturated fatty acid to the N-terminal glycine of proteins (Farazi et al., J. Biol. Chem. 276: 39501-04 (2001)). N-myristoylation occurs co-translationaly and promotes weak and reversible protein-membrane interaction. Myristoylated proteins are found both in the cytoplasm and associated with membrane. Membrane association is dependent on protein configuration, i.e., surface accessibility of the myristoyl group may be regulated by protein modifications, such as phosphorylation, ubiquitination etc. Modulation of intracellular transport of myristoylated proteins in the application includes effects on transport and localization of these modified proteins.

Transport of proteins from the TGN includes different pathways and distinct destinations. It includes both constitutive and regulated transport and secretion of proteins. The cargo proteins themselves contain the sorting signals that drive their segregation, which interact with assembly or adaptor protein complexes. This specific interaction leads to the incorporation of a subset of proteins into transport vesicles. The absence or mutation in an adaptor or regulator of TGN sorting and transport will result in either block in transport or in missorting of a protein cargo to a different intracellular location. Certain aspects of the application relate to POSH, POSH-pathway, and/or a POSH-AP polypeptide-regulated transport of proteins from the TGN to the plasma membrane.

In certain aspects, the application relates to modulation of a TGN-associated process that is related to viral maturation. Modulation of a TGN-associated process includes inhibition or potentiation of a TGN-associated process that results in inhibition or prevention of viral maturation. TGN-associated processes include processing of viral proteins. Processing of viral proteins by the TGN encompasses assembly of viral proteins in the TGN. Processing of viral proteins additionally includes the transport of viral proteins from the TGN to the plasma membrane. TGN-associated processes also include the processing of viral nucleic acid (e.g., viral RNA) or ribonucleoparticles. In certain embodiments of the application, TGN-associated processes associated with viral maturation are regulated by one or more of a POSH, a POSH-pathway and/or a POSH-AP polypeptide.

In certain aspects, the invention relates to modulation of a TGN-associated process that is correlated with a neurological disorder. Modulation of a TGN-associated process includes inhibition or potentiation of a TGN-associated process that results in mitigation, inhibition, or prevention of a neurological disorder. Neurological disorders associated with TGN-associated processes include Alzheimer's disease, Parkinson's disease, Huntington's disease, schizophrenia, Niemann-Pick's disease, and prion-associated diseases.

In certain embodiments of the application, TGN-associated processes associated with a neurological disorder are regulated by one or more of a POSH, a POSH-pathway and/or a POSH-AP polyp eptide. TGN-associated processes correlated with a neurological disorder include the aberrant processing of proteins that are in the TGN or are affiliated with the TGN. Aberrant processing includes an increase or decrease of a protein in the TGN in comparison to the amount of the protein present in the TGN under normal physiological conditions. Aberrant processing also includes incomplete processing of a protein in the TGN, such as the incomplete proteolysis of a precursor protein. An exemplary protein aberrantly processed in the TGN is the beta amyloid precursor protein, which aberrant processing is associated with Alzheimer's disease. In certain embodiments of the invention, the improper processing of beta amyloid polypeptides involves the POSH-AP, HERPUD1. In certain aspects of the application, aberrant processing of proteins in the TGN includes improper post-translational modification of the protein, such as improper glycosylation of the protein. Improper glycosylation includes improper N-linked glycosylation and improper O-linked glycosylation. In certain aspects, the application relates to the incorrect processing of proteins in the TGN whereby the protein is incorrectly sorted to or by the TGN and/or improperly transported from the TGN.

In certain aspects, the invention relates to modulation of a TGN-associated process that is correlated with a neoplastic condition. Modulation of a TGN-associated process includes inhibition or potentiation of a TGN-associated process that results in mitigation, inhibition, or prevention of a neoplastic condition, such as cancer. Cancers associated with TGN-associated processes include thyroid carcinoma, liver cancer (hepatocellular cancer), lung cancer, cervical cancer, colorectal cancer, ovarian cancer, renal cell carcinoma, lymphoma, osteosarcoma, prostate cancer, liposarcoma, leukemia, breast carcinoma, and breast adeno-carcinoma.

In certain embodiments of the application, TGN-associated processes associated with a neoplastic condition are regulated by one or more of a POSH, a POSH-pathway and/or a POSH-AP polypeptide. TGN-associated processes associated with a neoplastic condition include the processing of proteins in a cancer cell, e.g., proteins that are in the TGN or are affiliated with the TGN. TGN-associated processes associated with a neoplastic condition also include the aberrant processing of proteins that are in the TGN or are affiliated with the TGN. Aberrant processing includes an increase or decrease of a protein in the TGN in comparison to the amount of the protein present under normal physiological conditions. Aberrant processing also includes incomplete processing of a protein in the TGN, such as the incomplete proteolysis of a precursor protein. In certain aspects of the application, aberrant processing of proteins in the TGN includes improper post-translational modification of the protein, such as improper glycosylation of the protein. In certain aspects, the application relates to the incorrect processing of proteins in the TGN whereby the protein is incorrectly sorted to or by the TGN and/or improperly transported from the TGN.

In certain aspects, the application relates to the aberrant processing of a protein associated with a neoplastic condition whereby the aberrant processing of the protein results in an increased or decreased level of transport of the protein from the TGN to the plasma membrane in comparison to the level of transport of the protein under normal physiological conditions. Such aberrant processing includes an increase or decrease in the secretion of a protein. An exemplary aberrantly processed protein whose secretion profile is altered is PLD. In certain embodiments of the application, PLD secretion is effected by the activity of a POSH polypeptide.

In certain aspects, the application relates to modulation of a TGN-associated process that is correlated with an immunological condition, such as an inflammatory disease or an autoimmune disorder (e.g., diabetes).

In certain aspects, the application relates to modulation of a TGN-associated process that is associated with a cardiovascular disease, such as myocardial infarction or stroke.

In certain embodiments of the application, the application relates to modulation of a TGN-associated protein. Exemplary TGN-associated proteins include POSH; the POSH-APs Vpu, HERPUD1, GOCAP1, GOSR2, PKA, and Cbl-b; and the POSH-pathway polypeptides, PLD and dynamin II. In one embodiment, the application relates to the modulation of the POSH-AP, PKA. PKA has been found to play a regulatory role in the production of constitutive transport vesicles from the TGN (see, for example, Muniz, M et al (1997) Proc. Natl. Acad. Sci. USA 94:14461-14466). Accordingly, modulation of PKA activity may affect a TGN-associated process such as, for example, the aberrant secretion of a TGN-associated protein. In certain aspects, modulation of PKA activity may affect the secretion of the POSH-pathway polypeptide, PLD, which is a TGN-associated protein.

In certain embodiments of the application, the application relates to modulation of TGN-associated proteins that are localized to the TGN by the presence of a TGN-localization signal within the polypeptide. The TGN-localization domain may be internal, C-terminal or N-terminal or may be present as part of a fusion protein or other linkage comprising the TGN-associated polypeptide. TGN-localization domains include dileucine motifs, acidic amino acid clusters (e.g., SDSEEDE), and tyrosine-based motifs. An exemplary tyrosine-based motif is YXXO, wherein the O is any amino acid comprising a bulky, hydrophobic group. Another example of a TGN-localization domain that is a tyrosine-based motif is the peptide sequence, YKGL. Additional TGN-localization domains include a casein kinase II phosphorylation site, a GAT domain, a gamma ear domain, a GRIP domain, an ENTH domain, a cysteine rich domain, and a granin motif (e.g., ESLALEELEL).

In certain aspects, the application relates to the finding that Gag protein from HIV assembles in the trans Golgi network and is then transported to the plasma membrane, and further, that disruption of a polypeptide involved in this process, such as the POSH polypeptide, inhibits viral maturation. The application also relates to the findings that human POSH is localized to the trans Golgi network, that depletion of POSH by siRNA prevents viral budding from the cell surface and causes instead budding at internal membranes, that human POSH participates in the trans Golgi trafficking of additional host cell polypeptides and that human POSH operates, at least in part, independent of the L-domain of HIV Gag.

In certain aspects, the invention relates to novel human TGN-associated nucleic acids and proteins, and related methods and compositions. In certain aspects, the invention relates to novel associations between certain disease states and TGN-associated nucleic acids and proteins. TGN-associated polypeptides intersect with and regulate a wide range of key cellular functions that may be manipulated by affecting the level of and/or activity of TGN-associated polypeptides. In certain aspects, by identifying the human POSH gene the present invention provides methods for identifying diseases that are associated with defects in the POSH gene and methods for ameliorating such diseases. In further aspects, the invention provides nucleic acid agents (e.g., RNAi probes, antisense), antibody-related agents, small molecules and other agents that affect TGN-associated protein function. In further aspects, the invention provides methods for identifying agents that affect TGN-associated protein function, and the function of proteins that associate with TGN-associated polypeptides and/or participate in a TGN-associated polypeptide-mediated process. Other aspects and embodiments are described herein.

In certain aspects, the invention relates to the discovery that certain TGN-associated proteins, such as POSH polypeptides, function as E3 enzymes in the ubiquitination system. Accordingly, downregulation or upregulation of TGN-associated protein ubiquitin ligase activity can be used to manipulate biological processes that are affected by protein ubiquitination. Downregulation or upregulation may be achieved at any stage of TGN-associated protein formation and regulation, including transcriptional, translational or post-translational regulation. For example, POSH transcript levels may be decreased by RNAi targeted at a POSH gene sequence. As another example, POSH ubiquitin ligase activity may be inhibited by contacting POSH with an antibody that binds to and interferes with a POSH RING domain or a domain of POSH that mediates interaction with a target protein (a protein that is ubiquitinated at least in part because of POSH activity). As another example, a TGN-associated protein activity may be increased by causing increased expression of the TGN-associated protein or an active portion thereof. A ubiquitin ligase, such as POSH, may participate in biological processes including, for example, one or more of the various stages of a viral lifecycle, such as viral entry into a cell, production of viral proteins, assembly of viral proteins and release of viral particles from the cell, or one or more of the various stages of cell transformation. A TGN-associated protein may participate in diseases characterized by the accumulation of ubiquitinated proteins, such as dementias (e.g., Alzheimer's and Pick's), inclusion body myositis and myopathies, polyglucosan body myopathy, and certain forms of amyotrophic lateral sclerosis. TGN-associated proteins may participate in diseases characterized by the excessive or inappropriate ubiquitination and/or protein degradation. In addition, TGN-associated proteins may participate in oncological processes, such as the failure of cell division control systems, the failure of cell death regulatory systems, and the failure to downregulate hyperactive oncogenes, such as hyperactive membrane-bound growth factor receptors. By identifying certain TGN-associated polypeptides as ubiquitin ligases, aspects of the present invention permit one of ordinary skill in the art to identify diseases that are associated with an altered TGN-associated protein ubiquitin ligase activity.

In certain aspects, the invention relates to the discovery that certain TGN-associated protein polypeptides are involved in viral maturation, including the production, post-translational processing, assembly and/or release of proteins in a viral particle. Accordingly, viral infections may be ameliorated by inhibiting an activity (e.g., ubiquitin ligase activity or target protein interaction) of a TGN-associated protein, and in preferred embodiments, the virus is a retroid virus, an RNA virus and an envelop virus, including HIV, Ebola, HBV, HCV and HTLV. Additional viral species are described in greater detail below. In certain instances, a decrease of a TGN-associated protein function is lethal to cells infected with a virus that employs a TGN-associated protein in release of viral particles. While not wishing to be bound to mechanism, it appears that loss of a TGN-associated protein function, such as POSH function, in such cells leads to cell death through an overaccumulation of viral particles, or portions thereof, in the cell. In certain embodiments, the inhibition of a TGN-associated protein activity, e.g., by siRNA knockdown may be used to destroy infected cells, even cells with nearly latent virus, because such cells will die from eventual overaccumulation of viral particles or portions thereof.

In certain aspects, the invention relates to the discovery that TGN-associated proteins, such as human POSH, interact with Rac, a small GTPase. Rho, Rac and Cdc42 operate together to regulate organization of the actin cytoskeleton and the JNK MAP kinase pathway. Ectopic expression of mouse POSH (“mPOSH”) activates the JNK pathway and causes nuclear localization of NF-κB. Overexpression of mPOSH in fibroblasts stimulates apoptosis. (Tapon et al. (1998) EMBO J. 17:1395-404). In Drosophila, POSH may interact, or otherwise influence the signaling of, another GTPase, Ras. (Schnorr et al. (2001) Genetics 159: 609-22). The JNK pathway and NF-κB regulate a variety of key genes involved in, for example, immune responses, inflammation, cell proliferation and apoptosis. For example, NF-κB regulates the production of interleukin 1, interleukin 8, tumor necrosis factor and many cell adhesion molecules. NF-κB has both pro-apoptotic and anti-apoptotic roles in the cell (e.g., in FAS-induced cell death and TNF-alpha signaling, respectively). NF-κB is negatively regulated, in part, by the inhibitor proteins IκBα and IκBβ (collectively termed “IκB”). Phosphorylation of IκB permits activation and nuclear localization of NF-κB. Phosphorylation of IκB triggers its degradation by the ubiquitin system. Accordingly, in yet another embodiment, a TGN-associated polypeptide stimulates the JNK pathway. In an additional embodiment, a TGN-associated polypeptide promotes nuclear localization of NF-κB. In an additional embodiment, a TGN-associated protein modulates the activity of a POSH-pathway polypeptide such as for example a Chp polypeptide. In another embodiment, a TGN-associated protein modulates the activity of a POSH-pathway polypeptide such as for example a SRK polypeptide. In further embodiments, manipulation of TGN-associated protein levels and/or activities may be used to manipulate apoptosis. By upregulating TGN-associated proteins, apoptosis may be stimulated in certain cells, and this will generally be desirable in conditions characterized by excessive cell proliferation (e.g., in certain cancers). By downregulating TGN-associated proteins, apoptosis may be diminished in certain cells, and this will generally be desirable in conditions characterized by excessive cell death, such as myocardial infarction, stroke, degenerative diseases of muscle and nerve, and for organ preservation prior to transplant.

In a further embodiment, a TGN-associated polypeptide associates with a vesicular trafficking complex, such as a clathrin- or coatomer-containing complex, and particularly a trafficking complex that localizes to the Golgi apparatus.

3. Exemplary Nucleic Acids and Expression Vectors

In certain aspects, the methods of the invention provide nucleic acids encoding TGN-associated polypeptides. TGN-associated polypeptides may include POSH-pathway and POSH-AP polypeptides. The term POSH-pathway polypeptide is used herein to refer to various naturally occurring POSH-pathway homologs, as well as functionally similar variants and fragments that retain at least 80%, 90%, 95%, or 99% sequence identity to a naturally occurring POSH-pathway polypeptides. The term POSH-AP polypeptide is used herein to refer to various naturally occurring POSH-AP homologs, as well as functionally similar variants and fragments that retain at least 80%, 90%, 95%, or 99% sequence identity to a naturally occurring POSH-AP polypeptides.

In certain aspects, the methods of the invention provide nucleic acids encoding POSH-AP polypeptides, such as, for example, SEQ ID NOS: 37-73 and 98-115, and POSH-pathway polypeptides, such as, for example, SEQ ID NO: 74. Nucleic acids of the invention are further understood to include nucleic acids that comprise variants of SEQ ID NOS: 37-74 and 98-115. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NOS: 37-74 and 98-115, e.g., due to the degeneracy of the genetic code. In other embodiments, variants will also include sequences that will hybridize under highly stringent conditions to a nucleotide sequence of a coding sequence designated in any of SEQ ID NOS: 37-74 or 98-115. Preferred nucleic acids employed by methods of the invention are POSH-AP and POSH-pathway nucleic acid sequences, including, for example, any of SEQ ID NOS: 37-74 and 98-115 and variants thereof and nucleic acids encoding an amino acid sequence selected from among SEQ ID NOS: 75-97 and 116-125.

In certain aspects, the methods of the invention provide nucleic acids encoding POSH polypeptides, such as, for example, SEQ ID NOS: 2, 5, 7, 9, 11, 26, 27, 28, 29 and 30. Nucleic acids of the invention are further understood to include nucleic acids that comprise variants of SEQ ID NOS: 1,3, 4, 6, 8, 10,31, 32, 33, 34, and 35. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID Nos: 1, 3, 4, 6, 8 10, 31, 32, 33, 34, and 35, e.g., due to the degeneracy of the genetic code. In other embodiments, variants will also include sequences that will hybridize under highly stringent conditions to a nucleotide sequence of a coding sequence designated in any of SEQ ID Nos: 1, 3, 4, 6, 8 10, 31, 32, 33, 34, and 35. Preferred nucleic acids of the invention are human POSH sequences, including, for example, any of SEQ ID Nos: 1, 3, 4, 6, 31, 32, 33, 34, 35 and variants thereof and nucleic acids encoding an amino acid sequence selected from among SEQ ID Nos: 2, 5, 7, 26, 27, 28, 29 and 30.

One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from SEQ ID Nos:1, 3, 4, 6, 8, 10, 31, 32, 33, 34, 35 and 37-74 due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

Optionally, a TGN-associated nucleic acid of the invention will genetically complement a partial or complete TGN-associated protein loss of function phenotype in a cell. For example, a POSH nucleic acid of the invention may be expressed in a cell in which endogenous POSH has been reduced by RNAi, and the introduced POSH nucleic acid will mitigate a phenotype resulting from the RNAi. An exemplary POSH loss of function phenotype is a decrease in virus-like particle production in a cell transfected with a viral vector, optionally an HIV vector. In certain embodiments, a POSH nucleic acid, when expressed at an effective level in a cell, induces apoptosis.

Another aspect of the invention relates to TGN-associated nucleic acids that are used for antisense, RNAi or ribozymes. As used herein, nucleic acid therapy refers to administration or in situ generation of a nucleic acid or a derivative thereof which specifically hybridizes (e.g., binds) under cellular conditions with the cellular mRNA and/or genomic DNA encoding one of the subject TGN-associated polypeptides so as to inhibit production of that protein, e.g., by inhibiting transcription and/or translation. The binding may be by conventional base pair complementarity, or, for example, in the case of binding to DNA duplexes, through specific interactions in the major groove of the double helix.

A nucleic acid therapy construct of the present invention can be delivered, for example, as an expression plasmid which, when transcribed in the cell, produces RNA which is complementary to at least a unique portion of the cellular mRNA which encodes a TGN-associated protein. Alternatively, the construct is an oligonucleotide which is generated ex vivo and which, when introduced into the cell causes inhibition of expression by hybridizing with the mRNA and/or genomic sequences encoding a TGN-associated protein. Such oligonucleotide probes are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., (1988) Cancer Res 48:2659-2668.

Accordingly, the modified oligomers of the invention are useful in therapeutic, diagnostic, and research contexts. In therapeutic applications, the oligomers are utilized in a manner appropriate for nucleic acid therapy in general.

In addition to use in therapy, the oligomers of the invention may be used as diagnostic reagents to detect the presence or absence of the TGN-associated DNA or RNA sequences to which they specifically bind, such as for determining the level of expression of a gene of the invention or for determining whether a gene of the invention contains a genetic lesion.

In another aspect of the invention, the subject nucleic acid is provided in an expression vector comprising a nucleotide sequence encoding a subject TGN-associated protein and operably linked to at least one regulatory sequence. Regulatory sequences are art-recognized and are selected to direct expression of the TGN-associated protein. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding TGN-associated protein. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject TGN-associated proteins in cells propagated in culture, e.g., to produce proteins or polypeptides, including fusion proteins or polypeptides, for purification.

This invention also pertains to a host cell transfected with a recombinant gene including a coding sequence for one or more of the subject TGN-associated proteins. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject TGN-associated proteins. For example, a host cell transfected with an expression vector encoding a TGN-associated polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the TGN-associated protein is a fusion protein containing a domain which facilitates its purification, such as a TGN-associated protein-GST fusion protein, -intein fusion protein, -cellulose binding domain fusion protein, -polyhistidine fusion protein, etc.

A nucleotide sequence encoding a TGN-associated protein can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant TGN-associated nucleic acid can be produced by ligating the cloned gene, or a portion thereof, into a vector suitable for expression in either prokaryotic cells, eukaryotic cells, or both. Expression vehicles for production of a recombinant TGN-associated protein include plasmids and other vectors. For instance, suitable vectors for the expression of a TGN-associated protein include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant POSH, POSH-pathway, or POSH-AP polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of a TGN-associated protein. For example, the VP6 capsid protein of rotavirus can be used as an immunologic carrier protein for portions of polypeptide, either in the monomeric form or in the form of a viral particle. The nucleic acid sequences corresponding to the portion of the TGN-associated protein to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of a TGN-associated protein and the poliovirus capsid protein can be created to enhance immunogenicity (see, for example, EP Publication NO: 0259149; and Evans et al.,, (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of a TGN-associated protein is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of a TGN-associated protein can also be expressed and presented by bacterial cells.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified TGN-associated protein (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

TABLE 2
Exemplary POSH nucleic acids

| | | Accession |
|---|---|---|
| Sequence Name | Organism | Number |
| cDNA FLJ11367 fis, clone | Homo sapiens | AK021429 |
| HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 3
Exemplary POSH polypeptides

| Sequence Name | Organism | Accession Number |
|---|---|---|
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

In addition the following Tables provide the nucleic acid sequence and related SEQ ID NOs for domains of humans POSH protein and a summary of sequence identification numbers used in this application.

TABLE 4

Nucleic Acid Sequences and related SEQ ID NOs
for domains in human POSH

| Name of the | | SEQ ID |
|---|---|---|
| sequence | Sequence | NO. |
| RING domain | TGTCCGGTGTGTCTAGAGCGCCTTGATGCTTC | 31 |
| | TGCGAAGGTCTTGCCTTGCCAGCATACGTTTT | |
| | GCAAGCGATGTTTGCTGGGGATCGTAGGTTCT | |
| | CGAAATGAACTCAGATGTCCCGAGT | |
| 1^{st} SH₃ | CCATGTGCCAAAGCGTTATACAACTATGAAGG | 32 |
| domain | AAAAGAGCCTGGAGACCTTAAATTCAGCAAAG | |
| | GCGACATCATCATTTTGCGAAGACAAGTGGAT | |
| | GAAAATTGGTACCATGGGGAAGTCAATGGAAT | |
| | CCATGGCTTTTTCCCCACCAACTTTGTGCAGA | |
| | TTATT | |
| 2^{nd} SH₃ | CCTCAGTGCAAAGCACTTTATGACTTTGAAGT | 33 |
| domain | GAAAGACAAGGAAGCAGACAAAGATTGCCTTC | |
| | CATTTGCAAAGGATGATGTTCTGACTGTGATC | |
| | CGAAGAGTGGATGAAAACTGGGCTGAAGGAAT | |
| | GCTGGCAGACAAAATAGGAATATTTCCAATTT | |
| | CATATGTTGAGTTTAAC | |
| 3^{rd} SN₃ | AGTGTGTATGTTGCTATATATCCATACACTCC | 34 |
| domain | TCGGAAAGAGGATGAACTAGAGCTGAGAAAAG | |
| | GGGAGATGTTTTTAGTGTTTGAGCGCTGCCAG | |
| | GATGGCTGGTTCAAAGGGACATCCATGCATAC | |
| | CAGCAAGATAGGGGTTTTCCCTGGCAATTATG | |
| | TGGCACCAGTC | |
| 4^{th} SN₃ | GAAAGGCACAGGGTGGTGGTTTCCTATCCTCC | 35 |
| domain | TCAGAGTGAGGCAGAACTTGAACTTAAAGAAG | |
| | GAGATATTGTGTTTGTTCATAAAAAACGAGAG | |
| | GATGGCTGGTTCAAAGGCACATTACAACGTAA | |
| | TGGGAAAACTGGCCTTTTCCCAGGAAGCTTTG | |
| | TGGAAAACA | |

TABLE 5
Summary of Sequence Identification Numbers

| | Sequence |
|---|---|
| | Identification Number |
| Sequence Information | (SEQ ID NO) |
| Human POSH Coding Sequence | SEQ ID No: 1 |
| Human POSH Amino Acid Sequence | SEQ ID No: 2 |
| Human POSH cDNA Sequence | SEQ ID No: 3 |
| 5′ cDNA Fragment of Human POSH | SEQ ID No: 4 |
| N-terminus Protein Fragment of | SEQ ID No: 5 |
| Human POSH | |
| 3′ mRNA Fragment of Human POSH | SEQ ID No: 6 |
| C-terminus Protein Fragment of | SEQ ID No: 7 |
| Human POSH | |
| Mouse POSH mRNA Sequence | SEQ ID No: 8 |
| Mouse POSH Protein Sequence | SEQ ID No: 9 |
| Drosophila melanogaster POSH | SEQ ID No: 10 |
| mRNA Sequence | |
| Drosophila melanogaster POSH | SEQ ID No: 11 |
| Protein Sequence | |
| Human POSH RING Domain Amino | SEQ ID No: 26 |
| Acid Sequence | |
| Human POSH 1^{st} SH₃ Domain Amino | SEQ ID No: 27 |
| Acid Sequence | |
| Human POSH 2^{nd} SH₃ Domain Amino | SEQ ID No: 28 |
| Acid Sequence | |
| Human POSH 3^{rd} SH₃ Domain Amino | SEQ ID No: 29 |
| Acid Sequence | |
| Human POSH 4^{th} SH₃ Domain Amino | SEQ ID No: 30 |
| Acid Sequence | |
| Human POSH RING Domain Nucleic | SEQ ID No: 31 |
| Acid Sequence | |
| Human POSH 1^{st} SH₃ Domain Nucleic | SEQ ID No: 32 |
| Acid Sequence | |
| Human POSH 2^{nd} SH₃ Domain Nucleic | SEQ ID No: 33 |
| Acid Sequence | |
| Human POSH 3^{rd} SH₃ Domain Nucleic | SEQ ID No: 34 |
| Acid Sequence | |
| Human POSH 4^{th} SH₃ Domain Nucleic | SEQ ID No: 35 |
| Acid Sequence | |

4. Exemplary Polypeptides

In certain aspects, the methods of the invention also make available isolated and/or purified forms of the subject TGN-associated proteins, which may include POSH-pathway and POSH-AP polypeptides, which are isolated from, or otherwise substantially free of, other intracellular proteins which might normally be associated with the protein or a particular complex including the protein. The term POSH-pathway polypeptide is used herein to refer to various naturally occurring POSH-pathway homologs, as well as functionally similar variants and fragments that retain at least 65%, 70%, 75%, 80%, 85%, 90%, 95%, 97%, 98%, 99% or 100% sequence identity to a naturally occurring POSH-pathway polypeptide. The term POSH-AP polypeptide is used herein to refer to various naturally occurring POSH-AP homologs, as well as functionally similar variants and fragments that retain at least 65%, 70%, 75%, 80%, 85%, 90%, 95%, 97%, 98%, 99% or 100% sequence identity to a naturally occurring POSH-AP polypeptide.

The methods of the present invention also make available isolated and/or purified forms of the subject TGN-associated proteins, which may be POSH-AP or POSH-pathway polypeptides, which are isolated from, or otherwise substantially free of, other intracellular proteins which might normally be associated with the protein or a particular complex including the protein. In certain embodiments, POSH-AP and POSH-pathway polypeptides have an amino acid sequence that is at least 60% identical to an amino acid sequence as set forth in any of SEQ ID NOS: 75-97 and 116-125. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOS: 75-97 and 116-125.

The methods of the present invention also make available isolated and/or purified forms of the subject TGN-associated proteins, which may be POSH polypeptides, which are isolated from, or otherwise substantially free of, other intracellular proteins which might normally be associated with the protein or a particular complex including the protein. In certain embodiments, POSH polypeptides have an amino acid sequence that is at least 60% identical to an amino acid sequence as set forth in any of SEQ ID Nos: 2, 5, 7, 9, 11, 26, 27, 28, 29 and 30. In other embodiments, the polypeptide has an amino acid sequence at least 65%, 70%, 75%, 80%, 85%, 90%, 95%, 97%, 98%, 99% or 100% identical to an amino acid sequence as set forth in any of SEQ ID NOS: 2, 5, 7, 9, 11, 26, 27, 28, 29 and 30.

Optionally, a TGN-associated protein of the invention will function in place of a respective endogenous TGN-associated protein, for example by mitigating a respective partial or complete TGN-associated protein loss of function phenotype in a cell. For example, a TGN-associated protein of the invention may be produced in a cell in which endogenous TGN-associated protein has been reduced by RNAi, and the introduced TGN-associated polypeptide will mitigate a phenotype resulting from the RNAi. An exemplary TGN-associated protein loss of function phenotype is a POSH loss of function phenotype that is a decrease in virus-like particle production in a cell transfected with a viral vector, optionally an HIV vector. In certain embodiments, a TGN-associated polypeptide, when produced at an effective level in a cell, induces apoptosis. In certain embodiments, a TGN-associated polypeptide, when produced at an effective level in a cell, inhibits the progression of a neurological disorder.

In certain embodiments, a TGN-associated protein polypeptide of the invention interacts with a viral Gag protein. In additional embodiments, TGN-associated protein polypeptides may also, or alternatively, function in ubiquitination in part through the activity of a RING domain.

In another aspect, the invention provides polypeptides that are agonists or antagonists of a TGN-associated protein. Variants and fragments of a TGN-associated protein may have a hyperactive or constitutive activity, or, alternatively, act to prevent TGN-associated protein from performing one or more functions. For example, a truncated form lacking one or more domain may have a dominant negative effect.

Another aspect of the invention relates to polypeptides derived from a full-length TGN-associated protein. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of the formation of a specific protein complex, or more generally of a TGN-associated protein complex, such as by microinjection assays.

It is also possible to modify the structure of the subject TGN-associated proteins for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the TGN-associated proteins described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of a TGN-associated protein can be assessed, e.g., for their ability to bind to another polypeptide, e.g., another TGN-associated protein or another protein involved in viral maturation, cell transformation, cell proliferation, or the progression of a neurological disorder. Polypeptides in which more than one replacement has taken place can readily be tested in the same manner.

This invention further contemplates a method of generating sets of combinatorial mutants of the subject TGN-associated proteins, as well as truncation mutants, and is especially useful for identifying potential variant sequences (e.g., homologs) that are functional in binding to a TGN-associated protein. The purpose of screening such combinatorial libraries is to generate, for example, TGN-associated protein homologs which can act as either agonists or antagonist, or alternatively, which possess novel activities all together. Combinatorially-derived homologs can be generated which have a selective potency relative to a naturally occurring TGN-associated protein. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the TGN-associated protein of interest. Such homologs, and the genes which encode them, can be utilized to alter TGN-associated protein levels by modulating the half-life of the protein. For instance, a short half-life can give rise to more transient biological effects and, when part of an inducible expression system, can allow tighter control of recombinant TGN-associated protein levels within the cell. As above, such proteins, and particularly their recombinant nucleic acid constructs, can be used in gene therapy protocols.

In similar fashion, TGN-associated protein homologs can be generated by the present combinatorial approach to act as antagonists, in that they are able to interfere with the ability of the corresponding wild-type protein to function.

In a representative embodiment of this method, the amino acid sequences for a population of TGN-associated protein homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential TGN-associated protein sequences. For instance, a mixture of synthetic oligonucleotides can be enzymatically ligated into gene sequences such that the degenerate set of potential TGN-associated protein nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential TGN-associated protein sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. A G Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404406; Cwirla et al., (1990) PNAS USA 87: 6378-6382; as well as U.S. Pat. Nos: 5,223,409, 5,198,346, and 5,096,815).

Alternatively, other forms of mutagenesis can be utilized to generate a combinatorial library. For example, TGN-associated protein homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of TGN-associated protein.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of TGN-associated protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind a TGN-associated protein is detected in a “panning assay”. For instance, a library of TGN-associated protein variants can be cloned into the gene for a surface membrane protein of a bacterial cell (Ladner et al.,, WO 88/06630; Fuchs et al., (1991) Bio/Technology 9:1370-1371; and Goward et al., (1992) TIBS 18:136-140), and the resulting fusion protein detected by panning, e.g., using a fluorescently labeled molecule which binds the TGN-associated polypeptide, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject TGN-associated proteins to generate mimetics, e.g., peptide or non-peptide agents, which are able to mimic binding of the authentic protein to another cellular partner. Such mutagenic techniques as described above, as well as the thioredoxin system, are also particularly useful for mapping the determinants of a TGN-associated protein which participate in protein-protein interactions involved in, for example, binding of proteins involved in viral maturation to each other. To illustrate, the critical residues of a TGN-associated protein which are involved in molecular recognition of a substrate protein can be determined and used to generate TGN-associated protein-derived peptidomimetics which bind to the substrate protein, and by inhibiting TGN-associated protein binding, act to inhibit its biological activity. By employing, for example, scanning mutagenesis to map the amino acid residues of a TGN-associated protein which are involved in binding to another polypeptide, peptidomimetic compounds can be generated which mimic those residues involved in binding. For instance, non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

The following table provides the sequences of the RING domain and the various SH3 domains.

TABLE 6

Amino Acid Sequences and related SEQ ID NOs
for domains in human POSH

| Name of | | |
|---|---|---|
| the | | SEQ ID |
| sequence | Sequence | NO. |
| RING | CPVCLERLDASAKVLPCQHTFCKRCLLGIVGSRNEL | 26 |
| domain | RCPEC | |
| 1^{st} SH₃ | PCAKALYNYEGKEPGDLKFSKGDIIILRRQVDENWY | 27 |
| domain | HGEVNGIHGFFPTNFVQIIK | |
| 2^{nd} SH₃ | PQCKALYDFEVKDKEADKDCLPFAKDDVLTVIRRVD | 28 |
| domain | ENWAEGMLADKIGIFPISYVEFNS | |
| 3^{rd} SH₃ | SVYVAIYPYTPRKEDELELRKGEMFLVFERCQDGWF | 29 |
| domain | KGTSMHTSKIGVFPGNYVAPVT | |
| 4^{th} SH₃ | ERHRVVVSYPPQSEAELELKEGDIVFVHKKREDGWF | 30 |
| domain | KGTLQRNGKTGLFPGSFVENI | |

5. Antibodies and Uses Thereof

Another aspect of the invention pertains to an antibody specifically reactive with a TGN-associated protein. For example, by using immunogens derived from a TGN-associated protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a TGN-associated polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a TGN-associated protein can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a TGN-associated protein of a mammal, e.g., antigenic determinants of a protein set forth in any one of SEQ ID NOS: 2, 75-97, and 116-125.

In one embodiment, antibodies are specific for a RING domain or an SH3 domain, and preferably the domain is part of a TGN-associated protein. In a more specific embodiment, the domain is part of an amino acid sequence set forth in SEQ ID NO:2. In a set of exemplary embodiments, an antibody binds to one or more SH3 domains represented by amino acids 137-192 of SEQ ID NO:2, amino acids 199-258 of SEQ ID NO:2, amino acids 448-505 of SEQ ID NO:2, and/or amino acids 832-888 of SEQ ID NO:2. In another exemplary embodiment, an antibody binds to a RING domain represented by amino acids 12-52 of SEQ ID NO:2. In another embodiment, the antibodies are immunoreactive with one or more proteins having an amino acid sequence that is at least 80% identical to an amino acid sequence as set forth in any one of SEQ ID NOS: 2, 75-97, and 116-125. In other embodiments, an antibody is immunoreactive with one or more proteins having an amino acid sequence that is 85%, 90%, 95%, 98%, 99% or identical to an amino acid sequence as set forth in any one of SEQ ID NOS: 2, 75-97, and 116-125.

Following immunization of an animal with an antigenic preparation of a TGN-associated protein, anti-TGN-associated protein antisera can be obtained and, if desired, polyclonal anti-TGN-associated protein antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian TGN-associated protein of the present invention and monoclonal antibodies isolated from a culture comprising such hybridoma cells. In one embodiment anti-human POSH antibodies specifically react with the protein encoded by a nucleic acid having SEQ ID NO:2.

The term antibody as used herein is intended to include fragments thereof which are also specifically reactive with one of the subject TGN-associated proteins. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a TGN-associated protein conferred by at least one CDR region of the antibody. In preferred embodiments, the antibodies, the antibody further comprises a label attached thereto and able to be detected, (e.g., the label can be a radioisotope, fluorescent compound, enzyme or enzyme co-factor).

Anti-TGN-associated protein antibodies can be used, e.g., to monitor TGN-associated protein levels, respectively, in an individual, particularly the presence of TGN-associated protein at the plasma membrane for determining whether or not said patient is infected with a virus such as an RNA virus, a retroid virus, and an envelop virus, or allowing determination of the efficacy of a given treatment regimen for an individual afflicted with such a disorder. In addition, TGN-associated protein polypeptides are expected to localize, occasionally, to the released viral particle. Viral particles may be collected and assayed for the presence of a TGN-associated protein. The level of TGN-associated protein may be measured in a variety of sample types such as, for example, cells and/or in bodily fluid, such as in blood samples.

Another application of anti-TGN-associated protein antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a TGN-associated protein, e.g., other orthologs of a particular protein or other paralogs from the same species, can then be detected with antibodies, as, for example, reacting nitrocellulose filters lifted from infected plates with the appropriate anti-TGN-associated protein antibodies. Positive phage detected by this assay can then be isolated from the infected plate. Thus, the presence of TGN-associated protein homologs can be detected and cloned from other animals, as can alternate isoforms (including splice variants) from humans.

6. Transgenic Animals and Uses Thereof

Another aspect of the invention features transgenic non-human animals which express a heterologous TGN-associated protein gene, preferentially a human TGN-associated protein gene of the present invention, and/or which have had one or both copies of the endogenous TGN-associated protein genes disrupted in at least one of the tissue or cell-types of the animal. Accordingly, the invention features an animal model for viral infection. In one embodiment, the transgenic non-human animals is a mammal such as a mouse, rat, rabbit, goat, sheep, dog, cat, cow, or non-human primate. Without being bound to theory, it is proposed that such an animal may be susceptible to infection with envelop viruses, retroid viruses and RNA viruses such as various rhabdoviruses, lentiviruses, and filoviruses. Accordingly, such a transgenic animal may serve as a useful animal model to study the progression of diseases caused by such viruses. Alternatively, such an animal can be useful as a basis to introduce one or more other human transgenes, to create a transgenic animal carrying multiple human genes involved in infection caused by retroid viruses, or RNA viruses, and envelop viruses. Retroid viruses include lentiviruses such as HIV. Other RNA viruses include filoviruses such as Ebola virus. As a result of the introduction of multiple human transgenes, the transgenic animal may become susceptible to certain viral infection and therefore provide an useful animal model to study these viral infection.

The invention additionally features an animal model for the progression of a neurological disorder. In one embodiment, the transgenic non-human animals is a mammal such as a mouse, rat, rabbit, goat, sheep, dog, cat, cow, or non-human primate. Without being bound to theory, it is proposed that the animal model will be susceptible to the onset and progression of a neurological disorder characterized by the aberrant processing of a TGN-associated protein. Accordingly, such an animal model may be useful to study the progression of neurological disorders characterized by such TGN-associated processes. In certain instances, the animal model may be characterized by the presence of plaques composed of beta amyloid peptides, which result from the processing of amyloid beta precursor protein in the TGN.

The invention further features an animal model for the progression of a neoplastic condition. In one embodiment, the transgenic non-human animals is a mammal such as a mouse, rat, rabbit, goat, sheep, dog, cat, cow, or non-human primate. Without being bound to theory, it is proposed that the animal model will be susceptible to the onset and progression of a neoplastic condition, such as cancer, that is characterized by the aberrant processing of a TGN-associated protein. Accordingly, such an animal model may be useful to study the progression of cancers characterized by such TGN-associated processes. In certain instances, the animal model may be characterized by the presence of aberrantly secreted PLD and/or PLD that has been aberrantly activated.

In a preferred embodiment, the transgenic animal carrying human TGN-associated protein gene is useful as a basis to introduce other human genes involved in HIV infection, such as Cyclin T1, CD34, CCR5, and fusin (CRCX4). In a further embodiment, the additional human transgene is a gene involved in a disease or condition that is associated with AIDS (e.g., hypertension, Kaposi's sarcoma, cachexia, etc.) Such an animal may be an useful animal model for studying HIV infection, AIDS and related disease development.

Another aspect of the present invention concerns transgenic animals which are comprised of cells (of that animal) which contain a transgene of the present invention and which preferably (though optionally) express an exogenous TGN-associated protein in one or more cells in the animal. A TGN-associated protein transgene can encode the wild-type form of the protein, or can encode homologs thereof, as well as antisense constructs. Moreover, it may be desirable to express the heterologous TGN-associated protein transgene conditionally such that either the timing or the level of TGN-associated protein gene expression can be regulated. Such conditional expression can be provided using prokaryotic promoter sequences which require prokaryotic proteins to be simultaneous expressed in order to facilitate expression of the TGN-associated protein transgene. Exemplary promoters and the corresponding trans-activating prokaryotic proteins are given in U.S. Pat. No. 4,833,080.

Moreover, transgenic animals exhibiting tissue specific expression can be generated, for example, by inserting a tissue specific regulatory element, such as an enhancer, into the transgene. For example, the endogenous TGN-associated protein gene promoter or a portion thereof can be replaced with another promoter and/or enhancer, e.g., a CMV or a Moloney murine leukemia virus (MLV) promoter and/or enhancer.

Alternatively, non-human transgenic animals that only express HIV transgenes in the brain can be generated using brain specific promoters (e.g., myelin basic protein (MBP) promoter, the neurofilament protein (NF-L) promoter, the gonadotropin-releasing hormone promoter, the vasopressin promoter and the neuron-specific enolase promoter, see So Forss-Petter et al., Neuron, 5, 187, (1990). Such animals can provide a useful in vivo model to evaluate the ability of a potential anti-HIV drug to cross the blood-brain barrier. Other target cells for which specific promoters can be used are, for example, macrophages, T cells and B cells. Other tissue specific promoters are well-known in the art, see e.g., R.Jaenisch, Science, 240, 1468 (1988).

Non-human transgenic animals containing an inducible TGN-associated protein transgene can be generated using inducible regulatory elements (e.g., metallothionein promoter), which are well-known in the art. TGN-associated protein transgene expression can then be initiated in these animals by administering to the animal a compound which induces gene expression (e.g., heavy metals). Another preferred inducible system comprises a tetracycline-inducible transcriptional activator (U.S. Pat. No. 5,654,168 issued Aug. 5, 1997 to Bujard and Gossen and U.S. Pat. No. 5,650,298 issued Jul. 22, 1997 to Bujard et al.).

In general, transgenic animal lines can be obtained by generating transgenic animals having incorporated into their genome at least one transgene, selecting at least one founder from these animals and breeding the founder or founders to establish at least one line of transgenic animals having the selected transgene incorporated into their genome.

Animals for obtaining eggs or other nucleated cells (e.g., embryonic stem cells) for generating transgenic animals can be obtained from standard commercial sources such as Charles River Laboratories (Wilmington, Mass.), Taconic (Germantown, N.Y.), Harlan Sprague Dawley (Indianapolis, Ind.).

Eggs can be obtained from suitable animals, e.g., by flushing from the oviduct or using techniques described in U.S. Pat. No. 5,489,742 issued Feb. 6, 1996 to Hammer and Taurog; U.S. Pat. No. 5,625,125 issued on Apr. 29, 1997 to Bennett et al.; Gordon et al., 1980, Proc. Natl. Acad. Sci. USA 77:7380-7384; Gordon & Ruddle, 1981, Science 214: 1244-1246; U.S. Pat. No. 4,873,191 to T. E. Wagner and P. C. Hoppe; U.S. Pat. No. 5,604,131; Armstrong, et al. (1988) J. of Reproduction, 39:511 or PCT application No. PCT/FR93/00598 (WO 94/00568) by Mehtali et al. Preferably, the female is subjected to hormonal conditions effective to promote superovulation prior to obtaining the eggs.

Many techniques can be used to introduce DNA into an egg or other nucleated cell, including in vitro fertilization using sperm as a carrier of exogenous DNA (“sperm-mediated gene transfer”, e.g., Lavitrano et al., 1989, Cell 57: 717-723), microinjection, gene targeting (Thompson et al., 1989, Cell 56: 313-321), electroporation (Lo, 1983, Mol. Cell. Biol. 3: 1803-1814), transfection, or retrovirus mediated gene transfer (Van der Putten et al., 1985, Proc. Natl. Acad. Sci. USA 82: 6148-6152). For a review of such techniques, see Gordon (1989), Transgenic Animals, Intl. Rev. Cytol. 115:171-229.

Except for sperm-mediated gene transfer, eggs should be fertilized in conjunction with (before, during or after) other transgene transfer techniques. A preferred method for fertilizing eggs is by breeding the female with a fertile male. However, eggs can also be fertilized by in vitro fertilization techniques.

Fertilized, transgene containing eggs can than be transferred to pseudopregnant animals, also termed “foster mother animals”, using suitable techniques. Pseudopregnant animals can be obtained, for example, by placing 40-80 day old female animals, which are more than 8 weeks of age, in cages with infertile males, e.g., vasectomized males. The next morning females are checked for vaginal plugs. Females who have mated with vasectomized males are held aside until the time of transfer.

Recipient females can be synchronized, e.g., using GNRH agonist (GnRH-a): des-gly10, (D-Ala6)-LH-RH Ethylamide, SigmaChemical Co.,St. Louis, Mo. Alternatively, a unilateral pregnancy can be achieved by a brief surgical procedure involving the “peeling” away of the bursa membrane on the left uterine horn. Injected embryos can then be transferred to the left uterine horn via the infundibulum. Potential transgenic founders can typically be identified immediately at birth from the endogenous litter mates. For generating transgenic animals from embryonic stem cells, see e.g., Teratocarcinomas and embryonic stem cells, a practical approach, ed. E. J. Robertson, (IRL Press 1987) or in Potter et al Proc. Natl. Acad. Sci. USA 81, 7161 (1984), the teachings of which are incorporated herein by reference.

Founders that express the gene can then bred to establish a transgenic line. Accordingly, founder animals can be bred, inbred, crossbred or outbred to produce colonies of animals of the present invention. Animals comprising multiple transgenes can be generated by crossing different founder animals (e.g., an HIV transgenic animal and a transgenic animal, which expresses human CD4), as well as by introducing multiple transgenes into an egg or embryonic cell as described above. Furthermore, embryos from A-transgenic animals can be stored as frozen embryos, which are thawed and implanted into pseudo-pregnant animals when needed (See e.g., Hirabayashi et al. (1997) Exp Anim 46: 111 and Anzai (1994) Jikken Dobutsu 43: 247).

The present invention provides for transgenic animals that carry the transgene in all their cells, as well as animals that carry the transgene in some, but not all cells, i.e., mosaic animals. The transgene can be integrated as a single transgene or in tandem, e.g., head to head tandems, or head to tail or tail to tail or as multiple copies.

The successful expression of the transgene can be detected by any of several means well known to those skilled in the art. Non-limiting examples include Northern blot, in situ hybridization of mRNA analysis, Western blot analysis, immunohistochemistry, and FACS analysis of protein expression.

In a further aspect, the invention features non-human animal cells containing a TGN-associated protein transgene, preferentially a human TGN-associated protein transgene. For example, the animal cell (e.g., somatic cell or germ cell (i.e. egg or sperm)) can be obtained from the transgenic animal. Transgenic somatic cells or cell lines can be used, for example, in drug screening assays. Transgenic germ cells, on the other hand, can be used in generating transgenic progeny, as described above.

The invention further provides methods for identifying (screening) or for determining the safety and/or efficacy of virus therapeutics, i.e. compounds which are useful for treating and/or preventing the development of diseases or conditions, which are caused by, or contributed to by viral infection (e.g., AIDS). In addition the assays are useful for further improving known anti-viral compounds, e.g, by modifying their structure to increase their stability and/or activity and/or toxicity.

The transgenic animals can be used in in vivo assays to identify viral therapeutics. For example, the animals can be used in assays to identify compounds which reduce or inhibit any phase of the viral life cycle, e.g., expression of one or more viral genes, activity of one or more viral proteins, glycosylation of one or more viral proteins, processing of one or more viral proteins, viral replication, assembly of virions, and/or budding of infectious virions.

In an exemplary embodiment, the assay comprises administering a test compound to a transgenic animal of the invention infected with a virus including RNA viruses, DNA viruses, retroidvirus and/or envelop viruses, and comparing a phenotypic change in the animal relative to a transgenic animal which has not received the test compound. For example, where the animal is infected with HIV, the phenotypic change can be the amelioration in an AIDS related complex (ARC), cataracts, inflammatory lesions in the central nervous system (CNV), a mild kidney sclerotic lesion, or a skin lesion, such as psoratic dermatitis, hyperkerstotic lesions, Kaposi's sarcoma or cachexia. The effect of a compound on inhibition of Kaposi's sarcoma can be determined, as described, e.g., in PCT/US97/11202 (WO97/49373) by Gallo et al. These and other HIV related symptoms or phenotypes are further described in Leonard et al. (1988) Science 242:1665.

In another embodiment, the phenotypic change is release/budding of virus particles. In yet another embodiment, the phenotypic change is the number of CD4+ T cells or the ratio of CD4+ T cells versus CD8+ T cells. In HIV infected humans as well as in HIV transgenic mice, analysis of lymph nodes indicate that the number of CD4+ T cells decreases and the number of CD8+ T cells increases. Numbers of CD4+ and CD8+ T cells can be determined, for example, by indirect immunofluorescence and flow cytometry, as described, e.g., in Santoro et al., supra.

Alternatively, a phenotypic change, e.g., a change in the expression level of an HIV gene can be monitored. The HIV RNA can be selected from the group consisting of gag mRNA, gag-pro-pol mRNA, vif mRNA, vpr mRNA, tat mRNA, rev mRNA, vpu/env mRNA, nef mRNA, and vpx mRNA. The HIV protein can be selected from the group consisting of Pr55 Gag and fragments thereof (p17 MA, p24 CA, p7 NC, p1, p9, p6, and p2), Pr160 Gag-Pro-Pol, and fragments thereof (p10 PR, p51 RT, p66 RT, p32 IN), p23 Vif, p15 Vpr, p14 Tat, p19 Rev, p16 Vpu, gPr 160 Env or fragments thereof (gp120 SU and gp41TM), p27 Nef, and p14 Vpx. The level of any of these mRNAs or proteins can be determined in cells from a tissue sample, such as a skin biopsy, as described in, e.g., PCT/US97/11202 (W097/49373) by Gallo et al. Quantitation of HIV mRNA and protein is further described elsewhere herein and also in, e.g., Dickie et al. (1996) AIDS Res. Human Retroviruses 12:1103. In a preferred embodiment, the level of gp120 on the surface of PBMC is determined. This can be done, as described in the examples, e.g., by immunofluorescence on PBMC obtained from the animals.

A further phenotypic change is the production level or rate of viral particles in the serum and/or tissue of the animal. This can be determined, e.g., by determining reverse transcriptase (RT activity) or viral load as described elsewhere herein as well as in PCT/US97/11202 (WO97/49373) by Gallo et al., such as by determining p24 antigen.

Yet another phenotypic change, which can indicate HIV infection or AIDS progression is the production of inflammatory cytokines such as IL-6, IL-8 and TNF-.alpha.; thus, efficacy of a compound as an anti-HIV therapeutic can be assessed by ELISA tests for the reduction of serum levels of any or all of these cytokines.

A vaccine can be tested by administering a test antigen to a transgenic animal of the invention. The animal can optionally be boosted with the same or a different antigen. Such animal is then infected with a virus such as HIV. The production of viral particles or expression of viral proteins is then measured at various times following the administration of the test vaccine. A decrease in the amount of viral particles produced or viral expression will indicate that the test vaccine is efficient in reducing or inhibiting viral production and/or expression. The amount of antibody produced by the animal in response to the vaccine antigen can also be determined according to methods known in the art and provides a relative indication of the immunogenicity of the particular antigen.

Cells from the transgenic animals of the invention can be established in culture and immortalized to establish cell lines. For example, immortalized cell lines can be established from the livers of transgenic rats, as described in Bulera et al. (1997) Hepatology 25: 1192. Cell lines from other types of cells can be established according to methods known in the art.”

In one cell-based assay, cells expressing a TGN-associated protein transgene can be infected with a virus of interest and incubated in the presence a test compound or a control compound. The production of viral particles is then compared. This assay system thus provides a means of identifying molecular antagonists which, for example, function by interfering with viral release/budding.

Cell based assays can also be used to identify compounds which modulate expression of a viral gene, modulate translation of a viral mRNA, or which modulate the stability of a viral mRNA or protein. Accordingly, a cell which is infected with a virus of interest can be incubated with a test compound and the amount of the viral protein produced in the cell medium can be measured and compared to that produced from a cell which has not been contacted with the test compound. The specificity of the compound for regulating the expression of the particular virus gene can be confirmed by various control analyses, e.g., measuring the expression of one or more control genes. This type of cellular assay can be particularly useful for determining the efficacy of antisense molecules or ribozymes.

7. RNA Interference, Ribozymes, Antisense and DNA Enzyme

In certain aspects, the invention relates to RNAi, ribozyme, antisense and other nucleic acid-related methods and compositions for manipulating (typically decreasing) a TGN-associated protein activity. Exemplary RNAi and ribozyme molecules may comprise a sequence as shown in any of SEQ ID NOS: 15, 16, 18, 19, 21, 22, 24 and 25.

Certain embodiments of the invention make use of materials and methods for effecting knockdown of one or more TGN-associated protein genes by means of RNA interference (RNAi), using an RNAi construct. RNAi is a process of sequence-specific post-transcriptional gene repression which can occur in eukaryotic cells. In general, this process involves degradation of an mRNA of a particular sequence induced by duplex containing an antisense RNA that hybridizes to the target sequence. An RNAi construct may, for example, be a double stranded RNA (dsRNA), a DNA:RNA hybrid or a hairpin RNA comprising a short duplex region. In general, the sense portion of the duplex is amenable to modifications, while the antisense portion should generally be unmondified RNA or mostly unmodified RNA. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Furthermore, Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′, 5′ oligoadenylate synthetase (2′, 5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8). RNAi has been shown to be effective in reducing or eliminating the expression of a POSH gene in a number of different organisms including Caenorhabditiis elegans (see e.g., Fire et al. (1998) Nature 391: 806-11), mouse eggs and embryos (Wianny et al. (2000) Nature Cell Biol 2: 70-5; Svoboda et al. (2000) Development 127: 4147-56), and cultured RAT-1 fibroblasts (Bahramina et al. (1999) Mol Cell Biol 19: 274-83), and appears to be an anciently evolved pathway available in eukaryotic plants and animals (Sharp (2001) Genes Dev. 15: 485-90). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany). Synthetic oligonucleotides are preferably deprotected and gel-purified using methods known in the art (see e.g., Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a TGN-associated protein nucleic acid, such as, for example, a nucleic acid that hybridizes, under stringent and/or physiological conditions, to any of SEQ ID NOS: 1, 3, 4, 6, 8, 10, 37-74, 98-115, and complements thereof.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g., Jaeger et al. (1989) Proc. Natl. Acad. Sci. USA 86:7706 (1989); and Turner et al. (1988) Annu. Rev. Biophys. Biophys. Chem. 17:167) . The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerheadribozyme compositions of the invention.

The dsRNA oligonucleotides may be introduced into the cell by transfection with an heterologous target gene using carrier compositions such as liposomes, which are known in the art—e.g., Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al. (1998) J Cell Biol 141: 863-74). The effectiveness of the RNAi may be assessed by any of a number of assays following introduction of the dsRNAs. These include Western blot analysis using antibodies which recognize the TGN-associated protein gene product following sufficient time for turnover of the endogenous pool after new protein synthesis is repressed, reverse transcriptase polymerase chain reaction and Northern blot analysis to determine the level of existing TGN-associated protein target mRNA.

Further compositions, methods and applications of RNAi technology are provided in U.S. patent application Nos. 6,278,039, 5,723,750 and 5,244,805, which are incorporated herein by reference.

Ribozyme molecules designed to catalytically cleave TGN-associated protein mRNA transcripts can also be used to prevent translation of subject TGN-associated protein mRNAs and/or expression of TGN-associated protein (see, e.g., PCT International Publication WO90/11364, published Oct. 4, 1990; Sarver et al. (1990) Science 247:1222-1225 and U.S. Pat. No. 5,093,246). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a TGN-associated protein mRNA, and the well known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach ((1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J). In particular, RNA polymerase III-mediated expression of tRNA fusion ribozymes are well known in the art ( see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, l9 or20 contiguousnucleotides in length of a TGN-associated protein mRNA, such as an mRNA of a sequence represented in any of SEQ ID NOS: 1, 3, 4, 6, 8, 10, 37-74, or 98-115. In addition, ribozymes possess highly specific endoribonuclease activity, which autocatalytically cleaves the target sense mRNA. The present invention extends to ribozymes which hybridize to a sense mRNA encoding a TGN-associated protein gene such as a therapeutic drug target candidate gene, thereby hybridising to the sense mRNA and cleaving it, such that it is no longer capable of being translated to synthesize a functional polypeptide product.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al. (1984) Science 224:574-578; Zaug, et al. (1986) Science 231:470-475; Zaug, et al. (1986) Nature 324:429-433; published International patent application No. WO88/04300 by University Patents Inc.; Been, et al. (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or RNAi are substantially the same sequence of at least 5 and preferably 6, 7, 8, 9, 10, 11, 12, 13, 14, 15, 16, 17, 18, 19 or 20 or more contiguous nucleotides of a TGN-associated protein nucleic acid, such as a nucleic acid of any of SEQ ID NOS: 1, 3, 4, 6, 8, 10, 37-74, or 98-115. In a long target RNA chain, significant numbers of target sites are not accessible to the ribozyme because they are hidden within secondary or tertiary structures (Birikh et al. (1997) Eur J Biochem 245: 1-16). To overcome the problem of target RNA accessibility, computer generated predictions of secondary structure are typically used to identify targets that are most likely to be single-stranded or have an “open” configuration (see Jaeger et al. (1989) Methods Enzymol 183: 281-306). Other approaches utilize a systematic approach to predicting secondary structure which involves assessing a huge number of candidate hybridizing oligonucleotides molecules (see Milner et al. (1997) Nat Biotechnol 15: 537-41; and Patzel and Sczakiel (1998) Nat Biotechnol 16: 64-8). Additionally, U.S. Pat. No. 6,251,588, the contents of which are hereby incorporated herein, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the RNAi oligonucleotides and ribozymes of the invention.

A further aspect of the invention relates to the use of the isolated “antisense” nucleic acids to inhibit expression, e.g., by inhibiting transcription and/or translation of a subject TGN-associated protein nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a TGN-associated polypeptide. Alternatively, the antisense construct is an oligonucleotide probe, which is generated ex vivo and which, when introduced into the cell causes inhibition of expression by hybridizing with the mRNA and/or genomic sequences of a TGN-associated protein nucleic acid. Such oligonucleotide probes are preferably modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659- 2668.

With respect to antisense DNA, oligodeoxyribonucleotides derived from the translation initiation site, e.g., between the −10 and +10 regions of the TGN-associated protein gene, are preferred. Antisense approaches involve the design of oligonucleotides (either DNA or RNA) that are complementary to mRNA encoding the TGN-associated polypeptide. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. 1994. Nature 372:333). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less that about 100 and more preferably less than about 50, 25, 17 or 10 nucleotides in length.

It is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Results obtained using the antisense oligonucleotide may be compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958- 976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

While antisense nucleotides complementary to the coding region of a TGN-associated protein mRNA sequence can be used, those complementary to the transcribed untranslated region may also be used.

In certain instances, it may be difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells will result in the transcription of sufficient amounts of single stranded RNAs that will form complementary base pairs with the endogenous potential drug target transcripts and thereby prevent translation. For example, a vector can be introduced such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct, which can be introduced directly into the tissue site.

Alternatively, TGN-associated protein gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C. 1991, Anticancer Drug Des., 6(6):569-84; Helene, C., et al., 1992, Ann. N.Y. Acad. Sci., 660:27-36; and Maher, L. J., 1992, Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine- rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential TGN-associated protein sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of a TGN-associated protein gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined in detail above. Additionally, one of skill in the art will recognize that, like antisense oligonucleotide, DNA enzymes can be optionally modified to improve stability and improve resistance to degradation.

Antisense RNA and DNA, ribozyme, RNAi and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

8. Drug Screening Assays

In certain aspects, the present invention also provides assays for identifying therapeutic agents that interfere with the trafficking and/or assembly of protein assemblies in the trans Golgi network, including proteins associated with microbial-related disorders, cancers, or neurological disorders. In certain embodiments, agents of the invention are antiviral agents, optionally interfering with viral maturation, and preferably where the virus is a retroid virus, an RNA virus and an envelop virus. In certain preferred embodiments, an antiviral agent interferes with the ubiquitin ligase catalytic activity of TGN-associated protein (e.g., TGN-associated protein auto-ubiquitination or transfer to a target protein). In certain preferred embodiments, an antiviral agent interferes with the interaction between POSH and a POSH-AP polypeptide, for example an antiviral agent may disrupt or render irreversible the interaction between a POSH polypeptide and POSH-AP polypeptide such as another POSH polypeptide (as in the case of a POSH dimer, a heterodimer of two different POSH polypeptides, homomultimers and heteromultimers); an UNC84, an MSTP28, a HERPUD1, a Cbl-b, a GOCAP1, a PTPN12, a GOSR2, a RALA, a SIAH1, an SMN1, an SMN2, a SYNE1, a TTC3, a VCY2IP1, a PKA, an EIF3S3; a GTPase (eg. Rac, Rac1, Rho, Ras); an E2 enzyme and ubiquitin, or optionally, a cullin; a clathrin; AP-1; AP-2; an HSP70; an HSP90, Brca1, Bard1, Nef, PAK1, PAK2, PAK family, Vav, Cdc42, PI3K (e.g., p85 or p110), Nedd4, src (src family), a Gag, particularly an HIV Gag (e.g., p160), Tsg101, VASP, RNB6, WASP, N-WASP and KIAA0674, Similar to Spred-2, as well as, in certain embodiments, proteins known to be associated with clathrin-coated vesicles and or proteins involved in the protein sorting pathway. In further embodiments, agents of the invention are anti-apoptotic agents, optionally interfering with JNK and/or NF-κB signaling, or PLD activity and/or secretion. In additional embodiments of the invention, agents of the invention are drugs that inhibit the onset or progression of a neurological disorder, optionally interfering with the TGN-associated process of aberrant protein processing (e.g., the processing of amyloid beta precursor protein). In yet additional embodiments, agents of the invention interfere with the signaling of a GTPase, such as Rac, Ras, or RalA, optionally disrupting the interaction between a TGN-associated polypeptide and a Rac protein. In certain embodiments, agents of the invention modulate the ubiquitin ligase activity of a TGN-associated protein, such as POSH or Cbl-b, and may be used to treat certain diseases related to ubiquitin ligase activity, such as certain cancers and neurological disorders.

In certain embodiments, the invention provides assays to identify, optimize or otherwise assess agents that increase or decrease a ubiquitin-related activity of a TGN-associated protein. Ubiquitin-related activities of TGN-associated proteins may include, for example, the self-ubiquitination activity of a TGN-associated protein, generally involving the transfer of ubiquitin from an E2 enzyme to the TGN-associated protein, and the ubiquitination of a target protein, generally involving the transfer of a ubiquitin from a TGN-associated protein to the target protein. In certain embodiments, a TGN-associated protein activity is mediated, at least in part, by a RING domain. In further embodiments, a TGN-associated protein activity is mediated, at least in part, by a TGN-localization domain.

In certain embodiments, an assay comprises forming a mixture comprising a TGN-associated protein that is an E3 polypeptide, an E2 polypeptide and a source of ubiquitin (which may be the E2 polypeptide pre-complexed with ubiquitin). Optionally the mixture comprises an E1 polypeptide and optionally the mixture comprises a target polypeptide. Additional components of the mixture may be selected to provide conditions consistent with the ubiquitination of the TGN-associated E3 polypeptide. One or more of a variety of parameters may be detected, such as TGN-associated protein-ubiquitin conjugates, E2-ubiquitin thioesters, free ubiquitin and target polypeptide-ubiquitin complexes. The term “detect” is used herein to include a determination of the presence or absence of the subject of detection (e.g., POSH-ubiqutin, Cbl-b-ubiquitin, E2-ubiquitin, etc.), a quantitative measure of the amount of the subject of detection, or a mathematical calculation of the presence, absence or amount of the subject of detection, based on the detection of other parameters. The term “detect” includes the situation wherein the subject of detection is determined to be absent or below the level of sensitivity. Detection may comprise detection of a label (e.g., fluorescent label, radioisotope label, and other described below), resolution and identification by size (e.g., SDS-PAGE, mass spectroscopy), purification and detection, and other methods that, in view of this specification, will be available to one of skill in the art. For instance, radioisotope labeling may be measured by scintillation counting, or by densitometry after exposure to a photographic emulsion, or by using a device such as a Phosphorimager. Likewise, densitometry may be used to measure bound ubiquitin following a reaction with an enzyme label substrate that produces an opaque product when an enzyme label is used. In a preferred embodiment, an assay comprises detecting the TGN-associated protein-ubiquitin conjugate.

In certain embodiments, an assay comprises forming a mixture comprising a TGN-associated protein, a target polypeptide and a source of ubiquitin (which may be the TGN-associated polypeptide pre-complexed with ubiquitin). Optionally the mixture comprises an E1 and/or E2 polypeptide and optionally the mixture comprises an E2-ubiquitin thioester. Additional components of the mixture may be selected to provide conditions consistent with the ubiquitination of the target polypeptide. One or more of a variety of parameters may be detected, such as TGN-associated protein-ubiquitin conjugates and target polypeptide-ubiquitin conjugates. In a preferred embodiment, an assay comprises detecting the target polypeptide-ubiquitin conjugate. In another preferred embodiment, an assay comprises detecting the TGN-associated protein-ubiquitin conjugate.

An assay described above may be used in a screening assay to identify agents that modulate a ubiquitin-related activity of a TGN-associated polypeptide. A screening assay will generally involve adding a test agent to one of the above assays, or any other assay designed to assess a ubiquitin-related activity of a TGN-associated protein. The parameter(s) detected in a screening assay may be compared to a suitable reference. A suitable reference may be an assay run previously, in parallel or later that omits the test agent. A suitable reference may also be an average of previous measurements in the absence of the test agent. In general the components of a screening assay mixture may be added in any order consistent with the overall activity to be assessed, but certain variations may be preferred. For example, in certain embodiments, it may be desirable to pre-incubate the test agent and the E3 (e.g., the TGN-associated protein), followed by removing the test agent and addition of other components to complete the assay. In this manner, the effects of the agent solely on the TGN-associated protein may be assessed. In certain preferred embodiments, a screening assay for an antiviral agent employs a target polypeptide comprising an L domain, and preferably an HIV L domain.

In certain embodiments, an assay is performed in a high-throughput format. For example, one of the components of a mixture may be affixed to a solid substrate and one or more of the other components is labeled. For example, the TGN-associated protein may be affixed to a surface, such as a 96-well plate, and the ubiquitin is in solution and labeled. An E2 and E1 are also in solution, and the TGN-associated protein-ubiquitin conjugate formation may be measured by washing the solid surface to remove uncomplexed labeled ubiquitin and detecting the ubiquitin that remains bound. Other variations may be used. For example, the amount of ubiquitin in solution may be detected. In certain embodiments, the formation of ubiquitin complexes may be measured by an interactive technique, such as FRET, wherein a ubiquitin is labeled with a first label and the desired complex partner (e.g., TGN-associated protein or target polypeptide) is labeled with a second label, wherein the first and second label interact when they come into close proximity to produce an altered signal. In FRET, the first and second labels are fluorophores. FRET is described in greater detail below. The formation of polyubiquitin complexes may be performed by mixing two or more pools of differentially labeled ubiquitin that interact upon formation of a polyubiqutin (see, e.g., US Patent Publication 20020042083). High-throughput may be achieved by performing an interactive assay, such as FRET, in solution as well. In addition, if a polypeptide in the mixture, such as the TGN-associated protein or target polypeptide, is readily purifiable (e.g., with a specific antibody or via a tag such as biotin, FLAG, polyhistidine, etc.), the reaction may be performed in solution and the tagged polypeptide rapidly isolated, along with any polypeptides, such as ubiquitin, that are associated with the tagged polypeptide. Proteins may also be resolved by SDS-PAGE for detection.

In certain embodiments, the ubiquitin is labeled, either directly or indirectly. This typically allows for easy and rapid detection and measurement of ligated ubiquitin, making the assay useful for high-throughput screening applications. As descrived above, certain embodiments may employ one or more tagged or labeled proteins. A “tag” is meant to include moieties that facilitate rapid isolation of the tagged polypeptide. A tag may be used to facilitate attachment of a polypeptide to a surface. A “label” is meant to include moieties that facilitate rapid detection of the labeled polypeptide. Certain moieties may be used both as a label and a tag (e.g., epitope tags that are readily purified and detected with a well-characterized antibody). Biotinylation of polypeptides is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known.

An “E1” is a ubiquitin activating enzyme. In a preferred embodiment, E1 is capable of transferring ubiquitin to an E2. In a preferred embodiment, E1 forms a high energy thiolester bond with ubiquitin, thereby “activating” the ubiquitin. An “E2” is a ubiquitin carrier enzyme (also known as a ubiquitin conjugating enzyme). In a preferred embodiment, ubiquitin is transferred from E1 to E2. In a preferred embodiment, the transfer results in a thiolester bond formed between E2 and ubiquitin. In a preferred embodiment, E2 is capable of transferring ubiquitin to a TGN-associated protein.

In an alternative embodiment, a TGN-associated protein, E2 or target polypeptide is bound to a bead, optionally with the assistance of a tag. Following ligation, the beads may be separated from the unbound ubiquitin and the bound ubiquitin measured. In a preferred embodiment, TGN-associated protein is bound to beads and the composition used includes labeled ubiquitin. In this embodiment, the beads with bound ubiquitin may be separated using a fluorescence-activated cell sorting (FACS) machine. Methods for such use are described in U.S. patent application Ser. No. 09/047,119, which is hereby incorporated in its entirety. The amount of bound ubiquitin can then be measured.

In a screening assay, the effect of a test agent may be assessed by, for example, assessing the effect of the test agent on kinetics, steady-state and/or endpoint of the reaction.

The components of the various assay mixtures provided herein may be combined in varying amounts. In a preferred embodiment, ubiquitin (or E2 complexed ubiquitin) is combined at a final concentration of from 5 to 200 ng per 100 microliter reaction solution. Optionally E1 is used at a final concentration of from 1 to 50 ng per 100 microliter reaction solution. Optionally E2 is combined at a final concentration of 10 to 100 ng per 100 microliter reaction solution, more preferably 10-50 ng per 100 microliter reaction solution. In a preferred embodiment, TGN-associated protein is combined at a final concentration of from 1 ng to 500 ng per 100 microliter reaction solution.

Generally, an assay mixture is prepared so as to favor ubiquitin ligase activity and/or ubiquitination acitivty. Generally, this will be physiological conditions, such as 50-200 mM salt (e.g., NaCl, KCl), pH of between 5 and 9, and preferably between 6 and 8. Such conditions may be optimized through trial and error. Incubations may be performed at any temperature which facilitates optimal activity, typically between 4 and 40 degrees C. Incubation periods are selected for optimum activity, but may also be optimized to facilitate rapid high through put screening. Typically between 0.5 and 1.5 hours will be sufficient. A variety of other reagents may be included in the compositions. These include reagents like salts, solvents, buffers, neutral proteins, e.g., albumin, detergents, etc. which may be used to facilitate optimal ubiquitination enzyme activity and/or reduce non-specific or background interactions. Also reagents that otherwise improve the efficiency of the assay, such as protease inhibitors, nuclease inhibitors, anti-microbial agents, etc., may be used. The compositions will also preferably include adenosine tri-phosphate (ATP). The mixture of components may be added in any order that promotes ubiquitin ligase activity or optimizes identification of candidate modulator effects. In a preferred embodiment, ubiquitin is provided in a reaction buffer solution, followed by addition of the ubiquitination enzymes. In an alternate preferred embodiment, ubiquitin is provided in a reaction buffer solution, a candidate modulator is then added, followed by addition of the ubiquitination enzymes.

In general, a test agent that decreases a TGN-associated protein ubiquitin-related activity may be used to inhibit TGN-associated protein function in vivo, while a test agent that increases a TGN-associated protein ubiquitin-related activity may be used to stimulate TGN-associated protein function in vivo. Test agent may be modified for use in vivo, e.g., by addition of a hydrophobic moiety, such as an ester.

Certain embodiments of the invention relate to assays for identifying agents that bind to a TGN-associated protein, optionally a particular domain of TGN-associated protein such as a TGN-localization domain, or an SH3 or RING domain. In certain embodiments, a TGN-associated protein is a POSH polypeptide that is a polypeptide comprising the fourth SH3 domain of HPOSH (SEQ ID NO: 30). A wide variety of assays may be used for this purpose, including labeled in vitro protein-protein binding assays, electrophoretic mobility shift assays, immunoassays for protein binding, and the like. The purified protein may also be used for determination of three-dimensional crystal structure, which can be used for modeling intermolecular interactions and design of test agents. In one embodiment, an assay detects agents which inhibit interaction of one or more subject POSH polypeptides with a POSH-AP. In another embodiment, the assay detects agents which modulate the intrinsic biological activity of a TGN-associated protein or TGN-associated protein complex, such as an enzymatic activity, binding to other cellular components, cellular compartmentalization, and the like.

In one aspect, the invention provides methods and compositions for the identification of compositions that interfere with the function of TGN-associated protein. Given the role of TGN-associated proteins in viral production, cellular transformation, cell proliferation, and aberrant protein processing associated with neurological disorders, compositions that perturb the formation or stability of the protein-protein interactions between TGN-associated proteins and the proteins that they interact with are candidate pharmaceuticals for the treatment of viral infections.

While not wishing to be bound to mechanism, it is postulated that TGN-associated protein promote the assembly of protein complexes that are important in release of virions and other biological processes. Complexes of the invention may include a combination of a POSH-pathway polypeptide (e.g., PLD), a POSH polypeptide and one or more of the following POSH-APs: a POSH-AP; a POSH polypeptide (as in the case of a POSH dimer, a heterodimer of two different POSH, homomultimers and heteromultimers); Vpu; Cbl-b; a PKA; an UNC48B; an MSTP028; a HERPUD1; a GOCAP1; a PTPN12; an EIF3S3; an SRA1; a GOSR2; a RALA; a SIAH1; an SMN1; an SMN2; a SYNE1; a TTC3; a VCY2IP1; a GTPase (eg. Rac, Rac1, Rho, Ras); an E2 enzyme; ubiquitin, or optionally, a cullin; a clathrin; AP-1; AP-2; an HSP70; an HSP90, Brca1, Bard1, Nef, PAK1, PAK2, PAK family, Vav, Cdc42, PI3K (e.g., p85 or p110), Nedd4, src (src family), Tsg101, VASP, RNB6, WASP, N-WASP, a Gag, particularly an HIV Gag (e.g., p160); and KIAA0674, Similar to Spred-2, as well as, in certain embodiments, proteins known to be associated with clathrin-coated vesicles and or proteins involved in the protein sorting pathway.

The type of complex formed by a TGN-associated polypeptide will depend upon the domains present in the protein. While not intended to be limiting, exemplary domains of potential interacting proteins are provided below. A TGN localization domain is expected to localize a protein to the TGN. A RING domain is expected to interact with cullins, E2 enzymes, AP-1, AP-2, and/or a substrate for ubiquitination (e.g., in some instances, a protein comprising a Gag L domain or a Gag polypeptide such as Gag-Pol, such as HIV p160). An SH3 domain may interact with Gag L domains and other proteins having the sequence motif P(T/S)AP (SEQ ID NO: 169), RXXP(T/S)AP (SEQ ID NO: 173), PXXDY, PXXP, PPXY, RXXPXXP, or PXXPXR such as, for example, an HIV Gag sequence such as RQGPKEPFR (SEQ ID NO: 172), PFRDY (SEQ ID NO: 170), PTAP (SEQ ID NO: 174) and RPEPTAP (SEQ ID NO: 164).

In a preferred assay for an antiviral or antiapoptotic agent, the test agent is assessed for its ability to disrupt or inhibit the formation of a complex of a TGN-associated polypeptide and a Rac polypeptide, particularly a human Rac polypeptide, such as Rac1.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even a TGN-associated protein-mediated membrane reorganization or vesicle formation activity, may be generated in many different forms, and include assays based on cell-free systems, e.g., purified proteins or cell lysates, as well as cell-based assays which utilize intact cells. Simple binding assays can also be used to detect agents which bind to a TGN-associated protein. Such binding assays may also identify agents that act by disrupting the interaction between a TGN-associated protein and an interacting protein, or the binding of a TGN-associated protein or complex to a substrate. Agents to be tested can be produced, for example, by bacteria, yeast or other organisms (e.g., natural products), produced chemically (e.g., small molecules, including peptidomimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted TGN-associated protein complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in TGN-associated protein complex formation are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure TGN-associated protein complex assembly and/or disassembly.

Assaying TGN-associated protein complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of the TGN-associated protein complex. In an exemplary binding assay, the compound of interest is contacted with a mixture comprising a TGN-associated protein and at least one interacting polypeptide. Detection and quantification of TGN-associated protein complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the TGN-associated proteins and a substrate polypeptide may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction

Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-TGN-associated protein fusion proteins can be adsorbed onto glutathione sepharose beads (Sigma Chemical, St. Louis, Mo.) or glutathione derivatized microtitre plates, which are then combined with a potential interacting protein, e.g., an 35S-labeled polypeptide, and the test compound and incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In a further embodiment, agents that bind to a TGN-associated protein may be identified by using an immobilized TGN-associated protein. In an illustrative embodiment, a fusion protein can be provided which adds a domain that permits the protein to be bound to an insoluble matrix. For example, GST-TGN-associated protein fusion proteins can be adsorbed onto glutathione sepharose beads (Sigma Chemical, St. Louis, Mo.) or glutathione derivatized microtitre plates, which are then combined with a potential labeled binding agent and incubated under conditions conducive to binding. Following incubation, the beads are washed to remove any unbound agent, and the matrix bead-bound label determined directly, or in the supernatant after the bound agent is dissociated.

In yet another embodiment, the TGN-associated protein and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., a TGN-associated protein of sufficient length to bind to a potential interacting protein. The second hybrid protein encodes a transcriptional activation domain fused in frame to a gene encoding a “fish” protein, e.g., a potential interacting protein of sufficient length to interact with the TGN-associated protein portion of the bait fusion protein. If the bait and fish proteins are able to interact, e.g., form a TGN-associated protein complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector. Interaction trap assays may also be performed in mammalian and bacterial cell types.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a TGN-associated protein sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADRI proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, lcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative or other mutants of a TGN-associated protein can be used.

Continuing with the illustrated example, the TGN-associated protein-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a TGN-associated protein complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations including a TGN-associated protein and one or more interacting polypeptides.

In still further embodiments of the present assay, the TGN-associated protein complex is generated in whole cells, taking advantage of cell culture techniques to support the subject assay. For example, as described below, the TGN-associated protein complex can be constituted in a eukaryotic cell culture system, including mammalian and yeast cells. Often it will be desirable to express one or more viral proteins (eg. Gag or Env or Vpu) in such a cell along with a subject TGN-associated protein. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the TGN-associated protein complex can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In many embodiments, a cell is manipulated after incubation with a candidate agent and assayed for a TGN-associated protein activity. In certain embodiments a TGN-associated protein activity is represented by production of virus like particles, by secretion of a protein involved in the pathogenesis of a neoplastic condition, or the improper processing of a protein that is associated with a degenerative neurological disorder. As demonstrated herein, an agent that disrupts TGN-associated protein activity can cause a decrease in the production of virus like particles, secreted proteins, or improperly processed proteins. Other bioassays for TGN-associated protein activities may include apoptosis assays (e.g., cell survival assays, apoptosis reporter gene assays, etc.) and NF-kB nuclear localization assays (see e.g., Tapon et al. (1998) EMBO J. 17: 1395-1404). One apoptosis assay that may be used to assess TGN-associated protein activity is the TUNEL assay, which is used to detect the presence of apoptotic cell death. In the TUNEL assay, the enzyme terminal deoxynucleotidyl transferase labels 3′-OH DNA ends (which are generated during apoptosis) with biotinylated nucleotides. The biotinylated nucleotides are then detected by immunoperoxidase staining. Another apoptosis assay that may be used to assess TGN-associated protein activity is the caspase assay, in which caspase activity is measured using a blue fluorescent substrate. Cleavage of the substrate by caspase 3 releases the fluorocbrome, which then fluoresces green. An assay that may be employed to monitor cell proliferation associated with a TGN-associated protein is the MTT cell proliferation assay. The MTT cell proliferation assay is a colorimetric assay which measures the reduction of a tetrazolium component (MTT) into an insoluble formazan product by the mitochondria of viable cells. After incubation of the cells with the MTT reagent, a detergent solution is added to lyse the cells and solubilize the colored crystals. The samples may be read using an ELISA plate reader. The amount of color produced is directly proportional to the number of viable cells.

Additional bioassays for TGN-associated protein activities may include assays to detect the improper processing of a protein that is associated with a degenerative neurological disorder. One assay that may be used to detect TGN-associated protein activity associated with a neurological disorder is an assay to detect the presence, including an increase or a decrease in the amount, of amyloid beta protein, which is associated with Alzheimer's disease. One such assay includes assessing the effect of modulation of a TGN-associated protein on the production of amyloid beta protein. For example, the use of RNAi may be employed to knockdown the expression of a TGN-associated protein (e.g., RNAi to knockdown POSH expression) in cells (e.g., CHO cells or COS cells) that express the proteins requisite for gamma-secretase activity (including e.g., presenilin, nicastrin, Aph-1, and Pen-2), which enzymatic activity is required for the proteolytic cleavage of APP to yield amyloid beta peptide. The production of amyloid beta peptide, e.g., in the cell culture media, can then be assessed and compared to amyloid beta production from control cells, which may be cells in which the TGN-associated protein activity has not been modulated. Likewise, in vitro gamma-secretase assays may be employed on the test cells to assess the effect of modulation of a TGN-associated protein (e.g., knockdown of POSH expression) on gamma-secretase activity in comparison to the gamma-secretase activity in control cells, which are cells in which the TGN-associated protein activity has not been modulated. For example, gamma-secretase activity in the cells in which TGN-associated protein activity has been modulated (e.g., by RNAi) may be monitored by incubating solubilized gamma-secretase from the cells with tagged (e.g., a FLAG epitope) APP-based substrate and detecting the substrates and cleavage products (e.g., amyloid beta peptide) by immunoblotting and comparing the results to those of control cells (cells in which the TGN-associated protein activity has not been modulated) manipulated in the same manner. The effect of modulation of an activity of a TGN-associated protein on amyloid beta production may be assessed in any cell capable of producing amyloid beta peptide.

In certain embodiments, TGN-associated protein activities may include, without limitation, complex formation, ubiquitination and membrane fusion events (e.g., release of viral buds or fusion of vesicles). TGN-associated protein complex formation may be assessed by immunoprecipitation and analysis of co-immunoprecipiated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nr and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, the TGN-associated polypeptide and the interacting protein of interest are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (eg. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808; Levine et al. (1982) Comp. Biochem. Physiol., 72B:77-85; Tersikh et al. (2000) Science 290: 1585-88.

Other exemplary fluorescent moieties well known in the art include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri (Baldwin et al. (1990) Biochemistry 29:5509-15), peridinin-chlorophyll a binding protein from the dinoflagellate Symbiodinium sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

FRET-based assays may be used in cell-based assays and in cell-free assays. FRET-based assays are amenable to high-throughput screening methods including Fluorescence Activated Cell Sorting and fluorescent scanning of microtiter arrays.

In a further embodiment, transcript levels may be measured in cells having higher or lower levels of TGN-associated protein activity in order to identify genes that are regulated by TGN-associated proteins. Promoter regions for such genes (or larger portions of such genes) may be operatively linked to a reporter gene and used in a reporter gene-based assay to detect agents that enhance or diminish TGN-associated protein-regulated gene expression. Transcript levels may be determined in any way known in the art, such as, for example, Northern blotting, RT-PCR, microarray, etc. Increased TGN-associated protein activity may be achieved, for example, by introducing a strong TGN-associated protein expression vector. Decreased TGN-associated protein activity may be achieved, for example, by RNAi, antisense, ribozyme, gene knockout, etc.

In general, where the screening assay is a binding assay (whether protein-protein binding, agent-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

In further embodiments, the invention provides methods for identifying targets for therapeutic intervention. A polypeptide that interacts with a TGN-associated protein or participates in a TGN-associated protein-mediated process (such as viral maturation) may be used to identify candidate therapeutics. Such targets may be identified by identifying proteins that associated with TGN-associated proteins by, for example, immunoprecipitation with an anti-TGN-associated protein antibody, in silico analysis of high-throughput binding data, two-hybrid screens, and other protein-protein interaction assays described herein or otherwise known in the art in view of this disclosure. Agents that bind to such targets or disrupt protein-protein interactions thereof, or inhibit a biochemical activity thereof may be used in such an assay. Targets that may be identified by such approaches!, include: an UNC84, an MSTP28, a HERPUD1, a Cbl-b, a GOCAP1, a PTPN12, a GOSR2, a RALA, a SIAH1, an SMN1, an SMN2, a SYNE1, a TTC3, a VCY2IP1, a PKA, an EIF3S3; a GTPase (eg. Rac, Rac1, Rho, Ras); an E2 enzyme, a cullin; a clathrin; AP-1; AP-2; an HSP70; an HSP90, Brca1, Bard1, Nef, PAK1, PAK2, PAK family, Vav, Cdc42, PI3K (e.g., p85 or p110), Nedd4, src (src family), Tsg101, VASP, RNB6, WASP, N-WASP, a Gag, particularly an HIV Gag (e.g., p160); and KIAA0674, Similar to Spred-2, as well as, in certain embodiments, proteins- known to be associated with clathrin-coated vesicles, proteins involved in the protein sorting pathway and proteins involved in a Rac signaling pathway.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti- microbial agents, etc. may be used. The mixture of components are added in any order that provides for the requisite binding. Incubations are performed at any suitable temperature, typically between 4° and 40° C. Incubation periods are selected for optimum activity, but may also be optimized to facilitate rapid high-throughput screening.

In certain embodiments, a test agent may be assessed for its ability to perturb the localization of a TGN-associated protein, e.g., preventing TGN-associated protein localization to the Golgi network.

9. Methods and Compositions for Treatment of Disorders Associated with TGN-Associated Processes

In a further aspect, the invention provides methods and compositions for treatment of disorders associated with TGN-associated processes. Exemplary TGN-associated protein therapeutics of the invention include nucleic acid therapies such as for example RNAi constructs, antisense oligonucleotides, ribozyme, and DNA enzymes. Other TGN-associated protein therapeutics include polypeptides, peptidomimetics, antibodies and small molecules.

Antisense therapies of the invention include methods of introducing antisense nucleic acids to disrupt the expression of TGN-associated polypeptides or proteins that are necessary for TGN-associated protein function.

RNAi therapies include methods of introducing RNAi constructs to downregulate the expression of TGN-associated protein polypeptides or proteins that are necessary for TGN-associated protein function. In one embodiment of the application, RNAi therapies include methods of introducing RNAi constructs to downregulate the expression of POSH polypeptides or proteins that are necessary for POSH function. Exemplary RNAi therapeutics include any one of SEQ ID Nos: 15, 16, 18, 19, 21, 22, 24 and 25.

Therapeutic polypeptides may be generated by designing polypeptides to mimic certain protein domains important in the formation of TGN-associated protein complexes, such as, for example TGN-localization domains or SH3 or RING domains. For example, a polypeptide comprising a POSH SH3 domain such as for example the SH3 domain as set forth in SEQ ID No: 30 will compete for binding to a POSH SH3 domain and will therefore act to disrupt binding of a partner protein. In another embodiment, a binding partner may be Rac.

In view of the specification, methods for generating antibodies directed to epitopes of TGN-associated protein proteins are known in the art. Antibodies may be introduced into cells by a variety of methods. One exemplary method comprises generating a nucleic acid encoding a single chain antibody that is capable of disrupting a TGN-associated protein complex. Such a nucleic acid may be conjugated to an antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted TGN-associated protein complex. Nucleic acids expressing the desired single chain antibody may also be introduced into cells using a variety of more conventional techniques, such as viral transfection (e.g., using an adenoviral system) or liposome-mediated transfection.

Small molecules of the invention may be identified for their ability to modulate the formation of TGN-associated protein complexes, as described above.

10. Methods and Compositions for Treatment of Viral and Other Microbial-Related Disorders

Methods and compositions of the application for treatment of viral, bacterial, parasitic or other microbial-related disorders that are associated with a TGN-associated process contemplate modulating, mitigating, inhibiting, or preventing the onset or progression of the microbial-related disorder. The methods and compositions of the application include means to screen for and identify therapeutic targets and/or drugs for the treatment of one or more microbial-related disorders associated with a TGN-associated process. Microbial disorders associated with TGN-associated processes include any viral disorder associated with a virus discussed above (e.g., HIV, hepatitis); bacterial infections such as those associated with Cholera toxin, Escherichia coli enterotoxin, or Chlamydia trachomatis; and parasitic and other microbial infections.

In a further aspect, the invention provides methods and compositions for treatment of viral disorders, and particularly disorders caused by retroid viruses, RNA viruses and/or envelop viruses, including but not limited to retroviruses, rhabdoviruses, lentiviruses, and filoviruses. Preferred therapeutics of the invention function by disrupting the biological activity of a TGN-associated protein, such as a POSH, a POSH-pathway, or a POSH-AP polypeptide, that is involved in viral maturation. In a futher aspect, preferred therapeutics of the invention function by disrupting the biological activity of a complex comprising a TGN-associated protein in viral maturation.

In view of the teachings herein, one of skill in the art will understand that the methods and compositions of the invention are applicable to a wide range of viruses such as for example retroid viruses, RNA viruses, and envelop viruses. In a preferred embodiment, the present invention is applicable to retroid viruses. In a more preferred embodiment, the present invention is further applicable to retroviruses (retroviridae). In another more preferred embodiment, the present invention is applicable to lentivirus, including primate lentivirus group. In another preferred embodiment, the present invention is applicable to flavivirus (flaviviridae), e.g., West Nile virus. In most preferred embodiments, the present invention is applicable to Human Immunodeficiency virus (HIV), Human Immunodeficiency virus type-1 (HIV-1), Hepatitis B Virus (HBV), Human T-cell Leukemia Virus (HTLV), West Nile virus.

While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus (EIAV), the C-type retrovirus which infects pigs, the Avian Leukosis Sarcoma Virus (ALSV), the Feline Leukemia Virus (FeLV), the Feline Aids Virus, the Bovine Leukemia Virus (BLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-II), Human Immunodeficiency virus type-2 (HIV-2) and Human Immunodeficiency virus type-1 (HIV-1).

The method and compositions of the present invention are further applicable to RNA viruses, including ssRNA negative-strand viruses and ssRNA positive-strand viruses. The ssRNA positive-strand viruses include Hepatitis C Virus (HCV) and flavivirus (e.g., West Nile virus). In a preferred embodiment, the present invention is applicable to mononegavirales, including filoviruses. Filoviruses further include Ebola viruses and Marburg viruses.

Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus. Additional viruses that may be treated include herpes viruses.

In certain embodiments, the application relates to the inhibition of viral maturation by modulation of an activity associated with a Vpu polypeptide. Vpu is an HIV-1 encoded ion channel, which, among other tasks in the HIV-1 life cycle, is necessary for efficient virus budding (Schubert, U et al (1995) J. Virol. 69:7699-7711). Vpu may function at the trans Golgi network (TGN). Vpu expresses an acidic amino acid sorting motif that is required for TGN localization through a retroviral process mediated by PACS-1 (Wan, L et al (1998) Cell 94:205-216). Moreover, the phenotype conferred by human POSH knockdown is similar to that observed in cells expressing HIV-1 lacking Vpu where viruses also accumulate in intracellular membranes (Klimkait, T et al (1990) J. Virol. 64:621-629).

Vpu regulates virus release from a post-endoplasmic reticulum compartment, such as possibly the TGN, by an ion channel activity mediated by its transmembrane anchor. Vpu also induces the selective down regulation of host cell receptor proteins such as CD4 and major histocompatibility complex class I molecules, in a process involving its cytoplasmic tail. Furthermore, Vpu-mediated degradation of CD4 is dependent on an intact ubiquitin-conjugating system. (See Schubert, U et al (1998) J. Virol. 72:2280-8). In certain embodiments of the present invention, Vpu-mediated degradation of a protein such as CD4 may involve a ubiquitin-conjugating system that includes a POSH polypeptide or a POSH-AP, such as, for example, Cbl-b.

In certain aspects, the application relates to modulation of a TGN-associated process that is the processing of Vpu polypeptides. The processing of Vpu polypeptides includes the localization of Vpu to the TGN. In certain embodiments, processing of Vpu polypeptides includes the involvement of Vpu in the release of viral particles, e.g., the release of HIV viral proteins. In certain embodiments, the modulation of a TGN-associated process that is the processing of Vpu polypeptides relates to the modulation of a POSH polypeptide, such as, for example, inhibition of POSH ubiquitin ligase activity inhibits the ubiquitination of Vpu.

In certain aspects of the application, modulation of a TGN-associated process involves the modulation of the aberrant processing of a protein associated with a viral or other microbial-related disorder, and the modulation of the TGN-associated process involves modulation of a TGN-associated protein. In certain preferred embodiments, modulation of the TGN-associated process involves modulation of POSH polypeptides. In another preferred embodiment of the application, modulation of the TGN-associated process involves modulation of a POSH-AP that is Vpu.

11. Methods and Compositions for Treatment of Neurological Disorders

In a further aspect, the invention provides methods and compositions for treatment of neurological disorders that are associated with a TGN-associated process. Preferred therapeutics of the invention function by disrupting the biological activity of a complex comprising a TGN-associated polypeptide involved in the pathogenesis of a neurological disorder.

Methods and compositions of the application for treatment of neurological disorders that are associated with a TGN-associated process contemplate modulating, mitigating, inhibiting, or preventing the onset or progression of a neurological disorder. The methods and compositions of the application include means to screen for and identify therapeutic targets and/or drugs for the treatment of one or more neurological disorders associated with a TGN-associated process. Neurological disorders associated with TGN-associated processes include Alzheimer's disease, Parkinson's disease, Huntington's disease, schizophrenia, Niemann-Pick's disease, and prion-associated diseases.

In certain embodiments, the application relates to methods and compositions for the treatment of Alzheimer's disease by modulating a TGN-associated process. In certain aspects, the TGN-associated process that is modulated is the aberrant processing of proteins, which aberrantly processed proteins are associated with a neurological disorder, such as Alzheimer's disease. An aberrantly processed protein that is associated with the pathological state of Alzheimer's disease is amyloid beta precursor protein (“APP”). One of the pathological hallmarks of Alzheimer's disease is the presence of senile plaques containing amyloid beta peptide, including amyloid beta peptides ending at position 42 (Abeta42). Abeta peptides are a product of the improper proteolysis of APP. Sequential cleavage of APP occurs by proteases originally termed beta- and gamma-secretases. Beta-secretase has been identified as an aspartyl protease, BACE. The gamma-secretase mediated cleavage of APP involves the presenilins (PS), presenilin 1 (PS1) and presenilin 2 (PS2). Mutant forms of PS increase the production Abeta42.

A number of proteins which localize to the TGN are involved in the proteolysis of APP to yield Abeta peptides. For example, furin is a protein which localizes to the TGN and is required to activate BACE, which also localizes to the TGN. BACE binds the protein, nicastrin, a protein which has been shown to interact with PS and is required to impart gamma-secretase activity. Furthermore, PS proteins have been shown to interact with TGN-associated proteins, such as galactosyltransferase, Rab11, and clathrin-coated vesicles and adaptin. Additionally, PS proteins have been shown to interact with the POSH-AP, HERPUD1. Moreover, HERPUDI polypeptides have been demonstrated to be involved in the generation of Abeta peptides.

Accordingly, in certain embodiments, the present application relates to modulation of an activity of HERPUD1. In certain preferred embodiments, the application relates to modulation of an activity of HERPUD1 that involves PS1 and/or PS2. In another preferred embodiment, the application relates to modulation of an activity of HERPUD1 that involves the generation of Abeta peptides. In additional embodiments of the application, modulation of HERPUD1 involves modulation of a POSH polypeptide. In embodiments of the present application, it is contemplated that modulation of HERPUD1 will result in a decrease in the generation of Abeta peptides. In further embodiments, this decrease will be associated with a decrease in the onset and/or progression of Alzheimer's disease.

Modulation of TGN-associated processes involving aberrantly processed proteins associated with neurological disorders other than Alzheimer's disease is also contemplated by embodiments of the present application. Modulation of proteins associated with TGN-associated processes include modulation of parkin, a ubiquitin ligase associated with the pathology of Parkinson's disease; HIP14, an interacting protein of huntingtin, which is associated with Huntington disease; the protein product of the G72 gene, which is implicated in schizophrenia; and prion protein, PrP, which is associated with prion diseases.

In certain aspects of the application, modulation of a TGN-associated process involves the modulation of the aberrant processing of a protein associated with a neurological disorder, and the modulation of the TGN-associated process involves modulation of a POSH, a POSH-pathway, and/or a POSH-AP polypeptide.

12. Methods and Compositions for Treatment of Neoplastic Conditions

In a further aspect, the invention provides methods and compositions for treatment of neoplastic conditions that are associated with a TGN-associated process. Preferred therapeutics of the invention function by disrupting the biological activity of a TGN-associated protein involved in the pathogenesis of a neoplastic condition.

Methods and compositions of the application for treatment of neoplastic conditions that are associated with a TGN-associated process include modulating, mitigating, inhibiting, or preventing the onset or progression of a neoplastic condition. The methods and compositions of the application provide means to screen for and identify therapeutic targets and/or drugs for the treatment of one or more neoplastic conditions, such as cancer, associated with a TGN-associated process. Cancers associated with TGN-associated processes include thyroid carcinoma, liver cancer (hepatocellular cancer), lung cancer, cervical cancer, colorectal cancer, ovarian cancer, renal cell carcinoma, lymphoma, osteosarcoma, prostate cancer, liposarcoma, leukemia, breast carcinoma, and breast adeno-carcinoma.

Many secreted or transmembrane proteins that pass through the secretory pathway are involved in cellular proliferation. For example, the POSH-pathway polypeptide, phospholipase D (“PLD”), is a protein that passes through the secretory pathway, and PLD is associated with certain cancers. PLD mRNA and protein levels are increased in breast cancer tissues, and PLD alleles are associated with susceptibility to colorectal cancer (Noh, DY et al. (2000) Cancer Lett 161:207-14; Yamada, Y et al. (2003) J. Mol. Med. 81:126-31).

In certain embodiments of the application, the application provides methods and compositions for modulating the activity of a TGN-associated process that is the aberrant processing of a protein, which aberrantly processed protein is associated with a neoplastic condition such as cancer. In certain preferred embodiments, the aberrantly processed protein is PLD. Aberrant processing of PLD includes increased or decreased secretion of PLD polypeptides. In certain aspects of the application, modulation of the TGN-associated process of aberrant PLD processing (e.g., PLD secretion) relates to modulation of a POSH activity. In one embodiment, such modulation includes disrupting a POSH activity by inhibiting the expression of POSH polypeptides through the use of RNAi. In certain aspects of the application, modulation of the TGN-associated process of aberrant PLD processing may relate to modulation of an activity of the POSH-AP, PKA.

PLD activation involves the POSH-AP, RalA. RalA is a small GTPase that is implicated in tumor formation and cell transformation. Furthermore, increased PLD activity is associated with cellular transformation. In certain instances, the increased activation of PLD is mediated by oncogenic Ras proteins, and RalA polypeptides are members of the Ras superfamily of GTPases. Additionally, RalA polypeptides are involved in vesicle transport and interact with the exocyst complex, which is involved in exocytosis. In certain aspects of the application, the application relates to modulation of a TGN-associated process that is the aberrant processing of RalA. Aberrant processing of RalA includes disrupted or increased association of RalA with PLD. It is contemplated by embodiments of the present application that modulation of the POSH-AP, RalA, will result in decreased PLD activity, which decreased activity will be associated with the inhibition or prevention of cellular transformation that is associated with certain cancers (e.g., breast cancer, colorectal cancer).

In certain aspects of the application, modulation of a TGN-associated process involves the modulation of the aberrant processing of a protein associated with a neoplastic condition, and the modulation of the TGN-associated process involves modulation of a POSH, a POSH-pathway, and/or a POSH-AP polypeptide. In preferred embodiments of the application, the modulation of the TGN-associated process involves modulation of a POSH polypeptide, which modulation effects the activity of PLD, including the secretion of PLD. In another preferred embodiment of the application, the modulation of the TGN-associated process involves modulation of the POSH-pathway polypeptide, PLD. In yet another preferred embodiment of the application, the modulation of the TGN-associated process involves modulation of the POSH-AP, RalA.

13. Effective Dose

Toxicity and therapeutic efficacy of such compounds can be determined by standard pharmaceutical procedures in cell cultures or experimental animals, e.g., for determining The Ld₅₀ (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

14. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

An exemplary composition of the invention comprises an RNAi mixed with a delivery system, such as a liposome system, and optionally including an acceptable excipient. In a preferred embodiment, the composition is formulated for topical administration for, e.g., herpes virus infections.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

Exemplification

The invention now being generally described, it will be more readily understood by reference to the following examples, which are included merely for purposes of illustration of certain aspects and embodiments of the present invention, and are not intended to limit the invention.

### EXAMPLES

### Example 1

### Role of POSH in Virus-Like Particle (VLP) Budding

1. Objective:

Use RNAi to inhibit POSH gene expression and compare the efficiency of viral budding and GAG expression and processing in treated and untreated cells.

2. Study Plan:

5 HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA or by immunoprecipitation of target protein followed by immunoblotting.

3. Methods, Materials, Solutions

a. Methods
i. Transfections according to manufacturer's protocol and as described in procedure.
ii. Protein determined by Bradford assay.
iii. SDS-PAGE in Hoeffer miniVE electrophoresis system. Transfer in Bio-Rad mini- protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life Technologies | 11668-019 | 1112496 |
| (LF2000) | | | |
| OptiMEM | Life Technologies | 31985-047 | 3063119 |
| RNAi Lamin A/C | Self | 13 | |
| RNAi TSG101 688 | Self | 65 | |
| RNAi Posh 524 | Self | 81 | |
| plenvl1 PTAP | Self | 148 | |
| plenvl1 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life Technologies | NP0321 | 1081371 |
| Glycine SDS-PAGE gel | | | |
| Nitrocellulose | Schleicher & | 401353 | BA-83 |
| membrane | Schuell | | |
| Transfer buffer | Life Technologies | NP0006-1 | 224365 |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | Triton X-100 or NP-40 and | 1% |
| | sodium deoxycholate 0.5% | |
| | each | |
| 6X Sample | Tris-HCl, pH = 6.8 | 1 M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection I | Passage | Transfection II | Extract RNA |
| cells | (RNAi only) | cells | (RNAi and | for RT-PCR |
| | | (2:3) | pNlenv) | (post |
| | | | (12:00, PM) | transfection) |
| | | | Extract RNA for | Harvest VLPs |
| | | | RT-PCR | and cells |
| | | | (pre-transfection) | |

b. Day 1

Plate HeLa SS-6 cells in-6-well plates (35mm wells) at concentration of 5×10⁵ cells/well.

c. Day 2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | Posh 524 | 81 | 2 | 50 | 12.5 | 500 | 500 |

Transfections:

Prepare LF2000 mix: 250 μl OptiMEM+5 μl LF2000 for each reaction. Mix by inversion, 5 times. Incubate 5 minutes at room temperature.

Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.

Add 500 μl transfection mixture to cells dropwise and mix by rocking side to side.

Incubate overnight.

d. Day 3

Split 1:3 after 24 hours. (Plate 4 wells for each reaction, except reaction 2 which is plated into 3 wells.)

e. Day 4

2 hours pre-transfection replace medium with DMEM growth medium without antibiotics.

Transfection II

| | | | | | | | B | | |
|---|---|---|---|---|---|---|---|---|---|
| | | | | | | A | RNAi | | |
| | | | | | | Plasmid | [20 μM] for | C | D |
| | | | | | Plasmid | for 2.4 μg | 10 nM | OPtiMEM | LF2000 mix |
| Reaction | RNAi name | TAGDA# | Plasmid | Reactions | (μg/μl) | (μl) | (μl) | (μl) | (μl) |
| 1 | Lamin A/C | 13 | PTAP | 3 | | 3.4 | 3.75 | 750 | 750 |
| 2 | Lamin A/C | 13 | ATAP | 3 | | 2.5 | 3.75 | 750 | 750 |
| 3 | TSG101 688 | 65 | PTAP | 3 | | 3.4 | 3.75 | 750 | 750 |
| 5 | Posh 524 | 81 | PTAP | 3 | | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.

Prepare RNA+DNA diluted in OptiMEM (Transfection II, A+B+C)

Add LF2000 mix (Transfection II, D) to diluted RNA+DNA dropwise, mix by gentle vortex, and incubate 1 h while protected from light with aluminum foil.

Add LF2000 and DNA+RNA to cells, 500 μl/well, mix by gentle rocking and incubate overnight.

f. Day 5

Collect samples for VLP assay (approximately 24 hours post-transfection) by the following procedure (cells from one well from each sample is taken for RNA assay, by RT-PCR).

g. Cell Extracts
i. Pellet floating cells by centrifugation (5 min, 3000 rpm at 4° C.), save supernatant (continue with supernatant immediately to step h), scrape remaining cells in the medium which remains in the well, add to the corresponding floating cell pellet and centrifuge for 5 minutes, 1800 rpm at 4° C.
ii. Wash cell pellet twice with ice-cold PBS.
iii. Resuspend cell pellet in 100 μl lysis buffer and incubate 20 minutes on ice.
iv. Centrifuge at 14,000 rpm for 15 min. Transfer supernatant to a clean tube. This is the cell extract.
v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.

h. Purification of VLPs from cell media
i. Centrifuge supernatant at 14,000 rpm at 4° C. for at least 2 h.
ii. Filter the supernatant from step g through a 0.45 m filter.
iii. Aspirate supernatant carefully.
iv. Re-suspend VLP pellet in hot (100° C. warmed for 10 min at least) 1× sample buffer.
v. Boil samples for 10 minutes, 100° C.

i. Western Blot analysis
i. Run all samples from stages A and B on Tris-Glycine SDS-PAGE 12.5% (120V for 1.5 h.).
ii. Transfer samples to nitrocellulose membrane (65V for 1.5 h.).
iii. Stain membrane with ponceau S solution.
iv. Block with 10% low fat milk in TBS-T for 1 h.
v. Incubate with anti p24 rabbit 1:500 in TBS-T o/n.
vi. Wash 3 times with TBS-T for 7 min each wash.
vii. Incubate with secondary antibody anti rabbit cy5 1:500 for 30 min.
viii. Wash five times for 10 min in TBS-T
ix. View in Typhoon gel imaging system (Molecular Dynamics/APBiotech) for fluorescence signal.

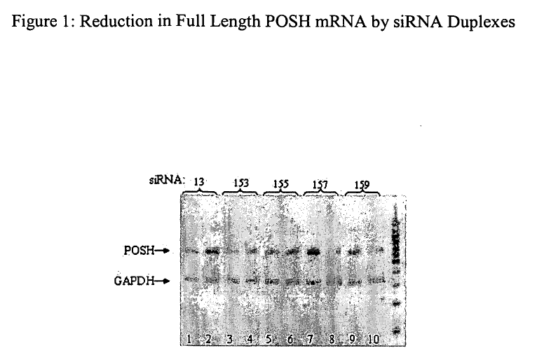

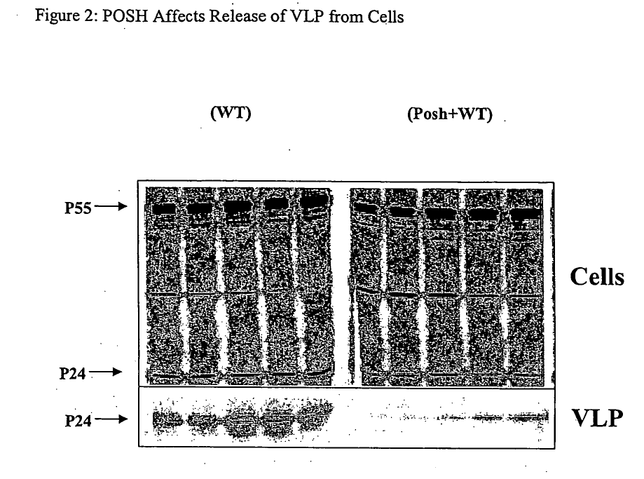

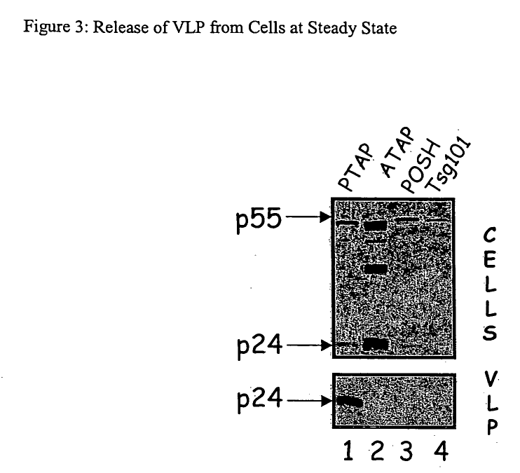

Results are shown in FIGS. 1-3.

### Example 2

### Exemplary POSH RT-PCR Primers and siRNA Duplexes

RT-PCR primers

| | | |
|---|---|---|
| Name | Position | Sequence |

| | | | |
|---|---|---|---|
| Sense primer | POSH = 271 | 271 | 5′ CTTGCCTTGCCAGCATAC 3′ (SEQ ID NO:12) |
| Anti-sense | POSH = 926c | 926C | 5′ CTGCCAGCATTCCTTCAG 3′ (SEQ ID NO:13) |
| primer | | | |

siRNA duplexes:

siRNA No: 153

siRNA Name: POSH-230

Position in mRNA 426-446

Target sequence:

SEQ ID NO: 14

5′ AACAGAGGCCTTGGAAACCTG 3′
siRNA sense strand:

SEQ ID NO: 15

5′ dTdTGAGAGGCCUUGGAAACCUG 3′
siRNA anti-sense strand:

SEQ ID NO: 16

5′ dTdTCAGGUUUCCAAGGCCUCUG 3′

siRNA No: 155

siRNA Name: POSH-442

Position in mRNA 638-658

Target sequence:

SEQ ID NO: 17

5′ AAAGAGCCTGGAGACCTTAAA 3′
siRNA sense strand:

SEQ ID NO: 18

5′ ddTdTAGAGCCUGGAGACCUUAAA 3′
siRNA anti-sense strand:

SEQ ID NO: 19

5′ ddTdTUUUAAGGUCUCCAGGGUCU 3′

siRNA No: 157

siRNA Name: POSH-U111

Position in mRNA 2973-2993

Target sequence:

SEQ ID NO: 20

5′ AAGGATTGGTATGTGACTCTG 3′
siRNA sense strand:

SEQ ID NO: 21

5′ dTdTGGAUUGGUAUGUGACUCUG 3′
siRNA anti-sense strand:

SEQ ID NO: 22

5′ dTdTCAGAGUCACAUACCAAUCC 3′

siRNA No: 159

siRNA Name: POSH-U410

Position in mRNA 3272-3292

Target sequence:

SEQ ID NO: 23

5′ AAGCTGGATTATCTCCTGTTG 3′
siRNA sense strand:

SEQ ID NO: 24

5′ ddTdTGCUGGAUUAUCUCCUGUUG 3′
siRNA anti-sense strand:

SEQ ID NO: 25

5′ ddTdTCAACAGGAGAUAAUCCAGC 3′

siRNA No.: 187

siRNA Name: POSH-control

Position in mRNA: None. Reverse to #153

Target sequence:

SEQ ID NO: 36

5′ AAGTCCAAAGGTTCCGGAGAC 3′

### Example 3

### Effects of POSH RNAi on HIV Release: Kinetics

A1. Transfections
1. One day before transfection plate cells at a concentration of 5×10⁶ cell/well in 15 cm plates.
2. Two hours before transfection, replace cell media to 20 ml complete DMEM without antibiotics.
3. DNA dilution: for each transfection dilute 62.5 μl RNAi in 2.5 ml OptiMEM according to the table below. RNAi stock is 20 μM (recommended concentration: 50 nM, dilution in total medium amount 1:400).
4. LF 2000 dilution: for each transfection dilute 50 μl lipofectamine 2000 reagent in 2.5 ml OptiMEM.
5. Incubate diluted RNAi and LF 2000 for 5 minutes at RT.
6. Mix the diluted RNAi with diluted LF2000 and incubated for 20-25 minutes at RT.
7. Add the mixure to the cells (drop wise) and incubate for 24 hours at 37° C. in CO₂ incubator.
8. One day after RNAi transfection split cells (in complete DMEM medium to 2×15 cm plate and 1 well in a 6 wells plate)
9. One day after cells split perform HIV transfection according to SP 30-012-01.
10.6 hours after HIV transfection replace medium to complete DMEM medium.
* It is important to perform RT-PCR for Posh to assure complete knockdown.

(i) A2. Total RNA purification.
1. One day after transfection, wash cells twice with sterile PBS.
2. Scrape cells in 2.3 ml/200 μl (for 15 cm plate/1 well of a 6 wells plate) Tri reaget (with sterile scrapers) and freeze in −70° C. (RNA purification and RT-PCR will be done by molecular biology unit).

RT-PCR (reverse transcription-polymerase chain reaction) is the enzymatic amplification of mRNA via the use of the enzyme, reverse transcriptase, which enables synthesis of a DNA strand complementary to the mRNA strand of interest, followed by amplification of this complementary DNA (cDNA) through employment of the polymerase chain reaction (PCR). The cDNA strand synthesized by reverse transcriptase serves as the template for the amplification of the cDNA through PCR.

RT-PCR was performed for POSH to confirm knockdown of POSH mRNA.

| | Chase | | |
|---|---|---|---|
| | time | | |
| Treatment | (hours) | Fraction | Labeling |
| a) Control = WT | 1 | Cells | A1 |
| | | VLP | A1 V |
| | 2 | Cells | A2 |
| | | VLP | A2 V |
| | 3 | Cells | A3 |
| | | VLP | A3 V |
| | 4 | Cells | A4 |
| | | VLP | A4 V |
| | 5 | Cells | A5 |
| | | VLP | A5 V |
| b) Posh + WT | 1 | Cells | B1 |
| | | VLP | B1 V |
| | 2 | Cells | B2 |
| | | VLP | B2 V |
| | 3 | Cells | B3 |
| | | VLP | B3 V |
| | 4 | Cells | B4 |
| | | VLP | B4 V |
| | 5 | Cells | B5 |
| | | VLP | B5 V |

c) B. Labeling
1. Take out starvation medium, thaw and place at 37° C.
2. Scrape cells in growth medium and transfer gently into 15 ml conical tube.
3. Centrifuge to pellet cells at 1800 rpm for 5 minutes at room temperature.
4. Aspirate supernatant and let tube stand for 10 sec. Remove the rest of the supernatant with a 200 μl pipetman.
5. Gently add 10 ml warm starvation medium and resuspend carefully with a 10 ml pipette, up and down (just turning may not resolve the cell pellet).
6. Transfer cells to 10 ml tube and place in the incubator for 60 minutes. Set an Eppendorf thermo mixer to 37° C.
7. Centrifuge to pellet cells at 1800 rpm for 5 minutes at room temperature.
8. Aspirate supernatant and let tube stand for 10 sec. Remove the rest of the supernatant with a 200 μl pipetman.
9. Cut a 200 μl tip from the end and resuspend cells (˜1.5×10⁷ cells in 150 μl RPMI without Met, but try not to go over 250 μl if you have more cells) gently in 150 μl starvation medium. Transfer cells to an Eppendorf tube and place in the thermo mixer. Wait 10 sec and transfer the rest of the cells from the 10 ml tube to the Eppendorf tube, if necessary add another 50 μl to splash the rest of the cells out (all specimens should have the same volume of labeling reaction!).
10. Pulse: Add 50 μl of ³⁵S-methionine (specific activity 14.2 μCi/μl), tightly cup tubes and place in thermo mixer. Set the mixing speed to the lowest possible (700 rpm) and incubate for 25 minutes.
11. Stop the pulse by adding 1 ml ice-cold chase/stop medium. Shake tube very gently three times and pellet cells at 600 rpm for 6 sec.
12. Remove supernatant with a 1 ml tip. Add gently 1 ml ice-cold chase/stop medium to the pelleted cells and invert gently to resuspend.
13. Chase: Transfer all tubes to the thermo mixer and incubate for the required chase time. At the end of total chase time, place tubes on ice, add 1 ml ice-cold chase/stop and pellet cells for 1 minute at 14,000 rpm. Remove supernatant and transfer supernatant to a second eppendorf tube. The cell pellet freeze at −80° C., until all tubes are ready.
14. Centrifuge supernatants for 2 hours at 14,000 rpm, 4° C. Remove the supernatant very gently, leave 20 μl in the tube (labeled as V) and freeze at −80° C. until the end of the time course.
*** All steps are done on ice with ice-cold buffers
15. When the time course is over, remove all tubes form −80° C. Lyse VLP pellet (from step 14) and cell pellet (step 13) by adding 500 μl of lysis buffer (see solutions), resuspend well by pipeting up and down three times. Incubate on ice for 15 minutes, and spin in an eppendorf centrifuge for 15 minutes at 4° C., 14,000 rpm. Remove supernatant to a fresh tube, discard pellet.
16. Perform IP with anti-p24 sheep for all samples.

Section 1.02 C. Immunoprecipitation
1. Preclearing: add to all samples 15 μl ImmunoPure PlusG (Pierce). Rotate for 1 hour at 4° C. in a cycler, spin 5 min at 4° C., and transfer to a new tube for IP.
2. Add to all samples 20 μl of p24-protein G conjugated beads and incubate 4 hours in a cycler at 4° C.
3. Post immunoprecipitations, transfer all immunoprecipitations to a fresh tube.
4. Wash beads once with high salt buffer, once with medium salt buffer and once with low salt buffer. After each spin don't remove all solution, but leave 50 μl solution on the beads. After the last spin remove supernatant carefully with a loading tip and leave ˜10 μl solution.
5. Add to each tube 20 μl 2× SDS sample buffer. Heat to 70° C. for 10 minutes.
6. Samples were separated on 12.5% SDS-PAGE.
7. Fix gel in 25% ethanol and 10% acetic acid for 15 minutes.
8. Pour off the fixation solution and soak gels in Amplify solution (NAMP 100 Amersham) for 15 minutes.
9. Dry gels on warm plate (60-80° C.) under vacuum.
10. Expose gels to screen for 2 hours and scan.

### Example 4

### Effect of siRNA Against Human POSH on Production of Infectious Virus from HeLa Cells

The following plan is according to the standard siRNA transfection protocol:

Plan:

Days

| 1 | 2 | 3 | 4 | 5.5 |
|---|---|---|---|---|
| Plate | Transfect | Split high | Transfect siRNA + | Harvest virus |
| HeLa | RNAi | (1:3) | HIV-1_{NL4-3} | And start infectivity |
| cells | | | | assay, take samples |
| | | | | for Western blot |

As siRNA the following was used:
#13 (= Lamin control)
#153 (= human POSH Ub ligase)

day 3 (one days following first siRNA transfections):
HeLa cells were harvested, split again 2:3 in fresh DMEM and seeded in T75 flasks.
day 4:
medium was removed, fresh DMEM medium was added and cells were transfected again with siRNA in combination with one of the following HIV-1 expression plasmids:
Env− (HIV-1_{NL4-3} (Adachi, A., Gendelman, H. E., Koenig, S., Folks, T., Willey, R., Rabson, A., and Martin, M. A. (1986). Production of acquired immunodeficiency syndrome-associated retrovirus in human and nonhuman cells transfected with an infectious molecular clone. J Virol 59: 284-291) with deletion in env gene, does not allow the expression of Env but other HIV-1 proteins), and VSV-G (CMV driven expression vector for vesicular stomatitis virus G protein). This combination allows single round infection as progeny viruses following infections with VSV-G pseudotyped viruses are free of Env glycoproteins.
Env+ (HIV-1_{NL4-3} wild type) for multiple round of infections.
Note: Both construct Env− and Env+ contained EGFP were cloned into the nef open reading frame of HIV-1_{NL4-3}. This way all cells transfected or infected cells with active virus gene expression can be detected by autoflourescence (based on Fukumori, T., Akari, H., Yoshida, A., Fujita, M., Koyama, A. H., Kagawa, S., and Adachi, A. (2000). Regulation of cell cycle and apoptosis by human immunodeficiency virus type 1 Vpr. Microbes Infect 2: 1011-1017; Lenardo, M. J., Angleman, S. B., Bounkeua, V., Dimas, J., Duvall, M. G., Graubard, M. B., Homung, F., Selkirk, M. C., Speirs, C. K., Trageser, C., et al. (2002). Cytopathic killing of peripheral blood CD4(+) T lymphocytes by human immunodeficiency virus type 1 appears necrotic rather than apoptotic and does not require env. J Virol 76: 5082-5093)
For control one T25 flaks was transfected with GFP-N1 (a CMV expression vector for plain GFP).
day 5:
the transfection efficiency was estimated by counting fluorescent cells in the GFP-N1 transfected culture using FACS analysis.
day 5.5:
36 hrs after second transfection, virus was harvested. Virus stocks were prepared as follows: HeLa cells were scraped and virus-containing supernatants were clarified by centrifugation (1,000×g, 5 min) and filtered through a 0.45 μm-pore-size filter to remove residual cells and debris. Stocks were aliquoted and frozen at −80° C. For biochemical analyses virions from aliquots of supernatants were pelleted (99 min, 14,000 rpm, 4° C.) and lysed (according to Ott et al., 2002). Samples of cell and virus fractions were analyzed by Western blot using anti-CA antibodies.
For infectivity assay serial dilutions of virus stocks were prepared in RPMI medium and used to infect Jurkat cells.
3 days post infection the percentage of infected cells in parallel cultures was estimated by FACS analyses. Each infection experiments were set up in 3 parallel cultures in 96 well plates (based on Bolton et al., 2002).
For control, one culture was incubated with cell culture supernatant form cell transfected with GFP-NI. No fluorescent cells were detected attesting for absence of unspecific staining of cells with GFP from the virus producer cells.

Remark: During virus production no toxic effect (other than some HIV-related cytopathic effect) was observed in human POSH transfected cultures when compared to the Lamin transfected culture.

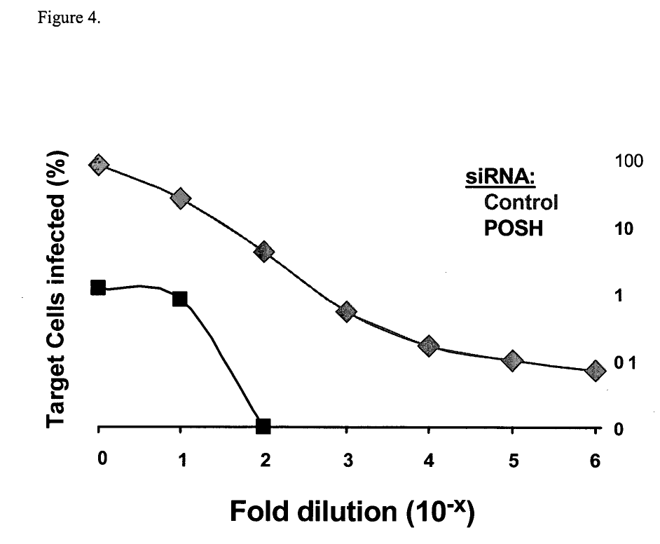

Results, shown in FIG. 4, demonstrate that knocking down POSH results in four logs reduction of HIV1 infectivity.

### Example 5

### In-Vitro Assay of Human POSH Self-Ubiquitination

Recombinant hPOSH was incubated with ATP in the presence of E1, E2 and ubiquitin as indicated in each lane. Following incubation at 37° C. for 30 minutes, reactions were terminated by addition of SDS-PAGE sample buffer. The samples were subsequently resolved on a 10% polyacrylamide gel. The separated samples were then transferred to nitrocellulose and subjected to immunoblot analysis with an anti ubiquitin polyclonal antibody. The position of migration of molecular weight markers is indicated on the right.

Poly-Ub: Ub-hPOSHconjugates, detected as high molecular weight adducts only in reactions containing E1, E2 and ubiquitin. hPOSH-176 and hPOSH-178 are a short and a longer derivatives (respectively) of bacterially expressed hPOSH; C, control E3

Experimental Procedure:

Materials

1. E1 recombinant from bacculovirus

2. E2 UbcH5c from bacteria

3. Ubiquitin

4. POSH #178 (1-361) GST fusion

5. POSH # 176 (1-269) GST fusion

6. hsHRD1 ring-containing region

5. Buffer 12 (40 mM Tris-HCl, pH 7.6, DTT 1 mM, MgCl₂ 5 mM, ATP 2 uM)

6. Dilution buffer (40 mM Tris-HCl, pH 7.6, DTT 1 mM, ovalbumin 1 ug/ul) protocol

| | 0.1 ug/ul | 0.5 ug/ul | 5 ug/ul | 0.4 ug/ul | 2.5 ug/u/ | 0.8 ug/ul | |
|---|---|---|---|---|---|---|---|
| | E1 | E2 | Ub | 176 | 178 | Hrd1 | Bx12 |
| −E1 (E2 + 176) | — | 0.5 | 0.5 | 1 | — | — | 10 |
| −E2 (E1 + 176) | 1 | — | 0.5 | 1 | — | — | 9.5 |
| −ub (E1 + E2 + 176) | 1 | 0.5 | — | 1 | — | — | 9.5 |
| E1 + E2 + 176 + Ub | 1 | 0.5 | 0.5 | 1 | | — | 9 |
| −E1 (E2 + 178) | — | 0.5 | 0.5 | — | 1 | — | 10 |
| −E2 (E1 + 178) | 1 | — | 0.5 | — | 1 | — | 9.5 |
| −ub (E1 + E2 + 178) | 1 | 0.5 | — | — | 1 | — | 9.5 |
| E1 + E2 + 178 + Ub | 1 | 0.5 | 0.5 | — | 1 | —1 | 9 |
| Hrd1, E1 + E2 + Ub | 1 | 0.5 | 0.5 | — | — | 1 | 8.5* |

1. Incubate for 30 minutes at 37° C.
2. Run 12% SDS PAGE gel and transfer to nitrocellulose membrane
3. Incubate with anti-Ubiquitin antibody.

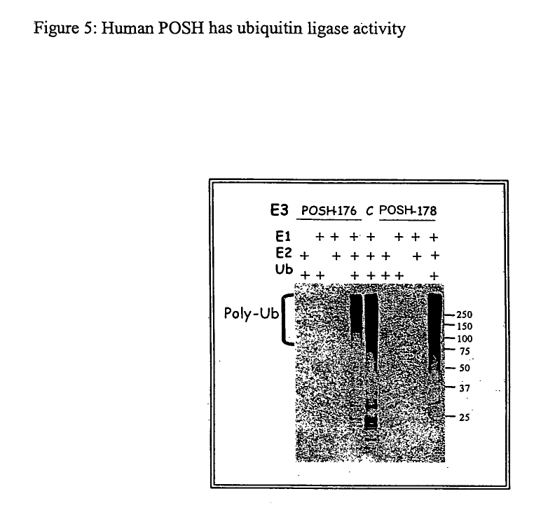

Results, shown in FIG. 5, demonstrate that human POSH has ubiquitin ligase activity.

### Example 6

### Co-Immunoprecipitation of hPOSH with myc-tazged Activated (V12) and Dominant-Negative (N17) Rac1

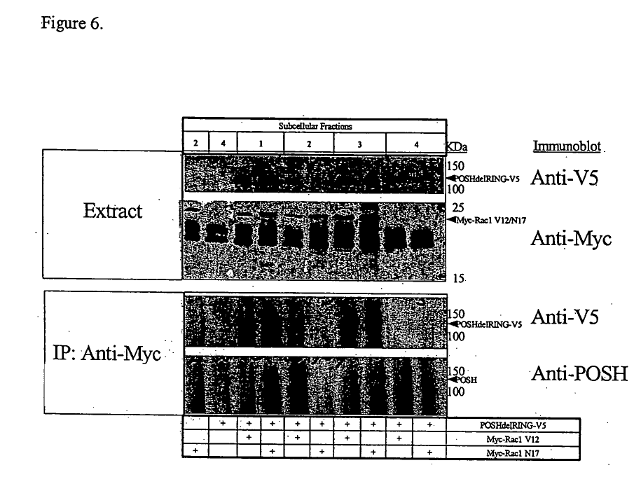

Hela cells were transfected with combinations of myc-Rac1 V12 or N17 and hPOSHdeIRING-V5. 24 hours after transfection (efficiency 80% as measured by GFP) cells were collected, washed with PBS, and swollen in hypotonic lysis buffer (10 mM HEPES pH=7.9, 15 mM KCl, 0.1 mM EDTA, 2 mM MgCl2, 1 mM DTT, and protease inhibitors). Cells were lysed by 10 strokes with dounce homogenizer and centrifuged 3000×g for 10 minutes to give supernatant (Fraction 1) and nucleii. Nucleii were washed with Fraction 2 buffer (0.2% NP-40, lOmM HEPES pH=7.9, 40 mM KCl, 5% glycerol) to remove peripheral proteins. Nucleii were spun-down and supernatant collected (Fraction 2). Nuclear proteins were eluted in Fraction 3 buffer (20 mM HEPES pH=7.9, 0.42M KCl, 25% glycerol, 0.1 mM EDTA, 2 mM MgCl2, 1 mM DTT) by rotating 30 minutes in cold. Insoluble proteins were spun-down 14000×g and solubilized in Fraction 4 buffer (1% Fos-Choline 14, 50 mM HEPES pH=7.9, 150 mM NaCl, 10% glycerol, 1 mM EDTA, 1.5 mM MgCl2, 2 mM DTT). Half of the total extract was pre-cleared against Protein A sepharose for 1.5 hours and used for IP with 1 μg anti-myc (9E10, Roche 1-667-149) and Protein A sepharose for 2 hours. Immune complexes were washed extensively, and eluted in SDS-PAGE sample buffer. Gels were run, and proteins electro-transferred to nitrocellulose for immunoblot as in FIG. 6. Endogenous POSH and transfected hPOSHdelRING-V5 are precipitated as a complex with Myc-Rac1 V12/N17. Results, shown in FIG. 6, demonstrate that POSH co-immunoprecipitates with Rac1.

### Example 7

### Knock-Down of hPOSH Entraps HIV Virus Particles in Intracellular Vesicles

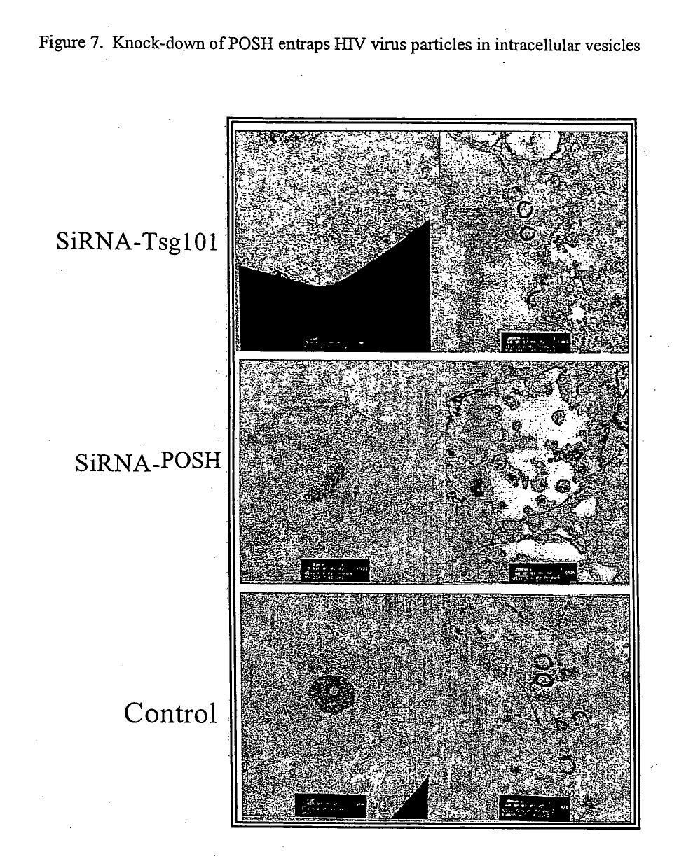

HIV virus release was analyzed by electron microscopy following siRNA and full-length HIV plasmid (missing the envelope coding region) transfection. Mature viruses were secreted by cells transfected with HIV plasmid and non-relevant siRNA (control, lower panel). Knockdown of Tsg101 protein resulted in a budding defect, the viruses that were released had an immature phenotype (upper panel). Knockdown of hPOSH levels resulted in accumulation of viruses inside the cell in intracellular vesicles (middle panel). Results, shown in FIG. 7, indicate that inhibiting hPOSH entraps HIV virus particles in intracellular vesicles. As accumulation of HIV virus particles in the cells accelerate cell death, inhibition of hPOSH therefore destroys HIV reservoir by killing cells infected with HIV.

### Example 8

### POSH is Localized to the Trans-Golgi (TGN)

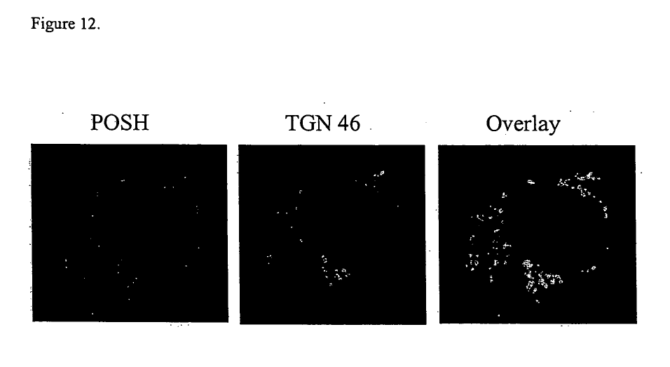

HeLa SS6 cells were incubated for 1 h at 40° C. to block vesicular transport. Cells were than fixed in 3.7% paraformaldehyde and permeablized. Cells were co-stained with sheep anti-TGN46 antibody (green) and with rabbit anti-POSH antibody (red) (see FIG. 12).

### Example 9

### POSH Localization and Golgi Function

Immunofluorescense Analysis of POSH Localization in Hela SS6 Cells:

Hela SS6 cells were plated in 6 wells containing glass bottom. 24 hours later the cells were rinsed with PBS at room temperature and fixed with 3.7% glutaraldehyde in PBS for 15 min at room temperature. Cells were permeabilized in 0.5% Triton X-100 in PBS for 15 minutes and washed in PBS +0.01% Tween-20, 3-times for 5 min each.

Cells were incubated in blocking solution [3.7% paraformaldehyde, 4% BSA, 0.01% tween20, 1% NGS (goat serum), PBS pH 7.5] for 30 min to block nonspecific binding of the antibodies.

Cells were incubated with primary antibody. in 1:10 blocking solution in PBS for 2 hours at room temperature [Rabbit anti-POSH, antibody 15A, 1:400 dilution; mouse anti-phospholipase D (PLD; BD Transduction Laboratories), 1:250 dilution; mouse anti-GM130, 1:1000 dilution (BD Transduction Laboratories)].

Wash with 1:10 diluted blocking solution in PBS.

Incubate with secondary antibody in 1:10 diluted blocking solution in PBS for 2 hours at room temperature in the dark [anti-mouse FITC, 1:250 (Jackson laboratories); anti-rabbit Rhodamine (Jackson laboratories)].

Wash with 1:10 diluted blocking solution in PBS, 3-times for 10 min each.

Staining was visualized by confocal microscopy (Zeiss 510).

POSH Localization Post-Treatment with the Golgi-Disrupting Agent BFA:

Hela SS6 cells were plated in 6 wells containing glass bottom. 24 hours later the cells were either: Rinsed with PBS at room temperature—control (a), treated for 45 minutes with 1 uM of BFA (b), treated for 24 hours with siRNA against POSH—siRNA(c) and transfected with PLenv1 plasmid-(HIV) (d).

Cells were then fixed and blocked with 3.7% paraformaldehyde, 4% BSA, 1% NGS (goat serum), PBS pH 7.5] for 30 min. Cells were permeabilized in 0.5% Triton X-100 in PBS for 15 minutes and washed in PBS. Cells were incubated with primary antibody in 1:10 blocking solution in PBS+0.01%Tween 20 for 2 hours at RT [Rabbit anti-POSH, antibody 15A, 1:400 dilution; mouse anti-phospholipase D (PLD; BD trunsduction laboratories), 1:250 dilution; mouse anti GM130 1:1000 dilution (BD trunsduction laboratories)]. Cell were then incubated with secondary antibody in 1:10 diluted blocking solution in PBS for 2 hours at room temperature in the dark [anti-mouse FITC, 1:250 (Jackson laboratories); anti-rabbit Rhodamine (Jackson laboratories)].

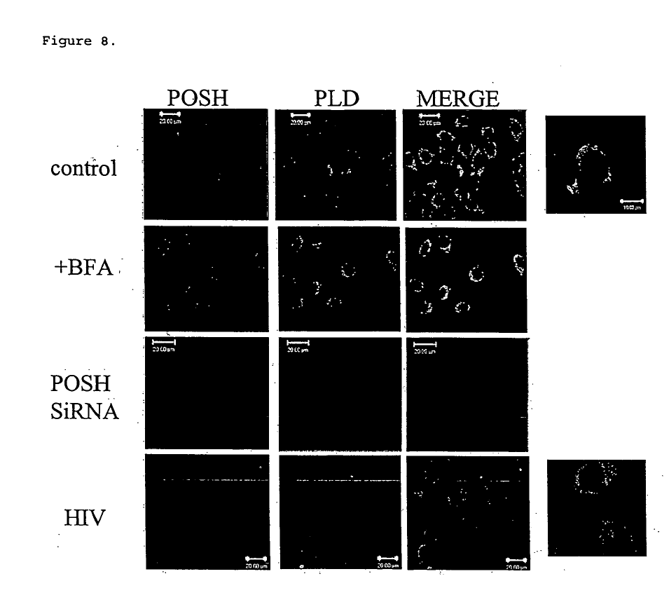

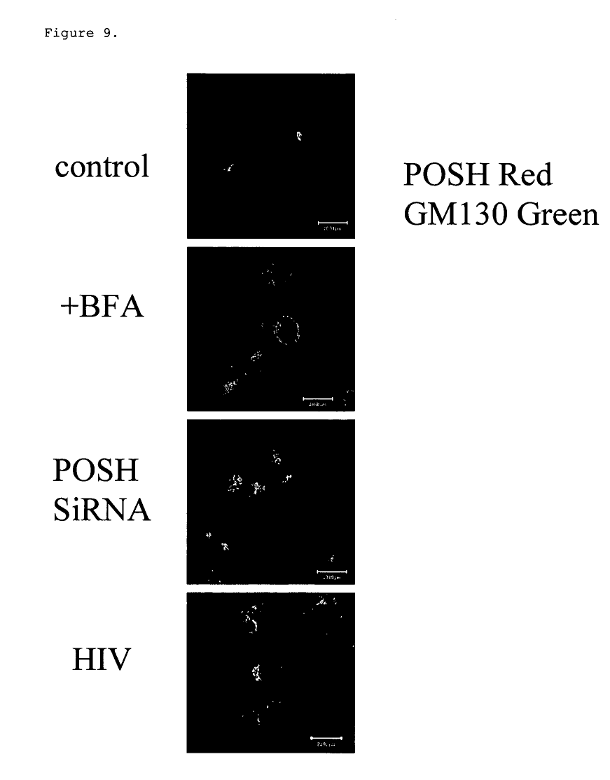

Results (FIGS. 8 and 9):

a. POSH is localized at the trans-Golgi network not with a cis-Golgi marker (co-localization with PLD and not GM130).

b. BFA treatment, which causes the dispersal of Golgi stacks into small vesicles but has little effect on the TGN, did not alter the co-localization of POSH localization with PLD.

c. The depletion of POSH (siRNA) changes the distribution pattern of PLD indicating an involvement of POSH in TGN sorting mechanism.

d. HIV transfection changes POSH localization.

e. POSH localization is punctuate in form.

### Example 10

### POSH is Localized at the TGN, a Biochemical Analysis

By using biochemical fractionation of HEK 293T cells we found that POSH is associated peripherally with the TGN membrane. A more significant amount of POSH was associated with the light density membrane in cells incubated on ice for 45 minutes prior to cell lysis. Incubation of cells at 40C blocks release of vesicles from the TGN, thus indicating that the association of POSH with the TGN is regulated by trafficking, a common theme for other peripherial TGN proteins.

SN1790—Experimental Outline
1. Three flasks of HEK 293T (ATCC) were grown to confluence and scraped into growth medium (DMEM containing 10% fetal calf serum). One sample was incubated at 4OC; a second sample was incubated at 37° C.
2. Cells were washed extensively with ice-cold PBS and pelleted at 500×g for 10 minutes; all subsequent steps were done at 4° C. with ice-cold buffers. Cell pellets were resuspended in HB buffer (0.25M sucrose, 10 mM HEPES-KOH, pH=7.5, 1 mM EDTA containing protease inhibitors). Cells were lysed by fifteen strokes of dounce homogenizer and spun at 16,000×g to pellet cellular debris and nuclei. Supernatants were transferred to ultracentrifuge tubes and re-centrifuged at 48,000×g for 20 minutes to pellet light heavy microsomes. Supernatants were transferred to fresh tubes and centrifuged at 212,000×g for 70 minutes to pellet light microsomes (Trans-golgi network and endosomes). The pellet was resuspended in HB containing 0.5% NP-40 and 0.5% sodium deoxycholate. The 212,000×g pellet and supernatant was subjected to immunopercipitation with rabbit anti-POSH antibodies directed to amino-acids 285-430. Immunoprecipitated material was separated on 7.5% SDS-PAGE and transferred to nitrocellulose and immunoblotted with rabbit antibodies directed to POSH residues 731-888.

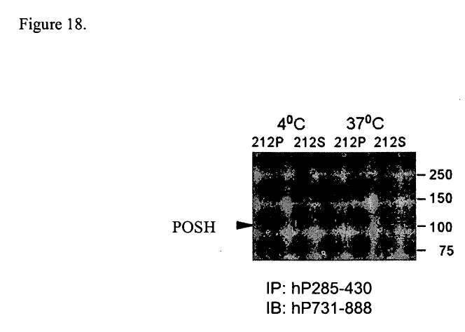

Results are presented in FIG. 18.

### Example 11

### POSH Reduction Results in Decreased Secretion of Phospholipase D (PLD)

Hela SS6 cells (two wells of 6-well plate) were transfected with POSH siRNA or control siRNA (100 nM). 24 hours later each well was split into 5 wells of a 24-well plate. The next day cells were transfected again with 100 nM of either POSH siRNA or control siRNA. The next day cells were washed three times with 1×PBS and than 0.5 ml of PLD incubation buffer (118 mM NaCl, 6 mM KCl, 1 mM CaCl₂, 1.2 mM MgSO4, 12.4 mM HEPES, pH7.5 and 1% fatty acid free bovine serum albumin) were added.

48 hours later medium was collected and centrifuged at 800×g for 15 minutes. The medium was diluted with 5×PLD reaction buffer (Amplex red PLD kit) and assayed for PLD by using the Amplex Red PLD kit (Molecular probes, A-12219). The assay results were quantified and presented below in as a bar graph.

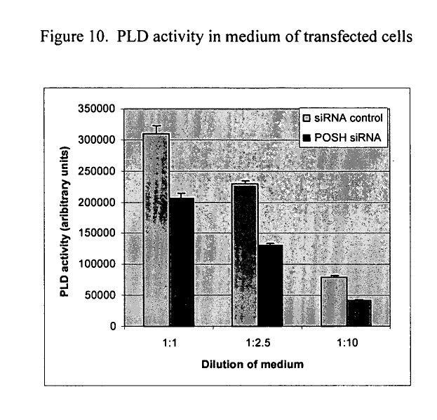

The cells were collected and lysed in 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) for 15 minutes on ice. Lysates were cleared by centrifugation and protein concentration was determined. There were equal protein concentrations between the two transfectants. Equal amount of extracts were immunoprecipitated with anti-POSH antibodies, separated by SDS-PAGE and immunoblotted with anti-POSH antibodies to assess the reduction of POSH levels. There was approximately 40% reduction in POSH levels (FIG. 10).

### Example 12

### POSH Protein Expression Profile

A. Expression Profile of POSH in Different Cell Lines.

Cells from various cell lines were lysed and cell lysates were separated by SDS-PAGE. POSH expression was analyzed by Western blot analysis using anti-POSH.

B. Tissue-Specific Expression of POSH Protein.

SDS-PAGE fractionated tissue blots (Genotech, cat no.TB54, TB37) of normal (lower blot) and tumorous (upper blot) tissues (75 μg protein each lane) were probed with rabbit polyclonal antibody 6564 (raised against POSH specific peptide) and detected by Enhanced Chemiluminescence using anti-rabbit horseradish peroxidase (Amersham cat no. NA934V) and exposure to film (Kodak Biomax). Films were digitized by table-top scanning (UMAX) and Adobe Photoshop 5.5 graphics program.

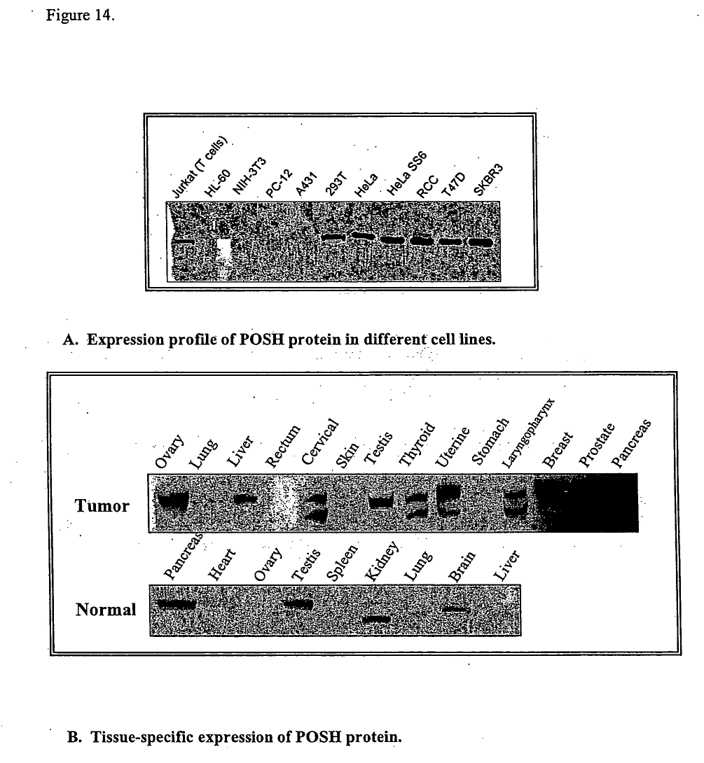

Results are shown in FIG. 14.

### Example 13

### POSH Modulates HeLa Cell Growth

HeLa cells subject to siRNA knockdown of POSH expression showed a reversible decrease in cell growth.

Protocol:

Day 1: Plate two 6-well plates with HeLa SS6 cells (5×10⁵) cells/well.

Day 2: Change medium to medium without antibiotics.

Day 3: Perform the same transfection as noted below.

Transfection: 10 nM RNAi: A2=RNAi #153; A3=RNAi#187 (see Example 2 for RNAi information)

Prepare mix of LF2000+OptiMEM: 3.25 ml optimem with 65 ul LF2000, incubate together for 5 min. Add to reactions as noted above. Incubate for 25 min. Add 500 ul to the corresponding well.

| | | | | | Mix |
|---|---|---|---|---|---|
| Reaction | No. of | RNAi | | OptiMEM | (LF2000 + |
| name | reactions | oligo # | RNAi (ul) | (ul) | OptiMEM) |
| A2 | 4 | 153 | 10 nM = 5 ul | 1000 | 1000 |
| A3 | 4 | 187 | 10 nM = 5 ul | 1000 | 1000 |

Day 4: Harvest one of each reaction for both protein extraction and RNA. (#153 day 4 and #187 day 4). For the other set of reactions: count and split each well into (5×10⁵ cells) for transfection next day.

Day 5: Perform the same transfection as noted above.

Day 6: Harvest one plate for protein and RNA extraction and the other split to (5×10⁵ cells) and (2.5×10⁵ cells)

Day 8: Harvest and count and to the (5×10⁵ cells) plate form day six.

Day 10: Harvest and count the (2.5×10⁵ cells) plate from day 6.

Protein extract: for POSH expression evaluation

1. Wash cells twice with cold PBS on ice.

2. Add PBS and scrape cells off to eppendorf tubes.

3. Centrifuge at 40 deg. C at 1800 rpm for 5 min.

4. Add 100 ul of RIPA Buffer+protease inhibitors (PI3K) (1:200)+EDTA (1:100) to the remaining cells on plate.

5. Incubate on ice for 15 minutes.

6. Centrifuge for 10 min in 40 deg. C at maximum speed.

7. Proteins (15 ug/lane) were loaded onto a 10% tris glycine gel, resolved by electrophoresis, transferred to nitrocellulose membrane and probed with anti POSH antibody for 1 h (1:2000 dilution). Detected was by ECL.

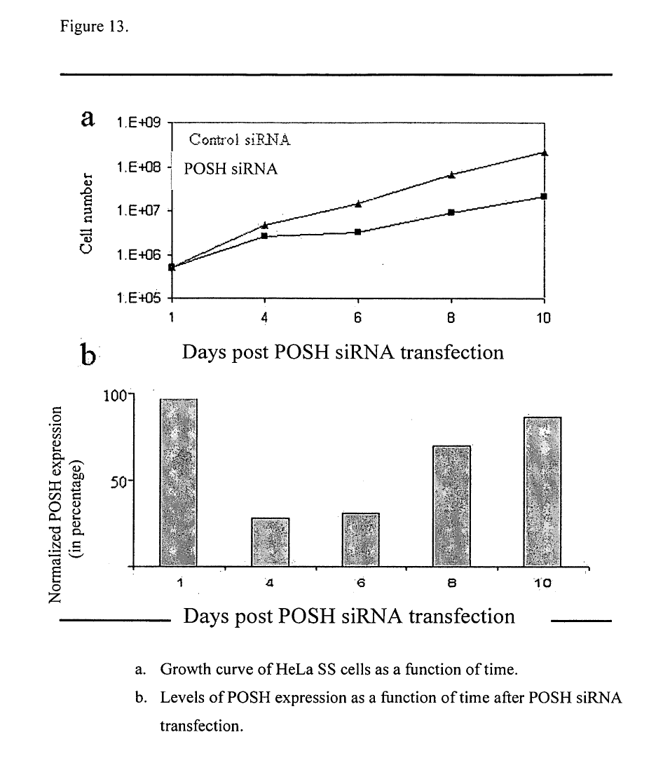

8. Evaluations of protein levels were performed using an ImageQuant analyzer. Results are shown in FIG. 13.

### Example 14

### POSH Expression in Thyroid Carcinoma

Various normal and tumor tissue sections were analyzed by immunohistochemistry for the presence of POSH. For screening of various normal and cancer samples, a human low-density normal tissue array (InnoGenex catalog # TS4201-05) and a human low-density cancer tissue array (InnoGenex catalog # TS4204-05) were used.

1. Immunohistochemistry protocol for POSH

Reagent
TBS×10 (10×25 mM TBS pH 7.5)
160 gr NaCl
60 gr Trizma base
4 gr KCl
Fill up to 2 liters final volume with ddH₂O
Adjust pH to 7.5 with concentrated HCl (ab. 32 ml)
Autoclave

TBST
Add 1 ml Tween-20 to 2 liters of TBS×1 buffer to a final concentration of 0.05%

EDTA 1 mM pH 8
Stock solution ×100
37.2 gr Disodium salt EDTA in 1000 ml H₂O Autoclave
Dilute 1:100 and adjust the pH to 8 just before using it.

Other Reagents
1. 3% hydrogen peroxide
2. Endogenous Avidin/Biotin Blocking (Zymed 00-4303 kit)
3. CasBlock (Zymed 00-8120)
4. normal swine serum (Dako)
5. DAKO Envision Plus system, HRP (AEC) kit K4009 (Rabbit).
6. Hematoxyline (Pioneer Research Chemicals)
7. Hydromount (National Diagnostics HS-106)

Staining Protocol

1. Deparaffinization and rehydration with xylene and graded alcohol (hood is required).

Prepare 3 bathes with xylene, 2 with absolute ethanol, 2 with 96% ethanol and 2 with 70% ethanol and incubate as follows:

Xylene—3min×3)
Ethanol absolute—2 min×2
Ethanol 96%—2 min×2
Ethanol 70%—2 min×2
Tap water×3 (can be done in the sink, take care not to wash with running water directly on slides)

2. Microwave Pretreatment

Transfer slides to a microwavable plastic holder. Put the slides in a 1000 ml glass beaker containing 600 ml of 1 mM EDTA, pH 8.0.

Note: When staining few slides, the holder should be filled with empty glass slides up to 20 slides in order to keep the same heating conditions in all experiments.

Heating protocol: use 60% power for 30 min (it takes about 10 min to bring to a boil and then 20 min with boiling).

Cooling: Keep microwave door half open for 5 min. Do not take beaker out of oven. Take beaker out of oven, fill it up to 1000 ml with ddH₂O and wait 10 min.

Note: It is possible to stop staining here for up to overnight. Leave slides in the beaker.

3. Wash with TBS/T 3 min×3 (first wash can be shorter) in 3 glass bathes.

4. Peroxidase blocking
Prepare fresh 200 ml of 3% hydrogen peroxide in methanol.
Incubate at room temperature for 10 minutes.

5. Wash with TBS/T 3 min×3
Endopenous Avidin/Biotin Blocking (Zymed 00-4303 kit)
a. Place paper towel in slide box (for 100 slides) and absorb it with water.
b. Level the box.
c. Wipe excess TBST from each slide
d. Place slide on box (face up)—work with 3-5 slides at a time. Avoid letting them get dried.
e. Apply 2 drops (100 μl) of reagent A (Avidin solution) to the tissue
f. Incubate 10 min at RT.
g. Wash with TBS/T 3 min×3
h. Repeat steps c. to g. with reagent B (d-Biotin solution)

6. Block with blocking solution for 60 minutes. Use CasBlock (Zymed 00-8120) containing 5% normal swine serum (Dako).

7. Wipe the slide from excess blocking solution (do not rinse with TSBT as before)

8. Add Primary antibody diluted 1:400 in blocking solution, cover with cover slide and incubate overnight at 4° C.

9. Discard cover slide and wash with TBS/T 3 min×3.

10. Secondary antibody

Use DAKO Envision Plus system, Peroxidase (AEC) kit:
a. Apply 2 drops of solution 2 (Labelled polymer) to the tissue; Apply 2 drops of solution B (biotinylated secondary antibody) to the tissue
b. Incubate 30 min at RT.
c. Wash with TBS/T 3 min×3
d. Wipe excess TBST from each slide

11. Staining with DAKO AEC Substrate kit
DAKO Envision Plus system Peroxidase (AEC) kit
Apply 2 drops of solution 3 (AEC+substrate-Chromogen) to the tissue Incubate 5-30 min. Staining development should be controlled under microscope.

12. Wash several times in tap water

13. Counterstain with Hematoxyline (4 min)

14. Wash several times in tap water

15. Cover with 2-3 drops of Hydromount. Do not wipe the slide before adding the Hydromount

16. Cover with cover-slide and let dry at room temperature.

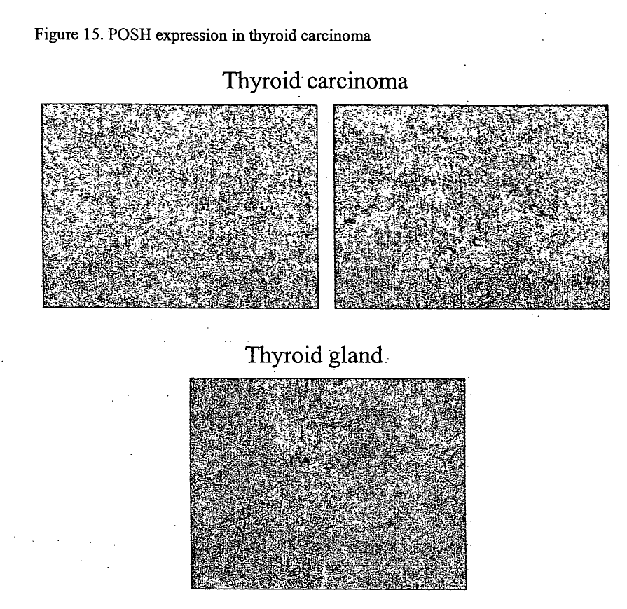

Results, as shown in FIG. 15, demonstrate that POSH is differentially expressed in thyroid carcinoma versus normal tissue.

### Example 15

### POSH Expression in Tumors

Various tumor tissue sections were analyzed by immunohistochemistry for the presence of POSH. For screening of various tumor samples a human low-density cancer tissue array (InnoGenex catalog # TS4204-05) were used.

Paraffin embedded tissues were probed with anti-POSH antibodies. A secondary alakaline phosphatase conjugated anti-rabbit antibody was used to detect POSH-bound primary antibody. The presence of red color marks the detection of POSH in the tissue section.

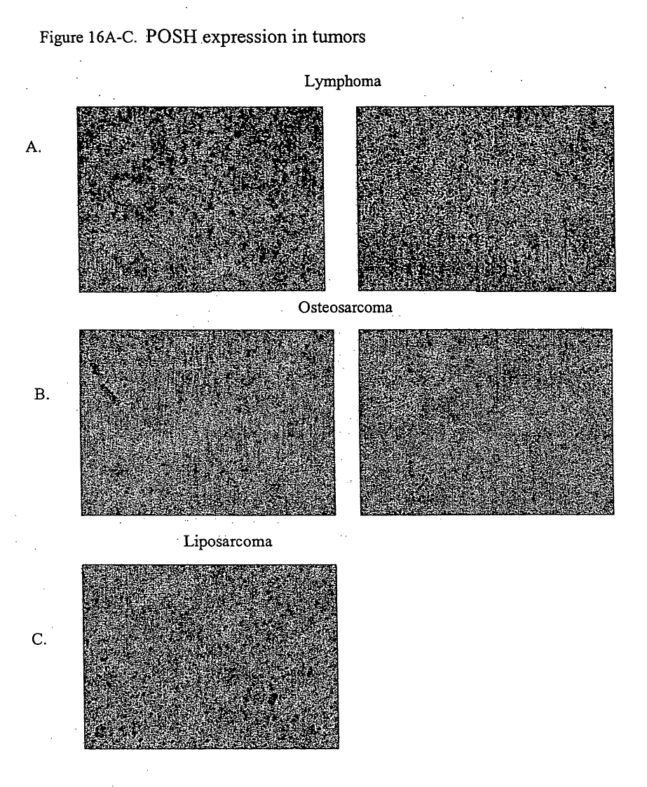

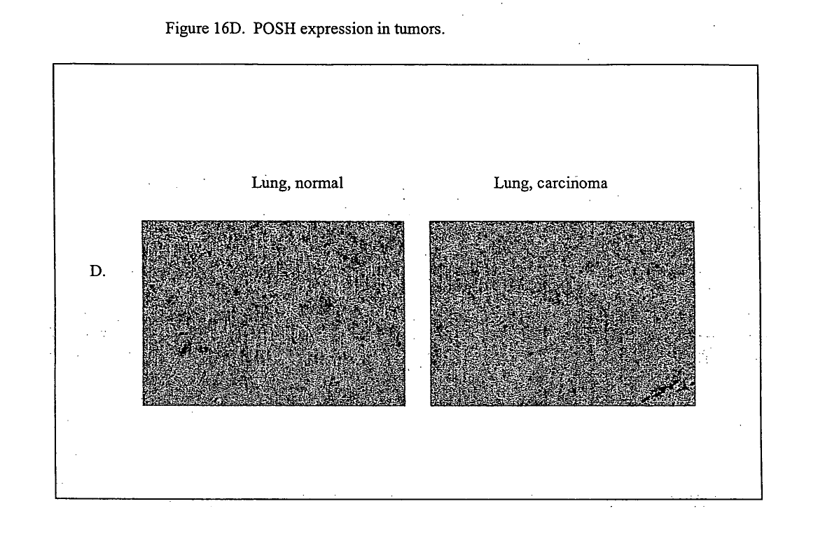

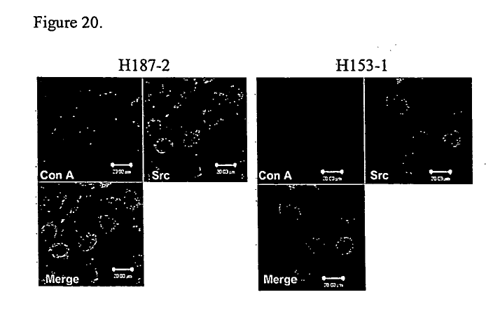

FIG. 16 shows the immunohistochemistry of human tumor tissue sections: A. Lymphoma B. osteosacoma C. liposarcoma D.normal lung (left panel), lung carcinoma (right panel). As FIG. 20 shows, POSH expression is detected in lymphoma, osteosarcoma, lippsarcoma and lung carcinoma.

### Example 16

### Analysis of the Effects of POSH Knockdown on M-MuLV Expression and Budding

Protocol:

Transfections:—

A day before transfection, Hela SS6 cells were plated in two 6 wells plates at 5×10⁵ cells per well. 24 hours later the following transfections were performed:

4 wells were transfected with control siRNA and a plasmid encoding MMuLV.

4 wells were transfected with POSH siRNA and a plasmid encoding MMuLV.

1 well was a control without any siRNA or DNA transfected.

1 well was transfected with a plasmid encoding MMuLV.

For each well to be transfected 100 nM (12.5 μl) POSH siRNA or 100 nM (12.5 μl) control siRNA were diluted in 250 μl Opti-MEM (Invitrogen). Lipofectamin 2000 (5 μl) (Invitrogen, Cat. 11668-019) was mixed with 250 μl of OptiMEM per transfected well. The diluted siRNA was mixed with the lipofectamin 2000 mix and the solution incubated at room temperature for 30 min. The mixture was added directly to each well containing 2 ml DMEM +10% FBS (w/o antibiotics).

24 hours later, four wells of the same siRNA treatment were split to eight wells, and two wells without siRNA were split to four wells.

24 hours later all wells were transfected with 100 nM control siRNA or 100 nM POSH siRNA with or without a plasmid encoding MMuLV (see table). 48 hours later virions and cells were harvested.

| | | Amount | | | |
|---|---|---|---|---|---|
| | | of RNAi | | The volume of | |
| No | | (μl) per | Amount of DNA | DNA (μl) per | |
| of wells | RNAi | well | (μg) per well | well | Application |
| 5 | POSH | 12.5 | MMuLV (2 μg) | 10 | 4 wells for |
| | 100 nM (1^{st} | | | | VLPs assay |
| | and 2^{nd} | | | | and 1 well for |
| | transfection) | | | | RT |
| 5 | Control | 12.5 | MMuLV (2 μg) | 10 | 4 wells for |
| | 100 nM (1^{st} | | | | VLPs assay |
| | and 2^{nd} | | | | and 1 well for |
| | transfection) | | | | RT |
| 1 | — | — | — | 10 μl H₂O | VLPs assay |
| 1 | — | — | MMuLV (2 μg) | 10 | VLPs assay |

Steady State VLP Assay

Cell Extracts:—
1. Pellet floating cells by centrifugation (10 min, 500×g at 4° C.), save supernatant (continued at step 7), wash cells once, scrape cells in ice-cold 1×PBS, add to the corresponding cell pellet and centrifuge for 5 min 1800 rpm at 4° C.
2. Wash cell pellet once with ice-cold 1×PBS.
3. Resuspend cell pellet in 150 μl 1% Triton X-100 lysis buffer (20 mM HEPES-NaOH, pH 7.4, 150 mM NaCl, 1.5 mM MgCl₂, 1 mM EDTA, 1% Triton X-100 and 1× protease inhibitors) and incubate 20 minutes on ice.
4. Centrifuge at 14,000 rpm for 15 min. Transfer supernatant to a clean tube.
5. Determine protein concentration by BCA.
6. Prepare samples for SDS-PAGE by adding 2 μl of 6×SB to 20 μg extract (add lysis buffer to a final volume of 12 μl), heat to 80° C. for 10 min.

Purification of virions from cell media
7. Filtrate the supernatant through a 0.45 μm filter.
8. Transfer 1500 μl of virions fraction to an ultracentrifuge tube (swinging rotor).
9. Add 300 μl of fresh sucrose cushion (20% sucrose in TNE) to the bottom of the tube.
10. Centrifuge supernatant at 35000 rpm at 4° C. for 2 h.
11. Resuspend virion pellet in 50 μl hot 1× sample buffer each (samples 153-1, 2, 3, 187-1, 2, 3). Resuspend VLPs pellet (1534, 5 and 187 4, 5) in 25 μl hot 1× sample buffer. Vortex shortly, transfer to an eppendorf tube, unite VLPs from wells 153-4+5 and 187-4+5. Heat to 80° C. for 10 min.
12. Load equal amounts of VLPs relatively to cells extracts amounts.

Western Blot Analysis
1. Separate all samples on 12% SDS-PAGE.
2. Transfer samples to nitrocellulose membrane (100V for 1.15 h).
3. Dye membrane with ponceau solution.
4. Block with 10% low fat milk in TBS-T for 1 hour.
5. Incubate membranes with Goat anti p30 (81 S-263) (1:5000) in 10% low fat milk in TBS-T over night at 4° C. Incubate with secondary antibody rabbit anti goat-HRP 1:8000 for 60 min at room temperature.
6. Detect signal by ECL reaction.
7. Following the ECL detection incubate memebranes with Donkey anti rabbit Cy3 (Jackson Laboratories, Cat 711-165-152) 1:500 and detect signal by Typhoon scanning and quantitate.

Results:

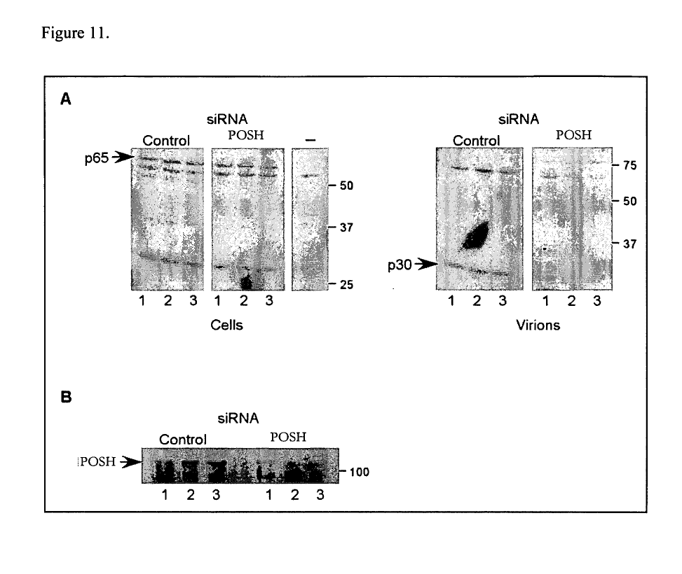

As shown in FIG. 11, POSH knockdown decreases the release of extracellular MMuLV particles.

### Example 17

### Effect of hPOSH on Gag-EGFP Intracellular Distribution

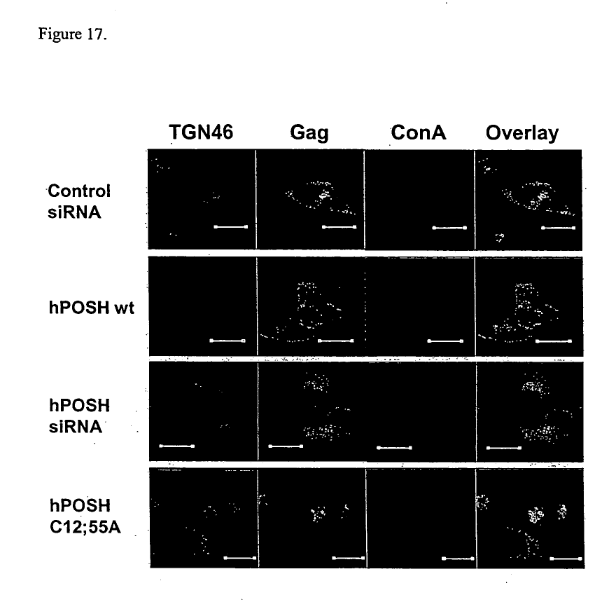

HeLa SS6 were transfected with Gag-EGFP, 24 hours after an initial transfection with either hPOSH-specific or scrambled siRNA (control) (100 nM) or with plasmids encoding either wild type hPOSH or hPOSH C(12,55)A. Fixation and staining was preformed 5 hours after Gag-EGFP transfection. Cells were fixed, stained with Alexa fluor 647-conjugated Concanavalin A (ConA) (Molecular Probes), permeabilized and then stained with sheep anti-human TGN46. After the primary antibody incubation cells were incubated with Rhodamin-conjugated goat anti-sheep. Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown. See FIG. 17.

The E3 activity of hPOSH is required for the plasma membrane localization of Gag

Inasmuch as budding at the cell surface was blocked in the absence of hPOSH, and as hPOSH associated exclusively with the TGN, we hypothesized that hPOSH might function in a transport/sorting process of Gag and Gag-pol necessary for virus assembly at the plasma membrane. To test this hypothesis we analyzed by immunofluorescence the effect of hPOSH on the cellular distribution of Gag-green fluorescent protein (GFP). In the control cells, Gag was associated with the plasma membrane and with the TGN as indicated by co-staining with TGN46. In cells transiently overexpressing hPOSH, Gag was almost exclusively at the plasma membrane and could no longer be detected at the TGN suggesting that hPOSH accelerated Gag transport to the plasma membrane. When hPOSH expression was reduced by siRNA treatment, Gag failed to reach the plasma membrane and instead accumulated in punctated intracellular loci segregated from the TGN. Gag was also absent from the plasma membrane in cells overexpressing hPOSH RING mutant (hPOSHC12;52A). However, distinct from the appearance in the siRNA-treated cells, Gag was now perinuclear with a significant portion localizing at the TGN. The difference between the siRNA and the dominant negative effect on Gag intracellular distribution is discussed below.

The ubiquitination activity of RING finger-containing E3 ligases is abrogated by mutations in the zinc-coordinating residues due to inability to recruit E2's. In contrast, RING-finger mutations do not affect substrate binding. Consequently, overexpression of E3 RING mutants often exerts a dominant negative inhibitory effect through futile sequestration of the ubiquitination substrates [Dantuma, 2000 #5]. Thus the arrest of Gag delivery to the plasma membrane caused by overexpression of the RING mutant indicated that hPOSH-mediated ubiquitination was essential for localization of Gag at the plasma membrane and demonstrated for the first time that Gag is sorted to the plasma membrane via the TGN.

### Example 18

### POSH-Regulated Intracellular Transport of Myristoylated Proteins

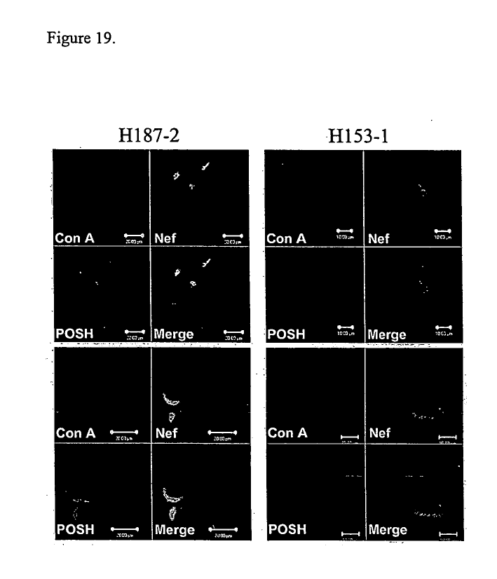

The localization of myristoylated proteins, Gag (see also Example 17 and FIG. 17), HIV-1 Nef, Src and Rapsyn, in cells depleted of hPOSH were analyzed by immunofluorescence. In control cells, HIV-1 Nef was found in a perinuclear region co-localized with hPOSH, indicative of a TGN localization (FIG. 19). When hPOSH expression was reduced by siRNA treatment, Nef expression was weaker relative to control and nef lost its TGN, perinuclear localization. Instead it accumulated in punctated intracellular loci segregated from the TGN.

Src is expressed at the plasma membrane and in intracellular vesicles, which are found close to the plasma membrane (FIG. 20, H187 cells). However, when hPOSH levels were reduced, Src was dispersed in the cytoplasm and loses its plasma membrane proximal localization detected in control (H187) cells (FIG. 20, compare H153-1 and H187-2 panels).

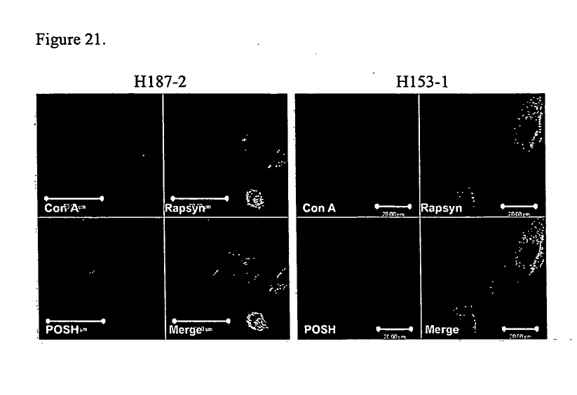

Rapsyn, a peripheral membrane protein expressed in skeletal muscle, plays a critical role in organizing the structure of the nicotinic postsynaptic membrane (Sanes and Lichtman, Annu. Rev. Neurosci. 22: 389-442 (1999)). Newly synthesized Rapsyn associates with the TGN and than transported to the plasma membrane (Marchand et al., J. Neurosci. 22: 8891-01 (2002)). In hPOSH-depleted cells (H1153-1) Rapsyn was dispersed in the cytoplasm, while in control cells it had a punctuated pattern and plasma membrane localization, indicating that hPOSH influences its intracellular transport (FIG. 21).

Materials and Methods Used:

Antibodies:

Src antibody was purchased from Oncogene research products(Darmstadt, Germany). Nef antibodies were pusrchased from ABI (Columbia, Mass.) and Fitzgerald Industries Interantional (Concord, Mass.). Alexa Fluor conjugated antibodies were pusrchased from Molecular Probes Inc. (Eugene, Oreg.).

hPOSH antibody: Glutathione S-transferase (GST) fusion plasmids were constructed by PCR amplification of hPOSH codons 285-430. The amplified PCR products was cloned into pGEX-6P-2 (Amersham Pharmacia Biotech, Buckinghamshire, UK). The truncated hPOSH protein was generated in E. coli BL21. Bacterial cultures were grown in LB media with carbenicillin (100 μg/ml) and recombinant protein production was induced with 1 mM IPTG for 4 hours at 30° C. Cells were lysed by sonication and the recombinant protein was then isolated from the cleared bacterial lysate by affinity chromatography on a glutathione-sepharose resin (Amersham Pharmacia Biotech, Buckinghamshire, UK). The hPOSH portion of the fusion protein was then released by incubation with PreScission protease (Amersham Pharmacia Biotech, Buckinghamshire, UK) according to the manufacturer's instructions and the GST portion was then removed by a second glutathione-sepharose affinity chromatography. The purified partial hPOSH polypeptide was used to immunize New Zealand white rabbits to generate antibody 15B (Washington Biotechnology, Baltimore, Md.).

Construction of siRNA Retroviral Vectors:

hPOSH scrambled oligonucleotide (5′-CACACACTGCCGTCAACTGTTCAA GAGACAGTTGACGGCAGTGTGTGTTTTT-3′ (SEQ ID NO: 126); and 5′-AATTAAAAAACACA CACTGCCGTCAACTGTCTCTTGAACAGTTGACGGCAGTGTGTGGGCC-3′ SEQ ID NO: 127) were annealed and cloned into the ApaI-EcoRI digested pSilencer 1.0-US (Ambion) to generate pSIL-scrambled. Subsequently, the U6-promoter and RNAi sequences were digested with BamHI, the ends filled in and the insert cloned into the Olil site in the retroviral vector, pMSVhyg (Clontech), generating pMSCVhyg-U6-scrambled. hPOSH oligonucleotide encoding RNAi against hPOSH (5′-AACAGAGGCCTTGGAAA CCTGGAAGCTTGCAGGTTTCCAAGGCCTCTGTT-3′ (SEQ ID NO: 128); and 5′-GATCAACAGAGGCCTTGGAAACCTGCAAGCTTCCAGGTTTCCAA GGCCTCTGTT-3′) (SEQ ID NO: 129) were annealed and cloned into the BamHI-EcoRI site of pLIT-U6, generating pLIT-U6 hPOSH-230. pLIT-U6 is an shRNA vector containing the human U6 promoter (amplified by PCR from human genomic DNA with the primers, 5′-GGCCC ACTAGTCAAGGTCGGGCAGGAAGA-3′ (SEQ ID NO: 130) and 5′-GCCGAATT CAAAAAGGATCCGGCGATATCCGGTGTTTCGTCCTTTCCA-3′) (SEQ ID NO: 131) cloned into pLITMUS38 (New England Biolabs) digested with SpeI-EcoRI. Subsequently, the U6 promoter-hPOSH shRNA (pLIT-U6 hPOSH-230 digested with SnaBI and PvuI) was cloned into the Olil site of pMSVhyg (Clontech), generating pMSCVhyg U6-hPOSH-230.

Generation of Stable Clones:

HEK 293T cells were transfected with retroviral RNAi plasmids (pMSCVhyg-U6-Prt3-230 and pMSCVhyg-U6-scrambled and with plasmids encoding VSV-G and moloney gag-pol. Two days post transfection, medium containing retroviruses was collected and filtered and polybrene was added to a final concentration of 8 μg/ml. This was used to infect HeLa SS6 cells grown in 60 mm dishes. Forty-eight hours post-infection cells were selected for RNAi expression by the addition of hygromycin to a final concentration of 300 μg/ml. Clones expressing RNAi against hPOSH were named H153, clones expressing scrambled RNAi were named H187.

Transfection and Immunofluorescent Analysis:

Gag-EGFP experiments are described in Example 17 and FIG. 17.

H153 or H187 cells were transfected with Src or Rapsyn-GFP (Image clone image: 3530551 or pNLenv-1). Eighteen hours post transfection cells were washed with PBS and incubated on ice with Alexa Fluor 647 conjugated Con A to label plasma membrane glycoproteins. Subsequently cells were fixed in 3% paraformaldehyde, blocked with PBS containing 4% bovine serum albumin and 1% gelatin. Staining with rabbit anti-Src, rabbit anti-hPOSH (15B) or mouse anti-nef was followed with secondary antibodies as indicated.

Laser scanning confocal microscopy was performed on LSM510 confocal microscope (Zeiss) equipped with Axiovert 100M inverted microscope using ×40 magnification and 1.3-numerical-aperture oil-immersion lens for imaging. For co-localization experiments, 10 optical horizontal sections with intervals of 1 μm were taken through each preparation (Z-stack). A single median section of each preparation is shown.

### Example 19

### POSH Reduction by siRNA Abrogates West Nile Virus (“WNV”) Infectivity

HeLa SS6 cells were tarnsfected with either control or POSH-specific siRNA. Cells were subsequently infected with WNV (4∴104 PFU/well). Viruses were harvested 24 hours and 48 hours post-infection, serially diluted, and used to infect Vero cells. As a control WNV (4×104 PFU/well), that was not passed through HeLa SS6 cells, was used to infect Vero cells. Virus titer was determined by plaque assay in Vero cells.

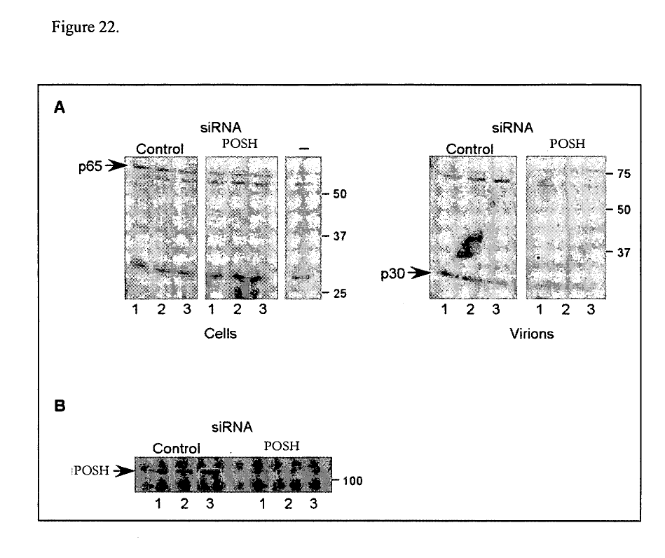

Virus titer was reduced by 2.5-log in cells treated with POSH-specific siRNA relative to cells transfected with control siRNA, thereby indicating that WNV requires POSH for virus secretion. See FIG. 22.

Experimental Procedure:

Cell Culture, Transfections and Infection:

Hela SS6 cells were grown in Dulbecco's modified Eagle's medium (DMEM) supplemented with 10% heat-inactivated fetal calf serum and 100 units/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (100 nM) using lipofectamin 2000 (Invitrogen, Paisley, UK). On the day following the initial transfection, cells were split 1:3 in complete medium and transfected with a second portion of double-stranded siRNA (50 nM). Six hours post-transfection medium was replaced and cells infected with WNv (4×104 PFU/well). Medium was collected from infected HeLa SS6 cells twenty-four and forty-eight post-infection (200 μl), serially diluted, and used to infect Vero cells. Virus titer was determined by plaque assay (Ben-Nathan D, Lachmi B, Lustig S, Feuerstien G (1991) Protection of dehydroepiandrosterone (DHEA) in mice ifected with viral encephalitis. Arch Viro; 120, 263-271).

### Example 20

### HERPUD1 Depletion by siRNA Reduces HIV Maturation

Hela SS6 cells were transfeted with siRNA directed against HERPUD1 and with a plsmid encoding HIV proviral genome (pNLenv-1). Twenty four hours post-HIV transfection, virus-like particles (VLP) secreted into the medium were isolated and reverse transcriptase activity was determined. HIV release of active RT is an indication for a release of processed and mature virus. When the levels of HERPUD1 were reduced RT activity was inhibited by 80%, demonstrating the importance of HERPUDI in HIV-maturation.

Experimental Outline

Cell Culture and Transfection:

HeLa SS6 were kindly provided by Dr. Thomas Tuschl (the laboratory of RNA Molecular Biology, Rockefeller University, New York, N.Y.). Cells were grown in Dulbecco's modified Eagle's medium (DMEM) supplemented with 10% heat-inactivated fetal calf serum and 100 U/ml penicillin and 100 μg/ml streptomycin. For transfections, HeLa SS6 cells were grown to 50% confluency in DMEM containing 10% FCS without antibiotics. Cells were then transfected with the relevant double-stranded siRNA (50-100 nM) (HERPUD1: 5′-GGGAAGUUCUUCGGAACCUdTdT-3′ (SEQ ID NO: 132) and 5′-dTdTCCCUUCAAGAAGCCUUGGA-5′) (SEQ ID NO: 133) using lipofectamin 2000 (Invitrogen, Paisley, UK). A day following the initial transfection cells were split 1:3 in complete medium and co-transfected 24 hours later with HIV-1NLenv1 (2 μg per 6-well) (Schubert et al., J. Virol. 72:2280-88 (1998)) and a second portion of double-stranded siRNA.

Assay for Virus Release

Virus and virus-like particle (VLP) release was determined one day after transfection with the proviral DNA as previously described (Adachi et al., J. Virol. 59: 284-91 (1986); Fukumori et al., Vpr. Microbes Infect. 2: 1011-17 (2000); Lenardo et al., J. Virol. 76: 5082-93 (2002)). The culture medium of virus-expressing cells was collected and centrifuged at 500×g for 10 minutes. The resulting supernatant was passed through a 0.45 μm-pore filter and the filtrate was centrifuged at 14,000×g for 2 hours at 4° C. The resulting supernatant was removed and the viral-pellet was re-suspended in SDS-PAGE sample buffer. The corresponding cells were washed three times with phosphate-buffered saline (PBS) and then solubilized by incubation on ice for 15 minutes in lysis buffer containing the following components: 50 mM HEPES-NaOH, (pH 7.5), 150 mM NaCl, 1.5 mM MgCl₂, 0.5% NP-40, 0.5% sodium deoxycholate, 1 mM EDTA, 1 mM EGTA and 1:200 dilution of protease inhibitor cocktail (Calbiochem, La Jolla, Calif.). The cell detergent extract was then centrifuged for 15 minutes at 14,000×g at 4° C. The VLP sample and a sample of the cleared extract (normally 1:10 of the initial sample) were resolved on a 12.5% SDS-polyacrylamide gel, then transferred onto nitrocellulose paper and subjected to immunoblot analysis with rabbit anti-CA antibodies. The CA was detected either after incubation with a secondary anti-rabbit horseradish peroxidase-conjugated antibody and detected by Enhanced Chemi-Luminescence (ECL) (Amersham Pharmacia) or after incubation with a secondary anti-rabbit antibody conjugated to CyS (Jackson Laboratories, West Grove, Pennsylvania) and detected by fluorescence imaging (Typhoon instrument, Molecular Dynamics, Sunnyvale, Calif.). The Pr55 and CA were then quantified by densitometry and the amount of released VLP was then determined by calculating the ratio between VLP-associated CA and intracellular CA and Pr55 as previously described (Schubert et al., J. Virol. 72:2280-88 (1998)).

Analysis of Reverse Transcriptase Activity in Supernatants

RT activity was determined in pelleted VLP (see above) by using an RT assay kit (Roche, Germany; Cat.No. 1468120). Briefly, VLP pellets were resuspended in 40 μl RT assay lysis buffer and incubated at room temperature for 30 minutes. At the end of incubation 20 μl RT assay reaction mix was added to each sample and incubation continued at 37° C. overnight. Samples (60 μl) were than transferred to MTP strip wells and incubated at 37° C. for 1 hour. Wells were washed five times with wash buffer and DIG-POD added for a one-hour incubation at 37° C. At the end of incubation wells were washed five times with wash buffer and ABST substrate solution was added and incubated until color developed. The absorbance was read in an ELISA reader at 405 nm (reference wavelength 492 nm). The resulting signal intensity is directly proportional to RT activity; RT concentration was determined by plotting against a known amount of RT enzyme included in separate wells of the reaction.

### Example 21

### MSTP028 Reduction by siRNA Decreases HIV VLP Production

This example demonstrates the effects of an siRNA-mediated decrease in MSTP028 expression on the production of HIV virus-like particles in HeLa cells. The effects were measured at steady state.

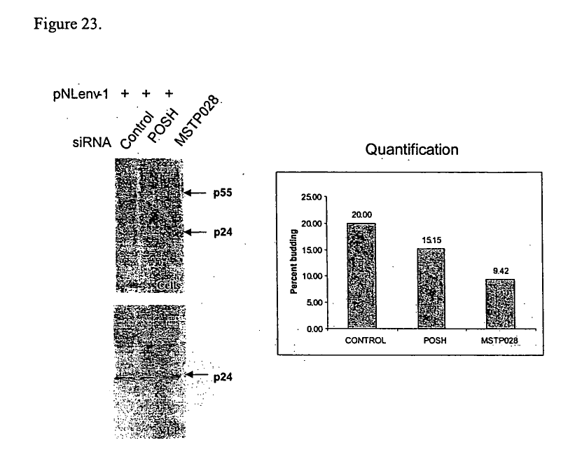

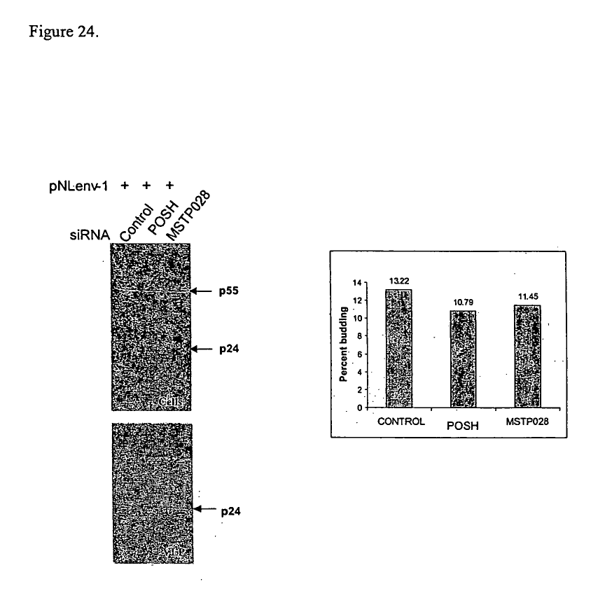

Experiments were performed according to two different protocols. Experiment 1 proceeded with a second transfection on day 3, while Experiment 2 involved an additional exchange of medium on day 3, and proceeded to the second transfection on day 4. The results from Experiment 1 are shown FIG. 23, and those for Experiment 2 are shown in FIG. 24.

Day 1: Preparing Cells

4.5×105 HeLa SS6 cells/well, were seeded in 1×6 well plates. Cells were seeded in transfection medium (growing medium free of antibiotics).

Materials:

| Cat. No. | Manufacture | Reagent Name |
|---|---|---|
| D5796 | Sigma | DMEM |
| 04-121-1A | Beit Haemek | FCS |
| D8537 | Sigma | PBS |
| P4333 | Sigma | Pen/Strep |
| T4049 | Sigma | 0.25% Trypsin-EDTA |

Day 2: Transfection

Materials:

| Cat. No. | Manufacture | Reagent Name |
|---|---|---|
| 11668-027 | Invitrogen | LF2000 reagent |
| 31985-047 | GibcoBRL | OptiMEM |

Experimental and Control Conditions:

1-Control siRNA + pNLEnv-1
2-POSH siRNA + pNLenv-1
3-MSTP028 siRNA + pNLenv-1

1. Two hours before transfection, replace cell media to 2 ml/well complete DMEM without antibiotics.
2. siRNA dilution: for each transfection dilute 100 nm siRNA in 0.25 ml OptiMEM per well.
3. LF 2000 dilution: for each well dilute 5 μl lipofectamine reagent in 0.25 ml OptiMEM.
4. Incubate diluted siRNAs and LF 2000 for 5 minutes at RT.
5. Mix the diluted siRNAs with diluted LF2000 and incubated for 25 minutes at RT.
6. Add the mixture to the cells, 0.5 ml/well (drop wise) and incubate for 24 hours at 37° C. in CO₂ incubator.

Transfections: for each well

(12.5 μl (siRNA)/0.25 ml OptiMEM)×3

LF 2000 35 μl/1.75 ml

Day 3:

Exp. 1: second transfection (as Day 4 below).

Exp. 2: Exchange medium.

Day 4:

Exp. 1: VLP assay (see below).

Exp. 2: Second transfection
1. Two hours before transfection, replace cell media to 2 ml/well complete DMEM without antibiotics.
2. siRNA and DNA dilution: Prepare dilution of plasmid pNLenv-10.75 μg/well in 0.25 ml OptiMEM (total of 3 wells). Divide plasmid dilution to eppendorf tubes (0.25 ml each). To each tube add siRNA 40 nM (2.5 μl).
3. LF 2000 dilution: for each well dilute 5 μl lipofectamine reagent in 0.25 ml OptiMEM.
4. Incubate diluted siRNAs and LF 2000 for 5 minutes at RT.
5. Mix the diluted siRNAs with diluted LF2000 and incubated for 1 hour at RT.
6. Add the mixture to the cells, 0.5 ml/well (drop wise) and incubate for 24 hours at 37° C. in CO₂ incubator.

Day 5:

Exp. 2: VLP assay.

Solutions:

Lysis buffer

| Tris-HCl pH 7.6 | 50 | mM |
|---|---|---|
| MgCl2 | 1.5 | mM |
| NaCl | 150 | mM |
| Glycerol | 10% | |
| NP-40 | 0.5% | |
| DOC | 0.5% | |
| EDTA | 1 | mM |
| EGTA | 1 | mM |

Add PI₃C 1:200.

Steady State VLP Assay

A. Cell Extracts
1. Pellet floating cells by centrifugation (Imin, 14000 rpm at 40C), save supernatant (continue with supernatant immediately to step B), scrape cells in ice-cold PBS, add to the corresponding floated cell pellet and centrifuge for Smin 1800 rpm at 40C.
2. Wash cell pellet once with ice-cold PBS.
3. Resuspend cell pellet (from 6 well) in 100 μl NP40-DOC lysis buffer and incubate 10 minutes on ice.
4. Centrifuge at 14,000 rpm for 15 min. Transfer supernatant to a clean eppendorf.
5. Prepare samples for SDS-PAGE by adding them sample buffer and boil for 10 min—take the same volume for each reaction (15 μl).

B. Purification of VLP from Cell Media
1. Filtrate the supernatant through a 0.45 μm filter.
2. Centrifuge supernatant at 14,000 rpm at 40C for at least 2 h.
3. Resuspend VLP pellet in 50 μl 1× sample buffer and boil for 10 min. Load 25 μl of each sample.

C. Western Blot Analysis
1. Run all samples from stages A and B on Tris-Gly SDS-PAGE 12.5%.
2. Transfer samples to nitrocellulose membrane (100V for 1.15 h.).
3. Dye membrane with ponceau solution.
4. Block with 10% low fat milk in TBS-t for 1 h.
5. Incubate with anti p24 rabbit 1:500 in TBS-t 2 hour (room temperature)—overnight (40C).
6. Wash 3 times with TBS-t for 7 min each wash.
7. Incubate with secondary antibody anti rabbit cy5 1:500 for 30 min.
8. Wash five times for 10 min in TBS-t
9. View in Typhoon for fluorescence signal (650).

### Example 22

### POSH Protein-Protein Interactions

POSH-associated proteins were identified by using a yeast two-hybrid assay.

Procedure:

Bait plasmid (GAL4-BD) was transformed into yeast strain AH109 (Clontech) and transformants were selected on defined media lacking tryptophan. Yeast strain Y187 containing pre-transformed Hela cDNA prey(GAL4-AD) library (Clontech) was mated, according to the Clontech protocol, with a yeast strain containing the bait vector, and plated on defined media lacking tryptophan, leucine, histidine and containing 5 mM 3-amino-triazol. Colonies that grew on the selective media were tested for beta-galactosidase activity and positive clones were further characterized. Plasmid was recovered from yeast colonies and transformed into E. coli DH5alpha strain. After ampicillin selection plasmid was prepared from bacterial colonies and transformed back into yeast strain AH109 together with bait plasmid or empty bait vector and colonies selected on defined media lacking leucine and tryptophan and then scored for growth on media lacking tryptophan, leucine, histidine and containing 5 mM 3-amino-triazol. Only prey clones which their growth on this media was dependent on bait plasmid were scored as true hits. Prey clones were identified by amplifying cDNA insert and sequencing using vector derived primers.

Bait:

Plasmid vector: pGBK-T7 (Clontech)

Plasmid name: pPL269-pGBK-T7 GAL4 POSHdR

Protein sequence: Corresponds to aa 53-888 of POSH (RING domain deleted)

(SEQ ID NO: 134)

RTLVGSGVEELPSNILLVRLLDGIKQRPWKPGPGGGSGTNCTNALRSQSS
TVANCSSKDLQSSQGGQQPRVQSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI
Library screened: Hela pretransformed library
(Clontech).

The POSH-AP, HERPUD1 (Hs.146393), was identified. The POSH-APs, GOCAP1 and Cbl-b, were also identified and are shown in the table below:

TABLE 7
POSH-APs Identified by Yeast Two-Hybrid Screen

| Clone | BLAST hit | UniGene | Name | Remarks |
|---|---|---|---|---|
| 3Gd111 | AK025520 | Hs.6831 | GOCAP1 golgi | |
| | | | complex associated | |
| | | | protein 1, 60 kDa | |
| 3Gd114 | AK094184 | Hs.381921 | Homo sapiens cDNA | 1 seq file in |
| | | | FLJ36865 fis, clone | Unigene |
| | | | ASTRO2016148, | |
| | | | highly similar to | |
| | | | Signal transduction | |
| | | | protein CBL-B | |
| | BC032851 | Hs.3144 | CBLB Cas-Br-M | aa631- |
| | | | (murine) ecotropic | COOH |
| | | | retroviral transforming | |
| | | | sequence b | |

Proteins implicated in TGN transport identified by yeast two-hybrid screening also include: PRKAR1A, Hs.280342; GOSR2, Hs.432552; DDEF1, Hs.386779; ARHV (CHP), Hs. 163834; SPG20 (spartin), Hs.205088; CENTB1, Hs.337242.

Examples of sequences for each of the POSH-APs, HERPUD1, GOCAP1 and Cbl-b, are presented below.

Human HERPUD1 mRNA sequence - var1
(public gi: 16507801)

(SEQ ID NO: 37)

AGAGACGTGAACGGTCGTTGCAGAGATTGCGGGCGGCTGAGACGCCGCCT
GCCTGGCACCTAGGAGCGCAGCGGAGCCCCGACACCGCCGCCGCCGCCAT
GGAGTCCGAGACCGAACCCGAGCCCGTCACGCTCCTGGTGAAGAGCCCCA
ACCAGCGCCACCGCGACTTGGAGCTGAGTGGCGACCGCGGCTGGAGTGTG
GGCCACCTCAAGGCCCACCTGAGCCGCGTCTACCCCGAGCGTCCGCGTCC
AGAGGACCAGAGGTTAATTTATTCTGGGAAGCTGTTGTTGGATCACCAAT
GTCTCACGGACTTGCTTCCAAAGGAAAAACGGCATGTTTTGCATCTCGTG
TGCAATGTGAAGAGTCCTTCAAAAATGCCAGAAATCAACGCCAAGGTGGC
TGAATCCACAGAGGAGCCTGCTGGTTCTAATCGGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCATCACGTTGGGTGGTTTCCATT
TAGACCGAGGCCGGTTCAGAACTTCCCAAATCATGGTCCTCCTCCTGACG
TTGTAAATCAGGACCCCAACAATAACTTACAGGAAGGCACTGATCCTGAA
ACTGAAGACCCCAACCACCTCCCTCCAGACAGGGATGTACTAGATGGCGA
GCAGACCAGCCCCTCCTTTATGAGCACAGCATGGCTTGTCTTCAAGACTT
TCTTTGCCTCTCTTCTTCCAGAAGGCCCCCCAGCCATCGCAAACTGATGG
TGTTTGTGCTGTAGCTGTTGGAGGCTTTGACAGGAATGGACTGGATCACC
TGACTCCAGCTAGATTGCCTCTCCTGGACATGGCAATGATGAGTTTTTAA
AAAACAGTGTGGATGATGATATGCTTTTGTGAGCAAGCAAAAGCAGAAAC
GTGAAGCCGTGATACAAATTGGTGAACAAAAAATGCCCAAGGCTTCTCAT
GTCTTTATTCTGAAGAGCTTTAATATATACTCTATGTAGTTTAATAAGCA
CTGTACGTAGAAGGCCTTAGGTGTTGCATGTCTATGCTTGAGGAACTTTT
CCAAATGTGTGTGTCTGCATGTGTGTTTGTACATAGAAGTCATAGATGCA
GAAGTGGTTCTGCTGGTACGATTTGATTCCTGTTGTTATGTTTAAATTAC
ACTAAGTGTACTACTTTATATAATCAATGAAATTGCTAGACATGTTTTAG
CAGGACTTTTCTAGGAAAGACTTATGTATAATTGCTTTTTAAAATGCAGT
GCTTTACTTTAAACTAAGGGGAACTTTGCGGAGGTGAAAACCTTTGCTGG
GTTTTCTGTTCAATAAAGTTTTACTATGATGACCCTGAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AA
Human HERPUD1 mRNA sequence - var2
(public gi: 10441910)

(SEQ ID NO: 38)

GCTGTGTGGCCCAGGCTTTTCTCAAACTCCTGAGGGCAAGCGATCCTCCC
ACCTCAGCCTCCTGAGTAGCTGGGACTACAGGCATGTGCCACTAGACCTG
GCTCTAAAGACATATATGACACACGAAACCATTTATTTTTCATTTCACAA
TGTTTATTCACATATATGGTATTAGTATTCTAATGTAGTGATGCACTCTA
AATTTGCATTATATTTCCTAGAACATCTGAACAGAGCATAGGAAATTCCC
TATTTTGCCATTATCAGTTCTAACAAAAATCTTAAAAGCACTTTATCATT
TCATTTCCCTGCACTGTAATTTTTTTAAATGATCAAAAACAGTATCATAC
CAAGGCTTACTTATATTGGAATACTATTTTAGAAAGTTGTGGGCTGGGTT
GTATTTATAAATCTTGTTGGTCAGATGTCTGCAATGAGTAAATTTAGCAC
CATTATCACGAAGCTTTCTCACCAATGACAACTTCATTGGAAGATTTTAA
TGAAAGTGTAGCATACTCTAGGGAAAAAATATGAATATTTTAGCATCTAT
GTATTGAAAATTATGTTGAATAAATGTCAGACTATTTTTTACATAACGTT
GCTTCTGTTTAATTTTGTCACGTTCAGAGGTGGGGGGTAGGAGATGTAAG
CCCTTGACACCAAAATAATTCCTTTTGCTTGATTTCAGACAGTTGCATCA
GCTCCTTTGTTCTGTGTTCATGTTACACTTATTTAGGTGGCTGAATCCAC
AGAGGAGCCTGCTGGTTCTAATCGGGGACAGTATCCTGAGGATTCCTCAT
GTGATGGTTTAAGGCAAAGGGAAGTTCTTCGGAACCTTTCTTCCCCTGGA
TGGGAAAACATCTCAAGGCCTGAAGCTGCCCACCAGGCATTCCAAGGCCT
GGGTCCTGGTTTCTCCGGTTACACACCCTATGGGTGGCTTCAGCTTTCCT
GGTTCCAGCAGATATATGCACGACAGTACTACATGCAATATTTAGCAGCC
ACTGCTGCATCAGGGGCTTTTGTTCCACCACCAAGTGCACAAGAGATACC
TGTGGTCTCTGCACCTGCTCCAGCCCCTATTCACAACCAGTTTCCAGCTG
AAAACCAGCCTGCCAATCAGAATGCTGCTCCTCAAGTGGTTGTTAATCCT
GGAGCCAATCAAAATTTGCGGATGAATGCACAAGGTGGCCCTATTGTGGA
AGAAGATGATGAAATAAATCGAGATTGGTTGGATTGGACCTATTCAGCAG
CTACATTTTCTGTTTTTCTCAGTATCCTCTACTTCTACTCCTCCCTGAGC
AGATTCCTCATGGTCATCGGGGCCACCGTTGTTATGTACCTGCATCACGT
TGGGTGGTTTCCATTTAGACCGAGGCCGGTTCAGAACTTCCCAAATGATG
GTCCTCCTCCTGACGTTGTAAATCAGGACCCCAACAATAACTTACAGGAA
GGCACTGATCCTGAAACTGAAGACCCCAACCACCTCCCTCCAGACAGGGA
TGTACTAGATGGCGAGCAGACCAGCCCCTCCTTTATGAGCACAGCATGGC
TTGTCTTCAAGACTTTCTTTGCCTCTCTTCTTCCAGAAGGCCCCCCAGCC
ATCGCAAACTGATGGTGTTTGTGCTGTAGCTGTTGGAGGCTTTGACAGGA
ATGGACTGGATCACCTGACTCCAGCTAGATTGCCTCTCCTGGACATGGCA
ATGATGAGTTTTTAAAAAACAGTGTGGATGATATGATGCTTTTGTGAGCA
AGCAAAAGCAGAAACGTGAAGCCGTGATACAAATTGGTGAACAAAAAATG
CCCAAGGCTTCTCATGTCTTTATTCTGAAGAGCTTTAATATATACTCTAT
GTAGTTTAATAAGCACTGTACGTAGAAGGCCTTAGGTCTTGCATGTCTAT
GCTTGAGGAACTTTTCCAAATGTGTGTGTCTGCATGTGTGTTTGTACATA
GAAGTCATAGATGCAGAAGTGGTTCTGCTGGTACGATTTGATTCCTGTTG
GAATGTTTAAATTACACTAAGTGTACTACTTTATATAATCAATCAAATTG
CTAGACATGTTTTAGCAGGACTTTTCTAGGAAAGACTTATGTATAATTGC
TTTTTAAAATGCAGTGCTTTACTTTAAACTAAGGGGAACTTTGCGGAGGT
GAAAACCTTTGCTGGGTTTTCTGTTCATAAGTTTTACTATGAATGAAAAA
AAAAAAAAAAAAAAAAA
Human HERPUD1 mRNA sequence - var3
(public gi: 3005722)

(SEQ ID NO: 39)

GGCCACCTCAAGGCCCACCTGAGCCGCGTCTACCCCGAGCGTCCGCGTCC
AGACGACCAGAGGTTAATTTATTCTGGGAAGCTGTTGTTGGATCACCAAT
GTCTCAGGGACTTGCTTCCAAAGGAAAAACCGCATGTTTTGCATCTGGTG
TGCAATGTGAAGAGTCCTTCAAAAATGCCAGAAATCAACGCCAAGGTCGC
TGAATCCACAGAGGAGCCTGCTGGTTCTAATCGGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCAGATATATGCACGACAGTACTACATGCAATAT
TTAGCAGCCACTGCTGCATCAGGGGCTTTTGTTCCACCACCAAGTGCACA
AGAGATACCTGTGGTCTCTCCACCTGCTCCAGCCCCTATTCACAACCAGT
TTCCAGCTGAAAACCAGCCTGCCAATCAGAATGCTGCTCCTCAAGTCGTT
GTTAATCCTGGAGCCAATCAAAATTTGCGGATGAATGCACAACGTGGCCC
TATTGTGGAAGAAGATGATGAAATAAATCGAGATTGGTTGGATTGGACCT
ATTCAGCAGCTACATTTTCTGTTTTTCTCAGTATCCTCTACTTCTACTCC
TCCCTGAGCAGATTCCTCATGGTCATGGGGGCCACCGTTGTTATGTACCT
GCATCACGTTGGGTGGTTTCCATTTAGACCGAGGCCGGTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCACCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATCAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTTATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAAACCTTTGCTGGGTTTTCTGTTCAATAAAGTTTTACTA
TGAATGACCCTGAAAAAAAAAAAAAAAAAAAAAA
Human HERPUD1 mRNA sequence - var4
(public gi: 21619176)

(SEQ ID NO: 40)

CCACGCGTCCGGGTCGTTGCAGAGATTGCGGGCGGCTGAGACGCCGCCTG
CCTGGCACCTAGGAGCGCAGCGGAGCCCCGACACCGCCGCCGCCGCCATG
GAGTCCGAGACCGAACCCGAGCCCGTCACGCTCCTGGTGAAGAGCCCCAA
CCAGCGCCACCGCGACTTGGAGCTGAGTGGCGACCGCGGCTGGAGTGTGG
GCCACCTCAAGGCCCACCTGAGCCGCGTCTACCCCGAGCGTCCGCGTCCA
GAGGACCAGAGGTTAATTTATTCTGGGAAGCTGTTGTTGGATCACCAATG
TCTCAGGGACTTGCTTCCAAAGCACGAAAAACGGCATGTTTTGCATCTGG
TGTGCAATGTGAAGAGTCCTTCAAAAATGCCAGAAATCAACGCCAACGTG
GCTGAATCCACAGAGGAGCCTGCTGGTTCTAATCGGGGACAGTATCCTGA
GGATTCCTCAAGTGATCGTTTAAGGCAAAGGGAAGTTCTTCGGAACCTTT
CTTCCCCTGGATGGGAAAACATCTCAAGGCCTGAAGCTGCCCAGCAGGCA
TTCCAAGGCCTGGGTCCTGGTTTCTCCGGTTACACACCCTATGGGTGGCT
TCAGCTTTCCTGGTTCCAGCAGATATATGCACGACAGTACTACATGCAAT
ATTTAGCAGCCACTGCTGCATCAGGGGCTTTTGTTCCACCACCAAGTGCA
CAAGAGATACCTGTGGTCTCTGCACCTGCTCCACCCCCTATTCACAACCA
GTTTCCAGCTGAAAACCAGCCTGCCAATCAGAATGCTGCTCCTCAAGTGG
TTGTTAATCCTGGAGCCAATCAAAATTTGCGGATGAATGCACAAGGTGGC
CCTATTGTGGAAGAAGATGATGAAATAAATCGAGATTGGTTGGATTGGAC
CTATTCAGCAGCTACATTTTCTGTTTTTCTCAGTATCCTCTACTTCTACT
CCTCCCTGAGCAGATTCCTCATGGTCATGGGGGCCACCGTTGTTATGTAC
CTGCATCACGTTGGGTGGTTTCCATTTAGACCGAGGCCGGTTCAGAACTT
CCCAAATGATGGTCCTCCTCCTGACGTTGTAAATCAGGACCCCAACAATA
ACTTACAGGAAGGCACTGATCCTGAAACTGAAGACCCCAACCACCTCCCT
CCAGACAGGGATGTACTAGATCGCGAGCAGACCAGCCCCTCCTTTATGAG
CACAGCATGGCTTGTCTTCAAGACTTTCTTTGCCTCTCTTCTTCCAGAAG
GCCCCCCAGCCATCGCAAACTGATGGTGTTTGTGCTGTAGCTGTTGGAGG
CTTTGACAGGAATGGACTCGATCACCTGACTCCAGCTAGATTGCCTCTCC
TGGACATGGCAATGATGAGTTTTTAAAAAACAGTGTGGATGATGATATGC
TTTTCTGAGCAAGCAAAGCAGAAACGTGAAGCCGTGATACAAATTGGTGA
ACAAAAAATGCCCAAGGCTTCTCATGTCTTTATTCTGAAGAGCTTTAATA
TATACTCTATGTAGTTTAATAAGCACTGTACGTAGAAGGCCTTAGGTCTT
GCATGTCTATGCTTGAGGAACTTTTCCAAATGTGTGTGTCTGCATGTGTG
TTTGTACATAGAAGTCATAGATGCAGAAGTGGTTCTGCTGGTACGATTTG
ATTCCTGTTGGAATGTTTAAATTACACTAAGTGTACTACTTTATATAATC
AATGAAATTGCTAGACATGTTTTAGCAGGACTTTTCTAGGAAAGACTTAT
GTATAATTGCTTTTTAAAATGCAGTGCTTTACTTTAAACTAAGGGGAACT
TTCCGGAGGTGAAAACCTTTGCTGGGTTTTCTGTTCAATAAAGTTTTACT
ATGAATGACCCTGAAAAAAAAAAAAAAA
Human HERPUD1 mRNA sequence - var5
(public gi: 14249882)

(SEQ ID NO: 41)

AACGGTCCTTCCAGAGATTGCGGGCCGCTGAGACGCCGCCTGCCTGGCAC
CTAGGAGCGCAGCGGAGCCCCGACACCGCCGCCGCCGCCATGGAGTCCGA
GACCGAACCCGAGCCCGTCACGCTCCTGGTGAAGAGCCCCAACCAGCGCC
ACCGCGACTTGGAGCTGAGTGGCGACCGCCGCTCGAGTCTGGGCCACCTC
AAGGCCCACCTGAGCCGCGTCTACCCCGAGCGTCCGCGTCCAGACGACCA
GAGGTTAATTTATTCTGGGAAGCTGTTGTTGGATCACCAATGTCTCAGGG
ACTTGCTTCCAAAGCACGAAAAACGGCATGTTTTGCATCTGGTGTGCAAT
GTGAAGAGTCCTTCAAAAATGCCAGAAATCAACGCCAAGGTGGCTGAATC
CACAGAGGAGCCTGCTGGTTCTAATCGGGGACAGTATCCTGAGGATTCCT
CAAGTGATGGTTTAAGGCAAAGGGAAGTTCTTCGGAACCTTTCTTCCCCT
GGATGGGAAAACATCTCAAGGCCTGAAGCTGCCCACCAGGCATTCCAAGG
CCTGGGTCCTGGTTTCTCCGGTTACACACCCTATGGGTGGCTTCAGCTTT
CCTGGTTCCAGCAGATATATGCACGACAGTACTACATGCAATATTTAGCA
GCCACTGCTGCATCAGGGGCTTTTGTTCCACCACCAAGTGCACAAGAGAT
ACCTGTCGTCTCTGCACCTGCTCCAGCCCCTATTCACAACCAGTTTCCAG
CTGAAAACCAGCCTGCCAATCAGAATGCTGCTCCTCAAGTGGTTGTTAAT
CCTGGAGCCAATCAAAATTTGCGGATGAATGCACAAGGTGGCCCTATTGT
GGAAGAAGATGATGAAATAAATCGAGATTGGTTGGATTGGACCTATTCAG
CAGCTACATTTTCTGTTTTTCTCAGTATCCTCTACTTCTACTCCTCCCTG
AGCAGATTCCTCATGGTCATGGGGGCCACCGTTGTTATGTACCTGCATCA
CGTTGGGTGGTTTCCATTTAGACCGAGGCCGGTTCAGAACTTCCCAAATG
ATGGTCCTCCTCCTGACGTTGTAAATCAGGACCCCAACAATAACTTACAG
GAAGGCACTGATCCTGAAACTGAAGACCCCAACCACCTCCCTCCAGACAG
GGATGTACTAGATGGCGAGCAGACCAGCCCCTCCTTTATGAGCACAGCAT
GGCTTGTCTTCAAGACTTTCTTTGCCTCTCTTCTTCCAGAAGGCCCCCCA
GCCATCGCAAACTGATGGTGTTTGTGCTGTAGCTGTTGGAGGCTTTGACA
GGAATGGACTGGATCACCTGACTCCAGCTAGATTGCCTCTCCTGGACATG
GCAATGATGAGTTTTTAAAAAACAGTGTGGATGATGATATGCTTTTGTGA
GCAAGCAAAAGCAGAAACGTGAAGCCGTGATACAAATTGGTGAACAAAAA
ATGCCCAAGGCTTCTCATGTCTTTTATTCTGAAGAGCTTTAATATATACT
CTATGTAGTTAATAAGCACTGTACGTAGAAGGCCTTAGGTGTTGCATGTC
TATGCTTGAGGAACTTTTCCAAATGTGTGTGTCTGCATGTGTGTTTGTAC
ATAGAAGTCATAGATGCAGAAGTGGTTCTGCTGGTACGATTTGATTCCTG
TTGGAATGTTTAAATTACACTAAGTGTACTACTTTATATAATCAATGAAA
TTGCTAGACATGTTTTAGCAGGACTTTTCTACGAAAGACTTATGTATAAT
TGCTTTTTAAAATGCAGTGCTTTACTTTAAACTAAGGGGAACTTTGCGGA
GGTGAAAACCTTTCCTGGGTTTTCTGTTCAATAAAGTTTTACTATGAAAA
AAAAAAAAAAAAAA
Human HERPUD1 mRNA sequence - var6
(public gi: 12652674)

(SEQ ID NO: 42)

GAACTGTCGTTGCAGAGATTGCGGGCGGCTGAGACGCCGCCTGCCTGGCA
CCTAGGAGCGCAGCGGAGCCCCGACACCGCCGCCGCCGCCATGGAGTCCG
AGACCCAACCCGAGCCCGTCACGCTCCTGGTGAAGAGCCCCAACCAGCGC
CACCGCGACTTGGAGCTGAGTGGCGACCGCGGCTGGAGTGTGGGCCACCT
CAAGGCCCACCTGAGCCCCGTCTACCCCGAGCGTCCGCGTCCAGAGGACC
AGAGGTTAATTTATTCTGGGAAGCTGTTGTTCGATCACCAATGTCTCAGG
GACTTGCTTCCAAAGCAGGAAAAACGGCATGTTTTGCATCTGGTGTGCAA
TGTGAAGAGTCCTTCAAAAATGCCAGAAATCAACGCCAAGGTGGCTGAAT
CCACAGAGGAGCCTGCTGGTTCTAATCGGCGACAGTATCCTGAGGATTCC
TCAAGTGATGGTTTAAGGCAAACGGAAGTTCTTCGGAACCTTTCTTCCCC
TGGATGGGAAAACATCTCAAGGCCTGAAGCTGCCCAGCAGGCATTCCAAG
GCCTGGGTCCTGGTTTCTCCGGTTACACACCCTATGGGTGGCTTCAGCTT
TCCTGGTTCCAGCAGATATATGCACGACAGTACTACATGCAATATTTAGC
AGCCACTGCTGCATCAGGGGCTTTTGTTCCACCACCAAGTGCACAAGAGA
TACCTGTGGTCTCTGCACCTGCTCCAGCCCCTATTCACAACCAGTTTCCA
GCTGAAAACCAGCCTGCCAATCAGAATGCTGCTCCTCAAGTGGTTGTTAA
TCCTGGAGCCAATCAAAATTTGCGGATGAATGCACAAGGTGGCCCTATTG
TGGAAGAAGATGATGAAATAAATCGAGATTGGTTGGATTGGACCTATTCA
GCAGCTACATTTTCTGTTTTTCTCAGTATCCTCTACTTCTACTCCTCCCT
GAGCAGATTCCTCATGGTCATGGGGGCCACCGTTGTTATGTACCTGCATC
ACGTTGGGTGGTTTCCATTTAGACCGAGGCCGGTTCAGAACTTCCCAAAT
GATGGTCCTCCTCCTGACGTTGTAAATCAGGACCCCAACAATAACTTACA
GGAAGGCACTGATCCTGAAACTGAAGACCCCAACCACCTCCCTCCAGACA
GGGATGTACTAGATGGCGAGCAGACCAGCCCCTCCTTTATGAGCACAGCA
TGGCTTGTCTTCAAGACTTTCTTTGCCTCTCTTCTTCCAGAAGGCCCCCC
AGCCATCGCAAACTGATGGTGTTTGTGCTGTAGCTGTTGGAGGCTTTGAC
AGGAATGGACTGGATCACCTGACTCCAGCTAGATTGCCTCTCCTGGACAT
GGCAATGATGAGTTTTTAAAAAACAGTGTCGATGATGATATGCTTTTGTG
AGCAAGCAAAAGCAGAAACGTGAAGCCGTGATACAAATTGGTGAACAAAA
AATGCCCAAGGCTTCTCATGTCTTTATTCTGAAGAGCTTTAATATATACT
CTATGTAGTTTAATAAGCACTGTACGTAGAAGGCCTTAGGTGTTGCATGT
CTATGCTTGAGGAACTTTTCCAAATGTGTGTGTCTGCATGTGTGTTTGTA
CATAGAAGTCATAGATGCAGAAGTGGTTCTGCTGGTACGATTTGATTCCT
GTTGGAATGTTTAAATTACACTAAGTCTACTACTTTATATAATCAATGAA
ATTGCTAGACATGTTTTAGCAGGACTTTTCTAGGAAAGACTTATGTATAA
TTGCTTTTTAAAATGCAGTGCTTTACTTTAAACTAAGGGGAACTTTGCGG
AGGTGAAAACCTTTGCTGGGTTTTCTGTTCAATAAAGTTTTACTATGAAT
GAAAAAAAAAAAAAAAAAAAA
Human HERPUD1 mRNA sequence - var7
(public gi: 9711684)

(SEQ ID NO: 43)

AGAGACGTGAACTCTCGTTGCAGAGATTGCGGGCGGCTGAGACGCCGCCT
GCCTGGCACCTAGGAGCGCAGCGGAGCCCCGACACCGCCGCCGCCGCCAT
GGAGTCCGAGACCGAACCCGAGCCCGTCACCCTCCTGGTGAAGAGCCCCA
ACCAGCGCCACCGCCACTTGGAGCTGAGTGGCGACCGCGGCTGGAGTGTG
GGCCACCTCAAGGCCCACCTGAGCCGCGTCTACCCCGAGCGTCCGCGTCC
AGAGGACCAGAGGTTAATTTATTCTGGGAAGCTGTTGTTGGATCACCAAT
GTCTCAGGGACTTGCTTCCAAAGCAGGAAAAACGGCATGTTTTGCATCTC
GTGTGCAATGTGAAGAGTCCTTCAAAAATGCCAGAAATCAACGCCAAGGT
GGCTGAATCCACAGAGGAGCCTGCTGGTTCTAATCGGGGACAGTATCCTG
AGGATTCCTCAAGTGATGGTTTAAGGCAAAGGGAAGTTCTTCGGAACCTT
TCTTCCCCTGGATGGGAAAACATCTCAAGGCCTGAAGCTGCCCAGCAGGC
ATTCCAAGGCCTGGGTCCTGGTTTCTCCGGTTACACACCCTATGGGTGGC
TTCAGCTTTCCTGGTTCCAGCAGATATATGCACGACAGTACTACATGCAA
TATTTAGCAGCCACTGCTGCATCAGGGGCTTTTGTTCCACCACCAAGTGC
ACAAGAGATACCTGTCGTCTCTGCACCTGCTCCAGCCCCTATTCACAACC
AGTTTCCAGCTGAAAACCAGCCTGCCAATCAGAATGCTGCTCCTCAAGTG
GTTGTTAATCCTGGAGCCAATCAAAATTTGCGGATGAATGCACAAGGTGG
CCCTATTGTGGAAGAAGATGATGAAATAAATCGACATTCGTTGGATTGGA
CCTATTCAGCAGCTACATTTTCTCTTTTTCTCAGTATCCTCTACTTCTAC
TCCTCCCTGAGCAGATTCCTCATGGTCATGGGGGCCACCGTTGTTATGTA
CCTGCATCACGTTGGGTGCTTTCCATTTAGACCGAGGCCGGTTCAGAACT
TCCCAAATGATGGTCCTCCTCCTGACGTTGTAAATCAGGACCCCAACAAT
AACTTACAGGAACGCACTGATCCTGAAACTGAAGACCCCAACCACCTCCC
TCCAGACAGGGATGTACTAGATGGCGAGCAGACCAGCCCCTCCTTTATGA
GCACAGCATGGCTTGTCTTCAAGACTTTCTTTGCCTCTCTTCTTCCAGAA
GGCCCCCCAGCCATCGCAAACTGATGGTGTTTGTGCTGTAGCTGTTGGAG
GCTTTGACAGGAATGGACTGGATCACCTGACTCCAGCTAGATTGCCTCTC
CTGGACATGGCAATGATGAGTTTTTAAAAAACAGTGTGGATGATGATATG
CTTTTGTGAGCAAGCAAAAGCAGAAACGTGAAGCCGTGATACAAATTGGT
GAACAAAAAATGCCCAAGGCTTCTCATGTCTTTATTCTGAAGAGCTTTAA
TATATACTCTATGTAGTTTAATAAGCACTGTACGTAGAAGGCCTTAGGTG
TTGCATGTCTATGCTTGACGAACTTTTCCAAATGTGTGTGTCTGCATGTC
TGTTTGTACATAGAAGTCATAGATGCAGAAGTGGTTCTGCTGGTACGATT
TGATTCCTGTTGGAATGTTTAAATTACACTAAGTGTACTACTTTATATAA
TCAATGAAATTGCTAGACATGTTTTAGCAGGACTTTTCTAGGAAAGACTT
ATGTATAATTGCTTTTTAAAATGCAGTGCTTTACTTTAAACTAAGGGGAA
CTTTGCGGAGGTGAAAACCTTTGCTGGGTTTCTGTTCAATAAAGTTTTAC
TATGAATGACCCTG
Human HERPUD1 mRNA sequence - var8
(public gi: 3005718)

(SEQ ID NO: 44)

GACGTGAACGGTCGTTGCAGAGATTGCGGGCGGCTGAGACGCCGCCTGCC
TGGCACCTACGAGCGCAGCGGAGCCCCGACACCGCCGCCGCCGCCATGGA
GTCCGAGACCGAACCCCAGCCCGTCACCCTCCTGGTGAAGAGCCCCAACC
AGCGCCACCGCGACTTGGAGCTGAGTGGCGACCGCGGCTGGAGTGTGGGC
CACCTCAACGCCCACCTGAGCCGCGTCTACCCCGAGCGTCCGCCTCCAGA
GGACCAGAGGTTAATTTATTCTGGGAAGCTGTTCTTCGATCACCAATGTC
TCAGGGACTTGCTTCCAAAGCAGGAAAAACGGCATGTTTTGCATCTGGTG
TGCAATGTGAAGAGTCCTTCAAAAATGCCAGAAATCAACGCCAAGGTGGC
TGAATCCACAGAGGAGCCTGCTGGTTCTAATCCGGGACACTATCCTGAGG
ATTCCTCAAGTGATGGTTTAAGGCAAAGGGAAGTTCTTCGGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCACATATATGCACGACAGTACTACATGCAATAT
TTAGCAGCCACTGCTGCATCAGGGGCTTTTGTTCCACCACCAAGTGCACA
AGAGATACCTGTGGTCTCTGCACCTGCTCCAGCCCCTATTCACAACCAGT
TTCCAGCTGAAAACCAGCCTGCCAATCAGAATGCTGCTCCTCAAGTGGTT
GTTAATCCTGGAGCCAATCAAAATTTGCGGATGAATGCACAAGGTGGCCC
TATTGTGGAAGAAGATGATGAAATAAATCGAGATTGGTTGGATTGGACCT
ATTCAGCAGCTACATTTTCTGTTTTTCTCAGTATCCTCTACTTCTACTCC
TCCCTGAGCAGATTCCTCATGGTCATGGGGGCCACCGTTGTTATGTACCT
GCATCACGTTGGGTGGTTTCCATTTAGACCGAGGCCGCTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCAGCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATGAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTTATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAAACCTTTGCTGGGTTTTCTGTTCAATAAAGTTTTACTA
TGAATGACCCTGAAAAAAAAAAAAAAAAAAAAAA
Human HERPUD1 mRNA sequence - var9
(public gi: 285960)

(SEQ ID NO: 45)

CGTGAACGGTCGTTGCAGAGATTGCGGGCGGCTGAGACGCCGCCTGCCTG
GCACCTAGGAGCGCAGCGGAGCCCCGACACCGCCGCCGCCGCCATGGAGT
CCGAGACCGAACCCGAGCCCGTCACGCTCCTGGTGAAGAGCCCCAACCAG
CGCCACCGCGACTTGGAGCTGAGTGGCGACCGCGGCTGGAGTGTGGGCCA
CCTCAAGGCCCACCTGAGCCGCGTCTACCCCGAGCGTCCGCGTCCAGAGG
ACCAGAGGTTAATTTATTCTGGGAAGCTGTTGTTGGATCACCAATGTCTC
AGGGACTTGCTTCCAAAGCAGGAAAAACGGCATGTTTTGCATCTGCTGTG
CAATGTGAAGAGTCCTTCAAAAATGCCAGAAATCAACGCCAAGGTGGCTG
AATCCACAGAGGAGCCTGCTGGTTCTAATCGGGGACAGTATCCTGAGGAT
TCCTCAAGTGATGGTTTAAGGCAAAGGGAAGTTCTTCGGAACCTTTCTTC
CCCTGGATGGGAAAACATCTCAAGGCCTGAAGCTGCCCAGCAGGCATTCC
AAGGCCTGGGTCCTGGTTTCTCCGGTTACACACCCTATGGGTGGCTTCAG
CTTTCCTGGTTCCACCAGATATATGCACGACAGTACTACATGCAATATTT
AGCAGCCACTGCTGCATCAGGGGCTTTTGTTCCACCACCAAGTGCACAAG
AGATACCTCTGGTCTCTGCACCTGCTCCAGCCCCTATTCACAACCAGTTT
CCAGCTGAAAACCAGCCTGCCAATCAGAATGCTGCTCCTCAAGTGGTTGT
TAATCCTGGAGCCAATCAAAATTTGCGGATGAATGCACAAGGTGGCCCTA
TTGTGGAAGAAGATGATGAAATAAATCGAGATTGGTTGGATTGGACCTAT
TCAGCAGCTACATTTTCTGTTTTTCTCAGTATCCTCTACTTCTACTCCTC
CCTGAGCAGATTCCTCATGGTCATGGGGGCCACCGTTGTTATGTACCTGC
ATCACGTTGGGTGGTTTCCATTTAGACCGAGGCCGGTTCAGAACTTCCCA
AATGATCGTCCTCCTCCTGACGTTGTAAATCAGGACCCCAACAATAACTT
ACACGAAGGCACTGATCCTGAAACTGAAGACCCCAACCACCTCCCTCCAG
ACAGGGATGTACTAGATGGCGAGCAGACCAGCCCCTCCTTTATGAGCACA
GCATGGCTTGTCTTCAAGACTTTCTTTGCCTCTCTTCTTCCAGAAGGCCC
CCCAGCCATCGCAAACTGATGGTGTTTGTGCTGTAGCTGTTGGAGGCTTT
GACAGGAATGGACTGGATCACCTGACTCCAGCTAGATTGCCTCTCCTGGA
CATGGCAATGATGAGTTTTTAAAAAACAGTGTGGATGATGATATGCTTTT
GTGAGCAAGCAAAAGCAGAAACGTGAAGCCGTGATACAAATTGGTGAACA
AAAAATGCCCAAGGCTTCTCATGTGTTTATTCTGAAGAGCTTTAATATAT
ACTCTATGTAGTTTAATAAGCACTGTACCTAGAAGGCCTTAGGTGTTGCA
TGTCTATGCTTGAGGAACTTTTCCAAATGTGTGTGTCTGCATGTGTGTTT
GTACATAGAAGTCATAGATGCAGAAGTGGTTCTGCTGGTAAGATTTGATT
CCTGTTGGAATGTTTAAATTACACTAAGTGTACTACTTTATATAATCAAT
GAAATTGCTAGACATGTTTTAGCAGGACTTTTCTAGGAAAGACTTATGTA
TAATTGCTTTTTAAAATGCAGTGCTTTACTTTAAACTAAGGGGAACTTTG
CGGAGGTGAAAACCTTTGCTGGGTTTTCTGTTCAATAAAGTTTTACTATG
AATGACCCTG
Human HERPUD1 mRNA sequence - var10
(public gi: 7661869)

(SEQ ID NO: 46)

GACGTGAACGGTCGTTGCAGAGATTGCGGGCGGCTGAGACGCCGCCTGCC
TCGCACCTAGGAGCGCAGCGGAGCCCCGACACCGCCGCCGCCGCCATGGA
GTCCGAGACCGAACCCGAGCCCGTCACGCTCCTGGTGAAGAGCCCCAACC
AGCGCCACCGCGACTTGGAGCTGAGTGGCGACCGCGGCTGGAGTCTGCGC
CACCTCAAGGCCCACCTGAGCCGCGTCTACCCCGACCGTCCGCGTCCAGA
GGACCAGAGGTTAATTTATTCTGGGAAGCTGTTGTTGGATCACCAATGTC
TCAGGGACTTGCTTCCAAAGCAGGAAAAACGGCATCTTTTGCATCTGGTG
TGCAATGTGAAGACTCCTTCAAAAATGCCAGAAATCAACGCCAAGGTGGC
TGAATCCACAGAGGAGCCTGCTGGTTCTAATCCGGGACAGTATCCTGAGG
ATTCCTCAAGTGATGGTTTAAGGCAAAGGGAAGTTCTTCGGAACCTTTCT
TCCCCTGGATGGGAAAACATCTCAAGGCCTGAAGCTGCCCAGCAGGCATT
CCAAGGCCTGGGTCCTGGTTTCTCCGGTTACACACCCTATGGGTGGCTTC
AGCTTTCCTGGTTCCAGCAGATATATGCACGACAGTACTACATGCAATAT
TTAGCAGCCACTGCTGCATCAGGGGCTTTTGTTCCACCACCAACTGCACA
AGAGATACCTGTGGTCTCTGCACCTGCTCCAGCCCCTATTCACAACCAGT
TTCCAGCTGAAAACCAGCCTGCCAATCAGAATGCTGCTCCTCAAGTGGTT
GTTAATCCTGGACCCAATCAAAATTTGCGGATGAATGCACAAGGTGGCCC
TATTGTGGAAGAAGATGATGAAATAAATCGAGATTGGTTGGATTGGACCT
ATTCAGCAGCTACATTTTCTGTTTTTCTCAGTATCCTCTACTTCTACTCC
TCCCTGAGCAGATTCCTCATGGTCATCGGGGCCACCGTTGTTATGTACCT
GCATCACGTTGGGTGGTTTCCATTTAGACCGAGGCCGGTTCAGAACTTCC
CAAATGATGGTCCTCCTCCTGACGTTGTAAATCAGGACCCCAACAATAAC
TTACAGGAAGGCACTGATCCTGAAACTGAAGACCCCAACCACCTCCCTCC
AGACAGGGATGTACTAGATGGCGAGCAGACCAGCCCCTCCTTTATGAGCA
CAGCATGGCTTGTCTTCAAGACTTTCTTTGCCTCTCTTCTTCCAGAAGGC
CCCCCAGCCATCGCAAACTGATGGTGTTTGTGCTGTAGCTGTTGGAGGCT
TTGACAGGAATGGACTGGATCACCTGACTCCAGCTAGATTGCCTCTCCTG
GACATGGCAATGATGAGTTTTTAAAAAACAGTGTGGATGATGATATGCTT
TTGTGAGCAAGCAAAAGCAGAAACGTGAAGCCGTGATACAAATTGGTGAA
CAAAAAATGCCCAAGGCTTCTCATGTCTTTATTCTGAAGAGCTTTAATAT
ATACTCTATGTAGTTTAATAAGCACTGTACGTAGAAGGCCTTAGGTGTTG
CATGTCTATGCTTGAGGAACTTTTCCAAATGTGTGTGTCTGCATGTGTGT
TTGTACATAGAAGTCATAGATGCAGAAGTGGTTCTGCTGGTACGATTTGA
TTCCTGTTGGAATGTTTAAATTACACTAAGTGTACTACTTTATATAATCA
ATGAAATTGCTAGACATGTTTTAGCAGGACTTTTCTAGGAAAGACTTATG
TATAATTGCTTTTTAAAATGCAGTGCTTTACTTTAAACTAAGGGGAACTT
TGCGGAGGTGAAAACCTTTGCTGGTTTTCTGTTCAATAAAGTTTTACTAT
GATGACCCTGAAAAAAAAAAAAAAAAAAAAAAAA
Human HERPUD1 Protein sequence - var1
(public gi: 16507802)

(SEQ ID NO: 75)

MESETEPEPVTLLVKSPNQRHRDLELSGDRGWSVGHLKAHLSRVYPERPR
PEDQRLIYSGKLLLDHQCLRDLLPKEKRHVLHLVCNVKSPSKMPEINAKV
AESTEEPAGSNRGQYPEDSSSDGLRQREVLRNLSSPGWENISRHHVGWFP
FRPRPVQNFPNDGPPPDVVNQDPNNNLQEGTDPETEDPNHLPPDRDVLDG
EQTSPSFMSTAWLVFKTFFASLLPEGPPAIAN
Human HERPUD1 Protein sequence - var2
(public gi: 10441911)

(SEQ ID NO: 76)

MQYLAATAASGAFVPPPSAQEIPVVSAPAPAPIHNQFPAENQPANQNAAP
QVVVNPGANQNLRMNAQGGPIVEEDDEINRDWLDWTYSAATFSVFLSILY
FYSSLSRFLMVMGATVVMYLHHVGWFPFRPRPVQNFPNDGPPPDVVNQDP
NNNLQEGTDPETEDPNHLPPDRDVLDGEQTSPSFMSTAWLVFKTFFASLL
PEGPPAIAN
Human HERPUD1 Protein sequence - var3
(public gi: 3005723)

(SEQ ID NO: 77)

GHLKAHLSRVYPERPRPEDQRLIYSGKLLLDHQCLRDLLPKEKRHVLHLV
CNVKSPSKMPEINAKVAESTEEPAGSNRGQYPEDSSSDGLRQREVLRNLS
SPGWENISRPEAAQQAFQGLGPGFSGYTPYGWLQLSWFQQIYARQYYMQY
LAATAASGAFVPPPSAQEIPVVSAPAPAPIHNQFPAENQPANQNAAPQVV
VNPGANQNLRMNAQGGPIVEEDDEINRDWLDWTYSAATFSVFLSILYFYS
SLSRFLMVMGATVVMYLHHVGWFPFRPRPVQNFPNDGPPPDVVNQDPNNN
LQEGTDPETEDPNHLPPDRDVLDGEQTSPSFMSTAWLVFKTFFASLLPEG
PPAIAN
Human HERPUD1 Protein sequence - var4
(public gi: 7661870)

(SEQ ID NO: 78)

MESETEPEPVTLLVKSPNQRHRDLELSGDRGWSVGHLKAHLSRVYPERPR
PEDQRLIYSGKLLLDHQCLRDLLPKQEKRHVLHLVCNVKSPSKMPEINAK
VAESTEEPAGSNRGQYPEDSSSDGLRQREVLRNLSSPGWENISRPEAAQQ
AFQGLGPGFSGYTPYGWLQLSWFQQIYARQYYMQYLAATAASGAFVPPPS
AQEIPVVSAPAPAPIHNQFPAENQPANQNAAPQVVVNPGANQNLRMNAQG
GPIVEEDDEINRDWLDWTYSAATFSVFLSILYFYSSLSRFLMVMGATVVM
YLHHVGWFPFRPRPVQNFPNDGPPPDVVNQDPNNNLQEGTDPETEDPNHL
PPDRDVLDGEQTSPSFMSTAWLVFKTFFASLLPEGPPAIAN
Rat HERPUD1 mRNA sequence
(public gi: 16758961)

(SEQ ID NO: 135)

AAGACACCAAGTGTCGTTGTGGGGTCGCAGACGGCTGCGTCGCCGCCCGT
TCGGCATCCCTGAGCGCAGTCGAGCCTCCACCGCCGCACACATGGAGCCC
GAGCCACAGCCCGAGCCGGTCACGCTGCTGGTGAAGAGCCCCAATCAGCG
CCACCGCGACTTCGAGCTGAGTGGCGACCGCGGTTGGAGTGTGAGTCGCC
TCAAGGCCCACCTGAGCCGAGTCTACCCCGAACGCCCGCGCCCAGAGGAC
CAGAGGTTAATTTATTCTGGGAAGCTGCTGTTGGATCACCAATGTCTCCA
AGACTTGCTTCCAAAGCAGGAAAAGCGACATGTTTTGCACCTCGTGTGCA
ATGTGACGAGTCCCTCAAAAAAGCCAGAAGCCAGCACAAAGGGTGCTGAG
TCCACAGAGCAGCCGGACAACACTAGTCAGGCACAGTATCCTGGGGATTC
CTCAAGCGATGGCTTACGGGAAAGGGAAGTCCTTCGGAACCTTCCTCCCT
CTGGATGGGAGAACGTCTCTAGGCCTGAAGCCGTCCAGCAGACTTTCCAA
GGCCTCGGGCCCGGCTTCTCTGGCTACACCACCTACCGGTGGCTGCAGCT
CTCCTGGTTCCAGCAGATCTATGCAAGACAGTACTACATGCAATACTTGG
CTGCCACTGCTGCTTCAGGAGCTTTTGGCCCTACACCAAGTGCACAAGAA
ATACCTGTGGTCTCTACACCGGCTCCCGCCCCTATACACAACCAGTTTCC
GGCAGAAAACCAGCCGGCCAATCAGAATGCACCCGCTCAAGCGGTTGTTA
ATCCCGGAGCCAATCAGAACTTGCGGATGAATGCACAAGGCGGCCCTCTG
GTGGAAGAAGATGATGAGATAAACCGAGACTCGTTGGATTGGACCTACTC
AGCAGCGACATTTTCCGTTTTCCTCAGCATTCTTTACTTCTACTCCTCCC
TGAGCAGATTCCTCATGGTCATGGGCGCCACCGTAGTCATGTACCTGCAC
CACGTCGGGTGGTTTCCATTCAGACAGAGGCCAGTTCAGAACTTCCCAGA
TGACGGTCCCCCTCAGGAAGCTGCCAACCAGGACCCCAACAATAACCTCC
AGGGAGGTTTGGACCCTGAAATGGAAGACCCCAACCGCCTCCCCGTAGGC
CGTGAAGTGCTGGACCCTGAGCATACCAGCCCCTCGTTCATGAGCACAGC
ATGGCTAGTCTTCAAGACTTTCTTTGCCTCTCTTCTTCCGGAAGGCCCAC
CAGCCCTAGCAAACTGATGGCCCCTGTGCTCTGTTGCTGGAGGCTTTCAC
AGCTTGGACTGGATCGTCCCCTGGCGTGGACTCGAGAGAGTCATTGAAAA
CCCACAGGATGACGATGTGCTTCTGTGCCAAGCAAAAGCACAAACTAAGA
CATGAAGCCGTGGTACAAACTGAACAGGGCCCCTCATGTCGTTATTCTGA
AGAGCTTTAATGTATACTGTATGTAGTCTCATAGGCACTGTAAACAGAAG
GCCCAGGGTCGCATGTTCTGCCTGAGCACCTCCCCAGACGTGTGTGCATG
TGTGCCGTACATGGAAGTCATAGACGTGTGTGCATGTGTGCTGTACATGG
AAGTCATAGATGCAGAAACGGTTCTGCTGGTTCGATTTGATTCCTGTTGG
AATGTTGCAATTACACTAAGTGTACTACTTTATATAATCAGTGACTTGCT
AGACATGTTAGCAGGAVTTTTCTAGGAGAGACTTATTGTATCATTGCTTT
TTAAAACGCAGTGCTTACTTACTGAGGGCGGCGACTTGGCACAGGTAAAG
CCTTTGCCGGGTTTTCTGTTCAATAAGTTTTGCTATGAACGACAAAAAAA
AAAAAAA
Rat HERPUD1 Protein sequence
(public gi: 16758962)

(SEQ ID NO: 136)

MEPEPQPEPVTLLVKSPNQRHRDLELSGDRGWSVSRLKAHLSRVYPERPR
PEDQRLIYSGKLLLDHQCLQDLLPKQEKRHVLMLVCNVRSPSKKPEASTK
GAESTEQPDNTSQAQYPGDSSSDGLREREVLRNLPPSGWENVSRPEAVQQ
TFQGLGPGFSGYTTYGWLQLSWFQQIYARQYYMQYLAATAASGAFGPTPS
AQEIPVVSTPAPAPIHNQFPAENQPANQNAAAQAVVNPGANQNLRMNAQG
GPLVEEDDEINRDWLDWTYSAATFSVFLSILYFYSSLSRFLMVMGATVVM
YLHHVGWFPFRQRPVQNFPDDGPPQEAANQDPNNNLQGGLDPEMEDPNRL
PVGREVLDPEHTSPSFMSTAWLVFKTFFASLLPEGPPALAN
Mouse HERPUD1 mRNA sequence
(public gi: 11612514)

(SEQ ID NO: 137)

AAAGACGCCAAGTGTCGTTGTGTGGTCTCAGACGGCTGCGTCGCCGCCCG
TTCGGCATCCCTGACCGCACTCGAGCCGCCAGCGACGCAGACATCGAGCC
CCAGCCACAGCCCGAGCCGGTCACGCTGCTGGTGAAGAGTCCCAATCAGC
GCCACCGCGACTTCGAGCTGAGTGGCGACCCCAGTTGGAGTGTGAGTCGC
CTCAAGGCCCACCTGAGCCGAGTCTACCCCGAGCGCCCGCGTCCAGAGGA
CCAGAGGTTAATTTATTCTGGGAAGCTGCTGTTGGATCACCAGTGTCTCC
AAGATTTGCTTCCAAAGCACGAAAAGCGACATGTTTTGCACCTTGTGTGC
AATGTGAAGAATCCCTCCAAAATGCCAGAAACCAGCACAAAGGGTGCTGA
ATCCACAGAGCAGCCGGACAACTCTAATCAGACACAGCATCCTGGGGACT
CCTCAAGTGATGGTTTACGGCAAAGACAAGTTCTTCGGAACCTTTCTCCC
TCCGGATGGGAGAACATCTCTAGGCCTGAGGCTGTCCAGCAGACTTTCCA
AGGCCTGGGGCCTGGCTTCTCTGGCTACACAACGTATGGGTGGCTGCAGC
TCTCCTGGTTCCAGCACATCTATGCAAGGCAGTACTACATGCAATACTTA
GCTGCCACTGCTGCATCAGGAACTTTTGTCCCGACACCAAGTGCACAAGA
GATACCTGTGGTCTCTACACCTGCTCCGGCTCCTATACACAACCAGTTTC
CGGCAGAAAACCAGCCGGCCAATCAGAATGCAGCTGCTCAAGCGGTTGTC
AATCCCGGAGCCAATCAGAACTTGCGGATGAATGCACAAGGTGGCCCCCT
GGTGGAGGAAGATGATGAGATAAACCGAGACTGGTTGGATTGGACCTATT
CCGCAGCGACGTTTTCTGTTTTCCTCAGCATCCTTTACTTCTACTCCTCG
CTGAGCAGATTTCTCATGGTCATGGGTGCCACTGTAGTCATGTACCTGCA
CCACGTCGGGTGGTTTCCGTTCAGACAGAGGCCAGTTCAGAACTTCCCGG
ATGATGGTGGTCCTCGAGATGCTGCCAACCAGGACCCCAACAATAACCTC
CAGGGAGGTATGGACCCAGAAATGGAAGACCCCAACCGCCTCCCCCCAGA
CCGCGAAGTGCTGGACCCTGAGCACACCAGCCCCTCGTTTATGAGCACAG
CATGGCTAGTCTTCAAGACTTTCTTTGCCTCTCTTCTTCCAGAAGGCCCA
CCAGCCCTAGCCAACTGATGGCCCTTGTGCTCTGTCGCTGGTGGCTTTGA
CAGCTCGGACTGGATCGTCTGGCTCCGGCTCCTTTTCCTCCCCTGGCGTG
GACTCGACAGAGTCATTGAAAACCCACAGGATGACATGTGCTTCTGTGCC
AAGCAAAAGCACAAACTAAGACATGAAGCCGTGGTACAAACTGAACAGGG
CCCCTCATGTCGTTATTCTGAAGAGCTTTAATGTATACTGTATGTAGTTT
CATAGGCACTGTAAGCAGAAGGCCCAGGGTCGCATGTTCTGCCTGAGCAC
CTCCCCAGATGTGTGTGCATGTGTGCTGTACATGGAAGTCATAGACGTGT
GTGCATGTGTGCTCTACATGGAAGTCATAGATGCAGAAACGGTTCTGCTG
GTTCGATTTGATTCCTGTTGGAATGTTCAAATTACACTAAGTGTACTACT
TTATATAATCAGTGAATTGCTAGACATGTTAGCACGACTTTTCTAGGAGA
GACTTATGTATAATTGCTTTTTAAAATGCAGTCCTTTCCTTTAAACCGAG
GGTGGCGACTTGGCAGAGGTAAAACCTTTGCCGAGTTTTCTGTTCAATAA
ACTTTTGCTATGAATGACTGT
Mouse HERPUD1 Protein sequence
(public gi: 11612515)

(SEQ ID NO: 138)

MEPEPQPEPVTLLVKSPNQRHRDLELSGDRSWSVSRLKAHLSRVYPERPR
PEDQRLIYSGKLLLDHQCLQDLLPKQEKRHVLHLVCNVKNPSKMPETSTK
GAESTEQPDNSNQTQHPGDSSSDGLRQREVLRNLSPSGWENISRPEAVQQ
TFQGLGPGPSGYTTYGWLQLSWFQQIYARQYYMQYLAATAASGTFVPTPS
AQEIPVVSTPAPAPIHNQFPAENQPANQNAAAQAVVNPGANQNLRMNAQG
GPLVEEDDEINRDWLDWTYSAATFSVFLSILYFYSSLSRFLMVMGATVVM
YLHHVGWFPFRQRPVQNFPDDGGPRDAANQDPNNNLQGGMDPEMEDPNRL
PPDREVLDPEHTSPSFMSTAWLVFKTFFASLLPEGPPALAN
Human GOCAP1 mRNA sequence - var1
(public gi: 10438060)

(SEQ ID NO: 47)

GATACGTGGCTGCCGTCTGTCCCCGCTGAGGAGGTGCAGCAGCCGGAGAT
GGCGGCGGTGCTGAACGCAGAGCGACTCGAGGTGTCCGTCGACGGCCTCA
CGCTCAGCCCGGACCCGGAGGAGCGGCCTGCGGCGGAGGGCGCCCCGCTG
CTGCCGCCACCGCTGCCACCCCCCTCGCCACCTCGATCCGGTCGCGGCCC
GGGCGCCTCAGGGGAGCAGCCCGAGCCCGGGGAGGCGGCGGCTGGGGGCG
CGGCGGAGGAGGCCCGGCGGCTGGAGCAGCGCTGGGGTTTCGGCCTCGAG
GAGTTGTACGGCCTGGCACTGCGCTTCTTCAAAGAAAAAGATGGCAAAGC
ATTTCATCCAACTTATGAAGAAAAATTGAAGCTTGTGGCACTGCATAACC
AAGTTCTTATGGGCCCATATAATCCAGACACTTGTCCTGAGGTTGGATTC
TTTGATGTGTTGGGGAATGACAGGAGGAGAGAATGGGCAGCCCTGGGAAA
CATGTCTAAAGAGGATGCCATGGTGGAGTTTGTCAAGCTCTTAAATAGGT
GTTGCCATCTCTTTTCAACATATGTTGCGTCCCACAAAATAGAGAAGGAA
GAGCAAGACAAAAAAAGGAAGGACGAAGAGGAGCGAAGGCGGCGTGAAGA
GGAAGAAAGAGAACGTCTGCAAAAGGAGGAAGAGAAACGTAGGAGAGAAG
AAGAGGAAAGGCTTCGACGGGAGGAAGAGGAAAGGAGACGGATAGAAGAA
GAAAGGCTTCGGTTGGAGCAGCAAAAGCAGCAGATAATGGCAGCTTTAAA
CTCCCAGACTGCCGTGCAGTTCCAGCAGTATGCAGCCCAACAGTATCCAG
GGAACTACGAACAGCAGCAAATTCTCATCCGCCAGTTGCAGGAGCAACAC
TATCAGCAGTACATGCAGCAGTTGTATCAAGTCCAGCTTGCACAGCAACA
GGCAGCATTACAGAAACAACAGGAAGTAGTAGTGGCTGGGTCTTCCTTGC
CTACATCATCAAAAGTGAATGCAACTGTACCAAGTAATATGATGTCAGTT
AATGGACAGGCCAAAACACACACTGACAGCTCCGAAAAAGAACTGGAACC
AGAAGCTGCAGAAGAAGCCCTGGAGAATGGACCAAAAGAATCTCTTCCAG
TAATAGCAGCTCCATCCATGTGGACACGACCTCAGATCAAAGACTTCAAA
GAGAAGATTCAGCAGGATGCAGATTCCGTGATTACAGTGGGCCGAGGAGA
AGTGGTCACTGTTCGAGTACCCACCCATGAAGAAGGATCATATCTCTTTT
CGGAATTTGCCACAGACAATTATGACATTGGGTTTGGGGTGTATTTTGAA
TGGACAGACTCTCCAAACACTGCTGTCAGCGTGCATGTCAGTGAGTCCAG
CGATGACGACGAGGAGGAAGAAGAAAACATCGGTTGTGAAGAGAAAGCCA
AAAAGAATGCCAACAAGCCTTTGCTGGATGAGATTGTGCCTGTGTACCGA
CGGGACTGTCATGACGAGGTGTATGCTGGCAGCCATCAATATCCAGGGAG
AGGAGTCTATCTCCTCAAGTTTGACAACTCCTACTCTTTGTGGCGGTCAA
AATCAGTCTACTACACAGTCTATTATACTAGATAAAAATGTTGTTACAAA
GTCTCGAGTCTAGGGTTGGGCAGAAGATGACATTTAATTTGGAAATTTCT
TTTTACTTTTGTGGAGCATTAGAGTCACAGTTTACCTTATTGATATTGGT
CTGATGGTTTGTGAACTCTTGCTGGGAATCAAAATTTCCTTGAGACTCTT
TAGCATTCATACTTTGGGGTTAAAGGAGATTCCTCAGACTCATCCAGCCC
TTGGGTGCTGACCAGCAGAGTCACTAGTGGATGCTGAAGTTACATGAGCT
ACATGTTAAATATTTAAAGTCTCCAAAATAAAACACCCCAACGTTGACCT
TACCCGGCTGATGGTTAGCCCCTTGCTGCCTCCTCCATGTGTCTTATGAG
AGCCCGTAGTTACAGTGTCCTCTAATTTGAAATCCATAAGTTAACAAGTC
TATATCAGGTGCAGCTGGCTTTGATTAAAGGCCATTTTTAAAACTTAAAA
ACTCAACACCTCACAGATTATAAAAAAAAAAAAAAAAAAA
Human GOCAP1 mRNA sequence - var2
(public gi: 15826851)

(SEQ ID NO: 48)

GGAAGTCGATACGTGGCTGCCTTCTGTCCCCGCTGAGGAGGTGCAGCAGC
CGGAGATGGCGGCGGTGCTGAACGCAGAGCGACTCGAGGTGTCCGTCGAC
GGCCTCACGCTCAGCCCGGACCCGGAGGAGCGGCCTGGGGCGGAGGGCGC
CCCGCTGCTGCCGCCACCGCTGCCACCGCCCTCGCCACCTGGATCCGGTC
GCGGCCCGGGCGCCTCAGGGGAGCAGCCCGAGCCCGGGGAGGCGGCGGCT
GGGGGCGCGGCGGAGGAGGCGCGGCGGCTGGAGCACCGCTGGGGTTTCGG
CCTGGAGGAGTTGTACGGCCTGGCACTGCGCTTCTTCAAAGAAAAAGATG
GCAAAGCATTTCATCCAACTTATGAAGAAAAATTGAAGCTTGTGGCACTG
CATAAGCAAGTTCTTATGGGCCCATATAATCCAGACACTTGTCCTGAGGT
TGGATTCTTTGATGTGTTGGGGAATGACAGGAGGAGAGAATGGGCAGCCC
TGGGAAACATGTCTAAAGAGGATGCCATGGTGGAGTTTGTCAAGCTCTTA
AATAGGTGTTGCCATCTCTTTTCAACATATGTTGCGTCCCACAAAATAGA
GAAGGAAGAGCAAGAAAAAAAAAGGAAGGAGGAAGAGGAGCGAAGGCGGC
GTGAAGAGGAAGAAAGAGAACGTCTGCAAAAGGAGGAAGAGAAACGTAGG
AGAGAAGAAGAGGAAAGGCTTCGACGGGAGGAAGAGGAAAGGAGACGGAT
AGAAGAAGAAAGGCTTCGGTTGGAGCAGCAAAAGCAGCAGATAATGGCAG
CTTTAAACTCCCAGACTGCCGTGCAGTTCCAGCAGTATGCAGCCCAACAG
TATCCAGGGAACTACGAACAGCAGCAAATTCTCATCCGCCAGTTGCAGGA
GCAACACTATCAGCAGTACATGCAGCAGTTGTATCAAGTCCAGCTTGCAC
AGCAACAGGCAGCATTACAGAAACAACAGGAAGTAGTAGTGGCTGGGTCT
TCCTTGCCTACATCATCAAAAGTGAATGCAACTGTACCAAGTAATATGAT
GTCAGTTAATGGACAGGCCAAAACACACACTGACAGCTCCGAAAAAGAAC
TCGAACCAGAAGCTGCAGAAGAAGCCCTGGAGAATGGACCAAAAGAATCT
CTTCCAGTAATAGCAGCTCCATCCATGTGGACACGACCTCAGATCAAAGA
CTTCAAAGAGAAGATTCAGCAGGATGCAGATTCCGTGATTACAGTGGGCC
GAGGAGAAGTGGTCACTGTTCGAGTACCCACCCATGAAGAAGGATCATAT
CTCTTTTCGGAATTTGCCACAGACAATTATGACATTGGGTTTGGGGTGTA
TTTTGAATGGACAGACTCTCCAAACACTGCTGTCAGCGTGCATGTCAGTG
AGTCCAGCGATGACGACGAGGAGGAAGAAGAAAACATCGGTTGTGAAGAG
AAAGCCAAAAAGAATGCCAACAAGCCTTTGCTGGATGAGATTGTGCCTGT
GTACCGACGGGACTGTCATGAGGACGTGTATGCTGGCAGCCATCAATATC
CAGGGAGACGAGTCTATCTCCTCAACTTTGACAACTCCTACTCTTTGTGG
CGGTCAAAATCAGTCTACTACAGAGTCTATTATACTAGATAAAAATGTTG
TTACAAAGTCTGGAGTCTAGGGTTGGGCAGAAGATGACATTTAATTTGGA
AATTTCTTTTTACTTTTGTGGAGCATTAGAGTCACAGTTTACCTTATTCA
TATTGGTCTGATGGTTTGTGAACTCTTGCTGGGAATCAAAATTTCCTTGA
GACTCTTTAGCATTCATACTTTGGGGTTAAACGAGATTCCTCAGACTCAT
CCAGCCCTTGGGTGCTGACCAGCAGAGTCACTAGTGGATGCTGAAGTTAC
ATGAGCTACATGTTAAATATTTAAAGTCTCCAAAATAAAACACCCCAACG
TTGACCTTACCCGGCTGATGGTTAGCCCCTTGCTGCCTGCTCCATGTGTC
TTATGAGAGCCCGTAGTTACAGTGTCCTCTAATTTGAAATCCATAAGTTA
ACAAGTCTATATCAGGTGCATCTGGCTTTGATTAAAGGCCATTTTTAAAA
CTTAAAAACTCAACACCTCACAGATTATAATAGAAAAAGAAATGGCCTCA
GTTTGATCTCGTTCAGAATGACCCAGATTGTTTCTGCTTTGGGTGCAGCT
GTTTAGTTCAGAGTTATATTACAGAGAATTATTTTCTGAGATAATCTTAA
ACTAGAATGTTCAAAACTAATTGATAATTGAAGTATCAAGATACGTAGAA
CACCTCAGAGATTTTTCTTCAGGAACTTCCACAAACTTTGAATCCTTGTA
TCTTTATTTGGTATTCATACTACTAGTAGCAAAATACAGGTTTTTTGTTT
TGTTTTGTTTTGGCTTCATAGAGTATCTCAAATTGAAACTTTTCTGCACA
AAGAATAAAATTAAGGATTTTATAAACTCAAATTGGCACCTACTGAATTA
AAATACATAAAATCATTTAAATATAATTCAGCATATGGGAAGTAACATTG
CACTAATATGGAAATCACTGCCAGAGACAGTCTATTTTCTTTTAATTTGT
TACTACTTAGTCACAAACCCCACATTATTCCAGTTTGGAATTACTTATTA
AGGAGAATTGGAAATACATATGCCCATGCTTAAATTTTATAGCTTTAATT
TGTGTTATTTCTTTATTGACGGGAAGAGGTACATCTTTTTTTCCTTACTG
AAAACCAAATATGGATTAATTGCCTCAAATTTGTATAAAGTGATTGGCTA
GTGATTCTTGTTTTCAGGAAGGGAGAGTGGTATAGATAGAAAATGACAAA
GATCGCAATATACACTTAATGTTGTTATTGTATGTTGTTACTGAAGTACT
TAGATTTTTAAAATTTCAAATCCTAAATCACTTCTTGTAGGAGGGTTTTC
ATTAACTGCAGTATATACAGTTCACTACATATGGGTTGTTTGAGTTTTTT
GTGTGCTGTATTTCTTTCTGTTTTTTTAATACCTGGTTTTGTACATATCT
AACTCTGTTCTCTTTTGGTTGTTCAGAAACTGGATTTTTTTTTTTTCTTA
AGCAGTGCTTAATTTGTGTTTTTTAATTTTGATTCAGAAGTAGTCCCAGC
TCATAGGTGTTCATAACTGTTACATCCAGAACATTTGTCAGGCTCTCTGT
CAGCTTTTCATGTACATATGGTATAGAAACCATGGAGTTAGGCACTTCCC
TGGAATTTTTTTTTTTATGAGAAAAATACTGTATTTAAAATGTAAAATAA
ACTTTTAAAAAGCAGGCACTAATATATATTTCTTCCAGCCTTTGATTACA
AATTTGTCCTTGCACATGTTAAGATGAATTATCTCCTAAAAATATCATTG
TTCTTGGGAGCACTGTATGTTACTTTACATAGCAGCGGTTCCTGTCATGT
GTTCATGTTCAGAAATATTTTTGGTTTTAAACTTTCTTATTGCCTTTGGC
TGTTGATTAGTACAGTACAAGTTGCGATTTCAAAAAGATCTTGAAAGTAA
TATATTTAATCAATTAAAATGTTTATCTGTAAAAAAAAAAAAAAAAAA
Human GOCAP1 mRNA sequence - var3
(public gi: 15799258)

(SEQ ID NO: 49)

GGAAGTCGATACGTGGCTGCCTTCTGTCCCCGCTGAGGAGGTGCAGCAGC
CGGAGATGGCGGCGGTGCTGAACGCAGAGCGACTCGAGGTGTCCGTCGAC
GGCCTCACGCTCAGCCCGGACCCGGAGGAGCGGCCTGGGGCGGAGGGCGC
CCCGCTGCTGCCGCCACCGCTGCCACCGCCCTCGCCACCTGGATCCGGTC
GCGGCCCGGGCGCCTCAGGGGAGCAGCCCGAGCCCGGGGACGCGGCGGCT
GGGGGCGCGGCGGAGGAGGCGCGGCGGCTGGAGCAGCGCTGGGGTTTCGG
CCTGGACGAGTTGTACGGCCTGGCACTGCGCTTCTTCAAAGAAAAAGATG
GCAAAGCATTTCATCCAACTTATGAAGAAAAATTGAAGCTTGTGGCACTG
CATAAGCAAGTTCTTATGGGCCCATATAATCCAGACACTTGTCCTGAGGT
TGGATTCTTTGATGTGTTGGGGAATGACAGGAGGAGAGAATGGGCAGCCC
TGGGAAACATGTCTAAAGAGGATGCCATGGTGGAGTTTGTCAAGCTCTTA
AATAGGTGTTGCCATCTCTTTTCAACATATGTTGCGTCCCACAAAATAGA
GAAGGAAGAGCAAGAAAAAAAAAGGAAGGAGGAAGAGGAGCGAAGGCGGC
GTGAAGAGGAAGAAAGAGAACGTCTGCAAAAGGAGGAAGAGAAACGTAGG
AGAGAAGAAGAGGAAAGGCTTCGACGGGAGGAAGAGGAAAGGAGACGGAT
ACAAGAAGAAAGGCTTCGGTTGGAGCAGCAAAAGCAGCAGATAATGGCAG
CTTTAAACTCCCAGACTGCCGTGCAGTTCCAGCAGTATCCAGCCCAACAG
TATCCAGGGAACTACGAACAGCAGCAAATTCTCATCCGCCAGTTGCAGGA
GCAACACTATCAGCAGTACATGCAGCAGTTGTATCAAGTCCAGCTTGCAC
ACCAACAGGCAGCATTACAGAAACAACAGGAAGTAGTAGTGGCTGGGTCT
TCCTTGCCTACATCATCAAAAGTGAATGCAACTGTACCAAGTAATATGAT
GTCAGTTAATGGACAGGCCAAAACACACACTGACAGCTCCGAAAAAGAAC
TGGAACCAGAAGCTGCAGAAGAAGCCCTGGAGAATGGACCAAAAGAATCT
CTTCCAGTAATAGCAGCTCCATCCATGTGGACACGACCTCAGATCAAAGA
CTTCAAAGAGAAGATTCAGCAGGATGCAGATTCCGTGATTACAGTGGGCC
GAGGAGAAGTGGTCACTGTTCGAGTACCCACCCATGAAGAAGGATCATAT
CTCTTTTGGGAATTTGCCACAGACAATTATGACATTGGGTTTGGGGTGTA
TTTTGAATGGACAGACTCTCCAAACACTGCTGTCAGCGTGCATGTCAGTG
AGTCCAGCGATGACGACGAGGAGGAAGAAGAAAACATCGGTTGTGAAGAG
AAAGCCAAAAAGAATCCCAACAACCCTTTGCTGGATGAGATTGTGCCTGT
GTACCGACGGGACTGTCATGAGGAGGTGTATGCTGGCAGCCATCAATATC
CAGGGAGAGGAGTCTATCTCCTCAAGTTTGACAACTCCTACTCTTTGTGG
CGGTCAAAATCAGTCTACTACAGAGTCTATTATACTAGATAAAAATGTTG
TTACAAAGTCTGGAGTCTAGGGTTGGGCAGAAGATGACATTTAATTTGGA
AATTTCTTTTTACTTTTGTGGAGCATTAGAGTCACAGTTTACCTTATTGA
TATTCGTCTGATGGTTTGTGAACTCTTGCTGGGAATCAAAATTTCCTTGA
GACTCTTTAGCATTCATACTTTGGGGTTAAAGGAGATTCCTCAGACTCAT
CCAGCCCTTGGCTGCTGACCAGCAGAGTCACTAGTGGATGCTGAAGTTAC
ATGACCTACATGTTAAATATTTAAAGTCTCCAAAATAAAACACCCCAACG
TTGACCTTACCCGGCTGATGGTTAGCCCCTTGCTGCCTGCTCCATGTGTC
TTATGAGAGCCCGTAGTTACAGTGTCCTCTAATTTGAAATCCATAAGTTA
ACAAGTCTATATCAGGTGCATCTGGCTTTGATTAAAGGCCATTTTTAAAA
CTTAAAAACTCAACACCTCACAGATTATAATAGAAAAAGAAATGGCCTCA
GTTTGATCTCGTTCAGAATGACCCAGATTGTTTCTGCTTTGGGTGCAGCT
GTTTAGTTCAGAGTTATATTACAGAGAATTATTTTCTGAGATAATCTTAA
ACTAGAATGTTCAAAACTAATTGATAATTGAAGTATCAAGATACGTAGAA
CACCTCAGAGATTTTTCTTCAGGAACTTCCACAAACTTTGAATCCTTGTA
TCTTTATTTGGTATTCATACTACTAGTAGCAAAATACAGGTTTTTTGTTT
TGTTTTGTTTTGGCTTCATAGAGTATCTCAAATTGAAACTTTTCTGCACA
AAGAATAAAATTAAGGATTTTATAAACTCAAATTGGCACCTACTGAATTA
AAATACATAAAATCATTTAAATATAATTCAGCATATGGGAAGTAACATTG
CACTAATATGGAAATCACTGCCAGAGACAGTCTATTTTCTTTTAATTTGT
TACTACTTAGTCACAAACCCCACATTATTCCAGTTTGGAATTACTTATTA
AGGAGAATTGGAAATACATATGCCCATGCTTAAATTTTATAGCTTTAATT
TGTGTTATTTCTTTATTGACGGGAAGAGGTACATCTTTTTTTCCTTACTG
AAAACCAAATATGGATTAATTGCCTCAAATTTGTATAAAGTGATTGGCTA
GTGATTCTTGTTTTCAGGAAGGGAGAGTGGTATAGATAGAAAATGACAAA
GATGGCAATATACACTTAATGTTGTTATTGTATGTTGTTACTGAAGTACT
TAGATTTTTAAAATTTCAAATCCTAAATCACTTCTTGTAGGAGGGTTTTC
ATTAACTGCAGTATATACAGTTCACTACATATGGGTTGTTTGAGTTTTTT
GTGTGCTGTATTTCTTTCTGTTTTTTTAATACCTGGTTTTGTACATATCT
AACTCTGTTCTCTTTTGGTTGTTCAGAAACTGGATTTTTTTTTTTTCTTA
AGCAGTGCTTAATTTGTGTTTTTTAATTTTGATTCAGAAGTAGTCCCAGC
TCATAGGTGTTCATAACTGTTACATCCAGAACATTTGTCAGGCTCTCTGT
CAGCTTTTCATGTACATATGGTATAGAAACCATGGAGTTAGGCACTTCCC
TGGAATTTTTTTTTTTATGAGAAAAATACTGTATTTAAAATGTAAAATAA
ACTTTTAAAAAGCAGGCACTAATATATATTTCTTCCAGCCTTTGATTACA
AATTTGTCCTTGCACATGTTAAGATGAATTATCTCCTAAAAATATCATTG
TTCTTGGGAGCAGTGTATGTTACTTTACATAGCAGCGGTTCCTGTCATGT
GTTCATGTTCAGAAATATTTTTGGTTTTAAACTTTCTTATTGCCTTTGGC
TGTTGATTAGTACAGTACAAGTTGCGATTTCAAAAAGATCTTGAAAGTAA
TATATTTAATCAATTAAAATGTTTATCTGTAAAAAAAAAAAAAAAAAA
Human GOCAP1 mRNA sequence - var4
(public gi: 21961496)

(SEQ ID NO: 50)

CGGACGCGTGGGTGCCATCTCTTTTCAACATATGTTGCGTCCCACAAAAT
AGAGAAGGAAGAGCAAGAAAAAAAAAGGAAGGAGGAAGAGGACCGAAGGC
CGCGTGAAGAGGAAGAAAGAGAACGTCTGCAAAAGGAGGAAGAGAAACGT
AGGAGAGAAGAAGAGGAAAGGCTTCGACGGGAGGAAGAGGAAAGGAGACG
GATAGAAGAAGAAAGGCTTCGGTTGGAGCAGCAAAAGCAGCAGATAATGG
CAGCTTTAAACTCCCAGACTGCCGTGCAGTTCCAGCAGTATGCAGCCCAA
CAGTATCCAGGGAACTACGAACAGCAGCAAATTCTCATCCGCCAGTTGCA
GGAGCAACACTATCAGCAGTACATGCACCAGTTGTATCAAGTCCAGCTTG
CACAGCAACAGGCAGCATTACAGAAACAACAGGAAGTAGTAGTGGCTCGG
TCTTCCTTGCCTACATCATCAAAAGTGAATGCAACTGTACCAAGTAATAT
GATGTCAGTTAATGGACAGGCCAAAACACACACTGACAGCTCCGAAAAAG
AACTGGAACCAGAAGCTGCAGAAGAAGCCCTGGAGAATGGACCAAAAGAA
TCTCTTCCAGTAATAGCAGCTCCATCCATGTGGACACGACCTCAGATCAA
AGACTTCAAAGAGAAGATTCAGCAGGATGCAGATTCCGTGATTACAGTGG
GCCGAGGAGAAGTGGTCACTGTTCGAGTACCCACCCATGAAGAAGGATCA
TATCTCTTTTGGGAATTTGCCACAGACAATTATGACATTGGGTTTGGGGT
GTATTTTGAATGGACAGACTCTCCAAACACTGCTGTCAGCGTGCATCTCA
GTGAGTCCAGCGATGACGACGAGGAGGAAGAAGAAAACATCGGTTGTGAA
GAGAAAGCCAAAAAGAATGCCAACAAGCCTTTGCTGGATGAGATTGTGCC
TGTGTACCGACGGGACTGTCATGAGGAGGTGTATGCTGGCAGCCATCAAT
ATCCAGGGAGAGGAGTCTATCTCCTCAAGTTTGACAACTCCTACTCTTTG
TGGCGGTCAAAATCAGTCTACTACAGAGTCTATTATACTAGATAAAAATG
TTGTTACAAAGTCTGGAGTCTAGGGTTGGGCAGAAGATGACATTTAATTT
GGAAATTTCTTTTTACTTTTGTGGAGCATTAGAGTCACAGTTTACCTTAT
TGATATTGGTCTGATGGTTTGTGAACTCTTGCTGGGAATCAAAATTTCCT
TGAGACTCTTTAGCATTCATACTTTGGGGTTAAAGGAGATTCCTCAGACT
CATCCAGCCCTTGGGTGCTGACCAGCAGAGTCACTAGTGGATGCTGAAGT
TACATGAGCTACATGTTAAATATTTAAAGTCTCCAAAATAAAACACCCCA
ACGTTGACCTTACCCGGCTGATGGTTAGCCCCTTGCTGCCTGCTCCATGT
GTCTTATGAGAGCCCGTAGTTACAGTGTCCTCTAATTTGAAATCCATAAG
TTAACAAGTCTATATCACGTGCAGCTGGCTTTGATTAAAGGCCATTTTTA
AAACTTAAAAACTCAACACCTCACAGATTATAATAGAAAAAGAAATGGCC
TCAGTTTGATCTCGTTCAGAATGACCCAGATTGTTTCTGCTTTGGGTGCA
GCTGTTTAGTTCAGAGTTATATTACAGAGAATTATTTTCTGAGATAATCT
TAAACTAGAATGTTCAAAACTAATTGATAATTGAAGTATCAAGATACGTA
GAACACCTCAGAGATTTTTCTTCAGGAACTTCCACAAACTTTGAATCCTT
GTATCTTTATTTGGTATTCATACTACTAGTAGCAAAATACAGGTTTTTTG
TTTTGTTTTGTTTTGTTTTGGCTTCATAGAGTATCTCAAATTGAAACTTT
TCTGCACAAAGAATAAAATTAAGGATTTTATAAACTCAAATTGGCACCTA
CTGAATTAAAATACATAAAATCATTTAAATATAATTCAGCATATGGGAAG
TAACATTGCACTAATATGGAAATCACTGCCAGAGACAGTCTATTTTCTTT
TAATTTGTTACTACTTAGTCACAAACCCCACATTATTCCAGTTTGGAATT
ACTTATTAAGGAGAATTGGAAATACATATGCCCATGCTTAAATTTTATAG
CTTTAATTTGTGTTATTTCTTTATTGACGGGAAGAGGTACATCTTTTTTT
CCTTACTCAAAACAAATATGGATTAATTGCCTCAAATTTGTATAAGTGAT
TGGCTAGTGATTCTTGTTTTCAGAAGGGAGAGTGGTATAGATAGAAAATG
ACAAAGATGGCAATATACACTTAATGTTGTTATTGTATGTTGTTACTGAA
GTACTTAGATTTTTAAAATTTCAAATCCTAAATCACTTCTTGTAGGAGGG
TTTTCATTAACTGCAGTATATACAGTTCACTACATATGGGTTGTTTGAGT
TTTTTGTGTGCTGTATTTCTTTCTGTTTTTTAATACCTGGTTTTGTACAT
ATCTAACTCTGTTCTCTTTTGGTTGTTCAGAAACTGGATTTTTTTTTTTC
TTAAGCAGTGCTTAATTTGTGTTTTTTAATTTTGATTCAGAAGTAGTCCC
AGCTCATAGGTGTTCATACTGTTACATCCAGAACATTTGTCAGGCTCTCT
GTCAGCTTTCATGTACATATGGTATAGAAACCATGGAGTTAGGCACTTCC
TGGATTTTTTTTTTATGAGAAAAATACTGTATTTAAAATGTAAAATAAAC
TTTTAAAAAGCAGGCACTAATATATATTTCTTCCAGCCTTTGATTACAAA
TTTGTCCTTGCACATGTTAAGATGAATTATCTCCTAAAAATATCATTGTT
CTTGGGAGCAGTGTATGTTACTTTACATAGCAGCGGTTCCTGTCATGTGT
TCATGTCAGAGTATTTTTGGTTTTAAACTTTCTTATTGCCTTTGGCTGTT
GATTAGTACAGTACAAGTGCGATTTCAAAAAGATCTTGAAAGTAATATAT
TTAATCAATTAAAATGTTTATCTGTAAAAAAAAAAAAAAAAAAAAAAAA
Human GOCAP1 mRNA sequence - var5
(public gi: 24496472)

(SEQ ID NO: 51)

CCGCTGAGGAGGTGCAGCAGCCGGAGATGGCGGCGGTGCTGAACGCAGAG
CGACTCGAGGTGTCCGTCGACGGCCTCACGCTCAGCCCGGACCCGGAGGA
GCGGCCTGGGGCGGAGGGCGCCCCGCTGCTGCCGCCACCGCTGCCACCGC
CCTCGCCACCTGGATCCGGTCGCGGCCCGGGCGCCTCAGGGGAGCAGCCC
GAGCCCCGGGAGGCGGCCGCTGGGGGCGCGGCGGAGGAGGCGCGGCGGCT
GGAGCAGCGCTGGGGTTTCGGCCTGGAGGACTTGTACGGCCTGGCACTGC
GCCTCTTCAAAGAAAAAGATGGCAAAGCATTTCATCCAACTTATGAAGAA
AAATTGAAGCTTGTGGCACTGCATAAGCAAGTTCTTATGGGCCCATATAA
TCCAGACACTTGTCCTGAGGTTGGATTCTTTGATGTGTTGGGGAATGACA
GGAGGAGAGAATGGGCAGCCCTGGGAAACATGTCTAAAGAGGATGCCATG
GTGGAGTTTGTCAAGCTCTTAAATAGGTGTTGCCATCTCTTTTCAACATA
TGTTGCGTCCCACAAAATAGACAAGGAAGAGCAAGACAAAAAAAGGAAGG
AGGAAGAGGAGCGAAGGCGGCGTGAAGAGGAAGAAAGAGAGCGTCTGCAA
AAGGAGGAAGAGAAACGTAGGAGAGAAGAAGAGGAAAGGCTTCGACGGGA
GGAAGAGGAAAGGATACGGATAGAAGAAGAAAGGCTTCGGTTGGAGCAGC
AAAAGCAGCAGATAATGGCAGCTTTAAACTCCCAGACTGCCGTGCAGTTC
CACCACTATGCAGCCCAACGGTATCCAGGGAACTACGAACAGCAGCAAAT
TCTCATCCGCCAGTTGCAGGAGCAACACTATCAGCAGTACATGCAGCAGT
TGTATCAAGTCCAGCTTGCACAGCAACAGGCAGCATTACAGAAACAACAG
GAAGTAGTAGTCGCTGGGTCTTCCTTGCCTACATCATCAAAAGTGAATGC
AACTGTACCAAGTAATATGATGCCAGTTAATGGACAGGCCAAAACACACA
CTGACACCTCCGAAAAAGAACTGGAACCAGAAGCTGCAGAAGAAGCCCTG
GAGAATGGACCAAAAGAATCTCTTCCAGTAATAGCAGCTCCATCCATGTG
GACACGACCTCAGATCAAAGACTTTCAAAGAGAAGATTCAGCAGGATGCA
GATTCCGTGATTACAGTGGCCGAGGAGAAGTCGTCACTGTTCGAGTACCC
ACCCATGAAGAAGGATCATATCTCTTTTGGGAATTTGCCACAGACAATTG
TGACATTGGGTTTGGGGTGTATTTTGAATGGACAGACTCTCCAAACACTG
CTGTCAGCGTGCATGTCAGTGAGTCCAGCGATGACGACGAGGAGGAAGAA
GAAAACATCGGTTGTGAAGAGAAAGCCAAAAAGAATGCCAACAAGCCTTT
GCTGGATGAGATTGTGCCTGTGTACCGACGGGACTGTCATGAGGAGGTGT
ATGCTGGCAGCCATCAATATCCAGGGAGAGGAGTCTATCTCCTCAAGTTT
GACAACTCCTACTCTTTGTGGCGGTCAAAATCAGTCTACTACAGAGTCTA
TTATACTAGATAAAAATGTTGTTACAAAGTCTGGAGTCTAGGGTTGGGCA
GAAGATGACATTTAATTTGGAAATTTCTTTTTACTTTTGTGGAGCATTAG
AGTCACAGTTTACCTTATTGATATTGGTCTGATGGTTTGTGAACTCTTGC
TGGGAATCAAAATTTCCTTGAGACTCTTTAGCATTCATACTTTGGGGTTA
AAGGAGATTCCTCAGACTCATCCAGCCCTTGGGTGCTGACCAGCAGAGTC
ACTAGGGGATCCTGAAGTTACATGAGCTACATGTTAAATATTTAAAGTCT
CCAAAATAAAACACCCCAACGTTGACCTTACCCGGCTGATGGTTAGCCCC
TTGCTGCCTGCTCCATGTGTCTTATGAGAGCCCGTAGTTACAGTGTCCTC
TAATTTGAAATCCATAAGTTAACAAGTCTATATCAGGTGCAGCTGGCTTT
GATTAAAGGCCATTTTTAAAACTTAAAAACTCAACACCTCACAGATTATA
ATAGAAAAAGAAATGGCCTCAGCTTGATCTCGTTCAGAATGACCCAGATT
GTTTCTGCCTTGGGTGCAGCTGTTTAGTTCAGAGTTATATTACAGAGAAT
TATTTTCTGAGATAATCTTAAACTAGAATGTTCAAAACTAATTGATAATT
GAAGTATCAAGATACGTAGAACACCTCAGAGATTTTTCTTCAGGAACTTC
CACAAACTTTGAATCCTTGTATCTTTATTTGGTATTCATACTACTAGTAG
CAAAATACACGTTTTTTGTTTTGTTTTGTTTTGTTTTGGCTTCATAGAGT
ATCTCAAATTGAAACTTTTCTGCACAAAGAATAAAATTAAGGATTTTATA
AACTCGAATTGGCACCTACTGAATTAAAATACATAAAATCATTTAAATAT
AATTCAGCATATGGGAAGTAACATTGCACTAATATCGAAATCACTGCCAG
AGACACTCTATTTTCTTTTAATTTGTTACTACTTAGTCACAACCCCACAT
TATTCCAGTTTCGAATTACTTATTAAGGAGAATTGGAAATACATGTGCCC
ATGCTTAAATTTTATAGCTTTAATTTGTGTTATTTCTTTATTGACGGGAA
GAGGTACATCTTTTTTTCCTTACTGAAAACAAATATGGATTAATTGCCTC
AAATTTGTATAAAGTGATTGGCTAGTGATTCTTGTTTTCAGAGGGGAGAG
TGGTATAGATAGAAAATGACGAAGATGGCAATATACACTTAATGTTGTTA
TTGTATGTTGTTACTCAAGACTTAGATTTTTAAAATTTCAAATCCTAAAT
CACTTCTTGTAGGGGGGTTTTCATTAACTGCAGTATATACAGTTCACTAC
ATATGGGTTGTTTGAGTTTTTTGTGTGCTGTATTTCTTTCTGTTTTTTAA
TACCTGGTTTTGTACATATCTAACTCTGTTCTCTTTTGGTTGTTCAGAAA
CTGGATTTTTTTCTTCTTAAGCAGTGCTTAATTTGTGTTTTTTAATTTTG
ATTCAGAAGTAGTCCCAGCTCATAGGCGTTCATACTGTTACATCCAGAAC
ATTTGTCAGGCTCTCTGTCAGCTTTCATGTACATATGGTATAGAAACCAT
GGAGTTAGGCACTTCCTGGATTTTTTTTATGAGAAAAATNCTGTATTTAA
AATGTAAAATAAACTTTTAAAAAGCAGGCACTAATATATATTTCTTCCAG
CCTTTGATTACAAATTTGTCCTTGCACATGTTAAGATGAATTATCTCCTA
AAAATATCATTGTTCTTGGGAGCAGTCTATCTTACTTTACATAGCAGCGG
TTCCTGTCATGTGTTCATGTCACGAATATTTTTGGTTTTAAACTTTCTTA
TTGCCTTTCGCTGTTGATTAGTACAGTACAAGTGCGATTTCAAAAAGATC
TTGAAGTAATATATTTAATCAATTAAAATGTTTATCTGGAAAAAAAAAAA
AAAAAAAAAAAAAAAAAAAAAA
Human GOCAP1 mRNA sequence - var6
(public gi: 28374435)

(SEQ ID NO: 52)

TCCGTCCCCGCTGAGGAGGTGCAGCAGCGGGAGATGGCGGCGGTGCTGAA
CGCAGAGCGACTCGAGGTGTCCGTCGACGGCCTCACGCTCAGCCCGGACC
CGGAGGAGCGGCCTGGGGCGGAGGGCGCCCCGCTGCTGCCGCCACCGCTG
CCACCGCCCTCGCCACCTGGATCCGGTCGCGGCCCGGGCGCCTCACGGGA
GCAGCCCGAGCCCCGGGAGGCGGCGGCTGGGGGCGCGGCGGAGGAGGCGC
GGCGGCTGGAGCAGCGCTGGGGTTTCGGCCTGGAGGAGTTGTACGGCCTG
GCACTGCGCTTCTTCAAAGAAAAAGATCGCAAAGCATTTCATCCAACTTA
TGAAGAAAAATTGAAGCTTGTGGCACTGCATAAGCAAGTTCTTATCGGCC
CATATAATCCAGACACTTGTCCTGAGGTTGGATTCTTTGATGTGTTGGGG
AATGACAGGAGGAGAGAATGGGCAGCCCTGGGAAACATGTCTAAAGAGGA
TGCCATGGTGGAGTTTGTCAAGCTCTTAAATAGGTGTTGCCATCTCTTTT
CAACATATGTTGCGTCCCACAAAATAGAGAAGGAAGAGCAAGAAAAAAAA
AGGAAGGAGGAAGAGGAGCGAAGGCGGCGTGAAGAGGAAGAAAGAGAACG
TCTGCAAAAGGAGGAAGAGAAACGTAGGAGAGAAGAAGAGGAAAGGCTTC
GACGGGAGGAAGAGGAAAGGAGACGGATAGAAGAAGAAAGGCTTCGGTTG
GAGCAGCAAAAGCAGCAGATAATGGCAGCTTTAAACTCCCAGACTGCCGT
GCAGTTCCAGCAGTATGCAGCCCAACAGTATCCAGGGAACTACGAACAGC
AGCAAATTCTCATCCGCCAGTTGCAGGAGCAACACTATCAGCAGTACATG
CAGCAGTTGTATCAAGTCCAGCTTGCACAGCAACAGGCAGCATTACAGAA
ACAACAGGAAGTAGTACTGGCTGGGTCTTCCTTGCCTACATCATCAAAAG
TGAATGCAACTGTACCAAGTAATATGATGTCACTTAATGGACAGGCCAAA
ACACACACTGACAGCTCCGAAAAAGAACTGGAACCGGAAGCTGCAGAAGA
AGCCCTGGAGAATGGACCAAAAGAATCTCTTCCAGTAATAGCAGCTCCAT
CCATGTGGACACGACCTCAGATCAAAGACTTCAAAGAGAAGATTCAGCAG
GATGCAGATTCCGTGATTACAGTGGGCCGAGGAGAAGTGGTCACTGTTCG
AGTACCCACCCATGAAGAAGGATCATATCTCTTTTGGGAATTTGCCACAG
ACAATTATGACATTGGGTTTGGGGTGTATTTTGAATGGACAGACTCTCCA
AACACTGCTGTCAGCGTGCATGTCAGTGAGTCCAGCGATGACGACGAGGA
GGAAGAAGAAAACATCGGTTGTGAAGAGAAAGCCAAAAAGAATGCCAACA
AGCCTTTGCTGGATGAGATTGTGCCTGTGTACCGACGGGACTGTCATGAG
GAGGTGTATGCTGGCAGCCATCAATATCCAGGGAGAGGACTCTATCTCCT
CAAGTTTGACAACTCCTACTCTTTGTGGCGGTCAAAATCAGTCTACTACA
GAGTCTATTATACTAGATAAAAATGTTGTTACAAAGTCTGGAGTCTAGGG
TTGGGCAGAAGATGACATTTAATTTGGAAATTTCTTTTTACTTTTGTGGA
GCATTAGAGTCACAGTTTACCTTATTGATATTGGTCTGATGGTTTGTGAA
CTCTTGCTGGGAATCAAAATTTCCTTGAGACTCTTTAGCATTCATACTTT
GGGGTTAAAGGAGATTCCTCAGACTCATCCAGCCCTTGGGTGCTGACCAG
CAGAGTCACTAGTGGATGCTGAAGTTACATGAGCTACATGTTAAATATTT
AAAGTCTCCAAAATAAAACACCCCAACGTTGACCTTACCCGGCTGATGGT
TAGCCCCTTGCTGCCTGCTCCATGTGTCTTATGAGAGCCCGTAGTTACAG
TGTCCTCTAATTTCAAATCCATAAGTTAACAAGTCTATATCAGGTCCAGC
TGGCTTTGATTAAAGGCCATTTTTAAAACTTAAAAACTCAACACCTCACA
GATTATAATAGAAAAAGAAATGGCCTCAGTTTGATCTCGTTCAGAATGAC
CCAGATTGTTTCTGCTTTGGGTGCAGCTGTTTAGTTCAGAGTTATATTAC
AGAGAATTATTTTCTGAGATAATCTTAAACTAGAATGTTCAAAACTAATT
GATAATTGAAGTATCAAGATACGTAGAACACCTCAGAGATTTTTCTTCAG
GAACTTCCACAAACTTTGAATCCTTGTATCTTTATTTGGTATTCATACTA
CTAGTAGCAAAATACAGGTTTTTTGTTTTGTTTTGTTTTGGCTTCATAGA
GTATCTCAAATTGAAACTTTTCTCCACAAAGAATAAAATTAAGGATTTTA
TAAACTCAAATTGGCACCTACTGAATTAAAATACATAAAATCATTTAAAT
ATAATTCAGCATATGGGAAGTAACATTGCACTAATATGGAAATCACTGCC
AGAGACAGTCTATTTTCTTTTAATTTGTTACTACTTAGTCACAAACCCCA
CATTATTCCAGTTTGGAATTACTTATTAAGGAGAATTGGAAATACATATC
CCCATGCTTAAATTTTATAGCTTTAATTTGTGTTATTTCTTTATTGACGG
GAAGAGGTACATCTTTTTTTCCTTACTGAAAACAAATATGGATTAATTGC
CTCAAATTTGTATAAGTGATTGGCTACTGATTCTTGTTTTCAGAAGGGAG
AGTGGTATAGATAGAAAATGACAAAGATGGCAATATACACTTAATGTTGT
TATTGTATGTTGTTACTGAAGTACTTAGATTTTTAAAATTTCAAATCCTA
AATCACTTCTTGTAGGAGGGTTTTCATTAACTGCAGTATATACAGTTCAC
TACATATCGGTTGTTTGAGTTTTTTGTGTGCTGTATTTCTTTCTGTTTTT
TAATACCTGGTTTTGTACATATCTAACTCTGTTCTCTTTTGGTTGTTCAG
AAACTGGATTTTTTTTTCTTAAGCAGTGCTTAATTTGTGTTTTTTAATTT
TGATTCAGAAGTAGTCCCACCTCATAGGTGTTCATACTGTTACATCCAGA
ACATTTGTCAGGCTCTCTGTCAGCTTTCATGTACATATGGTATAGAAACC
ATGGAGTTAGGCACTTCCTGGATTTTTTTTTTTATGAGAAAAATACTGTA
TTTAAAATGTAAAATAAACTTTTAAAAAGCAGGCACTAATATATATTTCT
TCCAGCCTTTGATTACAAATTTGTCCTTGCACATGTTAAGATGAATTATC
TCCTAAAAATATCATTGTTCTTGGGAGCAGTGTATGTTACTTTACATAGC
AGCGGTTCCTGTCATGTGTTCATGTCAGAATATTTTTGGTTTTAAACTTT
CTTATTGCCTTTGGCTGTTGATTAGTACAGTACAAGTGCGATTTCAAAAA
GATCTTGAAAGTAATATATTTAATCAATTAAAATGTTTATCTGTCAAAAA
AAAAAAAAAA
Human GOCAP1 mRNA sequence - var7
(public gi: 25058702)

(SEQ ID NO: 53)

CGCTGAGGAGGTGCAGCAGCCGGAGATGGCGGCGGTGCTGAACGCAGAGC
GACTCGAGGTGTCCGTCGACGGCCTCACGCTCAGCCCGGACCCGGAGGAG
CGGCCTGGGGCGGAGGGCGCCCCGCTGCTGCCGCCACCGCTGCCACCGCC
CTCGCCACCTGGATCCGGTCGCGGCCCGGGCGCCTCAGGGGAGCAGCCCG
AGCCCGGGGAGGCGGCGGCTGGGGGCGCGGCGGAGGAGGCGCGGCGGCTG
GAGCAGCGCTGGGGTTTCGGCCTGGAGGAGTTGTACGGCCTGGCACTGCG
CTTCTTCAAAGAAAAAGATGGCAAAGCATTTCATCCAACTTATGAAGAAA
AATTGAAGCTTGTGGCACTGCATAAGCAAGTTCTTATGGGCCCATATAAT
CCAGACACTTGTCCTGAGGTTGGATTCTTTGATGTGTTGGGGAATGACAG
GAGGAGAGAATGGGCAGCCCTGGGAAACATGTCTAAAGAGGATGCCATGG
TGGAGTTTGTCAAGCTCTTAAATAGGTGTTGCCATCTCTTTTCAACATAT
GTTGCGTCCCACAAAATAGAGAAGGAAGAGCAAGAAAAAAAAAAGGAAGG
ACGAAGACGAGCGAAGGCGGCGTGAAGAGGAAGAAAGAGAACGTCTGCAA
AAGGAGGAAGAGAAACGTAGGAGAGAAGAAGAGGAAAGGCTTCGACGCGA
GGAAGACGAAAGGAGACGGATAGAAGAAGAAAGGCTTCGGTTGGAGCAGC
AAAAGCAGCAGATAATGGCAGCTTTAAACTCCCAGACTGCCGTGCAGTTC
CAGCAGTATGCAGCCCAACAGTATCCAGGGAACTACGAACAGCAGCAAAT
TCTCATCCGCCAGTTGCAGGAGCAACACTATCAGCAGTACATGCAGCAGT
TGTATCAAGTCCAGCTTGCACACCAACAGGCAGCATTACAGAAACAACAG
GAAGTAGTACTGGCTGGGTCTTCCTTGCCTACATCATCAAAAGTGAATGC
AACTGTACCAAGTAATATGATGTCAGTTAATGGACAGGCCAAAACACACA
CTGACAGCTCCGAAAAAGAACTGGAACCAGAAGCTGCAGAAGAAGCCCTG
GAGAATGGACCAAAAGAATCTCTTCCAGTAATAGCAGCTCCATCCATGTG
GACACGACCTCAGATCAAAGACTTCAAAGAGAAGATTCAGCAGGATGCAG
ATTCCGTGATTACAGTGGGCCGAGGAGAAGTGGTCACTGTTCGAGTACCC
ACCCATGAAGAAGGATCATATCTCTTTTGGGAATTTGCCACAGACAATTA
TGACATTGGGTTTCGGGTGTATTTTGAATGGACAGACTCTCCAAACACTG
CTGTCAGCGTGCATGTCAGTGAGTCCAGCGATGACGACGAGGAGGAAGAA
GAAAACATCGGTTGTGAAGAGAAAGCCAAAAAGAATCCCAACAAGCCTTT
GCTGGATGAGATTGTGCCTGTGTACCGACGGGACTGTCATGAGGAGGTGT
ATCCTGGCAGCCATCAATATCCAGGGAGAGGAGTCTATCTCCTCAAGTTT
GACAACTCCTACTCTTTGTCGCGGTCAAAATCAGTCTACTACAGAGTCTA
TTATACTAGATAAAAATGTTGTTACAAAGTCTGGAGTCTAGGGTTGGGCA
GAAGATGACATTTAATTTGGAAATTTCTTTTTACTTTTGTGGAGCATTAG
AGTCACAGTTTACCTTATTGATATTGGTCTGATGGTTTGTGAACTCTTGC
TGGGAATCAAAATTTCCTTGAGACTCTTTAGCATTCATACTTTGGGCTTA
AAGGAGATTCCTCAGACTCATCCAGCCCTTGGGTGCTGACCAGCAGAGTC
ACTAGTGGATGCTGAAGTTACATGAGCTACATGTTAAATATTTAAAGTCT
CCAAAATAAAACACCCCAACGTTGACCTTAAAAAAAAAAAAAAAAAAAAA
AAAAAAAAAAAAAAAAAAAAAAAAAAAAAAAAAAAAAAAAAAAA
Human GOCAP1 mRNA sequence - var8
(public gi: 2738926)

(SEQ ID NO: 54)

GAATTCCGTTGCTGTCGGAGCCCGTAGTTACAGTGTCCTCTAATTTGAAA
TCCATAAGTTACCAAGTCTATATCAGGTACAGCTGGCTTTCATTAAAGGC
CATTTTTAAAACTTCAAAAACTCAACACCTCACAGATTATAATAGAAAAA
GAAATGGCCTCAGTTTGATCTCGTTCAGAATGACCCACATTGTTTTCTGC
TTTCCGTGCAGCTGTTTAGTTCAGAGTTATATTACAGAGAATTATTTTCT
GAGAAATCTTAAAACTAGAATGTTCAAAACTAATTCGATAATTGAAGTAT
CAAGATACGTAGAACACCTCACAGATTTTTCTTCAGGAACTTCCACAAAC
TTTAGAATCCTTGTATCTTTATTTGGTATTCATACTACTAGTCGCAAAAT
ACAGGTTTTTTGTTTTGTTTTGTTTTGTTTTGGCTTCATAGAGTATCTCA
AATTGAAACTTTTCTGCCCAAAGAATAAAATTAAGGATTTTATAAAACTC
AAATTGGCACCTACTGAATTAAAATACATAAAATGCATTAAATATAATTC
AGCATATGGCAGTAACATTGCACTAATATGGAAATCACTGCCAGAGACAG
TCTATTTTCTTTTAATTTGTTACTACTTAGTCACAACCCCACATTATTCC
AGTTTGGAATTACTTATTAAGGAGAATTGGAAATACATATGCCCATGCTT
AAATTTTATAGCTTTAATTTGTGTTATTTCTTTATTGACGGGAAGAGGTA
CATCTTTTTTTCCTTACTCAAAACAAATATGGATTAATTGCCTCAAATTT
GTATAAGTGATTGGCTAGTGATTCTTGTTTTCAGAGGGAGAGTGGTATAG
ATAGAAAATGACAAAGATGGCAATATACACTTAATGTTGTTATTGTATGT
TGTTACTGAAGTACTTAGATTTTTAAAATTTCAAATCCTAAATCACTTCT
TGTAGGAGGGTTTTCATTAACTCCAGATATACAGTTCACTACATATGGGT
TGTTTGAGTTTTTTGTGTGCTGTATTTCTTTCTGTTTTTTAATACCTGGT
TTTGTACATATCTAACTCTGTTCTCTTTTGGTTGTTCAGAAACTGGATTT
TTTTTTTCTTAAGCAGTGCTTAATTTGTGTTTTTTAATTTTGATTCAGAA
GTAGTCCCAGCTCATAGGTGTTCATACTCTTACATCCAGAACATTTGTCA
GGCTCTCTGTCAGCTTTCATGTACATATGGTATAGAAACCATGGAGTTAG
GCACTTCCTGGATTTTTTTTTTATGAGAAAAATACTGTATTTAAAATGTA
AAATAAACTTTTAAAAAGC
Human GOCAP1 Protein sequence - var1
(public gi: 24496473)

(SEQ ID NO: 79)

MAAVLNAERLEVSVDGLTLSPDPEERPGAEGAPLLPPPLPPPSPPGSGRG
PGASGEQPEPGEAAAGGAAEEARRLEQRWGFGLEELYGLALRLFKEKDGK
AFHPTYEEKLKLVALHKQVLMGPYNPDTCPEVGFFDVLGNDRRREWAALG
NMSKEDAMVEFVKLLNRCCHLFSTYVASHKIEKEEQDKKRKEEEERRRRE
EEERERLQKEEEKRRREEEERLRREEEERIRIEEERLRLEQQKQQIMAAL
NSQTAVQFQQYAAQRYPGNYEQOQILIRQLQEQHYQQYMQQLYQVQLAQQ
QAALQKQQEVVVAGSSLPTSSKVNATVPSNMMPVNGQAKTHTDSSEKELE
PEAAEEALENGPKESLPVIAAPSMWTRPQIKDFQREDSAGCRFRDYSGRG
EVVTVRVPTHEEGSYLFWEFATDNCDIGFGVYFEWTDSPNTAVSVHVSES
SDDDEEEEENIGCEEKAKKNANKPLLDEIVPVYRRDCHEEVYAGSHQYPG
RGVYLLKFDNSYSLWRSKSVYYRVYYTR
Human GOCAP1 Protein sequence - var2
(public gi: 21961497)

(SEQ ID NO: 80)

RTRGCHLFSTYVASHKIEKEEQEKKRKEEEERRRREEEERERLQKEEEKR
RREEEERLRREEEERRRIEEERLRLEQQKQQIMAALNSQTAVQFQQYAAQ
QYPGNYEQQQILIRQLQEQHYQQYMQQLYQVQLAQQQAALQKQQEVVVAG
SSLPTSSKVNATVPSNMMSVNGQAKTHTDSSEKELEPEAAEEALENGPKE
SLPVIAAPSMWTRPQIKDFKEKIQQDADSVITVGRGEVVTVRVPTHEEGS
YLFWEFATDNYDIGFGVYFEWTDSPNTAVSVHVSESSDDDEEEEENIGCE
EKAKKNANKPLLDEIVPVYRRDCHEEVYAGSHQYPGRGVYLLKFDNSYSL
WRSKSVYYRVYYTR
Human GOCAP1 Protein sequence - var3
(public gi: 15799259)

(SEQ ID NO: 81)

MAAVLNAERLEVSVDGLTLSPDPEERPGAEGAPLLPPPLPPPSPPGSGRG
PGASGEQPEPGEAAAGGAAEEARRLEQRWGFGLEELYGLALRFFKEKDGK
AFHPTYEEKLKLVALHKQVLMGPYNPDTCPEVGFFDVLGNDRRREWAALG
NMSKEDAMVEFVKLLNRCCHLFSTYVASHKIEKEEQEKKRKEEEERRRRE
EEERERLQKEEEKRRREEEERLEREEEERRRIEEEELRLEQQKQQIMAAL
NSQTAVQFQQYAAQQYPGNYEQQQILIRQLQEQHYQQYMQQLYQVQLAQQ
QAALQKQQEVVVAGSSLPTSSKVNATVPSNMMSVNGQAKTHTDSSEKELE
PEAAEEALENGPKESLPVIAAPSMWTRPQIKDFKEKIQQDADSVITVGRG
EVVTVRVPTHEEGSYLFWEFATDNYDIGFGVYFEWTDSPNTAVSVHVSES
SDDDEEEEENIGCEEKAKKNANKPLLDEIVPVYRRDCMEEVYAGSHQYPG
RGVYLLKFDNSYSLWRSKSVYYRVYYTR
Human GOCAP1 Protein sequence - var4
(public gi: 10438061)

(SEQ ID NO: 82)

MAAVLNAERLEVSVDGLTLSPDPEERPGAEGAPLLPPPLPPPSPPGSGRG
PGASGEQPEPGEAAAGGAAEEARRLEQRWGFGLEELYGLALRFFKEKDGK
AFHPTYEEKLKLVALHKQVLMGPYNPDTCPEVGFFDVLGNDRRREWAALG
NMSKEDAMVEFVKLLNRCCHLFSTYVASHKIEKEEQEKKRKEEEERRRRE
EEERERLQKEEEKRRREEEERLEREEEERRRIEEEELRLEQQKQQIMAAL
NSQTAVQFQQYAAQQYPGNYEQQQILIRQLQEQHYQQYMQQLYQVQLAQQ
QAALQKQQEVVVAGSSLPTSSKVNATVPSNMMSVNGQAKTHTDSSEKELE
PEAAEEALENGPKESLPVIAAPSMWTRPQIKDFKEKIQQDADSVITVGRG
EVVTVRVPTHEEGSYLFWEFATDNYDIGFGVYFEWTDSPNTAVSVHVSES
SDDDEEEEENIGCEEKAKKNANKPLLDEIVPVYRRDCMEEVYAGSHQYPG
RGVYLLKFDNSYSLWRSKSVYYRVYYTR
Rat GOCAP1 mRNA sequence
(public gi: 27679381)

(SEQ ID NO: 139)

CAAGACCCACACTGAGAATCCCGAGAAAGTCCTTGAGCCAGAAGCTGCGG
AAGAGGCCTTGGAAAATGGACCCAAAGACTCTCTTCCAGTGATTGCAGCT
CCTTCCATGTGGACAAGACCACAGATCAAAGACTTTAAAGAGAAGATTCG
GCAGGATGCAGATTCTGTGATTACAGTCCGGCGAGGAGAAGTAGTCACCG
TTCGAGTCCCCACTCATGAGGAAGGATCATACCTCTTTTGGGAATTTGCC
ACAGACAGTTATGACATTGGGTTTGGGGTTTATTTTGAATGGACAGACTC
TCCAAACGCTGCTGTCAGTGTGCATGTCAGTGAGTCCAGTGACGACGAAG
AGGAGGAGGAAGAAGGTAGGCCTTGGTTCACATCTAGTCCTGCTAACACT
CATAGACAGGAAGTGCTCTGTTCAGGATTGGGATAGTGGGTGAAAAGACT
CTGAGAAAGGCCTGCCTGTCACAAAGGCCCTAAACATGAATTTCTCTAAA
ATGCCAAGAAAGTCTGTTTGGTTCTGAGTGTTGTTTTACTTCAGTCTAGT
GTGTTGGGAAGCTAAGTATCTGAAGACCCTTAGAGGAGCGGAGTTGTTTG
TATATTTTGTTTTTGAGACAGGAACTTACTTTCTTGTCCAGGCTGGTCTT
GAACTGGTGAACTCAAAACAGCCCTCTTCCTCACCCTCCCTAGTAGCTGG
GACTACTGGTGTATGCCACCATGCCCCAGTTTGTAAAGGTTTGAAAGAGC
AAGTAAGAATTCTTGCCAACAAAAGAATTTATATTTCCGATAACAACGTT
TTTGTTTGAAAGACAGGATCCCTCTGTTAATTTGAGGACAGCCTGGTCT
Rat GOCAP1 Protein sequence
(public gi: 27679382)

(SEQ ID NO: 140)

MWTRPQIKDFKEKIRQDADSVITVRRGEVVTVRVPTHEEGSYLFWEFATD
SYDIGFGVYFEWTDSPNAAVSVHVSESSDDEEEEEEGRPWFTSSPANTHR
QEVLCSGLG
Mouse GOCAP1 mRNA sequence
(public gi: 22831366)

(SEQ ID NO: 141)

GAGCAGGAAGTCGATACGTGGCCGCCGCCTGTCCCCGCCGAGGAGGCGCT
GCGACCGGAGATGGCGGCGCAGCTGAACGTAGAGCAGCTCGAGGTGTCGC
TCGACGGTCTCACGCTGAGCCCGGACTCGGAGGAGAGGCCCGGCGCGGAG
GGGGCCCCGCCGCAGACGCCGCCGTCCTCGGCCCCGGGGAATGGACTGGG
CTCCGGCGCCTCGGGGCAGCAGCGGGAGCCCGGGGAGGCGGCGGCTGAGG
GCGCGGCCGACGAGGCGCGACGGATGGAGCAGCACTGGGGCTTTGGCCTC
GAGGAGCTTTACGGCCTGGCGCTGCGCTTCTACAAAATAAAAGATGGCAA
AGCCTTTCATCCAACTTATGAAGAAAAACTGAAGTTCGTCGCACTGCATA
AGCAAGTTCTTTTGGGCCCATATAACCCAGACACGTCCCCTGAGGTTGGA
TTCTTTGATGTGTTGGGGAATGATAGGAGGAGAGAATGGGCAGCTCTGGG
AAACATCTCCAAGGAGGATGCCATGGTAGAGTTTGTGAACCTTCTAAATA
AGTGTTGTCCTCTCCTCTCGGCATATGTTGCGTCCCACAGAATAGAGAAG
GAAGAAGAAGAGAAAAGAAGAAACGCGGAGGAGGAGCGAACGCAGCGTGA
AGAGGAAGAACGAGAGCGGCTGCAAAAGGAAGAAGAGAAGCGGAAGCGAG
AGGAGGAAGACCGGCTGAGACGGGAGGAGGAAGAGAGGCGGCGGATAGAG
GAAGAGAGGCTTCGGCTGGAACACCAAAAGCAGCAGATAATGGCAGCTTT
AAACTCGCAGACTGCCGTGCAATTCCAGCAGTATGCAGCCCAGCAGTATC
CAGGGAACTACGAACAACAGCAGATTCTCATCCGCCAGCTGCAGGACCAG
CACTATCAGCAGTATATGCAGCAGTTATATCAAGTCCAGCTTGCACAACA
ACAGGCAGCATTACAGAAACAGCAAGAAGTAGTGATGGCTGGGGCATCAT
TGCCTGCATCATCAAAGGTGAACACAGCTGGAGCAAGTGATACACTGTCA
GTTAATGGACAGGCCAAAACCCACACTGAAAATTCCGAAAAAGTCCTTGA
GCCAGAAGCTGCAGAAGAAGCCTTGGAAAATGGACCAAAAGACTCTCTTC
CAGTGATTGCAGCTCCATCCATGTGGACAAGACCACAAATCAAAGACTTT
AAAGAGAAGATTCGGCAGGATGCAGATTCTGTGATTACAGTACGTCGAGG
AGAAGTCGTCACCGTCCGAGTCCCGACTCATGAGGAAGGATCATACCTAT
TTTGGGAATTTGCCACAGACAGTTATGACATTGGGTTTGGGGTTTATTTT
GAATGGACAGACTCTCCAAATGCTGCTGTCAGTGTGCATGTCAGTGAGTC
CAGTGACGAGGAGGAGGAGGAGGAAGAAAATGTCACTTGTGAAGAAAAAG
CAAAAAAGAACGCCAACAAGCCTCTGCTGGATGAGATTGTACCTGTGTAC
CGGCGGGACTGTCACGAGGAAGTATATGCAGGCAGCCACCAGTATCCAGC
GAGGGGAGTCTATCTCCTCAAGTTTGATAATTCCTACTCTCTGTGGAGGT
CCAAGTCCGTCTACTACAGAGTCTATTATACTAGATAGAGCTGCTGTTCC
AGGGTCGGGAGTCTAGGGTTGAGCACAACATGACGTTTAATTTCCTTTGA
AAAAAAAAAAAAAAAAAAAAAAAA
Mouse GOCAP1 Protein sequence
(public gi: 22831367)

(SEQ ID NO: 142)

MAAQLNVEQLEVSLDGLTLSPDSEERPGAEGAPPQTPPSSAPGNGLGSGA
SGQQREPGEAAAEGAAEEARRMEQHWGFGLEELYGLALRFYKIKDGKAFH
PTYEEKLKFVALHKQVLLGPYNPDTSPEVGFFDVLGNDRRREWAALGNMS
KEDAMVEFVKLLNKCCPLLSAYVASHRIEKEEEEKRRKAEEERRQREEEE
RERLQKEEEKRKREEEDRLRREEEERRRIEEERLRLEQQKQQIMAALNSQ
TAVQFQQYAAQQYPGNYEQQQILIRQLQEQHYQQYMQQLYQVQLAQQQAA
LQKQQEVVMAGASLPASSKVNTAGASDTLSVNGQAKTHTENSEKVLEPEA
AEEALENGPKDSLPVIAAPSMWTRPQIKDFKEKIRQDADSVITVRRGEVV
TVRVPTHEEGSYLFWEFATDSYDIGFGVYFEWTDSPNAAVSVHVSESSDE
EEEEEENVTCEEKAKKNANKPLLDEIVPVYRRDCHEEVYAGSHQYPGRGV
YLLKFDNSYSLWRSKSVYYRVYYTR
Drosophila GOCAP1 mRNA sequence
(public gi: 24643365)

(SEQ ID NO: 143)

ACTTTAGGTGCGCAGCTGGCTCGTCACACTGGCAGCAGACGTCACAATAA
ATTCTATTATTTTGTTTTGTTTTGTGCGAAAAGTTTGCAGGCCAGCAATC
GTGGACTAGCCCACAAACAACGGCAGCATGGCGAGCGGGACAAGCGGCAC
AAGCAGCAGTGAAACGGCCCAGTCGACGGACATGGCACCATCATCGGCCG
AGAAGTGGGGCTTCCCTTTGATTGAGCTCTACCGCCTGGCATTCACGTTC
TACAACCGGAACTCCGGCAAGGCCATCCACTTGTCGTACGAGGACAACCT
CAAGCTGATCGCCTTCAAGCAGCAGGCGGCGCTGGGCCCCTTCAACACGA
GTCGCGCCCCGGCGCTGGGCGTCCTGGACGTGATCGGACGCGACCGGCAG
CAGCACTGGCAGCTGCTCGGTGAGATAACGCGCGAGCACGCGATGGAGGG
GTTCGTCGACCTGCTGGACACAATGTGCAGCGCCTTTCGGCCGTACATTG
AGGCCGTGCGGCAGGACCGCGACGAGACGCTGCGCAAGGAGCTGCGCCTC
ATGGAGGAGAAGAACGAGGCACGCGAGCGGCAGGAGAACGCCCAGCAGGA
GCTGCTCGACGACGGTTACAAGGACGAGCTGCAGCGCCGCCAGCTGCAGG
ACGCACTCAACAAGCAGACTTACCAGCAGTTTAAGCTGTACGCTGAGAAG
CAGTTCCCCGGAAACCCCGAACAGCAGGCCGTGCTTATCCACCAGCTGCA
GCGGGAGCACTATCACCAGTACATGCAACAGCTTCATCTGCAGAACCAGA
ACCAGAATCAGAATCAGAACACGGAGGACAAGGGACACCAGGAAGCAGAA
CACGTCCCTGGCAATAACAACAACAGCTCAACCGACTTGCCCAACGCCAT
GGAGGGACTGAAGCTCGCTCAGGTCGAGACACAGCAGCACCAGCAGCTGG
CCGAGCAGCAAGACGGGCATGTAGAGCAGACGTTGGAGGGCGTTGGGCAG
GAACAGCACGGCGAGGAAGAGTACGACGACTACGTTATGATCTGCCCGGC
CAAGATCTGGACTCGTCCCGACATCGACCAGTTTAAGACTGAGGTGTCCG
CCGGCGATGGGGACGGCGTCATCACCATTCGACACGGCGACACGGTGACG
GTCCGTGTGCCCACCAACATGAACGGCAAGTGCATCTTCTGGGAGTTCGC
CACGGACAGCTACGACATCGGCTTCGGCATCTACTTCGAGTGGGCCAAGC
CCGTGACCAACGAGGTGACCGTGCATGTGAGCGACTCCGACGAGGACGAG
GATTGCGTGGACGAGGACTACCTATCCACCACCGAGGACCTGGAGTCCGG
CTCGCTGTCGCAGGAGCGCGGCGCAGTTAACAACCCGACTGCTGCCCCCA
AGGCACCCATTTCCATTATCGTACCGATCTATCGACGGGAGTGCTACAAC
GAGGTCTACGTGGGCTCCCACTCCTATCCGGGCGAGGGCGTCTATTTGCT
GAAGTTCGACAACAGCTACAGCATCTGGCGCAATAAGACGCTGTACTACC
GCGTCTACTACGAGCGTTAAGAGGCCAAAATGTATTTACTTACCACTAAC
TTAGATGAATCCTATCTCCGTACGAACGGCTAATGTGCAAGTCCGGAATA
ATCTGTATGTGCGTGTGCGTGAGGTTTTGGTTTTACGTGTGCGTTGCTGC
AATTCCGGGAAACAAGCGGCGGGAAAAGACCATGCCGATTTCAGACTAAA
CCAATATTATTTATTATTTAGTTGTAACCGACGAAGACAAGTCCAAAATT
CAATTTGAAACTAATAAACAATATAAAGACAACTGTAAATAA
Drosophila GOCAP1 Protein sequence
(public gi: 19920370)

(SEQ ID NO: 144)

MASGTSGTSSSETAQSTDMAPSSAEKWGFPLIELYRLAFTFYKRNSGKAI
HLSYEDNLKLIAFKQQAALGPFNTSRAPALGVLDVIGRDRQQHWQLLGEI
TREQAMEGFVDLLDTMCSAFRPYIEAVRQDRDETLRKELRLMEEKKEARE
RQENAQQELLEEGYKEELQRRQLQDALNKQTYQQFKLYAEKQFPGNPEQQ
AVLIHQLQREHYHQYMQQLHLQNQNQNQNQNTEDKGHQEAEHVPGNNNNS
STDLPNAMEGLKLGEVETQQHQQLAEQQDGHVEQTLEGVGQEQHGEEEYD
DYVMICPAKIWTRPDIEQFKTEVSAGDGDGVITIGHGDTVTVRVPTNMNG
KCIFWEFATDSYDIGFGIYFEWAKPVTNEVTVHVSDSDEDEDCVDEDYLS
TTEDLESGSLSQERGAVNNPTAAPKAPISIIVPIYRRECYNEVYVGSHSY
PGEGVYLLKFDNSYSIWRNKTLYYRVYYER
C. elegans GOCAP1 mRNA sequence
(public gi: 17543337)

(SEQ ID NO: 145)

ATGGCCGACGAGAACCACCTCCGTCTAAGCGAGGTTCAACTGATTCAGTC
GGAATTCGGACATTCTCTCGAGGAATGCTACAAGCTTGCCGTTCAATATT
ATAAAAAAGAACACGTCGGGAAACAGGAGCCTGTTGGATATGAAGATCGC
ATTAAATTGCTCTCGTTGAGCAAACAAGTCCAACATGGAGAGATTTCCGA
TGAATTTGACAACGCTGGATGGCTAGATATCACTGGAAACGATGTGAACA
AAGCTTGGCGTGAGCTGGGCTCGTTGTCCCGCGACGAAGCGATGGCCTCG
TTCGTCTTCCTCGTCGATCGTGTGTGTCCGCCGTTCAAGGGATTCATTGC
CGATAAGAAGGCTATAAAAGACGCGGAACTGAAAGAATTCGCGCCACAGG
TCACTGAACAAAGCGCCCAGCCACCGTCTCTTCAACAAGTTGATCAGAGA
CTTTTCGAGGATCAGCGGAAGCAAATCCAAGAAGCACTCAACGCACAGAC
CTTCCATCAATTCTCAGCCTACGCTCAGGAGCAATTCCCAGGACAACCGG
AACAACAGACGACACTTATTCGACAGCTCCAAGAGCAACACTATCAACAA
TACATGTCTCAGGTCTATGCTCAACAATCGACTACGCCGAACGGTGCAGA
AATGAATCCAGAGGAGTCGCATCAACATCAGATTCGTCGTGATGATGATT
CGGATGTTTCGGATGATGAAGCTGGGGAGGATTTGCCTTCCAACCCTGCA
ATTTCGCCCGCCTCACTCTGGAATCGGCAGGATATTAATGAGTTCAAGGC
GAATATCAAGAAAGACGGACACGAGGGAATTATTAAAGTTGGACACGGCG
AGACGGTGACGGTCCGGGTCCCAACTCACGAAAATGGTTCATGTCTATTC
TGGGAATTCGCGACGGATCACTACGATATTGGTTTCGGTGTCTACTTTGA
ATGGACCGTAGCCGACAGTAATCAAGTTTCGGTGCACGTTAGTGAATCCG
ATGATGAGGAGGATTATGATGAAGCACTTGAAGCGGAGCAGGCGGAAGCT
GGAGGTGGTGGTGGAGGAGCCGCTGGACAGGGTGGACCAGGAGATGTGGA
AGCTGGAGCCATGCAGACAAGACGTGTTGATCCGAACAAGCCGCGGCAGG
ATGAAATAATTCCGGTGTATCGTCGAGATTGTCACGAAGAAGTGTATGCG
GGAAGTCATCGTTATCCGGGTCGCGGAATTTATCTGCTCAAATTCCACAA
TTCGTATTCTTTGTGGCGTTCGAAGACGCTCTACTATCGTGTCTACTACA
GTAAATAA
C. elegans GOCAP1 Protein sequence
(public gi: 17543338)

(SEQ ID NO: 146)

MADENNVRLSEVQLIQSEFGHSLEECYKLAVQYYKKEHVGKQEPVGYEDR
IKLLSLSKQVQHGEISDEFDNAGWLDITGNDVNKAWRELGSLSRDEAMAS
FVFLVDRVCPPFKGFIADKKAIKDAELKEFAPQVTEQSAQPPSLQQVDQR
LFEDQRKQIQEALNAQTFHQFSAYAQEQFPGQPEQQTTLIRQLQEQHYQQ
YMSQVYAQQSTTPNGAEMNPEESHQHQIRRDDDSDVSDDEAGEDLPSNPA
ISPASLWNRQDINEFKANIKKDGHEGIIKVGHGETVTVRVPTHENGSCLF
WEFATDHYDIGFGVYFEWTVADSNQVSVHVSESDDEEDYDEALEAEQAEA
GGGGGGAAGQGGPGDVEAGAMQTRRVDPNKPRQDEIIPVYRRDCHEEVYA
GSHRYPGRGIYLLKFDNSYSLWRSKTLYYRVYYSK
Human CBL-B mRNA sequence - var1
(public gi: 4757919)

(SEQ ID NO: 55)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGCGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TCCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CACGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAACGAGACAATGTATGAAGAACAGTCACAGGACAGACCAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACCGCATGGCAGGAGTCGGATGGTCAGGGCTGCCCTT
TCTGTCGTTGTGAAATAAAAGGAACTGAGCCCATAATCCTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACCCCATGATTTGCCTTTAGAAGGAGCTAACGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCATATATTTAAAGGGTACGTATAGAATATAATTTCCTTTGTGATGT
ACATCTTAATGGTCAGAATTTAAAGGCAAAATTTCATGCCATTGTACTGA
AAATACATTAAGCTTTTGTGTTATCCTCTAGGAGATGTTTTTGATTCAGC
CTCTGATCCCGTGCCATTACCACCTGCCAGGCCTCCAACTCGGGACAATC
CAAAGCATGGTTCTTCACTCAACAGGACGCCCTCTGATTATGATCTTCTC
ATCCCTCCATTAGGTTGAAACCTTTAAAAAAGTTTTGAACAACCCACCCC
TCCTTCTTTTAATTTCAGAATTTTCAGAATTCAGAGTTCAGTATAACACA
GACTCACTGGGTTGTGAATTTGCCTGAAATTTGAATGGGTTCTCCAGGTG
CCGGTGACTCCCAAGTTCACGAGACCATTACTCCATGTAGATGATTAAGG
TAGTAGTGTAGTAGTTGGGCATCAGTCAGGTTTTAAGCAAGTTGTTTTGT
CCATACTAAATGTAGTCTAAAAACACATGAGAGCTTTGTGCTCTAGTAGT
TTTGAAGTGATGACTTGAAGTGTTGAGATTTTCTTTAAGTATAATAATTC
TTAATAAATATGAACTTGCTTTTCTTGCAGCATGAGCACCAGTTCCACTT
ACGCTAATTAAATTATGCAAAATTAAATAGTTGTATGTAGAGAACTGATA
ATAAATTCTGTTTTATTCTAATCATTACAACTGTAACACATTCAAAAAAA
AAAA
Human CBL-B mRNA sequence - var2
(public gi: 23273908)

(SEQ ID NO: 56)

AGCGGAGTGCTGCTGCGGCGTCCCGCGGCCTCCCCGAGTCGGGCGGGAGG
GGACAGCGGGTGTGGATTTGTCTTGACGGTAATTGTTGCGTTTCCACGTC
TCGGAGGCCTGCGCGCTGGGTTGCTCCTTCTTCGGGAGCCAGCTGTTCTC
AGCGATCCCACTCCCAGCCGGGGCTCCCCACACACACTGGGCTGCGTGCG
TGTGGAGTGGGACCCGCGCACACGCGTGTCTCTGGACAGCTACGGCGCCG
AAAGAACTAAAATTCCAGATGGCAAACTCAATGAATGGCAGAAACCCTGG
TGGTCGAGGAGGAAATCCCCGAAAAGGTCGAATTTTGGGTATTATTGATG
CTATTCAGGATGCAGTTGGACCCCCTAAGCAAGCTGCCGCAGATCGCAGG
ACCGTGGAGAACACTTGGAAGCTCATGGACAAAGTGGTAAGACTGTGCCA
AAATCCCAAACTTCAGTTGAAAAATAGCCCACCATATATACTTGATATTT
TGCCTGATACATATCAGCATTTACGACTTATATTGAGTAAATATGATGAC
AACCAGAAACTTGCCCAACTCAGTGAGAATGAGTACTTTAAAATCTACAT
TGATAGCCTTATGAAAAAGTCAAAACGGGCAATAAGACTCTTTAAAGAAG
GCAAGGAGAGAATGTATGAAGAACAGTCACAGGACAGACGAAATCTCACA
AAACTGTCCCTTATCTTCAGTCACATGCTGGCAGAAATCAAAGCAATCTT
TCCCAATGGTCAATTCCACGGAGATAACTTTCGTATCACAAAACCAGATG
CTGCTGAATTCTGGAGAAAGTTTTTTGGAGACAAAACTATCGTACCATGG
AAAGTATTCAGACAGTGCCTTCATGAGGTCCACCAGATTAGCTCTGGCCT
GGAAGCAATGGCTCTAAAATCAACAATTGATTTAACTTGCAATGATTACA
TTTCAGTTTTTGAATTTGATATTTTTACCAGGCTGTTTCAGCCTTGGGGC
TCTATTTTGCGGAATTGGAATTTCTTAGCTGTGACACATCCAGGTTACAT
GGCATTTCTCACATATGATGAAGTTAAAGCACGACTACAGAAATATAGCA
CCAAACCCGGAAGCTATATTTTCCGGTTAAGTTGCACTCGATTGGGACAG
TGGGCCATTGGCTATGTGACTGGGGATGGGAATATCTTACAGACCATACC
TCATAACAAGCCCTTATTTCAAGCCCTGATTCATGGCACCAGGGAAGGAT
TTTATCTTTATCCTGATGGGAGGAGTTATAATCCTGATTTAACTGGATTA
TGTGAACCTACACCTCATGACCATATAAAAGTTACACACGAACAATATGA
ATTATATTGTGAAATGGGCTCCACTTTTCAGCTCTGTAAGATTTGTGCAG
AGAATGACAAAGATGTCAAGATTGAGCCTTGTGGGCATTTGATGTGCACC
TCTTGCCTTACGGCATGGCAGGAGTCGGATGGTCAGGGCTGCCCTTTCTG
TCGTTGTGAAATAAAACGAACTGAGCCCATAATCGTGGATCCCTTTGATC
CAAGAGATGAAGGCTCCAGGTGTTGCAGCATCATTGACCCCTTTGGCATG
CCCATGCTCGACTTGGACGACGATGATGATCGTGAGGAGTCCTTGATGAT
GAATCGGTTGGCAAACGTCCGAAAGTGCACTGACAGGCAGAACTCACCAG
TCACATCACCAGGATCCTCTCCCCTTGCCCAGAGAAGAAAGCCACAGCCT
GACCCACTCCAGATCCCACATCTAAGCCTGCCACCCGTGCCTCCTCGCCT
GGATCTAATTCAGAAAGGCATAGTTAGATCTCCCTGTGGCAGCCCAACGG
GTTCACCAAAGTCTTCTCCTTGCATGGTGAGAAAACAAGATAAACCACTC
CCAGCACCACCTCCTCCCTTAAGAGATCCTCCTCCACCGCCACCTGAAAG
ACCTCCACCAATCCCACCAGACAATAGACTGAGTAGACACATCCATCATG
TGGAAAGCGTGCCTTCCAAAGACCCGCCAATGCCTCTTGAAGCATGGTCC
CCTCGGGATGTGTTTGGGACTAATCAGCTTGTGGGATGTCGACTCCTAGG
GGAGGGCTCTCCAAAACCTGGAATCACACCGAGTTCAAATGTCAATGGAA
GGCACAGTAGAGTGGGCTCTGACCCAGTGCTTATGCGGAAACACAGACCC
CATGATTTGCCTTTAGAAGGAGCTAAGGTCTTTTCCAATGGTCACCTTGG
AAGTGAAGAATATGATGTTCCTCCCCGGCTTTCTCCTCCTCCTCCAGTTA
CCACCCTCCTCCCTAGCATAAAGTGTACTGGTCCGTTAGCAAATTCTCTT
TCAGAGAAAACAAGAGACCCAGTAGAGGAAGATGATGATGAATACAAGAT
TCCTTCATCCCACCCTGTTTCCCTGAATTCACAACCATCTCATTGTCATA
ATGTAAAACCTCCTGTTCGGTCTTGTGATAATGGTCACTGTATGCTGAAT
GGAACACATGGTCCATCTTCAGAGAAGAAATCAAACATCCCTGACTTAAC
CATATATTTAAAGGGAGATGTTTTTGATTCACCCTCTGATCCCGTGCCAT
TACCACCTGCCAGGCCTCCAACTCGGGACAATCCAAAGCATGGTTCTTCA
CTCAACAGGACGCCCTCTGATTATGATCTTCTCATCCCTCCATTAGGTGA
AGATGCTTTTGATGCCCTCCCTCCATCTCTCCCACCTCCCCCACCTCCTG
CAAGGCATAGTCTCATTGAACATTCAAAACCTCCTGGCTCCAGTACCCGG
CCATCCTCAGGACAGGATCTTTTTCTTCTTCCTTCAGATCCCTTTGTTGA
TCTAGCAAGTGGCCAAGTTCCTTTGCCTCCCGCTAGAAGGTTACCAGGTG
AAAATGTCAAAACTAACAGAACATCACAGGACTATGATCAGCTTCCTTCA
TGTTCAGATGGTTCACAGGCACCAGCCAGACCCCCTAAACCACGACCGCG
CAGGACTGCACCAGAAATTCACCACAGAAAACCCCATGGGCCTGAGGCGG
CATTGGAAAATGTCGATGCAAAAATTGCAAAACTCATGGGAGAGGGTTAT
GCCTTTGAAGAGGTGAAGAGAGCCTTAGAGATAGCCCAGAATAATGTCGA
AGTTGCCCGGAGCATCCTCCGAGAATTTGCCTTCCCTCCTCCAGTATCCC
CACGTCTAAATCTATAGCAGCCAGAACTGTAGACACCAAAATGGAAAGCA
ATCGATGTATTCCAAGAGTGTGGAAATAAAGAGAACTGAGATGGAATTCA
AGAGAGAAGTGTCTCCTCCTCGTGTAGCAGCTTGAGAAGAGGCTTGGGAG
TGCAGCTTCTCAAAGGAGACCGATGCTTGCTCAGGATGTCGACAGCTGTG
GCTTCCTTGTTTTTGCTAGCCATATTTTTAAATCAGGGTTGAACTGACAA
AAATAATTTAAAGACGTTTACTTCCCTTGAACTTTGAACCTGTGAAATGC
TTTACCTTGTTTACAATTTGGCAAAGTTGCAGTTTGTTCTTGTTTTTAGT
TTAGTTTTGTTTTGGTGTTTTGATACCTGTACTGTGTTCTTCACAGACCC
TTTGTAGCGTGGTCAGGTCTGCTGTAACATTTCCCACCAACTCTCTTGCT
GTCCACATCAACAGCTAAATCATTTATTCATATGGATCTCTACCATCCCC
ATGCCTTGCCCAGGTCCAGTTCCATTTCTCTCATTCACAAGATGCTTTGA
AGGTTCTGATTTTCAACTGATCAAACTAATGCAAAAAAAAAAGTATGTAT
TCTTCACTACTGAGTTTCTTCTTTGGAAACCATCACTATTGAGAGATGGG
AAAAACCTGAATGTATAAAGCATTTATTTGTCAATAAACTGCCTTTTGTA
AGGGGTTTTCACAAAAAAAAAAAAAAAA
Human CBL-B mRNA sequence - var3
(public gi: 862406)

(SEQ ID NO: 57)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATCCTGCTGAATTCTGGAGAAAGTTTTTTGGAGACAAAACTATCGTACC
ATGGAAAGTATTCAGACAGTGCCTTCATGACGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTCTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAAGCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACCGCATGGCAGGAGTCGGATGGTCAGGGCTGCCCTT
TCTGTCGTTGTGAAATAAAAGGAACTGAGCCCATAATCGTGCACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAACCGTGCCTTCCAGAGACCCGCCAATGCCTCTTGAAGCATG
GTGCCCTCGGGATGTGTTTGGGACTAATCAGCTTGTCGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTCATAATGGTCACTGTATGCT
GAATGGAACACATGGTCCATCTTCAGAGAAGAAATCAAACATCCCTGACT
TAAGCATATATTTAAAGGGAGATGTTTTTGATTCAGCCTCTGATCCCGTG
CCATTACCACCTGCCAGGCCTCCAACTCCGGACAATCCAAAGCATGGTTC
TTCACTCAACAGGACGCCCTCTGATTATGATCTTCTCATCCCTCCATTAG
GTGAAGATGCTTTTGATGCCCTCCCTCCATCTCTCCCACCTCCCCCACCT
CCTGCAAGGCATAGTCTCATTGAACATTCAAAACCTCCTGGCTCCAGTAG
CCGGCCATCCTCAGGACAGGATCTTTTTCTTCTTCCTTCAGATCCCTTTG
TTGATCTAGCAAGTGGCCAAGTTCCTTTGCCTCCTGCTAGAAGGTTACCA
GGTGAAAATGTCAAAACTAACAGAACATCACAGGACTATGATCAGCTTCC
TTCATGTTCAGATGGTTCACAGGCACCAGCCAGACCCCCTAAACCACGAC
CGCGCAGGACTGCACCAGAAATTCACCACAGAAAACCCCATGGGCCTGAG
GCGGCATTGGAAAATGTCGATGCAAAAATTGCAAAACTCATGGGAGAGGG
TTATGCCTTTGAAGAGGTGAAGAGAGCCTTAGAGATAGCCCAGAATAATG
TCGAAGTTGCCCGGAGCATCCTCCGAGAATTTGCCTTCCCTCCTCCAGTA
TCCCCACGTCTAAATCTATAGCAGCCAGAAGTGTAGACACCAAAATGGAA
AGCAATCGATGTATTCCAAGAGTGTGGAAATAAAGAGAACTGAGATGGAA
TTCAAGAGAGAAGTGTCTCCTCCTCGTGTAGCAGCTTGAGAAGAGGCTTG
GGAGTGCAGCTTCTCAAACGAGACCGATGCTTGCTCAGGATGTCGACAGC
TGTGGCTTCCTTGTTTTTGCTAGCCATATTTTTAAATCAGGGTTGAACTG
ACAAAAATAATTTAAAGACGTTTACTTCCCTTGAACTTTGAACCTGTGAA
ATGCTTTACCTTGTTTACAATTTGGCAAAGTTGCAGTTTGTTCTTGTTTT
TAGTTTAGTTTTGTTTTGGTGTTTTGATACCTGTACTGTGTTCTTCACAG
ACCCTTTGTAGCGTGGTCAGGTCTGCTGTAACATTTCCCACCAACTCTCT
TGCTGTCCACATCAACAGCTAAATCATTTATTCATATGGATCTCTACCAT
CCCCATGCCTTGCCCAGGTCCAGTTCCATTTCTCTCATTCACAAGATGCT
TTGAAGGTTCTGATTTTCAACTGATCAAACTAATGCAAAAAAAAAAAGTA
TGTATTCTTCACTACTGAGTTTCTTCTTTGGAAACCATCACTATTGAGAG
ATGGGAAAAACCTGAATGTATAAGCATTTATTTGTCAATAAACTGCCTTT
TGTAAGGGGTTTTCACATAAAAAAAAAAAAAA
Human CBL-B mRNA sequence - var4
(public gi: 862408)

(SEQ ID NO: 58)

CTGGGTCCTGTGTGTGCCACAGGGGTGGGGTGTCCAGCGAGCGGTCTCCT
CCTCCTGCTAGTGCTGCTGCGGCGTCCCGCGGCCTCCCCGAGTCGGGCGG
GAGGGGAGAGCGGGTGTGGATTTGTCTTGACGGTAATTGTTGCGTTTCCA
CGTCTCGGAGGCCTGCGCGCTGGGTTGCTCCTTCTTCGGGAGCGAGCTGT
TCTCAGCGATCCCACTCCCAGCCGGGGCTCCCCACACACACTGGGCTGCG
TGCGTGTGGAGTGGGACCCGCGCACACGCGTGTCTCTGGACAGCTACGGC
GCCGAAAGAACTAAAATTCCAGATGGCAAACTCAATGAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAAGACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAACCAATGCCTCTAAAATCAACAATTGATTTAACTTGCAATGAT
TACATTTCAGTTTTTGAATTTGATATTTTTACCACGCTGTTTCAGCCTTG
GGGCTCTATTTTGCGGAATTGGAATTTCTTAGCTGTGACACATCCAGGTT
ACATCGCATTTCTCACATATGATCAAGTTAAAGCACGACTACAGAAATAT
AGCACCAAACCCGGAAGCTATATTTTCCGGTTAAGTTGCACTCCATTGGG
ACAGTGGGCCATTGGCTATGTGACTGGGGATGGGAATATCTTACAGACCA
TACCTCATAACAACCCCTTATTTCAAGCCCTGATTGATGGCAGCAGGGAA
GGATTTTATCTTTATCCTGATGGGAGGAGTTATAATCCTGATTTAACTGG
ATTATGTGAACCTACACCTCATGACCATATAAAAGTTACACAGGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGAGTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTCA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
CCCTGGATCTAATTCAGAAAGGCATAGTTAGATCTCCCTGTCGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATCTGGAAAGCGTCCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAACGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTCGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGGAAGATGATGATGAATACA
AGATTCCTTCATCCCACCCTGTTTCCCTGAATTCACAACCATCTCATTGT
CATAATGTAAAACCTCCTGTTCGGTCCTGTGATAATGGTCACTGTATGCT
GAATGGAACACATGGTCCATCTTCAGAGAAGAAATCAAACATCCCTGACT
TAAGCATATATTTAAAGGGAGATGTTTTTGATTCAGCCTCTGATCCCGTG
CCATTACCACCTGCCAGGCCTCCAACTCGGGACAATCCAAAGCATGGTTC
TTCACTCAACAGGACGCCCTCTGATTATGATCTTCTCATCCCTCCATTAG
GTTGAAACCTTTAAAAAAGTTTTGAACAACCCACCCCTCCTTCTTTTAAT
TTCAGAATTTTCAGAATTCAGAGTTCAGTATAACACAGACTCACTGGGTT
GTGAATTTGCCTGAAATTTGAATGGGTTCTCCAGGTGCCGGTGACTCCCA
AGTTCACGAGACCATTACTCCATGTAGATGATTAACGTAGTAGTGTAGTA
GTTGGGCATCAGTCAGGTTTTAAGCAAGTTGTTTTGTCCATACTAAATGT
AGTCTAAAAACACATGAGAGCTTTGTGCTCTAGTAGTTTTGAAGTGATGA
CTTGAAGTGTTGAGATTTTCTTTAAGTATAATAATTCTTAATAAATATGA
ACTTGCTTTTCTTGCAGCATGAGCACCAGTTCCACTTACGCTAATTAAAT
TATGCAAAATTAAATAGTTGTATGTAGAGAACTGATAATAAATTCTGTTT
TATTCTAATCATTACAACTGTAACACATTCAAAAAAAAAAA
Human CBL-B mRNA sequence - var5
(public gi: 862410)

(SEQ ID NO: 59)

CTGGGTCCTGTGTGTGCCACAGGGGTGGGGTGTCCAGCGAGCGGTCTCCT
CCTCCTGCTAGTCCTGCTGCGCCGTCCCGCCGCCTCCCCGAGTCGCGCGG
GAGGGGAGAGCGGGTGTGGATTTGTCTTGACCGTAATTGTTGCGTTTCCA
CGTCTCGGAGGCCTGCCCGCTGGGTTGCTCCTTCTTCGGGAGCGAGCTGT
TCTCACCGATCCCACTCCCAGCCGGGGCTCCCCACACACACTGCGCTGCG
TGCGTGTGGAGTGGGACCCGCGCACACGCGTGTCTCTGGACAGCTACCGC
GCCGAAAGAACTAAAATTCCAGATGGCAAACTCAATCAATGGCAGAAACC
CTGGTGGTCGAGGAGGAAATCCCCGAAAAGGTCGAATTTTGGGTATTATT
GATGCTATTCAGGATGCAGTTGGACCCCCTAAGCAAGCTGCCGCAGATCG
CAGGACCGTGGAGAACACTTGGAAGCTCATGGACAAAGTGGTAAGACTGT
GCCAAAATCCCAAACTTCAGTTGAAAAATAGCCCACCATATATACTTGAT
ATTTTGCCTGATACATATCAGCATTTACGACTTATATTGAGTAAATATGA
TGACAACCAGAAACTTGCCCAACTCAGTGAGAATGAGTACTTTAAAATCT
ACATTGATAGCCTTATGAAAAAGTCAAAACGGGCAATAAGACTCTTTAAA
GAAGGCAAGGAGAGAATGTATGAAGAACAGTCACAGGACAGACGAAATCT
CACAAAACTGTCCCTTATCTTCAGTCACATGCTGGCAGAAATCAAAGCAA
TCTTTCCCAATGGTCAATTCCAGGGAGATAACTTTCGTATCACAAAAGCA
GATGCTGCTGAATTCTGGAGAAAGTTTTTTGGAGACAAAACTATCGTACC
ATGGAAAGTATTCAGACAGTGCCTTCATGAGGTCCACCAGATTAGCTCTA
GCCTGGAAGCAATGGCTCTAAAATCAACAATTGATTTAACTTGCAATGAT
TACATTTCAGTTTTTGAATTTGATATTTTTACCAGGCTGTTTCAGCCTTG
GGGCTCTATTTTGCGGAATTGGAATTTCTTAGCTGTGACACATCCAGGTT
ACATGGCATTTCTCACATATGATGAAGTTAAAGCACGACTACAGAAATAT
AGCACCAAACCCGGAAGCTATATTTTCCGGTTAAGTTGCACTCGATTGGG
ACAGTGGGCCATTGGCTATGTGACTGGGGATGGGAATATCTTACAGACCA
TACCTCATAACAACCCCTTATTTCAAGCCCTGATTGATGGCAGCAGGGAA
GGATTTTATCTTTATCCTGATGGGACGAGTTATAATCCTGATTTAACTGG
ATTATGTGAACCTACACCTCATGACCATATAAAAGTTACACACGAACAAT
ATGAATTATATTGTGAAATGGGCTCCACTTTTCAGCTCTGTAAGATTTGT
GCAGAGAATGACAAAGATGTCAAGATTGAGCCTTGTGGGCATTTGATGTG
CACCTCTTGCCTTACGGCATGGCAGGACTCGGATGGTCAGGGCTGCCCTT
TCTGTCGTTGTGAAATAAAAGGAACTGAGCCCATAATCGTGGACCCCTTT
GATCCAAGAGATGAAGGCTCCAGGTGTTGCAGCATCATTGACCCCTTTGG
CATGCCGATGCTAGACTTGGACGACGATGATGATCGTGAGGAGTCCTTGA
TGATGAATCGGTTGGCAAACGTCCGAAAGTGCACTGACAGGCAGAACTCA
CCAGTCACATCACCAGGATCCTCTCCCCTTGCCCAGAGAAGAAAGCCACA
GCCTGACCCACTCCAGATCCCACATCTAAGCCTGCCACCCGTGCCTCCTC
GCCTGGATCTAATTCAGAAAGGCATAGTTAGATCTCCCTGTGGCAGCCCA
ACAGGTTCACCAAAGTCTTCTCCTTGCATGGTGAGAAAACAAGATAAACC
ACTCCCAGCACCACCTCCTCCCTTAAGAGATCCTCCTCCACCGCCACCTG
AAAGACCTCCACCAATCCCACCAGACAATAGACTGAGTAGACACATCCAT
CATGTGGAAAGCGTGCCTTCCAGAGACCCGCCAATGCCTCTTGAAGCATG
GTGCCCTCGGGATGTGTTTGGGACTAATCAGCTTGTGGGATGTCGACTCC
TAGGGGAGGGCTCTCCAAAACCTGGAATCACAGCGAGTTCAAATGTCAAT
GGAAGGCACAGTAGAGTGGGCTCTGACCCAGTGCTTATGCGGAAACACAG
ACGCCATGATTTGCCTTTAGAAGGAGCTAAGGTCTTTTCCAATGGTCACC
TTGGAAGTGAAGAATATGATGTTCCTCCCCGGCTTTCTCCTCCTCCTCCA
GTTACCACCCTCCTCCCTAGCATAAAGTGTACTGGTCCGTTAGCAAATTC
TCTTTCAGAGAAAACAAGAGACCCAGTAGAGCAAGATGATGATGAATACA
AGATTCCTTCATCCCACCCTGTTTCCCTGAATTCACAACCATCTCATTGT
CATAATGTAAAACCTCCTGTTCCGTCCTGTGATAATGGTCACTGTATGCT
GAATGGAACACATGGTCCATCTTCAGAGAAGAAATCAAACATCCCTGACT
TAAGCATATATTTAAACGGTACGTATAGAATATAATTTCCTTTGTGATGT
ACATCTTAATGGTCAGAATTTAAAGGCAAAATTTCATGCCATTGTACTGA
AAATACATTAAGGTTTTGTGTTATCCTCTAGGAGATGTTTTTGATTCAGC
CTCTGATCCCGTGCCATTACCACCTGCCAGGCCTCCAACTCGGGACAATC
CAAAGCATGGTTCTTCACTCAACAGGACGCCCTCTGATTATGATCTTCTC
ATCCCTCCATTAGGTTGAAACCTTTAAAAAAGTTTTGAACAACCCACCCC
TCCTTCTTTTAATTTCAGAATTTTCAGAATTCAGAGTTCAGTATAACACA
GACTCACTGGGTTGTGAATTTGCCTGAAATTTGAATCGGTTCTCCAGGTG
CCGGTGACTCCCAAGTTCACGAGACCATTACTCCATGTAGATGATTAAGG
TAGTAGTGTAGTACTTGGGCATCAGTCAGGTTTTAAGCAAGTTGTTTTGT
CCATACTAAATGTAGTCTAAAAACACATGAGAGCTTTGTGCTCTAGTAGT
TTTGAAGTGATGACTTGAAGTGTTGAGATTTTCTTTAAGTATAATAATTC
TTAATAAATATGAACTTGCTTTTCTTGCAGCATGAGCACCAGTTCCACTT
ACGCTAATTAAATTATGCAAAATTAAATAGTTGTATGTAGAGAACTGATA
ATAAATTCTGTTTTATTCTAATCATTACAACTGTAACACATTCAAAAAAA
AAAA
Human CBL-B mRNA sequence - var6
(public gi: 21753192)

(SEQ ID NO: 60)

AGTGCTGCTGCGGCGTCCCGCGGCCTCCCCGAGTCGGGCGGGACGGGAGA
GCCGGTGTGGATTTGTCTTGACGGTAATTGTTGCGTTTCCACGTCTCGGA
GGCCTGCCCGCTGGGTTGCTCCTTCTTCGGGAGCGAGCTGTTCTCAGCGA
TCCCACTCCCAGCCGGGGCTCCCCACACACACTGGGCTGCGTGCGTGTGG
AGTGGGACCCGCGCACACGCGTGTCTCTGGACAGCTACGGCGCCGAAAGA
ACTAAAATTCCAGATGGCAAACTCAATGAATGGCAGAAACCCTGGTCGTC
GACGAGGAAATCCCCGAAAAGGTCGAATTTTGGGTATTATTGATGCTATT
CAGGATGCAGTTGGACCCCCTAACCAAGCTGCCGCAGATCGCAAAACCTG
GAATCACAGCGAGTTCAAATGTCAATGGAAGGCACAGTAGAGTGCGCTCT
GACCCAGTGCTTATGCCGAAACACAGACGCCATGATTTGCCTTTAGAAGG
AGCTAAGGTCTTTTCCAATGGTCACCTTGGAAGTGAAGAATATGATGTTC
CTCCCCGGCTTTCTCCTCCTCCTCCAGTTACCACCCTCCTCCCTAGCATA
AAGTGTACTGGTCCGTTAGCAAATTCTCTTTCAGAGAAAACAAGAGACCC
AGTAGAGGAAGATGATOATGAATACAAGATTCCTTCATCCCACCCTGTTT
CCCTGAATTCACAACCATCTCATTGTCATAATGTAAAACCTCCTGTTCGG
TCTTGTGATAATCGTCACTGTATGCTGAATGGAACACATGGTCCATCTTC
AGAGAAGAAATCAAACATCCCTGACTTAAGCATATATTTAAAGGGAGATG
TTTTTGATTCAGCCTCTGATCCCGTGCCATTACCACCTGCCAGGCCTCCA
ACTCGGGACAATCCAAAGCATGGTTCTTCACTCAACAGGACGCCCTCTGA
TTATGATCTTCTCATCCCTCCATTAGGTGAAGATGCTTTTGATGCCCTCC
CTCCATCTCTCCCACCTCCCCCACCTCCTGCAAGGCATAGTCTCATTGAA
CATTCAAAACCTCCTGGCTCCAGTAGCCGGCCATCCTCAGGACAGGATCT
TTTTCTTCTTCCTTCAGATCCCTTTGTTGATCTAGCAAGTGGCCAAGTTC
CTTTGCCTCCTGCTAGAAGGTTACCAGGTGAAAATGTCAAAACTAACAGA
ACATCACAGGACTATGATCAGCTTCCTTCATGTTCAGATGGTTCACAGGC
ATCAGCCAGACCCCCTAAACCACGACCGCGCAGGACTGCACCAGAAATTC
ACCACAGAAAACCCCATGGGCCTGAGGCGGCATTGGAAAATGTCGATCCA
AAAATTGCAAAACTCATGGGAGAGGGTTATGCCTTTGAAGAGGTGAAGAG
AGCCTTAGAGATAGCCCAGAATAATGTCGAAGTTGCCCGGAGCATCCTCC
GAGAATTTGCCTTCCCTCCTCCAGTATCCCCACGTCTAAATCTATAGCAG
CCAGAACTGTAGACACCAAAATGGAAAGCAATCGATGTATTCCAAGAGTG
TGGAAATAAAGAGAACTGAGATGGAATTCAAGAGAGAAGTGTCTCCTCCT
CGTGTAGCAGCTTGAGAAGAGGCTTGGGAGTGCAGCTTCTCAAAGGAGAC
CGATGCTTGCTCAGGATGTCGACAGCTGTGGCTTCCTTGTTTTTGCTAGC
CATATTTTTAAATCAGGGTTGAACTGACAAAAATAATTTAAAGACGTTTA
CTTCCCTTGAACTTTGAACCTGTGAAATGCTTTACCTTGTTTACAGTTTG
GCAAAGTTGCAGTTTGTTCTTGTTTTTAGTTTAGTTTTGTTTTGGTGTTT
TGTACCTGTACTGTGTTCTTCACAGACCCTTTGTAGCGTGGTCAGGTCTG
CTGTAACATTTCCCACCAACTCTCTTGCTGTCCACATCAACAGCTAAATC
ATTTATTCATATGGATCTCTACCATCCCCATGCCTTGCCCAGGTCCAGTT
CCATTTCTCTCATTCACAAGATGCTTTGAAGGTTCTGATTTTCAACTGAT
CAAACTAATGCAAAAAAAAAAAAAAAAAAAAAAAAAAG
Human Cbl-b mRNA sequence - var7

(SEQ ID NO: 61)

CGTNTTTGGNANNCACTACAGGGGATGTTTAATACACACTCACAATGCCC
ATGATGTNTATAACTATCTATTCNATGATGTAAGATACCCCACTCAAACC
CATAAAAAAGAGCATCTTTAATACGACTCACTATANGGCGAGCGCACGCC
ATGGCAGGTACCCATACGACGTACCAGATTACGCTCATATGGCCATGGAG
GCCAGNGAATTCCACCCAAGCNGTGGTATCAACGCANACTGGACTCTGAC
CCANTGCTTATGCGGAAACACAGACGCCATGATTTGCCTTTAGAAGGAGC
TAAGGTCTCTTCCAATGGTCACCTTGGAAGTGAAGAATATGATGTTCCTC
CCCGGCTTTCTCCTCCTCCTCCAGTTACCACCCTNCTCCCTAGCATAAAG
TGTACTGGTCCGTTAGCAAATTCTCTTTCAGAGAAAACAAGAGACCCAGT
AGAGGAAGATGATGATGAATACAAGATTCCTTCATCCCACCCTGTTTCCC
TGAATTCACAACCATCTCATTGTCATAATGTAAAACCTCCTGTTCGGTCT
TGTGATAATGGTCACTGTATGCTGAATGGAACACATGGTCCATCTTCAGA
GAAGAAATCAAACATCCCTGACTTAAGCATATATTTAAAGGGTGAAGATG
CTTTTGATGCCCTCCCTCCATCTCTCCCACCTCCCCCACCTCCTGCAAGC
CATAGTCTCATTGAACATTCAAAACCTCCTGGCTCCAGTAGCCGGCCATC
CTCAGGACAGGATCTTTTTCTTCTTCCTTCAGATCCCTTTGTTGATCTAG
CAAGTGGCCAAGTTCCTTTGCCTCCCGCTAGAAGGTTACCAGGTGAAAAT
GTCAAAACTAACAGGACATCACAGGACTATGATCAGCTTCCTTCATGTTC
AGATGGTTCACAGGCACCAGCCAGACCCCCTAAACCACGACCGCGCAGGA
CTGCACCAGAAATTCACCACAGAAAACCCCATGGGCCTGAGGCGGCATTG
GAAAATGTCGATGCAAAAATTGCAAAACTCATGGGAGAGGGTTATGCCTT
TGAAGAGGTGAAGAGAGCCTTAGAGATAGCCCAGAATAATGTCGAAGTTG
CCCGGAGCATCCTCCGAGAATTTGCCTTCCCTCCTCCAGTATCCCCACGT
CTAAATCTATAGCAGCCAGAACTGTAGACACCAAAATGGAAAGCAATCGA
TGTATTCCAAGAGTGTGGAAATAAAGAGAACTGAGATGGAATTCAAGAGA
GAAGTGTCTCCTCCTCGTGTAGCAGCTTGAGAAGAGGCTTGGGAGTGCAG
CTTCTCAAAGAAAACCGATGCTTGCTCAGGATGTCNACAGCTGNGGNCTN
CCTTGTTTTTGCTAGCCATTTTTTTAAATNAGGGTTGAACTNGANAAAAN
TATTTAAAAACGTTTACCTCCCTTGAACTTTGAACCTGGGAAAGNC
Human Cbl-b Protein sequence - var5

(SEQ ID NO: 83)

MRKHRRHDLPLEGAKVSSNGHLGSEEYDVPPRLSPPPPVTTLLPSIKCTG
PLANSLSEKTRDPVEEDDDEYKIPSSHPVSLNSQPSHCHNVKPPVRSCDN
GHCMLNGTHGPSSEKKSNIPDLSIYLKGEDAFDALPPSLPPPPPPARHSL
IEHSKPPGSSSRPSSGQDLFLLPSDPFVDLASGQVPLPPARRLPGENVKT
NRTSQDYDQLPSCSDGSQAPARPPKPRPRRTAPEIHHRKPHGPEAALEVD
AKIAKLMGEGYAFEEVKRALEIAQNNVEVARSILREFAFPPPVSPRLNL
Human cbl-B clone3Gd114 (partial sequence)

(SEQ ID NO: 62)

ACTCTGACCCAGTGCTTATGCGGAAACACAGACGCCATGATTTGCCTTTA
GAAGGAGCTAAGGTCTCTTCCAATGGTCACCTTGGAAGTGAAGAATATGA
TGTTCCTCCCCGGCTTTCTCCTCCTCCTCCAGTTACCACCCTCCTCCCTA
GCATAAAGTGTACTGGTCCGTTAGCAAATTCTCTTTCAGAGAAAACAAGA
GACCCAGTAGAGGAAGATGATGATGAATACAAGATTCCTTCATCCCACCC
TGTTTCCCTGAATTCACAACCATCTCATTGTCATAATGTAAAACCTCCTG
TTCGGTCTTGTGATAATGGTCACTGTATGCTGAATGGAACACATGGTCCA
TCTTCAGAGAAGAAATCAAACATCCCTGACTTAAGCATATATTTAAAGGG
TGAAGATGCTTTTGATGCCCTCCCTCCATCTCTCCCACCTCCCCCACCTC
CTGCAAGGCATAGTCTCATTGAACATTCAAAACCTCCTGGCTCCAGTAGC
CGGCCATCCTCAGGACAGGATCTTTTTCTTCTTCCTTCAGATCCCTTTGT
TGATCTAGCAAGTGGCCAAGTTCCTTTGCCTCCCGCTAGAAGGTTACCAG
GTGAAAATGTCAAAACTAACAGGACATCACAGGACTATGATCAGCTTCCT
TCATGTTCAGATGGTTCACAGGCACCAGCCAGACCCCCTAAACCACGACC
GCGCAGGACTGCACCAGAAATTCACCACAGAAAACCCCATGGGCCTGAGG
CGGCATTGGAAAATGTCGATGCAAAAATTGCAAAACTCATGGGAGAGGGT
TATGCCTTTGAAGAGGTGAAGAGAGCCTTAGAGATAGCCCAGAATAATGT
CGAAGTTGCCCGGAGCATCCTCCGAGAATTTGCCTTCCCTCCTCCAGTAT
CCCCACGTCTAAATCTATAGCAGCCAGAACTGTAGACACCAAAATGGAAA
GCAATCGATGTATTCCAAGAGTGTGGAAATAAAGAGAACTGAGATGGAAT
TCAAGAGAGAAGTGTCTCCTCCTCGTGTAGCAGCTTGAGAAGAGGCTTGG
GAGTGCAGCTTCTCAAAGAAAACCGATGCTTGCTCAGGATGTCGACAGCT
GTGGCTTCCTTGTTTTTGCTAGCCATTTTTTTAAATCAGGGTTGAACTGG
AAAAAATTATTTAAAAACGTTTACCTCCCTTGAACTTTGAACCTGGGAAA
GGC
Human CblB protein in 3Gd114 Translation of cbl-B
clone3Gd114 starting at base pair 3

(SEQ ID NO: 84)

SDPVLMRKHRRHDLPLEGAKVSSNGHLGSEEYDVPPRLSPPPPVTTLLPS
IKCTGPLANSLSEKTRDPVEEDDDEYKIPSSHPVSLNSQPSHCHNVKPPV
RSCDNGHCMLNGTHGPSSEKKSNIPDLSIYLKGEDAFDALPPSLPPPPPP
ARHSLIEHSKPPGSSSRPSSGQDLFLLPSDPFVDLASGQVPLPPARRLPG
ENVKTNRTSQDYDQLPSCSDGSQAPARPPKPRPRRTAPEIHHRKPHGPEA
Human CBL-B Protein sequence - var1
(public gi: 4757920)

(SEQ ID NO: 85)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFOGDNFRITKADAAEFWRKFFGDKTIVPWKVFROC
LHEVHQISSSLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRXQDKPLPAPPPP
LRDPPPPPPERPPPIPPDNRLSRHIHHVESVPSRDPPMPLEAWCPRDVFG
TNQLVGCRLLGEGSPKPGITASSNVNGRMSRVGSDPVLMRKHRRHDLPLE
GAKVFSNGMLGSEEYDVPPRLSPPPPVTTLLPSIKCTGPLANSLSEKTRD
PVEEDDDEYKIPSSHPVSLNSQPSHCHNVKPPVRSCDNGHCMLNGTHCPS
SEKKSNIPDLSIYLKCTYRI
Human CBL-B Protein sequence - var2
(public gi: 23273909)

(SEQ ID NO: 86)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERNYEEQSQDRRNLTKLSLIF
SHMLAEIKAIFPNGQFQGDNFRITKADAAEFWRKFFGDKTIVPWKVFRQC
LMEVHQISSGLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRHIHHVESVPSKDPPMPLEAWCPRDVFG
TNQLVGCRLLGEGSPKPGITASSNVNGRHSRVGSDPVLMRKHRRHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLGEDAFDALPPSLPPPPPPARHSLIEHSKPPGSSSRPSSGQD
LFLLPSDPPVDLASGQVPLPPARRLPGENVKTNRTSQDYDQLPSCSDGSQ
APARPPKPRPRRTAPEIHHRKPHGPEAALENVDAKIAKLMGEGYAFEEVK
RALEIAQNNVEVARSILREFAFPPPVSPRLNL
Human CBL-B Protein sequence - var3
(public gi: 862407)

(SEQ ID NO: 87)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKIAQ
LSENEYFKIYIDSLMKKSKRAIRLFKEGKERMYEEQSQDRRNLTKLSLIP
SHMLAEIKAIFPNGQFQGDNFRITKADAAEFWRKFPGDKTIVPWKVFRQC
LHEVHQISSSLEAMALKSTIDLTCNDYISVFEFDIPTRLFQPWGSILRNW
NFLAVTMPGYMAFLTYDEVKARLQKYSTKPGSYIFRLSCTRLCQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRHIHHVESVPSRDPPMPLEAWCPRDVFG
TNQLVGCRLLGEGSPKPGITASSNVNGRHSRVGSDPVLMRKMRRHDLPLE
GAKVFSNGHLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLGEDAPDALPPSLPPPPPPAPHSLIEHSKPPGSSSRPSSGQD
LFLLPSDPFVDLASGQVPLPPARRLPGENVKTNRTSQDYDQLPSCSDGSQ
APARPPKPRPRRTAPEIHHRKPHGPEAALENVDAKIAKLMGEGYAFEEVK
RALEIAQNNVEVARSILREFAFPPPVSPRLNL
Human CBL-B Protein sequence - var4
(public gi: 862409)

(SEQ ID NO: 88)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERNYEEQSQDRRNLTKLSLIF
SHMLAEIKAIFPNGQFQGDNFRITKADAAEFWRKFFGDKTIVPWKVFRQC
LHEVHQISSSLEAMALKSTIDLTCNDYISVFEFDIFTRLFQPWGSILRNW
NFLAVTHPGYMAFLTYDEVKARLQKYSTKPGSYIFRLSCTRLGQWAIGYV
TGDGNILQTIPHNKPLFQALIDGSREGFYLYPDGRSYNPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFGMPMLDLD
DDDDREESLMMNRLANVRKCTDRQNSPVTSPGSSPLAQRRKPQPDPLQIP
HLSLPPVPPRLDLIQKGIVRSPCGSPTGSPKSSPCMVRKQDKPLPAPPPP
LRDPPPPPPERPPPIPPDNRLSRRIHHVESVPSRDPPMPLEAWCPRDVFG
TNQLVGCRLLGEGSPKPGITASSNVNGRMSRVGSDPVLMRKHRRHDLPLE
GAKVFSNGMLGSEEYDVPPRLSPPPPVTTLLPSIKCTGPLANSLSEKTRD
PVEEDDDEYKIPSSHPVSLNSQPSHCHNVKPPVRSCDNGHCMLNGTHGPS
SEKKSNIPDLSIYLKGDVFDSASDPVPLPPARPPTRDNPKHGSSLNRTPS
DYDLLIPPLG
Rat CBL-B MRNA sequence
(public gi: 21886623)

(SEQ ID NO: 147)

CGGGCGGGCGTGGAGCTGTCTGCACGAAAGGACTAAGATTCCAGATGGCA
AATTCTATGAATGGCAGAAATCCTGGTGGTCGAGGAGGAAACCCCCGCAA
AGGTCGAATTTTGGGGATTATTGATGCCATTCACGATGCAGTTGGACCCC
CAAAGCAAGCTGCAGCTGACCGCAGGACAGTGGAGAAGACTTGGAAACTC
ATGGACAAAGTGGTAAGACTGTGCCAAAATCCGAAACTTCAGTTGAAAAA
CAGCCCACCATATATCCTCGACATTTTACCTGATACGTATCAGCATTTGC
GGCTTATATTGAGTAAGTATGACGACAACCAGAAGCTGGCTCAACTGAGC
GAGAATGAGTACTTTAAAATCTACATCGACAGTCTCATGAAGAAGTCAAA
GCGAGCGATCCGGCTCTTCAAAGAAGGCAAGGAGAGGATGTACGAGGAGC
AGTCGCAGGACAGACCGAATCTCACAAAGCTGTCCCTTATCTTCAGTCAC
ATGCTGGCAGAAATCAAGGCGATCTTTCCCAATGGCCAGTTCCAGGGAGA
TAACTTCCGGATCACCAAAGCAGATGCTGCCGAATTCTCGAGGAAGTTTT
TTGGAGACAAAACTATCGTACCATGGAAAGTCTTCAGACAGTGCVTGCAT
GACGTCCATCAGATCAGCTCTGGCCTGGAGGCCATGGCTCTGAAGTCAAC
CATTGACTTAACTTGTAATGATTACATCTCCGTGTTTGAATTTGATATTT
TTACCAGGCTATTTCAGCCCTGGGGCTCTATTTTACCGAATTCGAACTTC
TTAGCTGTGACACACCCGGGGTACATGGCATTTCTCACATATGATGAAGT
TAAAGCTCGACTACAGAAATACAGCACCAAGCCTGGAAGCTACATTTTCC
GGTTAAGCTGCACTCGGCTGGGACAATGGGCCATTGGCTATGTGACTGGG
GACGGCAATATCCTACAGACCATACCTCATAACAAGCCCCTGTTCCAAGC
CCTGATTGATGGTAGCAGGGAAGGCTTTTACCTTTATCCAGATGGACGAA
GCTATAACCCTGATTTAACCGGATTATGTGAACCTACACCTCATGATCAT
ATAAAAGTTACACAGGAGCAATATGAACTGTATTGTGAAATGGGCTCCAC
TTTTCAGCTGTGCAAGATCTGTGCAGAGAATGACAAAGATGTCAAGATCG
AGCCTTGTGGGCATCTCATGTGCACTTCCTGCCTTACCCCGTCGCAGGAG
TCTGATGGCCAAGGCTGCCCCTTCTGTCGCTGTGAGATAAAAGGAACCGA
ACCTATCATCGTGGATCCCTTTGACCCCAGAGACGAAGGCTCCAGGTGCT
GCAGCATCATCGACCCTTTCAGCATCCCCATGCTCGACTTGGATGATGAC
GATGATCGAGAGGAGTCTCTGATGATGAACCGGCTGGCGAGTGTTCGCAA
GTGCACAGACAGGCAGAACTCGCCAGTCACATCGCCAGGATCCTCACCCC
TTGCCCAGAGAAGAAAGCCTCAGCCAGACCCTCTCCAGATCCCCCACCTC
AGCCTGCCACCAGTGCCTCCCCGCCTGGACCTCATTCAGAAAGGCATCGT
GCGCTCTCCCTGTGGCAGCCCCACGGGCTCCCCGAAGTCTTCTCCATGCA
TGGTTAGAAAACAACACAAACCACTCCCAGCACCCCCTCCTCCCTTGCGA
GATCCTCCGCCTCCACCAGAGCGGCCTCCGCCAATCCCGCCTGACAGTAG
ACTGAGCAGACACTTCCACCACGGAGAGAGTGTGCCTTCCAGGGACCAGC
CAATGCCTCTTGAAGCCTGGTGCCCTCGGGATGCCTTCGGGACTAATCAG
GTGATGGGATGTCGCATCCTAGGGGATCGCTCTCCAAAGCCTGGCGTCAC
AGCAAACTCCAACTTAAATGGACGTCACAGTCGAATGGGCTCTGACCAGG
TTCTTATGAGGAAACACAGACGCCACGATTTGCCTTCAGAAGGCGCCAAG
GTCTTTTCCAATGGACACCTTGCCCCTGAAGAATACGACGTTCCTCCTCG
GCTTTCCCCTCCTCCTCCAGTCACTGCCCTTCTCCCTAGCATAAAGTGTA
CTGGTCCAATAGCAAATTGTCTCTCCCAGAAAACAAGAGACACAGTAGAA
GAAGATGATGATGAATACAAGATTCCTTCATCCCATCCTGTTTCCCTGAA
TTCACAACCATCTCATTGTCATAATGTCAAACCTCCTGTTCGGTCTTGTG
ATAATGGTCACTGTATACTGAATGGAACTCATGGTACGCCTTCAGAGATG
AAGAAATCAAACATCCCAGATTTAGGCATCTATTTGAAGGGTGAAGATGC
TTTTGATGCCCTCCCCCCATCCCTTCCTCCTCCCCCACCTCCTGCAAGAC
ATAGTCTCATCGAGCATTCAAAACCTCCAGGCTCCAGTAGCCGGCCTTCC
TCAGGACAGGACCTTTTCCTTCTTCCTTCAGATCCCTTTTTTGACCCAGC
AAGTGGCCAAGTTCCATTGCCTCCGGCCAGGAGAGCACCAGGAGATGGTG
TCAAATCCAACAGAGCCTCCCAGGACTATGACCAGCTCCCTTCATCTTCC
GATGGTTCGCAAGCACCAGCTAGACCCCCCAAACCACGACCCCGAAGGAC
TGCACCAGAAATTCATCACAGAAAGCCCCATGGGCCCGAGGCGGCACTCG
AAAATGTGGATGCGAAAATTGCAAAACTCATGGGAGAGGGGTATGCCTTT
GAAGAGGTGAAGAGAGCCTTAGAGATCGCCCAGAATAACCTGGAAGTGGC
CAGGAGCATACTTCGAGAATTCGCCTTCCCTCCTCCCGTCTCGCCACGTC
TCAATCTATAGCAGCCCAGACTGCAAACACCAAAGGGTAAAACAGTTAAC
AAATATTCCAGGAGTATGGGACAGAAGGACTGAGAGGGAATGCAGGAGCC
ATCGTGTCTTTTCATGTGGCGTCTCCAGAAGGCAGCCTTGAGTCCAGCTT
CTCTGGTACCACAGCTCCCTGAGGATGCCCACGCTGCAGCTTCTGTGTTT
GTGCTAGCCATACTTTTAAATCAGGGTTGAACTGAGAAAATAATTTAAAG
ACGTTTACTCCCCCTTGAACTTTGAATCTGTGAAATGCTTTCCTTGTTTA
CACGTTGGCAGAATTGCAGTTTGTCTCTGTTTTTGATTCCTGTACTCTGT
TCCTGACAGGCCCTTGGCAGAGTTGGTCACGTCTGCTGTAAGTTTGTCCA
TGCCCACCCTGCTGCCCACATTGGCAGCTAAAGCATCTCTTCGTGTTGCT
GTCTATCCCGGCCCCACCTCATGTGTCCACGTCCAGTTCATTTCTCTCAT
TCACACAGCATGCTAGTCTGAGG
Rat CBL-B Protein sequence
(public gi: 21886624)

(SEQ ID NO: 148)

MANSMNGRNPGGRGGNPRKGRILGIIDAIQDAVGPPKQAAADRRTVEKTW
KLMDKVVRLCQNPKLQLKNSPPYILDILPDTYQHLRLILSKYDDNQKLAQ
LSENEYFKIYIDSLMKKSKRAIRLFKEGKERMYEEQSQDRRNLTKLSLIF
SHMLAEIKAIFPNGQFQGDNFRITKADAAEFWRKFFGDKTIVPWKVFRQC
LHEVHQISSGLEAMALKSTIDLTCNDYISVFEFDIFTRLFQPWGSILRNW
NFLAVTHPGYMAFLTYDEVKARLQKYSTKPGSYIFRLSCTRLGQWAIGYV
TCDGNILQTIPHNKPLFQALIDGSREGFYLYPDGRSYIPDLTGLCEPTPH
DHIKVTQEQYELYCEMGSTFQLCKICAENDKDVKIEPCGHLMCTSCLTAW
QESDGQGCPFCRCEIKGTEPIIVDPFDPRDEGSRCCSIIDPFSIPMLDLD
DDDDREESLMMNRLASVRKCTDRQNSPVTSPGSSPLAQRRKPQPDPLQIP
MLSLPPVPPRLDLIQKGIVRSPCGSPTGSPKSSPCMVRKQDKPLPAPPPP
LRDPPPPPERPPPIPPDSRLSRHFHHGESVPSRDQPMPLEAWCPRDAFGT
NQVMGCRILGDGSPKPGVTANSNLNGRHSRMGSDQVLMRKMRRHDLPSEG
AKVFSNGHLAPEEYDVPPRLSPPPPVTALLPSIKCTGPIANCLSEKTRDT
VEEDDDEYKIPSSHPVSLNSQPSHCMNVKPPVRSCDNGHCILNGTHGTPS
EMKKSNIPDLGIYLKGEDAPDALPPSLPPPPPPARHSLIEHSKPPGSSSR
PSSGQDLFLLPSDPFFDPASGQVPLPPARRAPGDGVKSNRASQDYDQLPS
SSDGSQAPARPPKPRPRRTAPEIHMRKPHGPEAALENVDAKIAKLMGEGY
AFEEVKRALEIAQNNLEVARSILREFAFPPPVSPRLNL
Mouse GBL-B mRNA sequence
(public gi: 26324665)

(SEQ ID NO: 149)

GACTCCCTGGGCTGCGAGCGCCGGCGGTCGTTGCCGGAGAGGCCCCTCCT
TCTCGCCCGGCTCCATTCCCTCGCTCGCGGCCGAGCGGGCTCCCGACCCT
CCGCTGGCCATGGCCGGCAACGTGAAGAAGAGCTCGGGCGCCGGCGGCGG
CGGCTCTGGGGGCTCGGGAGCGGGCGGCCTGATCGGGCTCATGAAGGACG
CCTTCCAGCCGCACCACCACCACCACCACCTCAGCCCGCACCCTCCCTGC
ACGGTGGACAAGAAGATGGTGGAGAAGTGCTGGAAGCTCATGGACAACGT
GGTGCGGTTGTGTCAAAACCCAAAGCTGGCGCTCAAGAACAGCCCGCCTT
ATATCTTAGACCTGCTGCCTGACACCTACCAGCACCTCCGCACTGTCTTG
TCAAGATATGAGGGGAAGATGGAGACGCTTCGACAAAATGAGTATTTCAG
GGTCTTCATGGAAAATTTGATGAAGAAAACTAAGCAGACTATCAGCCTCT
TCAAGGAGGGAAAAGAAAGGATGTATGAGGAGAATTCCCAGCCTAGGCGA
AACCTGACCAAATTATCCCTGATCTTCAGCCACATGCTGGCAGAACTGAA
AGGCATCTTTCCGAGCGGACTCTTCCAAGGAGACACTTTCCGGATTACTA
AAGCTGATGCTGCCGAATTTTGGAGAAAACCTTTTGGTGAAAAGACGATA
GTCCCGTGGAAGAGCTTTCGACAGGCCCTGCATGAAGTGCATCCCATCAG
TTCTGGGCTGGACGCCATGGCTCTGAAGTCCACTATTGATCTGACCTGCA
ATGATTATATTTCTGTCTTTGAATTTGATATTTTTACACGGCTGTTTCAC
CCCTGGTCCTCTTTGCTCAGAAATTGGAACAGCCTTGCTGTAACTCACCC
TGGTTACATGGCTTTCCTGACATACGATGAAGTGAAAGCGCGCCTGCAGA
AGTTCATCCACAAACCTCGCAGTTACATCTTTCGGCTGAGCTGTACTCGT
TTGGGTCAGTGGGCTATTGGGTATGTTACTCCCGATGGGAACATTCTGCA
GACAATCCCACACAATAAACCGCTCTTCCAAGCACTGATTGATGGCTTCA
GGGAAGGCTTCTATTTGTTTCCTGATGGACGAAATCAAAATCCTGACCTG
ACAGGTTTATGTGAACCAACTCCTCAAGATCATATCAAAGTAACCCAGGA
ACAATATGAATTATACTGTGAAATGGGCTCCACATTTCAACTGTGTAAGA
TATGTGCTGAGAATGATAAGGATGTGAAGATTGAGCCCTGTGGACACCTC
ATGTGCACATCCTGCCTCACGTCGTGGCAGGAATCAGAAGGTCAGGGCTG
TCCTTTTTGCCGATGTGAAATCAAAGGTACTGAGCCCATCGTGGTGGATC
CGTTTGACCCCAGAGGCAGTGGCAGCCTATTAAGGCAAGGAGCAGAAGGT
GCTCCTTCCCCAAATTACGACGATGATGATGATGAACGAGCTGATGATTC
TCTCTTCATGATGAAGGAGTTGGCAGGTGCCAAGGTGGAAAGGCCTTCCT
CTCCATTCTCCATGGCCCCACAAGCTTCCCTTCCTCCAGTGCCACCAAGA
CTTGACCTTCTACAGCAGCGAGCACCTGTTCCTGCCAGCACTTCAGTTCT
GGGGACTGCTTCCAAGGCTGCTTCTGGCTCCCTTCATAAAGACAAACCAT
TGCCAATACCTCCCACACTTCGAGATCTTCCACCACCACCCCCTCCAGAC
CGGCCTTACTCTGTTGGAGCAGAAACAAGGCCTCAGAGACGCCCTCTGCC
TTGTACACCAGGCGATTGTCCATCTAGAGACAAACTGCCCCCTGTCCCTT
CTAGCCGCCCAGGGGACTCGTGOTTGTCTCGGCCAATCCCTAAAGTACCA
GTAGCTACTCCAAACCCTGGTGATCCTTGGAATGGGAGAGAATTGACCAA
TCGGCACTCGCTTCCATTCTCATTGCCCTCACAAATGGAACCCAGAGCAG
ATGTCCCTAGGCTTGGAAGCACATTTAGTCTGGATACCTCTATGACTATG
AATAGCAGCCCAGTAGCAGGTCCAGAGAGTGAGCACCCAAAGATCAAGCC
TTCCTCGTCTGCCAACGCCATTTACTCTCTGGCTGCCAGGCCTCTTCCTA
TGCCAAAACTGCCACCTGGGGAGCAAGGGGAAAGTGAAGAGGACACAGAA
TATATGACTCCCACATCTAGGCCTGTAGGGGTTCAGAAGCCAGAGCCCAA
ACCGCCGTTAGAGGCAACCCAGAGTTCACGAGCATGTGACTGTGACCAGC
AGATCGACAGCTGTACCTATGAAGCGATGTATAACATCCAGTCCCAAGCA
CTGTCTGTAGCAGAAAACACCGCCTCTGGGGAAGGGAATCTGGCCACAGC
TCACACCAGTACTGGCCCTGAGGAATCCGAAAACGAGGATGATGGCTATG
ATGTGCCTAAGCCACCCGTGCCAGCTGTACTGGCCCGCCGGACCCTGTCT
GACATCTCCAATGCCAGCTCCTCCTTTGGCTGGTTGTCTTTGGATGGTGA
CCCTACAAACTTCAATGAGGGTTCCCAAGTTCCTGAGCGGCCCCCCAAAC
CATTCCCTCGGAGAATCAACTCAGAACGAAAAGCCAGTAGCTATCAACAA
GGCGGAGGTGCCACTGCTAACCCTGTGGCCACAGCACCCTCACCGCAGCT
CTCAAGTGAGATTGAACGCCTCATGAGTCAGGGCTATTCCTACCAGGACA
TTCAGAAAGCTTTGGTCATTGCCCACAACAACATTGAGATGGCTAAAAAC
ATCCTCCGGGAATTTGTTTCTATTTCTTCTCCTGCTCACGTAGCCACCTA
GCACATCTCTCCCTGCCACGGCTTCAGAGGACCCATGAGCCAGGCTCTTA
CTCAAGGACCACCTAGGAAAGCAGTGGCTTCTTTTGGGACGTCACAGTAA
GGTCCTGCCTTTCCTGTGGGGATCGACACATATGGTTCCAAGATTTCAAA
GCAGTGGAATGAAAATGGAGCAGCTGATGTGTTTCATTGTTGTATTGGTC
TTAAGAGTGTTTTTGTAGTCCTGCAGTCTCCAGTAGGAGAGAGTGGGTTT
TTATTAAATGGTAACCTACCCCAGAAACAGC
Mouse CBL-B Protein sequence
(public gi: 26324666)

(SEQ ID NO: 150)

MAGNVKKSSGAGGGGSGGSGAGGLIGLMKDAFQPHHMHHHLSPHPPCTVD
KKMVEKCWKLMDKVVRLCQNPKLALKNSPPYILDLLPDTYQHLRTVLSRY
EGKNETLGENEYFRVFMENLMKKTKQTISLFKEGKERMYEENSQPRRNLT
KLSLIFSMMLAELKGIFPSGLFQGDTFRITKADAAEFWRKAFGEKTIVPW
KSFRQALHEVHPISSGLDAMALKSTIDLTCNDYISVFEFDIFTRLFQPWS
SLLRNWNSLAVTHPGYMAFLTYDEVKARLQKFIHKPGSYIFRLSCTRLGQ
WAIGYVTADGNILQTIPHNKPLFQALIDGFREGFYLFPDGRNQNPDLTGL
CEPTPQDHIKVTQEQYELYCEMGSTFQLCKICAENDKDVKIEPCGHLMCT
SCLTSWQESEGQGCPFCRCEIKGTEPIVVDPFDPRGSGSLLRQGAEGAPS
PNYDDDDDERADDSLFMMKELAGAKVERPSSPFSMAPQASLPPVPPRLDL
LQQRAPVPASTSVLGTASKAASGSLHKDKPLPIPPTLRDLPPPPPPDRPY
SVGAETRPQRRPLPCTPGDCPSRDKLPPVPSSRPGDSWLSRPIPKVPVAT
PNPGDPWNGRELTNRHSLPFSLPSQMEPRADVPRLGSTFSLDTSMTNNSS
PVAGPESEHPKIKPSSSANAIYSLAARPLPMPKLPPGEQGESEEDTEYMT
PTSRPVGVQKPEPKRPLEATQSSRACDCDQQIDSCTYEANYNIQSQALSV
AENSASGEGNLATAHTSTGPEESENEDDGYDVPKPPVPAVLARRTLSDIS
NASSSFGWLSLDGDPTNFNEGSQVPERPPKPFPRRINSERKASSYQQGGG
ATANPVATAPSPQLSSEIERLMSQGYSYODIQKALVIAHNNIEMAKNILR
EFVSISSPAHVAT
Drosophila CBL-B mRNA sequence
(public gi: 1842452)

(SEQ ID NO: 151)

CATCTCGAAAATATTGTGTGGGTTTAAAAAACGTTAACGTCGCCGAAACG
CGTAGCCCCAAATGCACACGCCAGGTGCAAGGATAAAGCCGTGAGGATCG
GGCACCCAATCGGATAGATCGCGTTTGGTTAGCTTGTGGGGGAAAATCGT
ACTTAAGTCACCACTACTACTACACACGGGCACCACCAGCAACACCAACA
ACAACAACAACGAGAACAGCACCAGCAACAACAACAACAGCAGCAAGAAG
GAGAAGAGCTGAGAAGAGGAAGCAGAGGCAGCGCAGTCGGCAGCGCAGCG
GCAGAGAGAAAAGATGGCGACGAGAGGCAGTGGAACCCGTGTGCAATCGC
AGCCAAAGATTTTCCCATCGCTGCTTTCCAAGCTGCACGGCGCTATCTCG
GAAGCCTGCGTCTCGCAGCGTCTCTCCACCGACAAGAAGACGCTGGAGAA
GACCTGGAAGTTGATGGACAAGGTGGTCAAACTGTGCCAGCAGCCGAAGA
TGAATCTTAAGAATAGTCCACCGTTTATTTTGGACATCCTGCCGGATACG
TACCAGCGCCTGACATTGATCTACTCAAAGAAGGAGGACCAGATGCACCT
GCTCCATGCCAACGAGCACTTCAACGTGTTCATCAACAACCTGATGCGAA
AGTCCAAGCGGGCCATCAAGTTGTTCAAGGAGGGCAAGGAGAAGATGTTC
GACGAGAACTCCCACTACCGCCGCAATCTCACCAAGCTCAGCCTGGTCTT
CTCCCACATGCTCAGCGAACTGAAGGCCATATTCCCCAACGGTGTCTTTG
CCGGGGATCAATTTCGGATCACCAAAGCGGATGCGGCTGACTTTTGGAAG
AGCAACTTCGGTAACAGCACATTGGTTCCCTGGAAAATCTTCCGGCAGGA
GCTTAGCAAAGTACATCCCATAATCTCCGGCCTGGAGGCCATGGCCCTAA
AGACCACTATCGATCTTACCTGCAACGACTTCATTTCCAACTTCGAGTTC
GACGTCTTCACACGCCTCTTCCAGCCTTGGGTGACACTGCTACGCAACTG
GCAGATTCTGGCCGTCACACATCCGGGCTACGTGGCGTTTCTCACATACG
ACGAGGTGAAGGCTCGCCTACAGCGCTACATCCTCAAGGCGGGCAGCTAC
GTTTTCCGGCTCTCCTGCACGCGATTGGGCCAATGGGCCATCGGCTACGT
AACTGCCGAGGGAGAGATTCTGCAGACAATCCCTCAGAACAAGTCGCTGT
GCCAGGCGCTGCTCGATGGCCATCGAGAGGGCTTCTACTTGTACCCAGAT
GGCCAAGCGTACAATCCGGATCTGTCGTCTGCCGTTCAAAGTCCCACAGA
GGACCACATAACCGTTACCCAAGAGCAATACGAACTATACTGTGAAATGG
GCAGCACCTTTCAGCTGTGCAAAATTTGTGCGGAGAACGACAAAGATATC
CGCATCGAGCCCTGTGGCCACTTGTTGTGCACTCCCTGCCTTACCTCCTG
GCAAGTGGATTCCGAGGGACAGGGCTGCCCCTTCTGTCGGGCCGAAATCA
AGGGCACCGAACAAATCGTTGTGGACGCTTTCGATCCGCGCAAGCAACAC
AACCGGAACGTCACCAATGGGCGACAGCAGCAGCAGGAAGAAGACGACAC
TGAGGTATAGTTTTGTTCACAGCCTGATCAGCCTGATCCGCCTGCTCCGC
TGCCGCCTGTGCTGCTATTTATATACATATTACTCTTATGATTACCTTTG
GTTCGTTTATACAGTTATATATGCCTATATATACATTATATATTTTAGAT
TTTACAACTGCTATTGTTTATATAAGTTTAATGTTTAGCCTGCAGTTCGC
AGTGGCAGTTTCGAGTTTAATTTTGTTTGTTTAGCTGTAACATATTTAAA
TTATTAGCCAAACTCATGCAACTAACATCCACAGACCCACGCACACACGC
CCAATCACAAGCACAAGTACAACCATAACCATTGTCCATCCATCGAGCAC
ATGCATAACGTAGTTAAAGTTCTTTGACCGGAAGTCGCTCATCAACCATC
GTTTGCTATCGCTTCCTCTGTTTTCTCTCCGCCGGTTTGGTTTGGTTTGG
TTTGTGTGCGTTCGTTTAGTTGTTCGTTCTTCCACTCTCACGCTCTCTCT
ATCTATTGATCACGTTCGCCTCTGTTTATGAATCATATTTTAATCGATTC
GATTCGCCCTCGATTGCACTTTTGTACATAGGCACTATGGAATTTATAAT
TGGTAACCTTGTTCTTGTATTATTCGGGTGAATTTTCTCCTTTCACATCC
AGCTTGATTATCCCCTTGATTATGTATGCCCGCCAGTAATTTTTGTATCT
ATCCCCTACTCTAGAATCATTCTCTTAATCATTGTACTCCGTTATGTGTT
TATTTCATTTTAGTTTATTGTTTAATACTTCCAAAGATACATTTAGTTTG
TAGTAGCCTGCGTTTACTTCCCCCCCATATCAATTCAATTTTATTTGTAA
GCAGCCAAYGCGCTGCCCTAAGACTGTAATTTATTATTAACAMAAAAAAR
AAAATCGAAAAAGTTTAAGAAATCAGGCTAAACATAGGAGGCCTCGAATC
GATCGATAATTTAGTTAGATTGYATGTAAATTAATTATTGATTTCCTGTG
TCACAAGGCCA
Drosophila CBL-B Protein sequence
(public gi: 1842453)

(SEQ ID NO: 152)

MATRGSGTRVQSQPKIFPSLLSKLHGAISEACVSQRLSTDKKTLEKTWKL
MDKVVKLCQQPKNNLKNSPPFILDILPDTYQRLRLIYSKKEDQMMLLHAN
EHFNVFINNLMRKCKRAIKLFKEGKEKMFDENSHYRRNLTKLSLVFSHML
SELKAIFPNGVFAGDQFRITKADAADFWKSNFGNSTLVPWKIFRQELSKV
HPIISGLEAMALKTTIDLTCNDFISNFEFDVFTRLFQPWVTLLRNWQILA
VTHPGYVAFLTYDEVKARLQRYILKAGSYVFRLSCTRLGQWAIGYVTAEG
EILQTIPQNKSLCQALLDGHREGFYLYPDGQAYNPDLSSAVQSPTEDHIT
VTQEQYELYCEMGSTFQLCKICAENDKDIRIEPCGHLLCTPCLTSWQVDS
EGQGCPFCRAEIKGTEQIVVDAFDPRKQHNRNVTNGRQQQQEEDDTEV
C. elegans CBL-B mRNA sequence
(public gi: 25150544)

(SEQ ID NO: 153)

CTATGATCATTACATCCTAATTAATTGCCACTGGACTTCACATCATATCA
CCGTTTCACCGGGAATCGGTTCAATAAACACAATTTTTCACCGGATACAT
CGGTTTGTCAATGGCACAGGCAATAATGCGCGATTTGTTCCCAGCACAAA
CAACTCGACGGAAGCGTTGACACTCAGTCCGAGAGCTGTTCCCAGCACAG
TTTCACTATTCGAAATCCCATCAGCTTCGGAGATGCCCGGTTTCTGCAGT
GAAGAGGATCGTCGATTTTTGCTCAAAGCATGCAAGTTTATGGATCAAGT
AGTGAAGAGTTGTCATAGCCCAAGACTGAATTTGAAAAATTCGCCGCCTT
TCATTTTGGACATTCTACCTGATACTTATACGCATTTAATGCTGATATTC
ACACAAAACAATGACATACTCCAAGACAACGACTACTTGAAAATCTTTCT
GGAGAGTATCATCAACAAGTGCAAAGAGATCATCAAACTGTTCAAGACGT
CAGCTATCTACAATGACCAGTCTGAAGAACGACGGAAGCTTACGAAAATG
TCACTAACATTTTCACATATGCTTTTCGAGATTAAAGCATTATTTCCGGA
AGGTATCTATATTGAAGACCGGTTTCGGATGACAAAGAAGGAACCCGAAA
GCTTTTGGAGTCATCATTTTACAAAAAAAAACATTGTACCCTGGTCAACA
TTTTTTACTGCATTAGAAAAGCACCATGGATCAACGATAGGAAAAATGGA
AGCAGCCGAATTAAAAGCTACGATAGACTTGAGCGGAGATGATTTTATTT
CGAATTTTGAGTTTGATGTGTTTACAAGGTTATTCTACCCTTTCAAAACA
CTGATCAAAAATTGGCAAACACTCACCACCGCCCATCCCGGATACTGTGC
ATTTCTCACATACGATGACGTCAAAAAACGGTTAGAAAAATTAACGAAAA
AACCTGGAAGCTACATCTTCCGGTTATCATGCACACGTCCTGGACAATGG
GCAATAGGATACGTAGCTCCGGATGGAAAGATTTATCAGACAATACCACA
GATCAAGATATTAACTTATCCAAATTGATOGATGTGCCACAAGCGGACAG
AGTGCAAGTGACCAGTGAACAATACGAGTTGTATTGTGACATGGGCACAA
CATTCGAGTTGTGCAAAATTTGTGACGATAACGAGAAGAACATCAAAATT
GAGCCATGTGGACATTTGCTCTGCGCAAAATGTTTGGCTAACTGGCAGGA
TTCGGATGGTGGTGGCAACACATGTCCATTCTGCCGCTACGAAATCAAAG
GAACAAATCGTGTGATTATTGACAGGTTCAAGCCCACTCCGGTAGAAATT
GAAAAAGCGAAAAATGTAGCTGCTGCGGAGAAGAAGCTGATCTCATTAGT
TCCCGACGTGCCTCCCAGAACGTATGTGTCCCAATGTTCTCAAAGTTTGC
TGCATGACGCGTCAAACTCAATTCCGTCGGTCGACGAGTTGCCGTTGGTG
CCGCCACCGTTGCCACCGAAAGCATTGCCTACCCTGGACACTTTGAATTC
GTCACAAACATCCTCTTCATACGTGAACATCAAAGAGCTGGAAAATGTTG
AAACAAGCGGAGAAGCATTGGCACAAGTGGTAAACCGGCAACGGGCGCCT
TCAATCCAAGCTCCACCACTACCGCCAAGGTTATCAGCGAGCGAGCACCA
ACCACACCACCCATACACAAATACGAACAGTGAGCCGGAGTAGACTTGTG
TAAATGTTCATCTTACCGCTTTATACTGCAATTTTCATTCCCCCACTTAT
CATAGAACTATTCTTCCACAACAACATATTGCCGTGACTAGAACTGGTAA
CACTACATCATTCTTTGTTAAAACGTTATTATATCTCTATTTCTTTTTCG
CCTACTCCTTTCCGTTTTTTTTTCAAATTTTGTCAATTTTCCTACAGCGT
TCTGACTCCTATTGGTAAGCAATCATGTCATATCTTGTTAAATTTTCATG
TTAATTTCTTACTCTCGCTGTCCCAGATTTTACGGAGTTTTCAGGAAACG
TTTGATTTTGTTCTATTCTACAATTTCCATCGCCCCCAACCTGTCGTGTA
TTTTCTATGTGTCACTCTGAAGAAAACAAGTTTAGACTTTTTAAAAATCG
TTTTATTACTCTAAAACTTAAAAGCTGAAATGTCAGCTATAGTAAAAATA
CATA
C. elegans CBL-B Protein sequence
(public gi: 25150545)

(SEQ ID NO: 154)

MGSINTIFHRIHRFVNGTGNNARFVPSTNNSTEALTLSPRAVPSTVSLFE
IPSASEMPCFCSEEDRRFLLKACKFMDQVVKSCHSPRLNLKNSPPFILDI
LPDTYTHLMLIFTQNNDILQDNDYLKIFLESMINKCKEIIKLFKTSAIYN
DQSEERRKLTKMSLTFSHMLFEIKALFPEGIYIEDRFRMTKKFAESFWSM
HFTKKNIVPWSTFFTALEKHHGSTIGKMEAAELKATIDLSGDDFISNFEF
DVFTRLFYPFKTLIKNWQTLTTAHPGYCAFLTYDEVKKRLEKLTKKPGSY
IFRLSCTRPGQWAIGYVAPDGKIYQTIPQNKSLIQALHEGHKEGFYIYPN
GRDQDINLSKLMDVPQADRVQVTSEQYELYCEMGTTFELCKICDDNEKNI
KIEPCGHLLCAKCIANWQDSDGGGNTCPFCRYEIKGTNRVIIDRFKPTPV
EIEKAKNVAAAEKKLISLVPDVPPRTYVSQCSQSLLHDASNSIPSVDELP
LVPPPLPPKALGTLDTLNSSQTSSSYVNIKELENVETSGEALAQVVNRQR
APSIQAPPLPPRLSASEMQPHHPYTNTNSERE

POSH-associated proteins were identified by using a yeast two-hybrid assay. Procedure:

Bait plasmid (GAL4-BD) was transformed into yeast strain AH109 (Clontech) and transfromants were selected on defined media lacking tryptophan. Yeast strain Y187 containing pre-transformed Hela cDNA prey(GAL4-AD) library (Clontech) was mated according to the Clontech protocol with bait containing yeast and plated on defined media lacking tryptophan, leucine, histidine and containing 2 mM 3 amino triazol. Colonies that grew on the selective media were tested for beta-galactosidase activity and positive clones were further characterized. Prey clones were identified by amplifying cDNA insert and sequencing using vector derived primers.

Bait:

Plasmid vector: pGBK-T7 (Clontech)

Plasmid name: pPL269- pGBK-T7 GAL4 POSHdR

Protein sequence: Corresponds to aa 53-888 of POSH (RING domain deleted)

RTLVGSGVEELPSNILLVRLLDGIKQRPWKPGPGGGSGTNCTNALRSQSS
TVANCSSKDLQSSQGGQQPRVQSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI
Library screened: Hela pretransformed library
(Clontech).

One regulatory subunit (e.g., PRKAR1A) of PKA was identified as a POSH-AP by yeast two-hybrid screen. Since both a regulatory subunit and a catalytic subunit are required for the PKA function, it is likely that a catalytic subunit of PKA (e.g., PRKACA or PRKACB) may be a POSH-AP and form a complex with POSH.

Examples of sequences for a regulatory subunit of PKA (PRKAR1A) and two catalytic subunits of PKA (PRKACA and PRKACB) are presented below.

Human PRKAR1A mRNA sequence - var1
(public gi: 23273779)

(SEQ ID NO: 98)

GGTGGAGCTGTCGCCTAGCCGCTATCGCAGAGTCGAGCGGGGCTGGGAGC
AAAGCGCTGAGGGAGCTCGGTACGCCGCCGCCTCGCACCCGCAGCCTCGC
GCCCGCCGCCGCCCGTCCCCAGAGAACCATGGAGTCTGGCAGTACCGCCG
CCAGTGAGGAGGCACGCAGCCTTCGAGAATGTGAGCTCTACGTCCAGAAG
CATAACATTCAAGCGCTGCTCAAAGATTCTATTGTGCAGTTGTGCACTGC
TCGACCTGAGAGACCCATGGCATTCCTCAGGGAATACTTTGAGAGGTTCG
AGAAGGAGGAGGCAAAACAGATTCAGAATCTGCAGAAAGCAGGCACTCGT
ACAGACTCAAGGGAGGATGAGATTTCTCCTCCTCCACCCAACCCAGTGGT
TAAAGGTAGGAGGCGACGAGGTGCTATCAGCGCTGAGGTCTACACGGAGG
AAGATGCGGCATCCTATGTTAGAAAGGTTATACCAAAAGATTACAAGACA
ATGGCCGCTTTAGCCAAAGCCATTGAAAAGAATGTGCTGTTTTCACATCT
TGATGATAATGAGAGAAGTGATATTTTTGATGCCATGTTTTCGGTCTCCT
TTATCGCAGGAGAGACTGTGATTCAGCAAGGTGATGAAGGGGATAACTTC
TATGTGATTGATCAAGGAGAGACGGATGTCTATGTTAACAATGAATGGGC
AACCAGTGTTGGGGAAGGAGGGAGCTTTGGAGAACTTGCTTTGATTTATG
GAACACCGAGAGCAGCCACTGTCAAAGCAAAGACAAATGTGAAATTGTGG
GGCATCGACCGAGACAGCTATAGAAGAATCCTCATGGGAAGCACACTGAG
AAAGCGGAAGATGTATGAGGAATTCCTTAGTAAAGTCTCTATTTTAGAGT
CTCTGGACAAGTGGGAACGTCTTACGGTAGCTGATGCATTGGAACCAGTG
CAGTTTGAAGATCGGCAGAAGATTGTGGTGCAGGGAGAACCAGGGGATGA
GTTCTTCATTATTTTAGAGGGGTCAGCTGCTGTGCTACAACGTCGGTCAG
AAAATGAAGAGTTTGTTGAAGTGGGAAGATTGGCGCCTTCTGATTATTTT
GGTGAAATTGCACTACTGATGAATCGTCCTCGTGCTGCCACAGTTGTTGC
TCGTGGCCCCTTGAAGTGCGTTAAGCTGGACCGACCTAGATTTGAACGTG
TTCTTGGCCCATGCTCAGACATCCTCAAACGAAACATCCAGCAGTACAAC
AGTTTTGTGTCACTGTCTGTCTGAAATCTGCCTCCTGTGCCTCCCTTTTC
TCCTCTCCCCAATCCATGCTTCACTCATGCAAACTGCTTTATTTTCCCTA
CTTGCAGCGCCAAGTGGCCACTGGCATCGCAGCTTCCTGTCTGTTTATAT
ATTGAAAGTTGCTTTTATTGCACCATTTTCAATTTGGAGCATTAACTAAA
TGCTCATACACAGTTAAATAAATAGAAAGAGTTCTATGGAGACTTTGCTG
TTACTGCTTCTCTTTGTGCAGTGTTAGTATTCACCCTGGGCAGTGAGTGC
CATGCTTTTTGGTGACGGCAGATCCCAGCACCTATTGAATTACCATAGAG
TAATGATGTAACAGTGCAAGATTTTTTTTTTAAGTGACATAATTGTCCAG
TTATAAGCGTATTTAGACTGTGGCCATATATGCTGTATTTCTTTGTAGAA
TAAATGGTTTCTCATTAAACTCTAAAGATTAGGGAAAATGGATATAGAAA
ATCTTAGTATAGTAGAAAGACATCTCCCTGTAATTAAACTAGTTTAAGGG
TGGAAAAATGCCCATTTTTGCTAATTATCAATGGGATATGATTGGTTCAG
TTTTTTTTTTTCCAGAGTTGTTGTTTGCCAAGCTAATCTGCCTGGTTTTA
TTTATATCTTGTTATTAATGTTTCTTCTCCAATTCTGAAATACTTTTGAG
TATGGCTATCTATACCTGCCTTTTAAGTTTGAAACTAACTCATAGATTGC
AAATATTGGTTAGTATTTAACTACATCTGCCTCGGCTCACAAATTCCGAT
TAGACCTTTATCCAGCTAGTGCCAAATAATTGATCAGATGCTGAATTGAG
AATAAGAATTGAGGTCTACATTCTTGGTTGTTAATTTAGAGCGTTTGGTT
AAAGTATGTCCTTTCAGCTGACTCCAGTATAATCTCCTCTGCTCATTAAA
CTGATTCCAGGAGATTCGATTTGCTGTGACTAGATACAGATGGAGCAAAT
GTCCTAACAGAGAAATAGAGGTGATGCTGCTAAAGGGAGAAATGCCAGGC
GGACAAAGTTCAGTGTCGGGAATTTTCCCCGTGACATTCACTGGGGCATG
AGATTTTGGAAGAAGTTTTTTACTTTCGTTTAGTCTTTTTTTCCTTCCTT
TTTATTCAGCTAGAATTTCTGGTGGGTTGATGGTAGGGTATAATGTGTCT
GTGTTGCTTCAAATTGGTCTGAAAGGCTATCCTGCGGAAAGTCCTGCTTT
CCTATCTAGCATTTATTTCTCTCGCAAACTTTTCTTTCTTTTCTTTTTTA
AAGTAAACTTGTGTATTGAGTCTTAACTGTATTTCAGTATTTTCCAGCCT
TATGTGTTACATTATTCCAATGATACCCAACAGTTTATTTTTATTATTTT
TTTAAACAAAATTTCACAGTTCTGTAATGTAGGCACTTTTATTTTCATTG
TGATTTATATATAAGGTAATCTAGGGTTATATTTGGGAGTGACTGCAAGC
ATTTTTCCATCTGTGTGCAACTAACTGACTCTGTTATTGATCCCTTCTCC
TGCCCTTTCCCAGGTAATTTAAATTGGTCATGGTAGATTTTTTTCATAGA
TTTGAAAAACTTTTAGGTTGTTACCAAGTATGAAGTATAAATCTGGGGAA
GAGGTTTTATTTACATTTTAGGGTGGGTAAGAAAGCCACCTTGTTACAAA
TTTTTTAATTTCCAAAATAATCTATATTAAATGAGGGTTTCTGATCTGTA
CTTTGTGTTTAGCTACCTTTTTATATTTAAAAAATTAAAAATGAAAATTA
CGTTCTTACAAGCTTAAAGCTTGATTTGATCTTTGTTTAAATGCCAAAAT
GTACTTAAATGAGTTACTTAGAATGCCATAAAATTGCAGTTTCATGTATG
TATATAATCATGCTCATGTATATTTAGTTACGTATAATGCTTTCTGAGTG
AGTTTTACTCTTAAATCATTTGGTTAAATCATTTGGCTTGCTGTTTACTC
CCTTCTGTAGTTTTTAATTAAAAACTTTAAAGATAAGTCTACATTAAACA
ATGATCACATCTAAAGCTTTATCTTTGTGTAATCTAAGTATATGTGAGAA
ATCAGAATTGGCATAATTTGTCTTAGTTGATATTCAAGGCTTTAAAAGTC
ATTATTCCTGGGCTTGGTAAGTGAATTTATGAGATTTACTGCTCTAGAAA
GTATAGATGGCGAAAGGACCGTTTTGTATTGCTTCCTGATTACCAGTCTG
ATTATACCATGTGTGCTAATATACTTTTTTTGTTATAGATTGTCTTAATG
GTAGGTCAAGTAATAAAAAGAGATGAAATAATTTAAAAAAAAAAAAAAAA
Human PRKAR1A mRNA sequence - var2
(public gi: 1658305)

(SEQ ID NO: 99)

AGAGGCGTCAAGGGAGGCCGGAGGGAGAGTGGGGTGGACAGAGGAGCGGA
GGGACGAGAGGGAAGCGCACGATAGCTGCGCGGAGAGAGAGCGAAGAGCA
GGAGGAGGAACAAAGGCGACCCAAGACACCCAGAGAGGGACAGAGAACCA
TGGAGTCTGGCAGTACCGCCGCCAGTGAGGAGGCACGCAGCCTTCGAGAA
TGTGACCTCTACGTCCAGAAGCATAACATTCAAGCGCTGCTCAAAGATTC
TATTGTGCAGTTGTGCACTGCTCGACCTGAGAGACCCATGGCATTCCTCA
GGGAATACTTTGAGAGGTTGGAGAAGGAGGAGGCAAAACAGATTCAGAAT
CTCCAGAAAGCAGGCACTCGTACAGACTCAAGGGAGGATGAGATTTCTCC
TCCTCCACCCAA
Human PRKAR1A mRNA sequence - var3
(public gi: 21757396)

(SEQ ID NO: 100)

TAATTTTCTTGTGTGTTTTTAAAAATTTTGATTATGCTAGTAGTTGGCTA
ATCAGATCCTCACTCCAGTGGTTTGCTCTGTGACGTTAGGATACTCCCAT
GGGATAGAAGTTACGTATAGGGAATGTCAGATATTCTTCATTGTGCTGAC
TTGCTTTCGCTTACAGTTGACTTTTGTGCCCTGGTAATTCTGTATCCTGT
TTACCGTTTACCTACTTCCCACGTCATCATGATTTCTTTTGAGGGAGAAC
TGAATGAAATTCCCTTAAGGGCCTGACTTCAGCACCCGTCTCTGCAGAGG
TTAGTGGCTCATACTTCCTCCCAGGAGCTGAGGTTATCGACTCTCACTGT
TGCCTACAGAGCACAGATCCTGAACTAAATGAAACATTTACTTGGAATAA
TGCTAATTCTGTACATATTTTATTCCCTAGTCCCCACTTCCCTGTTTAAA
AACAAAATCTACTTAGAAAAAAATCCCTGTGAATCAGTTGTCTAATGAAT
TTAGCAAGTTAAATGCCAGATTGACATTTTGCTTTATAGTTTATACAAGC
ATGTGTGTGTTTTTTTCTCGCAGAGAACCATGGAGTCTGGCAGTACCGCC
GCCAGTGAGGAGGCACGCAGCCTTCGAGAATGTGAGCTCTACGTCCAGAA
GCATAACATTCAAGCGCTGCTCAAAGATTCTATTCTGCAGTTGTGCACTG
CTCGACCTGAGAGACCCATGGCATTCCTCAGGGAATACTTTGAGAGGTTG
GAGAAGGAGGAGGCAAAACAGATTCAGAATCTGCAGAAAGCAGGCACTCG
TACAGACTCAAGGGAGGATGAGATTTCTCCTCCTCCACCCAACCCAGTGG
TTAAAGGTAGGAGGCGACGAGGTGCTATCAGCGCTGAGGTCTACACGGAG
GAAGATGCGGCATCCTATGTTAGAAAGGTTATACCAAAAGATTACAAGAC
AATGGCCGCTTTAGCCAAAGCCATTGAAAAGAATGTGCTGTTTTCACATC
TTGATGATAATGAGAGAAGTGATATTTTTGATGCCATGTTTTCGGTCTCC
TTTATCGCAGGAGAGACTGTGATTCAGCAAGGTGATGAAGGGGATAACTT
CTATGTGATTGATCAAGGAGAGACGGATGTCTATGTTAACAATGAATGGG
CAACCAGTGTTGGGGAAGGAGGGAGCTTTGGAGAACTTGCTTTGATTTAT
GGAACACCGAGAGCAGCCACTGTCAAAGCAAAGACAAATGTGAAATTGTG
CGGCATCGACCGAGACAGCTATAGAAGAATCCTCATCGGAAGCACACTGA
GAAAGCGGAAGATGTATGAGGAATTCCTTAGTAAAGTCTCTATTTTAGAG
TCTCTCGACAAGTGOGAACGTCTTACGGTAGCTGATGCATTGGAACCAGT
GCAGTTTGAAGATGGGCAGAAGATTGTGGTGCAGGGAGAACCAGGGGATG
AGTTCTTCATTATTTTAGAGGGGTCAGCTGCTGTGCTACAACGTCGGTCA
GAAAATGAAGAGTTTGTTGAAGTGGGAAGATTGGGGCCTTCTGATTATTT
TGGTGAAATTGCACTACTGATGAATCGTCCTCGTGCTGCCACAGTTGTTG
CTCGTGGCCCCTTGAAGTGCGTTAAGCTGGACCGACCTAGATTTGAACGT
GTTCTTGGCCCATGCTCAGACATCCTCAAACGAAACATCCAGCAGTACAA
CAGTTTTGTGTCACTGTCTGTCTGAAATCCGCCTCCTGTGCCTCCCTTTT
CTCCTCTCCCCAATCCATGCTTCACTCATGCAAACTGCTTTATTTTCCCT
ACTTGCAGCGCCAAGTGGCCACTGGCATCGCAGCTTCCTGTCTGTTTATA
TATTGAAAGTTGCTTTTATTGCACCATTTTCAATTTGGAGCATTAACTAA
ATGCTCATACACAGTTAAATAAATAGAAAGAGTTCTATGG
Human PRKAR1A mRNA sequence - var4
(public gi: 1526988)

(SEQ ID NO: 101)

GGCAGAGTGGAGCGGGGCTGGGAGCAAAGCGCTGAGGGAGCTCGGTACGC
CGCCGCCTCGCACCCGCAGCCTCGCGCCCGCCGCCGCCCGTCCCCAGAGA
ACCATGGAGTCTGGCACTACCGCCGCCAGTGAGGAGGCACGCAGCCTTCG
AGAATGTGAGCTCTACGTCCAGAAGCATAACATTCAAGCGCTGCTCAAAG
ATTCTATTGTGCAGTTGTGCACTGCTCGACCTGAGACACCCATGGCATTC
CTCACGGAATACTTTGAGAGGTTGGAGAAGGAGGAGGCAAAACAGATTCA
GAATCTGCAGAAAGCAGGCACTCGTACAGACTCAAGGGAGGATGAGATTT
CTCCTCCTCCACCCAACCCAGTGGTTAAAGGTAGGAGGCGACGAGGTGCT
ATCAGCGCTGAGGTCTACACGGAGGAAGATGCGGCATCCTATGTTAGAAA
GGTTATACCAAAAGATTACAAGACAATGGCCGCTTTAGCCAAAGCCATTG
AAAAGAATGTGCTGTTTTCACATCTTGATGATAATGAGAGAAGTGATATT
TTTGATGCCATGTTTTCGGTCTCCTTTATCGCAGGAGAGACTGTGATTCA
GCAAGGTGATGAAGGGGATAACTTCTATGTGATTGATCAAGGAGAGACGG
ATGTCTATGTTAACAATGAATGGGCAACCAGTGTTGGGGAAGGAGGGAGC
TTTGGAGAACTTGCTTTGATTTATGGAACACCGAGAGCAGCCACTGTCAA
AGCAAAGACAAATGTGAAATTGTGGGGCATCGACCGAGACAGCTATAGAA
GAATCCTCATGGGAAGCACACTGAGAAAGCGGAAGATGTATGAGGAATTC
CTTAGTAAAGTCTCTATTTTAGAGTCTCTGGACAAGTGGGAACGTCTTAC
GGTAGCTGATGCATTGGAACCAGTGCAGTTTGAAGATGGGCAGAAGATTG
TGGTGCAGGGAGAACCAGGGGATGAGTTCTTCATTATTTTAGAGGGGTCA
GCTGCTGTGCTACAACGTCGGTCAGAAAATGAAGAGTTTGTTGAAGTGGG
AAGATTGGGGCCTTCTGATTATTTTGGTGAAATTGCACTACTGATGAATC
GTCCTCGTGCTGCCACAGTTGTTGCTCGTGGCCCCTTGAAGTGCGTTAAG
CTGGACCGACCTAGATTTGAACGTGTTCTTGGCCCATGCTCAGACATCCT
CAAACGAAACATCCAGCAGTACAACAGTTTTGTGTCACTGTCTGTCTGAA
ATCTGCCTCCTGTGCCTCCCTTTTCTCCTCTCCCCAATCCATGCTTCACT
CATGCAAACTGCTTTATTTTCCCTACTTGCAGCGCCAAGTGGCCACTGGC
ATCGCAGCTTCCTGTCTGTTTATATATTAAAGTTGCTTTTATTGCACCAT
TTTCAATTTGGAGCATTAACTAAATGCTCATACACAGTTAAATAAATAGA
AAGAGTTCTATGGAAAAAAAAAAAAA
Human PRKAR1A mRNA sequence - var5
(public gi: 1526989)

(SEQ ID NO: 102)

GCTGGGAGCAAAGCCCTGAGGGAGCTCGGTACGCCGCCGCCTCGCACCCG
CAGCCTCGCGCCCCCCGCCGCCCGTCCCCAGAGAACCATGGAGTCTGGCA
GTACCGCCGCCAGTGAGGAGGCACGCAGCCTTCGAGAATGTGAGCTCTAC
GTCCAGAAGCATAACATTCAAGCGCTGCTCAAAGATTCTATTGTGCAGTT
GTGCACTGCTCGACCTGAGAGACCCATGGCATTCCTCAGGGAATACTTTG
AGAGGTTCGAGAAGGAGGAGGCAAAACAGATTCAGAATCTGCAGAAAGCA
GGCACTCGTACAGACTCAAGGGAGGATGAGATTTCTCCTCCTCCACCCAA
CCCAGTGGTTAAAGGTAGGAGGCGACGAGGTGCTATCAGCGCTGAGGTCT
ACACGGAGGAAGATGCGGCATCCTATGTTAGAAAGGTTATACCAAAAGAT
TACAAGACAATGGCCGCTTTAGCCAAAGCCATTGAAAAGAATGTGCTGTT
TTCACATCTTGATGATAATGAGAGAAGTGATATTTTTGATGCCATGTTTT
CGGTCTCCTTTATCGCAGGAGAGACTGTGATTCAGCAAGGTGATGAAGGG
GATAACTTCTATGTGATTGATCAAGGAGAGACGGATGTCTATGTTAACAA
TGAATGGGCAACCAGTGTTGGGGAAGGAGGGAGCTTTGGAGAACTTGCTT
TGATTTATGGAACACCGAGAGCAGCCACTGTCAAAGCAAAGACAAATGTG
AAATTGTGGGGCATCGACCGAGACAGCTATAGAAGAATCCTCATGGGAAG
CACACTGAGAAAGCGGAAGATGTATGAGGAATTCCTTAGTAAAGTCTCTA
TTTTAGAGTCTCTGGACAAGTGGGAACGTCTTACGGTAGCTGATGCATTG
GAACCAGTGCAGTTTGAAGATGGGCAGAAGATTGTCGTGCAGGGAGAACC
AGGGGATGAGTTCTTCATTATTTTAGAGGGGTCAGCTGCTGTGCTACAAC
GTCGGTCAGAAAATGAAGAGTTTGTTGAAGTGGGAAGATTGGGGCCTTCT
GATTATTTTGGTGAAATTGCACTACTGATGAATCGTCCTCGTGCTGCCAC
AGTTGTTGCTCGTGGCCCCTTGAAGTGCGTTAAGCTGGACCGACCTAGAT
TTGAACGTGTTCTTGGCCCATGCTCAGACATCCTCAAACGAAACATCCAG
CAGTACAACAGTTTTGTGTCACTGTCTGTCTGAAATCTGCCTCCTGTGCC
TCCCTTTTCTCCTCTCCCCAATCCATGCTTCACTCATGCAAACTGCTTTA
TTTTCCCTACTTGCAGCGCCAAGTGGCCACTGGCATCGCAGCTTCCTGTC
TGTTTATATATTGAAAGTTGCTTTTATTGCACCATTTTCAATTTGGAGCA
TTAACTAAATGCTCATACACAGTTAAATAAATAGAAAGAGTTCTATGGAG
ACTTTGCTGTTACTGCTTCTCTTTGTGCAGTGTTAGTATTCACCCTGGGC
AGTGAGTGCCATGCTTTTTGGTGAGGGCAGATCCAGCACCTATTGAATTA
CCATAGAGTAATGATGTAACAGTGCAAGATTTTTTTTTTTAAGTGACATA
ATTGTCCAGTTATAAGCGTATTTAGACTGTGGCCATATATGCTGTATTTC
TTTGTAGAATAAATGGTTTCTCATTAAACTCTAAAGATTAGGGAAATGGA
TATAGAAAATCTTAGTATAGTAGAAAGACATCTGCCTGTAATTAAACTAG
TTTAAGGGTGGAAAAATGAAAATTTTTGCTAATTATCAATGGGATATGAT
TGGTTCAGTTTTTTTTTTCCAGAGTTGTTGTTTGCCAAGCTAATCTGCCT
GGTTTATTTATATCTTGTTATTAATGTTTCTTCTCCAATTCTGAAATACT
TTTGAGTATGGCTATCTATACCTGCCTTTTAAGTTTGAAACTAACTCATA
GATGCAAATATTGGTTAGTATTTAACTACATCTGCCTCGGCTCACAAATT
CCGATTAGACCTTTATCCAGCTAGTGCCAAATAATTGATCAGATGCTGAA
TTGAGAATAAGAATTTGAGGTCTACATTCTTGGTTGTTAATTTAGAGCGT
TTGGTTAAAGTATGTCCTTCAGCTGACTCCAGTATAATCTCCTCTGCTCA
TTAAACTGATTCCAGGAGATTGGATTTGCTGTGACTAGATACAGATGGAG
CAAATGTCCTAACAGAGAAATAGAGGTGATGCTGCTAAAGGGAGAAATGC
CAGGCGGACAAAGTTCAGTGTCGGGAATTTTCCCCGTGACATTCACTGGG
GCATGAGATTTTGGAAGAAGTTTTTTACTTTGGTTTAGTCTTTTTTTCCT
CCTTTTTATTCAGCTAGAATTTCTGGTGGGTTGATGGTAGGGTATAATGT
GTCTGTGTTGCTTCAAATTGGTCTGAAAGGCTATCCTGCTGAAAGTCCTG
CTTTCCTATCTAGCATTTATTCCTCTCGCAAACTTTTCTTTCTTTTCTTT
TTTAAAGTAAACTTGTGTATTGAGTCTTAACTGTATTTCAGTATTTTCCA
GCCTTATGTGTTACATTATTCCAATGATACCCAACAGTTTATTTTTATTA
TTTTTTTAAACAAAATTTCACAGTTCTGTAATGTAGGCACTTTTATTTTC
ATTGTGATTTATATATAAGGTAATGTAGGGTTATATTTGGGAGTGACTGC
AAGCATTTTTCCATCTGTGTGCAACTAACTGACTCTGTTATTGATCCCTT
CTCCTGCCCTTTCCCAGGTAATTTAAATTGGTCATGGTAGATTTTTTTCA
TAGATTTGAAAAACTTTTAGGTTGTTACCAAGTATGAAGTATAAATCTGG
GGAAGAGGTTTTATTTACATTTTAGGGTGGGTAAGAAAGCCACCTTGTTA
CAAATTTTTTAATTTCCAAAATAATCTATATTAAATGAGGGTTTCTGATC
TGTACTTTGTGTTTAGCTACCTTTTTATATTTAAAAAATTAAAAATGAAA
ATTATGTTCTTACAAGCTTAAAGCTTGATTTGATCT
Human PRKAR1A mRNA sequence - var6
(public gi: 4506062)

(SEQ ID NO: 103)

GCTGGGAGCAAAGCCCTGAGGGAGCTCGGTACGCCGCCGCCTCGCACCCG
CAGCCTCGCGCCCGCCGCCGCCCGTCCCCAGAGAACCATGGAGTCTGGCA
GTACCGCCGCCAGTGACGAGGCACGCAGCCTTCGAGAATGTGAGCTCTAC
GTCCAGAAGCATAACATTCAAGCGCTGCTCAAAGATTCTATTGTGCAGTT
GTGCACTGCTCGACCTGACAGACCCATGGCATTCCTCAGGGAATACTTTG
AGAGGTTGGAGAAGGACGACGCAAAACAGATTCAGAATCTGCAGAAAGCA
GGCACTCGTACAGACTCAAGGGAGGATGAGATTTCTCCTCCTCCACCCAA
CCCAGTGGTTAAAGCTAGGAGGCGACGAGGTGCTATCAGCGCTGAGGTCT
ACACGGAGGAAGATGCGGCATCCTATGTTACAAAGGTTATACCAAAAGAT
TACAAGACAATGGCCGCTTTAGCCAAAGCCATTGAAAACAATGTGCTGTT
TTCACATCTTGATGATAATGAGAGAAGTGATATTTTTGATGCCATGTTTT
CGGTCTCCTTTATCGCAGGAGAGACTGTGATTCAGCAAGGTGATGAAGGG
GATAACTTCTATGTGATTGATCAAGGAGAGACGGATGTCTATGTTAACAA
TGAATGGGCAACCAGTGTTGGGGAAGGAGGGAGCTTTGGAGAACTTGCTT
TGATTTATGGAACACCGAGAGCAGCCACTGTCAAAGCAAAGACAAATGTG
AAATTGTGGGGCATCGACCGAGACAGCTATAGAAGAATCCTCATGGGAAG
CACACTGAGAAAGCGGAAGATGTATGAGGAATTCCTTAGTAAAGTCTCTA
TTTTAGAGTCTCTGGACAAGTGGGAACGTCTTACGGTAGCTGATGCATTG
GAACCAGTGCACTTTGAACATGGGCAGAAGATTGTGGTGCAGGGAGAACC
AGGGGATGAGTTCTTCATTATTTTAGAGGGGTCAGCTGCTGTGCTACAAC
GTCGGTCAGAAAATGAAGAGTTTGTTGAAGTGGGAAGATTGGGGCCTTCT
GATTATTTTGGTGAAATTGCACTACTGATGAATCGTCCTCGTGCTGCCAC
AGTTGTTGCTCGTGGCCCCTTGAAGTGCGTTAAGCTGGACCGACCTAGAT
TTGAACGTGTTCTTGGCCCATGCTCAGACATCCTCAAACGAAACATCCAG
CACTACAACAGTTTTGTGTCACTGTCTGTCTGAAATCTGCCTCCTGTGCC
TCCCTTTTCTCCTCTCCCCAATCCATGCTTCACTCATCCAAACTGCTTTA
TTTTCCCTACTTGCAGCGCCAAGTGCCCACTGGCATCGCAGCTTCCTGTC
TGTTTATATATTCAAAGTTCCTTTTATTGCACCATTTTCAATTTGGAGCA
TTAACTAAATGCTCATACACAGTTAAATAAATAGAAAGAGTTCTATCGAG
ACTTTGCTGTTACTGCTTCTCTTTGTGCAGTGTTAGTATTCACCCTGGGC
AGTGAGTGCCATGCTTTTTCGTGAGGGCAGATCCAGCACCTATTGAATTA
CCATAGAGTAATGATGTAACAGTGCAAGATTTTTTTTTTTAACTGACATA
ATTGTCCAGTTATAAGCGTATTTAGACTGTGGCCATATATGCTGTATTTC
TTTGTAGAATAAATGGTTTCTCATTAAACTCTAAAGATTAGGGAAATGGA
TATAGAAAATCTTAGTATAGTAGAAAGACATCTGCCTGTAATTAAACTAG
TTTAAGGGTGGAAAAATGAAAATTTTTGCTAATTATCAATGGGATATGAT
TGGTTCAGTTTTTTTTTTCCAGAGTTGTTGTTTGCCAAGCTAATCTGCCT
GGTTTATTTATATCTTGTTATTAATGTTTCTTCTCCAATTCTGAAATACT
TTTGAGTATGGCTATCTATACCTGCCTTTTAAGTTTGAAACTAACTCATA
GATGCAAATATTGGTTAGTATTTAACTACATCTGCCTCGGCTCACAAATT
CCGATTAGACCTTTATCCAGCTAGTGCCAAATAATTGATCAGATGCTGAA
TTGAGAATAAGAATTTGACGTCTACATTCTTGGTTGTTAATTTAGAGCGT
TTGGTTAAAGTATGTCCTTCAGCTGACTCCAGTATAATCTCCTCTGCTCA
TTAAACTGATTCCAGGAGATTGGATTTGCTGTGACTAGATACAGATGGAG
CAAATGTCCTAACAGAGAAATAGAGGTCATGCTGCTAAAGGGAGAAATGC
CAGGCGGACAAAGTTCAGTGTCCGGAATTTTCCCCGTGACATTCACTGGG
GCATGAGATTTTGGAAGAAGTTTTTTACTTTGGTTTAGTCTTTTTTTCCT
CCTTTTTATTCAGCTAGAATTTCTGGTGGGTTGATGGTAGGGTATAATGT
GTCTGTGTTGCTTCAAATTGGTCTGAAAGGCTATCCTGCTGAAAGTCCTG
CTTTCCTATCTAGCATTTATTCCTCTGGCAAACTTTTCTTTCTTTTCTTT
TTTAAAGTAAACTTGTGTATTGAGTCTTAACTGTATTTCAGTATTTTCCA
GCCTTATGTGTTACATTATTCCAATGATACCCAACAGTTTATTTTTATTA
TTTTTTTAAACAAAATTTCACAGTTCTGTAATGTACGCACTTTTATTTTC
ATTGTGATTTATATATAAGGTAATGTAGGGTTATATTTGGGAGTGACTGC
AAGCATTTTTCCATCTGTGTGCAACTAACTGACTCTGTTATTGATCCCTT
CTCCTGCCCTTTCCCAGGTAATTTAAATTGGTCATGGTAGATTTTTTTCA
TAGATTTGAAAAACTTTTAGGTTGTTACCAAGTATGAAGTATAAATCTCG
GGAAGAGGTTTTATTTACATTTTAGGGTGGGTAAGAAAGCCACCTTGTTA
CAAATTTTTTAATTTCCAAAATAATCTATATTAAATGACGGTTTCTGATC
TGTACTTTGTGTTTAGCTACCTTTTTATATTTAAAAAATTAAAAATGAAA
ATTATGTTCTTACAAGCTTAAAGCTTGATTTGATCT
Human PRKAR1A mRNA sequence - var7
(public gi: 4884279)

(SEQ ID NO: 104)

TATTTTCCAGCCTTATGTGTTACATTATTCCAATGATACCCAACACTTTA
TTTTTATTATTTTTTTAAACAAAATTTCACACTTCTGTAATGTAGGCACT
TTTATTTTCATTGTGATTTATATATAAGGTAATGTAGGGTTATATTTGGG
ACTGACTGCAAGCATTTTTCCATCTGTGTGCAACTAACTGACTCTGTTAT
TGATCCCTTCTCCTGCCCTTTCCCAGGTAATTTAAATTGGTCATGGTAGA
TTTTTTTCATAGATTTGAAAAACTTTTAGGTTCTTACCAAGTATGAAGTA
TAAATCTGGGGAAGAGGTTTTATTTACATTTTAGGGTGGGTAAGAAAGCC
ACCTTGTTACAAATTTTTTAATTTCCAAAATAATCTATATTAAATGACGG
TTTCTGATCTGTACTTTGTGTTTAGCTACCTTTTTATATTTAAAAAATTA
AAAATGAAAATTACGTTCTTACAAGCTTAAAGCTTGATTTGATCTTTGTT
TAAATGCCAAAATGTACTTAAATGAGTTACTTAGAATGCCATAAAATTGC
AGTTTCATGTATGTATATAATCATGCTCATGTATATTTAGTTACGTATAA
TGCTTTCTGAGTGAGTTTTACTCTTAAATCATTTGGTTAAATCATTTGGC
TTGCTGTTTACTCCCTTCTGTAGTTTTTAATTAAAAACTTTAAAGATAAG
TCTACATTAAACAATGATCACATCTAAAGCTTTATCTTTGTCTAATCTAA
GTATATGTGAGAAATCAGAATTGGCATAATTTGTCTTAGTTGATATTCAA
GGCTTTAAAAGTCATTATTCCTGGGCTTGGTAAGTGAATTTATGAGATTT
ACTCCTCTAGAAACTATAGATGGCCAAAGGACCGTTATGTATTGCTTCCT
GATTACCAGTCTGATTATACCATCTGTGCTAATATACTTTTTTTGTTATA
GATTGTCTTAATGGTAGGTCAAGTAATAAAAAGAGATGAATAATTTAAAA
AAAAAAAAAA
Human PRKAR1A Protein sequence - var1
(public gi: 1658306)

(SEQ ID NO: 116)

MESGSTAASEEARSLRECELYVQKHNIQALLKDSIVQLCTARPERPMAFL
REYFERLEKEEAKQIQNLQKAGTRTDSREDEISPPPP
Human PRKAR1A Protein sequence - var2
(public gi: 23273780)

(SEQ ID NO: 117)

MESGSTAASEEARSLRECELYVQKHNIQALLKDSIVQLCTARPERPMAFL
REYFERLEKEEAKQIQNLQKAGTRTDSREDEISPPPPNPVVKGRRRRGAI
SAEVYTEEDAASYVRKVIPKDYKTMAALAKAIEKNVLFSHLDDNERSDIF
DANFSVSFIAGETVIQQGDEGDNFYVIDQGETDVYVNNEWATSVGEGGSF
GELALIYGTPRAATVKAKTNVKLWGIDRDSYRRILMGSTLRKRKMYEEFL
SKVSILESLDKWERLTVADALEPVQFEDGQKIVVQGEPGDEFFIILEGSA
AVLQRRSENEEFVEVGRLGPSDYFGEIALLNNRPRAATVVARGPLKCVKL
DRPRFERVLGPCSDILKRNIQQYNSFVSLSV
Human PRKACA mRNA sequence - var1
(public gi: 24980835)

(SEQ ID NO: 105)

TCGGGCTGAGGTTCCCGGGCGGGCGGGCGCGGAGAGACGCGGGAAGCAGG
GGCTGGGCGGGGGTCGCGGCGCCGCAGCTAGCGCAGCCAGCCCGAGGGCC
GCCGCCGCCGCCGCCCAGCGCGCTCCGGGGCCGCCGGCCGCAGCCAGCAC
CCGCCGCGCCGCAGCTCCGGGACCGGCCCCGGCCGCCGCCGCCGCGATGG
GCAACGCCGCCGCCGCCAAGAAGGGCAGCGAGCAGGAGAGCGTGAAAGAA
TTCTTAGCCAAAGCCAAAGAAGATTTTCTTAAAAAATGGGAAAGTCCCGC
TCAGAACACAGCCCACTTGGATCAGTTTGAACGAATCAAGACCCTCGGCA
CGGGCTCCTTCGGGCGGGTGATGCTGGTGAAACACAAGGAGACCGGGAAC
CACTATGCCATGAAGATCCTCGACAAACAGAAGGTGGTGAAACTGAAACA
GATCGAACACACCCTGAATGAAAAGCGCATCCTGCAAGCTGTCAACTTTC
CGTTCCTCGTCAAACTCGAGTTCTCCTTCAACGACAACTCAAACTTATAC
ATGGTCATCGAGTACGTGCCCGGCGGGGAGATGTTCTCACACCTACGGCG
GATCGGAAGGTTCAGTGAGCCCCATGCCCGTTTCTACGCGGCCCAGATCG
TCCTGACCTTTGAGTATCTGCACTCGCTGGATCTCATCTACAGGGACCTG
AAGCCGGAGAATCTGCTCATTGACCAGCAGGGCTACATTCAGGTGACAGA
CTTCGGTTTCGCCAAGCGCGTGAAGGGCCGCACTTGGACCTTGTGCGGCA
CCCCTGAGTACCTCGCCCCTGAGATTATCCTGAGCAAAGGCTACAACAAG
GCCGTGGACTGGTGGGCCCTGGGGGTTCTTATCTATGAAATCGCCGCTGG
CTACCCGCCCTTCTTCGCAGACCAGCCCATCCAGATCTATGAGAAGATCG
TCTCTGGGAAGGTGCGCTTCCCTTCCCACTTCAGCTCTGACTTGAAGGAC
CTGCTGCGGAACCTCCTGCAGGTAGATCTCACCAAGCGCTTTGGGAACCT
CAAGAATGGGGTCAACGATATCAAGAACCACAAGTGGTTTGCCACAACTG
ACTGGATTGCCATCTACCAGACGAAGGTGGAAGCTCCCTTCATACCAAAG
TTTAAAGGCCCTGGGGATACGAGTAACTTTGACGACTATGAGGAAGAAGA
AATCCGGGTCTCCATCAATGAGAAGTGTGGCAAGGAGTTTTCTGAGTTTT
AGGGGCATGCCTGTGCCCCCATGGGTTTTCTTTTTTCTTTTTTCTTTTTT
TTGGTCGGGGGGGTGGGAGGGTTCGATTGAACAGCCAGAGGGCCCCAGAG
TTCCTTGCATCTAATTTCACCCCCACCCCACCCTCCAGGGTTAGGGGGAG
CAGGAAGCCCAGATAATCAGAGGGACAGAAACACCAGCTGCTCCCCCTCA
TCCCCTTCACCCTCCTGCCCCCTCTCCCACTTTTCCCTTCCTCTTTCCCC
ACAGCCCCCCAGCCCCTCAGCCCTCCCAGCCCACTTCTGCCTGTTTTAAA
CGAGTTTCTCAACTCCAGTCAGACCAGGTCTTGCTGGTGTATCCAGGGAC
AGGGTATGGAAAGAGGGGCTCACGCTTAACTCCAGCCCCCACCCACACCC
CCATCCCACCCAACCACAGGCCCCACTTGCTAAGGGCAAATGAACGAAGC
GCCAACCTTCCTTTCGGAGTAATCCTGCCTGGGAAGGAGAGATTTTTAGT
GACATGTTCAGTGGGTTGCTTGCTAGAATTTTTTTAAAAAAACAACAATT
TAAAATCTTATTTAAGTTCCACCAGTGCCTCCCTCCCTCCTTCCTCTACT
CCCACCCCTCCCATGTCCCCCCATTCCTCAAATCCATTTTAAAGAGAAGC
AGACTGACTTTGGAAAGGGAGGCGCTGGGGTTTGAACCTCCCCGCTGCTA
ATCTCCCCTGGGCCCCTCCCCGGGGAATCCTCTCTGCCAATCCTGCGAGG
GTCTAGGCCCCTTTAGGAAGCCTCCGCTCTCTTTTTCCCCAACAGACCTG
TCTTCACCCTTGGGCTTTGAAAGCCAGACAAAGCAGCTGCCCCTCTCCCT
GCCAAAGAGGAGTCATCCCCCAAAAAGACAGAGGGGGAGCCCCAAGCCCA
AGTCTTTCCTCCCAGCAGCGTTTCCCCCCAACTCCTTAATTTTATTCTCC
GCTAGATTTTAACGTCCAGCCTTCCCTCAGCTGAGTGGGGAGGGCATCCC
TGCAAAAGGGAACAGAAGAGGCCAAGTCCCCCCAAGCCACGGCCCGGGGT
TCAAGGCTAGAGCTGCTGGGGAGGGGCTGCCTGTTTTACTCACCCACCAG
CTTCCGCCTCCCCCATCCTGCGCGCCCCTCCTCCAGCTTAGCTGTCAGCT
GTCCATCACCTCTCCCCCACTTTCTCATTTGTGCTTTTTTCTCTCGTAAT
AGAAAAGTGGGGAGCCGCTGGGGAGCCACCCCATTCATCCCCGTATTTCC
CCCTCTCATAACTTCTCCCCATCCCAGGAGGAGTTCTCAGGCCTGGGGTG
GGGCCCCGGGTGGGTGCGGGGGCGATTCAACCTGTGTGCTCCGAAGGACG
AGACTTCCTCTTGAACAGTGTGCTGTTGTAAACATATTTGAAAACTATTA
CCAATAAAGTTTTGTTTAAAAAAAAAAAAAAAAAA
Human PRKACA mRNA sequence - var2
(public gi: 8489237)

(SEQ ID NO: 106)

GGTGCCCTGAGAACAGGACTGAGTGATGGCTTCCAACTCCAGCGATGTGA
AAGAATTCTTAGCCAAAGCCAAAGAAGATTTTCTTAAAAAATGGGAAAGT
CCCGCTCAGAACACAGCCCA
Human PRKACA mRNA sequence - var3
(public gi: 4506054)

(SEQ ID NO: 107)

CAGTGNGCTCCGGGCCGCCGGCCGCAGCCACCACCCGCCGCGCCGCAGCT
CCCGGACCGGCCCCGGCCGCCGCCGCCGCGATGGGCAACGCCGCCGCCGC
CAAGAAGGGCAGCGAGCAGGAGAGCGTGAAAGAATTCTTAGCCAAAGCCA
AAGAAGATTTTCTTAAAAAATGGGAAAGTCCCGCTCAGAACACAGCCCAC
TTGGATCAGTTTGAACGAATCAAGACCCTCGGCACGGGCTCCTTCGGGCG
GGTGATGCTGGTGAAACACAAGGAGACCGGGAACCACTATGCCATGAAGA
TCCTCGACAAACAGAAGGTCGTCAAACTGAAACAGATCGAACACACCCTG
AATGAAAAGCGCATCCTGCAACCTGTCAACTTTCCGTTCCTCGTCAAACT
CGAGTTCTCCTTCAAGGACAACTCAAACTTATACATGGTCATGGAGTACG
TGCCCGGCGGGGAGATGTTTTCACACCTACGGCGGATCGGAAGGTTCAGT
GAGCCCCATGCCCGTTTCTACGCGGCCCACATCGTCCTGACCTTTGAGTA
TCTGCACTCGCTGGATCTCATCTACAGGGACCTGAAGCCGGAGAATCTGC
TCATTGACCAGCAGGGCTACATTCAGGTGACAGACTTCGGTCTCGCCAAG
CGCGTGAACGGCCGCACTTGGACCTTGTGCGGCACCCCTGAGTACCTGGC
CCCTGAGATTATCCTGAGCAAAGGCTACAACAAGGCCGTGGACTGGTGGG
CCCTGGGGGTTCTTATCTATGAAATGGCCGCTCGCTACCCGCCCTTCTTC
GCAGACCAGCCCATCCAGATCTATGAGAAGATCGTCTCTGGGAAGGTGCG
CTTCCCTTCCCACTTCAGCTCTGACTTGAAGGACCTGCTGCGGAACCTCC
TGCAGGTAGATCTCACCAAGCGCTTTGGGAACCTCAAGAATGGGGTCAAC
GATATCAAGAACCACAAGTGGTTTGCCACAACTGACTGGATTGCCATCTA
CCAGACGAAGGTGGAAGCTCCCTTCATACCAAAGTTTAAAGGCCCTGGGG
ATACGAGTAACTTTGACGACTATGAGGAAGAAGAAATCCGGGTCTCCATC
AATGAGAAGTGTGGCAACGAGTTTTCTGAGTTTAGGGGCATGCCTGTGCC
CCCATGGGTTTTCTTTTCTTTTTTCTTTTTTTTTTTGGTCGGGGGGGTGG
GACGGTTGGATTGAACAGCCAGAGGGCCCCAGAGTTCCTTGCATCTAATT
TCACCCCCACCCCACCCTCCAGGGTTAGGGGGACCAGGAAGCCCAGATAA
TCAGAGGGACAGAAACACCAGCTGCTCCCCCTCATCCCCTTCACCCTCCT
GCCCCCTCTCCCACTTTTCCCTTCCTCTTTCCCCACAGCCCCCCAGCCCC
TCAGCCCTCCCAGCCCACTTCTGCCTGTTTTAAACGAGTTTCTCAACTCC
AGTCAGACCAGGTCTTGCTGGTCTATCCAGGGACAGGGTATGGAAAGAGG
GGCTCACGCTTAACTCCAGCCCCCACCCACACCCCCATCCCACCCAACCA
CACGCCCCACTTGCTAAGCGCAAATGAACGAAGCGCCAACCTTCCTTTCG
GAGTAATCCTGCCTGGGAAGGAGAGATTTTTAGTGACATGTTCAGTGGGT
TGCTTGCTAGAATTTTTTTAAAAAAACAACAATTTAAAATCTTATTTAAG
TTCCACCAGTGCCTCCCTCCCTCCTTCCTCTACTCCCACCCCTCCCATGT
CCCCCCATTCCTCAAATCCATTTTAAAGAGAAGCAGACTGACTTTGGAAA
GGGAGGCGCTGGGGTTTGAACCTCCCCGCTGCTAATCTCCCCTGGGCCCC
TCCCCGGGGAATCCTCTCTGCCAATCCTGCGAGGGTCTACGCCCCTTTAG
GAAGCCTCCGCTCTCTTTTTCCCCAACAGACCTGTCTTCACCCTTGGGCT
TTGAAAGCCAGACAAAGCAGCTGCCCCTCTCCCTGCCAAAGAGGAGTCAT
CCCCCAAAAAGACAGACGGGGAGCCCCAAGCCCAAGTCTTTCCTCCCAGC
AGCGTTTCCCCCCAACTCCTTAATTTTATTCTCCGCTAGATTTTAACGTC
CAGCCTTCCCTCAGCTGAGTGGGGAGGGCATCCCTGCAAAAGCGAACAGA
AGAGGCCAAGTCCCCCCAAGCCACGGCCCGGGGTTCAAGGCTAGAGCTGC
TGGGGAGGGGCTGCCTGTTTTACTCACCCACCAGCTTCCGCCTCCCCCAT
CCTGGGCGCCCCTCCTCCAGCTTAGCTGTCAGCTGTCCATCACCTCTCCC
CCACTTTCTCATTTGTGCTTTTTTCTCTCGTAATAGAAAAGTGGGGAGCC
GCTGGGGAGCCACCCCATTCATCCCCGTATTTCCCCCTCTCATAACTTCT
CCCCATCCCAGGAGGAGTTCTCAGGCCTGGGGTGGGGCCCCGGGTGGGTG
CGGGGGCGATTCAACCTGTGTGCTGCGAAGGACGAGACTTCCTCTTGAAC
AGTGTGCTGTTGTAAACATATTTGAAAACTATTACCAATAAAGTTTGTT
Human PRKACA mRNA sequence - var4
(public gi: 189966)

(SEQ ID NO: 108)

GAATTCTTAGCCAAAGCCAAAGAAGATTTTCTTAAAAAATGGGAAAGTCC
CGCTCAGAACACAGCCCACTTGGATCAGTTTGAACGAATCAAGACCCTCG
GCACGGGCTCCTTCGGGCGGGTGATGCTCGTGAAACACAAGGAGACCGGG
AACCACTATGCCATGAAGATCCTCGACAAACAGAAGGTCGTGAAACTGAA
ACAGATCGAACACACCCTGAATCAAAAGCGCATCCTGCAAGCTGTCAACT
TTCCGTTCCTCGTCAAACTCGAGTTCTCCTTCAAGGACAACTCAAACTTA
TACATGGTCATGGAGTACGTGCCCGGCGGGGAGATGTTCTCACACCTACG
GCGGATCGGAAGGTTCAGTGAGCCCCATGCCCGTTTCTACGCGGCCCAGA
TCGTCCTGACCTTTGAGTATCTGCACTCGCTGGATCTCATCTACAGGGAC
CTGAAGCCGGAGAATCTGCTCATTGACCAGCAGGGCTACATTCAGGTGAC
AGACTTCGGTTTCGCCAAGCGCGTGAAGGGCCGCACTTGGACCTTGTGCG
GCACCCCTGAGTACCTGGCCCCTGAGATTATCCTGAGCAAAGTAGGAGCC
TCCCCAGCCCTCCCCTTCCCCTGAGGCCGGCTCTGCTCTCCTGCTCTCGC
CTCCTCCTCACCCTGTCCCCCCCCATCTTGCTCCAGGGCTACAACAAGGC
CGTGGACTGGTGGGCCCTGGGGGTTCTTATCTATGAAATCGCCGCTCGCT
ACCCGCCCTTCTTCGCAGACCAGCCCATCCAGATCTATGAGAAGATCGTC
TCTGGGAAGGTGAGGTCCGGATGTGGGACACAGCCCTGGAAGAAACAGAC
CGTTCCCTGCTCACCCATCCTATTCCCTGGCGACCCCTGCTTGTTGTCAG
AATAATCTAGAGTTCCTTAAAAAAAAAAAAAAAAAA
Human PRKACA mRNA sequence - var5
(public gi: 11493950)

(SEQ ID NO: 109)

TGAGAACAGGACTGAGTGATGGCTTCCAACTCCAGCGATGTGAAAGAATT
CTTAGCCAAAGCCAAAGAAGATTTTCTTAAAAAATGGGAAAGTCCCGCTC
AGAACACAGCCCACTTGGATCAGTTTGAACGAATCAAGACCCTCGGCACG
GGCTCCTTCGGGCGGGTGATGCTGGTGAAACACAAGGAGACCGGGAACCA
CTATGCCATGAAGATCCTCGACAAACAGAAGGTGGTGAAACTGAAACAGA
TCGAACACACCCTGAATGAAAAGCGCATCCTGCAAGCTGTCAACTTTCCG
TTCCTCGTCAAACTCGAGTTCTCCTTCAAGGACAACTCAAACTTATACAT
GGTCATCGAGTACGTGCCCGGCGGGGAGATGTTCTCACACCTACGGCGGA
TCGGAAGGTTCAGTGAGCCCCATGCCCGTTTCTACGCGGCCCAGATCGTC
CTGACCTTTGAGTATCTGCACTCGCTGGATCTCATCTACAGGGACCTGAA
GCCGGAGAATCTGCTCATTGACCAGCAGGGCTACATTCAGGTGACAGACT
TCGGTTTCGC
Human PRKACA mRNA sequence - var6
(public gi: 8568080)

(SEQ ID NO: 110)

CCCAGTGGCCTCTGGGTTGGGTTTCTCTTCCTGCTCCCACCCCACGGCTC
CCTAGCTCCCCCTGCAGGCAGGGTTCTGGGGACAGACAGCCGAACAGACA
CGGCAGGTCTCATGAGCCTTCCCAGCCACCGTAGTGCCCGTGCCCTGAGA
ACAGGACTGAGTGATGGCTTCCAACTCCAGCGATGTGAAAGAATTCTTAG
CCAAAGCCAAAGAAGATTTTCTTAAAAAATGGGAAAGTCCCGCTCAGAAC
ACAGCCCACTTGGATCAGTTTGAACGAATCAAGACCCTCGGCACGGGCTC
CTTCGGGCGGGTGATGCTGGTGAAACACAAGGAGACCGGGAACCACTATG
CCATGAAGATCCTCGACAAACAGAAGGTGGTGAAACTGAAACAGATCGAA
CACACCCTGAATGAAAAGCGCATCCTGCAAGCTGTCAACTTTCCGTTCCT
CGTCAAACTCGAGTTCTCCTTCAAGGACAACTCAAACTTATACATGGTCA
TGGAGTACGTGCCCGGCGGGGAGATGTTCTCACACCTACGGCGGATCGGA
AGGTTCAGTGAGCCCCATGCCCGTTTCTACGCGGCCCAGATCGT
Human PRKACA Protein sequence - var1
(public gi: 189967)

(SEQ ID NO: 118)

EFLAKAKEDFLRKWESPAQNTAHLDQFERIKTLGTGSFGRVMLVKHKETG
NHYAMKILDKQKVVKLKQIEHTLNEKRILQAVNFPFLVKLEFSFKDNSNL
YMVMEYVPGGEMFSHLRRIGRFSEPHARFYAAQIVLTFEYLHSLDLIYRD
LKPENLLIDQQGYIQVTDFGFAKRVKGRTWTLCGTPEYLAPEIILSKVGA
SPALPFP
Human PRKACA Protein sequence - var2
(public gi: 11493951)

(SEQ ID NO: 119)

MASNSSDVKEFLAKAREDFLKKWESPAQNTAHLDQFERIKTLGTGSFGRV
NLVKHKETGNHYAMKILDKQKVVKLKQIEHTLNEKRILQAVNFPFLVKLE
FSFKDNSNLYMVMEYVPGGENFSHLRRIGRFSEPHARFYAAQIVLTFEYL
HSLDLIYRDLKPENLLIDQQGYIQVTDFGFA
Human PRKACA Protein sequence - var3
(public gi: 8568081)

(SEQ ID NO: 120)

MASNSSDVKEFLAKAKEDFLKKWESPAQNTAHLDQFERIKTLGTGSFGRV
MLVKNKETGNHYAMKILDKQKVVKLKQIEHTLNEKRILQAVNFPFLVKLE
FSFKDNSNLYMVMEYVPGGEMFSHLRRIGRFSEPHARFYAAQIV
Human PRKACA Protein sequence - var4
(public gi: 8489238)

(SEQ ID NO: 121)

MASNSSDVKEFLAKAKEDFLKKWESPAQNTA
Human PRKACA Protein sequence - var5
(public gi: 24980836)

(SEQ ID NO: 122)

MGNAAAAKKGSEQESVKEFLAKAKEDFLKKWESPAQNTAHLDQFERIKTL
GTGSFGRVMLVKHKETGNHYAMKILDKQKVVKLKQIEHTLNEKRILQAVN
FPPLVKLEFSFKDNSNLYMVMEYVPGGEMFSHLRRICRFSEPHARFYAAQ
IVLTFEYLHSLDLIYRDLKPENLLIDQQGYIQVTDFGFAKRVKGRTWTLC
GTPEYLAPEIILSKGYNKAVDWWALGVLIYEMAAGYPPFFADQPIQIYEK
IVSGKVRFPSHFSSDLKDLLRNLLQVDLTKRFGNLKNGVNDIKNHKWFAT
TDWIAIYQRKVEAPFIPKFKGPGDTSNFDDYEEEEIRVSINEKCGKEFSE
F
Human PRKACB mRNA sequence - var1
(public gi: 23272312)

(SEQ ID NO: 111)

AGCGGGTCTGCCCGCCGCCGCCACTCCTGCTGCCACCGCCGTCGCCCCCG
CCGCCGCCGCCGCCACTGCTGCTGCCGGTGCTAAGGAGTTCGCTGGAGCC
CTTTCCTCAGACCCGGCCCGGTCTTCGCGCCCGGACTCCTGGCGCCAGCG
CTACGCGCACTCACCGCTCTGACGGGTGCAGACGCGGGAGTTGTCCCAGA
CTGTGGAGTGGCGGGCACGGCCCCAGCTCCCCTTCCGTTCCCTGACCCCT
TCTTGCCATCGCCCCAGACATGGGGAACGCGGCGACCGCCAAGAAAGGCA
GCGAGGTGGAGAGCGTGAAAGAGTTTCTAGCCAAAGCCAAAGAAGACTTT
TTGAAAAAATGGGAGAATCCAACTCAGAATAATCCCGGACTTGAAGATTT
TGAAAGGAAAAAAACCCTTGGAACAGGTTCATTTGGAAGAGTCATGTTGG
TAAAACACAAAGCCACTGAACAGTATTATGCCATGAAGATCTTAGATAAG
CAGAAGGTTGTTAAACTGAAGCAAATAGAGCATACTTTGAATGAGAAAAG
AATATTACAGGCAGTGAATTTTCCTTTCCTTGTTCGACTGGAGTATGCTT
TTAAGGATAATTCTAATTTATACATCGTTATGGAATATGTCCCTGGGGGT
GAAATGTTTTCACATCTAAGAAGAATTGGAAGGTTCAGTGAGCCCCATGC
ACGGTTCTATGCAGCTCAGATAGTGCTAACATTCGAGTACCTCAATTCAC
TAGACATCATCTACAGACATCTAAAACCTGAAAATCTCTTAATTGACCAT
CAAGGCTATATCCAGGTCACAGACTTTGGGTTTGCCAAAAGAGTTAAAGG
CAGAACTTGGACATTATGTGGAACTCCAGAGTATTTGGCTCCAGAAATAA
TTCTCAGCAAGGGCTACAATAAGGCAGTGGATTGGTGGGCATTAGGAGTG
CTAATCTATGAAATGGCAGCTGGCTATCCCCCATTCTTTGCAGACCAACC
AATTCAGATTTATGAAAAGATTGTTTCTGGAAAGGTCCGATTCCCATCCA
ACTTCAGTTCAGATCTCAAGGACCTTCTACGGAACCTGCTGCAGGTGGAT
TTGACCAAGAGATTTGGAAATCTAAAGAATGGTGTCAGTGATATAAAAAC
TCACAAGTGGTTTGCCACGACAGATTGGATTGCTATTTACCAGAGGAAGG
TTGAAGCTCCATTCATACCAAAGTTTAGAGGCTCTGGAGATACCAGCAAC
TTTGATGACTATGAAGAAGAAGATATCCGTGTCTCTATAACAGAAAAATG
TGCAAAAGAATTTGCTCAATTTTAAAGAGGAACAAGATGACATCTGACCT
CACACTCAGTGTTTGCACTCTGTTGAGAGATAAGGTAGAGCTGAGACCGT
CCTTGTTGAAGCAGTTACCTAGTTCCTTCATTCCAACGACTGAGTGAGGT
CTTTATTGCCATCATCCCGTGTGCGCACTCTGCATCCACCTATGTAACAA
GGCACCGCTAAGCAAGCATTGTCTGTGCCATAACACAGTACTAGACCACT
TTCTTACTTCTCTTTGGGTTGTCTTTCTCCTCTCCTATATCCATTTCTTC
CTTTTCCAATTTCATTGGTTTTCTCTAAACAGTGCTCCATTTTATTTTGT
TCGTGTTTCAGATGGGCAGTGTTATGGCTACGTGATATTTGAACGGAAGG
ATAAGTGTTGCTTTCAGTAGTTATTGCCAATATTGTTGTTGGTCAATGGC
TTGAAGATAAACTTTCTAATAATTATTATTTCTTTGAGTAGCTCAGACTT
GGTTTTGCCAAAACTCTTGGTAATTTTTGAAGATAGACTGTCTTATCACC
AAGGAAATTTATACAAATTAAGACTAACTTTCTTGGAATTCACTATTCTG
GCAATAAATTTTGGTAGACTAATACAGTACAGCTAGACCCAGAAATTTGG
AACGCTGTAGATCAGAGGTTCTAGTTCCCTTTCCCTCCTTTTATATCCTC
CTCTCCTTGAGTAATGAAGTGACCAGCCTGTGTAGTGTGACAAACGTGTC
TCATTCAGCAGGAAAAACTAATGATATGGATCATCACCCAGATTCTCTCA
CTTGGTACCAGCATTTCTGTACGTATTAGAGAAGAGTTCTAAGTTTTCTA
AACCTTAACTGTTCCTTAAGGATTTTAGCCAGTATTTTAATAGAACATGA
TTAATGAAAGTGACAAATTTTAAATTTTCTCTAATAGTCCTCATCATAAA
CTTTTTAAAGGAAAATAACCAAACTAAAAAGAACATTGGTTTAGATAAAT
ACTTATACTTTGCAAAGTCAAAAATGCCTTGATTTTTGGAAACAATATAG
AGGTATTCATATTTAAATGAGGGTTTACATTTGTTTTGTTTTGTAACCGT
TAAAAAGAAGTTGTTTCCAGCTAATTATTGTGGTGTACTATATTTGTGAG
CCTAGGGTAGGGGCACTGCTCCAACTTCTGCTTTCATCCCATGCCTCATC
AATGAGGAAAGGGAACAAAGTGTATAAAACTGCCACAATTGTATTTTAAT
TTTGAGGTATGATATTTTCAGATATTTCATAATTTCTAACCTCTGTTCTC
TCAGTAAACAGAATGTCTGATCGATCATGCAGATACAATGTTGGTATTTG
AGAGGTTAGTTTTTTTCCTACACTTTTTTTTGCCAACTGACTTAACAACA
TTGCTGTCAGGTGGAAATTTCAAGCACTTTTGCACATTTAGTTCAGTGTT
TGTTGAGAATCCATGGCTTAACCCACTTGTTTTGCTATTTTTTTCTTTGC
TTTTAATTTTCCCCATCTGATTTTATCTCTGCGTTTCAGTGACCTACCTT
AAAACAACACACGAGAAGAGTTAAACTGGGTTCATTTTAATGATCAATTT
ACCTGCATATAAAATTTATTTTTAATCAAGCTGATCTTAATGTATATAAT
CATTCTATTTGCTTTATTATCGGTGCAGGTAGGTCATTAACACCACTTCT
TTTCATCTGTACCACACCCTGGTGAAACCTTTGAAGACATAAAAAAAACC
TGTCTGAGATGTTCTTTCTACCAATCTATATGTCTTTCCGTTATCAAGTG
TTTCTGCATGGTAATCTCATGTAAATGCTGATATTGATTTCACTGGTCCA
TCTATATTTAAAACGTGCAACAAAAAAATAAAATACTCTGCTCTAGCAAG
TTTTGTGTAACAAAGGCATATCGTCATGTTAATAAATTTAAAACATCATT
CGTATAAAATATTTTAATTTTCTTGTATTTCATTTAGACCCAAGAACATG
TTGACCAATGTGTTCTATATGTAAACTACAAATTCTATGGTAGCTTTGTT
GTATATTATTGTAAAATTATTTTAATAAGTCATGGGGATGACAATTTGAT
TATTACAATTTAGTTTTCAGTAATCAAAAAGATTTCTATGAATTCTAAAA
AATATTTTTTTCTATGAAATTACTAGTGCCCAGCTGTAGAATCTACCTTA
GGTAGATGATCCCTAGACATACGTTGGTTTTGAGGGCTATTCAGCCATTC
CATTTTACTCTCTATTTAAAGGCCGTGAGCAAGCTTGTCATGAGCAAATA
TGTCAAGGGAGTCAATCTCTGACCAATCAAGTACACTAAATTAGAATATT
TTTAAAGTATGTAACATTCCCAGTTTCAGCCACAATTTAGCCAAGAATAA
GATAAAAACTTGAATAAGAAGTAAGTAGCATAAATCAGTATTTAACCTAA
AATTACATATTTGAAACAGAAGATATTATGTTATGCTCAGTAAATAATTA
AGAGATGGCATTGTGTAAGAAGGAGCCCTAGACTGAAAGTCAAGACATCT
GAATTTCAGGCTGGAAAACTATCAGTATGATCTCAGCCTCAGTTCTCTTG
TCTGTAAGATGGAAGAACTGGATTAGGCAGTTTGTAAGATTCCTCCTAAC
TTTCACAGTCCATGACAAGATTGTCTTTTTATCTGATATTTTGAAGGGTA
TATTGCTTTGAAGTAAGTCTCAATAAGGCAATATATTTTAGGGCATCTTT
CTTCTTATCTCTGACAGTGTTCTTAAAATTATTTGAATATCATAAGAGCC
TTGGTGTCTGTCCTAATTCCTTTCTCACTCACCGATGCTGAATACCCAGT
TGAATCAAACTGTCAACCTACCAAAAACGATATTGTGGCTTATGGGTATT
GCTGTCTCATTCTTGGTATATTCTTGTGTTAACTGCCCATTGGCCTGAAA
ATACTCATTGTAAGCCTGAAAAAAAAAATCTTTCCCACTGTTTTTTCTGC
TTGTTGTAAGAATCAAATGAAATAATGTATGTGAAAGCACCTTGTAAACT
GTAACCTATCAATGTAAAATGTTAAGGTGTGTTGTTATTTCATTAATTAC
TTCTTTGTTTAGAATGGAATTTCCTATGCACTACTGTAGCTAGGAAATGC
TGAAAACAACTGTGTTTTTTAATTAATCAATAACTGCAAAATTAAAGTAC
CTTCAATGGATAAGACAACAAAAAAAAAAAAAAAA
Human PRKACB mRNA sequence - var2
(public gi: 4884447)

(SEQ ID NO: 112)

AAAAAAAATCTTTCCCACTGTTTTTTCTGCTTGTTGTAAGAATCAAATGA
AATAATGTATGTGAAAGCACCTTGTAAACTGTAACCTATCAATGTAAAAT
GTTAAGGTGTGTTGTTATTTCATTAATTACTTCTTTGTTTAGAATGGAAT
TTCCTATGCACTACTGTAGCTAGGAAATGCTGAAAACAACTGTGTTTTTT
AATTAATCAATAACTGCAAAATTAAAGTACCTTCAATGGATAAGACAAAA
AAAAAAAAAAAAAAAAAAAAAAAAAAAAAAAAAAAAAAAAAAAAAA
Human PRKACB mRNA sequence - var3
(public gi: 21749785)

(SEQ ID NO: 113)

GTTATTTTGAGCAATATGTTTTGGAAAGGTTGGTTTTCATCATGAGTGCA
CGCAAATCATCAGATGCATCTCCTTGCTCCTCTTCAGAAATATCTGTGAA
AGAGTTTCTAGCCAAAGCCAAAGAAGACTTTTTGAAAAAATCGGAGAATC
CAACTCAGAATAATGCCGGACTTGAAGATTTTGAAAGGAAAAAAACCCTT
GGAACAGGTTCATTTGGAAGAGTCATGTTGGTAAAACACAAAGCCACTGA
ACAGTATTATGCCATGAAGATCTTAGATAACCAGAAGGATAATTCTAATT
TATACATGGTTATGGAATATGTCCCTGGGGGTGAAATGTTTTCACATCTA
AGAAGAATTGGAAGGTTCAGTGAGCCCCATGCACGGTTCTATGCAGCTCA
GATAGTGCTAACATTCGAGTACCTCCATTCACTAGACCTCATCTACAGAG
ATCTAAAACCTGAAAATCTCTTAATTGACCATCAACGCTATATCCAGGTC
ACAGACTTTGGGTTTGCCAAAAGAGTTAAAGGCAGAACTTGGACATTATG
TGGAACTCCAGAGTATTTGGCTCCAGAAATAATTCTCAGCAAGGGCTACA
ATAAGGCAGTGGATTGGTGGGCATTAGGAGTGCTAATCTATGAAATGGCA
GCTGGCTATCCCCCATTCTTTGCAGACCAACCAATTCAGATTTATGAAAA
GATTGTTTCTGGAAAGGTCCGATTCCCATCCCACTTCAGTTCAGATCTCA
AGGACCTTCTACGGAACCTGCTGCAGGTGGATTTGACCAAGAGATTTGGA
AATCTAAAGAATGGTGTCAGTGATATAAAAACTCACAAGTGCTTTGCCAC
GACAGATTGGATTGCTATTTACCAGAGGAAGGTTGAAGCTCCATTCATAC
CAAAGTTTAGAGGCTCTGGAGATACCAGCAACTTTGATGACTATGAAGAA
GAAGATATCCGTGTCTCTATAACAGAAAAATGTGCAAAAGAATTTGGTGA
ATTTTAAAGAGGAACAAGATGACATCTGAGCTCACACTCAGTGTTTGCAC
TCTGTTGAGAGATAAGGTAGAGCTGAGACCGTCCTTGTTGAAGCAGTTAC
CTAGTTCCTTCATTCCAACGACTGAGTGAGGTCTTTATTGCCATCATCCC
GTGTGCGCACTCTGCATCCACCTATGTAACAAGGCACCGCTAAGCAAGCA
TTGTCTGTGCCATAACACAGTACTAGACCACTTTCTTACTTCTCTTTGGG
TTGTCTTTCTCCTCTCCTACATCCATTTCTTCCTTTTCCAATTTCATTGG
TTTTCTCTAAACAGTGCTCCATTTTATTTTGTTGGTGTTTCAGATGGGCA
GTGTTATGGCTACGTGATATTTGAAGGGAAGGATAAGTGTTGCTTTCAGT
AGTTATTGCCAATATTGTTGTTGGTCAATGGCTTGAAGATAAACTTTCTA
ATAATTATTATTTCTTTGAGTAGCTCAGACTTGGTTTTGCCAAAACTCTT
GGTAATTTTTGAAGATAGACTGTCTTATCACCAAGGAAATTTATACAAAT
TAAGACTAACTTTCTTGGAATTCACTATTCTGGCAATAAATTTTGGTAGA
CTAATACAGTACAGCTAGACCCAGAAATTTGGAAGGCTGTAGATCAGAGG
TTCTAGTTCCCTTTCCCTCCTTTTATATCCTCCTCTCCTTGAGTAATGAA
GTGACCAGCCTGTGTAGTGTGACAAACGTGTCTCATTCAGCAGGAAAAAC
TAATGATATGGATCATCACCCAGATTCTCTCACTTGGTACCAGCATTTCT
GTAGGTATTAGAGAAGAGTTCTAAGTTTTCTAAACCTTAACTGTTCCTTA
AGGATTTTAGCCAGTATTTTAATAGAACATGATTAATGAAAGTGACAAAT
TTTAAATTTTCTCTAATAGTCCTCATCATAAACTTTTTAAACGAAAATAA
GCAAACTAAAAAGAACATTGGTTTAGATAAATACTTATACTTTGCAAAGT
CAAAAATCGCTTGATTTTTGGAAACAATATAGAGGTATTCATATTTAAAT
GAGGGTTTACATTTGTTTTGTTTTGTAACCGTTAAAAAGAAGTTGTTTCC
AGCTAATTATTGTGGTGTACTATATTTGTGAGCCTAGGGTAGGGGCACTG
CTCCAACTTCTGCTTTCATCCCATGCCTCATCAATGAGGAAAGGGAACAA
AGTGTATAAAACTGCCACAATTGTATTTTAATTTTGAGGTATGATATTTT
CAGATATTTCATAATTTCTAACCTCTGTTCTCTCAGTAAACAGAATGTCT
GATCGATCATGCAGATACAATGTTGGTATTTGAGAGGTTAGTTTTTTTCC
TACACTTTTTTTTGCCAACTGACTTAACAACATTGCTGTCAGGTGGAAAT
TTCAAGCACTTTTGCACATTTAGTTCAGTGTTTGTTGAGAATCCATGGCT
TAACCCACTTGTTTTGCTATTTTTTTCTTTGCTTTTAATTTTCCCCATCT
GATTTTATCTCTGCGTTTCAGTGACCTACCTTAAAACAACACACGAGAAG
AGTTAAACTGGGTTCATTTTAATGATCAATTTACCTGCATATAAAATTTA
TTTTTAATCAAGCTGATCTTAATGTATATAATCATTCTATTTGCTTTATT
ATCGGTGCAGGTAGGTCATTAACACCACTTCTTTTCATCTGTACCACACC
CTGGTGAAACCTTTGAAGACATAAAAAAAACCTGTCTGAGATGTTCTTTC
TACCAATCTATATGTCTTTCGGTTATCAAGTGTTTCTGCATGGTAATGTC
ATGTAAATGCTGATATTGATTTCACTGGTCCATCTATATTTAAAACGTGC
Human PRKACB mRNA sequence - var4
(public gi: 16740847)

(SEQ ID NO: 114)

GTTCGCTGGAGCCCTTTCCTCAGACCCGGCCCGGTCTTCGCGCCCGGACT
CCTGGCGCCAGCGCTAGGCGCACTCACCGCTCTGACGGGTGCAGACGCGG
GAGTTGTCCCAGACTGTGGAGTGGCGGGCACGGCCCCAGCCCCCCTTCCC
TTCCCTGACCCCTTCTTGCCATCGCCCCAGACATGGCGAACGCGGCGACC
GCCAAGAAAGGCAGCGAGGTGGAGAGCGTGAAAGAGTTTCTAGCCAAAGC
CAAAGAAGACTTTTTGAAAAAATGGGAGAATCCAACTCAGAATAATGCCG
GACTTGAAGATTTTGAAAGGAAAAAAACCCTTGGAACAGGTTCATTTGGA
AGAGTCATGTTGGTAAAACACAAAGCCACTGAACACTATTATGCCATGAA
GATCTTAGATAAGCAGAAGGTTGTTAAACTGAACCAAATAGAGCATACTT
TGAATGAGAAAAGAATATTACAGGCAGTGAATTTTCCTTTCCTTGTTCGA
CTGGACTATGCTTTTAAGGATAATTCTAATTTATACATGGTTATGGAATA
TGTCCCTGGGGGTGAAATGTTTTCACATCTAAGAAGAATTGGAAGGTTCA
GTGAGCCCCATCCACGGTTCTATGCAGCTCAGATAGTGCTAACATTCGAG
TACCTCCATTCACTAGACCTCATCTACAGAGATCTAAAACCTCAAAATCT
CTTAATTGACCATCAAGGCTATATCCAGGTCACAGACTTTGGGTTTGCCA
AAAGAGTTAAAGGCAGAACTTGGACATTATGTGGAACTCCAGAGTATTTG
GCTCCAGAAATAATTCTCAGCAAGGGCTACAATAAGGCAGTGGATTGGTC
GGCATTAGGAGTGCTAATCTATGAAATGGCAGCTGGCTATCCCCCATTCT
TTGCAGACCAACCAATTCAGATTTATGAAAAGATTGTTTCTGGAAAGAAC
TTTTGATATGAACAAAACAAAACTTTGAGAAAAATTAACAGACAAGGCAG
TGATTTATTTTTGAAGAATTTGACAAGTGTAGACTCTCAAGAGGACTAAA
GGTCATATGAAGAATGATGAGAGAACCAAAATACATTAAAATCACAAATG
GAAGAAGAATATTTTACTAATACAAAAACTAAGAATGTAAATGTTATAAT
AATTGTTTCAAATCATTTAATTGACAGTAATTATAAAGTTCTTGAATCTT
TACTATATTACTTTTATTTATACTTCATATAAGAAATCCAGTTTTCTAAC
AAGGATACTGTCATAACTAAATTTACATTTATTAAGAAAAACTGCTTTAG
TTAAAATTAATGTGTCTTCATTTTTATGCATTGGCCTCGATTTGCCAATC
ATTCTCTATTGGTTAAAATTTATATTCAGCTGTTTATGAATATATATTCA
TTTTATATCAAACTTTAAAATTTTGTATCTAATAATCAGCATATATTCTA
AAATCATAACAGTCTAAATCCTGGGCACCTTAGAAGAATGACACCAGAAA
ACCTTATTATATCACAATATTCTGTTTTCCCCTTCATTTATTTAGAAATA
TGACAGGATATTTGGTGTACTTTTGTTTTTTAACTAAAAGTACCAGATTC
TCTCTCCCCATGTGGGATATAAAATTATCCCCATCTCTTACTCCCTTTAC
TCATCTAAAGTAGAAGTCATGAAAGTGGAATTTTTCCCATTAAAACGCTC
TGTATTATGTGAAGTTAGATTGTATTAACCATTTCCCAATAAATCATCTG
TTTCAAAACTCAAATTCAAACTAGAATCTGTCTCTATTCACATTGCAAAA
ATATTATTGTCTCTCTGGTTAGTGGCTAAAAGCCAAATTGGAAACTAACT
AGTTTTTTAAATTTTTTAAATTGTGCAAATTATTAAAAATCCAATTTGGT
CTTATAAAAAAAAAAAAAAAAAAAAAAAAAAAAAAAAAAAAAAAAAAAAA
A
Human PRKACB mRNA sequence - var5
(public gi: 189982)

(SEQ ID NO: 115)

CCAGCCCCCCTTCCCTTCCCTGACCCCTTCTTGCCATCGCCCCAGACATG
GGGAACGCGGCGACCGCCAAGAAAGGCAGCGAGGTGGAGACCGTGAAAGA
GTTTCTAGCCAAAGCCAAAGAAGACTTTTTGAAAAAATGGGAGAATCCAA
CTCAGAATAATGCCGGACTTGAAGATTTTGAAAGGAAAAAAACCCTTGGA
ACAGGTTCATTTGGAAGAGTCATGTTCGTAAAACACAAAGCCACTGAACA
GTATTATGCCATGAAGATCTTAGATAAGCAGAAGGTTGTTAAACTGAAGC
AAATAGAGCATACTTTGAATGAGAAAAGAATATTACAGGCAGTGAATTTT
CCTTTCCTTGTTCGACTGGAGTATGCTTTTAAGGATAATTCTAATTTATA
CATGGTTATGGAATATGTCCCTGGGGGTGAAATGTTTTCACATCTAAGAA
GAATTGGAAGGTTCAGTGAGCCCCATGCACGGTTCTATGCAGCTCAGATA
GTGCTAACATTCGAGTACCTCCATTCACTAGACCTCATCTACAGAGATCT
AAAACCTGAAAATCTCTTAATTGACCATCAAGGCTATATCCAGGTCACAG
ACTTTGGGTTTGCCAAAAGAGTTAAAGGCAGAACTTGGACATTATGTGGA
ACTCCAGAGTATTTGGCTCCAGAAATAATTCTCAGCAAGGGCTACAATAA
GGCAGTGGATTGGTGGGCATTAGGACTGCTAATCTATGAAATGGCAGCTG
GCTATCCCCCATTCTTTGCAGACCAACCAATTCAGATTTATGAAAAGATT
GTTTCTGGAAAGGTCCGATTCCCATCCCACTTCAGTTCAGATCTCAAGGA
CCTTCTACGGAACCTGCTGCAGGTGGATTTGACCAAGAGATTTGGAAATC
TAAAGAATGGTGTCAGTGATATAAAAACTCACAAGTGGTTTGCCACGACA
GATTGGATTGCTATTTACCAGAGGAAGGTTGAAGCTCCATTCATACCAAA
GTTTAGAGGCTCTGGAGATACCAGCAACTTTGATGACTATGAAGAAGAAG
ATATCCGTGTCTCTATAACAGAAAAATGTGCAAAAGAATTTGGTGAATTT
TAAAGAGGAACAAGATGACATCTGAGCTCACACTCAGTGTTTGCACTCTG
TTGAGAGATAAGGTACACCTGAGACCGTCCTTGTTGAAGCAGTTACCTAG
TTCCTTCATTCCAACGACTCAGTGAGGTCTTTATTGCCATCATCCGTGTG
CGCACTCTGCATCCACCTATGTAACAAGGCACCGCTAAGCAAGCATTGTC
TGTGCCATAACACAGTACTAGACCACTTTCTTACTTCTCTTTGGGTTGTC
TTTCTCCTCTCCTACATCCATTTCTTCCTTTTCAATTTCATTGGTTTTCT
CTAAACAGTGCTCCATTTTATTTTGTTGGTGTTTCAGATGGGCAGTGTTA
TGGCTACGTGATATTTGAAGCGAACGATAAGTGTTCCTTTCACTAGTTAT
TGCCAATATTGTTGTTGGTCAATGGCTTGAAGATAAACTTTCTAATAATT
ATTATTTCTTTGAGTAGCTCAGACTTGGTTTTGCCAAAACTCTTGGTAAT
TTTTGAAGATAGACTGTCTTATCACCAAGGAAATTTATACAAATTAAGAC
TAACTTTCTTGGAATTCACTATTCTGGCAATAAATTTTGGTAGACTAATA
CAGTACAGCTAGACCCAGAAATTTGGAAGGCTGTAGATCAGAGGTTCTAG
TTCCCTTTCCCTCCTTTTATATCCTCCTCTCCTTGAGTAATGAAGTGACC
AGCCTGTGTAGTGTGACAAACGTGTCTCATTCACCAGGAAAAACTAATGA
TATGGATCATCACCCAGATTCTCTCACTTCGTACCAGCATTTCTGTAGGT
ATTAGAGAAGAGTTCTAAGTTTTCTAAACCTTAAGTGTTCCTTAAGGATT
TTACCCAGTATTTTAATAGAACATGATTAATGAAAGTGACAAATTTTAAA
TTTTCTCTAATAGTCCTCATCATAAACTTTTTAAAGGAAAATAAGCAAAC
TAAAAAGAACATTGGTTTAGATAAATACTTATACTTTGCAAAGTCAAAAA
TGGCTTGATTTTTGGAAACAATATAGAGGTATTCATATTTAAATGAGGGT
TTACATTTGTTTTGTTTTGTAACCGTTAAAAAGAAGTTGTTTCCAGCTAA
TTATTGTGGTGTACTATATTTGTGAGCCTAGGGTAGGGGCACTGCTGCAA
CTTCTGCTTTCATCCCATGCCTCATCAATGAGGAAAGGGAACAAAGTGTA
TAAAACCTGCCACAATTGTATTTTAATTTTGAGGTATGATATTTTCAGAT
ATTTCATAATTTCTAACCTCTGTTCTCTCAGTAAACAGAATGTCTGATCG
ATCATGCAGATACAATGTTGGTATTTGAGAGGTTAGTTTTTTTCCTACAC
TTTTTTTTGCCAACTGACTTAACAACATTGCTGTCAGGTGGAAATTTCAA
GCACTTTTGCACATTTAGTTCAGTGTTTGTTGAGAATCCATGGCTTAACC
CACTTGTTTTGCTATTTTTTTCTTTGCTTTTAATTTTCCCCATCTGATTT
TATCTCTGCGTTTCAGTGACCTACCTTAAAACAACACACGAGAAGAGTTA
AACTGGGTTCATTTTAATGATCAATTTACCTGCATATAAAATTTATTTTT
AATCAAGCTGATCTTAATGTATATAATCATTCTATTTGCTTTATTATCGG
TGCAGGTAGGTCATTAACACCACTTCTTTTCATCTGTACCACACCCTCGT
GAAACCTTTGAAGACATAAAAAAAACCTGTCTGAGATGTTCTTTCTACCA
ATCTATATGTCTTTCGGTTATCAAGTGTTTCTGCATGGTAATGTCATGTA
AATGCTGATATTGATTTCACTGGTCCATCTATATTTAAAACGTGC
Human PRKACB Protein sequence - var1
(public gi: 189983)

(SEQ ID NO: 123)

MGNAATAKKGSEVESVKEFLAKAKEDFLKKWENPTQNNAGLEDFERKKTL
GTGSFGRVMLVKHKATEQYYANKILDKQKVVKLKQIEHTLNEKRILQAVN
FPFLVRLEYAFKDNSNLYMVMEYVPGGEMFSHLRRIGRFSEPHARFYAAQ
IVLTFEYLHSLDLIYRDLKPENLLIDHQGYIQVTDFGFAKRVKGRTWTLC
GTPEYLAPEIILSKGYNKAVDWWALGVLIYEMAAGYPPFFADQPIQIYEK
IVSGKVRFPSHFSSDLKDLLRNLLQVDLTKRFGNLKNGVSDIKTHKWFAT
TDWIAIYQRKVFAPFIPKFRGSGDTSNFDDYEEEDIRVSITEKCAKEFGE
F
Human PRKACB Protein sequence - var2
(public gi: 16740848)

(SEQ ID NO: 124)

MGNAATAKKGSEVESVKEFLAKAKEDFLKKWENPTQNNAGLEDFERKKTL
CTGSFGRVMLVKHKATEQYYAMKILDKQKVVKLKQIEHTLNEKRILQAVN
FPFLVRLEYAFKDNSNLYNVMEYVPGGEMFSHLRRIGRFSEPHARFYAAQ
IVLTFEYLHSLDLIYRDLKPENLLIDHQGYIQVTDFGFAKRVKGRTWTLC
GTPEYLAPEIILSKGYNKAVDWWALGVLIYEMAAGYPPFFADQPIQIYEK
IVSGKNF
Human PRKACB Protein sequence - var3
(public gi: 23272313)

(SEQ ID NO: 125)

MGNAATAKKGSEVESVKEFLAKAKEDFLKKWENPTQNNAGLEDFERKKTL
GTGSFGRVMLVKHKATEQYYANKILDKQKVVKLKQIEHTLNEKRILQAVN
FPFLVRLEYAFKDNSNLYMVMEYVPGGEMPSHLRRIGRFSEPHARFYAAQ
IVLTFEYLNSLDIIYRDLKPENLLIDHQGYIQVTDFGFAKRVKGRTWTLC
GTPEYLAPEIILSKGYNKAVDWWALCVLIYEMAAGYPPFFADQPIQIYEK
IVSGKVRFPSNFSSDLKDLLRNLLQVDLTKRFGNLKNGVSDIKTHKWFAT
TDWIAIYQRKVEAPFIPKFRGSGDTSNFDDYEEEDIRVSITEKCAKEFGE
F

Below is the procedure used to clone GOSR2 and RALA:
Bait:

Plasmid vector: pGBK-T7 (Clontech)

Plasmid name: pPL269-pGBK-T7 GAL4 POSHdR

Protein sequence: Corresponds to aa 53-888 of POSH

RTLVGSGVEELPSNILLVRLLDGIKQRPWKPGPGGGSGTNCTNALRSQSS
TVANCSSKDLQSSQGGQQPRVQSWSPPVRGIPQLPCAKALYNYEGKEPGD
LKFSKGDIIILRRQVDENWYHGEVNGIHGFFPTNFVQIIKPLPQPPPQCK
ALYDFEVKDKEADKDCLPFAKDDVLTVIRRVDENWAEGMLADKIGIFPIS
YVEFNSAAKQLIEWDKPPVPGVDAGECSSAAAQSSTAPKHSDTKKNTKKR
HSFTSLTMANKSSQASQNRHSMEISPPVLISSSNPTAAARISELSGLSCS
APSQVHISTTGLIVTPPPSSPVTTGPSFTFPSDVPYQAALGTLNPPLPPP
PLLAATVLASTPPGATAAAAAAGMGPRPMAGSTDQIAHLRPQTRPSVYVA
IYPYTPRKEDELELRKGEMFLVFERCQDGWFKGTSMHTSKIGVFPGNYVA
PVTRAVTNASQAKVPMSTAGQTSRGVTMVSPSTAGGPAQKLQGNGVAGSP
SVVPAAVVSAAHIQTSPQAKVLLHMTGQMTVNQARNAVRTVAAHNQERPT
AAVTPIQVQNAAGLSPASVGLSHHSLASPQPAPLMPGSATHTAAISISRA
SAPLACAAAAPLTSPSITSASLEAEPSGRIVTVLPGLPTSPDSASSACGN
SSATKPDKDSKKEKKGLLKLLSGASTKRKPRVSPPASPTLEVELGSAELP
LQGAVGPELPPGGGHGRAGSCPVDGDGPVTTAVAGAALAQDAFHRKASSL
DSAVPIAPPPRQACSSLGPVLNESRPVVCERHRVVVSYPPQSEAELELKE
GDIVFVHKKREDGWFKGTLQRNGKTGLFPGSFVENI
Library screened: Hela pretransformed library
(Clontech).

Procedure:

Bait plasmid (GAL4-BD) was transformed into yeast strain AH109 (Clontech) and transfroments were selected on defined media lacking tryptophan. Yeast strain Y187 containing pre-transformed Hela cDNA prey(GAL4-AD) library (Clontech) was mated according to Clontech's protocol with bait containing yeast and plated on defined media lacking tryptophan, leucine, histidine and containing 2 mM 3-amino triazol. Colonies that grew on the selective media were tested for beta-galactosidase activity and positive clones were further characterized. Plasmid was recovered from yeast colonies and transformed into E. coli DH5alpha strain. After ampicillin selection plasmid was prepared from bacterial colonies and transformed back into yeast strain AH109 together with bait plasmid or empty bait vector and colonies selected on defined media lacking leucine and tryptophan and then scored for growth on media lacking tryptophan, leucine, histidine and containing 5 mM 3-amino triazol. Only prey clones which their growth on this media was dependent on bait plasmid were scored as true hits. Prey dones were identified by amplifying cDNA insert and sequencing using vector derived primers.

Examples of sequences for the POSH-APs, GOSR2 and RALA, are presented below.

Human GOSR2 mRNA sequence - var1
(public gi: 2316087)

(SEQ ID NO: 63)

ATGGATCCCCTGTTCCAGCAAACGCACAAGCAGGTCCACGAGATCCAGTC
TTGCATGGGACGCCTGGAGACGGCAGACAAGCAGTCTGTGCACATAGTAG
AAAACGAAATCCAAGCAAGCATAGACCAGATATTCAGCCGTCTAGAACGT
CTCGAGATTTTGTCCAGCAAGGAGCCCCCTAACAAAAGGCAAAATGCCAG
ACTTCGGGTTGACCAGTTAAAGTATGATGTCCAGCACCTGCAGACTGCGC
TCAGAAACTTCCAGCATCGGCGCCATGCAAGGGAGCAGCAGGAGAGACAG
CGAGAAGAGCTTCTGTGTCGAACGTTCACCACTAACGGCTCTGACACCAC
CATACCAATGGACGAATCACTGCAGTTTAACTCCTCCCTCCAGAAAGTTC
ACAACGGCATGGATGACCTCATTTTAGATCGGCACAATATTTTAGATGGA
CTGAGGACCCAGAGACTGACCTTGAAGGGGACTCAGAAGAAGATCCCTGA
CATTGCCAACATGCTGGGCTTGTCCAACACAGTGATGCGGCTCATCGAGA
AGCGGGCTTTCCAGGACAAGTACTTTATGATAGGTGGGATGCTGCTGACC
TGTGTGGTCATGTTCCTCGTGGTGCAGTACCTGACATGA
Human GOSR2 mRNA sequence - var2
(public gi: 21961348)

(SEQ ID NO: 64)

GGCCTGCCGGGCCGGCGACATGGATCCCCTGTTCCAGCAAACGCACAAGC
AGGTCCACGAGATCCAGTCTTGCATGGGACGCCTGGAGACGGCAGACAAG
CAGTCTCTGCACATAGTAGAAAACGAAATCCAAGCAAGCATAGACCAGAT
ATTCACCCGTCTAGAACGTCTCGAGATTTTGTCCACCAAGGAGCCCCCTA
ACAAAAGGCAAAATGCCAGACTTCGGGTTGACCAGTTAAAGTATGATGTC
CAGCACCTGCACACTGCGCTCAGAAACTTCCAGCATCGGCGCCATGCAAG
GGAGCAGCAGGAGAGACAGCGAGAAGAGCTTCTGTCTCGAACCTTCACCA
CTAACGACTCTGACACCACCATACCAATGGACGAATCACTGCAGTTTAAC
TCCTCCCTCCAGAAAGTTCACAACGGCATCGATGACCTCATTTTAGATGG
GCACAATATTTTAGATGGACTGAGGACCCAGAGACTGACCTTGAAGGTGG
GGTCCCTGCTGGGGGACAGAGAGAAGOCCTCTTGTTTTAGCCTCATCCAA
CAGTTTACTAACTGTGTTTATATTTTGATTACGTGTCCTCAAATTGTGAT
ATTTTGATGACAAGACAGAGCCCTTGAGTTTGGGATCCTTTCTGTTGGAG
TTGAGTTATTGTGAGCCTGAAAGTACCCAGTTCCTTTGCCAGTGCTTGAA
ACAAACCATGAAGTGGCCTCTCTTAGGATCCAGGTCTTTTCCCATTTACT
GAACTTATCATGAAAGTGAGTGCTACTACGACGGGTCCAATCACAGGCTG
AGAAATTGTGTTACAGAATCTACTCTTGGAAGAATGAAGACGTGGCTGTC
CTTTGGTACCTCGCTTTAAGGTGGCTTTCCCTTAGGACCCCTACTGTGGA
CTGCCTTATAACTAAAACCTTTTGTATTTTAGTAACTGAATCCCCACTGT
GCAGTGTTAGGGCTGCCTGGTTGTTTGCAGTAGATTAGAGCTTTAGAAGC
TTCTAGAGCTTCTAAAGCCCGTGCTGGTGATCCCAGCGACTCTTCACTCC
CTAGCCTTAGGTATTCCTAGAAGCCCTGACCAGTTGGCACTGCTGAGACT
CCAGCCCCTGGGAGTGGTTTACAGAAACATTACACAGACTCTGATGTCAG
TCATGATGTTTCAGCCTCTGCCCTTTTCCTGTATCAACCCTGATGGATAA
TAGGGCGTGGGTTCTGTCTGTTATCAGGGTGTGGTCCCCTGTCAATGAAG
CACTCCCAGCCACTGAGCTGTGAGAAACAGTCACTCGGAAGTGTGAGCTT
TATCTTAGTTTTTGTTGGATCATGTTGAGTCTGTCAGCTCCACAGGACTT
CAGTACGTTTCTGAACAGTCCCTGCCATCTCTACGGGGGAGAGGGTCAGG
CAAGCTGCAAGTGACACTCACCTCCTGCTGACAGTTGCAGTGTCTCAGAT
GGCCTGGAAGGGTGGTCTCCAGCAGCCTGCTGGGCGCTCCCCTTTCATGA
GAGCCACCTGCAGTGACCTGAACTGATACATGTTGATTAGTCTGCCCTTT
CTTTAGAAAACTGCTACTCTCCTTTTCATATCTCAGAAAAACAGTAGAGG
CCTTTTAGGACCAAACTCCATGTCACACTGATGAAGAGCCAGTGGGGGTT
AGAGCGTCCTGTTAAGGCACATGCTAGCTTCCCACTCAAGTCTGGCAGCG
CTGGGGCATCAGCACACCTCTTGCCACCCACACTGATACCAGAGGGGAAG
GCTGTGAGGTGGCTGGGGGTTGAGACTTGAGGTTTCTAACTTTCCTCTGC
ACACCTGTGGCTACCTGGTGTTTGTCTCTTGATTCCCTCCACCTGCCTCA
CACCCTGCCTCCGTCGGGATTTTCCACCTACACCATTCAAAAGGAACATA
GGAGAGGGCATGAAGGGGCTAGGCTGAAGCACTCTGATGACTGGGGCCAA
TTTGTGGCTGAAAATGAATACATTTTTTGAAATTTATGGTCATTTTCAAG
TGATTTAGAAGGTTGATCCTTAGCCTCATACAGTGATGAAATAATCTGTG
TGTTCAGAGCCAAGCAGGACTTTAGCAAGAGTCTGATTGTATTGTCACTA
TCTCGGGGAAAAAAAAATACAAATACATTTCTCTGATCTCTGATGGCAAT
GAAGTTTGACTTGTAAAAAAAAAAAAAAAAAAAAAAAAAAAAAAAAAAAA
AAAAAAAAA
Human GOSR2 mRNA sequence - var3
(public gi: 16307240)

(SEQ ID NO: 65)

CCCACGCGTCCGGTTCCGAGGAAGCCAGAGCCGGAGCCGTGGCCTGCCGG
GCCGGCGACATGGATCCCCTGTTCCAGCAAACGCACAAGCAGGTCCACGA
GATCCAGTCTTGCATGGGACGCCTGGAGACGGCAGACAAGCAGTCTGTGC
ACATAGTAGAAAACGAAATCCAAGCAAGCATAGACCAGATATTCAGCCGT
CTAGAACGTCTGGAGATTTTGTCCAGCAAGGAGCCCCCTAACAAAAGGCA
AAATGCCAGACTTCGGGTTGACCAGTTAAAGTATGATGTCCAGCACCTGC
AGACTGCGCTCAGAAACTTCCAGCATCGGCGCCATGCAAGGGAGCAGCAG
GAGAGACAGCGAGAAGAGCTTCTGTCTCGAACCTTCACCACTAACCACTC
TGACACCACCATACCAATGGACGAATCACTGCAGTTTAACTCCTCCCTCC
AGAAAGTTCACAACGGCATGGATGACCTCATTTTAGATGGGCACAATATT
TTAGATGGACTGAGGACCCAGAGACTGACCTTGAAGGGGACTCAGAAGAA
GATCCTTGACATTGCCAACATGCTGGGCTTGTCCAACACAGTGATGCGGC
TCATCGAGAAGCGGGCTTTCCAGGACAAGTACTTTATGATAGGTGGGATG
CTGCTGACCTGTGTCGTCATGTTCCTCGTGGTGCAGTACCTGACATGAGC
CAGCCACGCTCAGTGGCTGAACAGCATTCCCACAGCCTGCAAGTGTGTGT
GTGTGTGAAAGAGAGAGGGGGGCCCAGAGGCCGCCTTTTGAAATGTTTGC
CTGTCTGAACTGTGAAGACACTTGGGAGTGATTGTGGTCTAATTTCCAAA
AAAAAAAAAAAAAAAAAAAAAAAAAAAAAAAAAAAA
Human GOSR2 mRNA sequence - var4
(public gi: 16905519)

(SEQ ID NO: 66)

GTTCCGAGGAAGCCAGAGCCGGAGCCGTGGCCTGCCGGGCCGGCGACATG
GATCCCCTGTTCCAGCAAACGCACAAGCAGGTCCACGAGATCCAGTCTTG
CATGGGACGCCTGGAGACGGCAGACAAGCAGTCTGTGCACATAGTAGAAA
ACGAAATCCAAGCAAGCATAGACCAGATATTCAGCCGTCTAGAACGTCTG
GAGATTTTGTCCAGCAAGGAGCCCCCTAACAAAAGGCAAAATGCCAGACT
TCGGGTTGACCAGTTAAAGTATGATGTCCAGCACCTGCAGACTGCGCTCA
GAAACTTCCAGCATCGGCGCCATGCAAGGGAGCAGCAGGAGAGACAGCGA
GAAGAGCTTCTGTCTCGAACCTTCACCACTAACGACTCTGACACCACCAT
ACCAATGGACGAATCACTGCAGTTTAACTCCTCCCTCCAGAAAGTTCACA
ACGGCATGGATGACCTCATTTTAGATGGGCACAATATTTTAGATGGACTC
AGGACCCAGAGACTGACCTTGAAGGGGACTCAGAAGAAGATCCTTGACAT
TGCCAACATGCTGGGCTTCTCCAACACAGTGATCCCGCTCATCGAGAAGC
GGGCTTTCCAGGACAAGTACTTTATGATAGGCACCCAACGATCCTGCCAG
ACAGCACACTTTGGAGGAAGGTCTGCAGGGAGCAGCTGAGCCATTTGTTC
TTGAACTCTGGGAGGCAGAAGTCCCCGCACCCATCATGCGTGGACTGATA
GGACATCTTTTCGTGGTGTGCACCAGTGCTTTCCACACTTGACAGTGGTT
GGCTTTGATGAACCCTCATGCTGCACCTTCAGAGCCAGTCCTCTAGTTTG
GAATAAAAATTGCAGAGGTGGAAAAAAAAAAAAAAAAAAA
Human GOSR2 mRNA sequence - var5
(public gi: 12711466)

(SEQ ID NO: 67)

AGCCGGAGCCGTGGCCTGCGGGGCCGGCGACATCGATCCCCTGTTCCAGC
AAACGCACAAGCAGGTCCACGAGATCCAGTCTTGCATGGGACGCCTGGAG
ACGGCAGACAAGCAGTCTGTGCACATAGTAGAAAACGAAATCCAAGCAAG
CATAGACCAGATATTCAGCCGTCTAGAACGTCTGGAGATTTTGTCCAGCA
AGGAGCCCCCTAACAAAAGGCAAAATGCCAGACTTCCGGTTGACCAGTTA
AAGTATGATGTCCAGCACCTGCAGACTGCGCTCAGAAACTTCCAGCATCG
GCGCCATGCAAGGGAGCAGCAGGAGAGACAGCGAGAAGAGCTTCTGTCTC
GAACCTTCACCACTAACGACTCTGACACCACCATACCAATCGACGAATCA
CTGCAGTTTAACTCCTCCCTCCACAAAGTTCACAACGGCATGGATGACCT
CATTTTAGATCGGCACAATATTTTAGATGGACTGAGGACCCAGAGACTGA
CCTTGAAGGGGACTCAGAAGAAGATCCTTGACATTGCCAACATGCTGGGC
TTGTCCAACACAGTGATGCGGCTCATCGAGAAGCGGGCTTTCCAGGACAA
GTACTTTATGATAGGCACCCAAGGATCCTGCCAGACAGCACACTTTGGAG
GAAGGTCTGCAGGGAGCAGCTGAGCCATTTGTTCTTGAACTCTGCGAGGC
AGAAGTCCCCGCACCCATCATGCGTGGACTGATAGGACATCTTTTCGTGG
TGTGCACCAGTGCTTTCCACACTTGACAGTGGTTGGCTTTGATGAACCCT
CATGCTGCACCTTCAGAGCCAGTCCTCTAGTTTGGAATAAAAATTGCAGA
GGTGGAAAAAAAAAAAAAAAAAAA
Human GOSR2 mRNA sequence - var6
(public gi: 16905521)

(SEQ ID NO: 68)

GTTCCGAGGAACCCAGAGCCGGAGCCGTGGCCTGCCGGGCCGGCGACATG
GATCCCCTGTTCCAGCAAACGCACAAGCAGGTCCACGAGATCCAGTCTTG
CATGGGACGCCTGGAGACGGCAGACAAGCAGTCTGTGCACATAGTAGAAA
ACGAAATCCAAGCAAGCATAGACCAGATATTCAGCCGTCTAGAACGTCTG
GAGATTTTGTCCAGCAAGGAGCCCCCTAACAAAAGGCAAAATGCCAGACT
TCGGGTTGACCAGTTAAAGTATGATGTCCAGCACCTGCAGACTGCGCTCA
GAAACTTCCAGCATCGGCGCCATGCAAGGGAGCAGCAGGAGAGACAGCGA
GAAGAGCTTCTGTCTCGAACCTTCACCACTAACGACTCTGACACCACCAT
ACCAATGGACGAATCACTGCAGTTTAACTCCTCCCTCCAGAAAGTTCACA
ACGGCATGGATGACCTCATTTTAGATGGGCACAATATTTTAGATGGACTG
AGGACCCAGAGACTGACCTTGAAGGGGACTCAGAAGAAGATCCTTGACAT
TGCCAACATGCTGGGCTTGTCCAACACAGTGATGCGGCTCATCGAGAAGC
GGGCTTTCCAGGACAAGTACTTTATGATAGGTGGGATGCTGCTGACCTGT
GTGGTCATGTTCCTCGTGGTGCAGTACCTGACATGAGCCAGCCACGCTCA
GTGGCTGAACAGCATTCCCACAGCCTGCAAGTGTGTGTGTGTGTGAAAGA
GAGAGGGGGGCCCAGAGGCCGCCTTTTGAAATGTTTGCCTGTCTGAACTG
TGAAGACACTTGGGAGTGATTGTGGTCTAATTTCCAAAAAAAAAAAAAAA
AAAAAAAAAAAAAAAAAAAAAAAA
Human GOSR2 Protein sequence - var1
(public gi: 2316088)

(SEQ ID NO: 89)

MDPLFQQTNKQVHEIQSCMGRLETADKQSVHIVENEIQASIDQIFSRLER
LEILSSKEPPNKRQNARLRVDQLKYDVQHLQTALRNFQHRRHAREQQERQ
RSELLCRTFTTNGSDTTIPMDESLQFNSSLQKVHNGMDDLILDGHNILDG
LRTQRLTLKGTQKKIPDIANMLGLSNTVMRLIEKRAFQDKYFMIGGMLLT
CVVMFLVVQYLT
Human GOSR2 Protein sequence - var2
(public gi: 21961349)

(SEQ ID NO: 90)

MDPLFQQTHKQVHEIQSCMGRLETADKQSVHIVENEIQASIDQIFSRLER
LEILSSKEPPNKRQNARLRVDQLKYDVQHLQTALRNFQHRRHAREQQERQ
REELLSRTFTTNDSDTTIPMDESLQFNSSLQKVHNGMDDLILDGHNILDG
LRTQRLTLKVGSLLGDREKASCFSLIQQFSNCVYILITCPQIVIF
Human GOSR2 Protein sequence - var3
(public gi: 12711467)

(SEQ ID NO: 91)

MDPLFQQTHKQVHEIQSCMGRLETADKQSVHIVENEIQASIDQIFSRLER
LEILSSKEPPNKRQNARLRVDQLKYDVQHLQTALRNFQHRRHAREQQERQ
REELLSRTFTTNDSDTTIPMDESLQFNSSLQKVHNGMDDLILDGHNILDG
LRTQRLTLKGTQKKILDIANMLGLSNTVMRLIEKRAFQDKYFMIGTQGSC
QTAHFGGRSAGSS
Human GOSR2 Protein sequence - var4
(public gi: 16905522)

(SEQ ID NO: 92)

MDPLFQQTHKQVHEIQSCMGRLETADKQSVHIVENEIQASIDQIFSRLER
LEILSSKEPPNKRQNARLRVDQLKYDVQHLQTALRNFQHRRHAREQQERQ
REELLSRTFTTNDSDTTIPMDESLQFNSSLQKVHMGMDDLILDGHNILDG
LRTQRLTLKGTQKKILDIANMLGLSNTVMRLIEKRAFQDKYFMIGGMLLT
CVVMFLVVQYLT
Human RALA mRNA sequence - var1
(public gi: 35845)

(SEQ ID NO: 69)

ATGGCTGCAAATAAGCCCAAGGGTCAGAATTCTTTGGCTTTACACAAAGT
CATCATGGTGGGCAGTGGTGGCGTGGGCAACTCAGCTCTGACTCTACAGT
TCATCTACGATGAGTTTGTGGAGGACTATGAGCCTACCAAAGCAGACAGC
TATCGGAAGAAGGTAGTGCTAGATGGGGAGGAAGTCCAGATCGATATCTT
AGATACAGCTGCGCAGGAGGACTACGCTGCAATTAGAGACAACTACTTCC
GAAGTGGGGAGGGGTTCCTCTGTGTTTTCTCTATTACAGAAATGGAATCC
TTTGCAGCTACACCTGACTTCAGGGAGCAGATTTTAAGAGTAAAAGAAGA
TGAGAATGTTCCATTTCTACTGGTTGGTAACAAATCAGATTTAGAAGATA
AAAGACAGGTTTCTGTAGAAGAGGCAAAAAACACAGCTGAGCAGTGGAAT
GTTAACTACGTGGAAACATCTGCTAAAACACGAGCTAATGTTGACAAGGT
ATTTTTTGATTTAATGAGAGAAATTCGAGCGAGAAAGATGGAAGACAGCA
AAGAAAAGAATCGAAAAAAGAAGAGGAAAAGTTTAGCCAAGAGAATCAGA
GAAAGATGCTGCATTTTATAA
Human RALA mRNA sequence - var2
(public gi: 24980846)

(SEQ ID NO: 70)

CCGCTCCCCAGAGCAAAGCGTCCGACTCCTCCTCCTCCTTCTCCTCCTCC
TCCTCCTCCTCCTCCAGCCGCCCAGGCTCCCCCGCCACCCGTCAGACTCC
TCCTTCGACCGCTCCCGGCGCGGGGCCTTCCAGGCGACAAGGACCGAGTA
CCCTCCGGCCGGAGCCACGCAGCCGCGGCTTCCGGAGCCCTCGGGGCGGC
GGACTGGCTCGCGGTGCAGATTCTTCTTAATCCTTTGGTGAAAACTGAGA
CACAAAATGGCTGCAAATAAGCCCAAGGGTCAGAATTCTTTGGCTTTACA
CAAAGTCATCATGGTGGGCAGTGGTCGCCTGGGCAAGTCAGCTCTGACTC
TACAGTTCATGTACGATGAGTTTGTGGAGGACTATGAGCCTACCAAAGCA
GACAGCTATCGGAAGAAGGTAGTGCTAGATGGGGAGGAAGTCCAGATCGA
TATCTTAGATACAGCTGGGCAGGAGGACTACGCTGCAATTAGAGACAACT
ACTTCCGAAGTGGGGACGGGTTCCTCTGTCTTTTCTTATTACAGAAATGC
AAATCCTTTGCAGCTACAGCTGACTTCAGGGAGCAGATTTTAAGAGTAAA
AGAAGATGAGAATGTTCCATTTCTACTGGTTGGTAACAAATCAGATTTAG
AAGATAAAAGACAGGTTTCTGTAGAAGAGGCAAAAAACAGAGCTGAGCAG
TGGAATGTTAACTACGTGGAAACATCTGCTAAAACACGAGCTAATGTTGA
CAAGGTATTTTTTCATTTAATGAGAGAAATTCGAGCGAGAAAGATGGAAG
ACAGCAAAGAAAAGAATGGAAAAAAGAAGAGGAAAAGTTTAGCCAAGAGA
ATCAGAGAAAGATCCTGCATTTTATAATCAAAGCCCAAACTCCTTTCTTA
TCTTGACCATACTAATAAATATAATTTATAAGCATTGCCATTGAAGGCTT
AATTGACTGAAATTACTTTAACATTTTGGAAATTGTTGTATATCACTAAA
AGCATGAATTGGAACTGCAATGAAAGTCAAATTTACTTTAAAAAGAAATT
AATATGGCTTCACCAAGAAGCAAAGTTCAACTTATTTCATAATTGCCTAC
ATTTATCATGGTCCTGAATGTAGCGTGTAAGCTTGTGTTTCTTGGGCAGT
CTTTCTTGAAATTGAAGAGGTGAAATGGGGGTCGGGAGTGGGAGGAAAGG
TGACTTCCTCTGGTGTTTATTATAAAGCTTAAATTTTATATCATTTTAAA
ATGTCTTGGTCTTCTACTGCCTTGAAAAATGACAATTGTGAACATGATAG
TTAAACTACCACTTTTTTTAACCATTATTATGCAAAATTTAGAAGAAAAG
TTATTGGCATGGTTGTTGCATATACTTAAACTGAGAGTAATTCATCTGTG
AATCTGCTTTAATTACCTGGTGAGTAACTTAGAAAACTGGTGTAAACTTG
TACATGGAATTTTTTGAATATGCCTTAATTTAGAAACTGAAAAATATCTG
GTTATATCATTCTGGGTGTGTTCTTACTGACACCAGGGGTCCGCTGCCCC
ATGTGTCCTGGTGAGAAAATATATGCCTGGCACAGCTTTTGTATAGAAAA
TTCTTGAGAAGTAACTGTCCGCTAGAAGTCTGTCCAAATTTAAAATGTGT
GCCATATTCTGGTTCTTGAAAATAAGATTCCAGAGCTCTTTGATCGCTTT
TAATAAACTGCAAGTTCATTTTAAATGAAGGGCCAGCATATATACTTGCA
AGATAATTTTCAGCTGCAAGGATTCAGCACCAGTTATGTTTGAATGAACC
CTCCTTTTCTCTGAGATTCTGGTCCCTGGAAATCCCTTTCTGCTAGTGGT
GACCATGTAAGTGTTAAGTTTTTAATCTCGGAGCAGGGCATAGGAAGAAA
ATGTCAGTAGTGCTAATCCATTTTGCACTAGAACGCTTCGGGAAAATATT
CATGCTTGCCATCTGTTCATTTCTAAATTTATATTCATAAAGTTACAGTT
TGATACAGGAATTATTAGGAGTAATTCTTTTCTGTTTCTGTTTATAATGA
AGAACACTGTAGCTACATTTTCAGAAGTTAACATCAACCCATCAAACCTG
GGTATAGTGCAGAAAACGTGGCACACACTGACCACACATTAGGCTGTGTC
ACCATTGTGTGGTGTACCTGCTGGAAGAATTCTAGCATGCTACTTCGGGA
CATAATTTCAGTGGGAAATATGCCACTGACCGATTTTTTTTTTTTCCTCT
TTGCAGTGGGGCTAGGACAGTTGATTCAACAAAGTATTTTTTTCTTTTTT
CTCAGTCCTAATTTGAACAGGTCAAAGATGTGTTCAGGCATTCCAGGTAA
CAGGTGTGTATGTAAAGTTAAAAATAGGCTTTTTAGGAACTCACTCTTTA
GATATTTACATCCACCTTCTCATGTTAAATATTTGTCCTTAAAGGGTTTG
AGATGTACATCTTTCATTTCGTATTTCTCATAGGCTATGCCATGTGCGGA
ATTCAAGTTACCAATGTAACACTGGCCAGCGGGCCCAGCAATCTCCATGT
GTACTTATTACAGTCTTATTTAACCAGGGGTCCTAACCACTAACATTGTG
ACTTTGCTTTGAGACCTTTCCTCTCCTGGGTACTGAGGTGCTATGAACCC
AACTGACAAAGATGCATCACGTGTCTTAGGCTGATGCCACTACCCGATTT
GTTTATTTGCAATTTGAGCCATTTAAAGACCAATAAACTTCCTTTTTTAA
AAAAAAAAAAAAAAAAAAAAAAAA
Human RALA mRNA sequence - var3
(public gi: 3483427)

(SEQ ID NO: 71)

ATAATCAAAGCCCAAACTCCTTTCTTATCTTGACCATACTAATAAATATA
ATTTATAAGCATTGCCATTGAAGGCTTAATTGACTGAAATTACTTTAACA
TTTTGGAAATTGTTGTATATCACTAAAAGCATGAATTGGAACTGCAATGA
AAGTCAAATTTACTTTAAAAAGAAATTAATATGGCTTCACCAAGAAGCAA
AGTTCAACTTATTTCATAATTGCCTACATTTATCATGGTCCTGAATGTAG
CGTGTAACCTTGTGTTTCTTGGGCAGTCTTTCTTGAAATTGAAGAGGTGA
AATGGGGGTGGGGAGTGGGAGGAAAGGTGACTTCCTCTGGTGTTTATTAT
AAAGCTTAAATTTTATATCATTTTAAAATGTCTTGGTCTTCTACTGCCTT
GAAAAATGACAATTGTGAACATGAThGTTAAACTACCACTTTTTTTAACC
ATTATTATGCAAAAAAAAAA
Human RALA mRNA sequence - var4
(public gi: 20147712)

(SEQ ID NO: 72)

ATGGTCGACTACCTAGCAAATAAGCCCAAGGGTCAGAATTCTTTGGCTTT
ACACAAAGTCATCATGGTGGGCAGTGGTGGCGTGGGCAAGTCAGCTCTGA
CTCTACAGTTCATGTACGATGAGTTTGTGGAGGACTATGAGCCTACCAAA
GCAGACAGCTATCGGAAGAAGGTAGTGCTAGATGGGGAGGAAGTCCAGAT
CGATATCTTAGATACAGCTGGGCAGGAGGACTACGCTGCAATTAGAGACA
ACTACTTCCGAAGTGGGGAGGGGTTCCTCTGTGTTTTCTCTATTACAGAA
ATGGAATCCTTTGCAGCTACAGCTGACTTCAGGGAGCAGATTTTAAGAGT
AAAAGAAGATGAGAATGTTCCATTTCTACTGGTTGGTAACAAATCAGATT
TAGAAGATAAAAGACAGGTTTCTGTAGAAGAGGCAAAAAACAGACCTGAG
CAGTGGAATGTTAACTACGTGGAAACATCTGCTAAAACACGAGCTAATGT
TGACAAGGTATTTTTTGATTTAATGAGAGAAATTCGAGCGAGAAAGATCG
AAGACAGCAAAGAAAAGAATGGAAAAAAGAAGAGGAAAAGTTTAGCCAAG
AGAATCAGAGAAAGATGCTGCATTTTATAA
Human RALA mRNA sequence - var5
(public gi: 10439805)

(SEQ ID NO: 73)

AGAATGGAAAAAAGAAGAGGAAAAGTTTAGCCAACAGAATCAGAGAAAGA
TGCTGCATTTTATAATCAAAGCCCAAACTCCTTTCTTATCTTGACCATAC
TAATAAATATAATTTATAAGCATTGCCATTCAAGGCTTAATTGACTGAAA
TTACTTTAACATTTTGGAAATTGTTGTATATCACTAAAAGCATGAATTCG
AACTGCAATGAAAGTCAAATTTACTTTAAAAAGAAATTAATATGGCTTCA
CCAAGAAAGCAACTTCAACTTATTTCATAATTGCCTACATTTATCATGGT
CCTGAATCTAGCGTGTAAGCTTGTGTTTCTTGGGCAGTCTTTCTTGAAAT
TGAAGAGGTGAAATGGGGGTGGGGAGTGCGAGGAAAGGTGACTTCCTCTG
GTGTTTATTATAAAGCTTAAATTTTATATCATTTTAAAATGTCTTGGTCT
TCTACTGCCTTGAAAAATGACAATTGTGAACATGATAGTTAAACTACCAC
TTTTTTTAACCATTATTATCCAAAATTTAGAAGAAAACTTATTGGCATGG
TTGTTGCATATAGTTAAACTGAGAGTAATTCATCTCTGAATCTGCTTTAA
TTACCTGGTGAGTAACTTAGAAAAGTGGTGTAAACTTGTACATGGAATTT
TTTGAATATGCCTTAATTTAGAAACTGAAAAATATCCGGTTATATCATTC
TGGGTGTGTTCTTACTGACACCAGGGGTCCGCTGCCCCATGTGTCCTGGT
GAGAAAATATATGCCTGGCACAGCTTTTGTATAGAAAATTCTTGAGAAGT
AACTGTCCGCTAGAAGTCTGTCCAAATTTAAAATGTGTGCCATATTCTGG
TTCTTGAAAATAAGATTCCAGAGCTCTTTGATCGCTTTTAATAAACTGCA
AGTTCATTTTAATTGAAGGGCCAGCATATATACTTGCAAGATAATTTTCA
GCTGCAACGATTCAGCACCAGTTATGTTTGAATGAACCCTCCTTTTCTCT
GAGATTCTGGTCCCTCGAAATCCCTTTCTGCTAGTGGTGAGCATGTAAGT
GTTAAGTTTTTAATCTGGGAGCAGGGCATAGGAAGAAAATGTCAGTAGTG
CTAATGCATTTTGCACTAGAACGCTTCGGGAAAATATTCATGCTTGCCAT
CTGTTCATTTCTAAATTTATATTCATAAAGTTACAGTTTGATACAGGAAT
TATTAGGAGTAATTCTTTTCTGTTTCTGTTTATAATGAAGAACACTGTAG
CTACATTTTCAGAAGTTAACATCAAGCCATCAAACCTGGGTATAGTGCAG
AAGACGTGGCACACACTGACCACACATTAGGCTGTGTCACCATTGTGTGG
TGTACCTGCTGGAAGAATTCTAGCATGCTACTTGGGGACATAATTTCAGT
GGGAAATATGCCACTGACCGATTTTTTTTTTTTTCCTCTTTGCAGTGGGG
CTAGGACAGTTGATTCAACAAAGTATTTTTTTCTTTTTTCTCAGTCCTAA
TTTGGACAGGTCAAAGATGTGTTCAGGCATTCCAGGTAACAGGTGTGTAT
GTAAAGTTAAAAATAGGCTTTTTAGGAACTCACTCTTTAGATATTTACAT
CCAGCTTCTCATGTTAAATATTTGTCCTTAAAGGGTTTGAGATGTACATC
TTTCATTTCGTATTTCTCATAGGCTATGCCATGTGCGGAATTCAAGTTAC
CAATGTAACACTGGCCAGCGGGCCCAGCAATCTCCATGTGTACTTATTAC
AGTCTTATTTAACCAGGGGTCCTAACCACTAACATTGTGACTTTGCTTTG
AGACCTTTCCTCTCCTGGGTACTGAGGTGCTATGAAGCCAACTGACAAAG
ATGCATCACGTGTCTTAGGCTGATGCCACTACCCGATTTGTTTATTTGCA
TTTGAGCCATTTAAAGACCAATAAACTTCCTTTTTTAAAAAAAAAAAAAA
AAAAAAAAAAAAAAA
Human RALA Protein sequence - var1
(public gi: 35846)

(SEQ ID NO: 93)

MAANKPKGQNSLALMKVIMVGSGGVGKSALTLQFMYDEFVEDYEPTKADS
YRKKVVLDGEEVQIDILDTAGQEDYAAIRDNYFRSGEGFLCVFSITEMES
FAATADFREQILRVKEDENVPFLLVGNKSDLEDKRQVSVEEAKNRAEQWN
VNYVETSAKTRANVDKVFFDLMREIRARKMEDSKEKNGKKKRKSLAKRIR
ERCCIL
Human RALA Protein sequence - var2
(public gi: 20147713)

(SEQ ID NO: 94)

MVDYLANKPKCQNSLALHKVIMVGSGGVGKSALTLQFMYDEFVEDYEPTK
ADSYRKKVVLDGEEVQIDILDTAGQEDYAAIRDNYFRSGEGFLCVFSITE
MESFAATADFREQILRVKEDENVPFLLVGNKSDLEDKRQVSVEEAKNRAE
QWNVNYVETSAKTRANVDKVFFDLMREIRARKMEDSKEKNGKKKRKSLAK
RIRERCCIL
Below are sequences representative of the POSH-
pathway polypeptide, Dynamin II:
Human Dynamin II mRNA sequence
(public gi: 1196422)

(SEQ ID NO: 74)

GACCGTGAGGCCGAGCCGGGAGCGGGCGTCTTGCCGAGGCCCGGGCGGGC
GGGAGCAACGGCTACAGACGCCGCGGGGCCAGGTCGTTGAGGGTCGGCGG
CGGGCGAGGAGCGCAGGGCGCTCGGGCCGGGGGCCGCCGGCGCCATGGGC
AACCGCGGGATGGAAGAGCTGATCCCGCTGGTCAACAAACTGCAGGACGC
CTTCAGCTCCATCGGCCAGAGCTGCCACCTGGACCTGCCGCACATCGCTG
TAGTGGGCGGCCAGAGCGCCGGCAAGAGCTCGGTGCTGGAGAACTTCGTG
GGCCGGGACTTCCTTCCCCGCGGTTCAGGAATCGTCACCCGGCGGCCTCT
CATTCTGCAGCTCATCTTCTCAAAAACAGAACATGCCGAGTTTTTGCACT
GCAAGTCCAAAAAGTTTACAGACTTTGATGAAGTCCGGCACGAGATTGAA
GCAGAGACCGACAGGGTCACGGGGACCAACAAAGGCATCTCCCCAGTGCC
CATCAACCTTCGAGTCTACTCGCCACACGTGTTGAACTTGACCCTCATCG
ACCTCCCGGGTATCACCAAGGTGCCTGTGGGCGACCAGCCTCCAGACATC
GAGTACCGAGTCAAGGACATGATCCTGCAGTTCATCAGCCGGGAGAGCAG
CCTCATTCTGGCTGTCACGCCCGCCAACATCGACCTGGCCAACTCCGACG
CCCTCAACCTGGCCAAGGAAGTCGATCCCCAAGGCCTACGGACCATCGGT
CTCATCACCAAGCTTGACCTGATGGACGAGGGCACCGACGCCAGGGACGT
CTTGGAGAACAAGTTGCTCCCGTTGAGAAGAGGCTACATTGCCGTGGTGA
ACCGCAGCCAGAAGGATATTGAGGGCAAGAAGGACATCCGTGCAGCACTG
GCAGCTGAGAGGAAGTTCTTCCTCTCCCACCCGGCCTACCGGCACATGGC
CGACCGCATGGGCACGCCACATCTGCAGAAGACCCTGAATCAGCAACTGA
CCAACCACATCCGGGAGTCGCTGCCGGCCCTACGTAGCAAACTACAGAGC
CAGCTCCTGTCCCTGGAGAACGAGGTGGAGGAGTACAAGATCTTTCGGCC
CGACGACCCCACCCCTAAAACCAAAGCCCTGCTGCAGATGGTCCAGCAGT
TTGGGGTGGATTTTGAGAAGAGGATCGAGGGCTCAGGAGATCAGGTGGAC
ACTCTGGAGCTCTCCGGGGGCGCCCGAATCAATCGCATCTTCCACGAGCG
GTTCCCATTTGAGCTGGTGAAGATGGAGTTTGACGAGAAGGACTTACGAC
GGGAGATCAGCTATGCCATTAAGAACATCCATGGAGTCAGGACCGGGCTT
TTCACCCCGGACTTGGCATTCGAGGCCATTGTGAAAAAGCAGGTCGTCAA
GCTGAAAGAGCCCTGTCTGAAATGTGTCGACCTGGTTATCCAGGAGCTAA
TCAATACAGTTAGGCAGTGTACCAGTAAGCTCAGTTCCTACCCCCGGTTG
CGAGAGGACACAGAGCGAATCGTCACCACTTACATCCCGGAACGGGAGGG
GAGAACGAAGGACCAGATTCTTCTGCTGATCGACATTGAGCAGTCCTACA
TCAACACCAACCATGAGGACTTCATCGGGTTTGCCAATGCCCAGCAGAGG
ACCACGCAGCTGAACAAGAAGAGAGCCATCCCCAATCAGGTGATCCGCAG
GGGCTGGCTGACCATCAACAACATCAGCCTGATGAAAGGCGGCTCCAAGG
AGTACTGGTTTGTGCTGACTGCCGAGTCACTGTCCTGGTACAAGGATGAG
GAGGAGAAAGAGAAGAAGTACATGCTGCCTCTGGACAACCTCAAGATCCG
TGATGTGGAGAAGGGCTTCATGTCCAACAAGCACGTCTTCGCCATCTTCA
ACACGGAGCAGAGAAACGTCTACAAGGACCTGCGGCAGATCGAGCTGGCC
TGTGACTCCCAGGAAGACGTGGACAGCTGGAAGGCCTCGTTCCTCCGACC
TGGCGTCTACCCCGAGAACGACCAGGCAGAAAACGAGGATGGGGCCCAGG
AGAACACCTTCTCCATGGACCCCCAACTGGACCGGCAGGTGGAGACCATT
CGCAACCTGGTGGACTCATACGTGGCCATCATCAACAAGTCCATCCGCGA
CCTCATGCCAAAGACCATCATGCACCTCATGATCAACAATACGAAGGCCT
TCATCCACCACGAGCTGCTGGCCTACCTATACTCCTCGGCAGACCACACC
AGCCTCATGGACGAGTCGGCTGACCAGGCACAGCGGCGGGACGACATGCT
GCGCATGTACCATGCCCTCAAGGAGGCGCTCAACATCATCGGTGACATCA
GCACCAGCACTGTGTCCACGCCTGTACCCCCGCCTGTCGATGACACCTGG
CTCCAGACCGCCAGCAGCCACAGCCCCACTCCACAGCGCCGACCGGTGTC
CAGCATACACCCCCCTGGCCGGCCCCCAGCAGTGAGGGGCCCCACTCCAG
GGCCCCCCCTGATTCCTGTTCCCGTGGGGGCAGCAGCCTCCTTCTCCGCG
CCCCCAATCCCATCCCGGCCTGGACCCCAGAGCGTGTTTGCCAACAGTGA
CCTCTTCCCAGCCCCGCCTCAGATCCCATCTCGGCCAGTTCGGATCCCCC
CAGGGATTCCCCCAGGAGTGCCCAGCAGAAGACCCCCTGCTGCGCCCAGC
CCGCCCACCATTATCCGCCCAGCCGAGCCATCCCTGCTCGACTAGGCCTC
GACGGGGGCGTGCTCTCGGGGGGGCCTCACGCACCCGCGGCGCAGGAGCT
TCAGTGGTCTGGGGCCCTCCGCCGCCCCTATGCTCGGACCAGGCTCCCAG
TGGGCAGCCCTGGCCTCTTGGTTAACGCTGGCCCCGGTCCAGGGCCGGCC
CCTGTGCCTGGCTGGACACCGCACTGCGCAAAGGGGCCCTGGAGCTCCAG
GCAGGGGGCGCTGGGGTGTTGCACTTTGGGGGATGGAGTCTCAGGGTGGC
AGAGGGGGGACCAGAACCCTTGACACCATCCTGAATGAGGGGTCCAGCCT
GGGGGGGACTCTACCAAGGTCTTCTTGGGCTGGGAAAGCCCATGTAGGGC
AGGCCTTCTATAAGTGCGGGCACCAAGGGCGCCTACATCCCCAGGCCTTG
CTGGGGTGCAGGGGTATATCAACTTCCCATTAGCAGGAGCTCCCCAGCGG
CAAGCCTGGCCCAGTGGGCTCGGTAGTGCCCAGCTGGCAGGCCTGAGGTG
TACATAGTCCTTCCCGGCCATATTAACCACACAGCCTGAGCCTGGCCCAG
CCTCGGCTGCCAGAGGTGCCTTTGCTAGGCCCGGAGCCGTTGCCCGGCCT
TGCCCTTGCCCTATTCCTCTCCTCCTCCTCCTCCTGGGTCCCCCAGGGTG
GCTGGGCTTGGGCTATGTGGGTGGTGGTGGCGGGGGGTCTTGGGGGCCTC
TCAGCTCCCGCCCATGCCTCCCTGATGGGTGGGCCCAGGGCGGCCTCTCT
CTGAGGAGACCTCACCCACTCCTCGCTCAGTTTGACCACTGTAAGTGCCT
GCACTCTGTATTCTATTAATAAACTAAAATAAAGGGAAGAGCGTGCTGGT
G
Human Dynamin II Protein sequence - var1
(public gi: 1706539)

(SEQ ID NO: 95)

MGNRGMEELIPLVNKLQDAFSSIGQSCHLDLPQIAVVGGQSAGKSSVLEN
PVGRDFLPRGSGIVTRRPLILQLIFSKTEHAEFLHCKSKKFTDFDEVRQE
IEAETDRVTGTNKGISPVPINLRVYSPHVLNLTLIDLPGITKVPVGDQPP
DIEYRVKDMILQFISRESSLILAVTPANMDLANSDALKLAKEVDPQGLRT
IGVITKLDLMDEGTDARDVLENKLLPLRRGYIGVVNRSQKDIEGKKDIRA
ALAAERKFFLSHPAYRHMADRNGTPHLQKTLNQQLTNHIRESLPALRSKL
QSQLLSLEKEVEEYKIFRPDDPTPKTKALLQMVQQFGVDFEKRIECSGDQ
VDTLELSGGARINRIFHERFPFELVKMEFDEKDLRREISYAIKNIHGVRT
GLFTPDLAFEAIVKKQVVKLKEPCLKCVDLVIOELINTVRQCTSKLSSYP
RLREETERIVTTYIREREGRTKDQILLLIDIEQSYINTNHEDFIGFANAQ
QRSTQLNKKRAIPNQGEILVIRRGWLTINNISLMKGGSKEYWFVLTAESL
SWYKDEEEKEKKYMLPLDNLKIRDVEKGFMSNKHVFAIFNTEQRNVYKDL
RQIELACDSQEDVDSWKASFLRAGVYPEKDQAENEDGAQENTFSMDPQLE
RQVETIRNLVDSYVAIINKSIRDLMPKTIMHLMINNTKAFIHMELLAYLY
SSADQSSLMEESADQAQRRDDMLRMYHALKEALNIIGDISTSTVSTPVPP
PVDDTWLQSASSHSPTPQRRPVSSIHPPGRPPAVRGPTPGPPLIPVPVGA
AASFSAPPIPSRPGPQSVFANSDLFPAPPQIPSRPVRIPPGIPPGVPSRR
PPAAPSRPTIIRPAEPSLLD
Human Dynamin II Protein sequence - var2
(public gi: 1196423)

(SEQ ID NO: 96)

MGNRGMEELIPLVNKLQDAFSSIGQSCHLDLPQIAVVGGQSAGKSSVLEN
FVGRDFLPRGSGIVTRRPLILQLIFSKTEHAEFLHCKSKKFTDFDEVRQE
IEAETDRVTGTNKGISPVPINLRVYSPHVLNLTLIDLPGITKVPVGDQPP
DIEYRVKDMILQFISRESSLILAVTPANMDLANSDALKLAKEVDPQGLRT
IGVITKLDLMDEGTDARDVLENKLLPLRRGYIGVVNRSQKDIEGKKDIRA
ALAAERKFFLSHPAYRIMADRMGTPHLQKTLNQQLTNHIRESLPALRSKL
QSQLLSLEKEVEEYKIFRPDDPTPKTKALLQMVQQFGVDFEKRIEGSCDQ
VDTLELSGGARINRIFHERFPFELVKMEFDEKDLRREISYAIKNIHGVRT
GLFTPDLAFEAIVKKQVVKLKEPCLKCVDLVIQELINTVRQCTSKLSSYP
RLREETERIVTTYIREREGRTKDQILLLIDIEQSYINTNHEDFIGFANAQ
QRSTQLNKKRAIPMQVIRRGWLTINNISLMKGGSKEYWFVLTAESLSWYK
DEEEKEKKYMLPLDNLKIRDVEKGFMSNKHVFAIFNTEQRNVYKDLRQIE
LACDSQEDVDSWKASFLRAGVYPEKDQAENEDGAQENTFSMPQLERQVET
IRNLVDSYVAIINKSIRDLMPKTIMHLMINNTKAFIHHELLAYLYSSADQ
SSLMEESADQAQRRDDMLRMYMALKEALNIIGDISTSTVSTPVPPPVDDT
WLQSASSHSPTPQRRPVSSIHPPGRPPAVRGPTPGPPLIPVPVGAAASFS
APPIPSRPGPQSVFANSDLFPAPPQIPSRPVRIPPGIPPGVPSRRPPAAP
SRPTIIRPAEPSLLD
Human Dynamin II Protein sequence - var3
(public gi: 1363934)

(SEQ ID NO: 97)

MGNRGMEELIPLVNKLQDAFSSIGQSCHLDLPQIAVVGGQSAGKSSVLEN
FVGRDELPRGSGIVTRRPLILQLIFSKTEHAEFLMCKSKKFTDFDEVRQE
IEAETDRVTGTNKCISPVPINLRVYSPHVLNLTLIDLPGITKVPVGDQPP
DIEYQIKDMILQFISRESSLILAVTPANMDLANSDALKLAKEVDPQGLRT
IGVITKLDLMDEGTDARDVLENKLLPLRRGYIGVVNRSQKDIEGKKDIRA
ALAAERKFFLSHPAYRHMADRMGTPHLQKTLNQQLTNHIRESLPALRSKL
QSQLLSLEKEVEEYKIFRPDDPTPKTKALLQMVQQFGVDPEKRIEGSGDQ
VDTLELSGGARINRIFHERFPFELVKMEFDEKDLRREISYAIKNIHGVRT
GLFTPDLAFEAIVKKQVVKLKEPCLKCVDLVIQELINTVRQCTSKLSSYP
RLREETERIVTTYIREREGRTKDQILLLIDIEQSYINTNHEDFIGFANAQ
QRSTQLNKKRAIPNQVIRRGWLTINNISLMKGGSKEYWFVLTAESLSWYK
DEEEKEKKYMLPLDNLKIRDVEKGFMSNKHVFAIFNTEQRNVYKDLRQIE
LACDSQEDVDSWKASFLRAGVYPEKDQAENEDGAQENTFSMDPQLERQVE
TIRNLVDSYVAIINKSIRDLMPKTIMHLMINNTKAFIHHELLAYLYSSAD
QSSLMEESADQAQRRDDMLRMYHALKEALNIIGDISTSTVSTPVPPPVDD
TWLQSASSMSPTPQRRPVSSIHPPGRPPAVRGPTPGPPLIPVPVGAAASF
SAPPIPSRPCPQSVFANSDLFPAPPQIPSRPVRIPPGIPPGVPSRRPPAA
PSRPTIIRPAEPSLLD
Rat Dynamin II mRNA sequence
(public gi: 6978770)

(SEQ ID NO: 155)

CGGTGAGGGGAGCCGGGCGGGTGGCAGAGCAGAGCAGGAGGAAGCAACGG
CCACAGACGCATCGGGACCGCGAGTCGTAGTCGGGGGATCCGCGGGGCGG
AGGCGGGCACCATGCGCAACCGCCGGATGGAAGAGCTCATCCCGCTGGTT
AACAAGCTGCAGGACGCCTTCAGCTCCATCGGTCAGAGCTGCCACCTGGA
CCTGCCGCAGATCGCCGTGGTGGGCGGCCAGAGCGCCGGCAAGAGTTCGG
TGCTCGAGAACTTCGTGGGCCGGGACTTCCTTCCACGAGGATCAGGAATT
GTCACCCGGAGGCCTCTCATTCTGCAGCTCATCTTTTCCAAAACAGAATA
TGCGGAGTTTTTGCACTGCAAGTCCAAAAAGTTTACAGACTTTGATGAAG
TCCGGCAGGAGATCGAAGCAGAGACTGACCGGGTCACAGGCACCAACAAA
GGCATCTCCCCGGTGCCCATCAACCTTCGGGTCTATTCACCACACGTGTT
GAACTTGACCCTCATCGACCTCCCAGGCATCACTAAGGTGCCAGTTGGGG
ACCAGCCACCAGACATCGAGTACCAGATCAAGGACATGATCCTGCAGTTT
ATCAGCCGGGAGAGCAGCCTCATTCTTGCCGTCACACCTGCCAACATGGA
CTTGGCCAACTCGGATGCCCTCAAGCTGGCCAAGGAGGTGGACCCCCAAG
GCTTGCGGACCATTGGTGTCATCACCAAGCTAGACCTGATGGATGAAGGC
ACAGATGCCAGGGATGTCCTGGAGAACAAGCTCCTGCCCTTGAGAAGAGG
CTACATCGGCGTGGTTAACCGCAGCCAGAAAGACATCGAGGGCAGAAAGG
ACATCCGGGCAGCTCTGGCAGCTGAGAGGAAATTCTTCCTCTCCCACCCA
GCCTACCGGCACATGGCTGACCGCATGGGTACCCCACACTTGCAGAAAAC
ACTGAACCAGCAACTGACCAACCACATCCGGGAGTCACTGCCAACCCTGC
GCAGCAAGCTGCAGAGCCAACTGCTGTCCCTGGAGAAGGAAGTGGAAGAG
TACAAGAATTTCCGGCCTGACGACCCCACACGCAAGACCAAAGCCCTGCT
GCAGATGGTCCAGCAGTTTGGAGTGGACTTTGAGAAGCGAATCGAAGGCT
CTGGAGATCAAGTCGACACCCTGGAGCTGTCTGGGGGCCCCCGCATCAAT
CGCATCTTCCACGAGCGCTTTCCCTTTGAGCTGGTGAAGATGGAGTTTGA
CGAGAAAGACCTCCGAAGAGAGATCAGCTACGCCATTAAGAACATCCACG
GAGTCAGAACTGGGCTCTTCACGCCGGATTTGGCGTTCGAGGCCATTGTG
AAAAAGCAGGTGGTCAAGCTGAAAGAGCCCTGTCTGAAATGCGTGGACCT
GGTTATCCAGGAGCTAATCAGTACAGTTAGGCAATGCACCAGCAAGCTCA
GTTCCTACCCCCGCCTTCGAGAGGAGACCGAACGCATTGTCACCACCTAC
ATCAGGGAGCGAGAAGGGAGAACCAAGGACCAGATCCTTCTTCTGATTGA
CATTGACCAGTCCTACATCAACACAAACCATGAAGACTTCATTGGATTTG
CCAATGCCCAGCAGAGGAGCACGCAGCTGAACAAGAAGAGGGCCATACCT
AACCAGGGGGAGATCTTGGTGATCCGCAGGGGCTGGTTGACCATCAACAA
CATCAGCTTGATGAAAGGCGGTTCCAAGGAGTACTGGTTCGTGCTGACTG
CTGAGTCGTTGTCTTCGTACAAGGATGAAGACGAAAAAGAAAAGAAGTAC
ATGCTGCCACTAGACAACCTCAAAATACGCGATGTGGAGAAGGGCTTCAT
GTCCAACAAGCATGTGTTTGCCATCTTCAACACAGAGCAGAGGAACGTCT
ACAAGGACCTGCGACAGATCGAACTGGCTTGTGACTCCCAGGAAGATGTG
GACAGCTGGAAGGCTTCGTTCCTGCGAGCTGGGGTCTACCCAGAGAAGGA
CCAGGCAGAGAACGAGGATGGAGCACAAGAGAACACCTTCTCCATGGACC
CGCAGCTGGAGCGGCAGGTGGAGACCATCCGCAACTTGGTGGACTCCTAT
GTGGCCATCATCAACAAGTCCATCCGGGACCTCATGCCAAAGACCATCAT
CCACCTCATGATCAACAACACAAAGGCCTTCATCCACCATGAGCTGCTAG
CCTACCTGTACTCATCAGCAGACCAGAGCAGCCTCATGGAGGAGTCAGCT
GAGCACGCTCAGCGGCGGGACGATATGCTGCGCATGTACCATGCACTCAA
GGAAGCGCTCAACATCATTGGGGACATCAGCACCAGCACCGTGTCCACGC
CTGTGCCCCCACCTGTCGATGATACTTGGCTCCAGAACACCAGCAGCCAC
AGCCCCACTCCACAGCGCAGACCTGTGTCCAGTGTGCACCCGCCGGGCCG
CCCACCCGCAGTGCGGGGCCCCACACCGGGGCCTCCCCTCATTCCTATGC
CTGTGGGGGCCACATCCTCCTTCTCGGCACCCCCCATCCCATCCCGGCCT
GGACCACAGAATGTGTTTGCCAACAATGACCCCTTCTCTGCGCCACCTCA
GATACCGTCTCGGCCAGCACGGATTCCCCCTGGGATCCCTCCAGGAGTGC
CCAGCAGAAGAGCGCCCGCTGCGCCCAGCCGGCCCACCATTATCCGCCCA
GCCGAGCCGTCCCTGCTCGACTAGGCTGCAGGGGACAGTGTTCTGGGCGG
GTCCTCGTGTACCTACAGTGGTCTGGGCCCTGCTACTGCCCCCCATGCTG
GGACCAGGCTCCCCGTGGGCAAGCCTGCCCTCTCCTCCCTAGCCCTAGTG
CACATGATAAGGAGGGAGAGGGTGGGAAGGAGGCCCAAACAAACAGAACT
TGGGGGCTTTGCACTTTGGGGCGAGGGCGAGCTCTGGTTAGCAGAACAGA
AACTGCACCCTTGGGACCATCTGGAAAGAAAGGTCTAGGCTAGTGGTGGG
GAACTCTACATGGGCTCCTCCATACTGTAAGCCTTTGCTCTTTCTGAGGT
ACAGAACTGTCCACCTTTCCCTAAGGACTCCTCAGGAGTTAGCCAGGCCC
AGGAGGTCCAGTGCTGGCCACCCACCTGGCTCAAGTTGTATATATATATA
TAGCTTCTTTGGCCACATTAACCACACAGCCTGAGCCCAGCCCAGTCTCC
ACACCCAGAGGTGCCTTTGTGCCTTCCAGGCCTGGAGTGCCTCAGCTCTG
GCCTGCCTCTCTCCTCAGCCCCTGTTGTGGTTTGGGCTGTAAGAACTCTC
GAGACATCAGGACCTCTTGGCTTCCAACCAGATGTCTCCTACAGGGTGGG
CCCTGGTGCCTCTGTACACATTCTGCTCCCAACGCTTGGATTGACCACTG
TTAAGTGCCTGCCTCTGTATATCCTATTAATAAACTAAAATAAAGGGAAG
CCCTCTCGTCGCT
Rat Dynamin II Protein sequence
(public gi: 6978771)

(SEQ ID NO: 156)

MGNRGMEELIPLVNKLQDAFSSIGQSCHLDLPQIAVVGGQSAGKSSVLEN
FVGRDPLPRGSGIVTRRPLILQLIFSKTEYAEFLHCKSKKFTDFDEVRQE
IEAETDRVTGTNKGISPVPINLRVYSPHVLNLTLIDLPGITKVPVGDQPP
DIEYQIKDMILQFISRESSLILAVTPANMDLANSDALKLAKEVDPQGLRT
IGVITKLDLMDEGTDARDVLENKLLPLRRGYIGVVNRSQKDIEGRKDIRA
ALAAERKFFLSHPAYRHMADRNGTPMLQKTLNQQLTNHIRESLPTLRSKL
QSQLLSLEKEVEEYKNFRPDDPTRKTKALLQMVQQFGVDFEKRIEGSGDQ
VDTLELSGGARINRIFIERFPFELVKMEFDEKDLRREISYAIKNIHGVRT
GLFTPDLAFEAIVKKQVVKLKEPCLKCVDLVIQELISTVRQCTSKLSSYP
RILREETERIVTTYIREREGRTKDQILLLIDIEQSINTNHEDFIGFANAQ
ORSTQLNKKRAIPNQGEILVIRRGWLTINNISLMKGGSKEYWFVLTAESL
SWYKDEEEKEKKYMLPLDNLKIRDVEKGFMSNKHVFAIFNTEQRNVYKDL
RQIELACDSQEDVDSWKASFLRAGVYPEKDQAENEDGAQENTFSMDPQLE
RQVETIRNLVDSYVAIINKSIRDLMPKTIMHLMINNTKAFIHMELLAYLY
SSADQSSLMEESAEQAQRRDDMLRNYHALKEALNIIGDISTSTVSTPVPP
PVDDTWLQNTSSHSPTPQRRPVSSVHPPGRPPAVRGPTPGPPLIPMPVGA
TSSFSAPPIPSRPGPONVFANNDPFSAPPQIPSRPARIPPGIPPGVPSRR
APAAPSRPTIIRPAEPSLLD
Mouse Dynamin II mRNA sequence
(public gi: 6681206)

(SEQ ID NO: 157)

GAATTCAAGCGCCTGCCGCGACAGGGGAGACCGGGCGGGCCGCAGAGCAG
AGCAGGAGGAAGCAGCGGCCACAGACACATCGGAACCGAGAGTCTTAGTC
GGGGGATCCGCGCGGCGAGGCGGACACCATCGGCAACCGCGGGATGGAAG
AGCTCATCCCGCTGGTTAACAAGCTGCAGGACGCCTTCAGCTCCATCGGT
CAGAGCTGCCACCTGGACCTGCCGCAGATCGCCGTGGTGGGCGGCCAGAG
CGCCGGCAAAAGTTCGGTGCTCGAGAACTTCGTCCGCCGGGACTTCCTTC
CACGAGGATCAGGAATTGTCACCCGGAGGCCTCTCATTCTGCAGCTCATC
TTTTCCAAAACAGAATATGCGGAATTTTTGCACTGCAAGTCCAAAAAATT
TACAGACTTTGATGAAGTCCGCCAGGAGATCGAAGCAGAGACTGACCGGG
TCACAGGCACCAACAAAGGCATCTCCCCCGTGCCCATCAACCTTCGGGTC
TACTCACCACACGTGTTGAACTTGACCCTCATCGACCTCCCACGCATCAC
TAAGGTGCCGGTGGGGGACCAGCCGCCAGACATCGAGTACCAGATCAAGG
ACATGATCCTGCAGTTCATCAGCCGCGAGAGCAGCCTCATTCTTGCCGTC
ACACCTGCCAACATGGACTTGGCCAACTCAGACGCCCTCAAGCTCGCCAA
GGAGGTGGACCCCCAAGGCCTACCGACCATCGGTGTCATCACCAAGCTAG
ACTTGATGGATGAAGGCACAGACGCCAGGGATGTCCTGGAAAACAAGCTA
CTGCCCTTGAGAAGAGGCTATATCGGCGTGGTTAACCGAAGCCAGAAAGA
CATCGAGGGCAAAAAGGACATCCGGGCTGCTCTGGCAGCCGAGAGGAAAT
TCTTCCTCTCCCACCCAGCCTACCGGCACATGGCTGACCGCATGGGCACC
CCACACTTGCAGAAAACCCTGAACCAGCAACTGACCAACCACATCCGAGA
GTCACTGCCGACCCTTCACGGCAAGCTGCAGAGCCAACTGCTGTCCCTGG
AGAAGGAAGTGCAAGAGTACAAGAATTTCCGGCCTGATGACCCCACGCGC
AAGACCAAAGCCCTGCTGCAGATGGTTCAGCAGTTTGGAGTGGACTTTGA
GAAGCGAATTGAAGGCTCGGGAGATCAAGTAGACACACTAGAGTTGTCTG
GTGGAGCCCGCATCAATCGTATCTTTCATGAGCGCTTTCCCTTTGAACTG
GTAAAGATGGAGTTTGATGAGAAAGATCTACGAAGAGAGATCAGCTATGC
TATTAAGAACATCCACGGAGTCAGAACTGGGCTCTTCACGCCGGATTTGG
CGTTCGAGGCCATTGTGAAAAAGCAGGTGGTCAAGCTGAAAGAGCCCTGT
CTGAAATGCGTGGACCTGGTTATCCAGGAGCTAATCAGTACAGTTACGCA
GTGCACCAGCAAGCTGAGTTCCTACCCCCGGCTGCGAGAGGAGACCGAGC
GAATTGTCACCACCTACATCAGGGAGCGAGAAGGGAGAACCAAGGACCAG
ATTCTTCTTCTGATTGACATTGAGCAGTCGTACATCAACACAAACCATGA
AGACTTCATTGGATTTGCCAATGCCCAGCAGAGGAGCACGCAGCTGAACA
AGAAGAGGGCCATACCCAATCAGGTGATCCGCAGGGGCTGGTTGACCATC
AACAATATCAGCTTGATGAAGGGTGGCTCCAAGGAGTACTGGTTCGTGCT
GACAGCTGAGTCATTGTCTTGGTACAAGGATGAGGAGGAGAAAGAAAAGA
AGTACATGCTGCCACTGGACAACCTCAAGATCCGGGACGTGGAAAAGGGC
TTCATGTCCAACAAGCACGTGTTTGCCATCTTCAACACAGAGCAGAGGAA
TGTCTACAAGGACCTTCGACAGATTGAACTGGCTTGTGACTCCCACGAAG
ATGTGGACAGCTGGAAGGCTTCATTCCTGCGCGCTGGGGTCTACCCCGAG
AAGGACCAGGCAGAGAATGAGGATGGAGCACAAGAGAACACCTTCTCCAT
GGACCCGCAGCTGGAGCGACAGGTGGAGACCATCCGTAACCTGGTGGACT
CCTACGTGGCCATCATCAACAAGTCCATCCGTGACCTTATGCCAAAGACC
ATCATGCACCTCATGATCAACAACACAAAGGCCTTCATCCACCATGAGCT
GCTGGCTTACCTGTATTCATCAGCAGACCAGAGCAGCCTCATGGAAGAGT
CAGCCGAGCAGGCTCAGCGGCGGGACGACATGCTGCGCATGTACCACGCA
CTGAAGGAGGCCCTCAACATTATTGGGGACATCAGCACCAGCACCGTGTC
CACACCCGTGCCCCCACCGGTCGATGACACGTGGCTCCAAAACACCAGCG
GCCACAGCCCCACTCCACAGCGCCGACCTGTGTCCAGTGTGCACCCACCA
GGCCGGCCACCTGCAGTGCGGGGTCCTACACCGGOGCCTCCCCTGATTCC
TATGCCTGTGGGGGCCACATCCTCCTTCTCAGCACCTCCCATCCCATCCC
GGCCTGGACCACAGAGTGTGTTTGCCAACAACGACCCCTTCTCTGCGCCA
CCTCAGATACCATCTCGGCCAGCACGGATTCCCCCTGGTATCCCTCCAGG
AGTGCCCAGCAGAAGAGCGCCCGCTGCGCCCAGCCGGCCCACCATTATCC
GCCCAGCCGAGCCGTCCCTGCTCGACTAGGCTGCAGGGGGCAATGTTCTG
GGGGGGTCCTCGTGCACCCACAGTGTAGGACAGCTTTGGTGGTCTGGGCC
CTGCTACTTGCCCTCCATGCTGGGACCAGGCTCCCCGTGCAAGCCCTGCC
CTTTCCCTCCTCGCCCTAGTGGACATGACAATGAAGGGTGAGGAGGCCCA
CAGCACAGCACATGGGGGCTTTGCACTTTGGGATGAGGAGGAGCTCTGGC
TAGCAGAACAGGAACTGCATCCTTGGGACCATCAGGAAAAAAAGGTCCAG
GCTAGTGTGGGGGGACTCTTCTGGAGCTCCTCCAGACCCTTTGTTCTTTC
TGGGGTCCAGGAACTGCCCACCTTTCCTAAGGACTCCTCAGGAGTGAGCC
ACGCCCAGCAGGTCCAGTGCTGACCCACCTGGCTCAAGTTGTATATATAG
AGCTCCTTTGGCCATATTAACCACACAAGCTGAGCCCAGCCCAGGCTCCG
ACCCAGACGTGCCTTTGTGCCTTCCTCAGCCTGGAGGGCCTCAGCTCTGG
CCACCCTCACTCTCCTCAGCCCCGGTTGTGGTTTGGGCTGTATGAACTGG
GGAGACATCAGAGGCCTCTTGGCTCCCAACCAGATGTCCCACAGGGTGGG
CCCTGGGTGCCTCTGCAGCCTCGGCTCCCACCGCCTGGATTGACCACTGT
TAAGTGCCTGCCTCTGTATATCCTATTAATAAACTAAAATAAAGGGAAAA
AAAAGCTT
Mouse Dynamin II Protein sequence
(public gi: 6681207)

(SEQ ID NO: 158)

MGNRGMEELIPLVNKLQDAFSSIGQSCHLDLPQIAVVCGQSAGKSSVLEN
FVGRDFLPRGSGIVTRRPLILQLIFSKTEYAEFLMCKSKKFTDFDEVRQE
IEAETDRVTGTNKGISPVPINLRVYSPHVLNLTLIDLPGITKVPVGDQPP
DIEYQIKDMILQFISRESSLILAVTPANNDLANSDALKLAKEVDPQGLRT
IGVITKLDLMDEGTDARDVLENKLLPLRRGYIGVVNRSQKDIEGKKDIRA
ALAAERKPFLSHPAYRHMADRNGTPHLQKTLNQQLTNHIRESLPTLHGKL
QSQLLSLEKEVEEYKNFRPDDPTRKTKALLQMVQQFGVDFEKRIEGSGDQ
VDTLELSGGARINRIFHERFPFELVKMEFDEKDLRREISYAIKNIHGVRT
GLFTPDLAFEAIVKKQVVKLKEPCLKCVDLVIQELISTVRQCTSKLSSYP
RLREETERIVTTYIREREGRTKDQILLLIDIEQSYINTNHEDFIGFANAQ
QRSTQLNKKRAIPNQVIRRGWLTINNISLMKGGSKEYWFVLTAESLSWYK
DEEEKEKKYMLPLDNLKIRDVEKGFMSNKHVFAIFNTEQRNVYKDLRQIE
LACDSQEDVDSWKASFLRAGVYPEKDQAENEDGAQENTFSMDPQLERQVE
TIRNLVDSYVAIINKSIRDLMPKTIMHLMINNTKAFIHHELLAYLYSSAD
QSSLMEESAEQAQRRDDMLRNYHALKEALNIIGDISTSTVSTPVPPPVDD
TWLQNTSGHSPTPQRRPVSSVHPPGRPPAVRGPTPGPPLIPMPVGATSSF
SAPPIPSRPGPQSVFANNDPFSAPPQIPSRPARIPPGIPPGVPSRRAPAA
PSRPTIIRPAEPSLLD
Drosophila Dynamin II mRNA sequence
(public gi: 24642335)

(SEQ ID NO: 159)

GATCTGATATCTAATCTAATCTAATTGTGAGGGAAATCGGACCTCGCCGC
AATGGATAGTTTAATTACAATTGTTAACAAGCTGCAAGATGCGTTCACAT
CGCTCGGCGTCCACATGCAACTGGATCTGCCGCAGATCGCGCTGGTCGGT
GGCCAGTCAGCTGGCAAGAGTTCCGTTTTGGAGAACTTTGTGGGCAAAGA
TTTCTTGCCGCGCGGCTCTGGTATCGTAACTCGGAGACCACTAATCTTGC
AACTGATCAATGGAGTTACCGAATATGGCGAGTTCCTTCACATTAAAGCC
AAGAAGTTTTCCAGCTTCGATGAGATCAGAAAAGAAATCGAAGATGAAAC
GGATCGTGTGACCCGTAGTAACAAAGGCATTTCAAACATTCCAATTAATT
TGCGCGTCTACTCGCCGCACGTACTCAATTTAACCCTCATCGATTTGCCA
GGCCTGACAAAGGTGGCCATTGGCGATCAACCGGTGGATATTGAGCAGCA
AATTAAGCAGATGATATTCCAATTTATTCGCAAAGAGACTTGTTTAATTT
TGGCTGTGACCCCGGCCAATACGGATCTGGCCAATTCGGATGCCCTCAAG
CTGGCCAAGGAGGTGGATCCACACGGTGTGCGAACCATTGGCGTTATAAC
CAAGCTGGATCTGATGGACGAGGGCACCGATGCACGCGACATTCTGGAGA
ATAAGCTGCTTCCGCTGCGACGTGGCTATATTGGCGTGGTGAATAGATCC
CAAAAGGATATCGAGGGGCGCAAGGATATCCACCAGGCATTGGCCGCCGA
ACGCAAGTTCTTCTTGAGCCATCCCTCCTATCGGCACATGGCCGACCGTC
TCGGCACCCCCTACTTGCAGCGCGTGCTCAACCAACAACTAACCAATCAC
ATCAGGGACACGTTACCTGGCCTGCGGGATAAGTTGCAGAAGCAGATGCT
CACTTTGGAGAAGGAGGTGGAGGAGTTTAAGCACTTTCAGCCAGGCGATG
CTAGCATCAAAACAAAAGCTATGCTCCAAATGATTCAGCAGCTGCAATCA
GACTTTGAGCGAACTATCGAGGGCAGCGGCTCAGCTTTGGTGAACACAAA
CGAGCTCTCAGGTGGCGCCAAGATCAATCGCATTTTCCATGAACGTCTGC
GCTTTGAGATCGTAAAGATGGCTTGCGATGAAAAGGAACTGCGACGAGAG
ATCTCATTTGCAATTCGAAATATCCATGGTATTCGCGTTGGCCTCTTCAC
ACCCGACATGGCCTTCGAGGCCATTGTCAAGCGGCAGATACCGCTGCTCA
AGGAGCCGGTTATCAAGTGTGTCGATCTAGTCGTACAGGAACTTTCTGTG
GTCGTGCGCATGTGCACAGCTAAAATGAGTCGATACCCACGTCTGCGTGA
GGAGACCGAACGTATTATCACAACACATGTCCGCCAACGCGAGCATAGCT
GCAAGGAACAGATCCTCCTGCTTATTGACTTTGAGCTGGCCTATATGAAC
ACCAATCATGAGGACTTCATTGGCTTCGCCAACGCTCAAAACAAATCAGA
GAATGCCAACAAAACTGGCACTCGTCAACTGGGCAACCAAGTCATTCGCA
AGGGTCACATGGTCATCCAGAACCTTCGAATCATGAAAGGTGGATCGCGT
CCGTATTGGTTTGTGCTGACATCCGAGAGTATCTCCTGGTATAAGGACGA
GGATGAGAACGAGAAGAAATTTATGCTGCCGTTGGATGGTTTAAAATTGC
GTGATATTGAACAGGGATTTATGTCAATGTCTAGACGTGTTACATTTGCT
TTATTTAGTCCCGATGGACGTAATGTTTATAAGGACTACAAACAACTGGA
GCTCTCGTGCGAGACTGTGGAGGATGTGGAGTCCTGGAAAGCATCCTTCC
TGCGAGCTGGTGTCTATCCCGAAAAGCAGGAGACTCAGGAAAACGGCGAT
GAGTCGGCCAGCGAGGAGAGTTCCAGCGATCCACAGCTGGAGCGGCAGGT
CGAGACCATTCGTAATCTGGTTGATTCCTACATGAAGATCGTTACGAAGA
CCACCCGTGACATGGTGCCCAAAGCAATTATGATGCTGATCATCAACAAT
GCCAAGGACTTTATCAATGGCGAGCTGCTGGCACATCTTTACGCGTCCGG
CGATCAGGCCCAGATGATGGAGGAGTCGGCTGAATCGGCAACACGGCGAG
AGGAAATGCTGCGCATGTACCGTGCTTGCAAGGATGCATTACAGATAATA
GGTGACGTATCAATGGCCACCGTGTCGTCGCCATTGCCACCGCCGGTTAA
GAACGATTGGCTGCCCAGCGGCTTGGACAACCCACGTCTTTCTCCTCCAA
GTCCGGGTGGCGTTCGAGGAAAGCCAGGACCTCCCGCACAGAGCTCCTTG
GGTGGACGGAACCCACCATTGCCACCATCTACAGGTCGCCCAGCGCCAGC
GATTCCGAATCGCCCAGGAGGTGGGGCACCACCACTACCCGGCGGTCGTC
CCGGAGGATCTCTTCCCCCACCAATGCTACCATCCCGTGTGTCCGGAGCT
GTCGGCGGCGCCATTGTCCAGCAATCGGGAGCCAATCGCTATGTGCCGGA
GACTATGCGTGGACAGGTGAACCAGGCGGTGGGCCAGGCAGCCATCAATG
AGTTGTCCAATGCCTTCAGTTCGCGATTCAAGTAATGGCCGCGTAATTGA
GTGCACAGCATGCCAGAGTATGCTTAATTATTAACAGAAACAGAAACTTA
GAGAGCCAGCACGCGAAACCCCTCCCCCCACCAACCATATCACATCATTT
TTTTTTTTATCAAAGATGTAAATCCCAAAAAGATGGCAATTCCCATCCAG
TTGTTTCGAAAATTCGATGGAAAAATTTAAAAAGTGTTTTACACATGATC
TTTTCGACAATTCAACATTCAAATTAGTGCTTTCGAATTAAGTTTATCGG
GGATACCATTTACTTTTTAAAGTATATTTGACGTGAATAAAAAAAAAAAA
AAATTTATTTTCC
Drosophila Dynamin II Protein sequence
(public gi: 24642336)

(SEQ ID NO: 160)

MDSLITIVNKLQDAFTSLGVHMQLDLPQIAVVGGQSAGKSSVLENFVGKD
FLPRGSGIVTRRPLILQLINGVTEYGEFLHIKGKKFSSFDEIRKEIEDET
DRVTGSNKCISNIPINLRVYSPHVLNLTLIDLPGLTKVAIGDQPVDIEQQ
IKQMIFQFIRKETCLILAVTPANTDLANSDALKLAKEVDPQGVRTIGVIT
KLDLMDEGTDARDILENKLLPLRRGYIGVVNRSQKDIEGRKDIHQALAAE
RKFFLSHPSYRHMADRLGTPYLQRVLNQQLTNMIRDTLPGLRDKLQKOML
TLEKEVEEFKHFQPGDASIKTKANLOMIOQLQSDFERTIEGSGSALVNTN
ELSGGAKINRIFMERLRFEIVKMACDEKELRREISFAIRNIHGIRVGLFT
PDMAFEAIVKRQIALLKEPVIKCVDLVVQELSVVVRMCTAKMSRYPRLRE
ETERIITTHVRQREMSCKEQILLLIDFELAYMNTNHEDFIGFANAQNKSE
NANKTGTRQLGNQVIRKGHMVIQNLGIMKGGSRPYWFVLTSESISWYKDE
DEKEKKFMLPLDGLKLRDIEQGFMSMSRRVTFALFSPDGRNVYKDYKQLE
LSCETVEDVESWKASFLRAGVYPEKQETQENGDESASEESSSDPQLERQV
ETIRNLVDSYMKIVTKTTRDMVPKAIMNLIINNAKDFINGELLAHLYASG
DQAQMMEESAESATRREEMLRNYRACKDALQIIGDVSMATVSSPLPPPVK
NDWLPSGLDNPRLSPPSPCGVRGKPGPPAQSSLGGRNPPLPPSTGRPAPA
IPNRPGGGAPPLPGGRPGGSLPPPMLPSRVSGAVGGAIVQQSGANRYVPE
SMRGQVNQAVGQAAINELSNAFSSRFK
C. elegans Dynamin II mRNA sequence
(public gi: 25146622)

(SEQ ID NO: 161)

CAGCCATGTCGTGGCAAAACCAGGGAATGCAGGCGTTGATCCCTGTGATC
AATCGTGTCCAGGACGCCTTCTCCCAGCTGGGCACATCTGTCAGCTTCGA
ACTTCCACAGATCGCCGTCGTCGGAGGACAGTCCGCTGGAAAGTCGTCGG
TGCTGGAGAATTTTGTCGGAAAAGACTTCTTGCCACGTGGATCAGGAATC
GTAACACGTCGTCCACTTATTTTGCAGCTTATTCAAGATCGCAATGAGTA
CGCCGAGTTCCTACACAAGAAGGGTCATCGCTTTGTCGATTTTGATGCAG
TTCGGAAAGAGATTGAGGATGAGACTGATCGTGTCACTGGACAGAATAAG
GGAATCAGTCCACATCCAATCAACTTGCGTGTCTTTTCTCCAAATGTTCT
AAATCTGACACTCATCGATTTGCCCGGTCTCACAAAAGTGCCCGTCGGAG
ATCAACCAGCAGATATTGAGCAACAGATCCGTGACATGATTCTCACATTC
ATCAACCGTGAGACTTGCCTCATTCTTGCCGTCACTCCGGCCAACAGCGA
TCTCGCCACTTCGGATGCGTTGAAACTTGCGAAGGAAGTCGATCCACAGG
GTCTTCGCACGATTGGAGTCCTCACCAAACTTGACTTGATGGACGAGGGA
ACCGATGCTCGCGAGATCCTCGAGAACAAGCTGTTCACACTTCGTCGTGG
CTACGTCGGAGTTGTCAATCGTGGGCAGAAGGATATTGTCGGTCGCAAGG
ATATTAGAGCTGCTTTGGACGCCGAGAGAAAGTTCTTCATCTCACACCCA
TCCTACCGACATATGGCTGATCGGTTGGGAACAAGCTACCTTCAACACAC
TCTTAATCAACAGCTCACCAATCATATCCGTGATACACTGCCAACACTTC
GTGATAGTCTTCAAAAGAAGATGTTTGCTATGGAAAAGGATGTGGCCGAG
TACAACAACTACCAGCCAAATGATCCAGGCCGCAAGACCAAGGCTCTTTT
GCAAATGGTTACCCAGTTCAATGCTGACATTGAGCGCTCCATTGAAGGTT
CCTCTGCAAAGCTGGTTTCAACCAATGAGCTCAGTGGAGGAGCCCGTATC
AATCGGCTTTTCCATGAGCGTTTCCCATTTGAGATTGTTAAAATGGAAAT
TGACGAGAAAGAAATGCGCAAAGAAATCCAGTATGCCATCAGAAACATTC
ACGGTATCCGCGTCGGTCTCTTCACTCCGGATATGGCGTTTGAGGCAATT
GCCAAAAAGCAAATCACCCGTCTGAAGGAGCCATCGTTGAAATGCGTTGA
TCTGGTGGTCAACGAGTTGGCTAATGTGATCAGACAGTGCGCTGACACTA
TGGCTAGATATCCACGTCTTCGTGACGAGCTGGAAAGAATCGTCGTCTCG
CATATGCGTGAACGTGAGCAAATTGCCAAGCAGCAAATTGGGCTCATTGT
TGACTACGAACTCGCTTATATGAACACAAACCATGAGGATTTCATTGGAT
TCAGCAATGCTGAAGCAAAAGCCTCCCAAGGACAATCAGCGAAGAAGAAT
CTTGGAAACCAGGTGATCAGAAAGGGCTGGCTCTCACTGAGCAACGTATC
GTTTGTGCGTGGCTCCAAGGACAATTGGTTTGTGCTCATGTCGGACAGTT
TGAGTTGGTACAAAGATGATGAGGAGAAGGAGAAGAAGTACATGCTCCCA
TTGGATGGTGTCAAGCTGAAGGATATTGAGGGTGGATTTATGTCTCGTAA
CCACAAGTTTGCTCTGTTCTACCCCGACGGAAAGAACATCTACAAGGATT
ACAAGCAGCTTGAGTTGGGATGCACCAATTTGGACGAAATTGATGCGTGG
AAGGCTTCATTCTTGCGTGCTGGTGTCTATCCAGAAAAGCAGAAGGCACA
GGAAGATGAGTCCCAACAAGAGATGGAGGATACCTCGATTGATCCACAAC
TTGAGAGACAGGTGGAGACAATCCGTAATTTGGTTGATTCCTACATGAGA
ATCATTACCAAGACAATTAAGGACCTGGTTCCAAAGGCGGTGATGCATTT
GATTGTTAACCAGACAGGTGAGTTCATGAAAGATGAACTTTTGGCCCATC
TCTACCAATGCGGCGACACTGATGCTCTCATGGAGGAATCTCAAATAGAA
GCCCAGAAGCGCGAGGAGATGCTCCGAATGTACCATGCTTGCAAGGAGGC
GCTCCCGATTATCTCTGAAGTCAACATGAGCACCCTTGGCGACCAGCCGC
CGCCATTGCCGATGTCTGACTACCGCCCACACCCATCTGGACCTTCACCG
GTGCCGCGTCCGGCTCCTGCTCCACCAGGCGGACGTCAGGCCCCAATGCC
ACCACGCGGAGGACCCGGTGCCCCACCACCACCAGGCATGAGACCACCAC
CAGGTGCGCCAGGAGGCGGCGGTGGCATGTACCCACCGTTGATTCCAACG
CGTCCGGGACCAGGCGGCCCACCACCCAACATGGCACCGCCTAGATAATA
ATGTTGTGAATAATTCCCCCACTGACCAAAGAAGAGTACCGACACCATCC
AACGGAGCACCAGAGATTCCAGCTCGCCCGCAAGTCCCCAAGAGACCTTT
CTAAATGCTTTTACATCTCCATCCTAGCTCACTTGTCATACTATTACCGT
TCCCATAGTAATTGTACAATGCCCCCGTTTTATCAAGCATTTCTTTTTTT
TTTTTGAGAATTTTTAAATTAATCAATATCCCGAGTTCTTCCACCTTTTG
AGCAAGTCTCGTTGAGGATTGCATTGGTCCCAGCGCTAACTAGGCGTCAA
TTCATCCACCTCTCCATTCTCTCTCCCGTTCCAACTGTGTTTTTCTTTGT
CAGCGGCGAATTTTCCACAACCAATCGAGTCCTATTTCTCTTTTTTTCCC
CCACCTCCCAATAATTTTATTTGCATGTTTTATAAACTCACTTTGTTCAA
TAATTTATTGATTTTATTTTTTCTATGTATTTGAAACTTTACATTTTTCC
TACTCTTCTATAGCAATACTTTGTCACTTACAATAACCCCCCCCCCCCCA
GTCTGCCTATATTTATTTAGATGTTCTTTATGGCCACCCAATGTATAAAC
TCTTTTAGTGCATTTGTAGGTTCTTTCTTCATCCCACCCAATTCCCCCTC
CACCACAAATCCCAGTTTTGATTATTTTAAGCCCATATTTATAAGAAGTA
AATATATTTATAGATTTTTCTTAGATTATAAGCTCTTCTAGGTTTTCCCT
CGTTGAGAAGAATGATCTGAAAAAGCCTAAATTATAGATTTTTGAAACGT
ATTGTGTAGAATTTGTATTTATTCGTGAAAGTGGAATAAAGTTAAAACGG
C. elegans Dynamin II Protein sequence
(public gi: 25146623)

(SEQ ID NO: 162)

MSWQNQGMQALIPVINRVQDAFSQLGTSVSPELPQIAVVGGQSAGKSSVL
ENFVGKDFLPRGSCIVTRRPLILQLIQDRNEYAEFLHKKGHRFVDFDAVR
KEIEDETDRVTGQNKGISPHPINLRVFSPNVLNLTLIDLPGLTKVPVGDQ
PADIEQQIRDMILTFINRETCLILAVTPANSDLATSDAIKLAKEVDPQGL
RTIGVLTKIDLMDEGTDAREILENKLFTLRRGYVGVNRGQKDIVGRKDIR
AALDAERICFFISHPSYRHMADRLGTSYLQHTLNQQLTNMIRDTLPTLRD
SLQKKMFANEKDVAEYKNYQPNDPGRKTKALLQMVTQFNADIERSIEGSS
AKLVSTNELSGGARINRLFHERFPFEIVKMEIDEKEMRKEIQYAIRNIHG
IRVGLFTPDMAFEAIAKKQITRLKEPSLKCVDLVVNELANVIRQCADTMA
RYPRLRDELERIVVSHMREREQIAKQQIGLIVDYELAYMNTNHEDFIGFS
NAEAKASQGQSAKKNLGNQVIRKGWLSLSNVSFVRGSKDNWFVLMSDSLS
WYKDDEEKEKKYMLPLDGVKLKDIEGGFMSRNHKFALFYPDGKNIYKDYK
QLELGCTNLDEIDAWKASFLRAGVYPEKQKAQEDESQQEMEDTSIDPQLE
RQVETIRNLVDSYMRIITKTIKDLVPKAVMHLIVNQTGEFMKDELLAHLY
QCGDTDAIMEESQIEAQKREEMLRMYHACKEALPIISEVNMSTLGDQPPP
LPMSDYRPHPSGPSPVPRPAPAPPGGRQAPMPPRGGPGAPPPPCMRPPPG
APGGGGGMYPPLIPTRPGPGGPPPNMAPPR
Below are sequences representative of the polypep-
tide. POSH:
Human POSH Coding Sequence

(SEQ ID NO: 1)

ATGGATGAATCAGCCTTCTTGGATCTTTTGGAGTGTCCGGTGTGTCTAGA
GCGCCTTGATGCTTCTGCGAAGGTCTTGCCTTGCCAGCATACGTTTTGCA
AGCGATGTTTGCTGGGGATCGTAGGTTCTCGAAATGAACTCAGATGTCCC
GAGTGCAGGACTCTTGTTGGCTCGGGTGTCGAGGAGCTTCCCAGTAACAT
CTTGCTGGTCAGACTTCTGGATGGCATCAAACAGAGGCCTTGGAAACCTG
GTCCTGGTGGGGGAAGTGGGACCAACTGCACAAATGCATTAAGGTCTCAG
AGCAGCACTGTGGCTAATTGTAGCTCAAAAGATCTGCAGAGCTCCCAGGG
CGGACAGCAGCCTCGGGTGCAATCCTGGAGCCCCCCAGTGAGGGGTATAC
CTCAGTTACCATGTGCCAAAGCGTTATACAACTATGAAGGAAAAGAGCCT
GGAGACCTTAAATTCAGCAAAGGCGACATCATCATTTTGCGAAGACAAGT
GGATGAAAATTGGTACCATGGGGAAGTCAATGGAATCCATGGCTTTTTCC
CCACCAACTTTGTGCAGATTATTAAACCGTTACCTCAGCCCCCACCTCAG
TGCAAAGCACTTTATGACTTTGAAGTGAAAGACAAGGAAGCAGACAAAGA
TTGCCTTCCATTTGCAAAGGATGATGTTCTGACTGTGATCCGAAGAGTGG
ATGAAAACTGGGCTGAAGGAATGCTGGCAGACAAAATAGGAATATTTCCA
ATTTCATATGTTGAGTTTAACTCGGCTCCTAAGCAGCTGATAGAATGGGA
TAAGCCTCCTGTGCCAGGAGTTGATGCTGGAGAATGTTCCTCGGCAGCAG
CCCAGAGCAGCACTGCCCCAAAGCACTCCGACACCAAGAAGAACACCAAA
AAGCGGCACTCCTTCACTTCCCTCACTATGGCCAACAAGTCCTCCCAGGC
ATCCCAGAACCGCCACTCCATGGAGATCAGCCCCCCTGTCCTCATCAGCT
CCAGCAACCCCACTGCTGCTGCACGGATCAGCGACCTGTCTGGGCTCTCC
TGCAGTGCCCCTTCTCAGGTTCATATAAGTACCACCGGGTTAATTGTGAC
CCCGCCCCCAAGCACCCCAGTGACAACTGGCCCCTCGTTTACTTTCCCAT
CAGATGTTCCCTACCAAGCTGCCCTTGGAACTTTGAATCCTCCTCTTCCA
CCACCCCCTCTCCTGGCTGCCACTGTCCTTGCCTCCACACCACCAGGCGC
CACCGCCGCCGCTGCTGCTGCTGGAATGGGACCGAGGCCCATGGCAGGAT
CCACTGACCAGATTGCACATTTACGGCCGCAGACTCGCCCCAGTGTGTAT
GTTGCTATATATCCATACACTCCTCGGAAAGAGGATGAACTAGAGCTGAG
AAAAGGGGAGATGTTTTTAGTGTTTGAGCGCTGCCAGGATGCCTGGTTCA
AAGGGACATCCATGCATACCAGCAAGATAGGGGTTTTCCCTGGCAATTAT
GTGGCACCAGTCACAAGGGCGGTGACAAATGCTTCCCAAGCTAAAGTCCC
TATGTCTACAGCTGGCCAGACAAGTCGGGGAGTGACCATGGTCAGTCCTT
CCACGGCAGGAGGGCCTGCCCAGAAGCTCCAGGGAAATGGCGTGGCTGCG
AGTCCCAGTGTTGTCCCCGCAGCTGTGGTATCAGCAGCTCACATCCAGAC
AAGTCCTCAGGCTAAGGTCTTGTTGCACATGACGGGGCAAATGACAGTCA
ACCAGGCCCGCAATCCTGTGACGACAGTTGCAGCGCACAACCAGGAACGC
CCCACGGCAGCAGTGACACCCATCCAGGTACAGAATGCCGCCGGCCTCAG
CCCTGCATCTGTGGGCCTGTCCCATCACTCGCTGGCCTCCCCACAACCTG
CGCCTCTGATGCCAGGCTCAGCCACGCACACTGCTGCCATCAGTATCAGT
CGAGCCAGTGCCCCTCTGGCCTGTGCAGCAGCTGCTCCACTGACTTCCCC
AAGCATCACCAGTGCTTCTCTGGAGGCTGAGCCCAGTGGCCGGATAGTGA
CCGTTCTCCCTCGACTCCCCACATCTCCTGACAGTGCTTCATCAGCTTGT
GGGAACAGTTCAGCAACCAAACCAGACAAGGATAGCAAAAAAGAAAAAAA
GGGTTTGTTGAAGTTGCTTTCTGGCGCCTCCACTAAACGGAAGCCCCGCG
TGTCTCCTCCACCATCGCCCACCCTAGAAGTGGAGCTGGGCAGTGCAGAG
CTTCCTCTCCAGGGAGCGGTGGGGCCCGAACTGCCACCAGGAGGTGGCCA
TGGCAGGGCAGGCTCCTGCCCTGTGGACGGGGACGGACCGGTCACGACTC
CAGTCGCAGGAGCAGCCCTGGCCCAGGATGCTTTTCATAGGAAGGCAAGT
TCCCTGGACTCCGCAGTTCCCATCGCTCCACCTCCTCGCCAGGCCTGTTC
CTCCCTGGGTCCTGTCTTGAATGAGTCTAGACCTGTCGTTTGTGAAAGGC
ACAGGGTGGTGGTTTCCTATCCTCCTCAGAGTGAGGCAGAACTTGAACTT
AAAGAAGGAGATATTGTGTTTGTTCATAAAAAACGAGAGGATGGCTGGTT
CAAACGCACATTACAACGTAATGGGAAAACTGGCCTTTTCCCAGGAAGCT
TTGTGGAAAACATATGA
Human POSH Amino Acid Sequence

(SEQ ID NO: 2)

MDESALLDLLECPVCLERLDASAKVLPCQHTFCKRCLLGIVGSRNELRCP
ECRTLVGSGVEELPSNILLVRLLDGIKQRPWKPGPGGGSGTNCTNALRSQ
SSTVANCSSKDLQSSQGGQQPRVQSWSPPVRGIPQLPCAKALYNYEGKEP
GDLKFSKGDIIILRRQVDENWYHGEVNGIHGFFPTNFVQIIKPLPQPPPQ
CKALYDFEVKDKEADKDCLPFAKDDVLTVIRRVDENWAEGMLADKIGIFP
ISYVEFNSAAKQLIEWDKPPVPGVDAGECSSAAAQSSTAPKHSDTKKNTK
KRHSFTSLTMANKSSQASQNRHSMEISPPVLISSSNPTAAARISELSGLS
CSAPSQVHISTTGLIVTPPPSSPVTTGPSFTFPSDVPYQAALGTLNPPLP
PPPLLAATVLASTPPGATAAAAAAGMGPRPMAGSTDQIAHLRPQTRPSVY
VAIYPYTPRKEDELELRKGEMFLVFERCQDGWFKGTSMHTSKIGVFPGNY
VAPVTRAVTNASQAKVPMSTAGQTSRGVTMVSPSTAGGPAQKLQGNGVAG
SPSVVPAAVVSAAHIQTSPQAKVLLHMTGQMTVNQARNAVRTVAAHNQER
PTAAVTPIQVQNAAGLSPASVGLSHHSLASPQPAPLMPGSATHTAAISIS
RASAPLACAAAAPLTSPSITSASLEAEPSGRIVTVLPGLPTSPDSASSAC
GNSSATKPDKDSKKEKKGLLKLLSGASTKRKPRVSPPASPTLEVELGSAE
LPLQGAVGPELPPGGGHGRAGSCPVDGDGPVTTAVAGAALAQDAFHRKAS
SLDSAVPIAPPPRQACSSLGPVLNESRPVVCERHRVVVSYPPQSEAELEL
KEGDIVFVHKKREDGWFKGTLQRNGKTGLFPGSFVENI
Human POSH cDNA Sequence

(SEQ ID NO: 3)

CTGAGAGACACTGCGAGCGGCGAGCGCGGTGGGGCCGCATCTGCATCAGC
CGCCGCAGCCGCTGCGGGGCCGCGAACAAAGAGGAGGAGCCGAGGCGCGA
GAGCAAAGTCTGAAATGGATGTTACATGAGTCATTTTAAGGGATGCACAC
AACTATGAACATTTCTGAAGATTTTTTCTCAGTAAAGTAGATAAAGATGG
ATGAATCAGCCTTGTTCGATCTTTTGGAGTGTCCGGTGTGTCTAGAGCGC
CTTGATGCTTCTGCGAAGGTCTTGCCTTGCCAGCATACGTTTTGCAAGCG
ATGTTTGCTGGGGATCGTAGGTTCTCGAAATGAACTCAGATGTCCCGAGT
GCAGGACTCTTGTTGGCTCGGGTGTCGAGGAGCTTCCCAGTAACATCTTG
CTGGTCAGACTTCTGGATGGCATCAAACAGAGGCCTTGGAAACCTGGTCC
TGGTGGGGGAAGTGGGACCAACTGCACAAATGCATTAAGGTCTCAGAGCA
GCACTGTGGCTAATTGTAGCTCAAAAGATCTGCAGAGCTCCCAGGGCGGA
CAGCAGCCTCGGGTGCAATCCTGGAGCCCCCCAGTGAGGGGTATACCTCA
GTTACCATGTGCCAAAGCGTTATACAACTATGAAGGAAAAGACCCTGGAT
ACCTTAAATTCAGCAAAGGCGACATCATCATTTTGCGAAGACAAGTGGAT
GAAAATTGGTACCATGGGGAAGTCAATGGAATCCATGGCTTTTTCCCCAC
CAACTTTGTGCAGATTATTAAACCGTTACCTCAGCCCCCACCTCAGTGCA
AAGCACTTTATGACTTTGAAGTGAAAGACAAGGAAGCAGACAAAGATTGC
CTTCCATTTGCAAAGGATGATGTTCTGACTGTGATCCGAAGAGTGGATGA
AAACTGGGCTGAAGGAATGCTGGCAGACAAAATAGGAATATTTCCAATTT
CATATGTTGAGTTTAACTCGGCTGCTAAGCAGCTGATAGAATGGGATAAG
CCTCCTGTGCCAGGAGTTGATGCTGGAGAATGTTCCTCGGCAGCAGCCCA
GAGCAGCACTGCCCCAAAGCACTCCGACACCAAGAAGAACACCAAAAAGC
GGCACTCCTTCACTTCCCTCACTATGGCCAACAAGTCCTCCCAGGCATCC
CAGAACCGCCACTCCATGGAGATCAGCCCCCCTGTCCTCATCAGCTCCAG
CAACCCCACTGCTGCTGCACGGATCAGCGAGCTGTCTGGGCTCTCCTGCA
GTGCCCCTTCTCACGTTCATATAACTACCACCCGGTTAATTGTGACCCCG
CCCCCAAGCAGCCCAGTGACAACTGGCCCCTCGTTTACTTTCCCATCAGA
TGTTCCCTACCAAGCTGCCCTTGGAACTTTGAATCCTCCTCTTCCACCAC
CCCCTCTCCTGGCTGCCACTGTCCTTGCCTCCACACCACCAGGCGCCACC
GCCGCCGCTGCTGCTGCTGGAATGGGACCGAGGCCCATGGCAGGATCCAC
TGACCAGATTGCACATTTACGGCCGCAGACTCGCCCCAGTGTGTATGTTG
CTATATATCCATACACTCCTCGGAAAGAGGATGAACTAGAGCTGAGAAAA
GGGGAGATGTTTTTAGTGTTTGAGCGCTGCCAGGATGGCTGGTTCAAAGG
GACATCCATGCATACCAGCAACATAGGCGTTTTCCCTGGCAATTATGTGG
CACCAGTCACAAGGGCGGTGACAAATGCTTCCCAAGCTAAAGTCCCTATG
TCTACAGCTGGCCAGACAAGTCGGGGAGTGACCATGGTCAGTCCTTCCAC
GGCAGGAGGGCCTGCCCAGAAGCTCCAGGGAAATGGCGTGGCTGGGAGTC
CCAGTGTTGTCCCCGCAGCTGTGGTATCAGCAGCTCACATCCAGACAAGT
CCTCAGGCTAAGGTCTTGTTGCACATGACGGGGCAAATGACAGTCAACCA
GGCCCGCAATGCTGTGAGGACAGTTGCAGCGCACAACCAGGAACGCCCCA
CGGCAGCAGTGACACCCATCCAGGTACAGAATGCCGCCCCCCTCAGCCCT
GCATCTGTGGGCCTGTCCCATCACTCGCTGGCCTCCCCACAACCTGCGCC
TCTGATGCCAGGCTCAGCCACGCACACTGCTGCCATCAGTATCAGTCGAG
CCAGTGCCCCTCTGGCCTGTGCAGCAGCTGCTCCACTGACTTCCCCAAGC
ATCACCAGTGCTTCTCTGGAGGCTGAGCCCAGTGGCCGGATAGTGACCGT
TCTCCCTGGACTCCCCACATCTCCTGACAGTGCTTCATCAGCTTGTGGGA
ACAGTTCAGCAACCAAACCAGACAAGGATAGCAAAAAAGAAAAAAAGGGT
TTGTTGAAGTTGCTTTCTGGCGCCTCCACTAAACGGAAGCCCCGCGTGTC
TCCTCCAGCATCGCCCACCCTAGAAGTGGAGCTGGGCAGTGCAGAGCTTC
CTCTCCAGGGAGCGGTGGGGCCCGAACTGCCACCAGGAGGTGGCCATGGC
AGGGCAGGCTCCTGCCCTGTGGACGGGGACGGACCGGTCACGACTGCAGT
GGCAGGAGCACCCCTCGCCCAGGATGCTTTTCATAGGAAGGCAAGTTCCC
TGGACTCCGCAGTTCCCATCGCTCCACCTCCTCGCCACGCCTGTTCCTCC
CTGGGTCCTCTCTTGAATGAGTCTAGACCTGTCGTTTGTGAAAGGCACAG
GGTGGTGGTTTCCTATCCTCCTCAGAGTGAGGCAGAACTTGAACTTAAAG
AAGGAGATATTGTGTTTGTTCATAAAAAACGAGAGGATGGCTGGTTCAAA
GGCACATTACAACGTAATGGGAAAACTGGCCTTTTCCCAGGAAGCTTTGT
GGAAAACATATGAGGAGACTGACACTGAAGAAGCTTAAAATCACTTCACA
CAACAAAGTAGCACAAAGCAGTTTAACAGAAAGAGCACATTTGTGGACTT
CCAGATGGTCAGGAGATGAGCAAAGGATTGGTATGTGACTCTGATGCCCC
AGCACAGTTACCCCAGCGAGCAGAGTGAAGAAGATGTTTGTGTGGGTTTT
GTTAGTCTGGATTCGGATGTATAAGGTGTGCCTTGTACTGTCTGATTTAC
TACACAGAGAAACTTTTTTTTTTTTTTAAGATATATGACTAAAATGGACA
ATTGTTTACAAGGCTTAACTAATTTATTTGCTTTTTTAAACTTGAACTTT
TCGTATAATAGATACGTTCTTTGGATTATGATTTTAAGAAATTATTAATT
TATGAAATGATAGGTAAGGAGAAGCTGGATTATCTCCTGTTGAGAGCAAG
AGATTCGTTTTGACATAGAGTGAATGCATTTTCCCCTCTCCTCCTCCCTG
CTACCATTATATTTTGGGGTTATGTTTTGCTTCTTTAAGATAGAAATCCC
AGTTCTCTAATTTGGTTTTCTTCTTTGGGAAACCAAACATACAAATGAAT
CAGTATCAATTAGGGCCTGGGGTAGAGAGACAGAAACTTGAGAGAAGAGA
AGTTAGTGATTCCCTCTCTTTCTAGTTTGGTAGGAATCACCCTGAAGACC
TAGTCCTCAATTTAATTGTGTCGGTTTTTAATTTTCCTAGAATGAAGTGA
CTGAAACAATGAGAAAGAATACAGCACAACCCTTGAACAAAATGTATTTA
GAAATATATTTAGTTTTATAGCAGAAGCAGCTCAATTGTTTGGTTGGAAA
GTAGGGGAAATTGAAGTTGTAGTCACTGTCTGAGAATGGCTATGAAGCGT
CATTTCACATTTTACCCCAACTGACCTGCATGCCCAGGACACAAGTAAAA
CATTTGTGAGATAGTGGTGGTAAGTGATGCACTCGTGTTAAGTCAAAGGC
TATAAGAAACACTGTGAAAAGTTCATATTCATCCATTGTGATTCTTTCCC
CACGTCTTGCATGTATTACTGGATTCCCACAGTAATATAGACTGTGCATG
GTCTGTATATTTCATTGCGATTTCCTGTTAAGATGAGTTTGTACTCAGAA
TTGACCAATTCAGGAGGTGTAAAAATAAACAGTGTTCTCTTCTCTACCCC
AAAGCCACTACTGACCAAGGTCTCTTCAGTGCACTCGCTCCCTCTCTCGC
TAAGGCATGCATTAGCCACTACACAAGTCATTAGTGAAAGTGGTTTTTTA
TGTCCTCCCAGCAGACAGACATCAAGGATGAGTTAACCACGAGACTACTC
CTGTGACTGTGGACCTCTGGAAGGCTTGGTGGGAGTGAATTTGCCCACAC
CTTACAATTGTGGCAGGATCCAGAAGAGCCTGTCTTTTTATATCCATTCC
TTGATGTCATTGGCCTCTCCCACCGATTTCATTACGGTGCCACGCAGTCA
TGGATCTGGGTAGTCCGGAAAACAAAAGGAGGGAAGACAGCCTGGTAATG
AATAAGATCCTTACCACAGTTTTCTCATCGGAAATACATAATAAACCCTT
TCATCTTTTTTTTTTTCCTTTAAGAATTAAAACTGGGAAATAGAAACATG
AACTGAAAAGTCTTGCAATGACAAGAGGTTTCATGGTCTTAAAAAGATAC
TTTATATGGTTGAAGATGAAATCATTCCTAAATTAACCTTTTTTTTAAAA
AAAAACAATGTATATTATGTTCCTGTGTGTTGAATTTAAAAAAAAAAAAT
ACTTTACTTGGATATTCATGTAATATATAAAGGTTTGGTGAAATGAACTT
TAGTTAGGAAAAACCTCGCATCAGCTTTCATCTGTGTAAGTTGACACCAA
TGTGTCATAATATTCTTTATTTTGGGAAATTAGTGTATTTTATAAAAATT
TTAAAAAGAAAAAAGACTACTACAGGTTAAGATAATTTTTTTACCTGTCT
TTTCTCCATATTTTAAGCTATGTGATTGAAGTACCTCTGTTCATAGTTTC
CTGGTATAAAGTTGGTTAAAATTTCATCTCTTAATAGATCATTAGGTAAT
ATAATGTATGGGTTTTCTATTGGTTTTTTGCAGACAGTAGAGGGAGATTT
TGTAACAAGGGCTTGTTACACAGTGATATGGTAATGATAAAATTGCAATT
TATCACTCCTTTTCATGTTAATAATTTGAGGACTGGATAAAAGGTTTCAA
GATTAAAATTTGATGTTCAAACCTTTGT
5′ cDNA fragment of human POSH
(public gi: 10432611; SEQ ID NO: 4)
ctgagagacactgcgagcggcgagcgcggtggggccgcatctgcatcagc
cgccgcagccgctgcggggccgcgaacaaagaggaggagccgaggcgcga
gagcaaagtctgaaatggatgttacatgagtcattttaaggatgcacaca
actatgaacatttctgaagattttttctcagtaaagtagataaagatgga
tgaatcagccttgttggatcttttggagtgtccggtgtgtctagagcgcc
ttgatgcttctgcgaaggtcttgccttgccagcatacgttttgcaagcga
tgtttgctggggatcgtaggttctcgaaatgaactcagatgtcccgagtg
caggactcttgttggctcgggtgtcgaggagcttcccagtaacatcttgc
tggtcagacttctggatggcatcaaacagaggccttggaaacctggtcct
ggtgggggaagtgggaccaactgcacaaatgcattaaggtctcagagcag
cactgtggctaattgtagctcaaaagatctgcagagctcccagggcggac
agcagcctcgggtgcaatcctggagccccccagtgaggggtatacctcag
ttaccatgtgccaaagcgttatacaactatgaaggaaaagagcctggaga
ccttaaattcagcaaaggcgacatcatcattttgcgaagacaagtggatg
aaaattggtaccatggggaagtcaatggaatccatggctttttccccacc
aactttgtgcagattattaaaccgttacctcagcccccacctcagtgcaa
agcactttatgactttgaagtgaaagacaaggaagcagacaaagattgcc
ttccatttgcaaaggatgatgttctgactgtgatccgaagagtggatgaa
aactgggctgaaggaatgctggcagacaaaataggaatatttccaatttc
atatgttgagtttaactcggctgctaagcagctgatagaatgggataagc
ctcctgtgccaggagttgatgctggagaatgttcctcggcagcagcccag
agcagcactgccccaaagcactccgacaccaagaagaacaccaaaaagcg
gcactccttcacttccctcactatggccaacaagtcctcccaggcatccc
agaaccgccactccatggagatcagcccccctgtcctcatcagctccagc
aaccccactgctgctgcacggatcagcgagctgtctgggctctcctgcag
tgccccttctcaggttcatataagtaccaccgggttaattgtgaccccgc
ccccaagcagcccagtgacaactggcccctcgtttactttcccatcagat
gttccctaccaagctgcccttggaactttgaatcctcctcttccaccacc
ccctctcctggctgccactgtccttgcctccacaccaccaggcgccaccg
ccgccgctgctgctgctggaatgggaccgaggcccatggcaggatccact
gaccagattgcacatttacggccgcagactcgccccagtgtgtatgttgc
tatatatccatacactcctcggaaagaggatgaactagagctgagaaaag
gggagatgtttttagtgtttgagcgctgccaggatggctggttcaaaggg
acatccatgcataccagcaagataggggttttccctggcaattatgtggc
accagtcacaagggcggtgacaaatgcttcccaagctaaagtccctatgt
ctacagctggccagacaagtcggggagtgaccatggtcagtccttccacg
gcaggagggcctgcccagaagctccagggaaatggcgtggctgggagtcc
cagtgttgtccccgcagctgtggtatcagcagctcacatccagacaagtc
ctcaggctaaggtcttgttgcacatgacggggcaaatgacagtcaaccag
gcccgcaatgctgtgaggacagttgcagcgcacaaccaggaacgccccac
ggcagcagtgacacccatccaggtacagaatgccgccggcctcagccctg
catctgtgggcctgtcccatcactcgctggcctccccacaacctgcgcct
ctgatgccaggctcagccacgcacactgctgccatcagtatcagtcgagc
cagtgcccctctggcctgtgcagcagctgctccactgacttccccaagca
tcaccagtgcttctctggaggctgagcccagtggccggatagtgaccgtt
ctccctggactccccacatctcctgacagtgcttcatcagcttgtgggaa
cagttcagcaaccaaaccagacaaggatagc
N terminus protein fragment of hPOSH
(public gi: 10432612; SEQ ID NO: 5)
MDESALLDLLECPVCLERLDASAKVLPCQHTFCKRCLLGIVGSRNELRCP
ECRTLVGSGVEELPSNILLVRLLDGIKQRPWKPGPGGGSGTNCTNALRSQ
SSTVANCSSKDLQSSQGGQQPRVQSWSPPVRGIPQLPCAKALYNYEGKEP
GDLKFSKGDIIILRRQVDENWYHGEVNGIMGFFPTNFVQIIKPLPQPPPQ
CKALYDFEVKDKEADKDCLPFAKDDVLTVIRRVDENWAEGMLADKIGIFP
ISYVEFNSAAKQLIEWDKPPVPGVDACECSSAAAQSSTAPKHSDTKKNTK
KRHSFTSLTMANKSSQASQNRHSMEISPPVLISSSNPTAAARISELSGLS
CSAPSQVHISTTGLIVTPPPSSPVTTGPSFTFPSDVPYQAALGTLNPPLP
PPPLLAATVLASTPPGATAAAAAAGMGPRPMAGSTDQIAHLRPQTRPSVY
VAIYPYTPRKEDELELRKGENFLVFERCQDCWFKGTSMMTSKIGVFPGNY
VAPVTRAVTNASQAKVPMSTAGQTSRGVTMVSPSTACGPAQKLQGNGVAG
SPSVVPAAVVSAAHIQTSPQAKVLLHMTGQMTVNQARNAVRTVAAMNQER
PTAAVTPIQVQNAAGLSPASVGLSHHSLASPQPAPLMPGSATHTAAISIS
RASAPLACAAAAPLTSPSITSASLEAEPSGRIVTVLPGLPTSPDSASSAC
GNSSATKPDKDS
3′ mRNA fragment of hPOSH
(public gi: 7959248; SEQ ID NO: 6)
atttcatatgttgagtttaactcggctgctaagcagctgatagaatggga
taagcctcctgtgccaggagttgatgctggagaatgttcctcggcagcag
cccagagcagcactgccccaaagcactccgacaccaagaagaacaccaaa
aagcggcactccttcacttccctcactatggccaacaagtcctcccaggc
atcccagaaccgccactccatggagatcagcccccctgtcctcatcagct
ccagcaaccccactgctgctgcacggatcagcgagctgtctgggctctcc
tgcagtgccccttctcaggttcatataagtaccaccgggttaattgtgac
cccgcccccaagcagcccagtgacaactggcccctcgtttactttcccat
cagatgttccctaccaagctgcccttggaactttgaatcctcctcttcca
ccaccccctctcctggctgccactgtccttgcctccacaccaccaggcgc
caccgccgctgctgctgctgctggaatgggaccgaggcccatggcaggat
ccactgaccagattgcacatttacggccgcagactcgccccagtgtgtat
gttgctatatatccatacactcctcggaaagaggatgaactagagctgag
aaaaggggagatgtttttagtgtttgagcgctgccaggatggctggttca
aagggacatccatgcataccagcaagataggggttttccctggcaattat
gtggcaccagtcacaagggcggtgacaaatgcttcccaagctaaagtccc
tatgtctacagctggccagacaagtcggggagtgaccatggtcagtcctt
ccacggcaggagggcctgcccagaagctccagggaaatggcgtggctggg
agtcccagtgttgtccccgcagctgtggtatcagcagctcacatccagac
aagtcctcaggctaaggtcttgttgcacatgacggggcaaatgacagtca
accaggcccgcaatgctgtgaggacagttgcagcgcacaaccaggaacgc
cccacggcagcagtgacacccatccaggtacagaatgccgccggcctcag
ccctgcatctgtgggcctgtcccatcactcgctggcctccccacaacctg
cgcctctgatgccaggctcagccacgcacactgctgccatcagtatcagt
cgagccagtgcccctctggcctgtgcagcagctgctccactgacttcccc
aagcatcaccagtgcttctctggaggctgagcccagtggccggatagtga
ccgttctccctggactccccacatctcctgacagtgcttcatcagcttgt
gggaacagttcagcaaccaaaccagacaaggatagcaaaaaagaaaaaaa
gggtttgttgaagttgctttctggcgcctccactaaacggaagccccgcg
tgtctcctccagcatcgcccaccctagaagtggagctgggcagtgcagag
cttcctctccagggagcggtggggcccgaactgccaccaggaggtggcca
tggcagggcaggctcctgccctgtggacggggacggaccggtcacgactg
cagtggcaggagcagccctggcccaggatgcttttcataggaaggcaagt
tccctggactccgcagttcccatcgctccacctcctcgccaggcctgttc
ctccctgggtcctgtcttgaatgagtctagacctgtcgtttgtgaaaggc
acagggtggtggtttcctatcctcctcagagtgaggcagaacttgaactt
aaagaaggagatattgtgtttgttcataaaaaacgagaggatggctggtt
caaaggcacattacaacgtaatgggaaaactggccttttcccaggaagct
ttgtggaaaacatatgaggagactgacactgaagaagcttaaaatcactt
cacacaacaaagtagcacaaagcagtttaacagaaagagcacatttgtgg
acttccagatggtcaggagatgagcaaaggattggtatgtgactctgatg
ccccagcacagttaccccagcgagcagagtgaagaagatgtttgtgtggg
ttttgttagtctggattcggatgtataaggtgtgccttgtactgtctgat
ttactacacagagaaacttttttttttttttaagatatatgactaaaatg
gacaattgtttacaaggcttaactaatttatttgcttttttaaacttgaa
cttttcgtataatagatacgttctttggattatgattttaagaaattatt
aatttatgaaatgataggtaaggagaagctggattatctcctgttgagag
caagagattcgttttgacatagagtgaatgcattttcccctctcctcctc
cctgctaccattatattttggggttatgttttgcttctttaagatagaaa
tcccagttctctaatttggttttcttctttgggaaaccaaacatacaaat
gaatcagtatcaattagggcctggggtagagagacagaaacttgagagaa
gagaagttagtgattccctctctttctagtttggtaggaatcaccctgaa
gacctagtcctcaatttaattgtgtgggtttttaattttcctagaatgaa
gtgactgaaacaatgagaaagaatacagcacaacccttgaacaaaatgta
tttagaaatatatttagttttatagcagaagcagctcaattgtttggttg
gaaagtaggggaaattgaagttgtagtcactgtctgagaatggctatgaa
gcgtcatttcacattttaccccaactgacctgcatgcccaggacacaagt
aaaacatttgtgagatagtggtggtaagtgatgcactcgtgttaagtcaa
aggctataagaaacactgtgaaaagttcatattcatccattgtgattctt
tccccacgtcttgcatgtattactggattcccacagtaatatagactgtg
catggtgtgtatatttcattgcgatttcctgttaagatgagtttgtactc
agaattgaccaattcaggaggtgtaaaaataaacagtgttctcttctcta
ccccaaagccactactgaccaaggtctcttcagtgcactcgctccctctc
tggctaaggcatgcattagccactacacaagtcattagtgaaagtggtct
tttatgtcctcccagcagacagacatcaaggatgagttaaccaggagact
actcctgtgactgtggagctctggaaggcttggtgggagtgaatttgccc
acaccttacaattgtggcaggatccagaagagcctgtctttttatatcca
ttccttgatgtcattggcctctcccaccgatttcattacggtgccacgca
gtcatggatctgggtagtccggaaaacaaaaggagggaagacagcctggt
aatgaataagatccttaccacagttttctcatgggaaatacataataaac
cctttcatctttttttttttcctttaagaattaaaactgggaaatagaaa
catgaactgaaaagtcttgcaatgacaagaggtttcatggtcttaaaaag
atactttatatggttgaagatgaaatcattcctaaattaacctttttttt
aaaaaaaaacaatgtatattatgttcctgtgtgttgaatttaaaaaaaaa
aaatactttacttggatattcatgtaatatataaaggtttggtgaaatga
actttagttaggaaaaagctggcatcagctttcatctgtgtaagttgaca
ccaatgtgtcataatattctttattttgggaaattagtgtattttataaa
aattttaaaaagaaaaaagactactacaggttaagataatttttttacct
gtcttttctccatattttaagctatgtgattgaagtacctctgttcatag
tttcctggtataaagttggttaaaatttcatctgttaatagatcattagg
taatataatgtatgggttttctattggttttttgcagacagtagagggag
attttgtaacaagggcttgttacacagtgatatggtaatgataaaattgc
aatttatcactccttttcatgttaataatttgaggactggataaaaggtt
tcaagattaaaatttgatgttcaaacctttgt
C terminus protein fragment of hPOSH
(public gi: 7959249; SEQ ID NO: 7)
ISYVEFNSAAKQLIEWDKPPVPGVDACECSSAAAQSSTAPKHSDTKKNTK
KRHSFTSLTMANKSSQASQNRHSMEISPPVLISSSNPTAAARISELSGLS
CSAPSQVHISTTGLIVTPPPSSPVTTGPSFTFPSDVPYQAALGTLNPPLP
PPPLLAATVLASTPPGATAAAAAAGMGPRPMAGSTDQIAHLRPQTRPSVY
VAIYPYTPRKEDELELRKGEMFLVFERCQDGWFKGTSMFTSKIGVFPGNY
VAPVTRAVTNASQAKVPMSTAGQTSRGVTMVSPSTAGGPAQKLQGNCVAG
SPSVVPAAVVSAAHIQTSPQAKVLLMNTGQMTVNQARNAVRTVAAHNQER
PTAAVTPIQVQNAAGLSPASVCLSMHSLASPQPAPLMPGSATHTAAISIS
RASAPLACAAAAPLTSPSITSASLEAEPSGRIVTVLPGLPTSPDSASSAC
GNSSATKPDKDSKKEKKGLLKLLSGASTKRKPRVSPPASPTLEVELGSAE
LPLQGAVGPELPPGGGHGRAGSCPVDGDGPVTTAVACAALAQDAFHRKAS
SLDSAVPIAPPPRQACSSLGPVLNESRPVVCERHRVVVSYPPQSEAELEL
KEGDIVFVHKKREDGWFKGTLQRNGKTGLFPGSFVENI
Mouse POSH mRNA sequence
(public gi: 10946921; SEQ ID NO: 8)
GGGCAGCGGGCTCGGCGGGGCTGCATCTACCAGCGCTGCGGGGCCGCGAA
CAAAGGCGAGCAGCGGAGGCGCGAGAGCAAAGTCTGAAATGGATGTTACA
TGAATCACTTTAAGGGCTGCGCACAACTATGAACGTTCTGAAGCCGTTTT
CTCACTAAAGTCACTCAAGATGGATGAGTCTGCCTTGTTGGACCTTCTGG
AGTGCCCTGTGTGTCTAGAACGCCTGGATGCTTCCGCAAAGGTCTTACCC
TGCCAGCATACCTTTTGCAAACGCTGTTTGCTGGGGATTGTGGGTTCCCG
GAATGAACTCAGATGTCCCGAATGCCGGACTCTTGTTGGCTCTCGGGTCG
ACGAGCTCCCCAGTAACATCCTACTGGTCAGACTTCTGGATGGCATCAAG
CAGAGGCCTTGGAAACCCGGCCCTGGTGGGGGCGGCGGGACCACCTGCAC
AAACACATTAAGGGCGCAGGGCAGCACTGTGGTTAATTGTGGCTCGAAAG
ATCTGCAGAGCTCCCAGTGTGGACAGCAGCCTCGGGTCCAAGCCTGGAGC
CCCCCAGTGAGGGGAATACCTCAGTTACCGTGTGCCAAAGCATTATATAA
CTACGAAGGAAAAGAGCCCGGAGACCTTAAGTTCAGCAAAGGCGACACCA
TCATTCTGCGCCGACAGGTGGATGAGAATTGGTACCACGGGGAAGTCAGC
CGGGTCCACGGCTTTTTCCCCACTAACTTCGTGCAGATCATCAAACCTTT
ACCTCAGCCCCCGCCTCAGTGCAAAGCACTTTACGACTTTGAAGTGAAAG
ACAAGGAAGCTGACAAAGATTGCCTTCCCTTCGCAAAGGACCACGTACTG
ACCGTGATCCGCAGAGTGGATGAAAACTGGGCTGAAGGAATGCTGGCAGA
TAAAATAGGAATATTTCCAATTTCATACGTGGAGTTTAACTCACCTGCCA
AGCAGCTGATAGAGTGGGATAAGCCTCCCGTGCCAGGAGTGGACACGGCA
GAATCCCCCTCAGCGACGGCGCAGAGCACCTCTGCCTCAAAGCACCCCGA
CACCAAGAAGAACACCAGGAAGCGACACTCCTTCACCTCCCTCACCATGG
CCAACAAGTCTTCCCAGGGGTCCCAGAACCGCCACTCCATGGAGATCAGC
CCTCCTGTGCTCATCAGTTCCAGCAACCCCACAGCCGCAGCCCGCATCAG
CGAACTGTCCGGGCTCTCCTGCAGCGCCCCGTCTCAGGTCCATATAAGCA
CCACTGGGTTAATTGTGACCCCACCCCCTAGCAGCCCGGTGACAACTCGC
CCTGCGTTCACGTTCCCTTCAGATGTCCCCTACCAAGCTGCCCTTGGAAG
TATGAATCCTCCACTTCCCCCACCCCCTCTCCTGGCGGCCACCGTACTCG
CCTCCACCCCGTCAGGCGCTACTGCTGCTGTTGCTGCTGCTGCTGCCGCC
GCCGCCGCTGCTGGAATGGGACCCAGGCCTGTGATGGGGTCCTCTGAACA
GATTGCACATTTACGGCCTCAGACTCGTCCCAGTGTATATGTTGCTATAT
ATCCGTACACTCCCCGGAAGGAAGACGAACTGGAGCTGAGGAAAGGGGAG
ATGTTTTTGGTGTTTGAGCGTTGCCAGGACGGCTGGTACAAAGGGACATC
GATGCATACCAGCAAGATAGGCGTTTTCCCTGGCAACTATGTGGCGCCCG
TCACAAGGGCGGTGACGAATGCCTCCCAAGCTAAAGTCTCTATGTCTACT
GCGGGTCAGGCAAGTCGCGGGGTGACCATGGTCAGCCCTTCCACTGCAGG
AGGACCTACACAGAAGCCCCAAGGAAACGGCGTGGCCGGAAATCCCAGCG
TCGTCCCCACGGCTGTGGTGTCAGCAGCTCATATCCAGACAAGTCCTCAG
GCTAAGGTCCTGCTGCACATGTCTGGGCAGATGACAGTCAATCAGGCCCG
CAATGCTGTGAGGACAGTTGCAGCACATAGCCAGGAACGCCCCACAGCAG
CAGTGACTCCCATCCAGGTCCAGAATGCCGCCTGCCTTGGTCCTGCATCC
GTGGGCCTGCCCCATCATTCTCTGGCCTCCCAACCTCTGCCTCCAATGGC
GGGTCCTGCTGCCCACGGTGCTGCCGTCAGCATCAGTCGAACCAATGCCC
CCATGGCCTGCGCTGCAGGGGCTTCTCTGGCCTCCCCAAATATCACCAGT
GCCATGTTGGAGACAGAGCCCAGTGGTCGCACAGTGACCATCCTCCCTGG
ACTCCCCACATCTCCAGAGAGTGCTGCATCAGCGTGTGGGAACAGTTCAG
CTGGGAAACCAGACAAGGACAGTAAGAAAGAAAAAAACGGCCTACTGAAG
CTGCTTTCTGGTGCCTCCACCAAACGCAAGCCCCGAGTCTCCCCTCCAGC
ATCACCTACCCTGGATGTGGAGCTGGGTGCTGGGGAGGCTCCCTTGCAGG
GAGCAGTAGGTCCTGAGCTGCCGCTAGGGGGCAGCCACGGCAGAGTCGGG
TCATGCCCCACAGATGGTGATGGTCCAGTGGCCGCTGGAACAGCAGCCCT
AGCCCAGGATGCCTTCCACCGCAAGACAAGCTCCCTGGACTCCCCAGTGC
CCATTGCTCCACCACCTCGCCAGGCCTGCTCCTCCCTGGGCCCAGTCATG
AATGACGCCCGGCCTGTTGTTTGTGAAAGGCACAGGGTGGTGGTTTCCTA
CCCTCCTCAGAGTGAGGCCGAACTTGAACTCAAGGAAGGAGATATTGTGT
TTGTTCATAAGAAACGAGAGGACGGCTGGTTCAAAGGCACGTTACAGAGG
AATGGGAAGACTGGCCTTTTCCCAGGGAGCTTTGTGGAAAACATCTGACA
AGACGGGACACGGAGAAAGCTTATCATCACACCACGTGTGACTAAAGAGC
ACAAAGCAGTTTCATAGAAAGAGCACATCTGTGGACTTCCAGATCTTCAA
GAACCGAGCAGAAGATGGGCACCTGACTCCAGAGCCCCGGCCTGGTTACC
CCAGGGGCAGAGGGAAGGAGGACACACCTGTGTGGGTTCCGTCTCTCTGG
GTTCTGATGTGTAAAGTGTGCCTTGTAATGTCTAATGGACTTTACAGATA
AATGTCTTTTTTTTTTTAAGATGTATAACTAAAATCGACAATTGTTTACA
AGGCTTAACTAATTTATTTGCTTTTTTAAAACTTGAACTTTCTTGTAATA
GCAAAT
Mouse POSH Protein sequence
(Public gi: 10946922; SEQ ID NO: 9)
MDESALLDLLECPVCLERLDASAKVLPCQHTFCKRCLLGIVGSRNELRCP
ECRTLVGSGVDELPSNILLVRLLDGIKQRPWKPGPGGGGGTTCTNTLRAQ
GSTVVNCGSKDLQSSQCGQQPRVOAWSPPVRGIPQLPCAKALYNYEGKEP
GDLKFSKGDTIILRRQVDENWYMGEVSGVHGFFPTNFVQIIKPLPQPPPQ
CKALYDFEVKDKEADKDCLPFAKDDVLTVIRRVDENWAEGMLADKIGIFP
ISYVEFNSAAKQLIEWDKPPVPCVDTAECPSATAQSTSASKHPDTKKNTR
KRHSFTSLTMANKSSQGSQNRHSMEISPPVLISSSNPTAAARISELSGLS
CSAPSQVNISTTGLIVTPPPSSPVTTGPAFTFPSDVPYQAALGSMNPPLP
PPPLLAATVLASTPSGATAAVAAAAAAAAAAGMGPRPVMGSSEQIAHLRP
QTRPSVYVAIYPYTPRKEDELELRKGEMFLVFERCQDGWYKGTSMMTSKI
GVFPGNYVAPVTRAVTNASQAKVSMSTAGQASRGVTMVSPSTAGGPTQKP
QGNGVAGNPSVVPTAVVSAAHIQTSPQAKVLLHNSGQMTVNQARNAVRTV
AAHSQERPTAAVTPIQVQNAACLGPASVGLPHHSLASQPLPPMAGPAAHG
AAVSISRTNAPMACAAGASLASPNMTSANLETEPSGRTVTILPGLPTSPE
SAASACGNSSAGKPDKDSKKEKKGLLKLLSGASTKRKPRVSPPASPTLDV
ELGAGEAPLQGAVGPELPLGGSHGRVGSCPTDCDGPVAAGTAALAQDAFH
RKTSSLDSAVPIAPPPRQACSSLGPVMNEARPVVCERHRVVVSYPPQSEA
ELELKEGDIVFVHKKREDGWFKGTLQRNGKTGLFPGSFVENI
Drosophila melanogaster POSH mRNA sequence
(public gi: 17737480; SEQ ID NO: 10)
CATTTGTATCCGCTTGGCCACGAGCTTTCGCTGCACTTGGCAAACTTAAT
AAATTAAACATTGAATCCTGCCTATTGCAACGATAATATAATCTGATTTA
GTGCATTAAGAACGACAAGTAGCGATTATAATAGTAGATTTTAGCATTTG
AGCTAAATTTATTTCCCAACCGCGTCTTGGGATTGCGTATGCGTGAGCCA
GTACCTGCATGTGTGTGTGTTTTGGAATGTCGCCCTGCACGAAATTCAAA
TAGTGACCATCCTTGAGATTTTGCATACTGGCAAGATGGACGAGCACACG
TTAAACGACCTGTTGGAGTGCTCCGTGTGTCTTGAGCGACTGGACACCAC
ATCGAAGGTGCTGCCATGCCAGCACACCTTCTGCCGCAAATGCTTGCAGG
ACATTGTGGCCAGTCAGCACAAGTTGCGATGCCCGGAGTGCCGCATCCTG
GTCTCTTGCAAAATTGATGAGCTGCCTCCAAACGTCTTGCTGATGCGAAT
CTTAGAAGGCATGAAACAAAATGCAGCAGCTGGCAAAGGAGAAGAAAACG
GAGAGGAGACTGAAACACAGCCGGAAAGGGCCAAACCTCAGCCGCCAGCG
GAATCAGTGGCCCCGCCTGACAACCAACTACTCCAGCTGCAGTCACATCA
GCAATCTCATCAGCCGGCTCGTCACAAGCAACGTCGATTTCTACTCCCCC
ACGCCTATGCCCTCTTTGACTTCGCCTCCGGTGAAGCCACCGATCTAAAG
TTCAAGAAAGGGGATCTGATACTGATCAAGCATCGCATCGACAACAACTG
GTTTGTGGGTCAAGCGAATGGTCAGGAGGGCACATTTCCCATCAACTACG
TCAACGTATCGGTTCCGCTGCCCATGCCCCAGTGCATTGCCATGTATGAC
TTTAAGATGGGGCCCAACGACGAGGAGGGATGCCTCGAATTTAAGAAAAG
CACTGTAATACAGGTAATGCGCCGAGTTGATCATAATTGCGCAGAAGGAC
GAATTGGCCAGACCATCGGAATCTTTCCAATAGCATTCGTTGAGCTGAAT
GCAGCGGCCAAAAAGCTGTTGGACAGCGGGCTACACACCCATCCATTCTG
CCATCCACCGAAGCAACAGGGGCACCGGGCCCTTCCTCCGGTTCCAGTTA
TTGATCCCACGGTGGTCACGGAATCCAGTTCGGGATCCTCCAATTCCACG
CCGGGCAGCAGCAATTCAAGCTCCACATCCAGCTCGAATAACTGCAGTCC
GAATCACCAAATCTCACTGCCGAATACCCCCCAACATGTAGTAGCTTCCG
GATCGGCGTCTGTTCGTTTCCGTGACAAGGGAGCAAACGAGAAACGCCAC
TCACTAAATGCTTTGCTGGGAGGAGGAGCTCCATTAAGTCTGCTGCAGAC
CAACCGCCATTCGGCTGAAATTCTTAGCCTGCCCCATGAACTAAGCCGCT
TGGAAGTTTCCAGCTCAACAGCTCTAAAACCCACGTCAGCCCCACAGACA
TCGCGTGTACTTAAGACCACTGTTCAGCAGCAGATGCAACCGAATTTACC
CTGGGGATACTTAGCCCTGTTCCCATACAAACCACGCCAAACGGATGAGC
TGGAATTAAAAAAGGGTTGTGTTTACATTGTGACCGAACGATGTGTGGAC
GGTTGGTTCAAGGGAAAAAACTGGTTGGACATCACTGGAGTGTTCCCGGG
CAACTACCTGACGCCCCTGCGCGCCCGCGACCAGCAGCAGTTAATGCATC
AATGGAAATATGTTCCCCAAAATGCAGACGCCCAGATGGCACAAGTACAG
CAGCATCCAGTTGCACCAGATGTGCGACTCAACAACATGCTGTCCATGCA
ACCGCCTGATTTGCCACCTCGTCAGCAGCAGGCTACCGCCACGACCACCA
GTTGCTCTGTGTGGTCCAAACCAGTGGAGGCGCTGTTCAGCAGAAAATCG
GAGCCCAAGCCTGAAACTGCCACAGCTTCGACTACGAGCACCAGTTCCTC
TGGAGCAGTGGGACTTATGAGGAGATTAACTCACATGAAAACACGCTCCA
AATCTCCGGGAGCGTCCTTGCAGCAAGTTCCGAAAGAAGCTATTAGCACA
AATGTGGAATTTACAACAAACCCATCAGCTAAATTGCATCCAGTACATGT
AAGATCCGGCTCGTGCCCCAGTCACCTGCAGCACAGTCAACCGCTCAATG
AAACTCCAGCAGCCAAGACAGCGOCACAACAACAGCAGTTCCTACCCAAG
CAGCTCCCTTCCGCTTCTACGAACAGCGTTTCGTACGGATCGCAACGCGT
GAAAGGAAGCAAGGAACGTCCTCACTTGATTTGCGCGAGACAATCATTAG
ATGCAGCTACATTTCGCAGTATGTACAACAATGCCGCGTCGCCGCCGCCA
CCTACTACTTCCGTGGCCCCACCTGTCTACGCCGGCGGTCAGCAACAGGT
GATTCCTGGAGGTGGAGCGCAATCCCAGTTGCATGCCAATATGATTATTG
CACCCAGCCATCGGAAGTCGCACAGCCTAGATGCGAGTCATGTGCTGAGT
CCCAGCAGCAATATGATCACGGAGGCGGCCATTAAGGCCAGCGCCACCAC
TAAGTCTCCTTACTGCACGAGGGAAAGTCGATTCCGCTGCATTGTGCCGT
ATCCACCAAACAGTGACATTGAACTAGAGCTACATTTGGGCGACATTATC
TACGTCCAGCGGAAGCAGAAGAACGGCTGGTATAAGGGCACCCATGCCCG
TACCCACAAAACCGGGCTGTTCCCCGCCTCCTTTGTTGAACCGGATTGTT
AGGAAAGTTATGGTTCAAACTAGAATTTATTAAGCGAAATTCCAAATTAC
TTGTCTAAAAGGATTCAATCGTCGGTCTATTCGGGCTTCCAAATACGCAA
TCTCATATTTCTCTTTTCAAAAAAGAAACCGTTTTGTACTCTTCCAATCG
AATGGGCAGCTCGCCGTTGTACTTTTTTATACAATGCTTGATCAAAATAG
GCTAGCCATGTAAGACTTAGGGAACAGTTACTTAAGCCTTAGCGATTAGT
TAGCTAGAGAAATAATCTAACCGATCCTTGTGCCCTCTACAAATGTTATT
TGTAATATACGATACTCAGTAATAAAAAAAAAAAAAAAAAAAAAAAAAA
Drosophila melanogaster POSH protein sequence
(public gi: 17737481; SEQ ID NO: 11)
MDEHTLNDLLECSVCLERLDTTSKVLPCQHTFCRKCLQDIVASQHKLRCP
ECRILVSCKIDELPPNVLLMRILEGMKQNAAAGKGEEKGEETETQPERAK
POPPAESVAPPDNQLLQLQSHQQSHQPARHKQRRPLLPHAYALFDFASGE
ATDLKFKKGDLILIKHRIDNNWFVGQANGQEGTFPINYVKVSVPLPMPQC
IAMYDFKMGPNDEEGCLEFKKSTVIQVMRRVDHNWAEGRIGQTIGIPPIA
FVELNAAAKKLLDSGLMTHPFCMPPKQQGQRALPPVPVIDPTVVTESSSG
SSNSTPGSSNSSSTSSSNNCSPNHQISLPNTPQHVVASGSASVRFRDKGA
KEKRHSLNALLGGGAPLSLLQTNRMSAEILSLPHELSRLEVSSSTALKPT
SAPQTSRVLKTTVQQQMQPNLPWGYLALFPYKPRQTDELELKKGCVYIVT
ERCVDGWFKGKNWLDITGVFPGNYLTPLRARDQQQLMHQWKYVPQNADAQ
MAQVQQHPVAPDVRLNNNLSMQPPDLPPRQQQATATTTSCSVWSKPVEAL
FSRKSEPKPETATASTTSSSSSGAVGLMRRLTHMKTRSKSPGASLQQVPK
EAISTNVEFTTNPSAKLHPVHVRSGSCPSQLQHSQPLNETPAAKTAAQQQ
QFLPKQLPSASTNSVSYGSQRVKGSKERPHLICARQSLDAATFRSMYNNA
ASPPPPTTSVAPAVYAGGQQQVIPGGGAQSQLHANMIIAPSHRKSHSLDA
SMVLSPSSNMITEAAIKASATTKSPYCTRESRFRCIVPYPPNSDIELELH
LGDIIYVQRKQKNGWYKGTMARTHKTGLFPASFVEPDC

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for treating an infectious disease, the method comprising, administering to a subject an agent that modulates a TGN-associated protein of the host cell, thereby inhibiting processing of a protein of an infectious organism.

2. The method of claim 1, wherein the agent inhibits POSH.

3. The method of claim 2, wherein the agent is a small molecule that inhibits POSH.

4. The method of claim 3, wherein the small molecule is selected from the group consisting of:

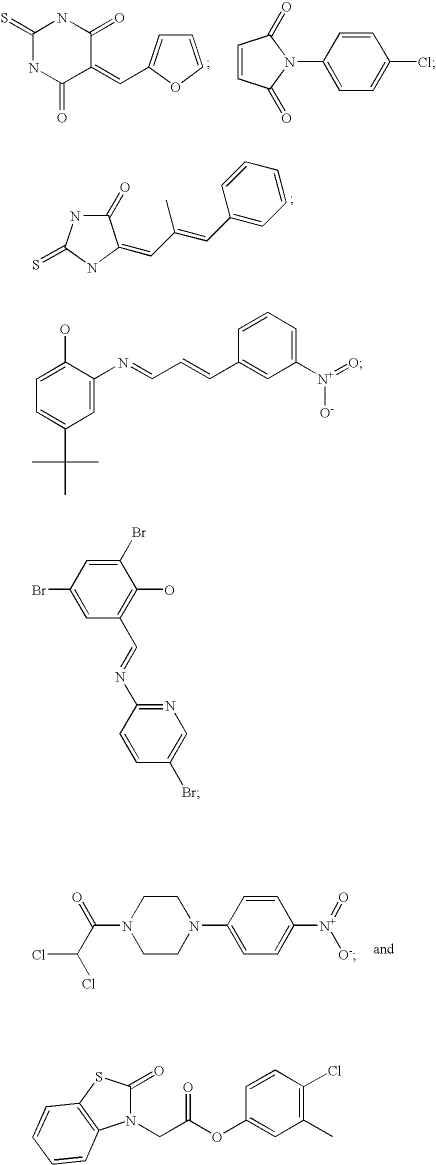

5. The method of claim 2, wherein the agent is a nucleic acid that hybridizes to a POSH mRNA and inhibits the expression of POSH protein.

6. The method of claim 2, wherein the subject has an infectious disease caused by a virus selected from the group consisting of: wV and West Nile Virus.

7. The method of claim 2, wherein the agent inhibits POSH ubiquitin ligase activity.

8. The method of claim 1, wherein the agent inhibits a protein selected from the group consisting of: Cbl-b, HERPUD1, GOCAP1, GOSR2, a PKA subunit, DDEF1, ARHV (CHP), SPG20 (spartin), CENTB1, dynaminII, and RALA.

9. A method for inhibiting aberrant processing of a protein associated with a disorder, the method comprising, administering an agent that modulates a polypeptide selected from the group consisting of: POSH, a POSH-pathway polypeptide, a POSH-AP and a POSH binding protein.

10. The method of claim 9, wherein the agent is administered to a subject having cancer.

11. The method of claim 9, wherein the agent is administered to a subject having an immunological disorder.

12. The method of claim 9, wherein the agent is administered to a subject having a neurological disorder.

13. The method of claim 9, wherein the disorder is Alzheimer's disease and the aberrantly processed protein is a beta-amyloid precursor protein.

14. A method of claim 9, wherein the agents inhibits a protein selected from the group consisting of: POSH and HERPUD1.

15. The method of claim 9, wherein the agent modulates the activity of a polypeptide selected from the group consisting of: Cbl-b, HERPUD1, GOCAP1, GOSR2, a PKA subunit, DDEF1, ARHV (CHP), SPG20 (spartin), CENTB1, dynaminII, and RALA.

16. The method of claim 9, wherein the secretion of a myristylated protein is inhibited.

17. A method for treating cancer, the method comprising administering an agent that inhibits POSH.

18. The method of claim 17, wherein the cancer is selected from among: thyroid carcinoma, liver cancer, hepatocellular cancer, lung cancer, cervical cancer, colorectal cancer, ovarian cancer, renal cell carcinoma, lymphoma, osteosarcoma, prostate cancer, liposarcoma, leukemia, breast carcinoma, and breast adeno-carcinoma.

19. A method of modulating the activity or localization of a TGN-associated protein comprising modulating the activity of one or more of the polypeptides selected from the group consisting of POSH, a POSH-pathway polypeptide, a POSH-AP and a POSH binding protein.

20. The method of claim 19, wherein the TGN-associated protein is a ubiquitin ligase.

21. The method of claim 19, wherein the TGN associated protein comprises a TGN-localization domain.

22. The method of claim 21, wherein the TGN-localization domain is selected from the group consisting of: a tyrosine based motif; acidic amino acid cluster; a casein kinase II phosphorylation site; VHS domain; GAT domain; a gamma ear domain; GRIP domain; ENTH domain; cysteine rich domain; and a granin motif.


SEQUENCE LISTING (175 sequences)

SEQ ID NO: 1 - DNA, 2667 bp, Homo sapiens
atggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatga

SEQ ID NO: 2 - protein, 888 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 3 - DNA, 5128 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 4 - DNA, 2331 bp, Homo sapiens
ctgagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaaggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgccgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagc

SEQ ID NO: 5 - protein, 712 aa, Homo sapiens
MDESALLDLLECPVCLERLDASAKVLPCQHTFCKRCLLGIVGSRNELRCPECRTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDS

SEQ ID NO: 6 - DNA, 4182 bp, Homo sapiens
atttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagagtgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatatggttgaagatgaaatcattcctaaattaaccttttttttaaaaaaaaacaatgtatattatgttcctgtgtgttgaatttaaaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaacctttgt

SEQ ID NO: 7 - protein, 638 aa, Homo sapiens
ISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 8 - DNA, 3206 bp, Mouse
gggcagcgggctcggcggggctgcatctaccagcgctgcggggccgcgaacaaaggcgagcagcggaggcgcgagagcaaagtctgaaatggatgttacatgaatcactttaagggctgcgcacaactatgaacgttctgaagccgttttctcactaaagtcactcaagatggatgagtctgccttgttggaccttctggagtgccctgtgtgtctagaacgcctggatgcttccgcaaaggtcttaccctgccagcataccttttgcaaacgctgtttgctggggattgtgggttcccggaatgaactcagatgtcccgaatgccggactcttgttggctctggggtcgacgagctccccagtaacatcctactggtcagacttctggatggcatcaagcagaggccttggaaacccggccctggtgggggcggcgggaccacctgcacaaacacattaagggcgcagggcagcactgtggttaattgtggctcgaaagatctgcagagctcccagtgtggacagcagcctcgggtgcaagcctggagccccccagtgaggggaatacctcagttaccgtgtgccaaagcattatataactacgaaggaaaagagcccggagaccttaagttcagcaaaggcgacaccatcattctgcgccgacaggtggatgagaattggtaccacggggaagtcagcggggtccacggctttttccccactaacttcgtgcagatcatcaaacctttacctcagcccccgcctcagtgcaaagcactttacgactttgaagtgaaagacaaggaagctgacaaagattgccttcccttcgcaaaggacgacgtactgaccgtgatccgcagagtggatgaaaactgggctgaaggaatgctggcagataaaataggaatatttccaatttcatacgtggagtttaactcagctgccaagcagctgatagagtgggataagcctcccgtgccaggagtggacacggcagaatgcccctcagcgacggcgcagagcacctctgcctcaaagcaccccgacaccaagaagaacaccaggaagcgacactccttcacctccctcaccatggccaacaagtcttcccaggggtcccagaaccgccactccatggagatcagccctcctgtgctcatcagttccagcaaccccacagccgcagcccgcatcagcgaactgtccgggctctcctgcagcgccccgtctcaggtccatataagcaccactgggttaattgtgaccccaccccctagcagcccggtgacaactggccctgcgttcacgttcccttcagatgtcccctaccaagctgcccttggaagtatgaatcctccacttcccccaccccctctcctggcggccaccgtactcgcctccaccccgtcaggcgctactgctgctgttgctgctgctgctgccgccgccgccgctgctggaatgggacccaggcctgtgatggggtcctctgaacagattgcacatttacggcctcagactcgtcccagtgtatatgttgctatatatccgtacactccccggaaggaagacgaactggagctgaggaaaggggagatgtttttggtgtttgagcgttgccaggacggctggtacaaagggacatcgatgcataccagcaagataggcgttttccctggcaactatgtggcgcccgtcacaagggcggtgacgaatgcctcccaagctaaagtctctatgtctactgcgggtcaggcaagtcgcggggtgaccatggtcagcccttccactgcaggaggacctacacagaagccccaaggaaacggcgtggccggaaatcccagcgtcgtccccacggctgtggtgtcagcagctcatatccagacaagtcctcaggctaaggtcctgctgcacatgtctgggcagatgacagtcaatcaggcccgcaatgctgtgaggacagttgcagcacatagccaggaacgccccacagcagcagtgactcccatccaggtccagaatgccgcctgccttggtcctgcatccgtgggcctgccccatcattctctggcctcccaacctctgcctccaatggcgggtcctgctgcccacggtgctgccgtcagcatcagtcgaaccaatgcccccatggcctgcgctgcaggggcttctctggcctccccaaatatgaccagtgccatgttggagacagagcccagtggtcgcacagtgaccatcctccctggactccccacatctccagagagtgctgcatcagcgtgtgggaacagttcagctgggaaaccagacaaggacagtaagaaagaaaaaaagggcctactgaagctgctttctggtgcctccaccaaacgcaagccccgagtctcccctccagcatcacctaccctggatgtggagctgggtgctggggaggctcccttgcagggagcagtaggtcctgagctgccgctagggggcagccacggcagagtggggtcatgccccacagatggtgatggtccagtggccgctggaacagcagccctagcccaggatgccttccaccgcaagacaagctccctggactccgcagtgcccattgctccaccacctcgccaggcctgctcctccctgggcccagtcatgaatgaggcccggcctgttgtttgtgaaaggcacagggtggtggtttcctaccctcctcagagtgaggccgaacttgaactcaaggaaggagatattgtgtttgttcataagaaacgagaggacggctggttcaaaggcacgttacagaggaatgggaagactggccttttcccagggagctttgtggaaaacatctgagaagacgggacacggagaaagcttatcatcacaccacgtgtgactaaagagcacaaagcagtttcatagaaagagcacatctgtggacttccagatcttcaagaaccgagcagaagatgggcacctgactccagagccccggcctggttaccccaggggcagagggaaggaggacacacctgtgtgggttccgtctctctgggttctgatgtgtaaagtgtgccttgtaatgtctaatggactttacagataaatgtctttttttttttaagatgtataactaaaatggacaattgtttacaaggcttaactaatttatttgcttttttaaaacttgaactttcttgtaatagcaaat

SEQ ID NO: 9 - protein, 892 aa, Mouse
MDESALLDLLECPVCLERLDASAKVLPCQHTFCKRCLLGIVGSRNELRCPECRTLVGSGVDELPSNILLVRLLDGIKQRPWKPGPGGGGGTTCTNTLRAQGSTVVNCGSKDLQSSQCGQQPRVQAWSPPVRGIPQLPCAKALYNYEGKEPGDLKFSKGDTIILRRQVDENWYHGEVSGVHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDTAECPSATAQSTSASKHPDTKKNTRKRHSFTSLTMANKSSQGSQNRHSMEISPPVLISSSNPTAAARISELSGLSCSAPSQVHISTTGLIVTPPPSSPVTTGPAFTFPSDVPYQAALGSMNPPLPPPPLLAATVLASTPSGATAAVAAAAAAAAAAGMGPRPVMGSSEQIAHLRPQTRPSVYVAIYPYTPRKEDELELRKGEMFLVFERCQDGWYKGTSMHTSKIGVFPGNYVAPVTRAVTNASQAKVSMSTAGQASRGVTMVSPSTAGGPTQKPQGNGVAGNPSVVPTAVVSAAHIQTSPQAKVLLHMSGQMTVNQARNAVRTVAAHSQERPTAAVTPIQVQNAACLGPASVGLPHHSLASQPLPPMAGPAAHGAAVSISRTNAPMACAAGASLASPNMTSAMLETEPSGRTVTILPGLPTSPESAASACGNSSAGKPDKDSKKEKKGLLKLLSGASTKRKPRVSPPASPTLDVELGAGEAPLQGAVGPELPLGGSHGRVGSCPTDGDGPVAAGTAALAQDAFHRKTSSLDSAVPIAPPPRQACSSLGPVMNEARPVVCERHRVVVSYPPQSEAELELKEGDIVFVHKKREDGWFKGTLQRNGKTGLFPGSFVENI

SEQ ID NO: 10 - DNA, 3149 bp, Drosophila
catttgtatccgcttggccacgagctttggctgcacttggcaaacttaataaattaaacattgaatcctgcctattgcaacgataatataatctgatttagtgcattaagaacgacaagtagcgattataatagtagattttagcatttgagctaaatttatttcccaaccgcgtcttgggattgcgtatgcgtgagccagtacctgcatgtgtgtgtgttttggaatgtggccctgcacgaaattcaaatagtgaccatccttgagattttgcatactggcaagatggacgagcacacgttaaacgacctgttggagtgctccgtgtgtcttgagcgactggacaccacatcgaaggtgctgccatgccagcacaccttctgccgcaaatgcttgcaggacattgtggccagtcagcacaagttgcgatgcccggagtgccgcatcctggtctcttgcaaaattgatgagctgcctccaaacgtcttgctgatgcgaatcttagaaggcatgaaacaaaatgcagcagctggcaaaggagaagaaaagggagaggagactgaaacacagccggaaagggccaaacctcagccgccagcggaatcagtggccccgcctgacaaccaactactccagctgcagtcacatcagcaatctcatcagccggctcgtcacaagcaacgtcgatttctactcccccacgcctatgccctctttgacttcgcctccggtgaagccaccgatctaaagttcaagaaaggggatctgatactgatcaagcatcgcatcgacaacaactggtttgtgggtcaagcgaatggtcaggagggcacatttcccatcaactacgtcaaggtatcggttccgctgcccatgccgcagtgcattgccatgtatgactttaagatggggcccaacgacgaggagggatgcctcgaatttaagaaaagcactgtaatacaggtaatgcgccgagttgatcataattgggcagaaggacgaattggccagaccatcggaatctttccaatagcattcgttgagctgaatgcagcggccaaaaagctgttggacagcgggctacacacccatccattctgccatccaccgaagcaacaggggcagcgggcccttcctccggttccagttattgatcccacggtggtcacggaatccagttcgggatcctccaattccacgccgggcagcagcaattcaagctccacatccagctcgaataactgcagtccgaatcaccaaatctcactgccgaataccccccaacatgtagtagcttccggatcggcgtctgttcgtttccgtgacaagggagcaaaggagaaacgccactcactaaatgctttgctgggaggaggagctccattaagtctgctgcagaccaaccgccattcggctgaaattcttagcctgccccatgaactaagccgcttggaagtttccagctcaacagctctaaaacccacgtcagccccacagacatcgcgtgtacttaagaccactgttcagcagcagatgcaaccgaatttaccctggggatacttagccctgttcccatacaaaccacgccaaacggatgagctggaattaaaaaagggttgtgtttacattgtgaccgaacgatgtgtggacggttggttcaagggaaaaaactggttggacatcactggagtgttcccgggcaactacctgacgcccctgcgcgcccgcgaccagcagcagttaatgcatcaatggaaatatgttccccaaaatgcagacgcccagatggcacaagtacagcagcatccagttgcaccagatgtgcgactcaacaacatgctgtccatgcaaccgcctgatttgccacctcgtcagcagcaggctaccgccacgaccaccagttgctctgtgtggtcgaaaccagtggaggcgctgttcagcagaaaatcggagcccaagcctgaaactgccacagcttcgactacgagcagcagttcctctggagcagtgggacttatgaggagattaactcacatgaaaacacgctccaaatctccgggagcgtccttgcagcaagttccgaaagaagctattagcacaaatgtggaatttacaacaaacccatcagctaaattgcatccagtacatgtaagatccggctcgtgccccagtcagctgcagcacagtcaaccgctcaatgaaactccagcagccaagacagcggcacaacaacagcagttcctacccaagcagctgccttccgcttctacgaacagcgtttcgtacggatcgcaacgcgtgaaaggaagcaaggaacgtcctcacttgatttgcgcgagacaatcattagatgcagctacatttcgcagtatgtacaacaatgccgcgtcgccgccgccacctactacttccgtggccccagctgtctacgccggcggtcagcaacaggtgattcctggaggtggagcgcaatcccagttgcatgccaatatgattattgcacccagccatcggaagtcgcacagcctagatgcgagtcatgtgctgagtcccagcagcaatatgatcacggaggcggccattaaggccagcgccaccactaagtctccttactgcacgagggaaagtcgattccgctgcattgtgccgtatccaccaaacagtgacattgaactagagctacatttgggcgacattatctacgtccagcggaagcagaagaacggctggtataagggcacccatgcccgtacccacaaaaccgggctgttccccgcctcctttgttgaaccggattgttaggaaagttatggttcaaactagaatttattaagcgaaattccaaattacttgtctaaaaggattcaatcgtcggtctattcgggcttccaaatacgcaatctcatatttctcttttcaaaaaagaaaccgttttgtactcttccaatcgaatgggcagctcgccgttgtacttttttatacaatgcttgatcaaaataggctagccatgtaagacttagggaacagttacttaagccttagcgattagttagctagagaaataatctaaccgatccttgtgccctctacaaagttatttgtaatatacgatactcagtaataaaaaaaaaaaaaaaaaaaaaaaaaaa

SEQ ID NO: 11 - protein, 838 aa, Drosophila
MDEHTLNDLLECSVCLERLDTTSKVLPCQHTFCRKCLQDIVASQHKLRCPECRILVSCKIDELPPNVLLMRILEGMKQNAAAGKGEEKGEETETQPERAKPQPPAESVAPPDNQLLQLQSHQQSHQPARHKQRRFLLPHAYALFDFASGEATDLKFKKGDLILIKHRIDNNWFVGQANGQEGTFPINYVKVSVPLPMPQCIAMYDFKMGPNDEEGCLEFKKSTVIQVMRRVDHNWAEGRIGQTIGIFPIAFVELNAAAKKLLDSGLHTHPFCHPPKQQGQRALPPVPVIDPTVVTESSSGSSNSTPGSSNSSSTSSSNNCSPNHQISLPNTPQHVVASGSASVRFRDKGAKEKRHSLNALLGGGAPLSLLQTNRHSAEILSLPHELSRLEVSSSTALKPTSAPQTSRVLKTTVQQQMQPNLPWGYLALFPYKPRQTDELELKKGCVYIVTERCVDGWFKGKNWLDITGVFPGNYLTPLRARDQQQLMHQWKYVPQNADAQMAQVQQHPVAPDVRLNNMLSMQPPDLPPRQQQATATTTSCSVWSKPVEALFSRKSEPKPETATASTTSSSSSGAVGLMRRLTHMKTRSKSPGASLQQVPKEAISTNVEFTTNPSAKLHPVHVRSGSCPSQLQHSQPLNETPAAKTAAQQQQFLPKQLPSASTNSVSYGSQRVKGSKERPHLICARQSLDAATFRSMYNNAASPPPPTTSVAPAVYAGGQQQVIPGGGAQSQLHANMIIAPSHRKSHSLDASHVLSPSSNMITEAAIKASATTKSPYCTRESRFRCIVPYPPNSDIELELHLGDIIYVQRKQKNGWYKGTHARTHKTGLFPASFVEPDC

SEQ ID NO: 12 - DNA, 18 bp, Artificial Sequence
  Sense primer
cttgccttgccagcatac

SEQ ID NO: 13 - DNA, 18 bp, Artificial Sequence
  Anti-sense primer
ctgccagcattccttcag

SEQ ID NO: 14 - DNA, 21 bp, Homo sapiens
aacagaggccttggaaacctg

SEQ ID NO: 15 - DNA, 23 bp, Artificial Sequence
  siRNA sense strand
dtdtcagaggccuuggaaaccug

SEQ ID NO: 16 - DNA, 23 bp, Artificial Sequence
  siRNA anti-sense strand
dtdtcagguuuccaaggccucug

SEQ ID NO: 17 - DNA, 21 bp, Homo sapiens
aaagagcctggagaccttaaa

SEQ ID NO: 18 - DNA, 24 bp, Artificial Sequence
  siRNA sense strand
ddtdtagagccuggagaccuuaaa

SEQ ID NO: 19 - DNA, 24 bp, Artificial Sequence
  siRNA anti-sense strand
ddtdtuuuaaggucuccaggcucu

SEQ ID NO: 20 - DNA, 21 bp, Homo sapiens
aaggattggtatgtgactctg

SEQ ID NO: 21 - DNA, 23 bp, Artificial Sequence
  siRNA sense strand
dtdtggauugguaugugacucug

SEQ ID NO: 22 - DNA, 23 bp, Artificial Sequence
  siRNA anti-sense strand
dtdtcagagucacauaccaaucc

SEQ ID NO: 23 - DNA, 21 bp, Homo sapiens
aagctggattatctcctgttg

SEQ ID NO: 24 - DNA, 24 bp, Artificial Sequence
  siRNA sense strand
ddtdtgcuggauuaucuccuguug

SEQ ID NO: 25 - DNA, 24 bp, Artificial Sequence
  siRNA anti-sense strand
ddtdtcaacaggagauaauccagc

SEQ ID NO: 26 - protein, 41 aa, Homo sapiens
CPVCLERLDASAKVLPCQHTFCKRCLLGIVGSRNELRCPEC

SEQ ID NO: 27 - protein, 56 aa, Homo sapiens
PCAKALYNYEGKEPGDLKFSKGDIIILRRQVDENWYHGEVNGIHGFFPTNFVQIIK

SEQ ID NO: 28 - protein, 60 aa, Homo sapiens
PQCKALYDFEVKDKEADKDCLPFAKDDVLTVIRRVDENWAEGMLADKIGIFPISYVEFNS

SEQ ID NO: 29 - protein, 58 aa, Homo sapiens
SVYVAIYPYTPRKEDELELRKGEMFLVFERCQDGWFKGTSMHTSKIGVFPGNYVAPVT

SEQ ID NO: 30 - protein, 57 aa, Homo sapiens
ERHRVVVSYPPQSEAELELKEGDIVFVHKKREDGWFKGTLQRNGKTGLFPGSFVENI

SEQ ID NO: 31 - DNA, 121 bp, Homo sapiens
tgtccggtgtgtctagagcgccttgatgcttctgcgaaggtcttgccttgccagcatacgttttgcaagcgatgtttgctggggatcgtaggttctcgaaatgaactcagatgtcccgagt

SEQ ID NO: 32 - DNA, 165 bp, Homo sapiens
ccatgtgccaaagcgttatacaactatgaaggaaaagagcctggagaccttaaattcagcaaaggcgacatcatcattttgcgaagacaagtggatgaaaattggtaccatggggaagtcaatggaatccatggctttttccccaccaactttgtgcagattatt

SEQ ID NO: 33 - DNA, 177 bp, Homo sapiens
cctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaac

SEQ ID NO: 34 - DNA, 171 bp, Homo sapiens
agtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtc

SEQ ID NO: 35 - DNA, 169 bp, Homo sapiens
gaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaaca

SEQ ID NO: 36 - DNA, 21 bp, Homo sapiens
aagtccaaaggttccggagac

SEQ ID NO: 37 - DNA, 1502 bp, Homo sapiens
agagacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 38 - DNA, 2217 bp, Homo sapiens
gctgtgtggcccaggcttttctcaaactcctgagggcaagcgatcctcccacctcagcctcctgagtagctgggactacaggcatgtgccactagacctggctctaaagacatatatgacacacgaaaccatttatttttcatttcacaatgtttattcacatatatggtattagtattctaatgtagtgatgcactctaaatttgcattatatttcctagaacatctgaacagagcataggaaattccctattttgccattatcagttctaacaaaaatcttaaaagcactttatcatttcatttccctgcactgtaatttttttaaatgatcaaaaacagtatcataccaaggcttacttatattggaatactattttagaaagttgtgggctgggttgtatttataaatcttgttggtcagatgtctgcaatgagtaaatttagcaccattatcaggaagctttctcaccaatgacaacttcattggaagattttaatgaaagtgtagcatactctagggaaaaaatatgaatattttagcatctatgtattgaaaattatgttgaataaatgtcagactattttttacataacgttgcttctgtttaattttgtcacgttcagaggtggggggtaggagatgtaagcccttgacagcaaaataattccttttgcttgatttcagacagttgcatcagctcctttgttctgtgttcatgttacacttatttaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgacaaaaaaaaaaaaaaaaaa

SEQ ID NO: 39 - DNA, 1684 bp, Homo sapiens
ggccacctcaaggcccacctgagccgcgtctaccccgagcgtccgcgtccagaggaccagaggttaatttattctgggaagctgttgttggatcaccaatgtctcagggacttgcttccaaaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 40 - DNA, 1878 bp, Homo sapiens
ccacgcgtccgggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaa

SEQ ID NO: 41 - DNA, 1864 bp, Homo sapiens
aacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaaaaaaaaaaaaaaaaaa

SEQ ID NO: 42 - DNA, 1871 bp, Homo sapiens
gaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaaaaaaaaaaaaaaaaaaaa

SEQ ID NO: 43 - DNA, 1865 bp, Homo sapiens
agagacgtgaactgtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 44 - DNA, 1884 bp, Homo sapiens
gacgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtctttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtacgatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctgaaaaaaaaaaaaaaaaaaaaaa

SEQ ID NO: 45 - DNA, 1860 bp, Homo sapiens
cgtgaacggtcgttgcagagattgcgggcggctgagacgccgcctgcctggcacctaggagcgcagcggagccccgacaccgccgccgccgccatggagtccgagaccgaacccgagcccgtcacgctcctggtgaagagccccaaccagcgccaccgcgacttggagctgagtggcgaccgcggctggagtgtgggccacctcaaggcccacctgagccgcgtctaccccgagcgtccgcgtccagaggaccagaggttaatttattctgggaagctgttgttggatcaccaatgtctcagggacttgcttccaaagcaggaaaaacggcatgttttgcatctggtgtgcaatgtgaagagtccttcaaaaatgccagaaatcaacgccaaggtggctgaatccacagaggagcctgctggttctaatcggggacagtatcctgaggattcctcaagtgatggtttaaggcaaagggaagttcttcggaacctttcttcccctggatgggaaaacatctcaaggcctgaagctgcccagcaggcattccaaggcctgggtcctggtttctccggttacacaccctatgggtggcttcagctttcctggttccagcagatatatgcacgacagtactacatgcaatatttagcagccactgctgcatcaggggcttttgttccaccaccaagtgcacaagagatacctgtggtctctgcacctgctccagcccctattcacaaccagtttccagctgaaaaccagcctgccaatcagaatgctgctcctcaagtggttgttaatcctggagccaatcaaaatttgcggatgaatgcacaaggtggccctattgtggaagaagatgatgaaataaatcgagattggttggattggacctattcagcagctacattttctgtttttctcagtatcctctacttctactcctccctgagcagattcctcatggtcatgggggccaccgttgttatgtacctgcatcacgttgggtggtttccatttagaccgaggccggttcagaacttcccaaatgatggtcctcctcctgacgttgtaaatcaggaccccaacaataacttacaggaaggcactgatcctgaaactgaagaccccaaccacctccctccagacagggatgtactagatggcgagcagaccagcccctcctttatgagcacagcatggcttgtcttcaagactttctttgcctctcttcttccagaaggccccccagccatcgcaaactgatggtgtttgtgctgtagctgttggaggctttgacaggaatggactggatcacctgactccagctagattgcctctcctggacatggcaatgatgagtttttaaaaaacagtgtggatgatgatatgcttttgtgagcaagcaaaagcagaaacgtgaagccgtgatacaaattggtgaacaaaaaatgcccaaggcttctcatgtgtttattctgaagagctttaatatatactctatgtagtttaataagcactgtacgtagaaggccttaggtgttgcatgtctatgcttgaggaacttttccaaatgtgtgtgtctgcatgtgtgtttgtacatagaagtcatagatgcagaagtggttctgctggtaagatttgattcctgttggaatgtttaaattacactaagtgtactactttatataatcaatgaaattgctagacatgttttagcaggacttttctaggaaagacttatgtataattgctttttaaaatgcagtgctttactttaaactaaggggaactttgcggaggtgaaaacctttgctgggttttctgttcaataaagttttactatgaatgaccctg

SEQ ID NO: 46 - DNA, 1884 bp, Homo sapiens; identical to SEQ ID NO: 44

SEQ ID NO: 47 - DNA, 2140 bp, Homo sapiens
gatacgtggctgccgtctgtccccgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagacaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcagctggctttgattaaaggccatttttaaaacttaaaaactcaacacctcacagattataaaaaaaaaaaaaaaaaaa

SEQ ID NO: 48 - DNA, 3598 bp, Homo sapiens
ggaagtcgatacgtggctgccttctgtccccgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagaaaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcatctggctttgattaaaggccatttttaaaacttaaaaactcaacacctcacagattataatagaaaaagaaatggcctcagtttgatctcgttcagaatgacccagattgtttctgctttgggtgcagctgtttagttcagagttatattacagagaattattttctgagataatcttaaactagaatgttcaaaactaattgataattgaagtatcaagatacgtagaacacctcagagatttttcttcaggaacttccacaaactttgaatccttgtatctttatttggtattcatactactagtagcaaaatacaggttttttgttttgttttgttttggcttcatagagtatctcaaattgaaacttttctgcacaaagaataaaattaaggattttataaactcaaattggcacctactgaattaaaatacataaaatcatttaaatataattcagcatatgggaagtaacattgcactaatatggaaatcactgccagagacagtctattttcttttaatttgttactacttagtcacaaaccccacattattccagtttggaattacttattaaggagaattggaaatacatatgcccatgcttaaattttatagctttaatttgtgttatttctttattgacgggaagaggtacatctttttttccttactgaaaaccaaatatggattaattgcctcaaatttgtataaagtgattggctagtgattcttgttttcaggaagggagagtggtatagatagaaaatgacaaagatggcaatatacacttaatgttgttattgtatgttgttactgaagtacttagatttttaaaatttcaaatcctaaatcacttcttgtaggagggttttcattaactgcagtatatacagttcactacatatgggttgtttgagttttttgtgtgctgtatttctttctgtttttttaatacctggttttgtacatatctaactctgttctcttttggttgttcagaaactggattttttttttttcttaagcagtgcttaatttgtgttttttaattttgattcagaagtagtcccagctcataggtgttcataactgttacatccagaacatttgtcaggctctctgtcagcttttcatgtacatatggtatagaaaccatggagttaggcacttccctggaatttttttttttatgagaaaaatactgtatttaaaatgtaaaataaacttttaaaaagcaggcactaatatatatttcttccagcctttgattacaaatttgtccttgcacatgttaagatgaattatctcctaaaaatatcattgttcttgggagcagtgtatgttactttacatagcagcggttcctgtcatgtgttcatgttcagaaatatttttggttttaaactttcttattgcctttggctgttgattagtacagtacaagttgcgatttcaaaaagatcttgaaagtaatatatttaatcaattaaaatgtttatctgtaaaaaaaaaaaaaaaaaa

SEQ ID NO: 49 - DNA, 3598 bp, Homo sapiens; identical to SEQ ID NO: 48

SEQ ID NO: 50 - DNA, 3049 bp, Homo sapiens
cggacgcgtgggtgccatctcttttcaacatatgttgcgtcccacaaaatagagaaggaagagcaagaaaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcagctggctttgattaaaggccatttttaaaacttaaaaactcaacacctcacagattataatagaaaaagaaatggcctcagtttgatctcgttcagaatgacccagattgtttctgctttgggtgcagctgtttagttcagagttatattacagagaattattttctgagataatcttaaactagaatgttcaaaactaattgataattgaagtatcaagatacgtagaacacctcagagatttttcttcaggaacttccacaaactttgaatccttgtatctttatttggtattcatactactagtagcaaaatacaggttttttgttttgttttgttttgttttggcttcatagagtatctcaaattgaaacttttctgcacaaagaataaaattaaggattttataaactcaaattggcacctactgaattaaaatacataaaatcatttaaatataattcagcatatgggaagtaacattgcactaatatggaaatcactgccagagacagtctattttcttttaatttgttactacttagtcacaaaccccacattattccagtttggaattacttattaaggagaattggaaatacatatgcccatgcttaaattttatagctttaatttgtgttatttctttattgacgggaagaggtacatctttttttccttactgaaaacaaatatggattaattgcctcaaatttgtataagtgattggctagtgattcttgttttcagaagggagagtggtatagatagaaaatgacaaagatggcaatatacacttaatgttgttattgtatgttgttactgaagtacttagatttttaaaatttcaaatcctaaatcacttcttgtaggagggttttcattaactgcagtatatacagttcactacatatgggttgtttgagttttttgtgtgctgtatttctttctgttttttaatacctggttttgtacatatctaactctgttctcttttggttgttcagaaactggatttttttttttcttaagcagtgcttaatttgtgttttttaattttgattcagaagtagtcccagctcataggtgttcatactgttacatccagaacatttgtcaggctctctgtcagctttcatgtacatatggtatagaaaccatggagttaggcacttcctggattttttttttatgagaaaaatactgtatttaaaatgtaaaataaacttttaaaaagcaggcactaatatatatttcttccagcctttgattacaaatttgtccttgcacatgttaagatgaattatctcctaaaaatatcattgttcttgggagcagtgtatgttactttacatagcagcggttcctgtcatgtgttcatgtcagagtatttttggttttaaactttcttattgcctttggctgttgattagtacagtacaagtgcgatttcaaaaagatcttgaaagtaatatatttaatcaattaaaatgtttatctgtaaaaaaaaaaaaaaaaaaaaaaaa

SEQ ID NO: 51 - DNA, 3572 bp, Homo sapiens
  misc_feature 3240: n = A,T,C or G
ccgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcctcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagacaaaaaaaggaaggaggaagaggagcgaaggcggcgtgaagaggaagaaagagagcgtctgcaaaaggaggaagagaaacgtaggagagaagaagaggaaaggcttcgacgggaggaagaggaaaggatacggatagaagaagaaaggcttcggttggagcagcaaaagcagcagataatggcagctttaaactcccagactgccgtgcagttccagcagtatgcagcccaacggtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgccagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagactttcaaagagaagattcagcaggatgcagattccgtgattacagtggccgaggagaagtggtcactgttcgagtacccacccatgaagaaggatcatatctcttttgggaatttgccacagacaattgtgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactaggggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcagctggctttgattaaaggccatttttaaaacttaaaaactcaacacctcacagattataatagaaaaagaaatggcctcagcttgatctcgttcagaatgacccagattgtttctgccttgggtgcagctgtttagttcagagttatattacagagaattattttctgagataatcttaaactagaatgttcaaaactaattgataattgaagtatcaagatacgtagaacacctcagagatttttcttcaggaacttccacaaactttgaatccttgtatctttatttggtattcatactactagtagcaaaatacaggttttttgttttgttttgttttgttttggcttcatagagtatctcaaattgaaacttttctgcacaaagaataaaattaaggattttataaactcgaattggcacctactgaattaaaatacataaaatcatttaaatataattcagcatatgggaagtaacattgcactaatatggaaatcactgccagagacagtctattttcttttaatttgttactacttagtcacaaccccacattattccagtttggaattacttattaaggagaattggaaatacatgtgcccatgcttaaattttatagctttaatttgtgttatttctttattgacgggaagaggtacatctttttttccttactgaaaacaaatatggattaattgcctcaaatttgtataaagtgattggctagtgattcttgttttcagaggggagagtggtatagatagaaaatgacgaagatggcaatatacacttaatgttgttattgtatgttgttactgaagacttagatttttaaaatttcaaatcctaaatcacttcttgtaggggggttttcattaactgcagtatatacagttcactacatatgggttgtttgagttttttgtgtgctgtatttctttctgttttttaatacctggttttgtacatatctaactctgttctcttttggttgttcagaaactggatttttttcttcttaagcagtgcttaatttgtgttttttaattttgattcagaagtagtcccagctcataggcgttcatactgttacatccagaacatttgtcaggctctctgtcagctttcatgtacatatggtatagaaaccatggagttaggcacttcctggattttttttatgagaaaaatnctgtatttaaaatgtaaaataaacttttaaaaagcaggcactaatatatatttcttccagcctttgattacaaatttgtccttgcacatgttaagatgaattatctcctaaaaatatcattgttcttgggagcagtgtatgttactttacatagcagcggttcctgtcatgtgttcatgtcacgaatatttttggttttaaactttcttattgcctttggctgttgattagtacagtacaagtgcgatttcaaaaagatcttgaaagtaatatatttaatcaattaaaatgtttatctggaaaaaaaaaaaaaaaaaaaaaaaaaaaaaaaa

SEQ ID NO: 52 - DNA, 3560 bp, Homo sapiens
tccgtccccgctgaggaggtgcagcagcgggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagaaaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccggaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttacccggctgatggttagccccttgctgcctgctccatgtgtcttatgagagcccgtagttacagtgtcctctaatttgaaatccataagttaacaagtctatatcaggtgcagctggctttgattaaaggccatttttaaaacttaaaaactcaacacctcacagattataatagaaaaagaaatggcctcagtttgatctcgttcagaatgacccagattgtttctgctttgggtgcagctgtttagttcagagttatattacagagaattattttctgagataatcttaaactagaatgttcaaaactaattgataattgaagtatcaagatacgtagaacacctcagagatttttcttcaggaacttccacaaactttgaatccttgtatctttatttggtattcatactactagtagcaaaatacaggttttttgttttgttttgttttggcttcatagagtatctcaaattgaaacttttctgcacaaagaataaaattaaggattttataaactcaaattggcacctactgaattaaaatacataaaatcatttaaatataattcagcatatgggaagtaacattgcactaatatggaaatcactgccagagacagtctattttcttttaatttgttactacttagtcacaaaccccacattattccagtttggaattacttattaaggagaattggaaatacatatgcccatgcttaaattttatagctttaatttgtgttatttctttattgacgggaagaggtacatctttttttccttactgaaaacaaatatggattaattgcctcaaatttgtataagtgattggctagtgattcttgttttcagaagggagagtggtatagatagaaaatgacaaagatggcaatatacacttaatgttgttattgtatgttgttactgaagtacttagatttttaaaatttcaaatcctaaatcacttcttgtaggagggttttcattaactgcagtatatacagttcactacatatgggttgtttgagttttttgtgtgctgtatttctttctgttttttaatacctggttttgtacatatctaactctgttctcttttggttgttcagaaactggatttttttttcttaagcagtgcttaatttgtgttttttaattttgattcagaagtagtcccagctcataggtgttcatactgttacatccagaacatttgtcaggctctctgtcagctttcatgtacatatggtatagaaaccatggagttaggcacttcctggatttttttttttatgagaaaaatactgtatttaaaatgtaaaataaacttttaaaaagcaggcactaatatatatttcttccagcctttgattacaaatttgtccttgcacatgttaagatgaattatctcctaaaaatatcattgttcttgggagcagtgtatgttactttacatagcagcggttcctgtcatgtgttcatgtcagaatatttttggttttaaactttcttattgcctttggctgttgattagtacagtacaagtgcgatttcaaaaagatcttgaaagtaatatatttaatcaattaaaatgtttatctgtcaaaaaaaaaaaaaaa

SEQ ID NO: 53 - DNA, 1995 bp, Homo sapiens
cgctgaggaggtgcagcagccggagatggcggcggtgctgaacgcagagcgactcgaggtgtccgtcgacggcctcacgctcagcccggacccggaggagcggcctggggcggagggcgccccgctgctgccgccaccgctgccaccgccctcgccacctggatccggtcgcggcccgggcgcctcaggggagcagcccgagcccggggaggcggcggctgggggcgcggcggaggaggcgcggcggctggagcagcgctggggtttcggcctggaggagttgtacggcctggcactgcgcttcttcaaagaaaaagatggcaaagcatttcatccaacttatgaagaaaaattgaagcttgtggcactgcataagcaagttcttatgggcccatataatccagacacttgtcctgaggttggattctttgatgtgttggggaatgacaggaggagagaatgggcagccctgggaaacatgtctaaagaggatgccatggtggagtttgtcaagctcttaaataggtgttgccatctcttttcaacatatgttgcgtcccacaaaatagagaaggaagagcaagaaaaaaaaaaggaaggaggaagaggagcgaaggcggcgtgaagaggaagaaagagaacgtctgcaaaaggaggaagagaaacgtaggagagaagaagaggaaaggcttcgacgggaggaagaggaaaggagacggatagaagaagaaaggcttcggttggagcagcaaaagcagcagataatggcagctttaaactcccagactgccgtgcagttccagcagtatgcagcccaacagtatccagggaactacgaacagcagcaaattctcatccgccagttgcaggagcaacactatcagcagtacatgcagcagttgtatcaagtccagcttgcacagcaacaggcagcattacagaaacaacaggaagtagtagtggctgggtcttccttgcctacatcatcaaaagtgaatgcaactgtaccaagtaatatgatgtcagttaatggacaggccaaaacacacactgacagctccgaaaaagaactggaaccagaagctgcagaagaagccctggagaatggaccaaaagaatctcttccagtaatagcagctccatccatgtggacacgacctcagatcaaagacttcaaagagaagattcagcaggatgcagattccgtgattacagtgggccgaggagaagtggtcactgttcgagtacccacccatgaagaaggatcatatctcttttgggaatttgccacagacaattatgacattgggtttggggtgtattttgaatggacagactctccaaacactgctgtcagcgtgcatgtcagtgagtccagcgatgacgacgaggaggaagaagaaaacatcggttgtgaagagaaagccaaaaagaatgccaacaagcctttgctggatgagattgtgcctgtgtaccgacgggactgtcatgaggaggtgtatgctggcagccatcaatatccagggagaggagtctatctcctcaagtttgacaactcctactctttgtggcggtcaaaatcagtctactacagagtctattatactagataaaaatgttgttacaaagtctggagtctagggttgggcagaagatgacatttaatttggaaatttctttttacttttgtggagcattagagtcacagtttaccttattgatattggtctgatggtttgtgaactcttgctgggaatcaaaatttccttgagactctttagcattcatactttggggttaaaggagattcctcagactcatccagcccttgggtgctgaccagcagagtcactagtggatgctgaagttacatgagctacatgttaaatatttaaagtctccaaaataaaacaccccaacgttgaccttaaaaaaaaaaaaaaaaaaaaaaaaaaaaaaaaaaaaaaaaaaaaaaaaaaaaaaaaaaaaaaaaaa

SEQ ID NO: 54 - DNA, 1319 bp, Homo sapiens
gaattccgttgctgtcggagcccgtagttacagtgtcctctaatttgaaatccataagttaccaagtctatatcaggtacagctggctttcattaaaggccatttttaaaacttcaaaaactcaacacctcacagattataatagaaaaagaaatggcctcagtttgatctcgttcagaatgacccagattgttttctgctttgggtgcagctgtttagttcagagttatattacagagaattattttctgagaaatcttaaaactagaatgttcaaaactaattcgataattgaagtatcaagatacgtagaacacctcagagatttttcttcaggaacttccacaaactttagaatccttgtatctttatttggtattcatactactagtcgcaaaatacaggttttttgttttgttttgttttgttttggcttcatagagtatctcaaattgaaacttttctgcccaaagaataaaattaaggattttataaaactcaaattggcacctactgaattaaaatacataaaatgcattaaatataattcagcatatggcagtaacattgcactaatatggaaatcactgccagagacagtctattttcttttaatttgttactacttagtcacaaccccacattattccagtttggaattacttattaaggagaattggaaatacatatgcccatgcttaaattttatagctttaatttgtgttatttctttattgacgggaagaggtacatctttttttccttactcaaaacaaatatggattaattgcctcaaatttgtataagtgattggctagtgattcttgttttcagagggagagtggtatagatagaaaatgacaaagatggcaatatacacttaatgttgttattgtatgttgttactgaagtacttagatttttaaaatttcaaatcctaaatcacttcttgtaggagggttttcattaactgcagatatacagttcactacatatgggttgtttgagttttttgtgtgctgtatttctttctgttttttaatacctggttttgtacatatctaactctgttctcttttggttgttcagaaactggattttttttttcttaagcagtgcttaatttgtgttttttaattttgattcagaagtagtcccagctcataggtgttcatactgttacatccagaacatttgtcaggctctctgtcagctttcatgtacatatggtatagaaaccatggagttaggcacttcctggattttttttttatgagaaaaatactgtatttaaaatgtaaaataaacttttaaaaagc

SEQ ID NO: 55 - DNA, 3354 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggtacgtatagaatataatttcctttgtgatgtacatcttaatggtcagaatttaaaggcaaaatttcatgccattgtactgaaaatacattaaggttttgtgttatcctctaggagatgtttttgattcagcctctgatcccgtgccattaccacctgccaggcctccaactcgggacaatccaaagcatggttcttcactcaacaggacgccctctgattatgatcttctcatccctccattaggttgaaacctttaaaaaagttttgaacaacccacccctccttcttttaatttcagaattttcagaattcagagttcagtataacacagactcactgggttgtgaatttgcctgaaatttgaatgggttctccaggtgccggtgactcccaagttcacgagaccattactccatgtagatgattaaggtagtagtgtagtagttgggcatcagtcaggttttaagcaagttgttttgtccatactaaatgtagtctaaaaacacatgagagctttgtgctctagtagttttgaagtgatgacttgaagtgttgagattttctttaagtataataattcttaataaatatgaacttgcttttcttgcagcatgagcaccagttccacttacgctaattaaattatgcaaaattaaatagttgtatgtagagaactgataataaattctgttttattctaatcattacaactgtaacacattcaaaaaaaaaaa

SEQ ID NO: 56 - DNA, 3928 bp, Homo sapiens
agcggagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctggcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggatccctttgatccaagagatgaaggctccaggtgttgcagcatcattgacccctttggcatgccgatgctcgacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacgggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccaaagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacaatttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaagtatgtattcttcactactgagtttcttctttggaaaccatcactattgagagatgggaaaaacctgaatgtataaagcatttatttgtcaataaactgccttttgtaaggggttttcacaaaaaaaaaaaaaaaa

SEQ ID NO: 57 - DNA, 3982 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacaatttggcaaagttgcagtttgttcttgtttttagtttagttttgttttggtgttttgatacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaagtatgtattcttcactactgagtttcttctttggaaaccatcactattgagagatgggaaaaacctgaatgtataaagcatttatttgtcaataaactgccttttgtaaggggttttcacataaaaaaaaaaaaa

SEQ ID NO: 58 - DNA, 3241 bp, Homo sapiens
ctgggtcctgtgtgtgccacaggggtggggtgtccagcgagcggtctcctcctcctgctagtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggttgaaacctttaaaaaagttttgaacaacccacccctccttcttttaatttcagaattttcagaattcagagttcagtataacacagactcactgggttgtgaatttgcctgaaatttgaatgggttctccaggtgccggtgactcccaagttcacgagaccattactccatgtagatgattaaggtagtagtgtagtagttgggcatcagtcaggttttaagcaagttgttttgtccatactaaatgtagtctaaaaacacatgagagctttgtgctctagtagttttgaagtgatgacttgaagtgttgagattttctttaagtataataattcttaataaatatgaacttgcttttcttgcagcatgagcaccagttccacttacgctaattaaattatgcaaaattaaatagttgtatgtagagaactgataataaattctgttttattctaatcattacaactgtaacacattcaaaaaaaaaaa

SEQ ID NO: 59 - DNA, 3354 bp, Homo sapiens; identical to SEQ ID NO: 55

SEQ ID NO: 60 - DNA, 2088 bp, Homo sapiens
agtgctgctgcggcgtcccgcggcctccccgagtcgggcgggaggggagagcgggtgtggatttgtcttgacggtaattgttgcgtttccacgtctcggaggcctgcgcgctgggttgctccttcttcgggagcgagctgttctcagcgatcccactcccagccggggctccccacacacactgggctgcgtgcgtgtggagtgggacccgcgcacacgcgtgtctctggacagctacggcgccgaaagaactaaaattccagatggcaaactcaatgaatggcagaaaccctggtggtcgaggaggaaatccccgaaaaggtcgaattttgggtattattgatgctattcaggatgcagttggaccccctaagcaagctgccgcagatcgcaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcatcagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaaggagaccgatgcttgctcaggatgtcgacagctgtggcttccttgtttttgctagccatatttttaaatcagggttgaactgacaaaaataatttaaagacgtttacttcccttgaactttgaacctgtgaaatgctttaccttgtttacagtttggcaaagttgcagtttgttcttgtttttagtttagttttgttttggtgttttgtacctgtactgtgttcttcacagaccctttgtagcgtggtcaggtctgctgtaacatttcccaccaactctcttgctgtccacatcaacagctaaatcatttattcatatggatctctaccatccccatgccttgcccaggtccagttccatttctctcattcacaagatgctttgaaggttctgattttcaactgatcaaactaatgcaaaaaaaaaaaaaaaaaaaaaaaaaag

SEQ ID NO: 61 - DNA, 1446 bp, Homo sapiens
  misc_feature 4, 10, 12, 13, 58, 74, 136, 206, 222, 237, 254, 385,1336, 1344, 1347, 1350, 1380, 1392, 1395, 1400, 1445: n = A,T,C or G
cgtntttggnanncactacaggggatgtttaatacacactcacaatgcgcatgatgtntataactatctattcnatgatgtaagataccccactcaaacccataaaaaagagcatctttaatacgactcactatanggcgagcgcacgccatggcaggtacccatacgacgtaccagattacgctcatatggccatggaggccagngaattccacccaagcngtggtatcaacgcanagtggactctgacccantgcttatgcggaaacacagacgccatgatttgcctttagaaggagctaaggtctcttccaatggtcaccttggaagtgaagaatatgatgttcctccccggctttctcctcctcctccagttaccaccctnctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacaggacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaagaaaaccgatgcttgctcaggatgtcnacagctgnggnctnccttgtttttgctagccatttttttaaatnagggttgaactnganaaaantatttaaaaacgtttacctcccttgaactttgaacctgggaaagnc

SEQ ID NO: 62 - DNA, 1203 bp, Homo sapiens
actctgacccagtgcttatgcggaaacacagacgccatgatttgcctttagaaggagctaaggtctcttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcttgtgataatggtcactgtatgctgaatggaacacatggtccatcttcagagaagaaatcaaacatccctgacttaagcatatatttaaagggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcccgctagaaggttaccaggtgaaaatgtcaaaactaacaggacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatagcagccagaactgtagacaccaaaatggaaagcaatcgatgtattccaagagtgtggaaataaagagaactgagatggaattcaagagagaagtgtctcctcctcgtgtagcagcttgagaagaggcttgggagtgcagcttctcaaagaaaaccgatgcttgctcaggatgtcgacagctgtggcttccttgtttttgctagccatttttttaaatcagggttgaactggaaaaaattatttaaaaacgtttacctcccttgaactttgaacctgggaaaggc

SEQ ID NO: 63 - DNA, 639 bp, Homo sapiens
atggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtgtcgaacgttcaccactaacggctctgacaccaccataccaatggacgaatcactgcagtttaactcctccctccagaaagttcacaacggcatggatgacctcattttagatgggcacaatattttagatggactgaggacccagagactgaccttgaaggggactcagaagaagatccctgacattgccaacatgctgggcttgtccaacacagtgatgcggctcatcgagaagcgggctttccaggacaagtactttatgataggtgggatgctgctgacctgtgtggtcatgttcctcgtggtgcagtacctgacatga

SEQ ID NO: 64 - DNA, 2209 bp, Homo sapiens
ggcctgccgggccggcgacatggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtctcgaaccttcaccactaacgactctgacaccaccataccaatggacgaatcactgcagtttaactcctccctccagaaagttcacaacggcatggatgacctcattttagatgggcacaatattttagatggactgaggacccagagactgaccttgaaggtggggtccctgctgggggacagagagaaggcctcttgttttagcctcatccaacagtttagtaactgtgtttatattttgattacgtgtcctcaaattgtgatattttgatgacaagacagagcccttgagtttgggatcctttctgttggagttgagttattgtgagcctgaaagtacccagttcctttgccagtgcttgaaacaaaccatgaagtggcctctcttaggatccaggtcttttcccatttactgaacttatcatgaaagtgagtgctactacgaggggtccaatcacaggctgagaaattgtgttacagaatctactcttggaagaatgaagacgtggctgtcctttggtacctcgctttaaggtggctttcccttaggacccctactgtggactgccttataactaaaaccttttgtattttagtaactgaatccccactgtgcagtgttagggctgcctggttgtttgcagtagattagagctttagaagcttctagagcttctaaagcccgtgctggtgatcccagcgactcttcactccctagccttaggtattcctagaagccctgaccagttggcactgctgagactccagcccctgggagtggtttacagaaacattacacagactctgatgtcagtcatgatgtttcagcctctgcccttttcctgtatcaaccctgatggataatagggcgtgggttctgtctgttatcagggtgtggtcccctgtgaatgaagcactcccagccactgagctgtgagaaacagtcactcggaagtgtgagctttatcttagtttttgttggatcatgttgagtctgtcagctccacaggacttcagtacgtttctgaacagtccctgccatctctacgggggagagggtcaggcaagctgcaagtgacactcacctcctgctgacagttgcagtgtctcagatggcctggaagggtggtctccagcagcctgctgggcgctcccctttcatgagagccacctgcagtgacctgaactgatacatgttgattagtctgccctttctttagaaaactgctactctccttttcatatctcagaaaaacagtagaggccttttaggaccaaactccatgtcacactgatgaagagccagtgggggttagagcgtcctgttaaggcacatgctagcttcccactcaagtctggcagcgctggggcatcagcacacctcttgccacccacactgataccagaggggaaggctgtgaggtggctgggggttgagacttgaggtttctaactttcctctgcacacctgtggctacctggtgtttgtctcttgattccctccacctgcctcacaccctgcctccgtcgggattttccacctacaccattcaaaaggaacataggagagggcatgaaggggctaggctgaagcactctgatgactggggccaatttgtggctgaaaatgaatacattttttgaaatttatggtcattttcaagtgatttagaaggttgatccttagcctcatacagtgatgaaataatctgtgtgttcagagccaagcaggactttagcaagagtctgattgtattgtcactatctcggggaaaaaaaaatacaaatacatttctctgatctctgatggcaatgaagtttgacttgtaaaaaaaaaaaaaaaaaaaaaaaaaaaaaaaaaaaaaaaaaaaaa

SEQ ID NO: 65 - DNA, 886 bp, Homo sapiens
cccacgcgtccggttccgaggaagccagagccggagccgtggcctgccgggccggcgacatggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtctcgaaccttcaccactaacgactctgacaccaccataccaatggacgaatcactgcagtttaactcctccctccagaaagttcacaacggcatggatgacctcattttagatgggcacaatattttagatggactgaggacccagagactgaccttgaaggggactcagaagaagatccttgacattgccaacatgctgggcttgtccaacacagtgatgcggctcatcgagaagcgggctttccaggacaagtactttatgataggtgggatgctgctgacctgtgtggtcatgttcctcgtggtgcagtacctgacatgagccagccacgctcagtggctgaacagcattcccacagcctgcaagtgtgtgtgtgtgtgaaagagagaggggggcccagaggccgccttttgaaatgtttgcctgtctgaactgtgaagacacttgggagtgattgtggtctaatttccaaaaaaaaaaaaaaaaaaaaaaaaaaaaaaaaaaaaaaa

SEQ ID NO: 66 - DNA, 890 bp, Homo sapiens
gttccgaggaagccagagccggagccgtggcctgccgggccggcgacatggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtctcgaaccttcaccactaacgactctgacaccaccataccaatggacgaatcactgcagtttaactcctccctccagaaagttcacaacggcatggatgacctcattttagatgggcacaatattttagatggactgaggacccagagactgaccttgaaggggactcagaagaagatccttgacattgccaacatgctgggcttgtccaacacagtgatgcggctcatcgagaagcgggctttccaggacaagtactttatgataggcacccaaggatcctgccagacagcacactttggaggaaggtctgcagggagcagctgagccatttgttcttgaactctgggaggcagaagtccccgcacccatcatgcgtggactgataggacatcttttcgtggtgtgcaccagtgctttccacacttgacagtggttggctttgatgaaccctcatgctgcaccttcagagccagtcctctagtttggaataaaaattgcagaggtggaaaaaaaaaaaaaaaaaaa

SEQ ID NO: 67 - DNA, 874 bp, Homo sapiens
agccggagccgtggcctgcggggccggcgacatggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtctcgaaccttcaccactaacgactctgacaccaccataccaatggacgaatcactgcagtttaactcctccctccagaaagttcacaacggcatggatgacctcattttagatgggcacaatattttagatggactgaggacccagagactgaccttgaaggggactcagaagaagatccttgacattgccaacatgctgggcttgtccaacacagtgatgcggctcatcgagaagcgggctttccaggacaagtactttatgataggcacccaaggatcctgccagacagcacactttggaggaaggtctgcagggagcagctgagccatttgttcttgaactctgggaggcagaagtccccgcacccatcatgcgtggactgataggacatcttttcgtggtgtgcaccagtgctttccacacttgacagtggttggctttgatgaaccctcatgctgcaccttcagagccagtcctctagtttggaataaaaattgcagaggtggaaaaaaaaaaaaaaaaaaa

SEQ ID NO: 68 - DNA, 874 bp, Homo sapiens
gttccgaggaagccagagccggagccgtggcctgccgggccggcgacatggatcccctgttccagcaaacgcacaagcaggtccacgagatccagtcttgcatgggacgcctggagacggcagacaagcagtctgtgcacatagtagaaaacgaaatccaagcaagcatagaccagatattcagccgtctagaacgtctggagattttgtccagcaaggagccccctaacaaaaggcaaaatgccagacttcgggttgaccagttaaagtatgatgtccagcacctgcagactgcgctcagaaacttccagcatcggcgccatgcaagggagcagcaggagagacagcgagaagagcttctgtctcgaaccttcaccactaacgactctgacaccaccataccaatggacgaatcactgcagtttaactcctccctccagaaagttcacaacggcatggatgacctcattttagatgggcacaatattttagatggactgaggacccagagactgaccttgaaggggactcagaagaagatccttgacattgccaacatgctgggcttgtccaacacagtgatgcggctcatcgagaagcgggctttccaggacaagtactttatgataggtgggatgctgctgacctgtgtggtcatgttcctcgtggtgcagtacctgacatgagccagccacgctcagtggctgaacagcattcccacagcctgcaagtgtgtgtgtgtgtgaaagagagaggggggcccagaggccgccttttgaaatgtttgcctgtctgaactgtgaagacacttgggagtgattgtggtctaatttccaaaaaaaaaaaaaaaaaaaaaaaaaaaaaaaaaaaaaaa

SEQ ID NO: 69 - DNA, 621 bp, Homo sapiens
atggctgcaaataagcccaagggtcagaattctttggctttacacaaagtcatcatggtgggcagtggtggcgtgggcaagtcagctctgactctacagttcatgtacgatgagtttgtggaggactatgagcctaccaaagcagacagctatcggaagaaggtagtgctagatggggaggaagtccagatcgatatcttagatacagctgggcaggaggactacgctgcaattagagacaactacttccgaagtggggaggggttcctctgtgttttctctattacagaaatggaatcctttgcagctacagctgacttcagggagcagattttaagagtaaaagaagatgagaatgttccatttctactggttggtaacaaatcagatttagaagataaaagacaggtttctgtagaagaggcaaaaaacagagctgagcagtggaatgttaactacgtggaaacatctgctaaaacacgagctaatgttgacaaggtattttttgatttaatgagagaaattcgagcgagaaagatggaagacagcaaagaaaagaatggaaaaaagaagaggaaaagtttagccaagagaatcagagaaagatgctgcattttataa

SEQ ID NO: 70 - DNA, 2774 bp, Homo sapiens
ccgctccccagagcaaagcgtcggagtcctcctcctccttctcctcctcctcctcctcctcctccagccgcccaggctcccccgccacccgtcagactcctccttcgaccgctcccggcgcggggccttccaggcgacaaggaccgagtaccctccggccggagccacgcagccgcggcttccggagccctcggggcggcggactggctcgcggtgcagattcttcttaatcctttggtgaaaactgagacacaaaatggctgcaaataagcccaagggtcagaattctttggctttacacaaagtcatcatggtgggcagtggtggcgtgggcaagtcagctctgactctacagttcatgtacgatgagtttgtggaggactatgagcctaccaaagcagacagctatcggaagaaggtagtgctagatggggaggaagtccagatcgatatcttagatacagctgggcaggaggactacgctgcaattagagacaactacttccgaagtggggaggggttcctctgtgttttctctattacagaaatggaatcctttgcagctacagctgacttcagggagcagattttaagagtaaaagaagatgagaatgttccatttctactggttggtaacaaatcagatttagaagataaaagacaggtttctgtagaagaggcaaaaaacagagctgagcagtggaatgttaactacgtggaaacatctgctaaaacacgagctaatgttgacaaggtattttttgatttaatgagagaaattcgagcgagaaagatggaagacagcaaagaaaagaatggaaaaaagaagaggaaaagtttagccaagagaatcagagaaagatgctgcattttataatcaaagcccaaactcctttcttatcttgaccatactaataaatataatttataagcattgccattgaaggcttaattgactgaaattactttaacattttggaaattgttgtatatcactaaaagcatgaattggaactgcaatgaaagtcaaatttactttaaaaagaaattaatatggcttcaccaagaagcaaagttcaacttatttcataattgcctacatttatcatggtcctgaatgtagcgtgtaagcttgtgtttcttgggcagtctttcttgaaattgaagaggtgaaatgggggtggggagtgggaggaaaggtgacttcctctggtgtttattataaagcttaaattttatatcattttaaaatgtcttggtcttctactgccttgaaaaatgacaattgtgaacatgatagttaaactaccactttttttaaccattattatgcaaaatttagaagaaaagttattggcatggttgttgcatatagttaaactgagagtaattcatctgtgaatctgctttaattacctggtgagtaacttagaaaagtggtgtaaacttgtacatggaattttttgaatatgccttaatttagaaactgaaaaatatctggttatatcattctgggtgtgttcttactgacaccaggggtccgctgccccatgtgtcctggtgagaaaatatatgcctggcacagcttttgtatagaaaattcttgagaagtaactgtccgctagaagtctgtccaaatttaaaatgtgtgccatattctggttcttgaaaataagattccagagctctttgatcgcttttaataaactgcaagttcattttaaatgaagggccagcatatatacttgcaagataattttcagctgcaaggattcagcaccagttatgtttgaatgaaccctccttttctctgagattctggtccctggaaatccctttctgctagtggtgagcatgtaagtgttaagtttttaatctgggagcagggcataggaagaaaatgtcagtagtgctaatgcattttgcactagaacgcttcgggaaaatattcatgcttgccatctgttcatttctaaatttatattcataaagttacagtttgatacaggaattattaggagtaattcttttctgtttctgtttataatgaagaacactgtagctacattttcagaagttaacatcaagccatcaaacctgggtatagtgcagaaaacgtggcacacactgaccacacattaggctgtgtcaccattgtgtggtgtacctgctggaagaattctagcatgctacttggggacataatttcagtgggaaatatgccactgaccgattttttttttttcctctttgcagtggggctaggacagttgattcaacaaagtatttttttcttttttctcagtcctaatttgaacaggtcaaagatgtgttcaggcattccaggtaacaggtgtgtatgtaaagttaaaaataggctttttaggaactcactctttagatatttacatccagcttctcatgttaaatatttgtccttaaagggtttgagatgtacatctttcatttcgtatttctcataggctatgccatgtgcggaattcaagttaccaatgtaacactggccagcgggcccagcaatctccatgtgtacttattacagtcttatttaaccaggggtcctaaccactaacattgtgactttgctttgagacctttcctctcctgggtactgaggtgctatgaagccaactgacaaagatgcatcacgtgtcttaggctgatgccactacccgatttgtttatttgcaatttgagccatttaaagaccaataaacttccttttttaaaaaaaaaaaaaaaaaaaaaaaaaa

SEQ ID NO: 71 - DNA, 470 bp, Homo sapiens
ataatcaaagcccaaactcctttcttatcttgaccatactaataaatataatttataagcattgccattgaaggcttaattgactgaaattactttaacattttggaaattgttgtatatcactaaaagcatgaattggaactgcaatgaaagtcaaatttactttaaaaagaaattaatatggcttcaccaagaagcaaagttcaacttatttcataattgcctacatttatcatggtcctgaatgtagcgtgtaagcttgtgtttcttgggcagtctttcttgaaattgaagaggtgaaatgggggtggggagtgggaggaaaggtgacttcctctggtgtttattataaagcttaaattttatatcattttaaaatgtcttggtcttctactgccttgaaaaatgacaattgtgaacatgatagttaaactaccactttttttaaccattattatgcaaaaaaaaaa

SEQ ID NO: 72 - DNA, 630 bp, Homo sapiens
atggtcgactacctagcaaataagcccaagggtcagaattctttggctttacacaaagtcatcatggtgggcagtggtggcgtgggcaagtcagctctgactctacagttcatgtacgatgagtttgtggaggactatgagcctaccaaagcagacagctatcggaagaaggtagtgctagatggggaggaagtccagatcgatatcttagatacagctgggcaggaggactacgctgcaattagagacaactacttccgaagtggggaggggttcctctgtgttttctctattacagaaatggaatcctttgcagctacagctgacttcagggagcagattttaagagtaaaagaagatgagaatgttccatttctactggttggtaacaaatcagatttagaagataaaagacaggtttctgtagaagaggcaaaaaacagagctgagcagtggaatgttaactacgtggaaacatctgctaaaacacgagctaatgttgacaaggtattttttgatttaatgagagaaattcgagcgagaaagatggaagacagcaaagaaaagaatggaaaaaagaagaggaaaagtttagccaagagaatcagagaaagatgctgcattttataa

SEQ ID NO: 73 - DNA, 1965 bp, Homo sapiens
agaatggaaaaaagaagaggaaaagtttagccaagagaatcagagaaagatgctgcattttataatcaaagcccaaactcctttcttatcttgaccatactaataaatataatttataagcattgccattgaaggcttaattgactgaaattactttaacattttggaaattgttgtatatcactaaaagcatgaattggaactgcaatgaaagtcaaatttactttaaaaagaaattaatatggcttcaccaagaagcaaagttcaacttatttcataattgcctacatttatcatggtcctgaatgtagcgtgtaagcttgtgtttcttgggcagtctttcttgaaattgaagaggtgaaatgggggtggggagtgggaggaaaggtgacttcctctggtgtttattataaagcttaaattttatatcattttaaaatgtcttggtcttctactgccttgaaaaatgacaattgtgaacatgatagttaaactaccactttttttaaccattattatgcaaaatttagaagaaaagttattggcatggttgttgcatatagttaaactgagagtaattcatctgtgaatctgctttaattacctggtgagtaacttagaaaagtggtgtaaacttgtacatggaattttttgaatatgccttaatttagaaactgaaaaatatccggttatatcattctgggtgtgttcttactgacaccaggggtccgctgccccatgtgtcctggtgagaaaatatatgcctggcacagcttttgtatagaaaattcttgagaagtaactgtccgctagaagtctgtccaaatttaaaatgtgtgccatattctggttcttgaaaataagattccagagctctttgatcgcttttaataaactgcaagttcattttaattgaagggccagcatatatacttgcaagataattttcagctgcaaggattcagcaccagttatgtttgaatgaaccctccttttctctgagattctggtccctggaaatccctttctgctagtggtgagcatgtaagtgttaagtttttaatctgggagcagggcataggaagaaaatgtcagtagtgctaatgcattttgcactagaacgcttcgggaaaatattcatgcttgccatctgttcatttctaaatttatattcataaagttacagtttgatacaggaattattaggagtaattcttttctgtttctgtttataatgaagaacactgtagctacattttcagaagttaacatcaagccatcaaacctgggtatagtgcagaagacgtggcacacactgaccacacattaggctgtgtcaccattgtgtggtgtacctgctggaagaattctagcatgctacttggggacataatttcagtgggaaatatgccactgaccgatttttttttttttcctctttgcagtggggctaggacagttgattcaacaaagtatttttttcttttttctcagtcctaatttggacaggtcaaagatgtgttcaggcattccaggtaacaggtgtgtatgtaaagttaaaaataggctttttaggaactcactctttagatatttacatccagcttctcatgttaaatatttgtccttaaagggtttgagatgtacatctttcatttcgtatttctcataggctatgccatgtgcggaattcaagttaccaatgtaacactggccagcgggcccagcaatctccatgtgtacttattacagtcttatttaaccaggggtcctaaccactaacattgtgactttgctttgagacctttcctctcctgggtactgaggtgctatgaagccaactgacaaagatgcatcacgtgtcttaggctgatgccactacccgatttgtttatttgcaatttgagccatttaaagaccaataaacttccttttttaaaaaaaaaaaaaaaaaaaaaaaaaaaa

SEQ ID NO: 74 - DNA, 3601 bp, Homo sapiens
gaccgtgaggccgagccgggagcgggcgtcttgccgaggcccgggcgggcgggagcaacggctacagacgccgcggggccaggtcgttgagggtcggcggcgggcgaggagcgcagggcgctcgggccgggggccgccggcgccatgggcaaccgcgggatggaagagctgatcccgctggtcaacaaactgcaggacgccttcagctccatcggccagagctgccacctggacctgccgcagatcgctgtagtgggcggccagagcgccggcaagagctcggtgctggagaacttcgtgggccgggacttccttccccgcggttcaggaatcgtcacccggcggcctctcattctgcagctcatcttctcaaaaacagaacatgccgagtttttgcactgcaagtccaaaaagtttacagactttgatgaagtccggcaggagattgaagcagagaccgacagggtcacggggaccaacaaaggcatctccccagtgcccatcaaccttcgagtctactcgccacacgtgttgaacttgaccctcatcgacctcccgggtatcaccaaggtgcctgtgggcgaccagcctccagacatcgagtaccgagtcaaggacatgatcctgcagttcatcagccgggagagcagcctcattctggctgtcacgcccgccaacatggacctggccaactccgacgccctcaagctggccaaggaagtcgatccccaaggcctacggaccatcggtgtcatcaccaagcttgacctgatggacgagggcaccgacgccagggacgtcttggagaacaagttgctcccgttgagaagaggctacattggcgtggtgaaccgcagccagaaggatattgagggcaagaaggacatccgtgcagcactggcagctgagaggaagttcttcctctcccacccggcctaccggcacatggccgaccgcatgggcacgccacatctgcagaagacgctgaatcagcaactgaccaaccacatccgggagtcgctgccggccctacgtagcaaactacagagccagctgctgtccctggagaaggaggtggaggagtacaagatctttcggcccgacgaccccacccctaaaaccaaagccctgctgcagatggtccagcagtttggggtggattttgagaagaggatcgagggctcaggagatcaggtggacactctggagctctccgggggcgcccgaatcaatcgcatcttccacgagcggttcccatttgagctggtgaagatggagtttgacgagaaggacttacgacgggagatcagctatgccattaagaacatccatggagtcaggaccgggcttttcaccccggacttggcattcgaggccattgtgaaaaagcaggtcgtcaagctgaaagagccctgtctgaaatgtgtcgacctggttatccaggagctaatcaatacagttaggcagtgtaccagtaagctcagttcctacccccggttgcgagaggagacagagcgaatcgtcaccacttacatccgggaacgggaggggagaacgaaggaccagattcttctgctgatcgacattgagcagtcctacatcaacacgaaccatgaggacttcatcgggtttgccaatgcccagcagaggagcacgcagctgaacaagaagagagccatccccaatcaggtgatccgcaggggctggctgaccatcaacaacatcagcctgatgaaaggcggctccaaggagtactggtttgtgctgactgccgagtcactgtcctggtacaaggatgaggaggagaaagagaagaagtacatgctgcctctggacaacctcaagatccgtgatgtggagaagggcttcatgtccaacaagcacgtcttcgccatcttcaacacggagcagagaaacgtctacaaggacctgcggcagatcgagctggcctgtgactcccaggaagacgtggacagctggaaggcctcgttcctccgagctggcgtctaccccgagaaggaccaggcagaaaacgaggatggggcccaggagaacaccttctccatggacccccaactggagcggcaggtggagaccattcgcaacctggtggactcatacgtggccatcatcaacaagtccatccgcgacctcatgccaaagaccatcatgcacctcatgatcaacaatacgaaggccttcatccaccacgagctgctggcctacctatactcctcggcagaccagagcagcctcatggaggagtcggctgaccaggcacagcggcgggacgacatgctgcgcatgtaccatgccctcaaggaggcgctcaacatcatcggtgacatcagcaccagcactgtgtccacgcctgtacccccgcctgtcgatgacacctggctccagagcgccagcagccacagccccactccacagcgccgaccggtgtccagcatacacccccctggccggcccccagcagtgaggggccccactccagggccccccctgattcctgttcccgtgggggcagcagcctccttctcggcgcccccaatcccatcccggcctggaccccagagcgtgtttgccaacagtgacctcttcccagccccgcctcagatcccatctcggccagttcggatccccccagggattcccccaggagtgcccagcagaagaccccctgctgcgcccagccggcccaccattatccgcccagccgagccatccctgctcgactaggcctcgaggggggcgtgctctcgggggggcctcacgcacccgcggcgcaggagcttcagtggtctggggccctccgccgcccctatgctgggaccaggctcccagtgggcagccctggcctcttggttaacgctggccccggtccagggccggcccctgtgcctggctggacaccgcactgcgcaaaggggccctggagctccaggcagggggcgctggggtgttgcactttgggggatggagtctcagggtggcagaggggggaccagaacccttgacaccatcctgaatgaggggtccagcctgggggggactctaccaaggtcttcttgggctgggaaagcccatgtagggcaggccttctataagtgcgggcaccaagggcgcctacatccccaggccttgctggggtgcaggggtatatcaacttcccattagcaggagctccccagcggcaagcctggcccagtgggctcggtagtgcccagctggcaggcctgaggtgtacatagtccttcccggccatattaaccacacagcctgagcctggcccagcctcggctgccagaggtgcctttgctaggcccggagccgttgcccggccttgcccttgccctattcctctcctcctcctcctcctgggtcccccagggtggctgggcttgggctatgtgggtggtggtggcggggggtcttgggggcctctcagctcccgcccatgcctccctgatgggtgggcccagggcggcctctctctgaggagacctcacccactcctcgctcagtttgaccactgtaagtgcctgcactctgtattctattaataaactaaaataaagggaagagcgtgctggtg

SEQ ID NO: 75 - protein, 232 aa, Homo sapiens
MESETEPEPVTLLVKSPNQRHRDLELSGDRGWSVGHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 76 - protein, 209 aa, Homo sapiens
MQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 77 - protein, 356 aa, Homo sapiens
GHLKAHLSRVYPERPRPEDQRLIYSGKLLLDHQCLRDLLPKEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 78 - protein, 391 aa, Homo sapiens
MESETEPEPVTLLVKSPNQRHRDLELSGDRGWSVGHLKAHLSRVYPERPRPEDQRLIYSGKLLLDHQCLRDLLPKQEKRHVLHLVCNVKSPSKMPEINAKVAESTEEPAGSNRGQYPEDSSSDGLRQREVLRNLSSPGWENISRPEAAQQAFQGLGPGFSGYTPYGWLQLSWFQQIYARQYYMQYLAATAASGAFVPPPSAQEIPVVSAPAPAPIHNQFPAENQPANQNAAPQVVVNPGANQNLRMNAQGGPIVEEDDEINRDWLDWTYSAATFSVFLSILYFYSSLSRFLMVMGATVVMYLHHVGWFPFRPRPVQNFPNDGPPPDVVNQDPNNNLQEGTDPETEDPNHLPPDRDVLDGEQTSPSFMSTAWLVFKTFFASLLPEGPPAIAN

SEQ ID NO: 79 - protein, 528 aa, Homo sapiens [sequence omitted]

SEQ ID NO: 80 - protein, 364 aa, Homo sapiens
RTRGCHLFSTYVASHKIEKEEQEKKRKEEEERRRREEEERERLQKEEEKRRREEEERLRREEEERRRIEEERLRLEQQKQQIMAALNSQTAVQFQQYAAQQYPGNYEQQQILIRQLQEQHYQQYMQQLYQVQLAQQQAALQKQQEVVVAGSSLPTSSKVNATVPSNMMSVNGQAKTHTDSSEKELEPEAAEEALENGPKESLPVIAAPSMWTRPQIKDFKEKIQQDADSVITVGRGEVVTVRVPTHEEGSYLFWEFATDNYDIGFGVYFEWTDSPNTAVSVHVSESSDDDEEEEENIGCEEKAKKNANKPLLDEIVPVYRRDCHEEVYAGSHQYPGRGVYLLKFDNSYSLWRSKSVYYRVYYTR

SEQ ID NO: 81 - protein, 528 aa, Homo sapiens
MAAVLNAERLEVSVDGLTLSPDPEERPGAEGAPLLPPPLPPPSPPGSGRGPGASGEQPEPGEAAAGGAAEEARRLEQRWGFGLEELYGLALRFFKEKDGKAFHPTYEEKLKLVALHKQVLMGPYNPDTCPEVGFFDVLGNDRRREWAALGNMSKEDAMVEFVKLLNRCCHLFSTYVASHKIEKEEQEKKRKEEEERRRREEEERERLQKEEEKRRREEEERLRREEEERRRIEEERLRLEQQKQQIMAALNSQTAVQFQQYAAQQYPGNYEQQQILIRQLQEQHYQQYMQQLYQVQLAQQQAALQKQQEVVVAGSSLPTSSKVNATVPSNMMSVNGQAKTHTDSSEKELEPEAAEEALENGPKESLPVIAAPSMWTRPQIKDFKEKIQQDADSVITVGRGEVVTVRVPTHEEGSYLFWEFATDNYDIGFGVYFEWTDSPNTAVSVHVSESSDDDEEEEENIGCEEKAKKNANKPLLDEIVPVYRRDCHEEVYAGSHQYPGRGVYLLKFDNSYSLWRSKSVYYRVYYTR

SEQ ID NO: 82 - protein, 528 aa, Homo sapiens [sequence omitted]

SEQ ID NO: 83 - protein, 300 aa, Homo sapiens
MRKHRRHDLPLEGAKVSSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 84 - protein, 250 aa, Homo sapiens
SDPVLMRKHRRHDLPLEGAKVSSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEA

SEQ ID NO: 85 - protein, 770 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSSLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSRDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGTYRI

SEQ ID NO: 86 - protein, 982 aa, Homo sapiens
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFGMPMLDLDDDDDREESLMMNRLANVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPPERPPPIPPDNRLSRHIHHVESVPSKDPPMPLEAWCPRDVFGTNQLVGCRLLGEGSPKPGITASSNVNGRHSRVGSDPVLMRKHRRHDLPLEGAKVFSNGHLGSEEYDVPPRLSPPPPVTTLLPSIKCTGPLANSLSEKTRDPVEEDDDEYKIPSSHPVSLNSQPSHCHNVKPPVRSCDNGHCMLNGTHGPSSEKKSNIPDLSIYLKGDVFDSASDPVPLPPARPPTRDNPKHGSSLNRTPSDYDLLIPPLGEDAFDALPPSLPPPPPPARHSLIEHSKPPGSSSRPSSGQDLFLLPSDPFVDLASGQVPLPPARRLPGENVKTNRTSQDYDQLPSCSDGSQAPARPPKPRPRRTAPEIHHRKPHGPEAALENVDAKIAKLMGEGYAFEEVKRALEIAQNNVEVARSILREFAFPPPVSPRLNL

SEQ ID NO: 87 - protein, 982 aa, Homo sapiens [sequence omitted]

SEQ ID NO: 88 - protein, 810 aa, Homo sapiens [sequence omitted]

SEQ ID NO: 89 - protein, 212 aa, Homo sapiens [sequence omitted]

SEQ ID NO: 90 - protein, 195 aa, Homo sapiens
MDPLFQQTHKQVHEIQSCMGRLETADKQSVHIVENEIQASIDQIFSRLERLEILSSKEPPNKRQNARLRVDQLKYDVQHLQTALRNFQHRRHAREQQERQREELLSRTFTTNDSDTTIPMDESLQFNSSLQKVHNGMDDLILDGHNILDGLRTQRLTLKVGSLLGDREKASCFSLIQQFSNCVYILITCPQIVIF

SEQ ID NO: 91 - protein, 213 aa, Homo sapiens
MDPLFQQTHKQVHEIQSCMGRLETADKQSVHIVENEIQASIDQIFSRLERLEILSSKEPPNKRQNARLRVDQLKYDVQHLQTALRNFQHRRHAREQQERQREELLSRTFTTNDSDTTIPMDESLQFNSSLQKVHNGMDDLILDGHNILDGLRTQRLTLKGTQKKILDIANMLGLSNTVMRLIEKRAFQDKYFMIGTQGSCQTAHFGGRSAGSS

SEQ ID NO: 92 - protein, 212 aa, Homo sapiens
MDPLFQQTHKQVHEIQSCMGRLETADKQSVHIVENEIQASIDQIFSRLERLEILSSKEPPNKRQNARLRVDQLKYDVQHLQTALRNFQHRRHAREQQERQREELLSRTFTTNDSDTTIPMDESLQFNSSLQKVHNGMDDLILDGHNILDGLRTQRLTLKGTQKKILDIANMLGLSNTVMRLIEKRAFQDKYFMIGGMLLTCVVMFLVVQYLT

SEQ ID NO: 93 - protein, 206 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDENVPFLLVGNKSDLEDKRQVSVEEAKNRAEQWNVNYVETSAKTRANVDKVFFDLMREIRARKMEDSKEKNGKKKRKSLAKRIRERCCIL

SEQ ID NO: 94 - protein, 209 aa, Homo sapiens
MVDYLANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDENVPFLLVGNKSDLEDKRQVSVEEAKNRAEQWNVNYVETSAKTRANVDKVFFDLMREIRARKMEDSKEKNGKKKRKSLAKRIRERCCIL

SEQ ID NO: 95 - protein, 870 aa, Homo sapiens
MGNRGMEELIPLVNKLQDAFSSIGQSCHLDLPQIAVVGGQSAGKSSVLENFVGRDFLPRGSGIVTRRPLILQLIFSKTEHAEFLHCKSKKFTDFDEVRQEIEAETDRVTGTNKGISPVPINLRVYSPHVLNLTLIDLPGITKVPVGDQPPDIEYRVKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPALRSKLQSQLLSLEKEVEEYKIFRPDDPTPKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELINTVRQCTSKLSSYPRLREETERIVTTYIREREGRTKDQILLLIDIEQSYINTNHEDFIGFANAQQRSTQLNKKRAIPNQGEILVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESADQAQRRDDMLRMYHALKEALNIIGDISTSTVSTPVPPPVDDTWLQSASSHSPTPQRRPVSSIHPPGRPPAVRGPTPGPPLIPVPVGAAASFSAPPIPSRPGPQSVFANSDLFPAPPQIPSRPVRIPPGIPPGVPSRRPPAAPSRPTIIRPAEPSLLD

SEQ ID NO: 96 - protein, 866 aa, Homo sapiens
MGNRGMEELIPLVNKLQDAFSSIGQSCHLDLPQIAVVGGQSAGKSSVLENFVGRDFLPRGSGIVTRRPLILQLIFSKTEHAEFLHCKSKKFTDFDEVRQEIEAETDRVTGTNKGISPVPINLRVYSPHVLNLTLIDLPGITKVPVGDQPPDIEYRVKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPALRSKLQSQLLSLEKEVEEYKIFRPDDPTPKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELINTVRQCTSKLSSYPRLREETERIVTTYIREREGRTKDQILLLIDIEQSYINTNHEDFIGFANAQQRSTQLNKKRAIPNQVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESADQAQRRDDMLRMYHALKEALNIIGDISTSTVSTPVPPPVDDTWLQSASSHSPTPQRRPVSSIHPPGRPPAVRGPTPGPPLIPVPVGAAASFSAPPIPSRPGPQSVFANSDLFPAPPQIPSRPVRIPPGIPPGVPSRRPPAAPSRPTIIRPAEPSLLD

SEQ ID NO: 97 - protein, 866 aa, Homo sapiens
MGNRGMEELIPLVNKLQDAFSSIGQSCHLDLPQIAVVGGQSAGKSSVLENFVGRDFLPRGSGIVTRRPLILQLIFSKTEHAEFLHCKSKKFTDFDEVRQEIEAETDRVTGTNKGISPVPINLRVYSPHVLNLTLIDLPGITKVPVGDQPPDIEYQIKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPALRSKLQSQLLSLEKEVEEYKIFRPDDPTPKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELINTVRQCTSKLSSYPRLREETERIVTTYIREREGRTKDQILLLIDIEQSYINTNHEDFIGFANAQQRSTQLNKKRAIPNQVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESADQAQRRDDMLRMYHALKEALNIIGDISTSTVSTPVPPPVDDTWLQSASSHSPTPQRRPVSSIHPPGRPPAVRGPTPGPPLIPVPVGAAASFSAPPIPSRPGPQSVFANSDLFPAPPQIPSRPVRIPPGIPPGVPSRRPPAAPSRPTIIRPAEPSLLD

SEQ ID NO: 98 - DNA, 3600 bp, Homo sapiens
ggtggagctgtcgcctagccgctatcgcagagtggagcggggctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatggagactttgctgttactgcttctctttgtgcagtgttagtattcaccctgggcagtgagtgccatgctttttggtgagggcagatcccagcacctattgaattaccatagagtaatgatgtaacagtgcaagattttttttttaagtgacataattgtccagttataagcgtatttagactgtggccatatatgctgtatttctttgtagaataaatggtttctcattaaactctaaagattagggaaaatggatatagaaaatcttagtatagtagaaagacatctgcctgtaattaaactagtttaagggtggaaaaatgcccatttttgctaattatcaatgggatatgattggttcagtttttttttttccagagttgttgtttgccaagctaatctgcctggttttatttatatcttgttattaatgtttcttctccaattctgaaatacttttgagtatggctatctatacctgccttttaagtttgaaactaactcatagattgcaaatattggttagtatttaactacatctgcctcggctcacaaattccgattagacctttatccagctagtgccaaataattgatcagatgctgaattgagaataagaatttgaggtctacattcttggttgttaatttagagcgtttggttaaagtatgtccttcagctgactccagtataatctcctctgctcattaaactgattccaggagattggatttgctgtgactagatacagatggagcaaatgtcctaacagagaaatagaggtgatgctgctaaagggagaaatgccaggcggacaaagttcagtgtcgggaattttccccgtgacattcactggggcatgagattttggaagaagttttttactttggtttagtctttttttccttcctttttattcagctagaatttctggtgggttgatggtagggtataatgtgtctgtgttgcttcaaattggtctgaaaggctatcctgcggaaagtcctgctttcctatctagcatttatttctctggcaaacttttctttcttttcttttttaaagtaaacttgtgtattgagtcttaactgtatttcagtattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattacgttcttacaagcttaaagcttgatttgatctttgtttaaatgccaaaatgtacttaaatgagttacttagaatgccataaaattgcagtttcatgtatgtatataatcatgctcatgtatatttagttacgtataatgctttctgagtgagttttactcttaaatcatttggttaaatcatttggcttgctgtttactcccttctgtagtttttaattaaaaactttaaagataagtctacattaaacaatgatcacatctaaagctttatctttgtgtaatctaagtatatgtgagaaatcagaattggcataatttgtcttagttgatattcaaggctttaaaagtcattattcctgggcttggtaagtgaatttatgagatttactgctctagaaagtatagatggcgaaaggaccgttttgtattgcttcctgattaccagtctgattataccatgtgtgctaatatactttttttgttatagattgtcttaatggtaggtcaagtaataaaaagagatgaaataatttaaaaaaaaaaaaaaaa

SEQ ID NO: 99 - DNA, 412 bp, Homo sapiens
agaggcgtcaagggaggccggagggagagtggggtggacagaggagcggagggacgagagggaagcgcacgatagctgcgcggagagagagcgaagagcaggaggaggaacaaaggcgacccaagacacccagagagggacagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaa

SEQ ID NO: 100 - DNA, 1940 bp, Homo sapiens
taattttcttgtgtgtttttaaaaattttgattatgctagtagttggctaatcagatcctcactccagtggtttgctctgtgacgttaggatactcccatgggatagaagttacgtatagggaatgtcagatattcttcattgtgctgacttgctttcgcttacagttgacttttgtgccctggtaattctgtatcctgtttaccgtttacctacttcccacgtcatcatgatttcttttgagggagaactgaatgaaattcccttaagggcctgacttcagcacccgtctctgcagaggttagtggctcatacttcctcccaggagctgaggttatcgactctcactgttgcctacagagcacagatcctgaactaaatgaaacatttacttggaataatgctaattctgtacatattttattccctagtccccacttccctgtttaaaaacaaaatctacttagaaaaaaatccctgtgaatcagttgtctaatgaatttagcaagttaaatgccagattgacattttgctttatagtttatacaagcatgtgtgtgtttttttctcgcagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatccgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatgg

SEQ ID NO: 101 - DNA, 1476 bp, Homo sapiens
ggcagagtggagcggggctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattaaagttgcttttattgcaccattttcaatttggagcattaactaaatgctcatacacagttaaataaatagaaagagttctatggaaaaaaaaaaaaa

SEQ ID NO: 102 - DNA, 3036 bp, Homo sapiens
gctgggagcaaagcgctgagggagctcggtacgccgccgcctcgcacccgcagcctcgcgcccgccgccgcccgtccccagagaaccatggagtctggcagtaccgccgccagtgaggaggcacgcagccttcgagaatgtgagctctacgtccagaagcataacattcaagcgctgctcaaagattctattgtgcagttgtgcactgctcgacctgagagacccatggcattcctcagggaatactttgagaggttggagaaggaggaggcaaaacagattcagaatctgcagaaagcaggcactcgtacagactcaagggaggatgagatttctcctcctccacccaacccagtggttaaaggtaggaggcgacgaggtgctatcagcgctgaggtctacacggaggaagatgcggcatcctatgttagaaaggttataccaaaagattacaagacaatggccgctttagccaaagccattgaaaagaatgtgctgttttcacatcttgatgataatgagagaagtgatatttttgatgccatgttttcggtctcctttatcgcaggagagactgtgattcagcaaggtgatgaaggggataacttctatgtgattgatcaaggagagacggatgtctatgttaacaatgaatgggcaaccagtgttggggaaggagggagctttggagaacttgctttgatttatggaacaccgagagcagccactgtcaaagcaaagacaaatgtgaaattgtggggcatcgaccgagacagctatagaagaatcctcatgggaagcacactgagaaagcggaagatgtatgaggaattccttagtaaagtctctattttagagtctctggacaagtgggaacgtcttacggtagctgatgcattggaaccagtgcagtttgaagatgggcagaagattgtggtgcagggagaaccaggggatgagttcttcattattttagaggggtcagctgctgtgctacaacgtcggtcagaaaatgaagagtttgttgaagtgggaagattggggccttctgattattttggtgaaattgcactactgatgaatcgtcctcgtgctgccacagttgttgctcgtggccccttgaagtgcgttaagctggaccgacctagatttgaacgtgttcttggcccatgctcagacatcctcaaacgaaacatccagcagtacaacagttttgtgtcactgtctgtctgaaatctgcctcctgtgcctcccttttctcctctccccaatccatgcttcactcatgcaaactgctttattttccctacttgcagcgccaagtggccactggcatcgcagcttcctgtctgtttatatattgaaagttgcttttattgcaccattttcaatttggagcattaactaaatgctcatacacagttaaataaatagaaagagttctatggagactttgctgttactgcttctctttgtgcagtgttagtattcaccctgggcagtgagtgccatgctttttggtgagggcagatccagcacctattgaattaccatagagtaatgatgtaacagtgcaagatttttttttttaagtgacataattgtccagttataagcgtatttagactgtggccatatatgctgtatttctttgtagaataaatggtttctcattaaactctaaagattagggaaatggatatagaaaatcttagtatagtagaaagacatctgcctgtaattaaactagtttaagggtggaaaaatgaaaatttttgctaattatcaatgggatatgattggttcagttttttttttccagagttgttgtttgccaagctaatctgcctggtttatttatatcttgttattaatgtttcttctccaattctgaaatacttttgagtatggctatctatacctgccttttaagtttgaaactaactcatagatgcaaatattggttagtatttaactacatctgcctcggctcacaaattccgattagacctttatccagctagtgccaaataattgatcagatgctgaattgagaataagaatttgaggtctacattcttggttgttaatttagagcgtttggttaaagtatgtccttcagctgactccagtataatctcctctgctcattaaactgattccaggagattggatttgctgtgactagatacagatggagcaaatgtcctaacagagaaatagaggtgatgctgctaaagggagaaatgccaggcggacaaagttcagtgtcgggaattttccccgtgacattcactggggcatgagattttggaagaagttttttactttggtttagtctttttttcctcctttttattcagctagaatttctggtgggttgatggtagggtataatgtgtctgtgttgcttcaaattggtctgaaaggctatcctgctgaaagtcctgctttcctatctagcatttattcctctggcaaacttttctttcttttcttttttaaagtaaacttgtgtattgagtcttaactgtatttcagtattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattatgttcttacaagcttaaagcttgatttgatct

SEQ ID NO: 103 - DNA, 3036 bp, Homo sapiens; identical to SEQ ID NO: 102

SEQ ID NO: 104 - DNA, 1010 bp, Homo sapiens
tattttccagccttatgtgttacattattccaatgatacccaacagtttatttttattatttttttaaacaaaatttcacagttctgtaatgtaggcacttttattttcattgtgatttatatataaggtaatgtagggttatatttgggagtgactgcaagcatttttccatctgtgtgcaactaactgactctgttattgatcccttctcctgccctttcccaggtaatttaaattggtcatggtagatttttttcatagatttgaaaaacttttaggttgttaccaagtatgaagtataaatctggggaagaggttttatttacattttagggtgggtaagaaagccaccttgttacaaattttttaatttccaaaataatctatattaaatgagggtttctgatctgtactttgtgtttagctacctttttatatttaaaaaattaaaaatgaaaattacgttcttacaagcttaaagcttgatttgatctttgtttaaatgccaaaatgtacttaaatgagttacttagaatgccataaaattgcagtttcatgtatgtatataatcatgctcatgtatatttagttacgtataatgctttctgagtgagttttactcttaaatcatttggttaaatcatttggcttgctgtttactcccttctgtagtttttaattaaaaactttaaagataagtctacattaaacaatgatcacatctaaagctttatctttgtgtaatctaagtatatgtgagaaatcagaattggcataatttgtcttagttgatattcaaggctttaaaagtcattattcctgggcttggtaagtgaatttatgagatttactgctctagaaagtatagatggccaaaggaccgttatgtattgcttcctgattaccagtctgattataccatgtgtgctaatatactttttttgttatagattgtcttaatggtaggtcaagtaataaaaagagatgaaataatttaaaaaaaaaaaaa

SEQ ID NO: 105 - DNA, 2685 bp, Homo sapiens
tcgggctgaggttcccgggcgggcgggcgcggagagacgcgggaagcaggggctgggcgggggtcgcggcgccgcagctagcgcagccagcccgagggccgccgccgccgccgcccagcgcgctccggggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagttttgtttaaaaaaaaaaaaaaaaaa

SEQ ID NO: 106 - DNA, 120 bp, Homo sapiens
ggtgccctgagaacaggactgagtgatggcttccaactccagcgatgtgaaagaattcttagccaaagccaaagaagattttcttaaaaaatgggaaagtcccgctcagaacacagccca

SEQ ID NO: 107 - DNA, 2549 bp, Homo sapiens
  misc_feature 6: n = A,T,C or G
cagtgngctccgggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagtttgtt

SEQ ID NO: 108 - DNA, 936 bp, Homo sapiens
gaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaagtaggagcctccccagccctccccttcccctgaggccggctctgctctcctgctctcgcctcctcctcaccctgtgcccccccatcttgctccagggctacaacaaggccgtggactggtgggccctgggggttcttatctatgaaatggccgctggctacccgcccttcttcgcagaccagcccatccagatctatgagaagatcgtctctgggaaggtgaggtccggatgtgggacacagccctggaagaaacagaccgttccctgctcacccatcctattccctggggagccctgcttgttgtcagaataatctagaagttccttaaaaaaaaaaaaaaaaa

SEQ ID NO: 109 - DNA, 560 bp, Homo sapiens
tgagaacaggactgagtgatggcttccaactccagcgatgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgc

SEQ ID NO: 110 - DNA, 594 bp, Homo sapiens
cccagtggcctctgggttgggtttctcttcctgctcccaccccacggctccctagctccccctgcaggcagggttctggggacagacagccgaacagacacggcaggtctcatgagccttcccagccaccgtagtgccggtgccctgagaacaggactgagtgatggcttccaactccagcgatgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgt

SEQ ID NO: 111 - DNA, 4535 bp, Homo sapiens
agcgggtctgcccgccgccgccactgctgctgccaccgccgtcgccgccgccgccgccgccgccactgctgctgccggtgctaaggagttcgctggagccctttcctcagacccggcccggtcttcgcgcccggactcctggcgccagcgctaggcgcactcaccgctctgacgggtgcagacgcgggagttgtcccagactgtggagtggcgggcacggccccagctccccttccgttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctcaattcactagacatcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatccaacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatcccgtgtgcgcactctgcatccacctatgtaacaaggcaccgctaagcaagcattgtctgtgccataacacagtactagaccactttcttacttctctttgggttgtctttctcctctcctatatccatttcttccttttccaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaactgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgcaagaaaaaaataaaatactctgctctagcaagttttgtgtaacaaaggcatatcgtcatgttaataaatttaaaacatcattcgtataaaatattttaattttcttgtatttcatttagacccaagaacatgctgaccaatgtgttctatatgtaaactacaaattctatggtagctttgttgtatattattgtaaaattattttaataagtcatggggatgacaatttgattattacaatttagttttcagtaatcaaaaagatttctatgaattctaaaaaatatttttttctatgaaattactagtgcccagctgtagaatctaccttaggtagatgatccctagacatacgttggttttgagggctattcagccattccattttactctctatttaaaggccgtgagcaagcttgtcatgagcaaatatgtcaagggagtcaatctctgaccaatcaagtacactaaattagaatatttttaaagtatgtaacattcccagtttcagccacaatttagccaagaataagataaaaacttgaataagaagtaagtagcataaatcagtatttaacctaaaattacatatttgaaacagaagatattatgttatgctcagtaaataattaagagatggcattgtgtaagaaggagccctagactgaaagtcaagacatctgaatttcaggctggaaaactatcagtatgatctcagcctcagttctcttgtctgtaagatggaagaactggattaggcagtttgtaagattcctcctaactttcacagtcgatgacaagattgtctttttatctgatattttgaagggtatattgctttgaagtaagtctcaataaggcaatatattttagggcatctttcttcttatctctgacagtgttcttaaaattatttgaatatcataagagccttggtgtctgtcctaattcctttctcactcaccgatgctgaatacccagttgaatcaaactgtcaacctaccaaaaacgatattgtggcttatgggtattgctgtctcattcttggtatattcttgtgttaactgcccattggcctgaaaatactcattgtaagcctgaaaaaaaaaatctttcccactgttttttctgcttgttgtaagaatcaaatgaaataatgtatgtgaaagcaccttgtaaactgtaacctatcaatgtaaaatgttaaggtgtgttgttatttcattaattacttctttgtttagaatggaatttcctatgcactactgtagctaggaaatgctgaaaacaactgtgttttttaattaatcaataactgcaaaattaaagtaccttcaatggataagacaacaaaaaaaaaaaaaaaa

SEQ ID NO: 112 - DNA, 296 bp, Homo sapiens
aaaaaaaatctttcccactgttttttctgcttgttgtaagaatcaaatgaaataatgtatgtgaaagcaccttgtaaactgtaacctatcaatgtaaaatgttaaggtgtgttgttatttcattaattacttctttgtttagaatggaatttcctatgcactactgtagctaggaaatgctgaaaacaactgtgttttttaattaatcaataactgcaaaattaaagtaccttcaatggataagacaaaaaaaaaaaaaaaaaaaaaaaaaaaaaaaaaaaaaaaaaaaaaaaaaa

SEQ ID NO: 113 - DNA, 2850 bp, Homo sapiens
gttattttgagcaatatgttttggaaaggttggttttcatcatgagtgcacgcaaatcatcagatgcatctgcttgctcctcttcagaaatatctgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatcccgtgtgcgcactctgcatccacctatgtaacaaggcaccgctaagcaagcattgtctgtgccataacacagtactagaccactttcttacttctctttgggttgtctttctcctctcctacatccatttcttccttttccaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaactgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 114 - DNA, 1951 bp, Homo sapiens
gttcgctggagccctttcctcagacccggcccggtcttcgcgcccggactcctggcgccagcgctaggcgcactcaccgctctgacgggtgcagacgcgggagttgtcccagactgtggagtggcgggcacggccccagccccccttcccttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaagaacttttgatatgaacaaaacaaaactttgagaaaaattaacagacaaggcagtgatttatttttgaagaatttgagaagtgtagactctcaagaggactaaaggtcatatgaagaatgatgagagaaccaaaatacattaaaatcacaaatggaagaagaatattttactaatacaaaaactaagaatgtaaatgttataataattgtttcaaatcatttaattgacagtaattataaagttcttgaatctttactatattacttttatttatacttcatataagaaatccagttttctaacaaggatactgtcataactaaatttacatttattaagaaaaactgctttagttaaaattaatgtgtcttcatttttatgcattggcctcgatttgccaatcattctctattggttaaaatttatattcagctgtttatgaatatatattcattttatatcaaactttaaaattttgtatctaataatcagcatatattctaaaatcataacagtctaaatcctgggcaccttagaagaatgacaccagaaaaccttattatatcacaatattctgttttccccttcatttatttagaaatatgacaggatatttggtgtacttttgttttttaactaaaagtaccagattctctctccccatgtgggatataaaattatccccatctcttactccctttactcatctaaagtagaagtcatgaaagtggaatttttgccattaaaaggctctgtattatgtgaagttagattgtattaaccatttcccaataaatcatctgtttcaaaactcaaattcaaactagaatgtgtctctattcacattgcaaaaatattattgtctctctggttagtggctaaaagccaaattggaaactaactagttttttaaattttttaaattgtgcaaattattaaaaatccaatttggtcttataaaaaaaaaaaaaaaaaaaaaaaaaaaaaaaaaaaaaaaaaaaaaa

SEQ ID NO: 115 - DNA, 2945 bp, Homo sapiens
ccagccccccttcccttccctgaccccttcttgccatcgccccagacatggggaacgcggcgaccgccaagaaaggcagcgaggtggagagcgtgaaagagtttctagccaaagccaaagaagactttttgaaaaaatgggagaatccaactcagaataatgccggacttgaagattttgaaaggaaaaaaacccttggaacaggttcatttggaagagtcatgttggtaaaacacaaagccactgaacagtattatgccatgaagatcttagataagcagaaggttgttaaactgaagcaaatagagcatactttgaatgagaaaagaatattacaggcagtgaattttcctttccttgttcgactggagtatgcttttaaggataattctaatttatacatggttatggaatatgtccctgggggtgaaatgttttcacatctaagaagaattggaaggttcagtgagccccatgcacggttctatgcagctcagatagtgctaacattcgagtacctccattcactagacctcatctacagagatctaaaacctgaaaatctcttaattgaccatcaaggctatatccaggtcacagactttgggtttgccaaaagagttaaaggcagaacttggacattatgtggaactccagagtatttggctccagaaataattctcagcaagggctacaataaggcagtggattggtgggcattaggagtgctaatctatgaaatggcagctggctatcccccattctttgcagaccaaccaattcagatttatgaaaagattgtttctggaaaggtccgattcccatcccacttcagttcagatctcaaggaccttctacggaacctgctgcaggtggatttgaccaagagatttggaaatctaaagaatggtgtcagtgatataaaaactcacaagtggtttgccacgacagattggattgctatttaccagaggaaggttgaagctccattcataccaaagtttagaggctctggagataccagcaactttgatgactatgaagaagaagatatccgtgtctctataacagaaaaatgtgcaaaagaatttggtgaattttaaagaggaacaagatgacatctgagctcacactcagtgtttgcactctgttgagagataaggtagagctgagaccgtccttgttgaagcagttacctagttccttcattccaacgactgagtgaggtctttattgccatcatccgtgtgcgcactctgcatccacctatgtaacaaggcaccgctaagcaagcattgtctgtgccataacacagtactagaccactttcttacttctctttgggttgtctttctcctctcctacatccatttcttccttttcaatttcattggttttctctaaacagtgctccattttattttgttggtgtttcagatgggcagtgttatggctacgtgatatttgaagggaaggataagtgttgctttcagtagttattgccaatattgttgttggtcaatggcttgaagataaactttctaataattattatttctttgagtagctcagacttggttttgccaaaactcttggtaatttttgaagatagactgtcttatcaccaaggaaatttatacaaattaagactaactttcttggaattcactattctggcaataaattttggtagactaatacagtacagctagacccagaaatttggaaggctgtagatcagaggttctagttccctttccctccttttatatcctcctctccttgagtaatgaagtgaccagcctgtgtagtgtgacaaacgtgtctcattcagcaggaaaaactaatgatatggatcatcacccagattctctcacttggtaccagcatttctgtaggtattagagaagagttctaagttttctaaaccttaactgttccttaaggattttagccagtattttaatagaacatgattaatgaaagtgacaaattttaaattttctctaatagtcctcatcataaactttttaaaggaaaataagcaaactaaaaagaacattggtttagataaatacttatactttgcaaagtcaaaaatggcttgatttttggaaacaatatagaggtattcatatttaaatgagggtttacatttgttttgttttgtaaccgttaaaaagaagttgtttccagctaattattgtggtgtactatatttgtgagcctagggtaggggcactgctgcaacttctgctttcatcccatgcctcatcaatgaggaaagggaacaaagtgtataaaacctgccacaattgtattttaattttgaggtatgatattttcagatatttcataatttctaacctctgttctctcagtaaacagaatgtctgatcgatcatgcagatacaatgttggtatttgagaggttagtttttttcctacacttttttttgccaactgacttaacaacattgctgtcaggtggaaatttcaagcacttttgcacatttagttcagtgtttgttgagaatccatggcttaacccacttgttttgctatttttttctttgcttttaattttccccatctgattttatctctgcgtttcagtgacctaccttaaaacaacacacgagaagagttaaactgggttcattttaatgatcaatttacctgcatataaaatttatttttaatcaagctgatcttaatgtatataatcattctatttgctttattatcggtgcaggtaggtcattaacaccacttcttttcatctgtaccacaccctggtgaaacctttgaagacataaaaaaaacctgtctgagatgttctttctaccaatctatatgtctttcggttatcaagtgtttctgcatggtaatgtcatgtaaatgctgatattgatttcactggtccatctatatttaaaacgtgc

SEQ ID NO: 116 - protein, 87 aa, Homo sapiens
MESGSTAASEEARSLRECELYVQKHNIQALLKDSIVQLCTARPERPMAFLREYFERLEKEEAKQIQNLQKAGTRTDSREDEISPPPP

SEQ ID NO: 117 - protein, 381 aa, Homo sapiens
MESGSTAASEEARSLRECELYVQKHNIQALLKDSIVQLCTARPERPMAFLREYFERLEKEEAKQIQNLQKAGTRTDSREDEISPPPPNPVVKGRRRRGAISAEVYTEEDAASYVRKVIPKDYKTMAALAKAIEKNVLFSHLDDNERSDIFDAMFSVSFIAGETVIQQGDEGDNFYVIDQGETDVYVNNEWATSVGEGGSFGELALIYGTPRAATVKAKTNVKLWGIDRDSYRRILMGSTLRKRKMYEEFLSKVSILESLDKWERLTVADALEPVQFEDGQKIVVQGEPGDEFFIILEGSAAVLQRRSENEEFVEVGRLGPSDYFGEIALLMNRPRAATVVARGPLKCVKLDRPRFERVLGPCSDILKRNIQQYNSFVSLSV

SEQ ID NO: 118 - protein, 207 aa, Homo sapiens
EFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKVGASPALPFP

SEQ ID NO: 119 - protein, 181 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFA

SEQ ID NO: 120 - protein, 144 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIV

SEQ ID NO: 121 - protein, 31 aa, Homo sapiens
MASNSSDVKEFLAKAKEDFLKKWESPAQNTA

SEQ ID NO: 122 - protein, 351 aa, Homo sapiens
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 123 - protein, 351 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLHSLDLIYRDLKPENLLIDHQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVSDIKTHKWFATTDWIAIYQRKVEAPFIPKFRGSGDTSNFDDYEEEDIRVSITEKCAKEFGEF

SEQ ID NO: 124 - protein, 257 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLHSLDLIYRDLKPENLLIDHQGYIQVTDFGFAKRVKGRTWTLCGTPEYLAPEIILSKGYNKAVDWWALGVLIYEMAAGYPPFFADQPIQIYEKIVSGKNF

SEQ ID NO: 125 - protein, 351 aa, Homo sapiens
MGNAATAKKGSEVESVKEFLAKAKEDFLKKWENPTQNNAGLEDFERKKTLGTGSFGRVMLVKHKATEQYYAMKILDKQKVVKLKQIEHTLNEKRILQAVNFPFLVRLEYAFKDNSNLYMVMEYVPGGEMFSHLRRIGRFSEPHARFYAAQIVLTFEYLNSLDIIYRDLKPENLLIDHQGYIQVTDFGFAKRVKGRTWTLCGTPEYLAPEIILSKGYNKAVDWWALGVLIYEMAAGYPPFFADQPIQIYEKIVSGKVRFPSNFSSDLKDLLRNLLQVDLTKRFGNLKNGVSDIKTHKWFATTDWIAIYQRKVEAPFIPKFRGSGDTSNFDDYEEEDIRVSITEKCAKEFGEF

SEQ ID NO: 126 - DNA, 53 bp, Artificial Sequence
  Scrambled control oligonucleotide
cacacactgccgtcaactgttcaagagacagttgacggcagtgtgtgtttttt

SEQ ID NO: 127 - DNA, 61 bp, Artificial Sequence
  Scrambled control oligonucleotide
aattaaaaaacacacactgccgtcaactgtctcttgaacagttgacggcagtgtgtgggcc

SEQ ID NO: 128 - DNA, 50 bp, Artificial Sequence
  Posh RNAi
aacagaggccttggaaacctggaagcttgcaggtttccaaggcctctgtt

SEQ ID NO: 129 - DNA, 54 bp, Artificial Sequence
  Posh RNAi
gatcaacagaggccttggaaacctgcaagcttccaggtttccaaggcctctgtt

SEQ ID NO: 130 - DNA, 24 bp, Artificial Sequence
  Primer
actagtcaaggtcgggcaggaaga

SEQ ID NO: 131 - DNA, 48 bp, Artificial Sequence
  Primer
gccgaattcaaaaaggatccggcgatatccggtgtttcgtcctttcca

SEQ ID NO: 132 - DNA, 23 bp, Artificial Sequence
  siRNA
gggaaguucuucggaaccudtdt

SEQ ID NO: 133 - DNA, 23 bp, Artificial Sequence
  siRNA
dtdtcccuucaagaagccuugga

SEQ ID NO: 134 - protein, 836 aa, Homo sapiens
RTLVGSGVEELPSNILLVRLLDGIKQRPWKPGPGGGSGTNCTNALRSQSSTVANCSSKDLQSSQGGQQPRVQSWSPPVRGIPQLPCAKALYNYEGKEPGDLKFSKGDIIILRRQVDENWYHGEVNGIHGFFPTNFVQIIKPLPQPPPQCKALYDFEVKDKEADKDCLPFAKDDVLTVIRRVDENWAEGMLADKIGIFPISYVEFNSAAKQLIEWDKPPVPGVDAGECSSAAAQSSTAPKHSDTKKNTKKRHSFTSLTMANKSSQASQNRHSMEISPPVLISSSNPTAAARISELSGLSCSAPSQVHISTTGLIVTPPPSSPVTTGPSFTFPSDVPYQAALGTLNPPLPPPPLLAATVLASTPPGATAAAAAAGMGPRPMAGSTDQIAHLRPQTRPSVYVAIYPYTPRKEDELELRKGEMFLVFERCQDGWFKGTSMHTSKIGVFPGNYVAPVTRAVTNASQAKVPMSTAGQTSRGVTMVSPSTAGGPAQKLQGNGVAGSPSVVPAAVVSAAHIQTSPQAKVLLHMTGQMTVNQARNAVRTVAAHNQERPTAAVTPIQVQNAAGLSPASVGLSHHSLASPQPAPLMPGSATHTAAISISRASAPLACAAAAPLTSPSITSASLEAEPSGRIVTVLPGLPTSPDSASSACGNSSATKPDKDSKKEKKGLLKLLSGASTKRKPRVSPPASPTLEVELGSAELPLQGAVGPELPPGGGHGRAGSCPVDGDGPVTTAVAGAALAQDAFHRKASSLDSAVPIAPPPRQACSSLGPVLNESRPVVCERHRVVVSYPPQSEAELELKEGDIVFVHKKREDGWFKGTLQRNGKTGLFPGSFVENI

SEQ ID NO: 135 - DNA, 1857 bp, Rat
aagacaccaagtgtcgttgtggggtcgcagacggctgcgtcgccgcccgttcggcatccctgagcgcagtcgagcctccagcgccgcagacatggagcccgagccacagcccgagccggtcacgctgctggtgaagagccccaatcagcgccaccgcgacttggagctgagtggcgaccgcggttggagtgtgagtcgcctcaaggcccacctgagccgagtctaccccgaacgcccgcgcccagaggaccagaggttaatttattctgggaagctgctgttggatcaccaatgtctccaagacttgcttccaaagcaggaaaagcgacatgttttgcacctcgtgtgcaatgtgaggagtccctcaaaaaagccagaagccagcacaaagggtgctgagtccacagagcagccggacaacactagtcaggcacagtatcctggggattcctcaagcgatggcttacgggaaagggaagtccttcggaaccttcctccctctggatgggagaacgtctctaggcctgaagccgtccagcagactttccaaggcctcgggcccggcttctctggctacaccacctacgggtggctgcagctctcctggttccagcagatctatgcaagacagtactacatgcaatacttggctgccactgctgcttcaggagcttttggccctacaccaagtgcacaagaaatacctgtggtctctacaccggctcccgcccctatacacaaccagtttccggcagaaaaccagccggccaatcagaatgcagccgctcaagcggttgttaatcccggagccaatcagaacttgcggatgaatgcacaaggcggccctctggtggaagaagatgatgagataaaccgagactggttggattggacctactcagcagcgacattttccgttttcctcagcattctttacttctactcctccctgagcagattcctcatggtcatgggcgccaccgtagtcatgtacctgcaccacgtcgggtggtttccattcagacagaggccagttcagaacttcccagatgacggtccccctcaggaagctgccaaccaggaccccaacaataacctccagggaggtttggaccctgaaatggaagaccccaaccgcctccccgtaggccgtgaagtgctggaccctgagcataccagcccctcgttcatgagcacagcatggctagtcttcaagactttctttgcctctcttcttccggaaggcccaccagccctagcaaactgatggcccctgtgctctgttgctggaggctttcacagcttggactggatcgtcccctggcgtggactcgagagagtcattgaaaacccacaggatgacgatgtgcttctgtgccaagcaaaagcacaaactaagacatgaagccgtggtacaaactgaacagggcccctcatgtcgttattctgaagagctttaatgtatactgtatgtagtctcataggcactgtaaacagaaggcccagggtcgcatgttctgcctgagcacctccccagacgtgtgtgcatgtgtgccgtacatggaagtcatagacgtgtgtgcatgtgtgctctacatggaagtcatagatgcagaaacggttctgctggttcgatttgattcctgttggaatgttgcaattacactaagtgtactactttatataatcagtgacttgctagacatgttagcaggacttttctaggagagacttattgtatcattgctttttaaaacgcagtgcttacttactgagggcggcgacttggcacaggtaaagcctttgccgggttttctgttcaataaagttttgctatgaacgacaaaaaaaaaaaaa

SEQ ID NO: 136 - protein, 391 aa, Rat
MEPEPQPEPVTLLVKSPNQRHRDLELSGDRGWSVSRLKAHLSRVYPERPRPEDQRLIYSGKLLLDHQCLQDLLPKQEKRHVLHLVCNVRSPSKKPEASTKGAESTEQPDNTSQAQYPGDSSSDGLREREVLRNLPPSGWENVSRPEAVQQTFQGLGPGFSGYTTYGWLQLSWFQQIYARQYYMQYLAATAASGAFGPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGPPQEAANQDPNNNLQGGLDPEMEDPNRLPVGREVLDPEHTSPSFMSTAWLVFKTFFASLLPEGPPALAN

SEQ ID NO: 137 - DNA, 1871 bp, Mus musculus
aaagacgccaagtgtcgttgtgtggtctcagacggctgcgtcgccgcccgttcggcatccctgagcgcagtcgagccgccagcgacgcagacatggagcccgagccacagcccgagccggtcacgctgctggtgaagagtcccaatcagcgccaccgcgacttggagctgagtggcgaccgcagttggagtgtgagtcgcctcaaggcccacctgagccgagtctaccccgagcgcccgcgtccagaggaccagaggttaatttattctgggaagctgctgttggatcaccagtgtctccaagatttgcttccaaagcaggaaaagcgacatgttttgcaccttgtgtgcaatgtgaagaatccctccaaaatgccagaaaccagcacaaagggtgctgaatccacagagcagccggacaactctaatcagacacagcatcctggggactcctcaagtgatggtttacggcaaagagaagttcttcggaacctttctccctccggatgggagaacatctctaggcctgaggctgtccagcagactttccaaggcctggggcctggcttctctggctacacaacgtatgggtggctgcagctctcctggttccagcagatctatgcaaggcagtactacatgcaatacttagctgccactgctgcatcaggaacttttgtcccgacaccaagtgcacaagagatacctgtggtctctacacctgctccggctcctatacacaaccagtttccggcagaaaaccagccggccaatcagaatgcagctgctcaagcggttgtcaatcccggagccaatcagaacttgcggatgaatgcacaaggtggccccctggtggaggaagatgatgagataaaccgagactggttggattggacctattccgcagcgacgttttctgttttcctcagcatcctttacttctactcctcgctgagcagatttctcatggtcatgggtgccactgtagtcatgtacctgcaccacgtcgggtggtttccgttcagacagaggccagttcagaacttcccggatgatggtggtcctcgagatgctgccaaccaggaccccaacaataacctccagggaggtatggacccagaaatggaagaccccaaccgcctccccccagaccgcgaagtgctggaccctgagcacaccagcccctcgtttatgagcacagcatggctagtcttcaagactttctttgcctctcttcttccagaaggcccaccagccctagccaactgatggcccttgtgctctgtcgctggtggctttgacagctcggactggatcgtctggctccggctccttttcctcccctggcgtggactcgacagagtcattgaaaacccacaggatgacatgtgcttctgtgccaagcaaaagcacaaactaagacatgaagccgtggtacaaactgaacagggcccctcatgtcgttattctgaagagctttaatgtatactgtatgtagtttcataggcactgtaagcagaaggcccagggtcgcatgttctgcctgagcacctccccagatgtgtgtgcatgtgtgctgtacatggaagtcatagacgtgtgtgcatgtgtgctctacatggaagtcatagatgcagaaacggttctgctggttcgatttgattcctgttggaatgttcaaattacactaagtgtactactttatataatcagtgaattgctagacatgttagcaggacttttctaggagagacttatgtataattgctttttaaaatgcagtgctttcctttaaaccgagggtggcgacttggcagaggtaaaacctttgccgagttttctgttcaataaagttttgctatgaatgactgt

SEQ ID NO: 138 - protein, 391 aa, Mus musculus
MEPEPQPEPVTLLVKSPNQRHRDLELSGDRSWSVSRLKAHLSRVYPERPRPEDQRLIYSGKLLLDHQCLQDLLPKQEKRHVLHLVCNVKNPSKMPETSTKGAESTEQPDNSNQTQHPGDSSSDGLRQREVLRNLSPSGWENISRPEAVQQTFQGLGPGFSGYTTYGWLQLSWFQQIYARQYYMQYLAATAASGTFVPTPSAQEIPVVSTPAPAPIHNQFPAENQPANQNAAAQAVVNPGANQNLRMNAQGGPLVEEDDEINRDWLDWTYSAATFSVFLSILYFYSSLSRFLMVMGATVVMYLHHVGWFPFRQRPVQNFPDDGGPRDAANQDPNNNLQGGMDPEMEDPNRLPPDREVLDPEHTSPSFMSTAWLVFKTFFASLLPEGPPALAN

SEQ ID NO: 139 - DNA, 849 bp, Rat
caagacccacactgagaatcccgagaaagtccttgagccagaagctgcggaagaggccttggaaaatggacccaaagactctcttccagtgattgcagctccttccatgtggacaagaccacagatcaaagactttaaagagaagattcggcaggatgcagattctgtgattacagtccggcgaggagaagtagtcaccgttcgagtccccactcatgaggaaggatcatacctcttttgggaatttgccacagacagttatgacattgggtttggggtttattttgaatggacagactctccaaacgctgctgtcagtgtgcatgtcagtgagtccagtgacgacgaagaggaggaggaagaaggtaggccttggttcacatctagtcctgctaacactcatagacaggaagtgctctgttcaggattgggatagtgggtgaaaagactctgagaaaggcctgcctgtcacaaaggccctaaacatgaatttctctaaaatgccaagaaagtctgtttggttctgagtgttgttttacttcagtctagtgtgttgggaagctaagtatgtgaagacccttagaggagcggagttgtttgtatattttgtttttgagacaggaacttactttcttgtccaggctggtcttgaactggtgaactcaaaacagccctcttcctcaccctccctagtagctgggactactggtgtatgccaccatgccccagtttgtaaaggtttgaaagagcaagtaagaattcttgccaacaaaagaatttatatttccgataacaacgtttttgtttgaaagacaggatccctctgttaatttgaggacagcctggtct

SEQ ID NO: 140 - protein, 109 aa, Rat
MWTRPQIKDFKEKIRQDADSVITVRRGEVVTVRVPTHEEGSYLFWEFATDSYDIGFGVYFEWTDSPNAAVSVHVSESSDDEEEEEEGRPWFTSSPANTHRQEVLCSGLG

SEQ ID NO: 141 - DNA, 1724 bp, Mus musculus
gagcaggaagtcgatacgtggccgccgcctgtccccgccgaggaggcgctgcgagcggagatggcggcgcagctgaacgtagagcagctcgaggtgtcgctcgacggtctcacgctgagcccggactcggaggagaggcccggcgcggagggggccccgccgcagacgccgccgtcctcggccccggggaatggactgggctccggcgcctcggggcagcagcgggagcccggggaggcggcggctgagggcgcggccgaggaggcgcgacggatggagcagcactggggctttggcctggaggagctttacggcctggcgctgcgcttctacaaaataaaagatggcaaagcctttcatccaacttatgaagaaaaactgaagttcgtggcactgcataagcaagttcttttgggcccatataacccagacacgtcccctgaggttggattctttgatgtgttggggaatgataggaggagagaatgggcagctctgggaaacatgtccaaggaggatgccatggtagagtttgtgaagcttctaaataagtgttgtcctctcctctcggcatatgttgcgtcccacagaatagagaaggaagaagaagagaaaagaagaaaggcggaggaggagcgaaggcagcgtgaagaggaagaacgagagcggctgcaaaaggaagaagagaagcggaagcgagaggaggaagaccggctgagacgggaggaggaagagaggcggcggatagaggaagagaggcttcggctggaacagcaaaagcagcagataatggcagctttaaactcgcagactgccgtgcaattccagcagtatgcagcccagcagtatccagggaactacgaacaacagcagattctcatccgccagctgcaggagcagcactatcagcagtatatgcagcagttatatcaagtccagcttgcacaacaacaggcagcattacagaaacagcaagaagtagtgatggctggggcatcattgcctgcatcatcaaaggtgaacacagctggagcaagtgatacactgtcagttaatggacaggccaaaacccacactgaaaattccgaaaaagtccttgagccagaagctgcagaagaagccttggaaaatggaccaaaagactctcttccagtgattgcagctccatccatgtggacaagaccacaaatcaaagactttaaagagaagattcggcaggatgcagattctgtgattacagtacgtcgaggagaagtcgtcaccgtccgagtcccgactcatgaggaaggatcatacctattttgggaatttgccacagacagttatgacattgggtttggggtttattttgaatggacagactctccaaatgctgctgtcagtgtgcatgtcagtgagtccagtgacgaggaggaggaggaggaagaaaatgtcacttgtgaagaaaaagcaaaaaagaacgccaacaagcctctgctggatgagattgtacctgtgtaccggcgggactgtcacgaggaagtatatgcaggcagccaccagtatccagggaggggagtctatctcctcaagtttgataattcctactctctgtggaggtccaagtccgtctactacagagtctattatactagatagagctgctgttccagggtcgggagtctagggttgagcacaacatgacgtttaatttcctttgaaaaaaaaaaaaaaaaaaaaaaaaa

SEQ ID NO: 142 - protein, 525 aa, Mus musculus
MAAQLNVEQLEVSLDGLTLSPDSEERPGAEGAPPQTPPSSAPGNGLGSGASGQQREPGEAAAEGAAEEARRMEQHWGFGLEELYGLALRFYKIKDGKAFHPTYEEKLKFVALHKQVLLGPYNPDTSPEVGFFDVLGNDRRREWAALGNMSKEDAMVEFVKLLNKCCPLLSAYVASHRIEKEEEEKRRKAEEERRQREEEERERLQKEEEKRKREEEDRLRREEEERRRIEEERLRLEQQKQQIMAALNSQTAVQFQQYAAQQYPGNYEQQQILIRQLQEQHYQQYMQQLYQVQLAQQQAALQKQQEVVMAGASLPASSKVNTAGASDTLSVNGQAKTHTENSEKVLEPEAAEEALENGPKDSLPVIAAPSMWTRPQIKDFKEKIRQDADSVITVRRGEVVTVRVPTHEEGSYLFWEFATDSYDIGFGVYFEWTDSPNAAVSVHVSESSDEEEEEEENVTCEEKAKKNANKPLLDEIVPVYRRDCHEEVYAGSHQYPGRGVYLLKFDNSYSLWRSKSVYYRVYYTR

SEQ ID NO: 143 - DNA, 1842 bp, D. melanogaster
actttaggtgcgcagctggctggtcacactggcagcagacgtcacaataaattctattattttgttttgttttgtgcgaaaagtttgcaggccagcaatcgtggactagcccacaaacaacggcagcatggcgagcgggacaagcggcacaagcagcagtgaaacggcccagtcgacggacatggcaccatcatcggccgagaagtggggcttccctttgattgagctctaccgcctggcattcacgttctacaagcggaactccggcaaggccatccacttgtcgtacgaggacaacctcaagctgatcgccttcaagcagcaggcggcgctgggccccttcaacacgagtcgcgccccggcgctgggcgtcctggacgtgatcggacgcgaccggcagcagcactggcagctgctcggtgagataacgcgcgagcaggcgatggaggggttcgtcgacctgctggacacaatgtgcagcgcctttcggccgtacattgaggccgtgcggcaggaccgcgacgagacgctgcgcaaggagctgcgcctcatggaggagaagaaggaggcacgcgagcggcaggagaacgcccagcaggagctgctcgaggagggttacaaggaggagctgcagcgccgccagctgcaggacgcactcaacaagcagacttaccagcagtttaagctgtacgctgagaagcagttccccggaaaccccgaacagcaggccgtgcttatccaccagctgcagcgggagcactatcaccagtacatgcaacagcttcatctgcagaaccagaaccagaatcagaatcagaacacggaggacaagggacaccaggaagcagaacacgtccctggcaataacaacaacagctcaaccgacttgcccaacgccatggagggactgaagctcggtgaggtcgagacacagcagcaccagcagctggccgagcagcaagacgggcatgtagagcagacgttggagggcgttgggcaggaacagcacggcgaggaagagtacgacgactacgttatgatctgcccggccaagatctggactcgtcccgacatcgagcagtttaagactgaggtgtccgccggcgatggggacggcgtcatcaccattggacacggcgacacggtgacggtccgtgtgcccaccaacatgaacggcaagtgcatcttctgggagttcgccacggacagctacgacatcggcttcggcatctacttcgagtgggccaagcccgtgaccaacgaggtgaccgtgcatgtgagcgactccgacgaggacgaggattgcgtggacgaggactacctatccaccaccgaggacctggagtccggctcgctgtcgcaggagcgcggcgcagttaacaacccgactgctgcccccaaggcacccatttccattatcgtaccgatctatcgacgggagtgctacaacgaggtctacgtgggctcccactcctatccgggcgagggcgtctatttgctgaagttcgacaacagctacagcatctggcgcaataagacgctgtactaccgcgtctactacgagcgttaagaggccaaaatgtatttacttaccactaacttagatgaatcctatctccgtacgaacggctaatgtgcaagtccggaataatctgtatgtgcgtgtgcgtgaggttttggttttacgtgtgcgttgctgcaattccgggaaacaagcggcgggaaaagaccatgccgatttcagactaaaccaatattatttattatttagttgtaaccgacgaagacaagtccaaaattcaatttgaaactaataaacaatataaagacaactgtaaataa

SEQ ID NO: 144 - protein, 480 aa, D. melanogaster
MASGTSGTSSSETAQSTDMAPSSAEKWGFPLIELYRLAFTFYKRNSGKAIHLSYEDNLKLIAFKQQAALGPFNTSRAPALGVLDVIGRDRQQHWQLLGEITREQAMEGFVDLLDTMCSAFRPYIEAVRQDRDETLRKELRLMEEKKEARERQENAQQELLEEGYKEELQRRQLQDALNKQTYQQFKLYAEKQFPGNPEQQAVLIHQLQREHYHQYMQQLHLQNQNQNQNQNTEDKGHQEAEHVPGNNNNSSTDLPNAMEGLKLGEVETQQHQQLAEQQDGHVEQTLEGVGQEQHGEEEYDDYVMICPAKIWTRPDIEQFKTEVSAGDGDGVITIGHGDTVTVRVPTNMNGKCIFWEFATDSYDIGFGIYFEWAKPVTNEVTVHVSDSDEDEDCVDEDYLSTTEDLESGSLSQERGAVNNPTAAPKAPISIIVPIYRRECYNEVYVGSHSYPGEGVYLLKFDNSYSIWRNKTLYYRVYYER

SEQ ID NO: 145 - DNA, 1308 bp, C. elegans
atggccgacgagaaccacgtccgtctaagcgaggttcaactgattcagtcggaattcggacattctctcgaggaatgctacaagcttgccgttcaatattataaaaaagaacacgtcgggaaacaggagcctgttggatatgaagatcgcattaaattgctctcgttgagcaaacaagtccaacatggagagatttccgatgaatttgacaacgctggatggctagatatcactggaaacgatgtgaacaaagcttggcgtgagctgggctcgttgtcccgcgacgaagcgatggcctcgttcgtcttcctcgtcgatcgtgtgtgtccgccgttcaagggattcattgccgataagaaggctataaaagacgcggaactgaaagaattcgcgccacaggtcactgaacaaagcgcccagccaccgtctcttcaacaagttgatcagagacttttcgaggatcagcggaagcaaatccaagaagcactcaacgcacagaccttccatcaattctcagcctacgctcaggagcaattcccaggacaaccggaacaacagacgacacttattcgacagctccaagagcaacactatcaacaatacatgtctcaggtctatgctcaacaatcgactacgccgaacggtgcagaaatgaatccagaggagtcgcatcaacatcagattcgtcgtgatgatgattcggatgtttcggatgatgaagctggggaggatttgccttccaaccctgcaatttcgcccgcctcactctggaatcggcaggatattaatgagttcaaggcgaatatcaagaaagacggacacgagggaattattaaagttggacacggcgagacggtgacggtccgggtcccaactcacgaaaatggttcatgtctattctgggaattcgcgacggatcactacgatattggtttcggtgtctactttgaatggaccgtagccgacagtaatcaagtttcggtgcacgttagtgaatccgatgatgaggaggattatgatgaagcacttgaagcggagcaggcggaagctggaggtggtggtggaggagccgctggacagggtggaccaggagatgtggaagctggagccatgcagacaagacgtgttgatccgaacaagccgcggcaggatgaaataattccggtgtatcgtcgagattgtcacgaagaagtgtatgcgggaagtcatcgttatccgggtcgcggaatttatctgctcaaattcgacaattcgtattctttgtggcgttcgaagacgctctactatcgtgtctactacagtaaataa

SEQ ID NO: 146 - protein, 435 aa, C. elegans
MADENHVRLSEVQLIQSEFGHSLEECYKLAVQYYKKEHVGKQEPVGYEDRIKLLSLSKQVQHGEISDEFDNAGWLDITGNDVNKAWRELGSLSRDEAMASFVFLVDRVCPPFKGFIADKKAIKDAELKEFAPQVTEQSAQPPSLQQVDQRLFEDQRKQIQEALNAQTFHQFSAYAQEQFPGQPEQQTTLIRQLQEQHYQQYMSQVYAQQSTTPNGAEMNPEESHQHQIRRDDDSDVSDDEAGEDLPSNPAISPASLWNRQDINEFKANIKKDGHEGIIKVGHGETVTVRVPTHENGSCLFWEFATDHYDIGFGVYFEWTVADSNQVSVHVSESDDEEDYDEALEAEQAEAGGGGGGAAGQGGPGDVEAGAMQTRRVDPNKPRQDEIIPVYRRDCHEEVYAGSHRYPGRGIYLLKFDNSYSLWRSKTLYYRVYYSK

SEQ ID NO: 147 - DNA, 3373 bp, Rat
cgggcgggcgtggagctgtctgcacgaaaggactaagattccagatggcaaattctatgaatggcagaaatcctggtggtcgaggaggaaacccccgcaaaggtcgaattttggggattattgatgccattcaggatgcagttggacccccaaagcaagctgcagctgaccgcaggacagtggagaagacttggaaactcatggacaaagtggtaagactgtgccaaaatccgaaacttcagttgaaaaacagcccaccatatatcctcgacattttacctgatacgtatcagcatttgcggcttatattgagtaagtatgacgacaaccagaagctggctcaactgagcgagaatgagtactttaaaatctacatcgacagtctcatgaagaagtcaaagcgagcgatccggctcttcaaagaaggcaaggagaggatgtacgaggagcagtcgcaggacagacggaatctcacaaagctgtcccttatcttcagtcacatgctggcagaaatcaaggcgatctttcccaatggccagttccagggagataacttccggatcaccaaagcagatgctgccgaattctggaggaagttttttggagacaaaactatcgtaccatggaaagtcttcagacagtgcctgcatgaggtccatcagatcagctctggcctggaggccatggctctgaagtcaaccattgacttaacttgtaatgattacatctccgtgtttgaatttgatatttttaccaggctatttcagccctggggctctattttacggaattggaacttcttagctgtgacacacccggggtacatggcatttctcacatatgatgaagttaaagctcgactacagaaatacagcaccaagcctggaagctacattttccggttaagctgcactcggctgggacaatgggccattggctatgtgactggggacggcaatatcctacagaccatacctcataacaagcccctgttccaagccctgattgatggtagcagggaaggcttttacctttatccagatggacgaagctataaccctgatttaaccggattatgtgaacctacacctcatgatcatataaaagttacacaggagcaatatgaactgtattgtgaaatgggctccacttttcagctgtgcaagatctgtgcagagaatgacaaagatgtcaagatcgagccttgtgggcatctcatgtgcacttcgtgccttaccgcgtggcaggagtctgatggccaaggctgccccttctgtcgctgtgagataaaaggaaccgaacctatcatcgtggatccctttgaccccagagacgaaggctccaggtgctgcagcatcatcgaccctttcagcatccccatgctcgacttggatgatgacgatgatcgagaggagtctctgatgatgaaccggctggcgagtgttcgcaagtgcacagacaggcagaactcgccagtcacatcgccaggatcctcaccccttgcccagagaagaaagcctcagccagaccctctccagatcccccacctcagcctgccaccagtgcctccccgcctggacctcattcagaaaggcatcgtgcgctctccctgtggcagccccacgggctccccgaagtcttctccatgcatggttagaaaacaagacaaaccactcccagcaccccctcctcccttgcgagatcctccgcctccaccagagcggcctccgccaatcccgcctgacagtagactgagcagacacttccaccacggagagagtgtgccttccagggaccagccaatgcctcttgaagcctggtgccctcgggatgccttcgggactaatcaggtgatgggatgtcgcatcctaggggatggctctccaaagcctggcgtcacagcaaactccaacttaaatggacgtcacagtcgaatgggctctgaccaggttcttatgaggaaacacagacgccacgatttgccttcagaaggcgccaaggtcttttccaatggacaccttgcccctgaagaatacgacgttcctcctcggctttcccctcctcctccagtcactgcccttctccctagcataaagtgtactggtccaatagcaaattgtctctccgagaaaacaagagacacagtagaagaagatgatgatgaatacaagattccttcatcccatcctgtttccctgaattcacaaccatctcattgtcataatgtcaaacctcctgttcggtcttgtgataatggtcactgtatactgaatggaactcatggtacgccttcagagatgaagaaatcaaacatcccagatttaggcatctatttgaagggtgaagatgcttttgatgccctccccccatcccttcctcctcccccacctcctgcaagacatagtctcatcgagcattcaaaacctccaggctccagtagccggccttcctcaggacaggaccttttccttcttccttcagatcccttttttgacccagcaagtggccaagttccattgcctccggccaggagagcaccaggagatggtgtcaaatccaacagagcctcccaggactatgaccagctcccttcatcttccgatggttcgcaagcaccagctagaccccccaaaccacgaccccgaaggactgcaccagaaattcatcacagaaagccccatgggcccgaggcggcactggaaaatgtggatgcgaaaattgcaaaactcatgggagaggggtatgcctttgaagaggtgaagagagccttagagatcgcccagaataacctggaagtggccaggagcatacttcgagaattcgccttccctcctcccgtctcgccacgtctcaatctatagcagcccagactgcaaacaccaaagggtaaaacagttaacaaatattccaggagtatgggacagaaggactgagagggaatgcaggagccatggtgtcttttcatgtggcgtctccagaaggcagccttgagtccagcttctctggtaccacagctccctgaggatgcccacgctgcagcttctgtgtttgtgctagccatacttttaaatcagggttgaactgagaaaataatttaaagacgtttactcccccttgaactttgaatctgtgaaatgctttccttgtttacacgttggcagaattgcagtttgtctctgtttttgattcctgtactgtgttcctgacaggcccttggcagagttggtcaggtctgctgtaagtttgtccatgcccaccctgctgcccacattggcagctaaagcatctcttcgtgttgctgtctatccgggccccacctcatgtgtccacgtccagttcatttctctcattcacacagcatgctagtctgagg

SEQ ID NO: 148 - protein, 938 aa, Rat
MANSMNGRNPGGRGGNPRKGRILGIIDAIQDAVGPPKQAAADRRTVEKTWKLMDKVVRLCQNPKLQLKNSPPYILDILPDTYQHLRLILSKYDDNQKLAQLSENEYFKIYIDSLMKKSKRAIRLFKEGKERMYEEQSQDRRNLTKLSLIFSHMLAEIKAIFPNGQFQGDNFRITKADAAEFWRKFFGDKTIVPWKVFRQCLHEVHQISSGLEAMALKSTIDLTCNDYISVFEFDIFTRLFQPWGSILRNWNFLAVTHPGYMAFLTYDEVKARLQKYSTKPGSYIFRLSCTRLGQWAIGYVTGDGNILQTIPHNKPLFQALIDGSREGFYLYPDGRSYNPDLTGLCEPTPHDHIKVTQEQYELYCEMGSTFQLCKICAENDKDVKIEPCGHLMCTSCLTAWQESDGQGCPFCRCEIKGTEPIIVDPFDPRDEGSRCCSIIDPFSIPMLDLDDDDDREESLMMNRLASVRKCTDRQNSPVTSPGSSPLAQRRKPQPDPLQIPHLSLPPVPPRLDLIQKGIVRSPCGSPTGSPKSSPCMVRKQDKPLPAPPPPLRDPPPPPERPPPIPPDSRLSRHFHHGESVPSRDQPMPLEAWCPRDAFGTNQVMGCRILGDGSPKPGVTANSNLNGRHSRMGSDQVLMRKHRRHDLPSEGAKVFSNGHLAPEEYDVPPRLSPPPPVTALLPSIKCTGPIANCLSEKTRDTVEEDDDEYKIPSSHPVSLNSQPSHCHNVKPPVRSCDNGHCILNGTHGTPSEMKKSNIPDLGIYLKGEDAFDALPPSLPPPPPPARHSLIEHSKPPGSSSRPSSGQDLFLLPSDPFFDPASGQVPLPPARRAPGDGVKSNRASQDYDQLPSSSDGSQAPARPPKPRPRRTAPEIHHRKPHGPEAALENVDAKIAKLMGEGYAFEEVKRALEIAQNNLEVARSILREFAFPPPVSPRLNL

SEQ ID NO: 149 - DNA, 3131 bp, Mus musculus
gactccctgggctgcgagcgccggcggtggttgccggagaggcccctccttctcgcccggctccattccctcgctcgcggccgagcgggctcccgaccctccgctggccatggccggcaacgtgaagaagagctcgggcgccggcggcggcggctctgggggctcgggagcgggcggcctgatcgggctcatgaaggacgccttccagccgcaccaccaccaccaccacctcagcccgcaccctccctgcacggtggacaagaagatggtggagaagtgctggaagctcatggacaaggtggtgcggttgtgtcaaaacccaaagctggcgctcaagaacagcccgccttatatcttagacctgctgcctgacacctaccagcacctccgcactgtcttgtcaagatatgaggggaagatggagacgcttggagaaaatgagtatttcagggtgttcatggaaaatttgatgaagaaaactaagcagactatcagcctcttcaaggagggaaaagaaaggatgtatgaggagaattcccagcctaggcgaaacctgaccaaattatccctgatcttcagccacatgctggcagaactgaaaggcatctttccgagcggactcttccaaggagacactttccggattactaaagctgatgctgccgaattttggagaaaagcttttggtgaaaagacgatagtcccgtggaagagctttcgacaggccctgcatgaagtgcatcccatcagttctgggctggacgccatggctctgaagtccactattgatctgacctgcaatgattatatttctgtctttgaatttgatatttttacacggctgtttcagccctggtcctctttgctcagaaattggaacagccttgctgtaactcaccctggttacatggctttcctgacatacgatgaagtgaaagcgcgcctgcagaagttcatccacaaacctggcagttacatctttcggctgagctgtactcgtttgggtcagtgggctattgggtatgttactgccgatgggaacattctgcagacaatcccacacaataaaccgctcttccaagcactgattgatggcttcagggaaggcttctatttgtttcctgatggacgaaatcaaaatcctgacctgacaggtttatgtgaaccaactcctcaagatcatatcaaagtaacccaggaacaatatgaattatactgtgaaatgggctccacatttcaactgtgtaagatatgtgctgagaatgataaggatgtgaagattgagccctgtggacacctcatgtgcacatcctgcctcacgtcgtggcaggaatcagaaggtcagggctgtcctttttgccgatgtgaaatcaaaggtactgagcccatcgtggtggatccgtttgaccccagaggcagtggcagcctattaaggcaaggagcagaaggtgctccttccccaaattacgacgatgatgatgatgaacgagctgatgattctctcttcatgatgaaggagttggcaggtgccaaggtggaaaggccttcctctccattctccatggccccacaagcttcccttcctccagtgccaccaagacttgaccttctacagcagcgagcacctgttcctgccagcacttcagttctggggactgcttccaaggctgcttctggctcccttcataaagacaaaccattgccaatacctcccacacttcgagatcttccaccaccaccccctccagaccggccttactctgttggagcagaaacaaggcctcagagacgccctctgccttgtacaccaggcgattgtccatctagagacaaactgccccctgtcccttctagccgcccaggggactcgtggttgtctcggccaatccctaaagtaccagtagctactccaaaccctggtgatccttggaatgggagagaattgaccaatcggcactcgcttccattctcattgccctcacaaatggaacccagagcagatgtccctaggcttggaagcacatttagtctggatacctctatgactatgaatagcagcccagtagcaggtccagagagtgagcacccaaagatcaagccttcctcgtctgccaacgccatttactctctggctgccaggcctcttcctatgccaaaactgccacctggggagcaaggggaaagtgaagaggacacagaatatatgactcccacatctaggcctgtaggggttcagaagccagagcccaaacggccgttagaggcaacccagagttcacgagcatgtgactgtgaccagcagatcgacagctgtacctatgaagcgatgtataacatccagtcccaagcactgtctgtagcagaaaacagcgcctctggggaagggaatctggccacagctcacacgagtactggccctgaggaatccgaaaacgaggatgatggctatgatgtgcctaagccacccgtgccagctgtactggcccgccggaccctgtctgacatctccaatgccagctcctcctttggctggttgtctttggatggtgaccctacaaacttcaatgagggttcccaagttcctgagcggccccccaaaccattccctcggagaatcaactcagaacgaaaagccagtagctatcaacaaggcggaggtgccactgctaaccctgtggccacagcaccctcaccgcagctctcaagtgagattgaacgcctcatgagtcagggctattcctaccaggacattcagaaagctttggtcattgcccacaacaacattgagatggctaaaaacatcctccgggaatttgtttctatttcttctcctgctcacgtagccacctagcacatctctccctgccacggcttcagaggacccatgagccaggctcttactcaaggaccacctaggaaagcagtggcttcttttgggacgtcacagtaaggtcctgcctttcctgtggggatcgacacatatggttccaagatttcaaagcagtggaatgaaaatggagcagctgatgtgtttcattgttgtattggtcttaagagtgtttttgtagtcctgcagtctccagtaggagagagtgggtttttattaaatggtaacctaccccagaaacagc

SEQ ID NO: 150 - protein, 913 aa, Mus musculus
MAGNVKKSSGAGGGGSGGSGAGGLIGLMKDAFQPHHHHHHLSPHPPCTVDKKMVEKCWKLMDKVVRLCQNPKLALKNSPPYILDLLPDTYQHLRTVLSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLDAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDSLFMMKELAGAKVERPSSPFSMAPQASLPPVPPRLDLLQQRAPVPASTSVLGTASKAASGSLHKDKPLPIPPTLRDLPPPPPPDRPYSVGAETRPQRRPLPCTPGDCPSRDKLPPVPSSRPGDSWLSRPIPKVPVATPNPGDPWNGRELTNRHSLPFSLPSQMEPRADVPRLGSTFSLDTSMTMNSSPVAGPESEHPKIKPSSSANAIYSLAARPLPMPKLPPGEQGESEEDTEYMTPTSRPVGVQKPEPKRPLEATQSSRACDCDQQIDSCTYEAMYNIQSQALSVAENSASGEGNLATAHTSTGPEESENEDDGYDVPKPPVPAVLARRTLSDISNASSSFGWLSLDGDPTNFNEGSQVPERPPKPFPRRINSERKASSYQQGGGATANPVATAPSPQLSSEIERLMSQGYSYQDIQKALVIAHNNIEMAKNILREFVSISSPAHVAT

SEQ ID NO: 151 - DNA, 2661 bp, D. melanogaster
catctcgaaaatattgtgtgggtttaaaaaacgttaacgtcgccgaaacgcgtagccccaaatgcacacgccaggtgcaaggataaagccgtgaggatcgggcacccaatcggatagatcgcgtttggttagcttgtgggggaaaatcgtacttaagtcaccactactactacacacgggcaccaccagcaacaccaacaacaacaacaacgagaacagcaccagcaacaacaacaacagcagcaagaaggagaagagctgagaagaggaagcagaggcagcgcagtcggcagcgcagcggcagagagaaaagatggcgacgagaggcagtggaacccgtgtgcaatcgcagccaaagattttcccatcgctgctttccaagctgcacggcgctatctcggaagcctgcgtctcgcagcgtctgtccaccgacaagaagacgctggagaagacctggaagttgatggacaaggtggtcaaactgtgccagcagccgaagatgaatcttaagaatagtccaccgtttattttggacatcctgccggatacgtaccagcgcctgagattgatctactcaaagaaggaggaccagatgcacctgctccatgccaacgagcacttcaacgtgttcatcaacaacctgatgcgaaagtgcaagcgggccatcaagttgttcaaggagggcaaggagaagatgttcgacgagaactcccactaccgccgcaatctcaccaagctcagcctggtcttctcccacatgctcagcgaactgaaggccatattccccaacggtgtctttgccggggatcaatttcggatcaccaaagcggatgcggctgacttttggaagagcaacttcggtaacagcacattggttccctggaaaatcttccggcaggagcttagcaaagtacatcccataatctccggcctggaggccatggccctaaagaccactatcgatcttacctgcaacgacttcatttccaacttcgagttcgacgtcttcacacgcctcttccagccttgggtgacactgctacgcaactggcagattctggccgtcacacatccgggctacgtggcgtttctcacatacgacgaggtgaaggctcgcctacagcgctacatcctcaaggcgggcagctacgttttccggctctcctgcacgcgattgggccaatgggccatcggctacgtaactgccgagggagagattctgcagacaatccctcagaacaagtcgctgtgccaggcgctgctcgatggccatcgagagggcttctacttgtacccagatggccaagcgtacaatccggatctgtcgtctgccgttcaaagtcccacagaggaccacataaccgttacccaagagcaatacgaactatactgtgaaatgggcagcacctttcagctgtgcaaaatttgtgcggagaacgacaaagatatccgcatcgagccctgtggccacttgttgtgcactccctgccttacctcctggcaagtggattccgagggacagggctgccccttctgtcgggccgaaatcaagggcaccgaacaaatcgttgtggacgctttcgatccgcgcaagcaacacaaccggaacgtcaccaatgggcgacagcagcagcaggaagaagacgacactgaggtatagttttgttcacagcctgatcagcctgatccgcctgctccgctgccgcctgtgctgctatttatatacatattactcttatgattacctttggttcgtttatacagttatatatgcctatatatacattatatattttagattttacaactgctattgtttatataagtttaatgtttagcctgcagttcgcagtggcagtttcgagtttaattttgtttgtttagctgtaacatatttaaattattagccaaactcatgcaactaacatccacagacccacgcacacacgcccaatcacaagcacaagtacaaccataaccattgtccatccatcgagcacatgcataacgtagttaaagttctttgaccggaagtcgctcatcaaccatcgtttgctatcgcttcctctgttttctctccgccggtttggtttggtttggtttgtgtgcgttcgtttagttgttcgttcttccactctcacgctctctctatctattgatcacgttcgcctctgtttatgaatcatattttaatcgattcgattcgccctcgattgcacttttgtacataggcactatggaatttataattggtaaccttgttcttgtattattcgggtgaattttctcctttcacatccagcttgattatccccttgattatgtatgcccgccagtaatttttgtatctatcccctactctagaatcattctcttaatcattgtactccgttatgtgtttatttcattttagtttattgtttaatacttccaaagatacatttagtttgtagtagcgtgcgtttacttcccccccatatcaattcaattttatttgtaagcagccaaygcgctgccctaagactgtaatttattattaacamaaaaaaraaaatcgaaaaagtttaagaaatcaggctaaacataggaggcctcgaatcgatcgataatttagttagattgyatgtaaattaattattgatttcctgtgtcacaaggcca

SEQ ID NO: 152 - protein, 448 aa, D. melanogaster
MATRGSGTRVQSQPKIFPSLLSKLHGAISEACVSQRLSTDKKTLEKTWKLMDKVVKLCQQPKMNLKNSPPFILDILPDTYQRLRLIYSKKEDQMHLLHANEHFNVFINNLMRKCKRAIKLFKEGKEKMFDENSHYRRNLTKLSLVFSHMLSELKAIFPNGVFAGDQFRITKADAADFWKSNFGNSTLVPWKIFRQELSKVHPIISGLEAMALKTTIDLTCNDFISNFEFDVFTRLFQPWVTLLRNWQILAVTHPGYVAFLTYDEVKARLQRYILKAGSYVFRLSCTRLGQWAIGYVTAEGEILQTIPQNKSLCQALLDGHREGFYLYPDGQAYNPDLSSAVQSPTEDHITVTQEQYELYCEMGSTFQLCKICAENDKDIRIEPCGHLLCTPCLTSWQVDSEGQGCPFCRAEIKGTEQIVVDAFDPRKQHNRNVTNGRQQQQEEDDTEV

SEQ ID NO: 153 - DNA, 2274 bp, C. elegans
ctatgatcattacatcctaattaattgccactggacttcacatcatatcaccgtttcaccgggaatgggttcaataaacacaatttttcaccggatacatcggtttgtcaatggcacaggcaataatgcgcgatttgttcccagcacaaacaactcgacggaagcgttgacactcagtccgagagctgttcccagcacagtttcactattcgaaatcccatcagcttcggagatgcccggtttctgcagtgaagaggatcgtcgatttttgctcaaagcatgcaagtttatggatcaagtagtgaagagttgtcatagcccaagactgaatttgaaaaattcgccgcctttcattttggacattctacctgatacttatacgcatttaatgctgatattcacacaaaacaatgacatactccaagacaacgactacttgaaaatctttctggagagtatgatcaacaagtgcaaagagatcatcaaactgttcaagacgtcagctatctacaatgaccagtctgaagaacgacggaagcttacgaaaatgtcactaacattttcacatatgcttttcgagattaaagcattatttccggaaggtatctatattgaagaccggtttcggatgacaaagaaggaagccgaaagcttttggagtcatcattttacaaaaaaaaacattgtaccctggtcaacattttttactgcattagaaaagcaccatggatcaacgataggaaaaatggaagcagccgaattaaaagctacgatagacttgagcggagatgattttatttcgaattttgagtttgatgtgtttacaaggttattctaccctttcaaaacactgatcaaaaattggcaaacactcaccaccgcccatcccggatactgtgcatttctcacatacgatgaggtcaaaaaacggttagaaaaattaacgaaaaaacctggaagctacatcttccggttatcatgcacacgtcctggacaatgggcaataggatacgtagctccggatggaaagatttatcagacaataccacagaataaaagtttgattcaagcactacatgaaggccataaagaaggattttatatttacccgaacggtagagatcaagatattaacttatccaaattgatggatgtgccacaagcggacagagtgcaagtgaccagtgaacaatacgagttgtattgtgagatgggcacaacattcgagttgtgcaaaatttgtgacgataacgagaagaacatcaaaattgagccatgtggacatttgctctgcgcaaaatgtttggctaactggcaggattcggatggtggtggcaacacatgtccattctgccgctacgaaatcaaaggaacaaatcgtgtgattattgacaggttcaagcccactccggtagaaattgaaaaagcgaaaaatgtagctgctgcggagaagaagctgatctcattagttcccgacgtgcctcccagaacgtatgtgtcccaatgttctcaaagtttgctgcatgacgcgtcaaactcaattccgtcggtcgacgagttgccgttggtgccgccaccgttgccaccgaaagcattgggtaccctggacactttgaattcgtcacaaacatcctcttcatacgtgaacatcaaagagctggaaaatgttgaaacaagcggagaagcattggcacaagtggtaaaccggcaacgggcgccttcaatccaagctccaccactaccgccaaggttatcagcgagcgagcaccaaccacaccacccatacacaaatacgaacagtgagcgggagtagacttgtgtaaatgttcatcttaccgctttatactgcaattttcattcccccacttatcatagaactattcttccacaacaacatattgccgtgactagaactggtaacactacatcattctttgttaaaacgttattatatctctatttctttttcgcctactcctttccgtttttttttcaaattttgtcaattttcctacagcgttctgactcctattggtaagcaatcatgtcatatcttgttaaattttcatgttaatttcttactctcgctgtcccagattttacggagttttcaggaaacgtttgattttgttctattctacaatttccatcgcccccaacctgtcgtgtattttctatgtgtcactctgaagaaaacaagtttagactttttaaaaatcgttttattactctaaaacttaaaagctgaaatgtcagctatagtaaaaatacata

SEQ ID NO: 154 - protein, 582 aa, C. elegans
MGSINTIFHRIHRFVNGTGNNARFVPSTNNSTEALTLSPRAVPSTVSLFEIPSASEMPGFCSEEDRRFLLKACKFMDQVVKSCHSPRLNLKNSPPFILDILPDTYTHLMLIFTQNNDILQDNDYLKIFLESMINKCKEIIKLFKTSAIYNDQSEERRKLTKMSLTFSHMLFEIKALFPEGIYIEDRFRMTKKEAESFWSHHFTKKNIVPWSTFFTALEKHHGSTIGKMEAAELKATIDLSGDDFISNFEFDVFTRLFYPFKTLIKNWQTLTTAHPGYCAFLTYDEVKKRLEKLTKKPGSYIFRLSCTRPGQWAIGYVAPDGKIYQTIPQNKSLIQALHEGHKEGFYIYPNGRDQDINLSKLMDVPQADRVQVTSEQYELYCEMGTTFELCKICDDNEKNIKIEPCGHLLCAKCLANWQDSDGGGNTCPFCRYEIKGTNRVIIDRFKPTPVEIEKAKNVAAAEKKLISLVPDVPPRTYVSQCSQSLLHDASNSIPSVDELPLVPPPLPPKALGTLDTLNSSQTSSSYVNIKELENVETSGEALAQVVNRQRAPSIQAPPLPPRLSASEHQPHHPYTNTNSERE

SEQ ID NO: 155 - DNA, 3463 bp, Rat
cggtgaggggagccgggcgggtggcagagcagagcaggaggaagcaacggccacagacgcatcgggaccgcgagtcgtagtcgggggatccgcggggcggaggcgggcaccatgggcaaccgcgggatggaagagctcatcccgctggttaacaagctgcaggacgccttcagctccatcggtcagagctgccacctggacctgccgcagatcgccgtggtgggcggccagagcgccggcaagagttcggtgctcgagaacttcgtgggccgggacttccttccacgaggatcaggaattgtcacccggaggcctctcattctgcagctcatcttttccaaaacagaatatgcggagtttttgcactgcaagtccaaaaagtttacagactttgatgaagtccggcaggagatcgaagcagagactgaccgggtcacaggcaccaacaaaggcatctccccggtgcccatcaaccttcgggtctattcaccacacgtgttgaacttgaccctcatcgacctcccaggcatcactaaggtgccagttggggaccagccaccagacatcgagtaccagatcaaggacatgatcctgcagtttatcagccgggagagcagcctcattcttgccgtcacacctgccaacatggacttggccaactcggatgccctcaagctggccaaggaggtggacccccaaggcttgcggaccattggtgtcatcaccaagctagacctgatggatgaaggcacagatgccagggatgtcctggagaacaagctcctgcccttgagaagaggctacatcggcgtggttaaccgcagccagaaagacatcgagggcagaaaggacatccgggcagctctggcagctgagaggaaattcttcctctcccacccagcctaccggcacatggctgaccgcatgggtaccccacacttgcagaaaacactgaaccagcaactgaccaaccacatccgggagtcactgccaaccctgcgcagcaagctgcagagccaactgctgtccctggagaaggaagtggaagagtacaagaatttccggcctgacgaccccacacgcaagaccaaagccctgctgcagatggtccagcagtttggagtggactttgagaagcgaatcgaaggctctggagatcaagtcgacaccctggagctgtctgggggcgcccgcatcaatcgcatcttccacgagcgctttccctttgagctggtgaagatggagtttgacgagaaagacctccgaagagagatcagctacgccattaagaacatccacggagtcagaactgggctcttcacgccggatttggcgttcgaggccattgtgaaaaagcaggtggtcaagctgaaagagccctgtctgaaatgcgtggacctggttatccaggagctaatcagtacagttaggcaatgcaccagcaagctcagttcctacccccgccttcgagaggagaccgaacgcattgtcaccacctacatcagggagcgagaagggagaaccaaggaccagatccttcttctgattgacattgagcagtcctacatcaacacaaaccatgaagacttcattggatttgccaatgcccagcagaggagcacgcagctgaacaagaagagggccatacctaaccagggggagatcttggtgatccgcaggggctggttgaccatcaacaacatcagcttgatgaaaggcggttccaaggagtactggttcgtgctgactgctgagtcgttgtcttggtacaaggatgaagaggaaaaagaaaagaagtacatgctgccactagacaacctcaaaatacgggatgtggagaagggcttcatgtccaacaagcatgtgtttgccatcttcaacacagagcagaggaacgtctacaaggacctgcgacagatcgaactggcttgtgactcccaggaagatgtggacagctggaaggcttcgttcctgcgagctggggtctacccagagaaggaccaggcagagaacgaggatggagcacaagagaacaccttctccatggacccgcagctggagcggcaggtggagaccatccgcaacttggtggactcctatgtggccatcatcaacaagtccatccgggacctcatgccaaagaccatcatgcacctcatgatcaacaacacaaaggccttcatccaccatgagctgctagcctacctgtactcatcagcagaccagagcagcctcatggaggagtcagctgagcaggctcagcggcgggacgatatgctgcgcatgtaccatgcactcaaggaagcgctcaacatcattggggacatcagcaccagcaccgtgtccacgcctgtgcccccacctgtcgatgatacttggctccagaacaccagcagccacagccccactccacagcgcagacctgtgtccagtgtgcacccgccgggccgcccacccgcagtgcggggccccacaccggggcctcccctcattcctatgcctgtgggggccacatcctccttctcggcaccccccatcccatcccggcctggaccacagaatgtgtttgccaacaatgaccccttctctgcgccacctcagataccgtctcggccagcacggattccccctgggatccctccaggagtgcccagcagaagagcgcccgctgcgcccagccggcccaccattatccgcccagccgagccgtccctgctcgactaggctgcaggggacagtgttctgggggggtcctcgtgtacctacagtggtctgggccctgctactgccccccatgctgggaccaggctccccgtgggcaagcctgccctctcctccctagccctagtgcacatgataaggagggagagggtgggaaggaggcccaaacaaacagaacttgggggctttgcactttggggcgagggggagctctggttagcagaacagaaactgcacccttgggaccatctggaaagaaaggtctaggctagtggtggggaactctacatgggctcctccatactgtaagcctttgctctttctgaggtacagaactgtccacctttccctaaggactcctcaggagttagccaggcccaggaggtccagtgctggccacccacctggctcaagttgtatatatatatatagcttctttggccacattaaccacacagcctgagcccagcccagtctccacacccagaggtgcctttgtgccttccaggcctggagtgcctcagctctggcctgcctctctcctcagcccctgttgtggtttgggctgtaagaactctggagacatcaggacctcttggcttccaaccagatgtctcctacagggtgggccctggtgcctctgtacagattctgctcccaacgcttggattgaccactgttaagtgcctgcctctgtatatcctattaataaactaaaataaagggaagccctctcgtggct

SEQ ID NO: 156 - protein, 870 aa, Rat
MGNRGMEELIPLVNKLQDAFSSIGQSCHLDLPQIAVVGGQSAGKSSVLENFVGRDFLPRGSGIVTRRPLILQLIFSKTEYAEFLHCKSKKFTDFDEVRQEIEAETDRVTGTNKGISPVPINLRVYSPHVLNLTLIDLPGITKVPVGDQPPDIEYQIKDMILQFISRESSLILAVTPANMDLANSDALKLAKEVDPQGLRTIGVITKLDLMDEGTDARDVLENKLLPLRRGYIGVVNRSQKDIEGRKDIRAALAAERKFFLSHPAYRHMADRMGTPHLQKTLNQQLTNHIRESLPTLRSKLQSQLLSLEKEVEEYKNFRPDDPTRKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELISTVRQCTSKLSSYPRLREETERIVTTYIREREGRTKDQILLLIDIEQSYINTNHEDFIGFANAQQRSTQLNKKRAIPNQGEILVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESAEQAQRRDDMLRMYHALKEALNIIGDISTSTVSTPVPPPVDDTWLQNTSSHSPTPQRRPVSSVHPPGRPPAVRGPTPGPPLIPMPVGATSSFSAPPIPSRPGPQNVFANNDPFSAPPQIPSRPARIPPGIPPGVPSRRAPAAPSRPTIIRPAEPSLLD

SEQ ID NO: 157 - DNA, 3458 bp, Mus musculus
gaattcaagcgcgtgccgcgacaggggagaccgggcgggccgcagagcagagcaggaggaagcagcggccacagacacatcggaaccgagagtcttagtcgggggatccgcgcggcgaggcggacaccatgggcaaccgcgggatggaagagctcatcccgctggttaacaagctgcaggacgccttcagctccatcggtcagagctgccacctggacctgccgcagatcgccgtggtgggcggccagagcgccggcaaaagttcggtgctcgagaacttcgtgggccgggacttccttccacgaggatcaggaattgtcacccggaggcctctcattctgcagctcatcttttccaaaacagaatatgcggaatttttgcactgcaagtccaaaaaatttacagactttgatgaagtccggcaggagatcgaagcagagactgaccgggtcacaggcaccaacaaaggcatctcccccgtgcccatcaaccttcgggtctactcaccacacgtgttgaacttgaccctcatcgacctcccaggcatcactaaggtgccggtgggggaccagccgccagacatcgagtaccagatcaaggacatgatcctgcagttcatcagccgcgagagcagcctcattcttgccgtcacacctgccaacatggacttggccaactcagacgccctcaagctggccaaggaggtggacccccaaggcctacggaccatcggtgtcatcaccaagctagacttgatggatgaaggcacagacgccagggatgtcctggaaaacaagctactgcccttgagaagaggctatatcggcgtggttaaccgaagccagaaagacatcgagggcaaaaaggacatccgggctgctctggcagccgagaggaaattcttcctctcccacccagcctaccggcacatggctgaccgcatgggcaccccacacttgcagaaaaccctgaaccagcaactgaccaaccacatccgagagtcactgccgacccttcacggcaagctgcagagccaactgctgtccctggagaaggaagtggaagagtacaagaatttccggcctgatgaccccacgcgcaagaccaaagccctgctgcagatggttcagcagtttggagtggactttgagaagcgaattgaaggctcgggagatcaagtagacacactagagttgtctggtggagcccgcatcaatcgtatctttcatgagcgctttccctttgaactggtaaagatggagtttgatgagaaagatctacgaagagagatcagctatgctattaagaacatccacggagtcagaactgggctcttcacgccggatttggcgttcgaggccattgtgaaaaagcaggtggtcaagctgaaagagccctgtctgaaatgcgtggacctggttatccaggagctaatcagtacagttaggcagtgcaccagcaagctgagttcctacccccggctgcgagaggagaccgagcgaattgtcaccacctacatcagggagcgagaagggagaaccaaggaccagattcttcttctgattgacattgagcagtcgtacatcaacacaaaccatgaagacttcattggatttgccaatgcccagcagaggagcacgcagctgaacaagaagagggccatacccaatcaggtgatccgcaggggctggttgaccatcaacaatatcagcttgatgaagggtggctccaaggagtactggttcgtgctgacagctgagtcattgtcttggtacaaggatgaggaggagaaagaaaagaagtacatgctgccactggacaacctcaagatccgggacgtggaaaagggcttcatgtccaacaagcacgtgtttgccatcttcaacacagagcagaggaatgtctacaaggaccttcgacagattgaactggcttgtgactcccaggaagatgtggacagctggaaggcttcattcctgcgcgctggggtctaccccgagaaggaccaggcagagaatgaggatggagcacaagagaacaccttctccatggacccgcagctggagcgacaggtggagaccatccgtaacctggtggactcctacgtggccatcatcaacaagtccatccgtgaccttatgccaaagaccatcatgcacctcatgatcaacaacacaaaggccttcatccaccatgagctgctggcttacctgtattcatcagcagaccagagcagcctcatggaagagtcagccgagcaggctcagcggcgggacgacatgctgcgcatgtaccacgcactgaaggaggccctcaacattattggggacatcagcaccagcaccgtgtccacacccgtgcccccaccggtcgatgacacgtggctccaaaacaccagcggccacagccccactccacagcgccgacctgtgtccagtgtgcacccaccaggccggccacctgcagtgcggggtcctacaccggggcctcccctgattcctatgcctgtgggggccacatcctccttctcagcacctcccatcccatcccggcctggaccacagagtgtgtttgccaacaacgaccccttctctgcgccacctcagataccatctcggccagcacggattccccctggtatccctccaggagtgcccagcagaagagcgcccgctgcgcccagccggcccaccattatccgcccagccgagccgtccctgctcgactaggctgcagggggcaatgttctgggggggtcctcgtgcacccacagtgtaggacagctttggtggtctgggccctgctacttgccctccatgctgggaccaggctccccgtgcaagccctgccctttccctcctcgccctagtggacatgacaatgaagggtgaggaggcccacagcacagcacatgggggctttgcactttgggatgaggaggagctctggctagcagaacaggaactgcatccttgggaccatcaggaaaaaaaggtccaggctagtgtggggggactcttctggagctcctccagagcctttgttctttctggggtccaggaactgcccacctttcctaaggactcctcaggagtgagccaggcccagcaggtccagtgctgacccacctggctcaagttgtatatatagagctcctttggccatattaaccacacaagctgagcccagcccaggctccgacccagaggtgcctttgtgccttcctcagcctggagggcctcagctctggccaccctcactctcctcagccccggttgtggtttgggctgtatgaactggggagacatcagaggcctcttggctcccaaccagatgtcccacagggtgggccctgggtgcctctgcagcctcggctcccaccgcctggattgaccactgttaagtgcctgcctctgtatatcctattaataaactaaaataaagggaaaaaaaagctt

SEQ ID NO: 158 - protein, 866 aa, Mus musculus
MGNRGMEELIPLVNKLQDAFSSIGQSCHLDLPQIAVVGGQSAGKSSVLENFVGRDFLPRGSGIVTRRPLILQLIFSKTEYAEFLHCKSKKFTDFDEVRQEIEAETDRVTGTNKGISPVPINLRVYSPHVLNLTLIDLPGITKVPVGDQPPDIEYQIKDMILQFISRESSLILAVTPANMDLANSDALKLAKEVDPQGLRTIGVITKLDLMDEGTDARDVLENKLLPLRRGYIGVVNRSQKDIEGKKDIRAALAAERKFFLSHPAYRHMADRMGTPHLQKTLNQQLTNHIRESLPTLHGKLQSQLLSLEKEVEEYKNFRPDDPTRKTKALLQMVQQFGVDFEKRIEGSGDQVDTLELSGGARINRIFHERFPFELVKMEFDEKDLRREISYAIKNIHGVRTGLFTPDLAFEAIVKKQVVKLKEPCLKCVDLVIQELISTVRQCTSKLSSYPRLREETERIVTTYIREREGRTKDQILLLIDIEQSYINTNHEDFIGFANAQQRSTQLNKKRAIPNQVIRRGWLTINNISLMKGGSKEYWFVLTAESLSWYKDEEEKEKKYMLPLDNLKIRDVEKGFMSNKHVFAIFNTEQRNVYKDLRQIELACDSQEDVDSWKASFLRAGVYPEKDQAENEDGAQENTFSMDPQLERQVETIRNLVDSYVAIINKSIRDLMPKTIMHLMINNTKAFIHHELLAYLYSSADQSSLMEESAEQAQRRDDMLRMYHALKEALNIIGDISTSTVSTPVPPPVDDTWLQNTSGHSPTPQRRPVSSVHPPGRPPAVRGPTPGPPLIPMPVGATSSFSAPPIPSRPGPQSVFANNDPFSAPPQIPSRPARIPPGIPPGVPSRRAPAAPSRPTIIRPAEPSLLD

SEQ ID NO: 159 - DNA, 3013 bp, D. melanogaster
gatctgatatctaatctaatctaattgtgagggaaatcggacctcgccgcaatggatagtttaattacaattgttaacaagctgcaagatgcgttcacatcgctcggcgtccacatgcaactggatctgccgcagatcgcggtggtcggtggccagtcagctggcaagagttccgttttggagaactttgtgggcaaagatttcttgccgcgcggctctggtatcgtaactcggagaccactaatcttgcaactgatcaatggagttaccgaatatggcgagttccttcacattaaaggcaagaagttttccagcttcgatgagatcagaaaagaaatcgaagatgaaacggatcgtgtgaccggtagtaacaaaggcatttcaaacattccaattaatttgcgcgtctactcgccgcacgtactcaatttaaccctgatcgatttgccaggcctgacaaaggtggccattggcgatcaaccggtggatattgagcagcaaattaagcagatgatattccaatttattcgcaaagagacttgtttaattttggctgtgaccccggccaatacggatctggccaattcggatgccctcaagctggccaaggaggtggatccacagggtgtgcgaaccattggcgttataaccaagctggatctgatggacgagggcaccgatgcacgcgacattctggagaataagctgcttccgctgcgacgtggctatattggcgtggtgaatagatcccaaaaggatatcgaggggcgcaaggatatccaccaggcattggccgccgaacgcaagttcttcttgagccatccctcctatcggcacatggccgaccgtctcggcaccccctacttgcagcgcgtgctcaaccaacaactaaccaatcacatcagggacacgttacctggcctgcgggataagttgcagaagcagatgctcactttggagaaggaggtggaggagtttaagcactttcagccaggcgatgctagcatcaaaacaaaagctatgctccaaatgattcagcagctgcaatcagactttgagcgaactatcgagggcagcggctcagctttggtgaacacaaacgagctctcaggtggcgccaagatcaatcgcattttccatgaacgtctgcgctttgagatcgtaaagatggcttgcgatgaaaaggaactgcgacgagagatctcatttgcaattcgaaatatccatggtattcgcgttggcctcttcacacccgacatggcgttcgaggcgattgtcaagcggcagatagcgctgctcaaggagccggttatcaagtgtgtcgatctagtcgtacaggaactttctgtggtcgtgcgcatgtgcacagctaaaatgagtcgatacccacgtctgcgtgaggagaccgaacgtattatcacaacacatgtgcgccaacgcgagcatagctgcaaggaacagatcctgctgcttattgactttgagctggcctatatgaacaccaatcatgaggacttcattggcttcgccaacgctcaaaacaaatcagagaatgccaacaaaactggcactcgtcaactgggcaaccaagtcattcgcaagggtcacatggtcatccagaaccttggaatcatgaaaggtggatcgcgtccgtattggtttgtgctgacatccgagagtatctcctggtataaggacgaggatgagaaggagaagaaatttatgctgccgttggatggtttaaaattgcgtgatattgaacagggatttatgtcaatgtctagacgtgttacatttgctttatttagtcccgatggacgtaatgtttataaggactacaaacaactggagctctcgtgcgagactgtggaggatgtggagtcctggaaagcatccttcctgcgagctggtgtctatcccgaaaagcaggagactcaggaaaacggcgatgagtcggccagcgaggagagttccagcgatccacagctggagcggcaggtcgagaccattcgtaatctggttgattcctacatgaagatcgttacgaagaccacccgtgacatggtgcccaaagcaattatgatgctgatcatcaacaatgccaaggactttatcaatggcgagctgctggcacatctttacgcgtccggcgatcaggcccagatgatggaggagtcggctgaatcggcaacacggcgagaggaaatgctgcgcatgtaccgtgcttgcaaggatgcattacagataataggtgacgtatcaatggccaccgtgtcgtcgccattgccaccgccggttaagaacgattggctgcccagcggcttggacaacccacgtctttctcctccaagtccgggtggcgttcgaggaaagccaggacctcccgcacagagctccttgggtggacggaacccaccattgccaccatctacaggtcgcccagcgccagcgattccgaatcgcccaggaggtggggcaccaccactacccggcggtcgtcccggaggatctcttcccccaccaatgctaccatcccgtgtgtccggagctgtcggcggcgccattgtccagcaatcgggagccaatcgctatgtgccggagagtatgcgtggacaggtgaaccaggcggtgggccaggcagccatcaatgagttgtccaatgccttcagttcgcgattcaagtaatggccgcgtaattgagtgcacagcatgccagagtatgcttaattattaacagaaacagaaacttagagagccagcacgcgaaacccctccccccaccaaccatatcacatcatttttttttttatcaaagatgtaaatcccaaaaagatggcaattcccatccagttgtttcgaaaattcgatggaaaaatttaaaaagtgttttacacatgatcttttggacaattcaacattcaaattagtgctttcgaattaagtttatcggggataccatttactttttaaagtatatttgaacgtgaaataaaaaaaaaaaaatttattttcc

SEQ ID NO: 160 - protein, 877 aa, D. melanogaster
MDSLITIVNKLQDAFTSLGVHMQLDLPQIAVVGGQSAGKSSVLENFVGKDFLPRGSGIVTRRPLILQLINGVTEYGEFLHIKGKKFSSFDEIRKEIEDETDRVTGSNKGISNIPINLRVYSPHVLNLTLIDLPGLTKVAIGDQPVDIEQQIKQMIFQFIRKETCLILAVTPANTDLANSDALKLAKEVDPQGVRTIGVITKLDLMDEGTDARDILENKLLPLRRGYIGVVNRSQKDIEGRKDIHQALAAERKFFLSHPSYRHMADRLGTPYLQRVLNQQLTNHIRDTLPGLRDKLQKQMLTLEKEVEEFKHFQPGDASIKTKAMLQMIQQLQSDFERTIEGSGSALVNTNELSGGAKINRIFHERLRFEIVKMACDEKELRREISFAIRNIHGIRVGLFTPDMAFEAIVKRQIALLKEPVIKCVDLVVQELSVVVRMCTAKMSRYPRLREETERIITTHVRQREHSCKEQILLLIDFELAYMNTNHEDFIGFANAQNKSENANKTGTRQLGNQVIRKGHMVIQNLGIMKGGSRPYWFVLTSESISWYKDEDEKEKKFMLPLDGLKLRDIEQGFMSMSRRVTFALFSPDGRNVYKDYKQLELSCETVEDVESWKASFLRAGVYPEKQETQENGDESASEESSSDPQLERQVETIRNLVDSYMKIVTKTTRDMVPKAIMMLIINNAKDFINGELLAHLYASGDQAQMMEESAESATRREEMLRMYRACKDALQIIGDVSMATVSSPLPPPVKNDWLPSGLDNPRLSPPSPGGVRGKPGPPAQSSLGGRNPPLPPSTGRPAPAIPNRPGGGAPPLPGGRPGGSLPPPMLPSRVSGAVGGAIVQQSGANRYVPESMRGQVNQAVGQAAINELSNAFSSRFK

SEQ ID NO: 161 - DNA, 3350 bp, C. elegans
cagccatgtcgtggcaaaaccagggaatgcaggcgttgatccctgtgatcaatcgtgtccaggacgccttctcccagctgggcacatctgtcagcttcgaacttccacagatcgccgtcgtcggaggacagtccgctggaaagtcgtcggtgctggagaattttgtcggaaaagacttcttgccacgtggatcaggaatcgtaacacgtcgtccacttattttgcagcttattcaagatcgcaatgagtacgccgagttcctacacaagaagggtcatcgctttgtggattttgatgcagttcggaaagagattgaggatgagactgatcgtgtcactggacagaataagggaatcagtccacatccaatcaacttgcgtgtcttttctccaaatgttctaaatctgacactcatcgatttgcccggtctcacaaaagtgcccgtcggagatcaaccagcagatattgagcaacagatccgtgacatgattctcacattcatcaaccgtgagacttgcctcattcttgccgtcactccggccaacagcgatctcgccacttcggatgcgttgaaacttgcgaaggaagtcgatccacagggtcttcgcacgattggagtcctcaccaaacttgacttgatggacgagggaaccgatgctcgcgagatcctcgagaacaagctgttcacacttcgtcgtggctacgtcggagttgtcaatcgtgggcagaaggatattgtcggtcgcaaggatattagagctgctttggacgccgagagaaagttcttcatctcacacccatcctaccgacatatggctgatcggttgggaacaagctaccttcaacacactcttaatcaacagctcaccaatcatatccgtgatacactgccaacacttcgtgatagtcttcaaaagaagatgtttgctatggaaaaggatgtggccgagtacaagaactaccagccaaatgatccaggccgcaagaccaaggctcttttgcaaatggttacccagttcaatgctgacattgagcgctccattgaaggttcctctgcaaagctggtttcaaccaatgagctcagtggaggagcccgtatcaatcggcttttccatgagcgtttcccatttgagattgttaaaatggaaattgacgagaaagaaatgcgcaaagaaatccagtatgccatcagaaacattcacggtatccgcgtcggtctcttcactccggatatggcgtttgaggcaattgccaaaaagcaaatcacccgtctgaaggagccatcgttgaaatgcgttgatctggtggtcaacgagttggctaatgtgatcagacagtgcgctgacactatggctagatatccacgtcttcgtgacgagctggaaagaatcgtcgtctcgcatatgcgtgaacgtgagcaaattgccaagcagcaaattgggctcattgttgactacgaactcgcttatatgaacacaaaccatgaggatttcattggattcagcaatgctgaagcaaaagcctcccaaggacaatcagcgaagaagaatcttggaaaccaggtgatcagaaagggctggctctcactgagcaacgtatcgtttgtgcgtggctccaaggacaattggtttgtgctcatgtcggacagtttgagttggtacaaagatgatgaggagaaggagaagaagtacatgctcccattggatggtgtcaagctgaaggatattgagggtggatttatgtctcgtaaccacaagtttgctctgttctaccccgacggaaagaacatctacaaggattacaagcagcttgagttgggatgcaccaatttggacgaaattgatgcgtggaaggcttcattcttgcgtgctggtgtctatccagaaaagcagaaggcacaggaagatgagtcccaacaagagatggaggatacctcgattgatccacaacttgagagacaggtggagacaatccgtaatttggttgattcctacatgagaatcattaccaagacaattaaggacctggttccaaaggcggtgatgcatttgattgttaaccagacaggtgagttcatgaaagatgaacttttggcccatctctaccaatgcggcgacactgatgctctcatggaggaatctcaaatagaagcccagaagcgcgaggagatgctccgaatgtaccatgcttgcaaggaggcgctcccgattatctctgaagtcaacatgagcacccttggcgaccagccgccgccattgccgatgtctgactaccgcccacacccatctggaccttcaccggtgccgcgtccggctcctgctccaccaggcggacgtcaggccccaatgccaccacgcggaggacccggtgccccaccaccaccaggcatgagaccaccaccaggtgcgccaggaggcggcggtggcatgtacccaccgttgattccaacgcgtccgggaccaggcggcccaccacccaacatggcaccgcctagataataatgttgtgaataattcccccactgaccaaagaagagtaccgacaccatccaacggagcaccagagattccagctcgcccgcaagtccccaagagacctttctaaatgcttttacatctccatcctagctcacttgtcatactattaccgttcccatagtaattgtacaatgcccccgttttatcaagcatttcttttttttttttgagaatttttaaattaatcaatatcccgagttcttccaccttttgagcaagtctcgttgaggattgcattggtcccagcgctaactaggcgtcaattcatccacctctccattctctctcccgttccaactgtgtttttctttgtcagcggcgaattttccacaaccaatcgagtcctatttctctttttttcccccacctcccaataattttatttgcatgttttataaactcactttgttcaataatttattgattttattttttctatgtatttgaaactttacatttttcctactcttctatagcaatactttgtcacttacaataacccccccccccccagtctgcctatatttatttagatgttctttatggccacccaatgtataaactcttttagtgcatttgtaggttctttcttcatcccacccaattccccctccaccacaaatcccagttttgattattttaagcccatatttataagaagtaaatatatttatagatttttcttagattataagctcttctaggttttccctcgttgagaagaatgatctgaaaaagcctaaattatagatttttgaaacgtattgtgtagaatttgtatttattcgtgaaagtggaataaagttaaaacgg

SEQ ID NO: 162 - protein, 830 aa, C. elegans
MSWQNQGMQALIPVINRVQDAFSQLGTSVSFELPQIAVVGGQSAGKSSVLENFVGKDFLPRGSGIVTRRPLILQLIQDRNEYAEFLHKKGHRFVDFDAVRKEIEDETDRVTGQNKGISPHPINLRVFSPNVLNLTLIDLPGLTKVPVGDQPADIEQQIRDMILTFINRETCLILAVTPANSDLATSDALKLAKEVDPQGLRTIGVLTKLDLMDEGTDAREILENKLFTLRRGYVGVVNRGQKDIVGRKDIRAALDAERKFFISHPSYRHMADRLGTSYLQHTLNQQLTNHIRDTLPTLRDSLQKKMFAMEKDVAEYKNYQPNDPGRKTKALLQMVTQFNADIERSIEGSSAKLVSTNELSGGARINRLFHERFPFEIVKMEIDEKEMRKEIQYAIRNIHGIRVGLFTPDMAFEAIAKKQITRLKEPSLKCVDLVVNELANVIRQCADTMARYPRLRDELERIVVSHMREREQIAKQQIGLIVDYELAYMNTNHEDFIGFSNAEAKASQGQSAKKNLGNQVIRKGWLSLSNVSFVRGSKDNWFVLMSDSLSWYKDDEEKEKKYMLPLDGVKLKDIEGGFMSRNHKFALFYPDGKNIYKDYKQLELGCTNLDEIDAWKASFLRAGVYPEKQKAQEDESQQEMEDTSIDPQLERQVETIRNLVDSYMRIITKTIKDLVPKAVMHLIVNQTGEFMKDELLAHLYQCGDTDALMEESQIEAQKREEMLRMYHACKEALPIISEVNMSTLGDQPPPLPMSDYRPHPSGPSPVPRPAPAPPGGRQAPMPPRGGPGAPPPPGMRPPPGAPGGGGGMYPPLIPTRPGPGGPPPNMAPPR

SEQ ID NO: 163 - protein, 7 aa, Artificial Sequence
  Virus-derived peptide
RPDPTAP

SEQ ID NO: 164 - protein, 7 aa, Artificial Sequence
  Virus-derived peptide
RPLPVAP

SEQ ID NO: 165 - protein, 7 aa, Artificial Sequence
  Virus-derived peptide
RPEPTAP

SEQ ID NO: 166 - protein, 4 aa, Artificial Sequence
  Virus-derived peptide
YEDL

SEQ ID NO: 167 - protein, 7 aa, Artificial Sequence
  Virus-derived peptide
PTAPPEY

SEQ ID NO: 168 - protein, 10 aa, Artificial Sequence
  Virus-derived peptide
RPEPTAPPEE

SEQ ID NO: 169 - protein, 4 aa, Artificial Sequence
  Virus-derived peptide
  VARIANT 2: Xaa = Thr or Ser
PXAP

SEQ ID NO: 170 - protein, 5 aa, Artificial Sequence
  Virus-derived peptide
PFRDY

SEQ ID NO: 171 - protein, 7 aa, Artificial Sequence
  Virus-derived peptide
RQGPKEP

SEQ ID NO: 172 - protein, 9 aa, Artificial Sequence
  Virus-derived peptide
RQGPKEPFR

SEQ ID NO: 173 - protein, 7 aa, Artificial Sequence
  Virus-derived peptide
  VARIANT 2, 3: Xaa = Any Amino Acid
  VARIANT 5: Xaa= Thr or Ser
RXXPXAP

SEQ ID NO: 174 - protein, 5 aa, Artificial Sequence
  Virus-derived peptide
PFRDY

SEQ ID NO: 175 - protein, 4 aa, Artificial Sequence
  Virus-derived peptide
PTAP